(19) United States
(12) Patent Application Publication
(10) Pub. No.: US 2026/0211321 A1
(43) Pub. Date: Jul. 23, 2026

(54) RESIST COMPOSITION AND METHOD FOR PRODUCING RESIST PATTERN, AND SALT AND RESIN

(71) Applicant: SUMITOMO CHEMICAL COMPANY, LIMITED, Tokyo (JP)

(72) Inventors: Shohei TERAHIGASHI, Osaka (JP); Wataru NOJO, Osaka (JP); Koji ICHIKAWA, Osaka (JP)

(73) Assignee: SUMITOMO CHEMICAL COMPANY, LIMITED, Tokyo (JP)

(21) Appl. No.: 19/448,912

(22) Filed: Jan. 14, 2026

(30) Foreign Application Priority Data
Jan. 17, 2025 (JP) 2025-007155
Jul. 10, 2025 (JP) 2025-116777

(51) Int. Cl. G03F 7/004; C07C 309/12; C07C 309/15; C07C 309/23; C07C 309/58; C07C 381/12; C07D 327/08; C08F 220/18; G03F 7/038; G03F 7/039

(52) U.S. Cl. CPC G03F 7/0045; C07C 309/12; C07C 309/15; C07C 309/23; C07C 309/58; C07C 381/12; C07D 327/08; C08F 220/1808; G03F 7/0382; G03F 7/0388; G03F 7/0397; C07C 2603/74; C08F 2800/10

(57) ABSTRACT

A resist composition includes a salt represented by formula (I) or a resin that includes a structural unit represented by formula (IP):

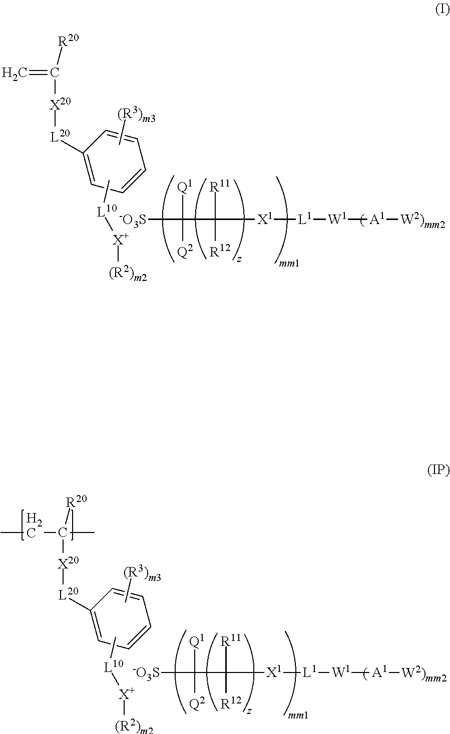

wherein Q¹, Q², R¹¹ and R¹² each represent a H atom or the like; X¹ represents a single bond or the like; W¹ represents a single bond or a substituted/unsubstituted cyclic hydrocarbon group; L¹, A¹, L¹⁰ and L²⁰ each represent a single bond or a substituted/unsubstituted hydrocarbon group; W² represents a cyclic hydrocarbon group having an I atom; R² represents a substituted/unsubstituted hydrocarbon group; z represents an integer of 0 to 6, mm2 represents an integer of 1 to 5, m3 represents an integer of 0 to 4, mm1 represents 0 or 1, m2 represents 1 or 2; R³ represents a halogen atom or a substituted/unsubstituted hydrocarbon group; R²⁰ represents a H atom or the like; and X²⁰ represents a single bond, —O— or the like.

### TECHNICAL FIELD

The present invention relates to a resist composition and a method for producing a resist pattern, and a salt and a resin.

### BACKGROUND ART

Patent Literature 1 discloses a resist composition containing a resin that includes a structural unit derived from a salt represented by the following formula.

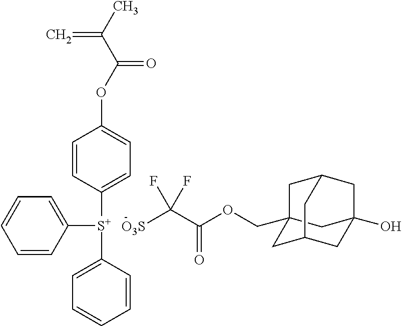

Patent Literature 2 discloses a resist composition containing a resin that includes a structural unit derived from a salt represented by the following formula.

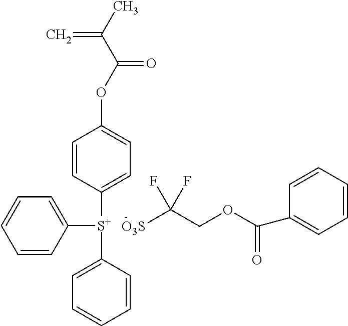

### CITATION LIST

### Patent Literature

Patent Literature 1: Japanese Patent Laid-Open No. 2011-037836
Patent Literature 2: Japanese Patent Laid-Open No. 2010-132852

### SUMMARY OF INVENTION

### Problems to be Solved by Invention

An object of the present invention is to provide a resist composition that forms a resist pattern with better CD uniformity (CDU) than resist patterns formed from the above resist compositions.

### Means to Solve the Problems

The present invention includes the following inventions.
[1] A resist composition containing a salt represented by formula (I) or a resin that includes a structural unit represented by formula (IP):

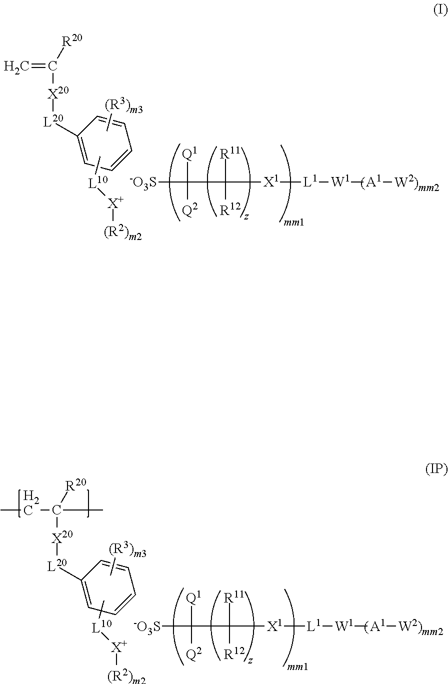

wherein
Q¹, Q², R¹¹, and R¹² each independently represent a hydrogen atom, a fluorine atom, an alkyl group having 1 to 6 carbon atoms, or a perfluoroalkyl group having 1 to 6 carbon atoms;
z represents an integer of 0 to 6, and when z is 2 or larger, a plurality of R¹¹ and R¹² may be the same or different from each other;
X¹ represents a single bond, —O—, —CO—, —S—, —SO—, —SO₂—, —NR⁷—, or a combined group of these groups;
mm1 represents 0 or 1;
L¹ represents a single bond, or a hydrocarbon group having 1 to 40 carbon atoms and optionally having a substituent, and —CH₂— contained in the hydrocarbon group is optionally replaced with —O—, —S—, —SO₂—, —SO—, —NR⁷— or —C)—;
R⁷ represents a hydrogen atom or an alkyl group having 1 to 6 carbon atoms;
W¹ represents a single bond, or a cyclic hydrocarbon group having 3 to 36 carbon atoms and optionally having a substituent, and —CH₂— contained in the cyclic hydrocarbon group is optionally replaced with —O—, —S—, —SO₂—, —SO—, —NR⁷— or —CO—;
A¹ represents a single bond, or a hydrocarbon group having 1 to 20 carbon atoms, the hydrocarbon group optionally has a substituent, and —CH₂-contained in the hydrocarbon group is optionally replaced with —O—, —S—, —SO₂—, —SO—, —NR⁷— or —CO—;
W² represents a cyclic hydrocarbon group having 3 to 36 carbon atoms and an iodine atom, —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom;
mm2 represents an integer of 1 to 5, and when mm2 is 2 or larger, a plurality of groups in parentheses may be the same or different from each other;
R² represents a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
m2 represents 1 or 2, and when m2 is 1, X is an iodine atom, and when m2 is 2, X is a sulfur atom and two R² optionally together form a ring containing a sulfur atom, where —CH₂— contained in the ring is optionally replaced with —O—, —S—, —SO₂—, —SO—, —NR⁷— or —CO—;
R³ represents a halogen atom, or a hydrocarbon group having 1 to 18 carbon atoms, the hydrocarbon group optionally has a substituent, and —CH₂-contained in the hydrocarbon group is optionally replaced with —O—, —S—, —SO₂—, —SO—, —NR⁷— or —CO—;
m3 represents an integer of 0 to 4, and when m3 is 2 or larger, a plurality of R³ may be the same or different from each other;
L¹⁰ and L²⁰ each independently represent a single bond, or a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
R²⁰ represents a hydrogen atom, a halogen atom, or an alkyl group having 1 to 6 carbon atoms and optionally having a halogen atom; and
X²⁰ represents a single bond, —O—, —CO—, —NR⁷—, —S—, —SO—, —SO₂—, a phenylene group optionally having a substituent, or a combined group of these groups.
[2] The resist composition according to [1], wherein X²⁰ is a single bond or a group represented by any one of formulas (X²⁰-1) to (X²⁰-10):

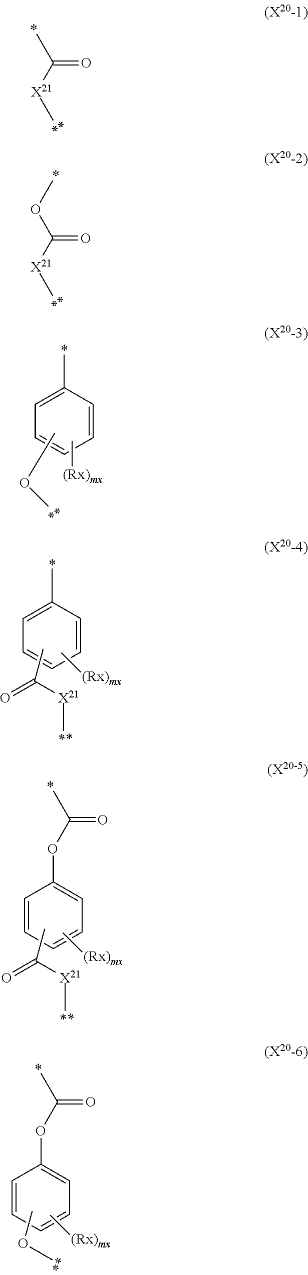

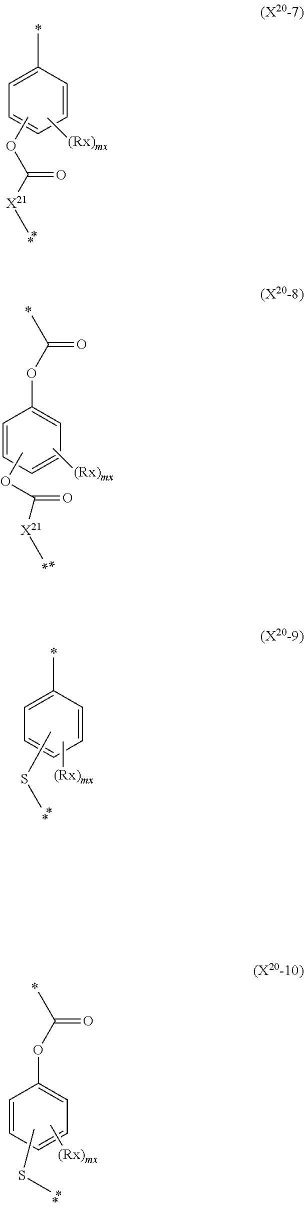

wherein
* and ** are bonding sites, and * represents a bonding site to a carbon atom to which R²⁰ is bonded;
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms;
mx represents an integer of 0 to 4; when mx is 2 or larger, a plurality of Rx may be the same or different from each other;
X²¹ represents —O— or —NR⁷—; and
R⁷ represents a hydrogen atom or an alkyl group having 1 to 6 carbon atoms.
[3] The resist composition according to [1] or [2], wherein L¹ is a single bond or a chain hydrocarbon group having 1 to 10 carbon atoms and optionally having a substituent, where —CH₂— contained in the chain hydrocarbon group is optionally replaced with —O—, —S—, —SO₂—, —SO—, —NR⁷— or —CO—, and W¹ is a cyclic hydrocarbon group having 3 to 18 carbon atoms and optionally having a substituent, where —CH₂— contained in the cyclic hydrocarbon group is optionally replaced with —O—, —S—, —SO₂—, —SO—, —NR⁷— or —CO—.
[4] The resist composition according to any one of [1] to [3], wherein W² is a cyclic hydrocarbon group having 5 to 18 carbon atoms and an iodine atom, where the cyclic hydrocarbon group optionally has a substituent other than the iodine atom.
[5] The resist composition according to any one of [1] to [4], wherein the resist composition further contains a resin that includes a structural unit (a1) having an acid labile group, or the resin that includes a structural unit represented by formula (IP) further includes a structural unit (a1) having an acid labile group.
[6] The resist composition according to [5], wherein the structural unit (a1) having an acid labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5), and a structural unit represented by formula (a1-6):

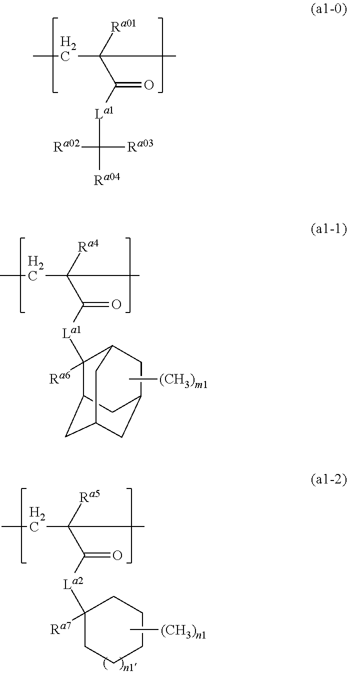

wherein
L^{a01}, L^{a1} and L^{a2} each independently represent —O— or *—O—(CH₂)ₖ₁—CO—O—, k1 represents an integer of 1 to 7, and * represents a bonding site to —CO—;
R^{a01}, R^{a4} and R^{a5} each independently represent a hydrogen atom, a halogen atom, or an alkyl group having 1 to 6 carbon atoms and optionally having a halogen atom;
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or a combined group of these groups, and the alkyl group, the alicyclic hydrocarbon group and the aromatic hydrocarbon group optionally have a halogen atom;
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or a group formed by combining these groups, and the alkyl group, the alkenyl group, the alicyclic hydrocarbon group, and the aromatic hydrocarbon group optionally have a halogen atom;
m1 represents an integer of 0 to 14;
n1 represents an integer of 0 to 14; and
n1′ represents an integer of 0 to 3,

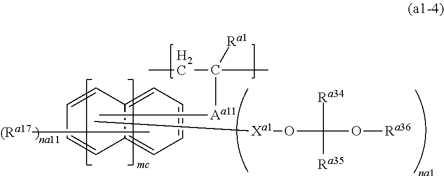

wherein
R^{a1} represents a hydrogen atom, a halogen atom, or an alkyl group having 1 to 6 carbon atoms and optionally having a halogen atom;
R^{a17} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group;
A^{a11} represents a single bond or an alkanediyl group having 1 to 12 carbon atoms, and —CH₂— contained in the alkanediyl group is optionally replaced with —O—, —CO— or —NR^{a18}—;
R^{a18} represents a hydrogen atom or an alkyl group having 1 to 6 carbon atoms;
X^{a1} represents a single bond or a carbonyl group;
R^{a34} and R^{a35} each independently represent a hydrogen atom or a hydrocarbon group having 1 to 12 carbon atoms, and R^{a36} represents a hydrocarbon group having 1 to 20 carbon atoms, or R^{a34} represents a hydrogen atom or a hydrocarbon group having 1 to 12 carbon atoms, and R^{a35} and R^{a36} are bonded to each other to form a divalent heterocyclic group having 3 to 20 carbon atoms together with —C—O— to which they are bonded, and —CH₂— contained in the hydrocarbon group and the divalent heterocyclic group is optionally replaced with —O— or —S—;
na1 represents an integer of 1 to 5, and when na1 is 2 or larger, a plurality of groups in parentheses may be the same or different from each other;
na11 represents an integer of 0 to 4, and when na11 is 2 or larger, a plurality of R^{a17} may be the same or different from each other; and
mc represents an integer of 0 to 2,

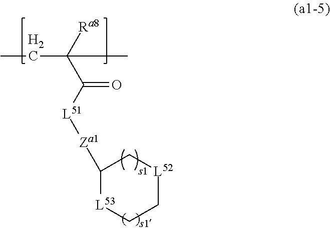

wherein
R^{a8} represents an alkyl group having 1 to 6 carbon atoms and optionally having a halogen atom, a hydrogen atom or a halogen atom;
Z^{a1} represents a single bond or *—(CH₂)ₕ₃—CO-L⁵⁴-, h3 represents an integer of 1 to 4, and * represents a bonding site to L⁵¹;
L⁵¹, L⁵², L⁵³ and L⁵⁴ each independently represent —O— or —S—;
s1 represents an integer of 1 to 3; and
s1′ represents an integer of 0 to 3, and

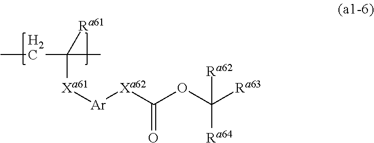

wherein
R^{a61} represents a hydrogen atom, a halogen atom, or an alkyl group having 1 to 6 carbon atoms and optionally having a halogen atom;
R^{a62}, R^{a63} and R^{a64} each independently represent an alkyl group having 1 to 6 carbon atoms or a cyclic hydrocarbon group having 3 to 18 carbon atoms and optionally having a substituent, or R^{a62} and R^{a63} are bonded to each other to form a ring having 3 to 20 carbon atoms together with a carbon atom to which they are bonded, and R^{a64} represents an alkyl group having 1 to 6 carbon atoms or a cyclic hydrocarbon group having 3 to 18 carbon atoms and optionally having a substituent;
X^{a61} represents a single bond, —CO—O—*, or —CO—NR^{a65}—*, * represents a bonding site to Ar, and R^{a65} represents a hydrogen atom or an alkyl group having 1 to 6 carbon atoms;
X^{a62} represents a single bond, *—O-L^{a61}- or *—CO—O-L^{a62}-, * represents a bonding site to Ar, and L^{a61} and L^{a62} each independently represent an alkanediyl group having 1 to 4 carbon atoms; and
Ar represents an aromatic hydrocarbon group having 6 to 20 carbon atoms and optionally having a substituent.
[7] The resist composition according to any one of [1] to [6], wherein the resist composition further contains a resin that includes a structural unit represented by formula (a2-A), or
the resin that includes a structural unit represented by formula (IP) further includes a structural unit represented by formula (a2-A):

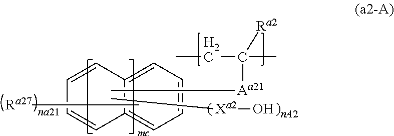

wherein
R^{a2} represents a hydrogen atom, a halogen atom, or an alkyl group having 1 to 6 carbon atoms and optionally having a halogen atom;
R^{a27} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group;
A^{a21} represents a single bond or an alkanediyl group having 1 to 12 carbon atoms, and —CH₂— contained in the alkanediyl group is optionally replaced with —O—, —CO— or —NR^{a28}—;
R^{a28} represents a hydrogen atom or an alkyl group having 1 to 6 carbon atoms;
X^{a2} represents a single bond or a carbonyl group;
nA2 represents an integer of 1 to 5, and when nA2 is 2 or larger, a plurality of groups in parentheses may be the same or different from each other;
na21 represents an integer of 0 to 4, and when na21 is 2 or larger, a plurality of R^{a27} may be the same or different from each other; and
mc represents an integer of 0 to 2.
[8] The resist composition according to any one of [1] to [7], further containing a salt that generates an acid weaker in acidity than an acid generated from the salt represented by formula (I) or the resin that includes a structural unit represented by formula (IP)
[9]A method for producing a resist pattern, including:
(1) a step of applying the resist composition according to any one of [1] to [8] onto a substrate;
(2) a step of drying the applied resist composition to form a composition layer;
(3) a step of exposing the composition layer;
(4) a step of heating the exposed composition layer; and
(5) a step of developing the composition layer after heating.
[10] A salt represented by the above formula (I).
[11] The salt according to [10], wherein X²⁰ is a single bond or a group represented by any one of the above formulas (X²⁰-1) to (X²⁰-10).
[12] The salt according to [10] or [11], wherein L¹ is a single bond or a chain hydrocarbon group having 1 to 10 carbon atoms and optionally having a substituent, where —CH₂— contained in the chain hydrocarbon group is optionally replaced with —O—, —S—, —SO₂—, —SO—, —NR⁷— or —CO—, and W¹ is a cyclic hydrocarbon group having 3 to 18 carbon atoms and optionally having a substituent, where —CH₂— contained in the cyclic hydrocarbon group is optionally replaced with —O—, —S—, —SO₂—, —SO—, —NR⁷— or —CO—.
[13] The salt according to any one of [10] to [12], wherein W² is a cyclic hydrocarbon group having 5 to 18 carbon atoms and an iodine atom, where the cyclic hydrocarbon group optionally has a substituent other than the iodine atom.
[14]A resin including a structural unit represented by the above formula (IP).

### Effects of Invention

By the use of a resist composition containing the salt or structural unit of the present invention, a resist pattern with good CD uniformity (CDU) can be produced.

### EMBODIMENTS FOR CARRYING OUT INVENTION

In the present specification, the “(meth)acrylic monomer” means “at least one of an acrylic monomer and a methacrylic monomer”. The notations of “(meth)acrylate”, “(meth)acrylic acid” and the like also represent the same meaning. In the groups described in the present specification, those that can have both a straight-chain structure and a branched structure may be any one of them. When —CH₂— contained in a hydrocarbon group or the like is replaced with —O—, —S—, —CO—, —SO—, —NR^{X}— (where X is any symbol) or —SO₂—, the same example applies to each group, and the number of carbon atoms before replacement is defined as the number of carbon atoms in the hydrocarbon group or the like. The term “combined group” means a group in which two or more of the exemplified groups are bonded, and the valences of these groups may be appropriately changed depending on the bonding form. The “derived” or “induced” means that a polymerizable C═C bond contained in the molecule becomes a —C—C— group (single bond) by polymerization. When stereoisomers exist, all stereoisomers are included. In each group, hydrogen atoms at any positions and in any number, which are contained in the group, are substituted with bonding hands, in some cases, depending on the number of substituents and the like. The number of carbon atoms in the substituent is not included in the number of carbon atoms in the substituted group.

In the present specification, the “acid labile group” means a group that has a leaving group, which, upon contact with an acid (for example, trifluoromethane sulfonic acid or the like), undergoes detachment of the leaving group to form a hydrophilic group (for example, hydroxy group, carboxy group or the like). The “base labile group” means a group that has a leaving group, which, upon contact with a base (for example, trimethylamine, tetramethylammonium hydroxide or the like), undergoes detachment of the leaving group to form a hydrophilic group (for example, carboxy group, hydroxy group or the like).

The “solid content of the resist composition” means a sum of components excluding the solvent (E) which will be described later, from the total amount of the resist composition.

### [Salt Represented by Formula (I)]

The present invention relates to a salt represented by formula (I) (hereinafter referred to as “salt (I)” in some cases).

In the salt (I), a side having a negative charge is referred to as “anion (I)”, and a side having a positive charge is referred to as “cation (I)”, in some cases.

In formula (I), examples of the perfluoroalkyl group of Q¹, Q², R¹¹ and R¹² include a trifluoromethyl group, a perfluoroethyl group, a perfluoropropyl group, a perfluoroisopropyl group, a perfluorobutyl group, a perfluoro-sec-butyl group, a perfluoro-tert-butyl group, a perfluoropentyl group and a perfluorohexyl group. The number of carbon atoms of the perfluoroalkyl group is preferably 1 to 4, and is more preferably 1 to 3.

Examples of the alkyl group of Q¹, Q², R¹¹ and R¹² include a methyl group, an ethyl group, a propyl group, an isopropyl group, a butyl group, a sec-butyl group, a tert-butyl group, a pentyl group and a hexyl group. The number of carbon atoms of the alkyl group is preferably 1 to 4, and is more preferably 1 to 3.

At least one of Q¹ and Q² preferably contains a fluorine atom or a perfluoroalkyl group; at least one of Q¹ and Q² is more preferably a fluorine atom or a perfluoroalkyl group; Q¹ and Q² are each independently further preferably a trifluoromethyl group or a fluorine atom; and both of Q¹ and Q² are further more preferably fluorine atoms.

R¹¹ and R¹² are each independently preferably a hydrogen atom, a fluorine atom, a methyl group or a trifluoromethyl group, are each more preferably a hydrogen atom or a trifluoromethyl group, and are each further preferably a hydrogen atom.

z is preferably an integer of 0 to 4, is more preferably an integer of 0 to 3, is further preferably an integer of 0 to 2, and is further more preferably 0 or 1.

X¹ is preferably a single bond, —O—, —CO—, —NR⁷—, or a combined group of these groups, is more preferably a single bond, *—CO—O—, *—O—CO—, *—O—, * O—CO—O—, —NR⁷—, *—CO—NR⁷—, *—NR⁷—CO—, *—NR⁷—CO—O— or *—O—CO—NR⁷—, is further preferably *—CO—O—, *—O—CO—, *—O— or *—O—CO—O—, is still more preferably *—CO—O—, *—O—CO—O— or *—O—CO—, and is still further preferably * CO—O— or *—O—CO—, where * represents a bonding site to C(R¹¹)(R¹²) or C(Q¹)(Q²).

Examples of the hydrocarbon group in L¹ include a straight-chain or branched chain hydrocarbon group such as an alkanediyl group, a monocyclic or polycyclic (including a spiro ring, a condensed ring or a bridged ring), saturated or unsaturated alicyclic hydrocarbon group, and an aromatic hydrocarbon group, and it may be a combined group of two or more of these groups (for example, a hydrocarbon group formed from an alicyclic hydrocarbon group or an aromatic hydrocarbon group and a chain hydrocarbon group). The chain hydrocarbon group and the alicyclic hydrocarbon group are also referred to as an aliphatic hydrocarbon group, and the alicyclic hydrocarbon group and the aromatic hydrocarbon group are also referred to as a cyclic hydrocarbon group. The number of carbon atoms of the hydrocarbon group may be 1 to 36, is preferably 1 to 24, is more preferably 1 to 20, is further preferably 1 to 18, and is further more preferably 1 to 12.

Examples of the alkanediyl group include: a straight-chain alkanediyl group such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group; and

a branched alkanediyl group such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The terminal of the branched alkanediyl group may be an alkyl group such as a methyl group.

The number of carbon atoms of the chain hydrocarbon group may be 1 to 24, is preferably 1 to 18, is more preferably 1 to 12, is further preferably 1 to 10, is further more preferably 1 to 8, is still more preferably 1 to 6, and is still further preferably 1 to 4.

Examples of the monocyclic or polycyclic divalent alicyclic hydrocarbon group include the following groups. The bonding site can be set to any position.

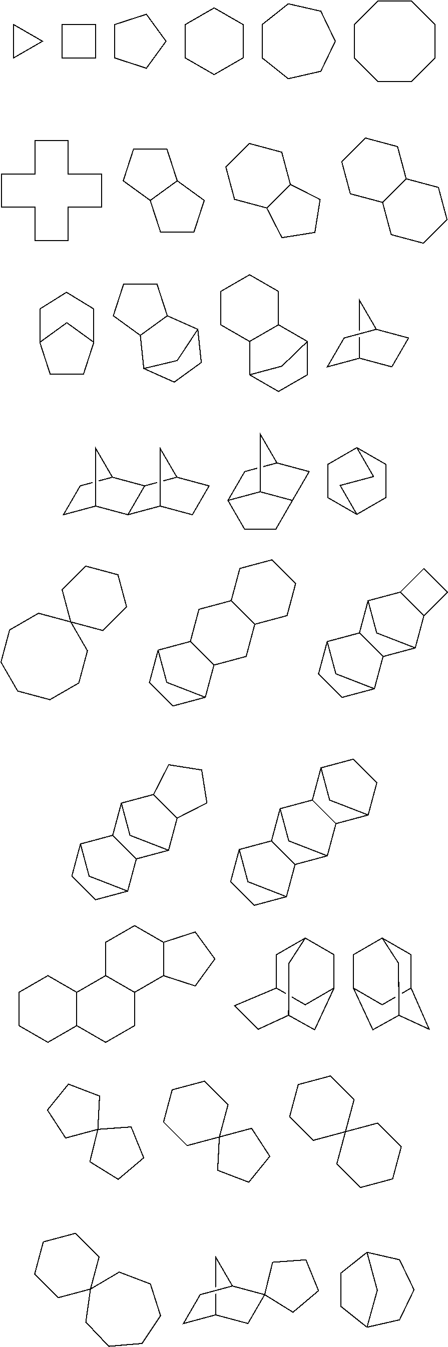

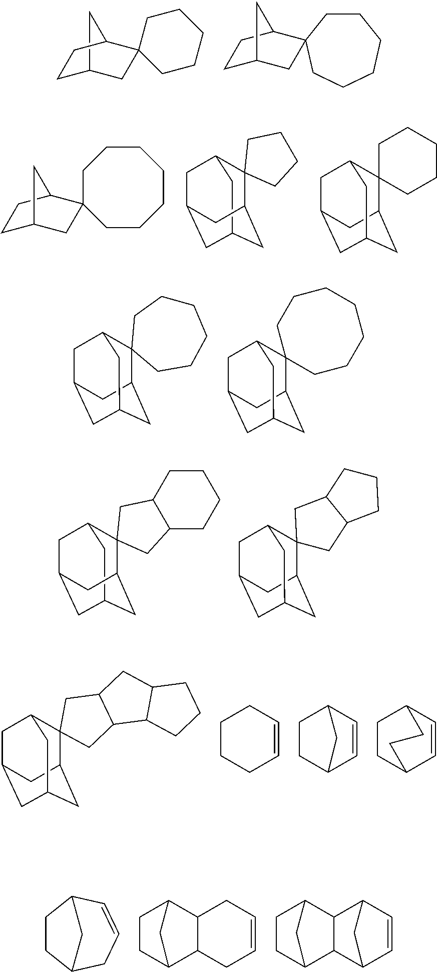

Specific examples of the alicyclic hydrocarbon group include: a monocyclic divalent alicyclic hydrocarbon group that is a monocyclic cycloalkanediyl group such as a cyclobutane-1,3-diyl group, a cyclopentane-1,3-diyl group, a cyclohexane-1,4-diyl group and a cyclooctane-1,5-diyl group; and

a polycyclic divalent alicyclic hydrocarbon group that is a polycyclic cycloalkanediyl group such as a spiro ring having a cycloalkyl group bonded by spiro to each of a cycloalkyl group, a norbornyl group or an adamantyl group, such as a norbornane-1,4-diyl group, a norbornane-2,5-diyl group, an adamantane-1,5-diyl group, an adamantane-2,6-diyl group, a spirocyclohexane-1,2′-cyclopentane group, or a spiroadamantane-2,3′-cyclopentane group. The number of carbon atoms of the alicyclic hydrocarbon group may be 3 to 24, is preferably 3 to 18, is more preferably 3 to 16, and is further preferably 3 to 12.

Examples of the aromatic hydrocarbon group include an aromatic hydrocarbon group such as an arylene group including a phenylene group, a naphthylene group, an anthrylene group, a biphenylene group and a phenanthrylene group. The number of carbon atoms of the aromatic hydrocarbon group may be 6 to 24, is preferably 6 to 18, is more preferably 6 to 14, and is further preferably 6 to 10.

Examples of the combined group of two or more include a combined group of an alicyclic hydrocarbon group and a chain hydrocarbon group, a combined group of an aromatic hydrocarbon group and a chain hydrocarbon group, a combined group of an alicyclic hydrocarbon group and an aromatic hydrocarbon group, and a combined group of an alicyclic hydrocarbon group, an aromatic hydrocarbon group and a chain hydrocarbon group. In the combination, two or more types of alicyclic hydrocarbon groups, aromatic hydrocarbon groups, and chain hydrocarbon groups may each be combined. In addition, any of the groups may be bonded to X¹ or W¹.

Examples of the combined group of an alicyclic hydrocarbon group and a chain hydrocarbon group include -(divalent alicyclic hydrocarbon group)-(alkanediyl group)-, -(alkanediyl group)-(divalent alicyclic hydrocarbon group)-(alkanediyl group)-, and -(alkanediyl group)-(divalent alicyclic hydrocarbon group)-.

Examples of the combined group of an aromatic hydrocarbon group and a chain hydrocarbon group include -(divalent aromatic hydrocarbon group)-(alkanediyl group)-, -(alkanediyl group)-(divalent aromatic hydrocarbon group)-(alkanediyl group)-, and -(alkanediyl group)-(divalent aromatic hydrocarbon group)-.

Examples of the combined group of an alicyclic hydrocarbon group and an aromatic hydrocarbon group include -(aromatic hydrocarbon group)-(alicyclic hydrocarbon group)-, -(alicyclic hydrocarbon group)-(aromatic hydrocarbon group)-, and -(alicyclic hydrocarbon group)-(aromatic hydrocarbon group)-(alicyclic hydrocarbon group)-.

—CH₂— contained in the hydrocarbon group of L¹ is optionally replaced with —O—, —S—, —SO₂—, —SO—, —NR⁷— or —CO—.

When —CH₂— contained in the hydrocarbon group of L¹ is replaced with —O—, —S—, —SO₂—, —SO—, —NR⁷— or —CO—, the number of carbon atoms before replacement corresponds to the defined number of carbon atoms of the hydrocarbon group.

Examples of the alkyl group of R¹ include a methyl group, an ethyl group, a propyl group, an isopropyl group, a butyl group, a sec-butyl group, a tert-butyl group, a pentyl group and a hexyl group. The number of carbon atoms of the alkyl group is preferably 1 to 4, and is more preferably 1 to 3.

R⁷ is preferably a hydrogen atom or an alkyl group having 1 to 4 carbon atoms, is more preferably a hydrogen atom or an alkyl group having 1 to 3 carbon atoms, is further preferably a hydrogen atom, a methyl group or an ethyl group, and is further more preferably a hydrogen atom or a methyl group.

Examples of the group formed by replacing —CH₂— contained in the hydrocarbon group with —O—, —S—, —SO₂—, —SO—, —NR⁷— or —CO— include: a hydroxy group (a group formed by replacing —CH₂— contained in a methyl group with —O—), a carboxy group (a group formed by replacing —CH₂—CH₂— contained in an ethyl group with —O—CO—), a thiol group (a group formed by replacing —CH₂-contained in a methyl group with —S—), an amino group (a group formed by replacing —CH₂— contained in a methyl group with —NR⁷—), an oxy group (a group formed by replacing —CH₂— contained in a methylene group with —O—), a carbonyl group (a group formed by replacing —CH₂— contained in a methylene group with —CO—), a thio group (a group formed by replacing —CH₂— contained in a methylene group with —S—), a sulfonyl group (a group formed by replacing —CH₂-contained in a methylene group with —SO₂—), a peptide group (a group formed by replacing —CH₂—CH₂— contained in an ethylene group with —CO—NR⁷—), an alkoxy group (a group formed by replacing —CH₂— at any position in an alkyl group with —O—), an alkylthio group (a group formed by replacing —CH₂— at any position contained in an alkyl group with —S—), an alkylsulfonyl group (a group formed by replacing —CH₂— at any position in an alkyl group with —SO₂—), an alkylamino group (a group formed by replacing —CH₂— at any position contained in an alkyl group with —NR⁷—), an alkyl peptide group (a group formed by replacing —CH₂—CH₂— at any position in an alkyl group with —CO—NR⁷—), an alkoxycarbonyl group (a group formed by replacing —CH₂—CH₂— at any position contained in an alkyl group with —O—CO—), an alkylcarbonyl group (a group formed by replacing —CH₂— at any position in an alkyl group with —CO—), an alkylcarbonyloxy group (a group formed by replacing —CH₂—CH₂— at any position contained in an alkyl group with —CO—O—), an alkoxycarbonyloxy group (a group formed by replacing —CH₂—CH₂—CH₂— at any position in an alkyl group with —O—CO—O—), an alkoxyalkoxy group (a group formed by replacing two —CH₂— at any positions contained in an alkyl group with —O—), an alkoxyalkyl group (a group formed by replacing —CH₂— at any position contained in an alkyl group with —O—), a hydroxyalkyl group (a group formed by replacing —CH₂— contained in a methyl group at any position contained in an alkyl group with —O—), an alkanediyloxy group (a group formed by replacing —CH₂— at any position contained in an alkanediyl group with —O—), an alkanediyloxycarbonyl group (a group formed by replacing —CH₂—CH₂— at any position contained in an alkanediyl group with —O—CO—), an alkanediylcarbonyl group (a group formed by replacing —CH₂— at any position contained in an alkanediyl group with —CO—), an alkanediylcarbonyloxy group (a group formed by replacing —CH₂—CH₂— at any position contained in an alkanediyl group with —CO—O—), an alkanediyloxycarbonyloxy group (a group formed by replacing —CH₂—CH₂—CH₂— at any position contained in an alkanediyl group with —O—CO—O—), an alkanediylsulfonyl group (a group formed by replacing —CH₂— at any position contained in an alkanediyl group with —SO₂—), an alkanediylthio group (a group formed by replacing —CH₂— at any position contained in an alkanediyl group with —S—), an alkanediylamino group (a group formed by replacing —CH₂— at any position contained in an alkanediyl group with —NR⁷—), an alkanediyl peptide group (a group formed by replacing —CH₂—CH₂— at any position contained in an alkanediyl group with —CO—NR⁷—), a cycloalkoxy group, a cycloalkylalkoxy group, an aromatic hydrocarbon group-carbonyloxy group, an aromatic hydrocarbon group-carbonyl group, an aromatic hydrocarbon group-oxy group, and a combined group of two or more of these groups.

Examples of the alkoxy group include an alkoxy group having 1 to 39 carbon atoms, and examples thereof include a methoxy group, an ethoxy group, a propoxy group, a butoxy group, a pentyloxy group, a hexyloxy group, an octyloxy group, a 2-ethylhexyloxy group, a nonyloxy group, a decyloxy group and an undecyloxy group. The number of carbon atoms of the alkoxy group may be 1 to 27, is preferably 1 to 11, is more preferably 1 to 6, is further preferably 1 to 4, and is further more preferably 1 to 3.

Examples of the alkylthio group include an alkylthio group having 1 to 39 carbon atoms, and examples thereof include a methylthio group, an ethylthio group, a propylthio group, a butylthio group, a pentylthio group, a hexylthio group, an octylthio group, a 2-ethylhexylthio group, a nonylthio group, a decylthio group and an undecylthio group. The number of carbon atoms of the alkylthio group may be 1 to 27, is preferably 1 to 11, is more preferably 1 to 6, is further preferably 1 to 4, and is further more preferably 1 to 3.

Examples of the alkylsulfonyl group include an alkylsulfonyl group having 1 to 39 carbon atoms, and examples thereof include a methylsulfonyl group, an ethylsulfonyl group, a propylsulfonyl group, a butylsulfonyl group, a pentylsulfonyl group, a hexylsulfonyl group, an octylsulfonyl group, a 2-ethylhexylsulfonyl group, a nonylsulfonyl group, a decylsulfonyl group and an undecylsulfonyl group. The number of carbon atoms of the alkylsulfonyl group may be 1 to 27, is preferably 1 to 11, is more preferably 1 to 6, is further preferably 1 to 4, and is further more preferably 1 to 3.

Examples of the alkylamino group include an alkylamino group having 1 to 39 carbon atoms, and examples thereof include a methylamino group, an ethylamino group, a dimethylamino group, a propylamino group, a butylamino group, a pentylamino group, a pentylamino group and a hexylamino group. The number of carbon atoms of the alkylamino group may be 1 to 27, is preferably 1 to 11, is more preferably 1 to 6, is further preferably 1 to 4, and is further more preferably 1 to 3.

Examples of the alkyl peptide group include an alkyl peptide group having 1 to 39 carbon atoms, and examples thereof include a methyl peptide group, an ethyl peptide group and a propyl peptide group. The number of carbon atoms of the alkyl peptide group may be 1 to 27, is preferably 1 to 11, is more preferably 1 to 6, is further preferably 1 to 4, and is further more preferably 1 to 3.

Examples of the alkoxycarbonyl group include an alkoxycarbonyl group having 2 to 39 carbon atoms, and examples thereof include a methoxycarbonyl group, an ethoxycarbonyl group and a butoxycarbonyl group. Examples of the alkylcarbonyl group include an alkylcarbonyl group having 2 to 40 carbon atoms, and examples thereof include an acetyl group, a propionyl group and a butyryl group. Examples of the alkylcarbonyloxy group include an alkylcarbonyloxy group having 2 to 39 carbon atoms, and examples thereof include an acetyloxy group, a propionyloxy group and a butyryloxy group. Examples of the alkoxycarbonyloxy group include an alkoxycarbonyloxy group having 2 to 38 carbon atoms, and examples thereof include a butoxycarbonyloxy group. Examples of the alkoxyalkoxy group include an alkoxyalkoxy group having 2 to 38 carbon atoms, and examples thereof include a methoxymethoxy group, a methoxyethoxy group, an ethoxymethoxy group and an ethoxyethoxy group. The number of carbon atoms of the alkoxycarbonyl group may be 2 to 27, is preferably 2 to 11, is more preferably 2 to 6, is further preferably 2 to 4, and is further more preferably 2 or 3. The number of carbon atoms of the alkylcarbonyl group may be 2 to 28, is preferably 2 to 12, is more preferably 2 to 6, is further preferably 2 to 4, and is further more preferably 2 or 3. The number of carbon atoms of the alkylcarbonyloxy group may be 2 to 27, is preferably 2 to 11, is more preferably 2 to 6, is further preferably 2 to 4, and is further more preferably 2 or 3. The number of carbon atoms of the alkoxycarbonyloxy group may be 2 to 26, is preferably 2 to 10, is more preferably 2 to 6, is further preferably 2 to 4, and is further more preferably 2 or 3. The number of carbon atoms of the alkoxyalkoxy group may be 2 to 26, is preferably 2 to 10, is more preferably 2 to 6, is further preferably 2 to 4, and is further more preferably 2 or 3.

Examples of the alkoxyalkyl group include an alkoxyalkyl group having 2 to 39 carbon atoms, and examples thereof include a methoxymethyl group, a methoxyethyl group, an ethoxymethyl group, an ethoxyethyl group, an ethoxypropyl group, an ethoxybutyl group and a propoxymethyl group. The number of carbon atoms of the alkoxyalkyl group may be 2 to 27, is preferably 2 to 11, is more preferably 2 to 6, is further preferably 2 to 4, and is further more preferably 2 or 3.

Examples of the hydroxyalkyl group include a hydroxyalkyl group having 1 to 39 carbon atoms, and examples thereof include a hydroxymethyl group, a dihydroxymethyl group, a hydroxyethyl group, a dihydroxyethyl group and a hydroxypropyl group. The number of carbon atoms of the hydroxyalkyl group may be 1 to 27, is preferably 1 to 11, is more preferably 1 to 6, is further preferably 1 to 4, and is further more preferably 1 to 3.

Examples of the alkanediyloxy group include an alkanediyloxy group having 1 to 39 carbon atoms, and examples thereof include a methyleneoxy group, an ethyleneoxy group, a propanediyloxy group, a butanediyloxy group and a pentanediyloxy group. The number of carbon atoms of the alkanediyloxy group may be 1 to 27, is preferably 1 to 11, is more preferably 1 to 6, is further preferably 1 to 4, and is further more preferably 1 to 3.

Examples of the alkanediyloxycarbonyl group include an alkanediyloxycarbonyl group having 2 to 39 carbon atoms, and examples thereof include a methyleneoxycarbonyl group, an ethyleneoxycarbonyl group, a propanediyloxycarbonyl group and a butanediyloxycarbonyl group. Examples of the alkanediyl carbonyl group include an alkanediylcarbonyl group having 2 to 40 carbon atoms, and examples thereof include a methylenecarbonyl group, an ethylenecarbonyl group, a propanediylcarbonyl group, a butanediylcarbonyl group and a pentanediylcarbonyl group. Examples of the alkanediylcarbonyloxy group include an alkanediylcarbonyloxy group having 2 to 39 carbon atoms, and examples thereof include a methylenecarbonyloxy group, an ethylenecarbonyloxy group, a propanediylcarbonyloxy group and a butanediylcarbonyloxy group. Examples of the alkanediyloxycarbonyloxy group include an alkoxycarbonyloxy group having 2 to 38 carbon atoms, and examples thereof include a butoxycarbonyloxy group. The number of carbon atoms of the alkanediyloxycarbonyl group may be 2 to 27, is preferably 2 to 11, is more preferably 2 to 6, is further preferably 2 to 4, and is further more preferably 2 or 3. The number of carbon atoms of the alkanediylcarbonyl group may be 2 to 28, is preferably 2 to 12, is more preferably 2 to 6, is further preferably 2 to 4, and is further more preferably 2 or 3. The number of carbon atoms of the alkanediylcarbonyloxy group may be 2 to 27, is preferably 2 to 11, is more preferably 2 to 6, is further preferably 2 to 4, and is further more preferably 2 or 3. The number of carbon atoms of the alkanediyloxycarbonyloxy group may be 2 to 26, is preferably 2 to 10, is more preferably 2 to 6, is further preferably 2 to 4, and is further more preferably 2 or 3.

Examples of the alkanediylsulfonyl group include an alkanediylsulfonyl group having 1 to 39 carbon atoms, and examples thereof include a methylenesulfonyl group, an ethylenesulfonyl group and a propylenesulfonyl group. The number of carbon atoms of the alkanediylsulfonyl group may be 1 to 27, is preferably 1 to 11, is more preferably 1 to 6, is further preferably 1 to 4, and is further more preferably 1 to 3.

Examples of the alkanediylthio group include an alkanediylthio group having 1 to 39 carbon atoms, and examples thereof include a methylenethio group, an ethylenethio group and a propylenethio group. The number of carbon atoms of the alkanediylthio group may be 1 to 27, is preferably 1 to 11, is more preferably 1 to 6, is further preferably 1 to 4, and is further more preferably 1 to 3.

Examples of the alkanediylamino group include an alkanediylamino group having 1 to 39 carbon atoms, and examples thereof include a methyleneamino group, an ethyleneamino group, a dimethyleneamino group, a propyleneamino group, a butyleneamino group, a pentyleneamino group and a hexyleneamino group. The number of carbon atoms of the alkanediylamino group may be 1 to 27, is preferably 1 to 11, is more preferably 1 to 6, is further preferably 1 to 4, and is further more preferably 1 to 3.

Examples of the alkanediyl peptide group include an alkanediyl peptide group having 1 to 39 carbon atoms, and the examples thereof include a methylene peptide group, an ethylene peptide group and a propylene peptide group. The number of carbon atoms of the alkanediyl peptide group may be 1 to 27, is preferably 1 to 11, is more preferably 1 to 6, is further preferably 1 to 4, and is further more preferably 1 to 3.

Examples of the cycloalkoxy group include a cycloalkoxy group having 3 to 39 carbon atoms, and examples thereof include a cyclohexyloxy group. Examples of the cycloalkylalkoxy group include a cycloalkylalkoxy group having 4 to 39 carbon atoms, and examples thereof include a cyclohexylmethoxy group. Examples of the aromatic hydrocarbon group-carbonyloxy group include an aromatic hydrocarbon group -carbonyloxy group having 7 to 39 carbon atoms, and examples thereof include a benzoyloxy group. Examples of the aromatic hydrocarbon group-carbonyl group include an aromatic hydrocarbon group-carbonyl group having 7 to 40 carbon atoms, and examples thereof include a benzoyl group. Examples of the aromatic hydrocarbon group-oxy group include an aromatic hydrocarbon group-oxy group having 6 to 39 carbon atoms, and examples thereof include a phenyloxy group.

In addition, examples of the group formed by replacing —CH₂-contained in the alicyclic hydrocarbon group, the aromatic hydrocarbon group, or the combined group of these groups with —O—, —S—, —CO—, —SO—, —NR⁷— or —SO₂-include the following groups. In the groups shown below, —O— and —CO— are each optionally replaced with —S—, —NR⁷—, —SO— or —SO₂—. The bonding site can be set to any position.

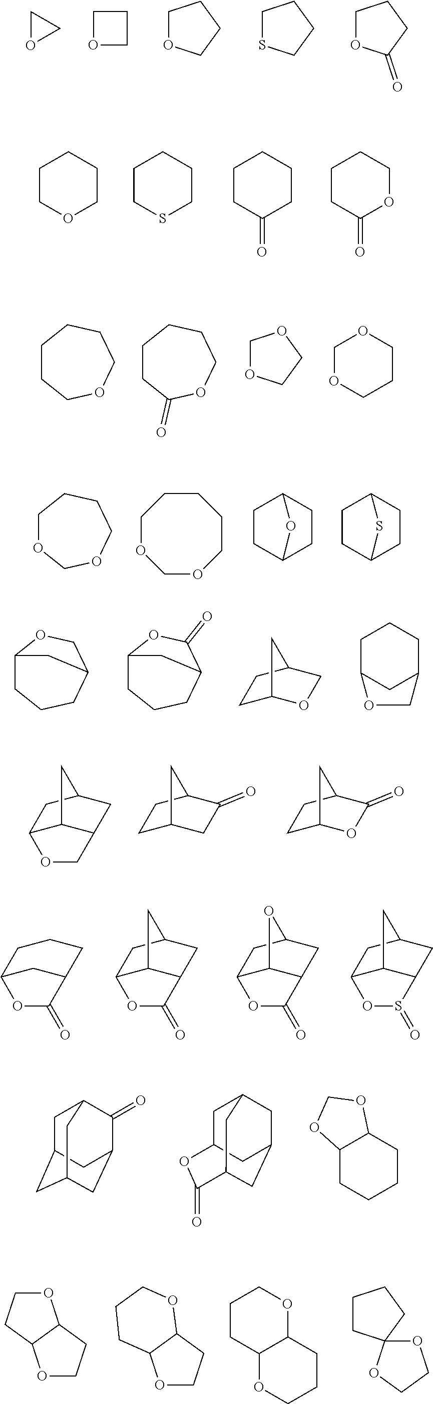

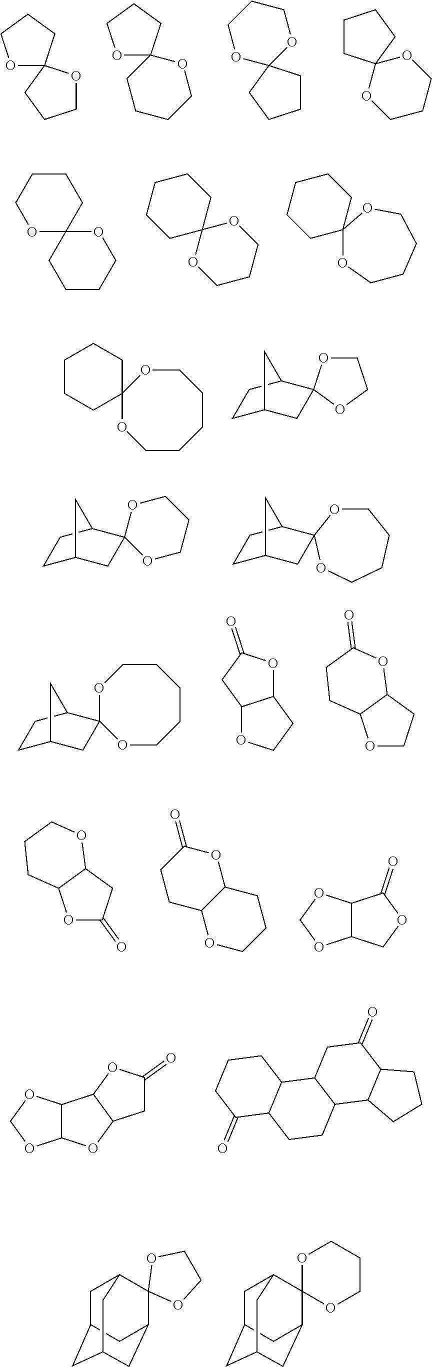

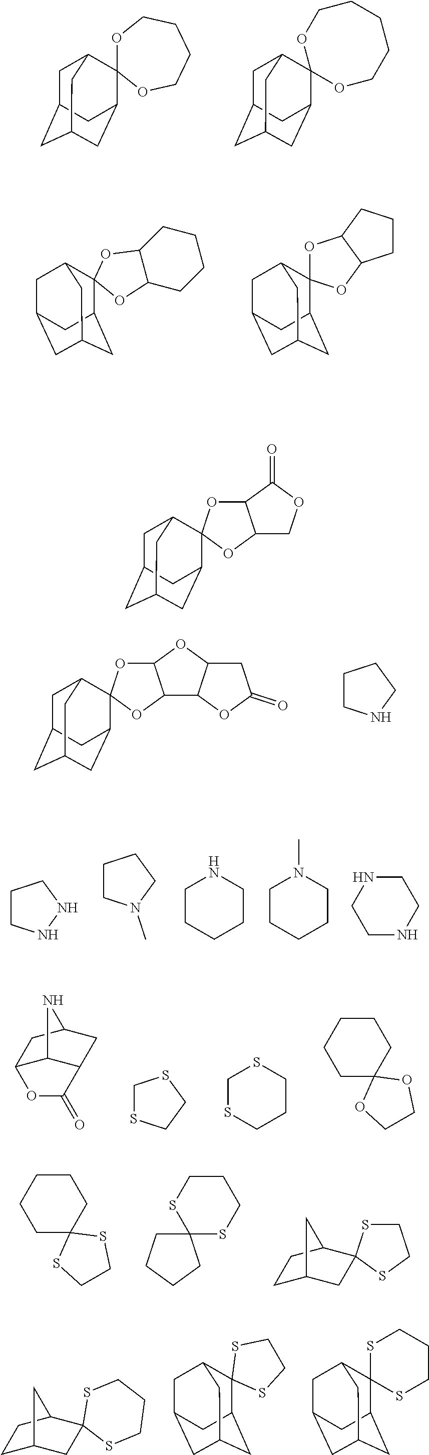

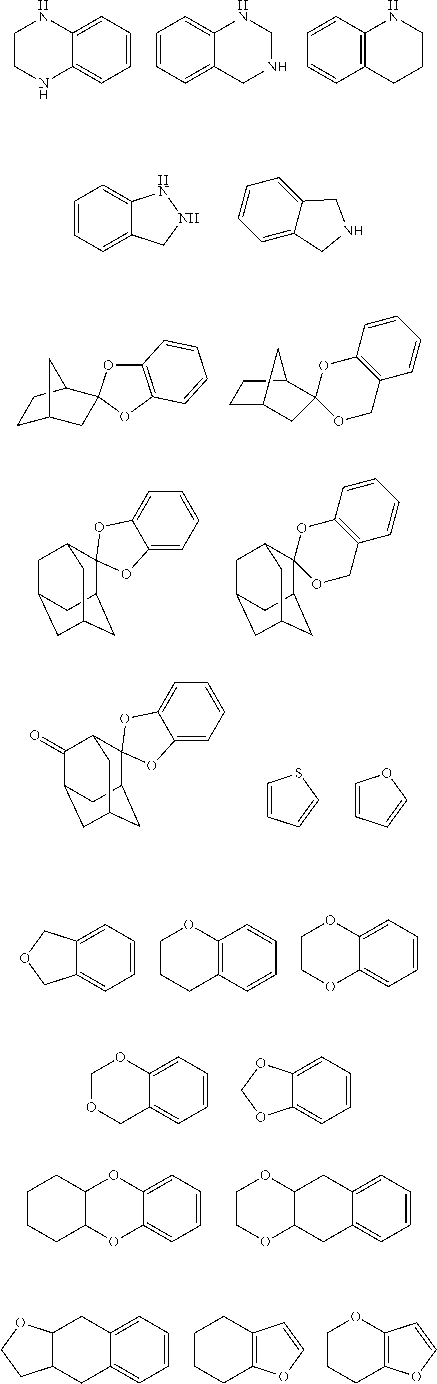

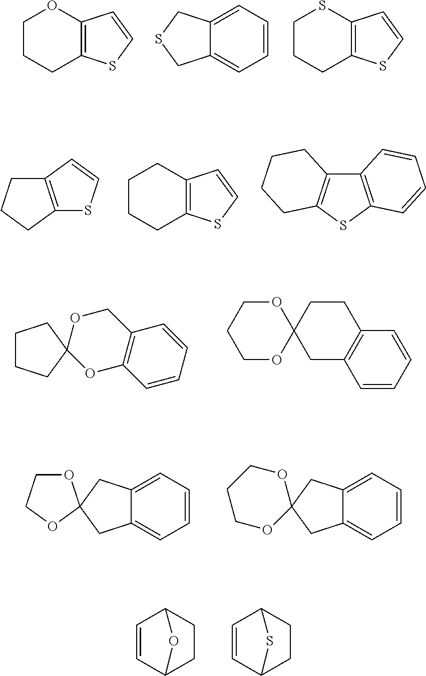

Examples of the substituent that L¹ optionally has include a halogen atom, a cyano group and a nitro group. Due to the replacement of —CH₂-contained in the chain hydrocarbon group of L¹, such as alkyl group, with —O—, —S—, —CO—, —NR⁷—, —SO— or —SO₂—, L¹ can substantially have a substituent such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group and an alkyl peptide group. In addition, when L¹ is a combined group of an alicyclic hydrocarbon group or an aromatic hydrocarbon group and a chain hydrocarbon group such as alkyl group, the chain hydrocarbon group such as alkyl group can substantially be a substituent for the alicyclic hydrocarbon group or the aromatic hydrocarbon group.

Examples of the halogen atom include a fluorine atom, a chlorine atom, a bromine atom and an iodine atom.

Examples of L¹ include a single bond, a chain hydrocarbon group having 1 to 18 carbon atoms and optionally having a substituent, where —CH₂-contained in the chain hydrocarbon group is optionally replaced with —O—, —CO—, —S—, —SO—, —SO₂— or —NR⁷—, a cyclic hydrocarbon group having 3 to 18 carbon atoms and optionally having a substituent, where —CH₂— contained in the cyclic hydrocarbon group is optionally replaced with —O—, —CO—, —S—, —SO—, —SO₂— or NR⁷—, or a group formed by combining these groups, and it is preferably a single bond, a chain hydrocarbon group having 1 to 12 carbon atoms and optionally having a substituent, where —CH₂— contained in the chain hydrocarbon group is optionally replaced with —O—, —S—, —SO₂—, —SO—, —NR⁷— or —CO—, a cyclic hydrocarbon group having 3 to 18 carbon atoms and optionally having a substituent, where —CH₂— contained in the cyclic hydrocarbon group is optionally replaced with —O—, —S—, —SO₂—, —SO—, —NR⁷— or —CO—, or a combined group of a chain hydrocarbon group having 1 to 10 carbon atoms and optionally having a substituent and a cyclic hydrocarbon group having 3 to 18 carbon atoms and optionally having a substituent, where —CH₂— contained in the chain hydrocarbon group and the cyclic hydrocarbon group is optionally replaced with —O—, —S—, —SO₂—, —SO—, —NR⁷— or —CO—, is more preferably a single bond, a chain hydrocarbon group having 1 to 10 carbon atoms, where —CH₂— contained in the chain hydrocarbon group is optionally replaced with —O—, —S—, —SO₂—, —SO—, —NR⁷— or —CO—, or a combined group of a chain hydrocarbon group having 1 to 6 carbon atoms and a cyclic hydrocarbon group having 3 to 12 carbon atoms and optionally having a substituent, where —CH₂— contained in the chain hydrocarbon group and the cyclic hydrocarbon group is optionally replaced with —O—, —S—, —SO₂—, —SO—, —NR⁷— or —CO—, is further preferably a single bond, a chain hydrocarbon group having 1 to 8 carbon atoms, where —CH₂— contained in the chain hydrocarbon group is optionally replaced with —O— or —CO—, a combined group of a chain hydrocarbon group having 1 to 4 carbon atoms and an alicyclic hydrocarbon group having 3 to 12 carbon atoms and optionally having a substituent, where —CH₂— contained in the chain hydrocarbon group and the alicyclic hydrocarbon group is optionally replaced with —O— or —CO—, or a combined group of a chain hydrocarbon group having 1 to 4 carbon atoms and an aromatic hydrocarbon group having 6 to 10 carbon atoms and optionally having a substituent, where —CH₂— contained in the chain hydrocarbon group is optionally replaced with —O— or —CO—, and is further more preferably a single bond, a chain hydrocarbon group having 1 to 6 carbon atoms, where —CH₂-contained in the chain hydrocarbon group is optionally replaced with —O— or —CO—, a combined group of a chain hydrocarbon group having 1 to 4 carbon atoms and an alicyclic hydrocarbon group having 3 to 12 carbon atoms, where —CH₂— contained in the chain hydrocarbon group is optionally replaced with —O— or —CO—, or a combined group of a chain hydrocarbon group having 1 to 4 carbon atoms and an aromatic hydrocarbon group having 6 to 10 carbon atoms, where —CH₂— contained in the chain hydrocarbon group is optionally replaced with —O— or —CO—.

In formula (I), the cyclic hydrocarbon group having 3 to 36 carbon atoms of W¹ (hereinafter referred to as “ring W¹” in some cases) may be either saturated or unsaturated, may be monocyclic or polycyclic, and the polycyclic cyclic hydrocarbon group may be a bridged ring or may form a spiro. Examples of the cyclic hydrocarbon group include an alicyclic hydrocarbon group, an aromatic hydrocarbon group, or a combined group of these groups. The carbon atoms contained in the alicyclic hydrocarbon group may form a double bond between two adjacent carbon atoms. The cyclic hydrocarbon group of W¹ is a cyclic hydrocarbon group having a valence of (mm2+1), and depending on mm2, it can be a divalent to hexavalent cyclic hydrocarbon group. The number of carbon atoms of the cyclic hydrocarbon group is preferably 3 to 30, is more preferably 3 to 24, and is further preferably 3 to 20.

Examples of the alicyclic hydrocarbon group and the cyclic hydrocarbon group in which a double bond is formed between two adjacent carbon atoms contained in an alicyclic hydrocarbon group and alicyclic hydrocarbon groups include the following alicyclic hydrocarbon groups. The bonding site can be set to any position.

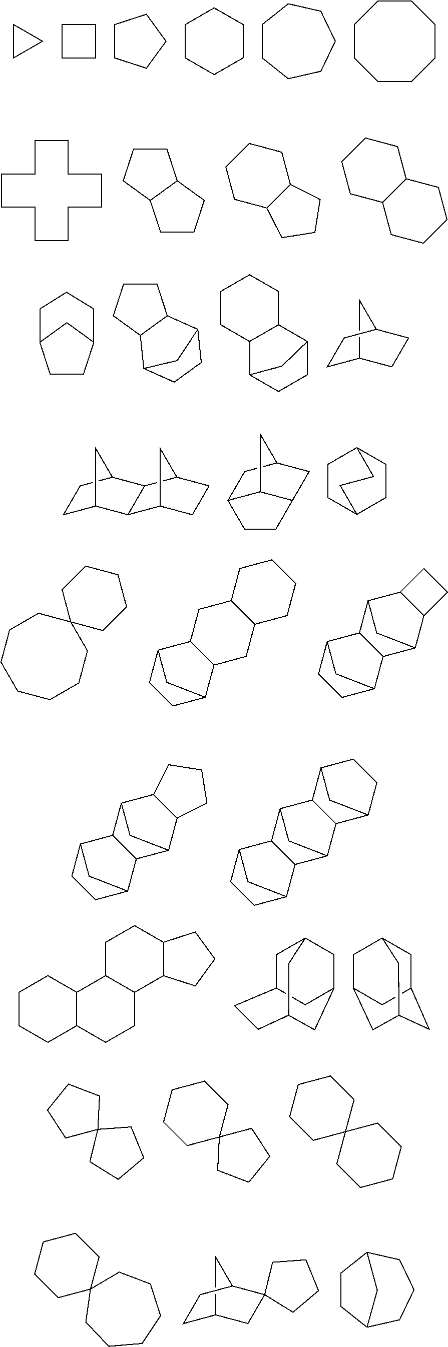

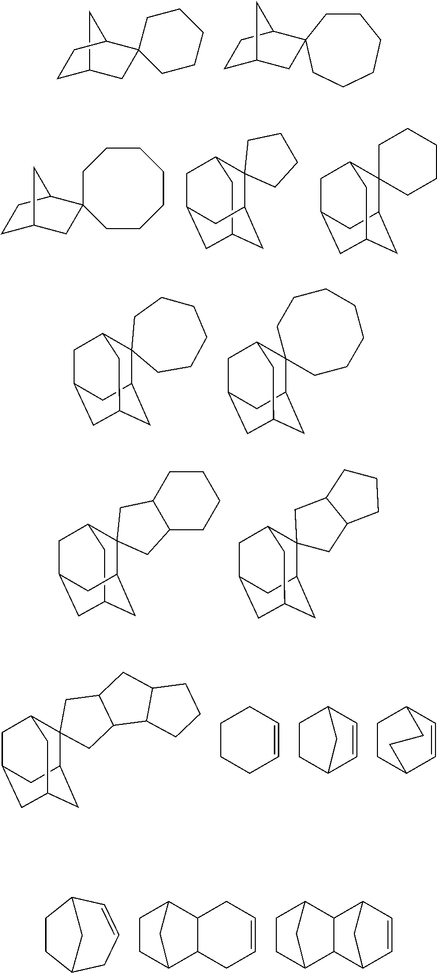

Specific example of the monocyclic alicyclic hydrocarbon group include a monocyclic cycloalkanediyl group such as a cyclopropanediyl group, a cyclobutanediyl group, a cyclopentanediyl group, a cyclohexanediyl group, a cycloheptanediyl group, a cyclooctanediyl group and a cyclodecanediyl group. Examples of the polycyclic alicyclic hydrocarbon group include a polycyclic cycloalkanediyl group such as a decahydronaphthalenediyl group, an adamantanediyl group, a norbornanediyl group, a norbornenediyl group and a bicyclo[3.3.0]octanediyl group.

Examples thereof also include a group in which one or more hydrogen atoms of the above groups are replaced with bonding sites.

The number of carbon atoms of the alicyclic hydrocarbon group may be 3 to 30, is preferably 3 to 24, is more preferably 3 to 18, is further preferably 3 to 16, and is further more preferably 3 to 12.

Examples of the aromatic hydrocarbon group include an aromatic hydrocarbon group such as an arylene group including a phenylene group, a naphthylene group, an anthrylene group, a biphenylene group and a phenanthrylene group. Specific examples thereof include the following aromatic hydrocarbon groups.

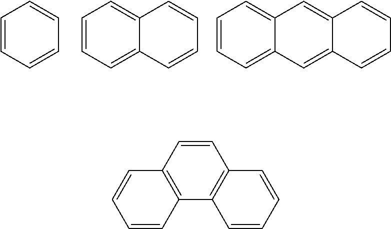

In addition, examples thereof also include a group in which one or more hydrogen atoms of the above groups are replaced with bonding sites.

The number of carbon atoms of the aromatic hydrocarbon group is preferably 6 to 24, is more preferably 6 to 18, is further preferably 6 to 14, and is further more preferably 6 to 10.

Examples of the group formed by replacing —CH₂— contained in the alicyclic hydrocarbon group, the aromatic hydrocarbon group, or the combined group of these groups with —O—, —S—, —CO—, —SO—, —NR⁷— or —SO₂— include the following groups, and as long as permitted by the upper limit of the number of carbon atoms, groups similar to those described above can be mentioned. The cyclic hydrocarbon group is preferably a cyclic hydrocarbon group having 3 to 18 carbon atoms, where a hydrogen atom contained in the cyclic hydrocarbon group is optionally replaced with a substituent and —CH₂— contained in the cyclic hydrocarbon group is optionally replaced with —O—, —S—, —CO—, —SO—, —NR⁷— or —SO₂—, is more preferably an alicyclic hydrocarbon group having 3 to 18 carbon atoms or an aromatic hydrocarbon group having 6 to 18 carbon atoms, where a hydrogen atom contained in the alicyclic hydrocarbon group and the aromatic hydrocarbon group is optionally replaced with a substituent and —CH₂— contained in the alicyclic hydrocarbon group is optionally replaced with —O—, —S—, —CO—, —SO—, —NR⁷— or —SO₂—, and is further preferably a group represented by any one of formulas (w1-1) to (w1-33):

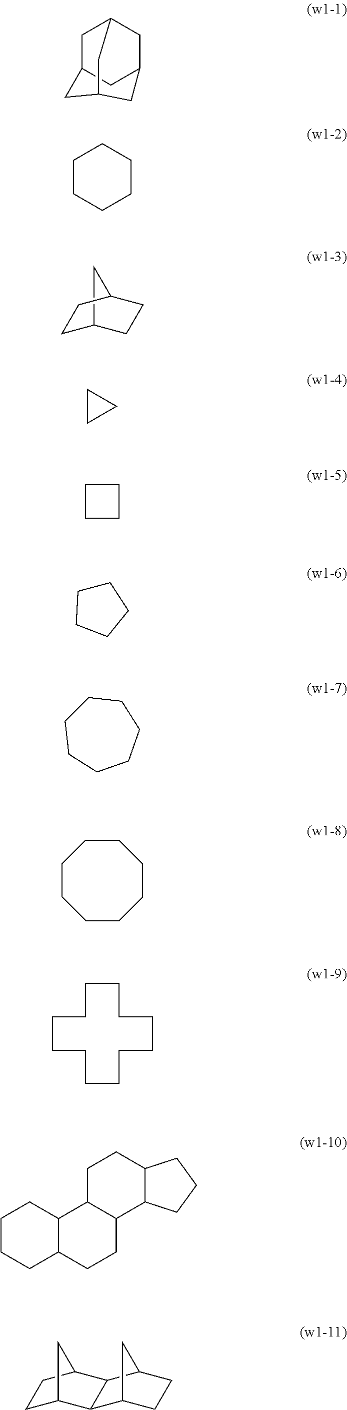

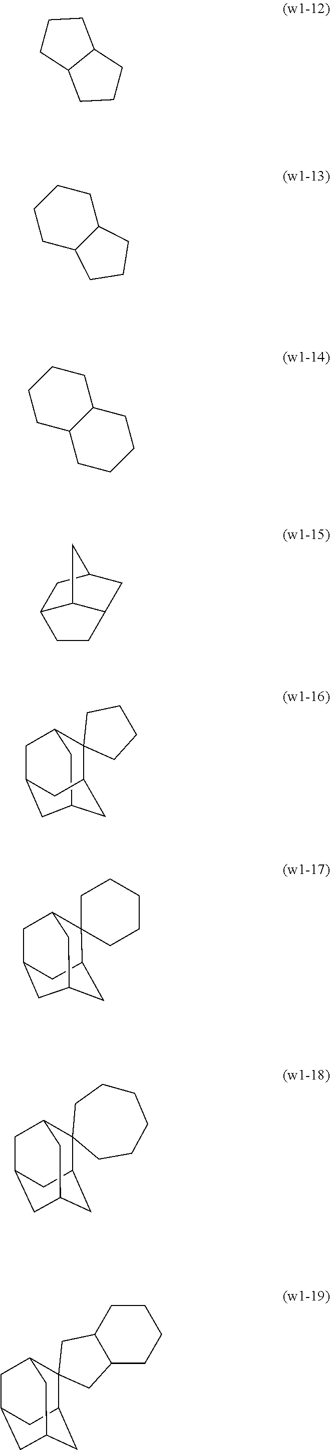

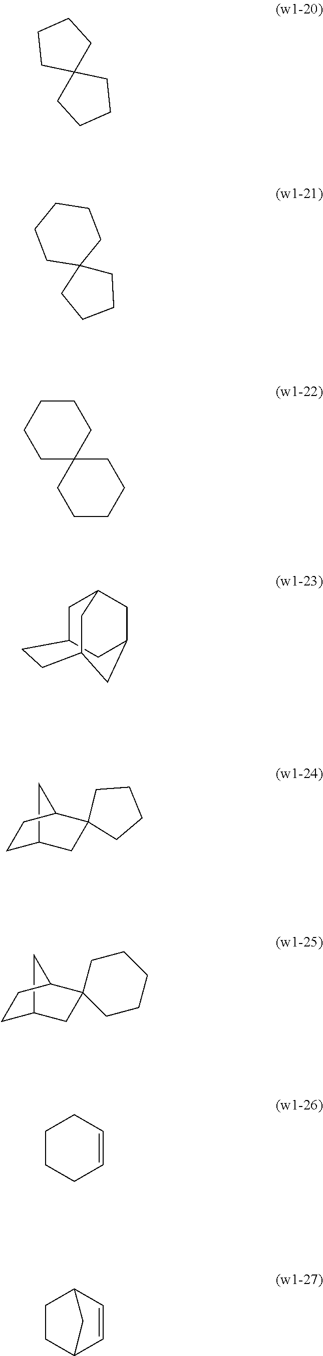

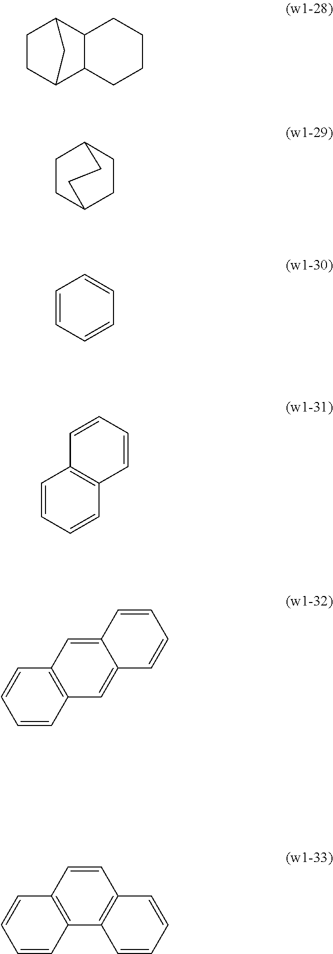

wherein

a hydrogen atom contained in the group is optionally replaced with a substituent, and —CH₂— contained in the group is optionally replaced with —O—, —S—, —CO—, —SO—, —NR⁷— or —SO₂—; and the bonding site is set to any position.

Examples of the substituent of the cyclic hydrocarbon group contained in W¹ include a halogen atom, a cyano group, a nitro group, a haloalkyl group having 1 to 6 carbon atoms, and an alkyl group having 1 to 12 carbon atoms, where —CH₂— contained in the alkyl group is optionally replaced with —O—, —CO—, —S—, —SO—, —NR⁷— or —SO₂—.

Examples of the halogen atom described above include a fluorine atom, a chlorine atom, a bromine atom and an iodine atom.

Examples of the haloalkyl group having 1 to 6 carbon atoms described above include a fluorinated alkyl group having 1 to 6 carbon atoms, a chlorinated alkyl group having 1 to 6 carbon atoms, a brominated alkyl group having 1 to 6 carbon atoms, and an iodinated alkyl group having 1 to 6 carbon atoms. Examples of the haloalkyl group include a perfluoroalkyl group having 1 to 6 carbon atoms (such as a trifluoromethyl group, a pentafluoroethyl group, a heptafluoropropyl group and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group, a fluoromethyl group and a difluoromethyl group. The number of carbon atoms of the haloalkyl group is preferably 1 to 4, and is more preferably 1 to 3.

Examples of the alkyl group having 1 to 12 carbon atoms described above include an alkyl group such as a methyl group, an ethyl group, a propyl group, an isopropyl group, a butyl group, an isobutyl group, a tert-butyl group, a pentyl group, a hexyl group, an octyl group and a nonyl group. The number of carbon atoms of the alkyl group is preferably 1 to 9, is more preferably 1 to 6, and is further preferably 1 to 4.

When —CH₂— contained in the alkyl group described above is replaced with —O—, —CO—, —S—, —SO—, —NR⁷— or —SO₂—, the number of carbon atoms before the replacement is regarded as the total number of carbon atoms of the alkyl group.

Examples of the group formed by the replacement include a hydroxy group, a carboxy group, an oxy group, a carbonyl group, an alkoxy group, an alkoxycarbonyl group, an alkylcarbonyl group, an alkylcarbonyloxy group, a thiol group, an alkylthio group, an alkylsulfonyl group, an alkoxycarbonyloxy group, an alkoxyalkoxy group, an amino group, an alkylamino group, an alkyl peptide group, and a combined group of two or more of these groups. Examples of these groups formed by the replacement include the same groups as those exemplified in the present specification, within the range permitted by the upper limit of the number of carbon atoms.

Examples of the alkoxy group include an alkoxy group having 1 to 11 carbon atoms. Examples of the alkoxycarbonyl group include an alkoxycarbonyl group having 2 to 11 carbon atoms. Examples of the alkylcarbonyl group include an alkylcarbonyl group having 2 to 12 carbon atoms. Examples of the alkylcarbonyloxy group include an alkylcarbonyloxy group having 2 to 11 carbon atoms. Examples of the alkylthio group include an alkylthio group having 1 to 11 carbon atoms. Examples of the alkylsulfonyl group include an alkylsulfonyl group having 1 to 11 carbon atoms. Examples of the alkoxycarbonyloxy group include an alkoxycarbonyloxy group having 1 to 10 carbon atoms. Examples of the alkoxyalkoxy group include an alkoxyalkoxy group having 1 to 10 carbon atoms. Examples of the alkylamino group include an alkylamino group having 1 to 11 carbon atoms. Examples of the alkyl peptide group include an alkyl peptide group having 1 to 11 carbon atoms. Examples of these groups formed by the replacement include the same groups as those exemplified in the present specification, within the range permitted by the upper limit of the number of carbon atoms.

The substituent that W¹ optionally has is preferably a halogen atom, a haloalkyl group having 1 to 3 carbon atoms or an alkyl group having 1 to 6 carbon atoms, where —CH₂— contained in the alkyl group is optionally replaced with —O— or —CO—, is more preferably a fluorine atom, an iodine atom, a fluorinated alkyl group having 1 to 3 carbon atoms, an alkyl group having 1 to 3 carbon atoms, a hydroxy group, an alkoxy group having 1 to 3 carbon atoms or a carboxy group, is further preferably a fluorine atom, an iodine atom, a trifluoromethyl group, a methyl group, an ethyl group, a hydroxy group, a methoxy group, an ethoxy group or a carboxy group, and is further more preferably a hydroxy group, a methoxy group, a fluorine atom or an iodine atom.

The number of substituents that W¹ optionally has is not particularly limited, but is preferably 1, 2 or 4.

W¹ is preferably a single bond or a cyclic hydrocarbon group having 3 to 18 carbon atoms, where a hydrogen atom contained in the cyclic hydrocarbon group is optionally replaced with a substituent and —CH₂— contained in the cyclic hydrocarbon group is optionally replaced with —O—, —S—, —CO—, —SO—, —NR⁷— or —SO₂—, is more preferably a single bond or a cyclic hydrocarbon group represented by formula (W-0) (hereinafter referred to as “ring W-0” in some cases), and is further preferably a single bond or a cyclic hydrocarbon group represented by formula (W-1) (hereinafter referred to as “ring W-1” in some cases):

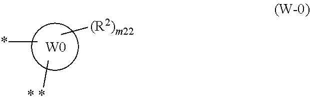

wherein
W0 represents a cyclic hydrocarbon group having 3 to 10 carbon atoms, the carbon atoms contained in the cyclic hydrocarbon group may form a double bond between two adjacent carbon atoms, —CH₂— contained in the cyclic hydrocarbon group is optionally replaced with —O—, —S—, —CO—, —SO—, —NR⁷— or —SO₂—, and a hydrogen atom contained in the cyclic hydrocarbon group is optionally replaced with a substituent;
R²² represents a halogen atom, a haloalkyl group having 1 to 6 carbon atoms or an alkyl group having 1 to 12 carbon atoms, —CH₂— contained in the alkyl group is optionally replaced with —O—, —CO—, —S—, —SO—, —NR⁷— or —SO₂—, the alkyl group may be bonded to a carbon atom contained in W¹ to form an alkanediyl bridge and —CH₂— contained in the alkanediyl bridge is optionally replaced with —O—, —CO—, —S—, —SO—, —NR⁷— or —SO₂—, the carbon atoms contained in the alkanediyl bridge may form a double bond between two adjacent carbon atoms, and the alkanediyl bridge optionally has a substituent;
m22 represents an integer of 0 to 5, and when m22 is 2 or larger, a plurality of R²² may be the same or different from each other; and
* represents a bonding site to L¹, and ** represents a bonding site to A¹,

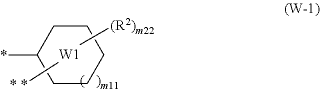

wherein
W1 represents a cyclic hydrocarbon group having (5+m11) carbon atoms, the carbon atoms contained in the cyclic hydrocarbon group may form a double bond between two adjacent carbon atoms, —CH₂— contained in the cyclic hydrocarbon group is optionally replaced with —O—, —S—, —CO—, —SO—, —NR⁷— or —SO₂—, and a hydrogen atom contained in the cyclic hydrocarbon group is optionally replaced with a substituent;
R²² represents a halogen atom, a haloalkyl group having 1 to 6 carbon atoms or an alkyl group having 1 to 12 carbon atoms, —CH₂— contained in the alkyl group is optionally replaced with —O—, —CO—, —S—, —SO—, —NR⁷— or —SO₂—, the alkyl group may be bonded to a carbon atom contained in W¹ to form an alkanediyl bridge and —CH₂— contained in the alkanediyl bridge is optionally replaced with —O—, —CO—, —S—, —SO—, —NR⁷— or —SO₂—, the carbon atoms contained in the alkanediyl bridge may form a double bond between two adjacent carbon atoms, and the alkanediyl bridge optionally has a substituent;
m11 represents an integer of 0 to 3;
m22 represents an integer of 0 to 5, and when m22 is 2 or larger, a plurality of R²² may be the same or different from each other; and
* represents a bonding site to L¹, and ** represents a bonding site to A¹.

In the ring W-0 or the ring W-1, the carbon atoms contained in the ring W-0 or the ring W-1 may form a double bond between two adjacent carbon atoms. Here, the number of double bonds may be one, two, or three or more, and an aromatic ring may be constituted.

Examples of the cyclic hydrocarbon group having 3 to 10 carbon atoms of W0 or the cyclic hydrocarbon group having (5+m11) carbon atoms ((5+m11)-membered ring) of W1 include groups similar to those described above as long as permitted by the respective upper limits of the number of carbon atoms.

The group in which the alkyl group of R²² is bonded to a carbon atom contained in W¹ to form an alkanediyl bridge is a straight-chain or branched alkanediyl group, and examples thereof include a methylene group, an ethylene group, a propanediyl group, a butanediyl group, a pentanediyl group, a hexanediyl group and a 2-methylene-propanediyl group.

The alkanediyl group is bonded to two carbon atoms contained in W¹, and in this case, it may be bonded to carbon atoms constituting the cyclic hydrocarbon group of W0 in the ring W-0 or W1 in the ring W-1 to form an alkanediyl bridge, or there may be two or more alkanediyl groups, where a carbon atom constituting one alkanediyl group is bonded to a carbon atom constituting another alkanediyl group to form an alkanediyl bridge. The substituent described above may be bonded to the alkanediyl bridge. When —CH₂— contained in the alkanediyl group is replaced with —O—, —CO—, —S—, —SO—, —NR⁷— or —SO₂—, it is preferably —O— or —S—.
m11 is preferably an integer of 0 to 2, is more preferably 0 or 1, and is further preferably 1.
m22 is preferably an integer of 0 to 4, is more preferably 0, 1, 2 or 4, is further preferably 0, 1 or 4, and is further more preferably 0 or 1.

As the cyclic hydrocarbon group represented by formula (W-0) or (W-1), the cyclic hydrocarbon groups listed below are preferable. These cyclic hydrocarbon groups optionally have the substituent of R²² described above, and preferably have 0 to 4 of them. The bonding site can be set to any position.

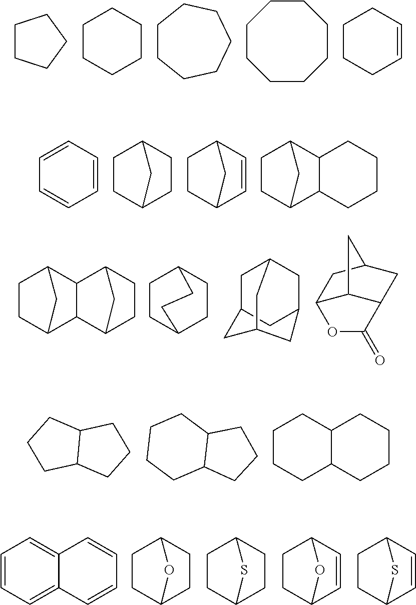

In particular, preferable are the cyclic hydrocarbon groups represented as follows.

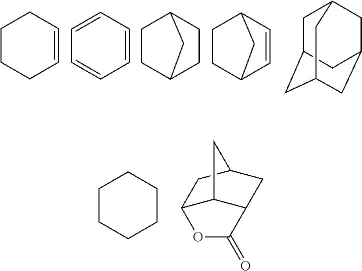

Examples of the hydrocarbon group in A¹ include a straight-chain or branched chain hydrocarbon group (such as an alkanediyl group), a monocyclic or polycyclic (including a spiro ring, a condensed ring or a bridged ring), saturated or unsaturated alicyclic hydrocarbon group, and an aromatic hydrocarbon group, and it may be a combined group of two or more of these groups (for example, a hydrocarbon group formed from an alicyclic hydrocarbon group or an aromatic hydrocarbon group and a chain hydrocarbon group). The number of carbon atoms of the hydrocarbon group is preferably 1 to 19, is more preferably 1 to 18, is further preferably 1 to 16, is still more preferably 1 to 14, and is still further preferably 1 to 12.

Examples of the chain hydrocarbon group include: a straight-chain alkanediyl group such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group; and
a branched alkanediyl group such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The terminal of the branched alkanediyl group may be an alkyl group such as a methyl group.

The number of carbon atoms of the chain hydrocarbon group is preferably 1 to 18, is more preferably 1 to 12, is further preferably 1 to 10, is further more preferably 1 to 9, is even more preferably 1 to 8, is still more preferably 1 to 6, is still further preferably 1 to 4, and is further more preferably 1 to 3.

The alicyclic hydrocarbon group may be either monocyclic or polycyclic, and examples thereof include the groups represented as follows.

The bonding site can be set to any position.

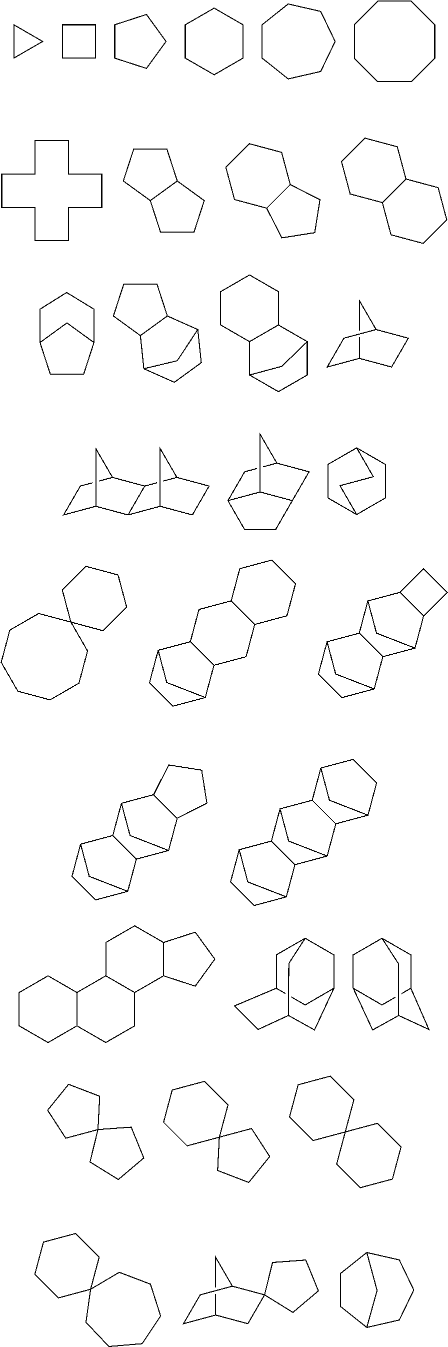

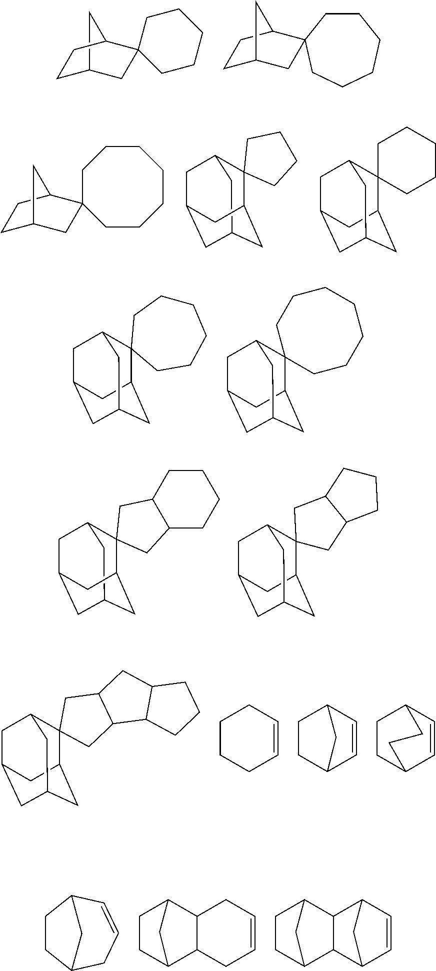

Specific examples thereof include: a monocyclic alicyclic hydrocarbon group that is a monocyclic cycloalkanediyl group such as a cyclobutane-1,3-diyl group, a cyclopentane-1,3-diyl group, a cyclohexane-1,4-diyl group and a cyclooctane-1,5-diyl group; and
a polycyclic alicyclic hydrocarbon group that is a polycyclic cycloalkanediyl group such as a spiro ring having a cycloalkyl group bonded by spiro to each of a cycloalkyl group, a norbornyl group or an adamantyl group, such as a norbornane-1,4-diyl group, a norbornane-2,5-diyl group, an adamantane-1,5-diyl group, an adamantane-2,6-diyl group, a spirocyclohexane-1,2′-cyclopentane group, or a spiroadamantane-2,3′-cyclopentane group.

The number of carbon atoms of the alicyclic hydrocarbon group is preferably 3 to 18, is more preferably 3 to 16, is further preferably 3 to 12, and is further more preferably 3 to 10.

Examples of the aromatic hydrocarbon group include an aromatic hydrocarbon group such as an arylene group including a phenylene group, a naphthylene group, an anthrylene group, a biphenylene group and a phenanthrylene group. The number of carbon atoms of the aromatic hydrocarbon group is preferably 6 to 18, is more preferably 6 to 14, and is further preferably 6 to 10.

Examples of the group formed by combining two or more groups include a combined group of an alicyclic hydrocarbon group and a chain hydrocarbon group, a combined group of an aromatic hydrocarbon group and a chain hydrocarbon group, and a combined group of an alicyclic hydrocarbon group and an aromatic hydrocarbon group. In the combination, two or more types of chain hydrocarbon groups, alicyclic hydrocarbon groups, and aromatic hydrocarbon groups may each be combined. In addition, any of the groups may be bonded to W¹.

Examples of the combined group of an alicyclic hydrocarbon group and a chain hydrocarbon group include -(divalent alicyclic hydrocarbon group)-(alkanediyl group)-, -(alkanediyl group)-(divalent alicyclic hydrocarbon group)-(alkanediyl group)-, and -(alkanediyl group)-(divalent alicyclic hydrocarbon group)-.

Examples of the combined group of an aromatic hydrocarbon group and a chain hydrocarbon group include -(divalent aromatic hydrocarbon group)-(alkanediyl group)-, -(alkanediyl group)-(divalent aromatic hydrocarbon group)-(alkanediyl group)-, and -(alkanediyl group)-(divalent aromatic hydrocarbon group)-.

Examples of the combined group of an alicyclic hydrocarbon group and an aromatic hydrocarbon group include -(aromatic hydrocarbon group)-(alicyclic hydrocarbon group)-, -(alicyclic hydrocarbon group)-(aromatic hydrocarbon group)-, and -(alicyclic hydrocarbon group)-(aromatic hydrocarbon group)-(alicyclic hydrocarbon group)-.
—CH₂— contained in the hydrocarbon group of A¹ is optionally replaced with —O—, —S—, —SO₂—, —SO—, —NR⁷— or —CO—.

When —CH₂— contained in the hydrocarbon group of A¹ is replaced with —O—, —S—, —SO₂—, —SO—, —NR⁷— or —CO—, the number of carbon atoms before replacement corresponds to the defined total number of carbon atoms of the hydrocarbon group. In addition, the number thereof may be one or two or more, but it is preferably one to three.

Examples of the group formed by replacing —CH₂— contained in the hydrocarbon group with —O—, —S—, —SO₂—, —SO—, —NR⁷— or —CO— include, as described above, a hydroxy group, a carboxy group, a thiol group, an amino group, an oxy group, a carbonyl group, a thio group, a sulfonyl group, a peptide group, an alkoxy group, an alkylthio group, an alkylsulfonyl group, an alkylamino group, an alkyl peptide group, an alkoxycarbonyl group, an alkylcarbonyl group, an alkylcarbonyloxy group, an alkoxycarbonyloxy group, an alkoxycarbonyloxy group, an alkoxyalkoxy group, an alkoxyalkyl group, a hydroxyalkyl group, an alkanediyloxy group, an alkanediyloxycarbonyl group, an alkanediylcarbonyl group, an alkanediylcarbonyloxy group, an alkanediyloxycarbonyloxy group, an alkanediyl sulfonyl group, an alkanediylthio group, an alkanediylamino group, an alkanediyl peptide group, a cycloalkoxy group, a cycloalkylalkoxy group, an aromatic hydrocarbon group-carbonyloxy group, an aromatic hydrocarbon group-carbonyl group, an aromatic hydrocarbon group-oxy group, an alicyclic hydrocarbon group, an aromatic hydrocarbon group, a combined group of these groups in which —CH₂— contained therein is replaced with —O—, —S—, —SO₂—, —NR⁷—, —SO— or —CO—, and a combined group of two or more of these groups. Examples of these groups formed by the replacement include the same groups as those exemplified in the present specification, within the range permitted by the number of carbon atoms.

Examples of the substituent that the hydrocarbon group of A¹ optionally has include a halogen atom, a cyano group and a nitro group. Due to the replacement of —CH₂— contained in the chain hydrocarbon group of A¹, such as alkyl group, with —O—, —S—, —SO₂—, —SO—, —NR⁷— or —CO—, A¹ can substantially have a substituent such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group and an alkyl peptide group. In addition, when A¹ is a combined group of an alicyclic hydrocarbon group or an aromatic hydrocarbon group and a chain hydrocarbon group such as alkyl group, the chain hydrocarbon group such as alkyl group can substantially be a substituent for the alicyclic hydrocarbon group or the aromatic hydrocarbon group.

Examples of the halogen atom include a fluorine atom, a chlorine atom, a bromine atom and an iodine atom.

A¹ may be a single bond, a chain hydrocarbon group having 1 to 18 carbon atoms, a cyclic hydrocarbon group having 3 to 18 carbon atoms, or a combined group of these groups, where the chain hydrocarbon group and the cyclic hydrocarbon group optionally have a substituent, and —CH₂— contained in the chain hydrocarbon group and the cyclic hydrocarbon group is optionally replaced with —O—, —S—, —SO₂—, —SO—, —NR⁷— or —CO—, is preferably a single bond, a chain hydrocarbon group having 1 to 10 carbon atoms, a cyclic hydrocarbon group having 3 to 18 carbon atoms, or a combined group of these groups, where the chain hydrocarbon group and the cyclic hydrocarbon group optionally have a substituent, and —CH₂— contained in the chain hydrocarbon group and the cyclic hydrocarbon group is optionally replaced with —O—, —S—, —SO₂—, —SO—, —NR⁷— or —CO—, and is more preferably a single bond, a chain hydrocarbon group having 1 to 8 carbon atoms, where —CH₂— contained in the chain hydrocarbon group is optionally replaced with —O—, —S—, —SO₂—, —SO—, —NR⁷— or —CO—, a group formed by combining a chain hydrocarbon group having 1 to 6 carbon atoms and an alicyclic hydrocarbon group having 3 to 18 carbon atoms, where —CH₂-contained in the chain hydrocarbon group and the alicyclic hydrocarbon group is optionally replaced with —O—, —S—, —SO₂—, —SO—, —NR⁷— or —CO—, or a group formed by combining a chain hydrocarbon group having 1 to 6 carbon atoms and an aromatic hydrocarbon group having 6 to 18 carbon atoms and optionally having a substituent, where —CH₂— contained in the chain hydrocarbon group is optionally replaced with —O—, —S—, —SO₂—, —SO—, —NR⁷— or —CO—.

In addition, A¹ is also preferably a single bond, or a hydrocarbon group having 1 to 20 carbon atoms, where the hydrocarbon group optionally has a substituent, and —CH₂— contained in the hydrocarbon group is optionally replaced with —O—, —CO—, —S—, —SO—, —NR⁷— or —SO₂—, provided that at least one —CH₂-contained in the hydrocarbon group is replaced with —O—, —CO—, —S—, —SO—, —NR⁷— or —SO₂—.

In this case, for example, A¹ is preferably a single bond, or *—X^{1A}-A^{1A}-, where X^{1A} represents *—CO—O—, *—O—CO—, *—O—CO—O—, *—O—, *—CO—NR⁷—, *—NR⁷—CO—, *—NR⁷—CO—O—, *—O—CO—NR⁷— or *—NR⁷—, * represents a bonding site to W¹, A^{1A} represents a single bond, a chain hydrocarbon group having 1 to 4 carbon atoms, a cyclic hydrocarbon group having 3 to 12 carbon atoms, or a combined group of these groups, and —CH₂— contained in the chain hydrocarbon group and the cyclic hydrocarbon group is optionally replaced with —O—, —S—, —SO₂—, —SO—, —NR⁷— or —CO—.

In addition, A¹ is more preferably a single bond, or ***-L⁰¹¹-X⁰¹-L⁰¹²-, where X⁰¹ represents —O—, —CO—, —S—, —SO—, —NR⁷— or —SO₂—, L⁰¹¹ and L⁰¹² each independently represent a single bond or a hydrocarbon group having 1 to 18 carbon atoms, the hydrocarbon group optionally has a substituent, —CH₂— contained in the hydrocarbon group is optionally replaced with —O—, —CO—, —S—, —SO—, —NR⁷— or —SO₂—, provided that the total number of carbon atoms of L⁰¹¹ and L⁰¹² is 0 to 18, and *** represents a bonding site to W¹, and A¹ is further preferably a single bond, ***—X⁰¹-L⁰¹- or ***-L⁰¹-X⁰¹—, where X⁰¹ represents O—, —CO—, —S—, —SO—, —NR⁷— or —SO₂—, L⁰¹ represents a single bond or a hydrocarbon group having 1 to 18 carbon atoms, the hydrocarbon group optionally has a substituent, —CH₂— contained in the hydrocarbon group is optionally replaced with —O—, —CO—, —S—, —SO—, —NR⁷— or —SO₂—, and *** represents a bonding site to W¹. A¹ is also preferably one that has no substituent other than the replacement with —O—, —CO—, —S—, —SO—, —NR⁷— or —SO₂—.

Examples of the hydrocarbon group having 1 to 18 carbon atoms of L⁰¹¹, L⁰¹² and L⁰¹, where the hydrocarbon group optionally has a substituent, and —CH₂— contained in the hydrocarbon group is optionally replaced with —O—, —CO—, —S—, —SO—, —NR⁷— or —SO₂—, include the same groups as those listed for A¹ within the range of 1 to 18 carbon atoms.

X⁰¹ is preferably —O—, —S—, —NR⁷— or —CO—, and is more preferably —O—, —NR⁷— or —CO—.

L⁰¹¹, L⁰¹² and L⁰¹ are each independently preferably a single bond or a hydrocarbon group having 1 to 12 carbon atoms, where —CH₂— contained in the hydrocarbon group is optionally replaced with —O—, —CO—, —S—, —SO—, —NR⁷— or —SO₂—, more preferably a single bond or a chain hydrocarbon group having 1 to 9 carbon atoms, where —CH₂— contained in the chain hydrocarbon group is optionally replaced with —O—, —CO—, —S—, —SO—, —NR⁷— or —SO₂—, further preferably a single bond or an alkanediyl group having 1 to 6 carbon atoms, where —CH₂-contained in the alkanediyl group is optionally replaced with —O— or —CO—, still more preferably a single bond or an alkanediyl group having 1 to 5 carbon atoms, where —CH₂— contained in the alkanediyl group is optionally replaced with —O— or —CO—, still further preferably a single bond or an alkanediyl group having 1 to 4 carbon atoms, where —CH₂— contained in the alkanediyl group is optionally replaced with —O— or —CO—, and further more preferably a single bond or an alkanediyl group having 1 to 3 carbon atoms, where —CH₂— contained in the alkanediyl group is optionally replaced with —O— or —CO—. In particular, preferable is a single bond or *1-L^{01A}-L^{01B}-, where L^{01A} is a single bond, —O— or —CO—, L^{01B} represents a methylene group, an ethane-1,1-diyl group, a propane-1,1-diyl group, a propane-2,2-diyl group, an oxy group, a carbonyl group, a carbonyloxy group, a carbonyloxymethylene group, an ethyleneoxy group, a methylenecarbonyloxymethylene group or an ethyleneoxycarbonyl group, and *1 represents a bonding site to W¹ or X⁰¹, and more preferable is a single bond, a methylene group, an oxy group or a carbonyl group.

Examples of the cyclic hydrocarbon group of W² include a monocyclic or polycyclic (including a spiro ring, a condensed ring or a bridged ring), saturated or unsaturated cyclic hydrocarbon group, such as an alicyclic hydrocarbon group, an aromatic hydrocarbon group or a combined group of these groups. The carbon atoms contained in the alicyclic hydrocarbon group may form a double bond between two adjacent carbon atoms. Examples of the cyclic hydrocarbon group of W² include a group in which one or more hydrogen atoms of a monovalent cyclic hydrocarbon group are replaced with bonding sites depending on the number of iodine atoms and the like. The number of carbon atoms of the cyclic hydrocarbon group is preferably 3 to 30, is more preferably 3 to 24, and is further preferably 3 to 20.

Examples of the alicyclic hydrocarbon group include a monocyclic or polycyclic cycloalkyl group.

Examples of the monocyclic cycloalkyl group include a cyclopropyl group, a cyclobutyl group, a cyclopentyl group, a cyclohexyl group, a cycloheptyl group, a cyclooctyl group and a cyclodecyl group.

Examples of the polycyclic cycloalkyl group include a cycloalkyl group having a cross-linked structure, a cycloalkyl group in which two or more rings are condensed, and a cycloalkyl group in which two rings are bonded by spiro. Examples of the cycloalkyl group having a cross-linked structure include a norbornyl group and an adamantyl group. Examples of the cycloalkyl group in which two or more rings are condensed include a bicyclo[4,4,0]decane group and a steroid group (steroid skeleton). Examples of the cycloalkyl group in which two rings are bonded by spiro include a spirocyclic cycloalkyl group in which one type of cycloalkyl group selected from the group consisting of a cyclopentyl group, a cyclohexyl group, a norbornyl group and an adamantyl group, and a cycloalkyl group having 5 to 8 carbon atoms are bonded by spiro. A double bond may be formed between two carbon atoms contained in the alicyclic hydrocarbon group.

More specifically, alicyclic hydrocarbon groups in which a double bond is formed between two adjacent carbon atoms contained in the alicyclic hydrocarbon groups alicyclic hydrocarbon groups represented by the following formulas can be mentioned.

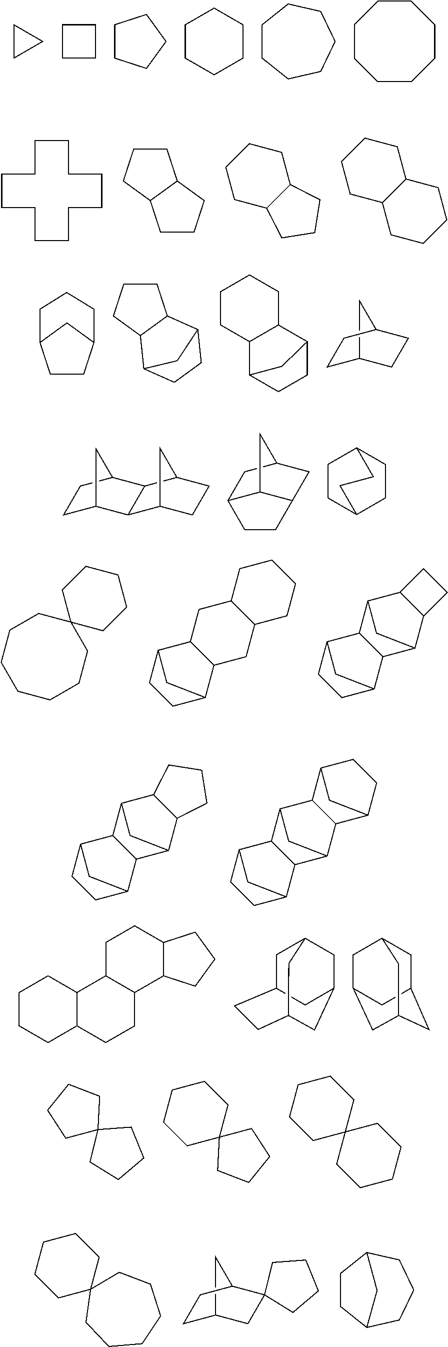

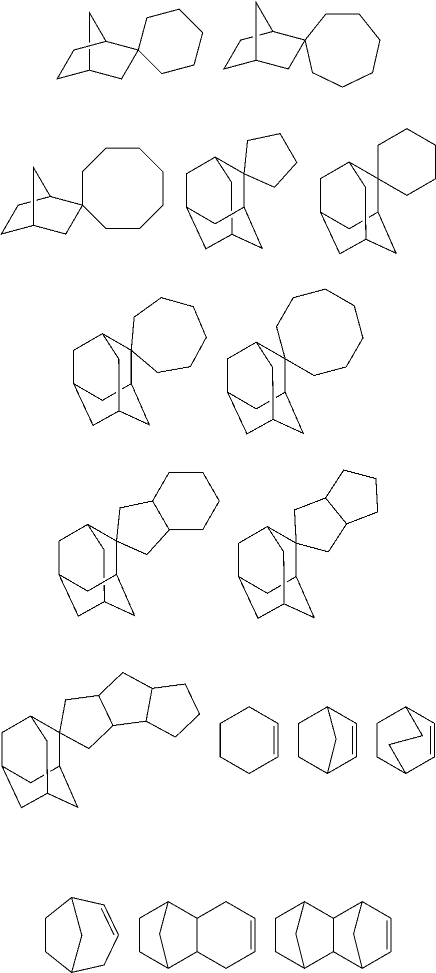

Furthermore, examples thereof also include a group in which one or more hydrogen atoms of the above groups are replaced with bonding sites.

In a case where the alicyclic hydrocarbon group is a monocyclic alicyclic hydrocarbon group, the number of carbon atoms of the alicyclic hydrocarbon group is preferably 3 to 24, is more preferably 3 to 20, is further preferably 3 to 18, is still more preferably 3 to 12, is still further preferably 3 to 10, and is further more preferably 3 to 8. In a case where the alicyclic hydrocarbon group is a polycyclic alicyclic hydrocarbon group, the number of carbon atoms of the alicyclic hydrocarbon group is preferably 6 to 24, is more preferably 6 to 20, is further preferably 6 to 18, is still more preferably 6 to 12, and is still further preferably 7 to 12.

Examples of the aromatic hydrocarbon group include an aryl group such as a phenyl group, a naphthyl group, an anthryl group, a biphenyl group and a fluorenyl group. The number of carbon atoms of the aromatic hydrocarbon group is preferably 4 to 24, is more preferably 4 to 20, is further preferably 4 to 18, is still more preferably 5 to 14, is still further preferably 5 to 10, and is further more preferably 6 to 10.

—CH₂— contained in the alicyclic hydrocarbon group, the aromatic hydrocarbon group, or the combined group of these groups is optionally replaced with —O—, —S—, —SO₂—, —SO—, —NR⁷— or —CO—, and examples of such groups formed by the replacement include groups similar to those described above as long as permitted by the upper limit of the number of carbon atoms.

The cyclic hydrocarbon group may be a cyclic hydrocarbon group having 3 to 26 carbon atoms, where the cyclic hydrocarbon group has an iodine atom and optionally has a substituent other than the iodine atom, and —CH₂-contained in the cyclic hydrocarbon group is optionally replaced with —O—, —S—, —SO₂—, —SO—, —NR⁷— or —CO—, is preferably a cyclic hydrocarbon group having 3 to 18 carbon atoms, where the cyclic hydrocarbon group has an iodine atom and optionally has a substituent other than the iodine atom, and —CH₂— contained in the cyclic hydrocarbon group is optionally replaced with —O—, —S—, —SO₂—, —SO—, —NR⁷— or —CO—, is more preferably an alicyclic hydrocarbon group having 3 to 18 carbon atoms or an aromatic hydrocarbon group having 6 to 18 carbon atoms, where the alicyclic hydrocarbon group and the aromatic hydrocarbon group have an iodine atom and optionally have a substituent other than the iodine atom, and —CH₂— contained in the alicyclic hydrocarbon group is optionally replaced with —O—, —S—, —SO₂—, —SO—, —NR⁷— or —CO—, and is further preferably a group represented by any one of the above formulas (w1-1) to (w1-33), provided that, in W², the group has an iodine atom.

Examples of the substituent other than the iodine atom that W² optionally has include the same groups as the substituents that W¹ optionally has. Specific examples thereof include a halogen atom other than the iodine atom, a cyano group, a nitro group, a haloalkyl group having 1 to 6 carbon atoms, and an alkyl group having 1 to 12 carbon atoms, where —CH₂— contained in the alkyl group is optionally replaced with —O—, —S—, —SO₂—, —SO—, —NR⁷— or —CO—.

Examples of the halogen atom other than the iodine atom include a fluorine atom, a chlorine atom and a bromine atom.

The number of iodine atoms that W² has is not particularly limited, but it is preferably 1 to 4, and is more preferably 1 or 2.

The number of substituents other than the iodine atom that W² optionally has is not particularly limited, but it is preferably 1 or 2.

In particular, W² may be a cyclic hydrocarbon group having 3 to 26 carbon atoms and an iodine atom, where —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom, is preferably a cyclic hydrocarbon group having 3 to 20 carbon atoms and an iodine atom, where —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom, is more preferably a cyclic hydrocarbon group having 3 to 18 carbon atoms and an iodine atom, where —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom, is further preferably a cyclic hydrocarbon group having 5 to 18 carbon atoms and an iodine atom, where the cyclic hydrocarbon group optionally has a substituent other than the iodine atom, is still more preferably an alicyclic hydrocarbon group having 5 to 12 carbon atoms and an iodine atom, where the alicyclic hydrocarbon group optionally has a substituent other than the iodine atom, or an aromatic hydrocarbon group having 6 to 10 carbon atoms and an iodine atom, where the aromatic hydrocarbon group optionally has a substituent other than the iodine atom, is still further preferably a cyclopentyl group having an iodine atom, where the cyclopentyl group optionally has a substituent other than the iodine atom, a cyclohexyl group having an iodine atom, where the cyclohexyl group optionally has a substituent other than the iodine atom, an adamantyl group having an iodine atom, where the adamantyl group optionally has a substituent other than the iodine atom, a phenyl group having an iodine atom, where the phenyl group optionally has a substituent other than the iodine atom, or a naphthyl group having an iodine atom, where the naphthyl group optionally has a substituent other than the iodine atom, is further more preferably an adamantyl group having an iodine atom, where the adamantyl group optionally has a substituent other than the iodine atom, or a phenyl group having an iodine atom, where the phenyl group optionally has a substituent other than the iodine atom, and still even more preferably an adamantyl group having an iodine atom, where the adamantyl group optionally has a substituent other than the iodine atom.

It is preferable that the bonding position of the iodine atom in W² is, for example, at a tertiary carbon atom of an adamantyl group, a secondary carbon atom of a cyclohexyl group and a cyclopentyl group, or the m-position with respect to the bonding position of A¹ of a phenyl group.

Examples of the anion (I) include the following anions. Among them, anions represented by formulas (Ia-1) to (Ia-12), (Ia-19), (Ia-22) to (Ia-50), and (Ia-101) to (Ia-120) are preferable.

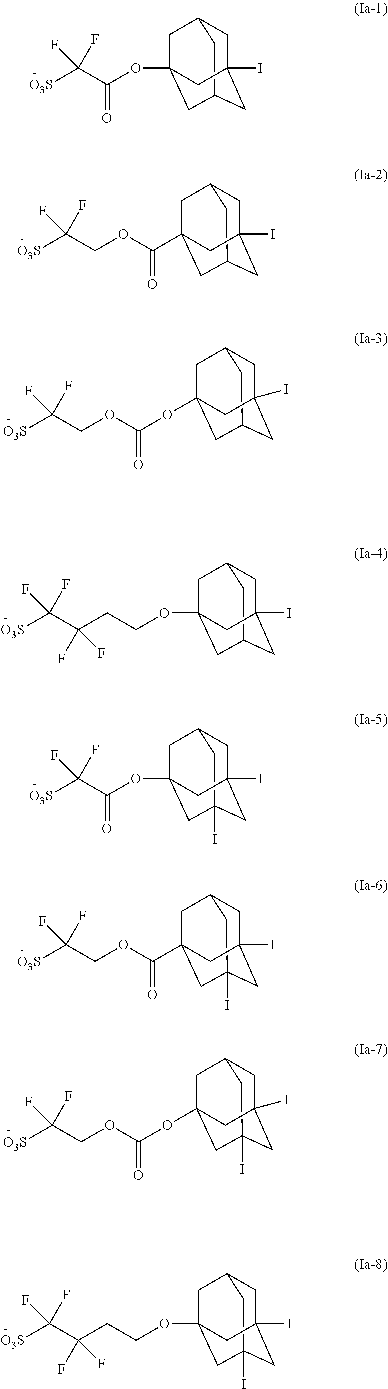

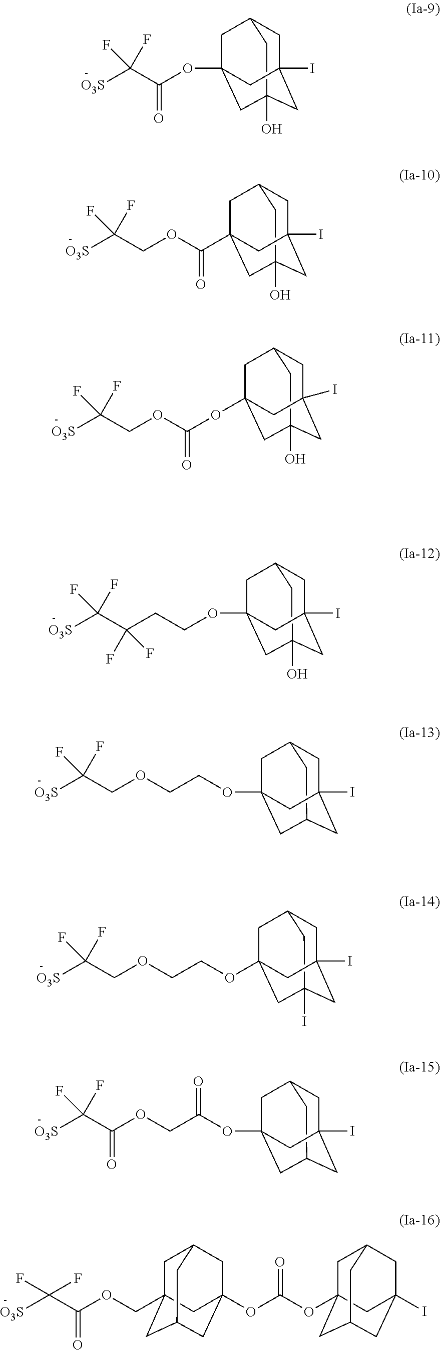

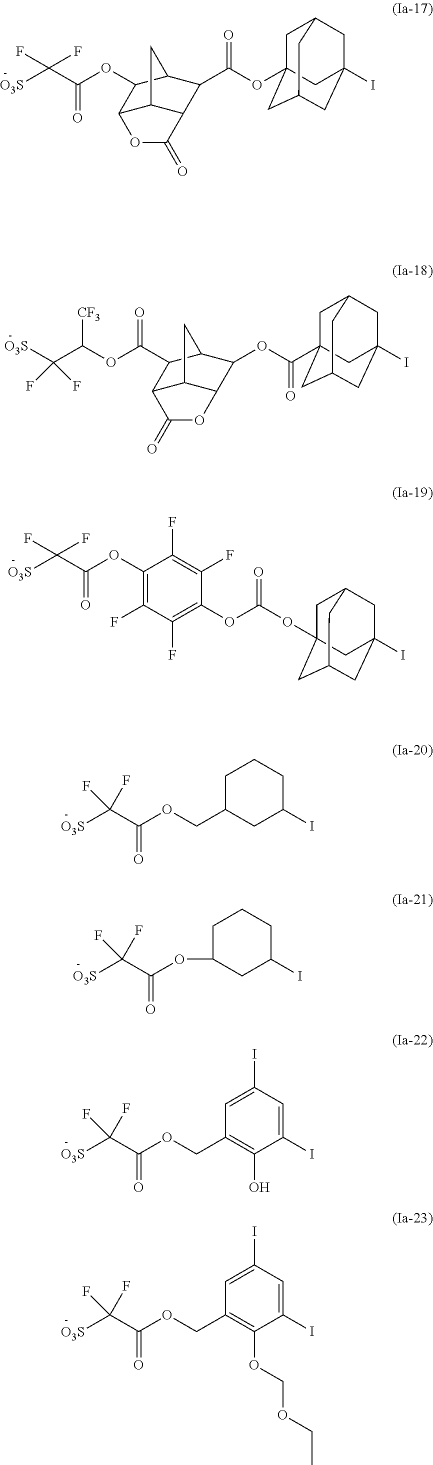

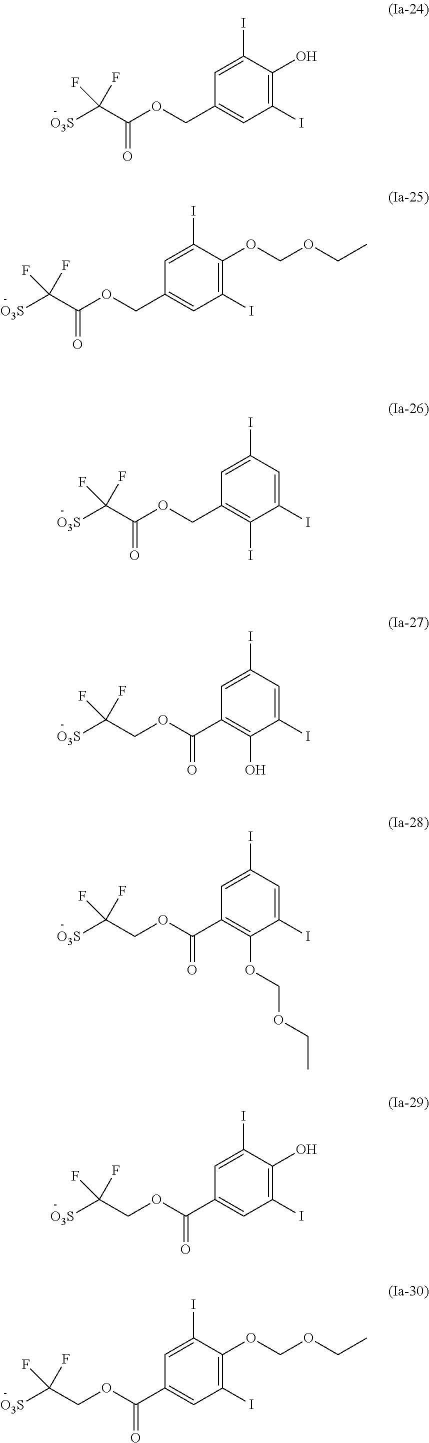

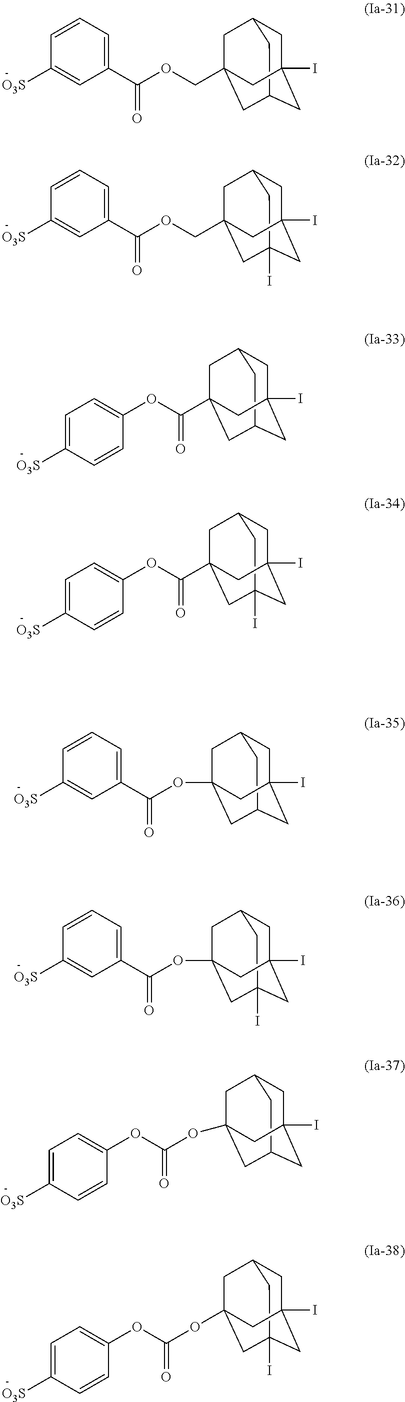

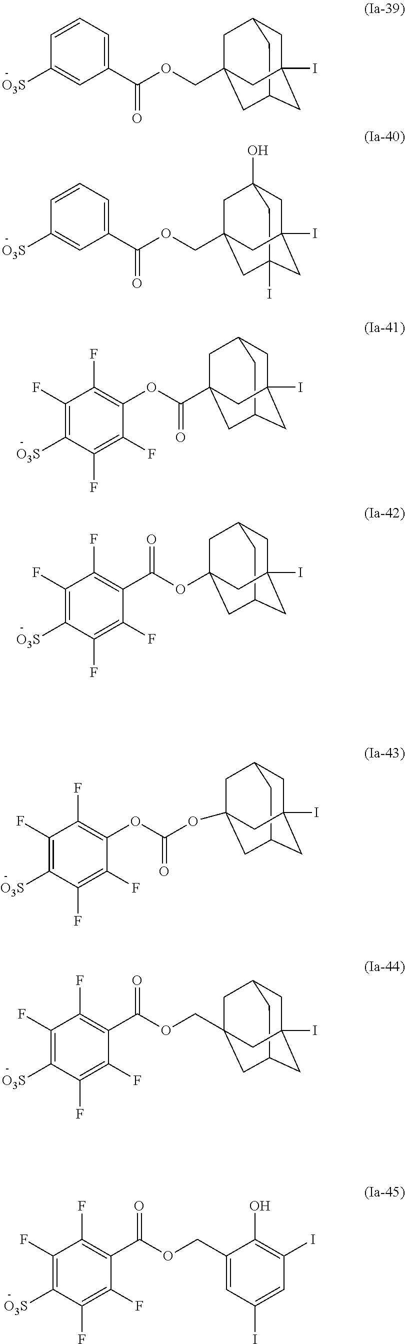

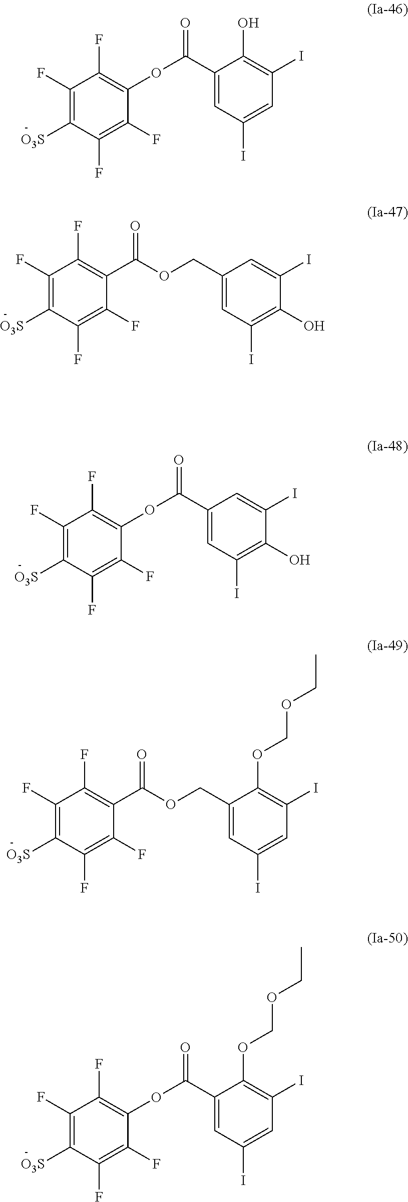

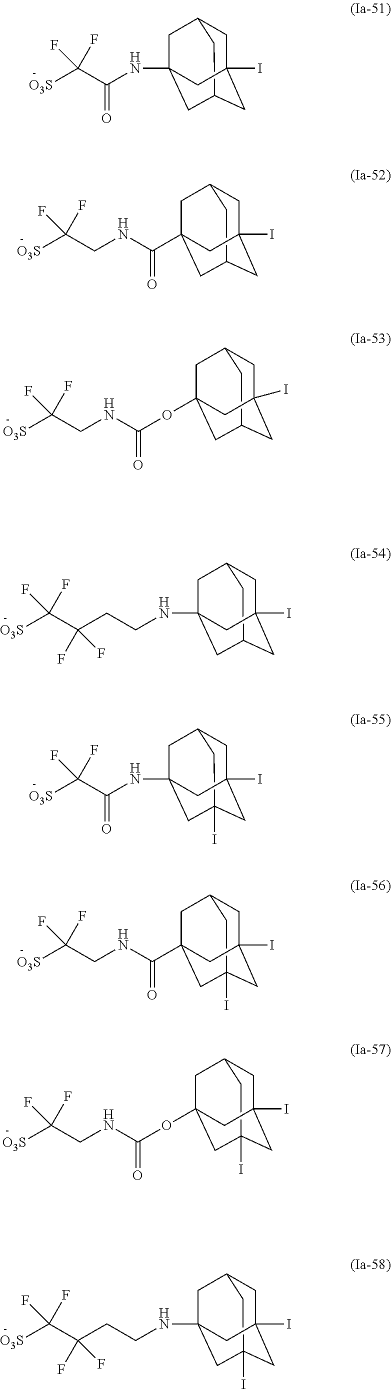

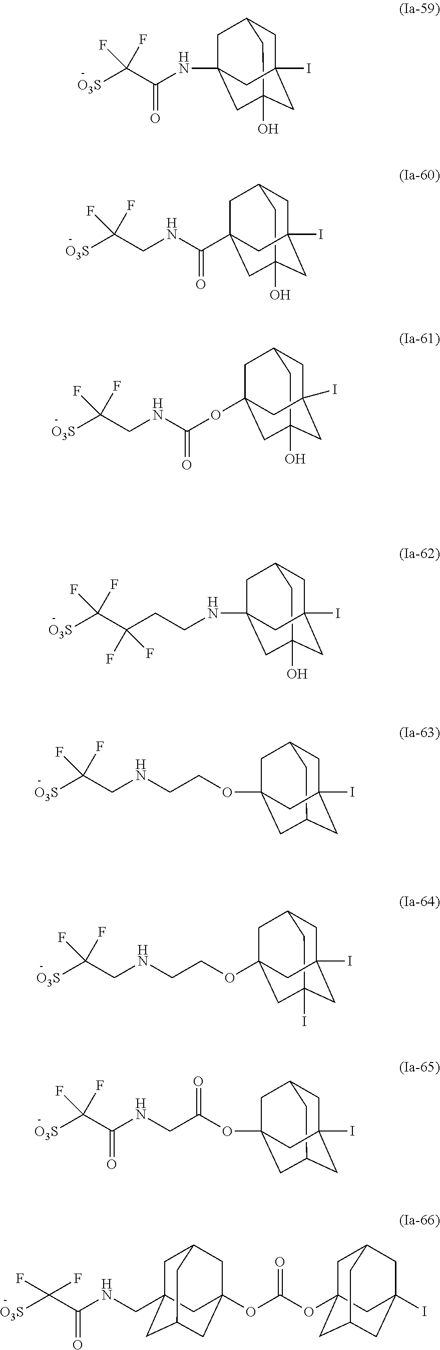

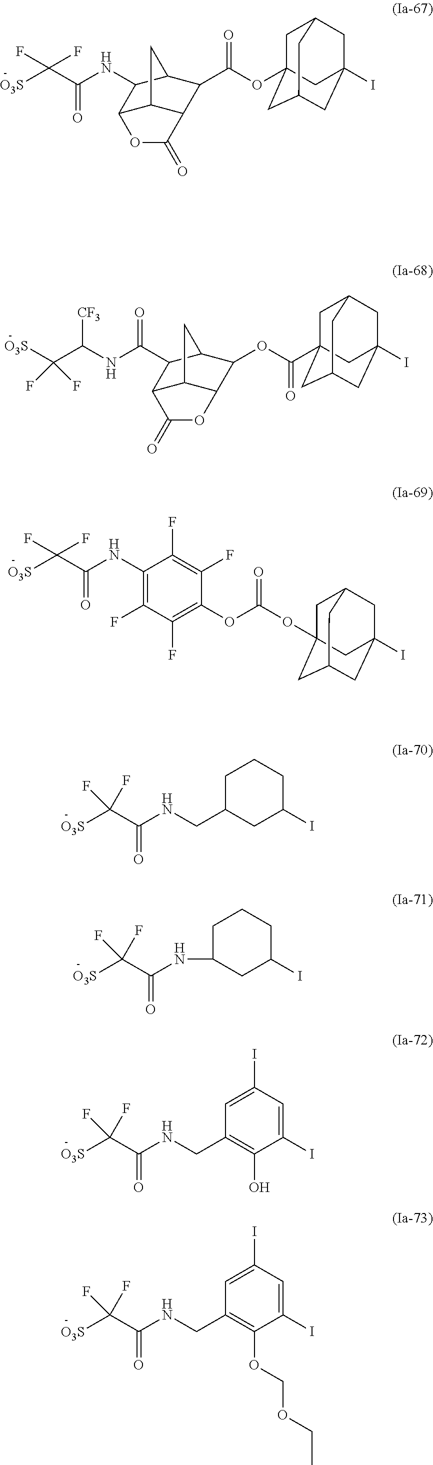

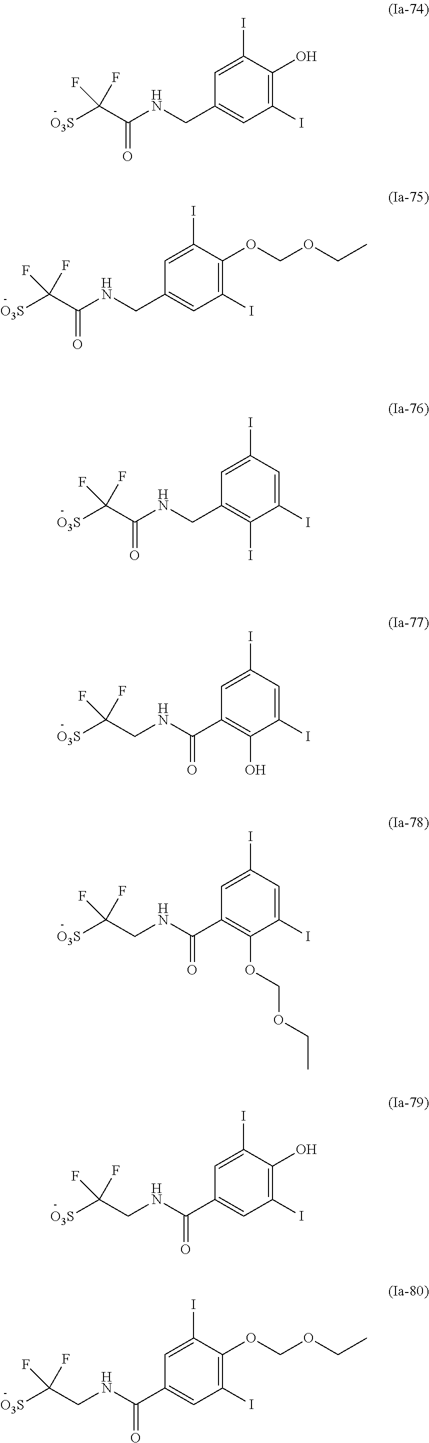

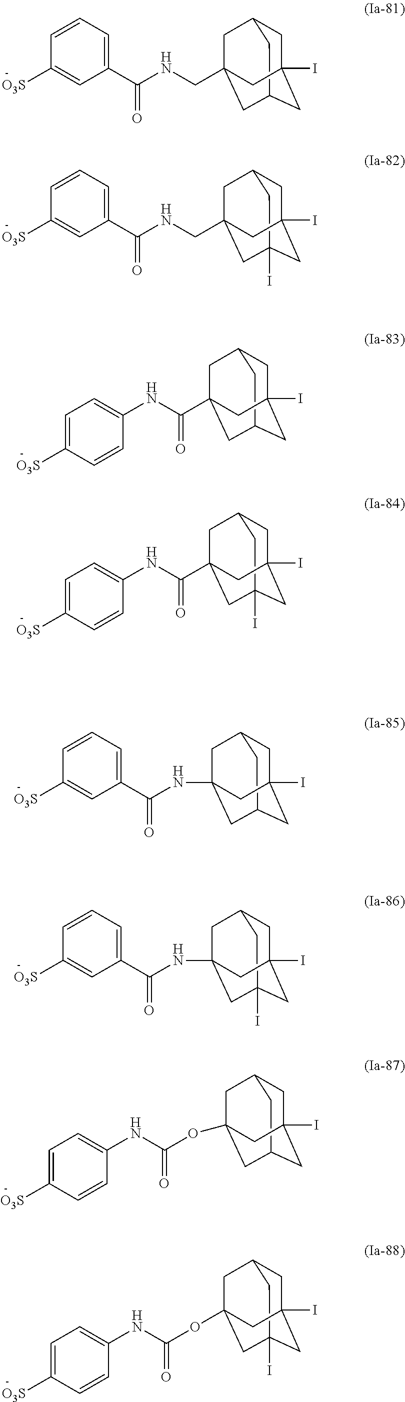

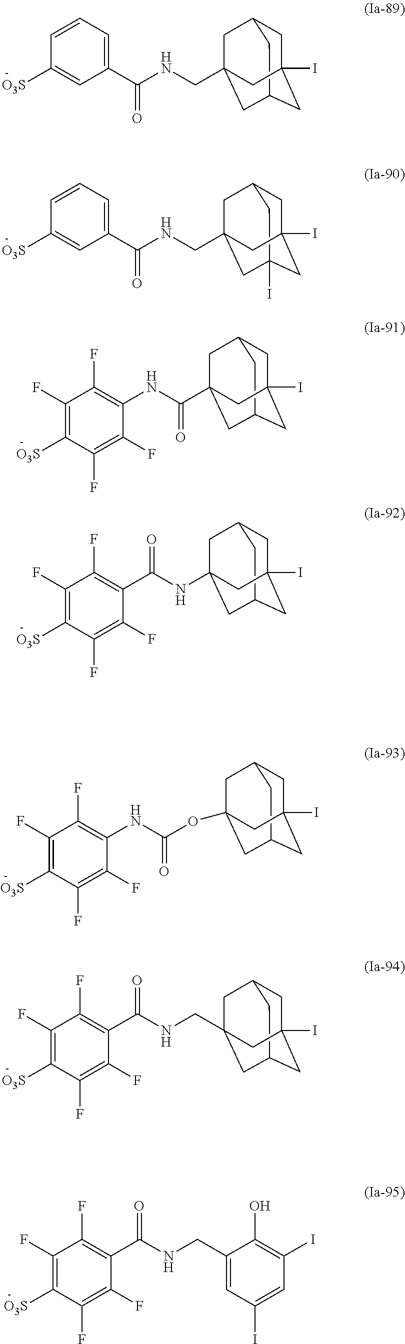

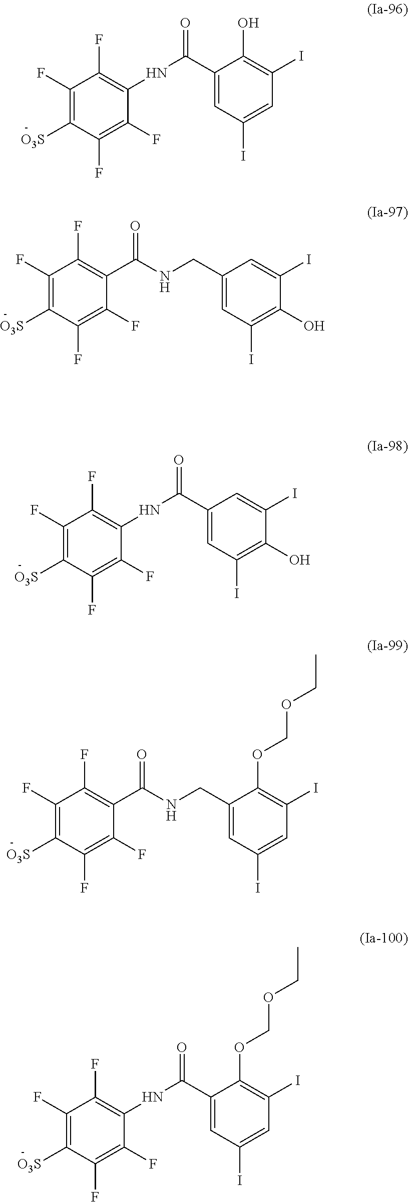

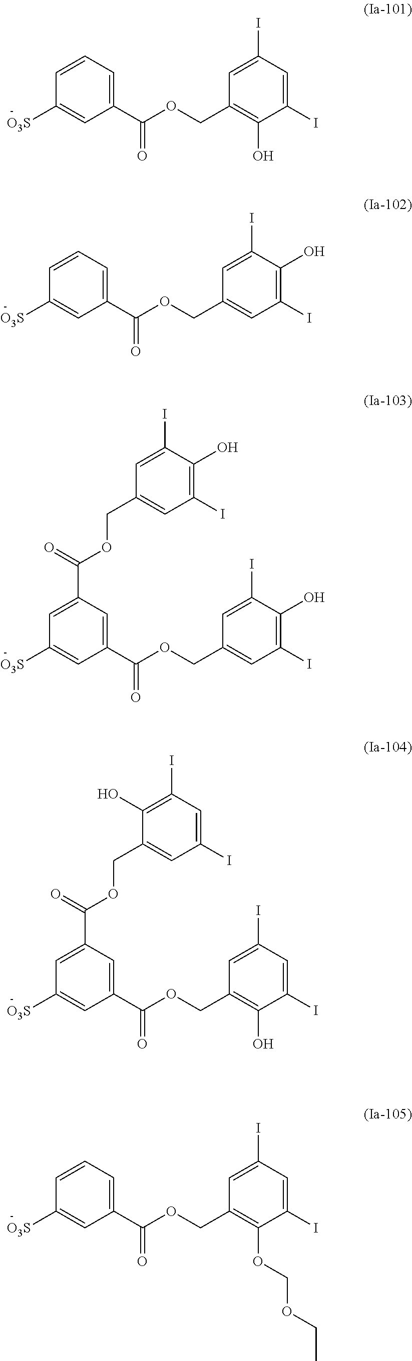

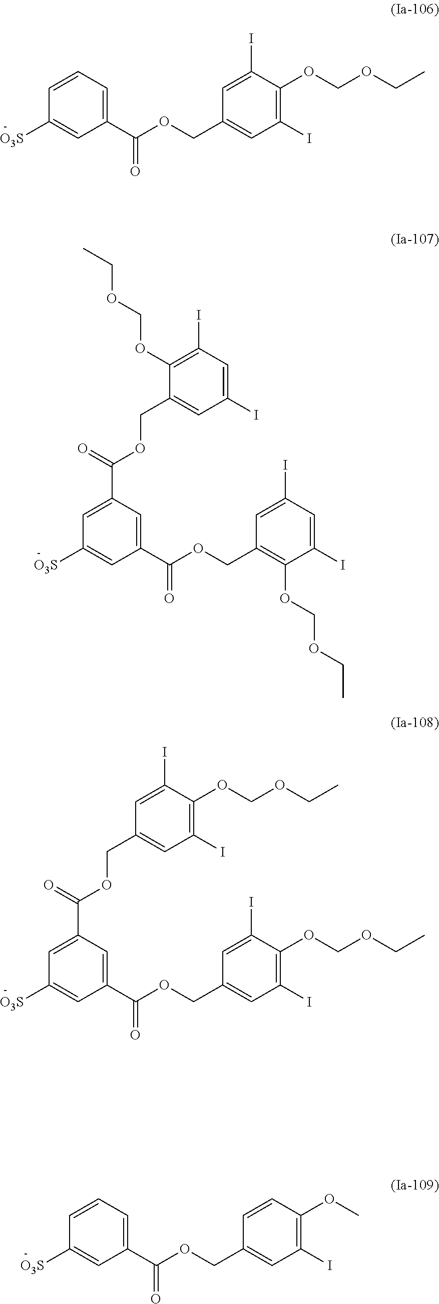

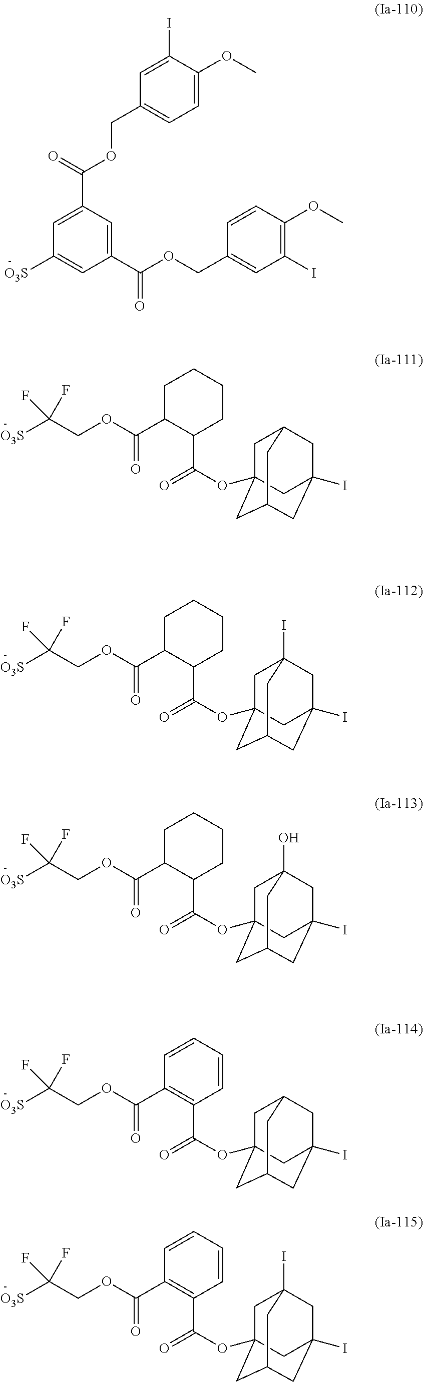

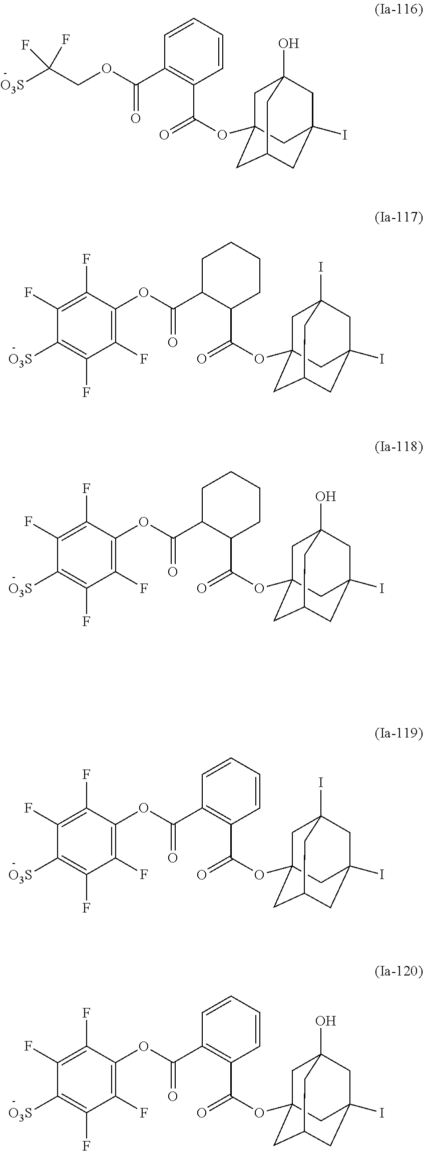

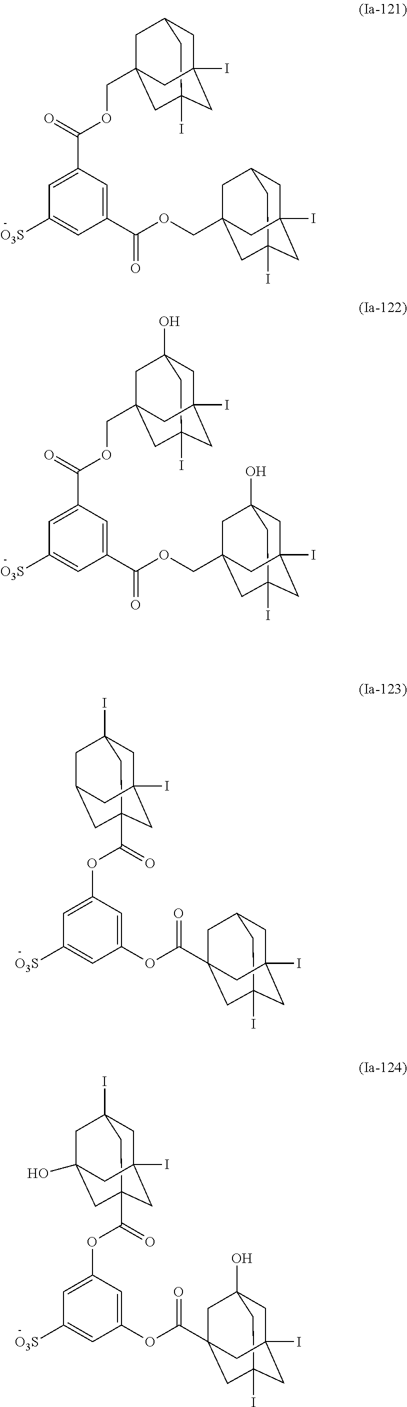

### [Cation (I)]

Examples of the halogen atom of R²⁰ include a fluorine atom, a chlorine atom, a bromine atom and an iodine atom.

Examples of the alkyl group of R²⁰ include a methyl group, an ethyl group, a n-propyl group, an isopropyl group, a n-butyl group, a sec-butyl group, a tert-butyl group, a n-pentyl group and a n-hexyl group, and it is preferably an alkyl group having 1 to 4 carbon atoms, and is more preferably a methyl group or an ethyl group.

Examples of the alkyl group having 1 to 6 carbon atoms and a halogen atom of R²⁰ include a fluorinated alkyl group having 1 to 6 carbon atoms, a chlorinated alkyl group having 1 to 6 carbon atoms, a brominated alkyl group having 1 to 6 carbon atoms, and an iodinated alkyl group having 1 to 6 carbon atoms. Specific examples of the alkyl group having a halogen atom include a trifluoromethyl group, a perfluoroethyl group, a perfluoropropyl group, a perfluoroisopropyl group, a perfluorobutyl group, a perfluoro-sec-butyl group, a perfluoro-tert-butyl group, a perfluoropentyl group, a perfluorohexyl group, a perchloromethyl group, a perbromomethyl group and a periodomethyl group.

The number of carbon atoms of the alkyl group, the fluorinated alkyl group, the chlorinated alkyl group, the brominated alkyl group, and the iodinated alkyl group is preferably 1 to 4, is more preferably 1 to 3, and is further preferably 1 or 2.

R²⁰ is preferably a hydrogen atom or an alkyl group having 1 to 4 carbon atoms, is more preferably a hydrogen atom or an alkyl group having 1 to 3 carbon atoms, is further preferably a hydrogen atom or a methyl group, and is further more preferably a methyl group.

X²⁰ is preferably a single bond, —O—, —CO—, —NR⁷—, a phenylene group optionally having a substituent, or a combined group of these groups, and is more preferably a single bond, *—O—**, *—CO—**, *—CO—O—**, *—O—CO—O—**, * CO—NR⁷—**, *—NR⁷—CO—O—**, *—O—CO—NR⁷—** or *-Ax-Ph-Ay-**.

In the above groups, * and ** represent bonding sites, and * represents a bonding site to a carbon atom to which R²⁰ is bonded. Ax and Ay each independently represent one or more types of bonds selected from the group consisting of a single bond, an ether bond, a thioether bond, an amide bond, an ester bond and a carbonate ester bond. Ph represents a phenylene group optionally having a substituent.

When X²⁰ is a group represented by *-Ax-Ph-Ay-**, it is preferably a linking group represented by the following formula (X20):

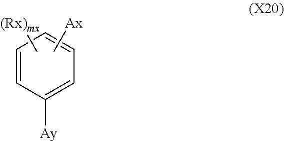

wherein
Ax and Ay have the same meanings as described above;
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms; and
mx represents an integer of 0 to 4, and when mx is an integer of 2 or larger, a plurality of Rx may be the same or different from each other.

When either Ax or Ay is a single bond, the other is preferably one or more selected from the group consisting of an ether bond, a thioether bond, an amide bond, an ester bond and a carbonate ester bond.

When Ax and Ay are amide bonds, it is preferable that they are —NR⁷—.

Rx is preferably a fluorine atom, an iodine atom, a trifluoromethyl group, a methyl group or an ethyl group.

mx is preferably 0, 1 or 2.

The bonding position of Ay in the phenylene group is preferably the m-position or p-position with respect to the bonding position of Ax, and is more preferably the p-position.

Examples of X²⁰ include the groups represented by the following formulas (X²⁰-1) to (X²⁰-10). * represents a bonding site to a carbon atom to which —R²⁰ is bonded. ** represents a bonding site to L²⁰:

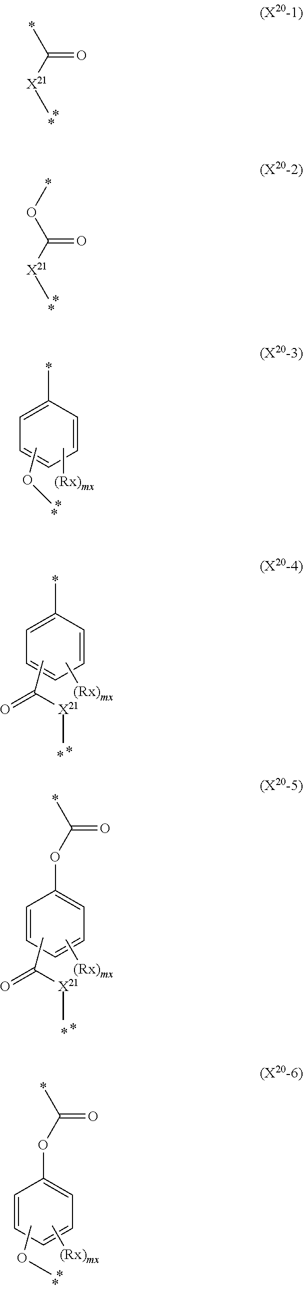

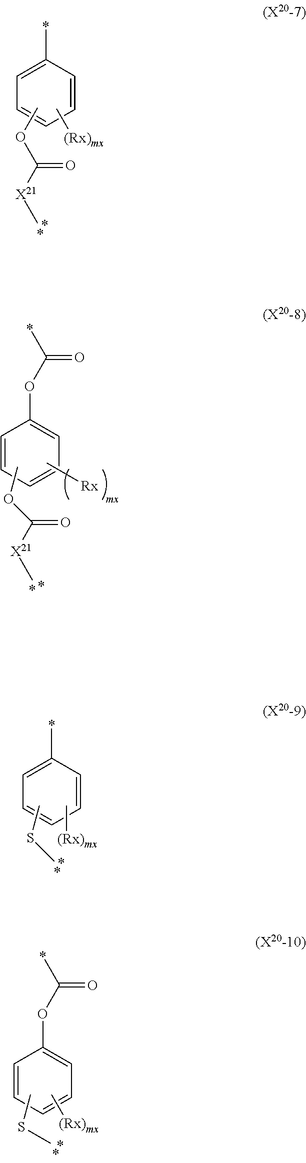

wherein all symbols in formulas (X²⁰-1) to (X²⁰-10) have the same meanings as described above.

Specific examples of the groups represented by formulas (X²⁰-1) to (X²⁰-10) include the following groups.

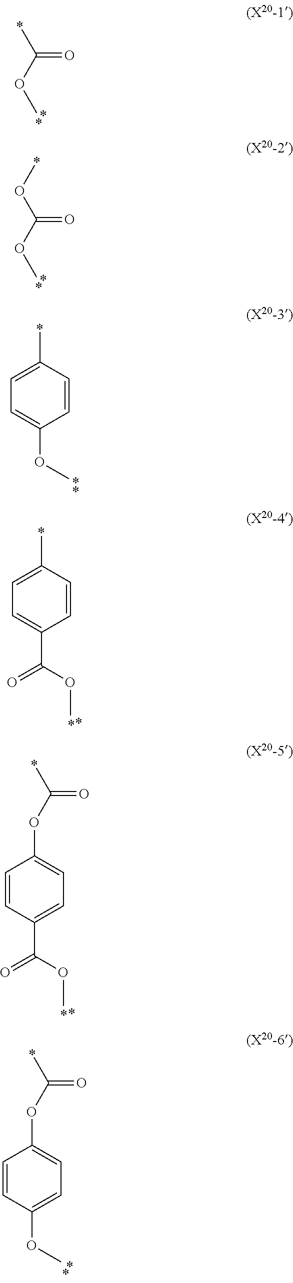

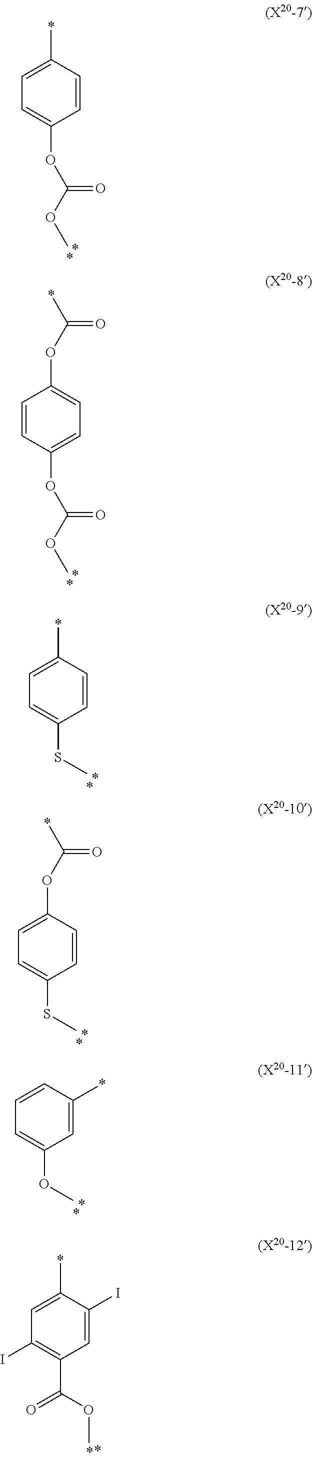

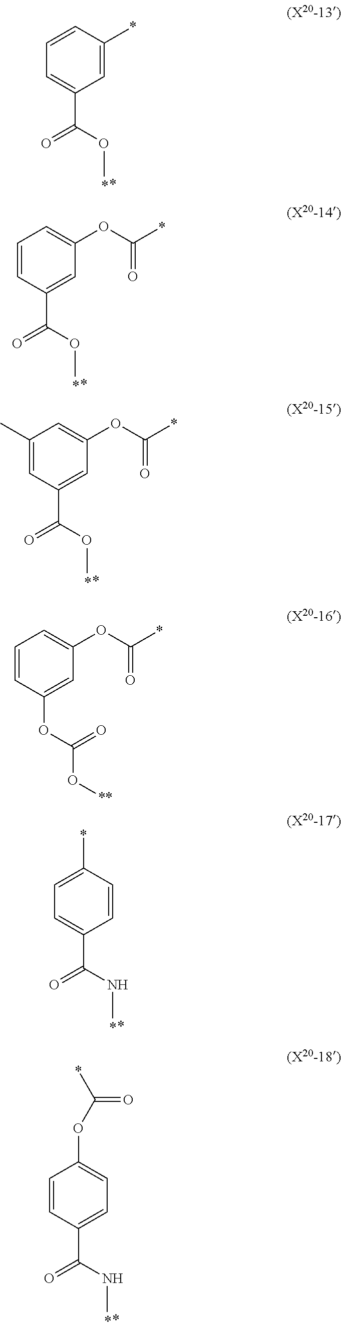

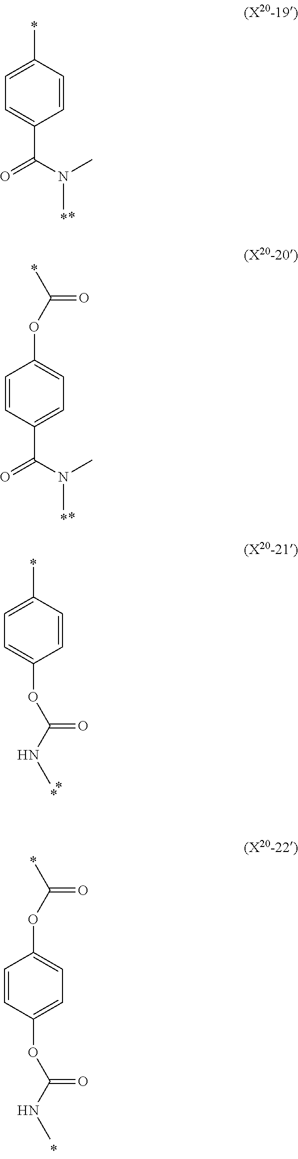 [SMILES: *c1ccc(C(=O)N(C)C)cc1 ; *C(=O)Oc1ccc(C(=O)N(C)C)cc1 ; *c1ccc(OC(=O)NC)cc1 ; *C(=O)Oc1ccc(OC(=O)NC)cc1]

In particular, X²⁰ is preferably a single bond or a group represented by any one of formulas (X²⁰-1′) and (X²⁰-3′) to (X²⁰-10′), is more preferably a single bond or a group represented by any one of formulas (X²⁰-1′), (X²⁰-4′), (X²⁰-5′), (X²⁰-6′) and (X²⁰-10′), and is further preferably a single bond, a group represented by formula (X²⁰-1′), a group represented by formula (X²⁰-5′) or a group represented by formula (X²⁰-6′).

Examples of the hydrocarbon group having 1 to 36 carbon atoms of L¹⁰ and L²⁰ include an aliphatic hydrocarbon group (a straight-chain or branched chain hydrocarbon group such as an alkanediyl group, an alkenediyl group and an alkynediyl group, and a monocyclic or polycyclic, saturated or unsaturated alicyclic hydrocarbon group), and an aromatic hydrocarbon group, and it may also be a combined hydrocarbon group of two or more of these groups. The number of carbon atoms of the hydrocarbon group is preferably 1 to 32, is more preferably 1 to 28, is further preferably 1 to 24, and is further more preferably 1 to 18.

Examples of the alkanediyl group include: a straight-chain alkanediyl group such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and

a branched alkanediyl group such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The terminal of the branched alkanediyl group may be an alkyl group such as a methyl group.

Examples of the alkenediyl group include an ethenediyl group, a propenediyl group, an isopropenediyl group, a butenediyl group, an isobutenediyl group, a tert-butenediyl group, a pentenediyl group, a hexenediyl group, a heptenediyl group, an octenediyl group, an isooctenediyl group and a nonenediyl group.

Examples of the alkynediyl group include an ethynediyl group, a propynediyl group, an isopropynediyl group, a butynediyl group, an isobutynediyl group, a tert-butynediyl group, a pentynediyl group, a hexynediyl group, an octynediyl group and a nonynediyl group.

The number of carbon atoms of the chain hydrocarbon group may be 1 to 18, is preferably 1 to 12, is more preferably 1 to 10, is further preferably 1 to 9, is further more preferably 1 to 8, is even more preferably 1 to 6, is still more preferably 1 to 4, and is still further preferably 1 to 3.

The alicyclic hydrocarbon group may be any of monocyclic, polycyclic and spiro rings. Examples of the divalent alicyclic hydrocarbon group include the groups represented as follows. The bonding site can be set to any position.

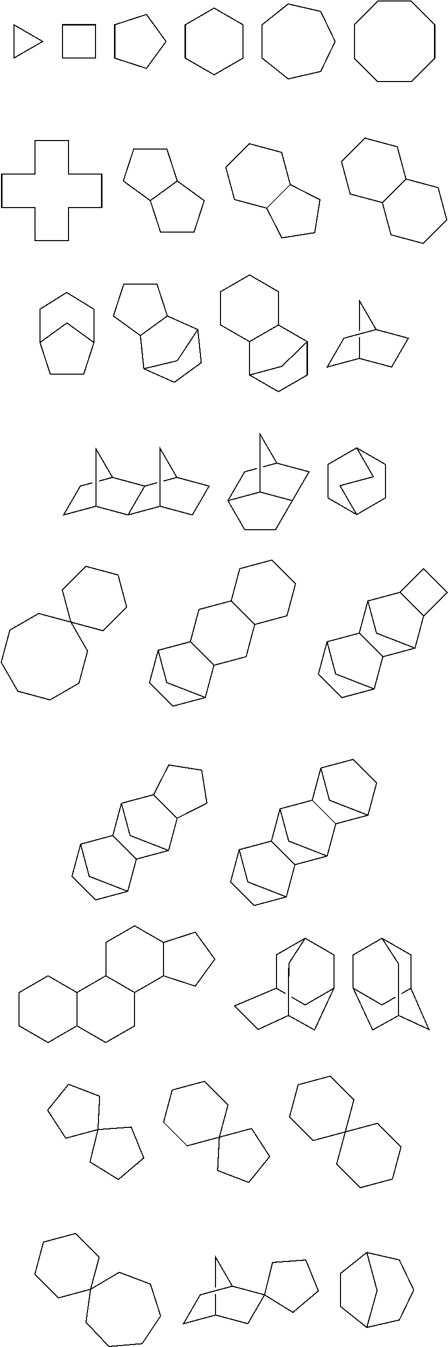

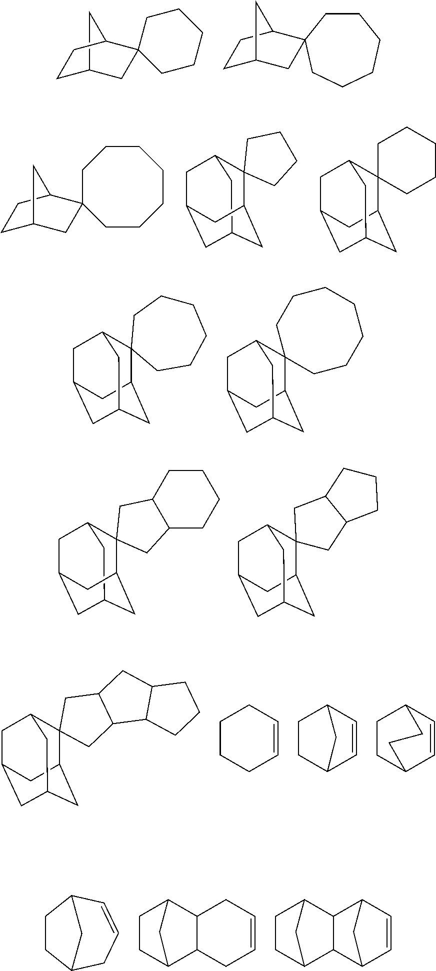

Specifically, examples of the monocyclic divalent alicyclic hydrocarbon group include a monocyclic cycloalkanediyl group such as a cyclobutane-1,3-diyl group, a cyclopentane-1,3-diyl group, a cyclohexane-1,4-diyl group, a cyclohexene-3,6-diyl group and a cyclooctane-1,5-diyl group, and examples of the polycyclic divalent alicyclic hydrocarbon group include a polycyclic cycloalkanediyl group such as a norbornane-1,4-diyl group, a norbornane-2,5-diyl group, a 5-norbornene-2,3-diyl group, an adamantane-1,5-diyl group and an adamantane-2,6-diyl group.

The number of carbon atoms of the alicyclic hydrocarbon group may be 3 to 24, is preferably 3 to 18, is more preferably 3 to 16, and is further preferably 3 to 12.

Examples of the aromatic hydrocarbon group include a phenylene group, a naphthylene group, an anthrylene group, a biphenylene group and a phenanthrylene group. The number of carbon atoms of the aromatic hydrocarbon group may be 6 to 24, is preferably 6 to 18, is more preferably 6 to 14, and is further preferably 6 to 10.

Examples of the combined hydrocarbon group of two or more include a combined group of an alkanediyl group and an alicyclic hydrocarbon group and/or an aromatic hydrocarbon group, such as -alicyclic hydrocarbon group-alkanediyl group-, -alkanediyl group-alicyclic hydrocarbon group-, -alkanediyl group-alicyclic hydrocarbon group-alkanediyl group-, -alkanediyl group-aromatic hydrocarbon group- and -aromatic hydrocarbon group-alkanediyl group-.

—CH₂— contained in the hydrocarbon group having 1 to 36 carbon atoms of L¹⁰ and L²⁰ is optionally replaced with —O—, —S—, —CO—, —SO—, —NR⁷— or —SO₂—.

When the hydrocarbon group having 1 to 36 carbon atoms of L¹⁰ and L²⁰ has a substituent or when —CH₂— contained in the hydrocarbon group is replaced with —O—, —S—, —CO—, —SO—, —NR⁷— or —SO₂—, the number of carbon atoms before replacement corresponds to the defined number of carbon atoms of the hydrocarbon group.

Examples of the group formed by replacing —CH₂— contained in the hydrocarbon group with —O—, —S—, —SO₂—, —SO—, —NR⁷— or —CO— include a hydroxy group, a carboxy group, a thiol group, an amino group, a carbonyl group, a sulfonyl group, an oxy group, a thio group, a peptide group, an alkoxy group, an alkoxycarbonyl group, an alkylcarbonyl group, an alkylcarbonyloxy group, an alkoxycarbonyloxy group, an alkoxycarbonyloxy group, an alkoxyalkoxy group, an alkoxyalkyl group, a hydroxyalkyl group, an alkanediyloxy group, an alkanediyloxycarbonyl group, an alkanediylcarbonyl group, an alkanediylcarbonyloxy group, an alkanediyloxycarbonyloxy group, an alkylthio group, an alkylsulfonyl group, an alkanediylsulfonyl group, an alkanediylthio group, an alkylamino group, an alkanediylamino group, an alkyl peptide group, an alkanediyl peptide group, a cycloalkoxy group, a cycloalkylalkoxy group, an aromatic hydrocarbon group -carbonyloxy group, an aromatic hydrocarbon group-carbonyl group, an aromatic hydrocarbon group-oxy group and a combined group of two or more of these groups. Examples of these groups formed by the replacement include the same groups as those exemplified in the present specification, within the range permitted by the upper limit of the number of carbon atoms.

In addition, examples of the group formed by replacing —CH₂-contained in the alicyclic hydrocarbon group, the aromatic hydrocarbon group or the combined group of an alicyclic hydrocarbon group and an aromatic hydrocarbon group with —O—, —S—, —CO—, —SO—, —NR⁷— or —SO₂— include the same groups as those exemplified in the present specification, within the range permitted by the upper limit of the number of carbon atoms.

Examples of the substituent that the hydrocarbon group of L¹⁰ and L²⁰ optionally has include a halogen atom, a cyano group and a nitro group.

When L¹⁰ and L²⁰ are combined groups of an alicyclic hydrocarbon group or an aromatic hydrocarbon group and a chain hydrocarbon group such as alkyl group, the chain hydrocarbon group such as alkyl group can substantially be a substituent for the alicyclic hydrocarbon group or the aromatic hydrocarbon group. In addition, when —CH₂— contained in the chain hydrocarbon group included in L¹⁰ and L²⁰, such as alkyl group, is replaced with —O—, —S—, —SO₂—, SO—, —NR⁷— or —CO—, L¹⁰ and L²⁰ can substantially have a substituent such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group and an alkyl peptide group.

Examples of the halogen atom include a fluorine atom, a chlorine atom, a bromine atom and an iodine atom.

The hydrocarbon group having 1 to 36 carbon atoms of L¹⁰ and L²⁰ optionally has one substituent or a plurality of substituents.

Examples of L¹⁰ include a single bond, a chain hydrocarbon group having 1 to 18 carbon atoms and optionally having a substituent, where —CH₂-contained in the chain hydrocarbon group is optionally replaced with —O—, —S—, —SO₂—, —SO—, —NR⁷— or —CO—, a cyclic hydrocarbon group having 3 to 24 carbon atoms and optionally having a substituent, where —CH₂— contained in the cyclic hydrocarbon group is optionally replaced with —O—, —S—, —SO₂—, —SO—, —NR⁷— or —CO—, and a combined group of a chain hydrocarbon group having 1 to 18 carbon atoms and optionally having a substituent and a cyclic hydrocarbon group having 3 to 24 carbon atoms and optionally having a substituent, where —CH₂-contained in the chain hydrocarbon group and the cyclic hydrocarbon group is optionally replaced with —O—, —S—, —SO₂—, —SO—, —NR⁷— or —CO—, and it may be a single bond, a chain hydrocarbon group having 1 to 8 carbon atoms and optionally having a substituent, where —CH₂— contained in the chain hydrocarbon group is optionally replaced with —O—, —S—, —SO₂—, —SO—, —NR⁷— or —CO—, a cyclic hydrocarbon group having 3 to 18 carbon atoms and optionally having a substituent, where —CH₂— contained in the cyclic hydrocarbon group is optionally replaced with —O—, —S—, —SO₂—, —SO—, —NR⁷— or —CO—, or a combined group of a chain hydrocarbon group having 1 to 8 carbon atoms and optionally having a substituent and a cyclic hydrocarbon group having 3 to 18 carbon atoms and optionally having a substituent, where —CH₂— contained in the chain hydrocarbon group and the cyclic hydrocarbon group is optionally replaced with —O—, —S—, —SO₂—, —SO—, —NR⁷— or —CO—, is preferably a single bond, an alkanediyl group having 1 to 8 carbon atoms and optionally having a substituent, where —CH₂-contained in the alkanediyl group is optionally replaced with —O—, —S—, —SO₂— or —CO—, a cyclic hydrocarbon group having 3 to 18 carbon atoms and optionally having a substituent, where —CH₂— contained in the cyclic hydrocarbon group is optionally replaced with —O—, —S—, —SO₂— or —CO—, or a combined group of an alkanediyl group having 1 to 8 carbon atoms and optionally having a substituent and a cyclic hydrocarbon group having 3 to 18 carbon atoms and optionally having a substituent, where —CH₂— contained in the alkanediyl group and the cyclic hydrocarbon group is optionally replaced with —O—, —S—, —SO₂— or —CO—, is more preferably a single bond, or a combined group of an alkanediyl group having 1 to 6 carbon atoms, where —CH₂— contained in the alkanediyl group is optionally replaced with —O—, —S—, —SO₂— or —CO—, and an aromatic hydrocarbon group having 6 to 18 carbon atoms and optionally having a substituent, and is further preferably a single bond, or a *—O-phenylene group or a *—S-phenylene group, where the phenylene group optionally has a substituent and * represents a bonding site to a benzene ring.

Examples of L²⁰ include a single bond, a chain hydrocarbon group having 1 to 18 carbon atoms, where —CH₂— contained in the chain hydrocarbon group is optionally replaced with —O—, —S—, —SO₂—, —SO—, —NR⁷— or —CO—, a cyclic hydrocarbon group having 3 to 24 carbon atoms, where the cyclic hydrocarbon group optionally has a substituent and —CH₂— contained in the cyclic hydrocarbon group is optionally replaced with —O—, —S—, —SO₂—, —SO—, —NR⁷— or —CO—, and a group formed by combining a chain hydrocarbon group having 1 to 18 carbon atoms and a cyclic hydrocarbon group having 3 to 24 carbon atoms, where the cyclic hydrocarbon group optionally has a substituent, —CH₂— contained in the chain hydrocarbon group is optionally replaced with —O—, —S—, —SO₂—, —SO—, —NR⁷— or —CO—, and —CH₂— contained in the cyclic hydrocarbon group is optionally replaced with —O—, —S—, —SO₂—, —SO—, —NR⁷— or —CO—, and it may be a single bond, a chain hydrocarbon group having 1 to 12 carbon atoms, where —CH₂— contained in the chain hydrocarbon group is optionally replaced with —O—, —S—, —SO₂—, —SO—, —NR⁷— or —CO—, a cyclic hydrocarbon group having 3 to 18 carbon atoms, where the cyclic hydrocarbon group optionally has a substituent and —CH₂— contained in the cyclic hydrocarbon group is optionally replaced with —O—, —S—, —SO₂—, —SO—, —NR⁷— or —CO—, or a group formed by combining a chain hydrocarbon group having 1 to 12 carbon atoms and a cyclic hydrocarbon group having 3 to 18 carbon atoms, where the cyclic hydrocarbon group optionally has a substituent, —CH₂— contained in the chain hydrocarbon group is optionally replaced with —O—, —S—, —SO₂—, —SO—, —NR⁷— or —CO—, and —CH₂— contained in the cyclic hydrocarbon group is optionally replaced with —O—, —S—, —SO₂—, —SO—, —NR⁷— or —CO—, is preferably a single bond, an alkanediyl group having 1 to 12 carbon atoms, where —CH₂— contained in the alkanediyl group is optionally replaced with —O— or —CO—, a cyclic hydrocarbon group having 3 to 18 carbon atoms, where the cyclic hydrocarbon group optionally has a substituent and —CH₂— contained in the cyclic hydrocarbon group is optionally replaced with —O—, —S—, —SO₂— or —CO—, or a group formed by combining an alkanediyl group having 1 to 12 carbon atoms and a cyclic hydrocarbon group having 3 to 18 carbon atoms, where the cyclic hydrocarbon group optionally has a substituent, —CH₂— contained in the alkanediyl group is optionally replaced with —O— or —CO—, and —CH₂— contained in the cyclic hydrocarbon group is optionally replaced with —O—, —S—, —SO₂— or —CO—, is more preferably a single bond, an alkanediyl group having 1 to 6 carbon atoms, where —CH₂— contained in the alkanediyl group is optionally replaced with —O— or —CO—, an alicyclic hydrocarbon group having 3 to 18 carbon atoms, where —CH₂— contained in the alicyclic hydrocarbon group is optionally replaced with —O—, —S—, —SO₂— or —CO—, an aromatic hydrocarbon group having 6 to 18 carbon atoms, a group formed by combining an alkanediyl group having 1 to 6 carbon atoms and an alicyclic hydrocarbon group having 3 to 18 carbon atoms, where —CH₂— contained in the alkanediyl group is optionally replaced with —O— or —CO—, and —CH₂— contained in the alicyclic hydrocarbon group is optionally replaced with —O—, —S—, —SO₂— or —CO—, or a group formed by combining an alkanediyl group having 1 to 6 carbon atoms and an aromatic hydrocarbon group having 6 to 18 carbon atoms, where —CH₂— contained in the alkanediyl group is optionally replaced with —O— or —CO—, is further preferably a single bond, an alkanediyl group having 1 to 6 carbon atoms, where —CH₂— contained in the alkanediyl group is optionally replaced with —O— or —CO—, an alicyclic hydrocarbon group having 3 to 18 carbon atoms, where —CH₂— contained in the alicyclic hydrocarbon group is optionally replaced with —O— or —CO—, a phenylene group optionally having a substituent, a group formed by combining an alkanediyl group having 1 to 6 carbon atoms and an alicyclic hydrocarbon group having 3 to 18 carbon atoms, where —CH₂— contained in the alkanediyl group is optionally replaced with —O— or —CO—, and —CH₂— contained in the alicyclic hydrocarbon group is optionally replaced with —O—or —CO—, or a group formed by combining an alkanediyl group having 1 to 6 carbon atoms and a phenylene group optionally having a substituent, where —CH₂— contained in the alkanediyl group is optionally replaced with —O— or —CO—, and is further more preferably a single bond or an alkanediyl group having 1 to 4 carbon atoms, where —CH₂— contained in the alkanediyl group is optionally replaced with —O— or —CO—.

The bonding position of L¹⁰ to a benzene ring may be at any of the o-position, m-position and p-position, with respect to the bonding position of L²⁰. In particular, L¹⁰ is preferably bonded to the p-position or m-position with respect to the bonding position of L²⁰, and is more preferably bonded to the p-position.

Examples of the hydrocarbon group having 1 to 36 carbon atoms of R² include a straight-chain or branched chain hydrocarbon group such as an alkyl group, a monocyclic or polycyclic alicyclic hydrocarbon group, an aromatic hydrocarbon group, and a combined group of these groups.

Examples of the alkyl group include an alkyl group such as a methyl group, an ethyl group, a propyl group, an isopropyl group, a butyl group, an isobutyl group, a tert-butyl group, a pentyl group, a hexyl group, an octyl group and a nonyl group. The number of carbon atoms of the chain hydrocarbon group may be 1 to 18, is preferably 1 to 12, is more preferably 1 to 10, is further preferably 1 to 9, is further more preferably 1 to 8, is even more preferably 1 to 6, is still more preferably 1 to 4, and is still further preferably 1 to 3.

The alicyclic hydrocarbon group may be any of monocyclic, polycyclic and spiro rings, and may be either saturated or unsaturated. Examples of the alicyclic hydrocarbon group include the groups represented as follows. The bonding site can be set to any position.

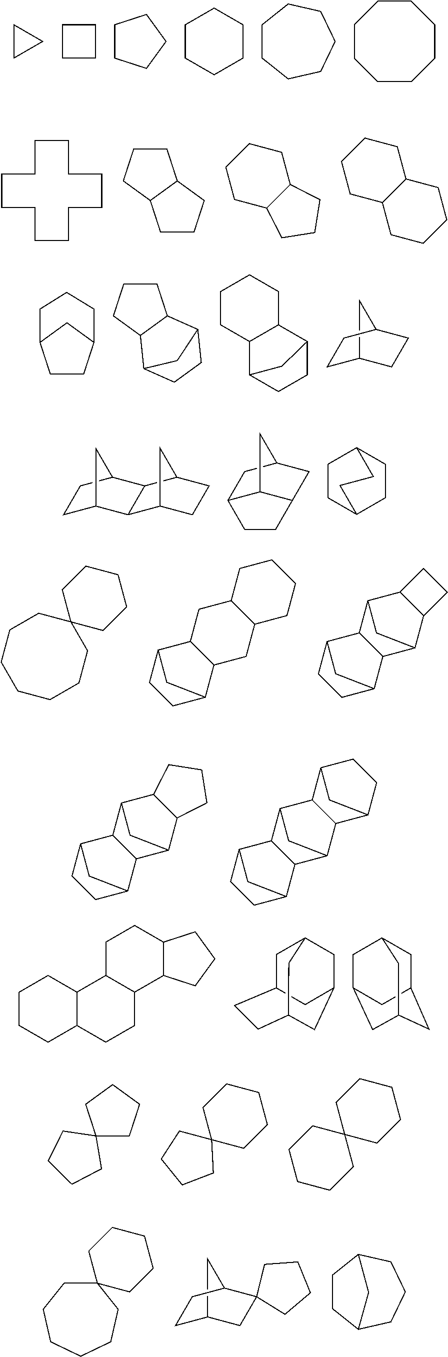

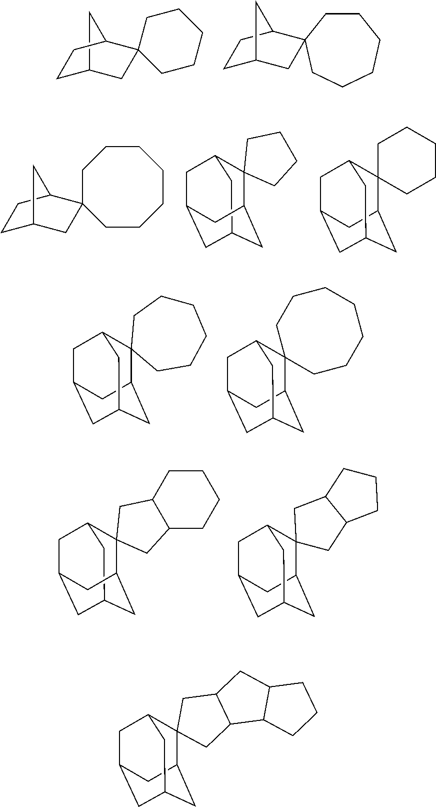

Specific examples of the alicyclic hydrocarbon group include a monocyclic cycloalkyl group such as a cyclopropyl group, a cyclobutyl group, a cyclopentyl group, a cyclohexyl group, a cycloheptyl group, a cyclooctyl group, a cyclononyl group, a cyclodecyl group and a cyclododecyl group, and a polycyclic cycloalkyl group such as a norbornyl group, an adamantly group, and a decahydronaphthyl group. The number of carbon atoms of the alicyclic hydrocarbon group may be 3 to 24, is preferably 3 to 18, is more preferably 3 to 16, is further preferably 3 to 12, and is further more preferably 3 to 10.

Examples of the aromatic hydrocarbon group include an aryl group such as a phenyl group, a naphthyl group, a biphenyl group, an anthryl group and a phenanthryl group. The number of carbon atoms of the aromatic hydrocarbon group may be 6 to 24, is preferably 6 to 18, is more preferably 6 to 14, and is further preferably 6 to 10.

Examples of the combined group include a combined group of an aromatic hydrocarbon group and a chain hydrocarbon group (such as an aromatic hydrocarbon group-alkanediyl group-*, an alkyl group-aromatic hydrocarbon group-* and an alkyl group-aromatic hydrocarbon group -alkanediyl group-*), a combined group of an alicyclic hydrocarbon group and a chain hydrocarbon group (such as an alicyclic hydrocarbon group-alkanediyl group-*, an alkyl group-alicyclic hydrocarbon group-* and an alkyl group-alicyclic hydrocarbon group-alkanediyl group-*), and a combined group of an aromatic hydrocarbon group and an alicyclic hydrocarbon group (such as an aromatic hydrocarbon group-alicyclic hydrocarbon group-* and an alicyclic hydrocarbon group-aromatic hydrocarbon group-*). * represents a bonding site.

Examples of the aromatic hydrocarbon group-alkanediyl group-* include an aralkyl group such as a benzyl group and a phenethyl group.

Examples of the alkyl group-aromatic hydrocarbon group-* include a tolyl group, a xylyl group, and a cumenyl group.

Examples of the alicyclic hydrocarbon group-alkanediyl group-* include a cycloalkylalkyl group such as a cyclohexylmethyl group, a cyclohexylethyl group, a 1-(adamantan-1-yl)methyl group and a 1-(adamantan-1-yl)-1-methylethyl group.

Examples of the alkyl group-alicyclic hydrocarbon group-* include a cycloalkyl group having an alkyl group such as a methylcyclohexyl group, a dim ethylcyclohexyl group and a 2-alkyladamantan-2-yl group.

Examples of the aromatic hydrocarbon group-alicyclic hydrocarbon group-* include a phenylcyclohexyl group.

Examples of the alicyclic hydrocarbon group-aromatic hydrocarbon group-* include a cyclohexylphenyl group.

In the combination, two or more types of alicyclic hydrocarbon groups, aromatic hydrocarbon groups, and chain hydrocarbon groups may each be combined. In the combined group, any group may be bonded to X.

When —CH₂— contained in the hydrocarbon group in R² is replaced with —O—, —CO—, —S—, —SO—, —NR⁷— or —SO₂—, the number of carbon atoms before replacement corresponds to the defined total number of carbon atoms of the hydrocarbon group. The number thereof may be one, or may be two or more.

Examples of the group formed by replacing —CH₂— contained in the hydrocarbon group with —O—, —CO—, —S—, —SO—, —NR⁷— or —SO₂— include the same as those exemplified in the present specification, within the range permitted by the upper limit of the number of carbon atoms.

Examples of the substituent that the hydrocarbon group in R² optionally has include a halogen atom, a cyano group and a nitro group.

When R² is a combined group of an alicyclic hydrocarbon group or an aromatic hydrocarbon group and a chain hydrocarbon group such as alkyl group, the chain hydrocarbon group such as alkyl group can substantially be a substituent for the alicyclic hydrocarbon group or the aromatic hydrocarbon group. When —CH₂— contained in the chain hydrocarbon group of R², such as alkyl group, is replaced with —O—, —S—, —SO₂—, —SO—, —NR⁷— or —CO—, R² can substantially have a substituent such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group and an alkyl peptide group.

Examples of the halogen atom include the same groups as described above.

Examples of the substituent include a halogen atom, an alkyl group having 1 to 6 carbon atoms or a haloalkyl group having 1 to 6 carbon atoms, where —CH₂— contained in the alkyl group is optionally replaced with —O— or —CO—, and it is preferably a halogen atom, an alkyl group having 1 to 4 carbon atoms or a haloalkyl group having 1 to 4 carbon atoms, where —CH₂— contained in the alkyl group is optionally replaced with —O— or —CO—, is more preferably a halogen atom, an alkyl group having 1 to 4 carbon atoms or a perfluoroalkyl group having 1 to 4 carbon atoms, and is further preferably a fluorine atom, an iodine atom, a methyl group, a t-butyl group or a trifluoro methyl group.

The hydrocarbon group having 1 to 36 carbon atoms represented by R² optionally has one substituent or a plurality of substituents.

Two R² may be bonded to each other to form a ring together with a sulfur atom to which they are bonded, and —CH₂— contained in the ring is optionally replaced with —O—, —S—, —CO—, —SO—, —NR⁷— or —SO₂—.

R² may be a chain hydrocarbon group having 1 to 18 carbon atoms and optionally having a substituent, where —CH₂— contained in the chain hydrocarbon group is optionally replaced with —O—, —S—, —SO₂— or —CO—, and the substituent or the chain hydrocarbon group may be bonded to each other to form a ring together with a sulfur atom to which they are bonded, a cyclic hydrocarbon group having 3 to 24 carbon atoms and optionally having a substituent, where —CH₂— contained in the cyclic hydrocarbon group is optionally replaced with —O—, —S—, —SO₂— or —CO—, and the substituent or the cyclic hydrocarbon group may be bonded to each other to form a ring together with a sulfur atom to which they are bonded, or a combined group of a cyclic hydrocarbon group having 3 to 24 carbon atoms and optionally having a substituent and a chain hydrocarbon group having 1 to 18 carbon atoms and optionally having a substituent, where —CH₂— contained in the cyclic hydrocarbon group and the chain hydrocarbon group is optionally replaced with —O—, —S—, —SO₂— or —CO—, and the substituent or the combined group may be bonded to each other to form a ring together with a sulfur atom to which they are bonded, is preferably a chain hydrocarbon group having 1 to 8 carbon atoms and optionally having a substituent, where —CH₂— contained in the chain hydrocarbon group is optionally replaced with —O—, —S—, —SO₂— or —CO—, and the substituent or the chain hydrocarbon group may be bonded to each other to form a ring together with a sulfur atom to which they are bonded, a cyclic hydrocarbon group having 3 to 18 carbon atoms and optionally having a substituent, where —CH₂— contained in the cyclic hydrocarbon group is optionally replaced with —O—, —S—, —SO₂— or —CO—, and the substituent or the cyclic hydrocarbon group may be bonded to each other to form a ring together with a sulfur atom to which they are bonded, or a combined group of a cyclic hydrocarbon group having 3 to 18 carbon atoms and optionally having a substituent and a chain hydrocarbon group having 1 to 8 carbon atoms and optionally having a substituent, where —CH₂— contained in the cyclic hydrocarbon group and the chain hydrocarbon group is optionally replaced with —O—, —S—, —SO₂— or —CO—, and the substituent or the combined group may be bonded to each other to form a ring together with a sulfur atom to which they are bonded, is more preferably an aromatic hydrocarbon group having 6 to 18 carbon atoms and optionally having a substituent, where the substituent or the aromatic hydrocarbon group may be bonded to each other to form a ring together with a sulfur atom to which they are bonded, or a combined group of an aromatic hydrocarbon group having 6 to 18 carbon atoms and optionally having a substituent and a chain hydrocarbon group having 1 to 6 carbon atoms and optionally having a substituent, where —CH₂— contained in the chain hydrocarbon group is optionally replaced with —O— or —CO—, and the substituent or the combined group may be bonded to each other to form a ring together with a sulfur atom to which they are bonded, and is further preferably a phenyl group optionally having a substituent, where the substituent or the phenyl group may be bonded to each other to form a ring together with a sulfur atom to which they are bonded, or a combined group of a phenyl group optionally having a substituent and an alkyl group having 1 to 4 carbon atoms and optionally having a substituent, where —CH₂— contained in the alkyl group is optionally replaced with —O— or —CO—, and the substituent or the combined group may be bonded to each other to form a ring together with a sulfur atom to which they are bonded. Here, —CH₂— contained in the ring is optionally replaced with —O—, —S—, —CO—, —SO— or —SO₂.

Examples of the halogen atom of R³ include a fluorine atom, a chlorine atom, a bromine atom and an iodine atom.

Examples of the hydrocarbon group having 1 to 18 carbon atoms of R³ include a chain hydrocarbon group such as an alkyl group, an alicyclic hydrocarbon group, an aromatic hydrocarbon group, and a group formed by combining these groups.

Examples of the alkyl group include a straight-chain or branched alkyl group such as a methyl group, an ethyl group, a n-propyl group, an isopropyl group, a n-butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group. The number of carbon atoms of the chain hydrocarbon group is preferably 1 to 12, is more preferably 1 to 10, is further preferably 1 to 9, is even more preferably 1 to 8, is further more preferably 1 to 6, is still more preferably 1 to 4, and is still further preferably 1 to 3.

The alicyclic hydrocarbon group may be either monocyclic or polycyclic. Examples of the alicyclic hydrocarbon group include the groups represented as follows. The bonding site can be set to any position.

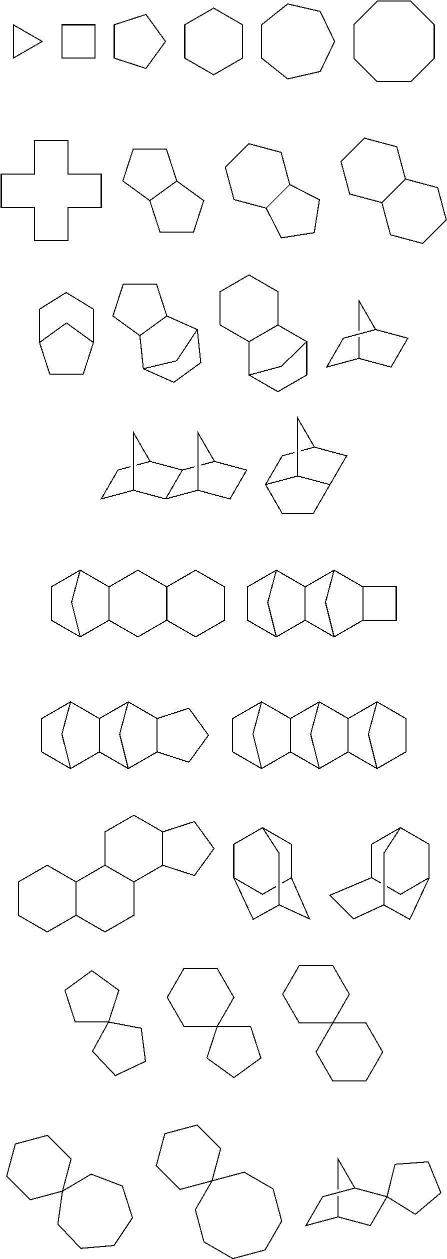

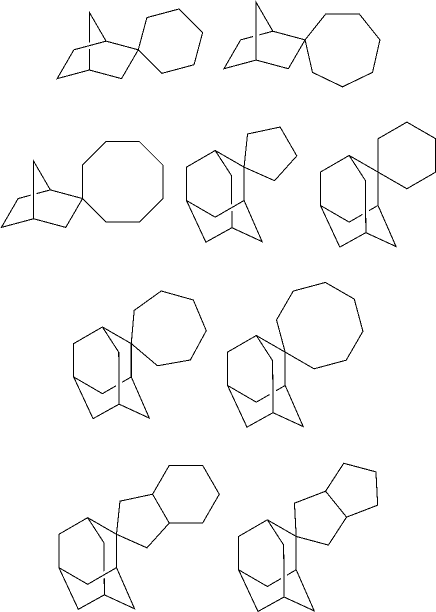

Specific examples of the alicyclic hydrocarbon group include a monocyclic cycloalkyl group such as a cyclopropyl group, a cyclobutyl group, a cyclopentyl group, a cyclohexyl group, a cycloheptyl group, a cyclooctyl group, a cyclononyl group, a cyclodecyl group and a cyclododecyl group, and a polycyclic cycloalkyl group such as a norbornyl group, an adamantly group, and a decahydronaphthyl group. The number of carbon atoms of the alicyclic hydrocarbon group is preferably 3 to 18, is more preferably 3 to 16, and is further preferably 3 to 12.

Examples of the aromatic hydrocarbon group include a phenyl group, a naphthyl group, a biphenyl group, an anthryl group, a phenanthryl group and a binaphthyl group. The number of carbon atoms of the aromatic hydrocarbon group is preferably 6 to 18, is more preferably 6 to 14, and is further preferably 6 to 10.

Examples of the group formed by combination include a combined group of an aromatic hydrocarbon group and a chain hydrocarbon group (such as an aromatic hydrocarbon group-alkanediyl group-* and an alkyl group-aromatic hydrocarbon group-*), a combined group of an alicyclic hydrocarbon group and a chain hydrocarbon group (such as an alicyclic hydrocarbon group-alkanediyl group-* and an alkyl group-alicyclic hydrocarbon group-*), and a combined group of an aromatic hydrocarbon group and an alicyclic hydrocarbon group (such as an aromatic hydrocarbon group-alicyclic hydrocarbon group-* and an alicyclic hydrocarbon group-aromatic hydrocarbon group-*). * represents a bonding site.

Examples of the aromatic hydrocarbon group-alkanediyl group-* include an aralkyl group such as a benzyl group and a phenethyl group.

Examples of the alkyl group-aromatic hydrocarbon group-* include a tolyl group, a xylyl group, and a cumenyl group.

Examples of the alicyclic hydrocarbon group-alkanediyl group-* include a cycloalkylalkyl group such as a cyclohexylmethyl group, a cyclohexylethyl group, a 1-(adamantan-1-yl)methyl group and a 1-(adamantan-1-yl)-1-methylethyl group.

Examples of the alkyl group-alicyclic hydrocarbon group-* include a cycloalkyl group having an alkyl group such as a methylcyclohexyl group, a dim ethylcyclohexyl group and a 2-alkyladamantan-2-yl group.

Examples of the aromatic hydrocarbon group-alicyclic hydrocarbon group-* include a phenylcyclohexyl group.

Examples of the alicyclic hydrocarbon group-aromatic hydrocarbon group-* include a cyclohexylphenyl group.

In the combination, two or more types of alicyclic hydrocarbon groups, aromatic hydrocarbon groups, and chain hydrocarbon groups may each be combined. In addition, in the combined group, any group may be bonded to a benzene ring.

When —CH₂— contained in the hydrocarbon group is replaced with —O—, —CO—, —S—, —SO—, —NR⁷— or —SO₂—, the number of carbon atoms before replacement corresponds to the defined total number of carbon atoms of the hydrocarbon group. In addition, the number thereof may be one, or may be two or more.

Examples of the group formed by replacing —CH₂— contained in the hydrocarbon group with —O—, —S—, —CO—, —SO—, —NR⁷— or —SO₂— include the same as those exemplified in the present specification, within the range permitted by the number of carbon atoms.

Examples of the substituent that the hydrocarbon group of R³ optionally has include a halogen atom, a cyano group and a nitro group.

When R³ is a combined group of an alicyclic hydrocarbon group or an aromatic hydrocarbon group and a chain hydrocarbon group such as alkyl group, the chain hydrocarbon group such as alkyl group can substantially be a substituent for the alicyclic hydrocarbon group or the aromatic hydrocarbon group. In addition, when —CH₂— contained in the chain hydrocarbon group of R³, such as alkyl group, is replaced with —O—, —S—, —SO₂—, —SO—, —NR⁷— or —CO—, R³ can substantially have a substituent such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group and an alkyl peptide group.

Examples of the halogen atom include the same groups as described above.

The hydrocarbon group optionally has one substituent or a plurality of substituents.

When R³ is a hydrocarbon group having a halogen atom, examples of the hydrocarbon group having a halogen atom of R³ include a chain hydrocarbon group having a halogen atom such as a haloalkyl group, an alicyclic hydrocarbon group having a halogen atom, and an aromatic hydrocarbon group having a halogen atom.

Examples of the alkyl group having a halogen atom include a haloalkyl group having 1 to 12 carbon atoms such as a fluorinated alkyl group having 1 to 12 carbon atoms, a chlorinated alkyl group having 1 to 12 carbon atoms, a brominated alkyl group having 1 to 12 carbon atoms, and an iodinated alkyl group having 1 to 12 carbon atoms. Examples of the haloalkyl group include a perfluoroalkyl group having 1 to 12 carbon atoms (such as a trifluoromethyl group, a pentafluoroethyl group, a heptafluoropropyl group and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group and an iodomethyl group. The number of carbon atoms of the haloalkyl group is preferably 1 to 9, is more preferably 1 to 6, is further preferably 1 to 4, and is further more preferably 1 to 3.

The —CH₂— contained in the haloalkyl group is optionally replaced with —O—, —CO— or the like, and examples of such a group include a haloalkoxy group, a haloalkoxycarbonyl group, a haloalkylcarbonyl group and a haloalkylcarbonyloxy group. Specific examples thereof include a haloalkoxy group having 1 to 11 carbon atoms, a haloalkoxycarbonyl group having 2 to 11 carbon atoms, a haloalkylcarbonyl group having 2 to 12 carbon atoms and a haloalkylcarbonyloxy group having 2 to 11 carbon atoms, such as a group in which one or more hydrogen atoms of the groups exemplified above are replaced with halogen atoms.

R³ is preferably a halogen atom, a haloalkyl group having 1 to 6 carbon atoms or an alkyl group having 1 to 6 carbon atoms, where —CH₂— contained in the haloalkyl group and the alkyl group is optionally replaced with —O— or —CO—, is more preferably a halogen atom, a haloalkyl group having 1 to 4 carbon atoms or an alkyl group having 1 to 4 carbon atoms, where —CH₂— contained in the alkyl group is optionally replaced with —O— or —CO—, is further preferably a halogen atom, a fluorinated alkyl group having 1 to 4 carbon atoms or an alkyl group having 1 to 4 carbon atoms, where —CH₂— contained in the alkyl group is optionally replaced with —O— or —CO—, is still more preferably a fluorine atom, an iodine atom, a perfluoroalkyl group having 1 to 4 carbon atoms or an alkyl group having 1 to 4 carbon atoms, and is still further preferably a fluorine atom, an iodine atom, a methyl group or a trifluoromethyl group.

m3 is preferably an integer of 0 to 3, and is more preferably 0, 1 or 2.

Examples of the cation of the salt (I) include the cations represented by the following formulas (I-c-1) to (I-c-50).

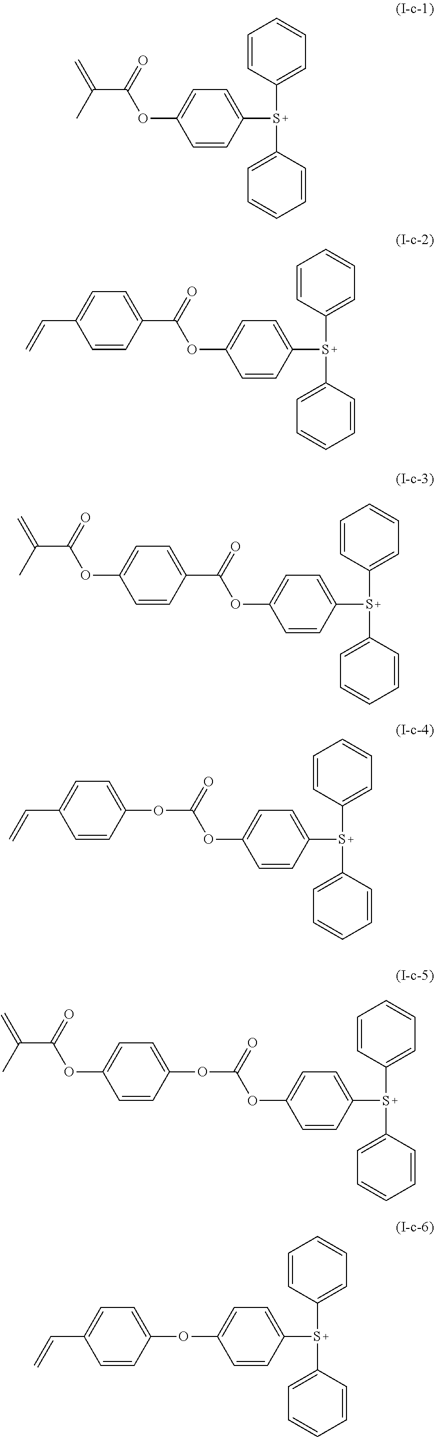

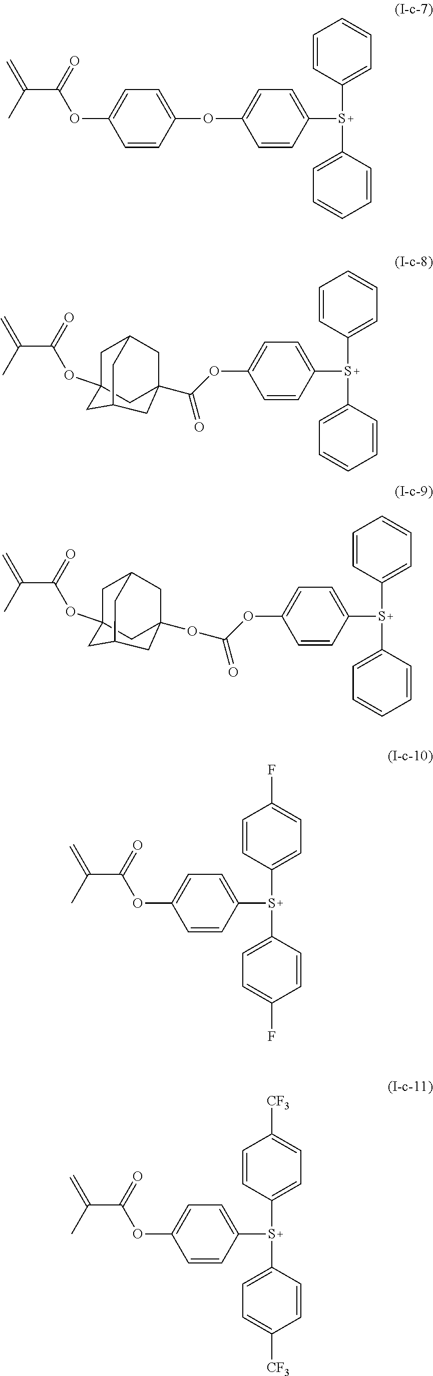

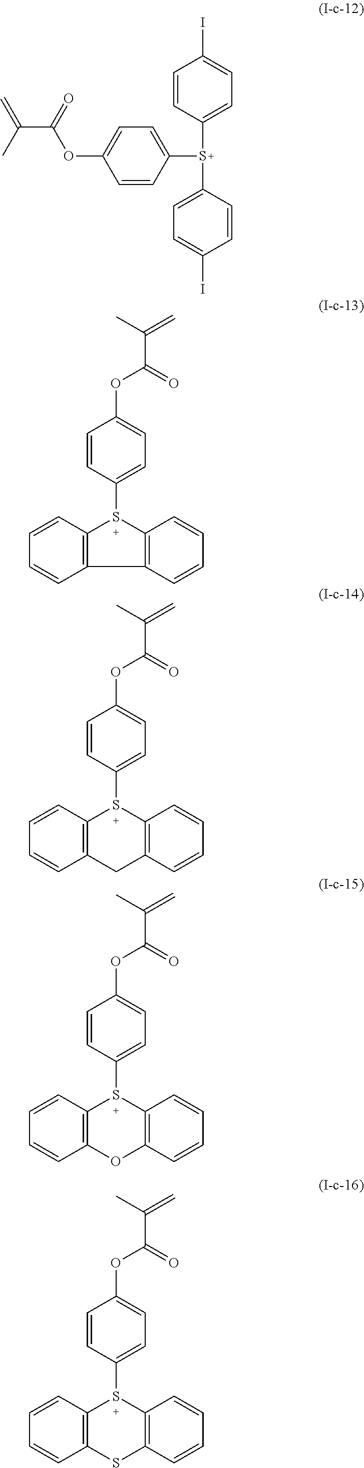

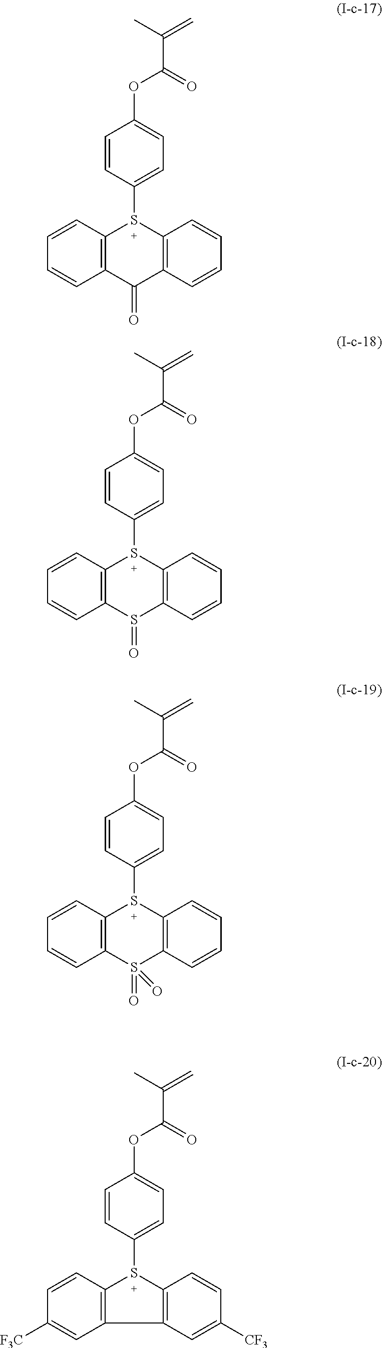

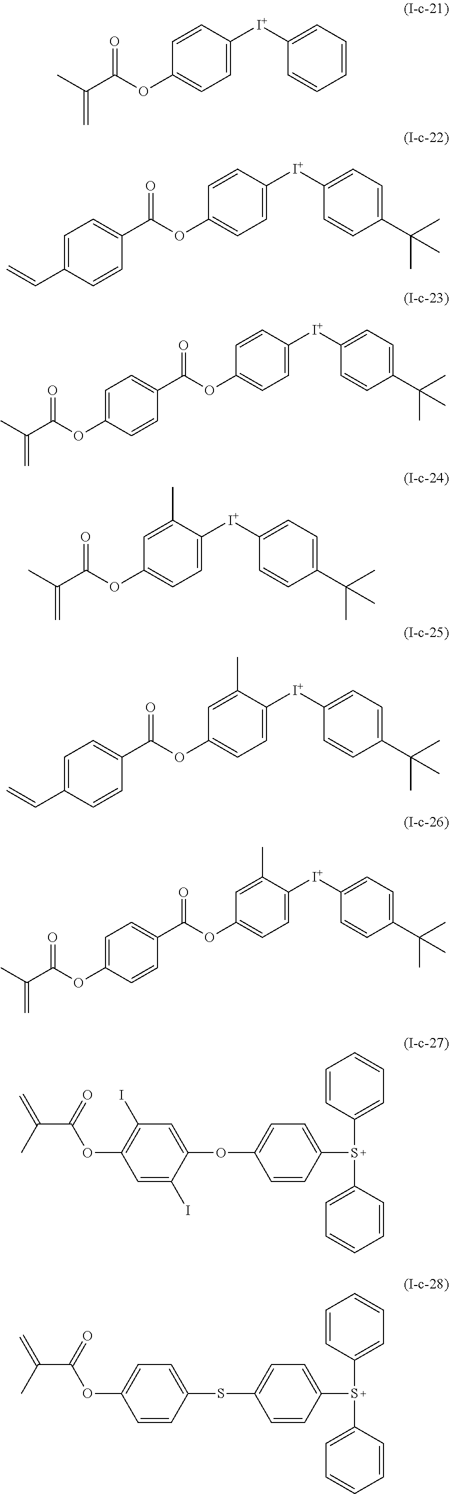

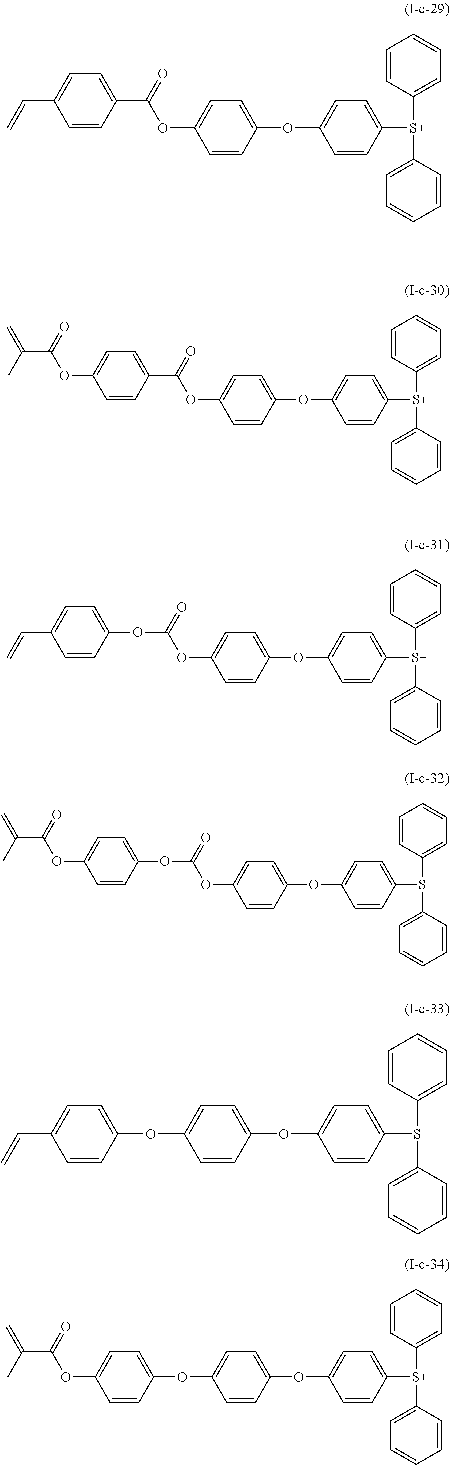

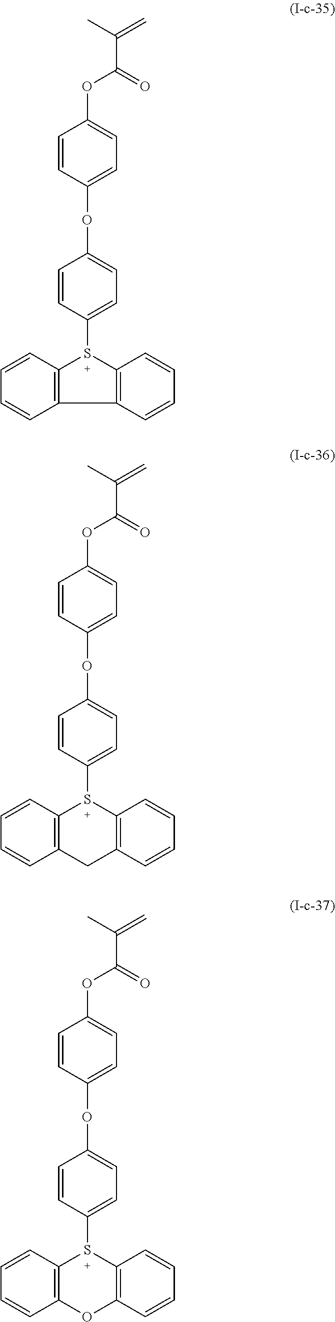

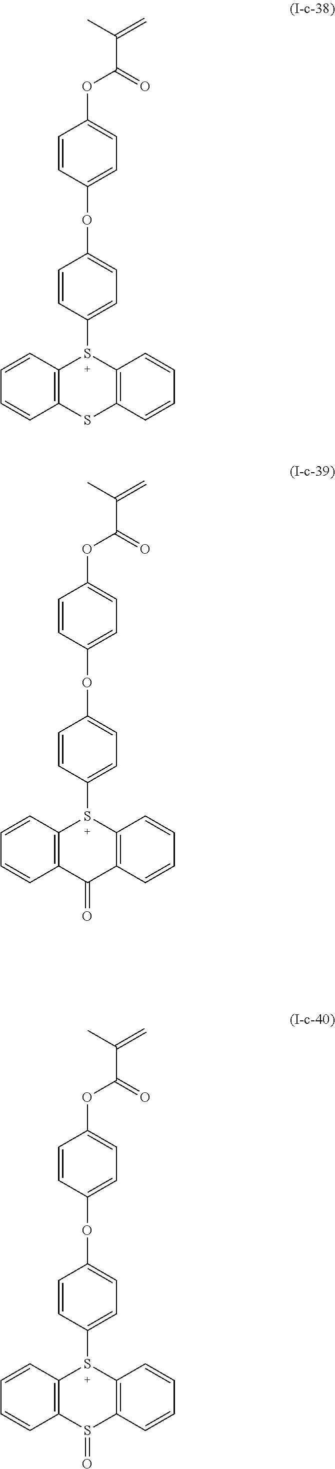

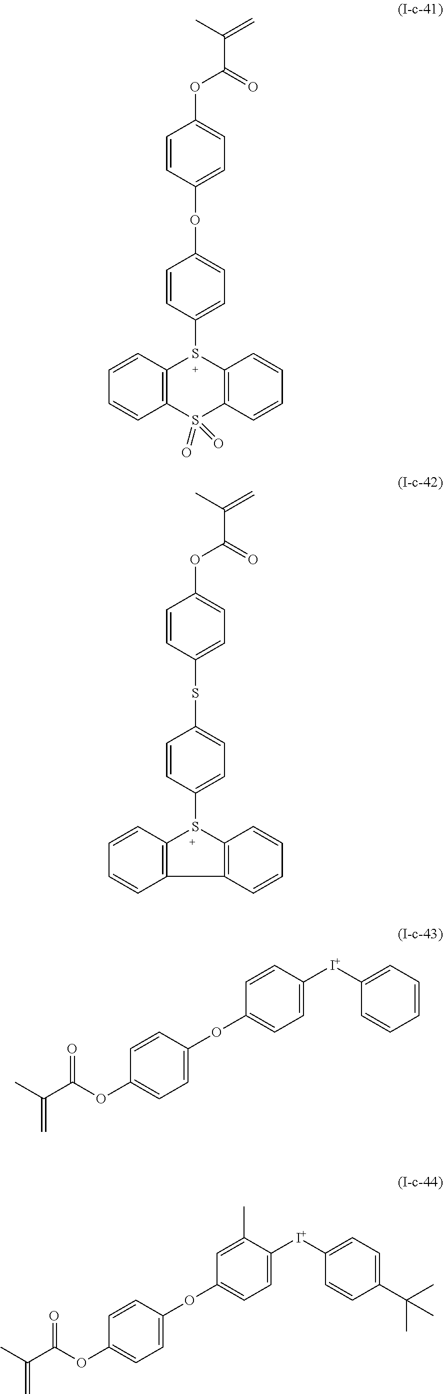

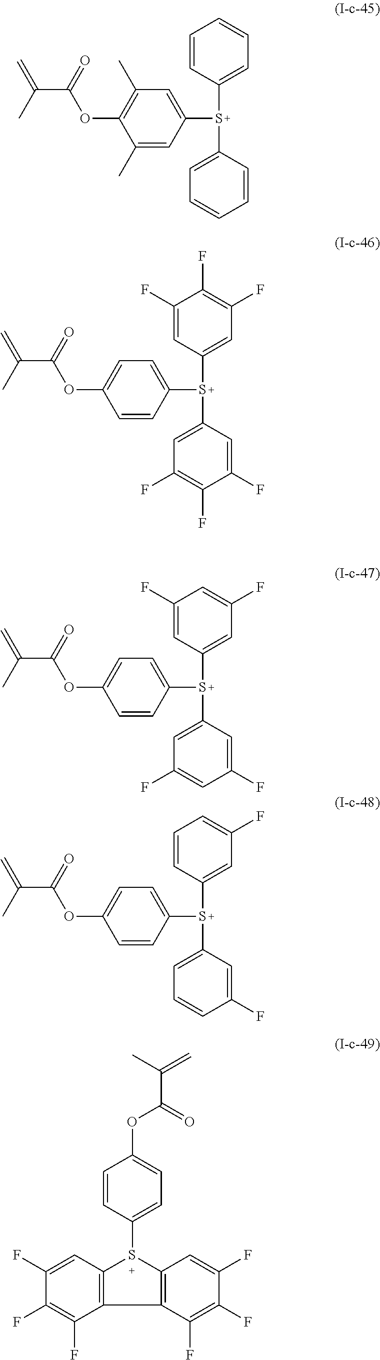

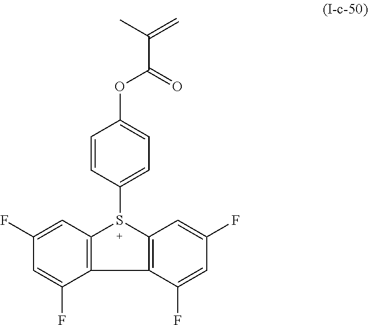

Specific examples of the salt (I) include salts in which the above-described cations and anions are arbitrarily combined. Specific examples of the salt (I) are shown in the following table.

In the following table, each symbol represents a symbol attached to a structure representing the anion or cation described above, and “to” indicates that the salts (I) correspond to the anions (I), respectively. For example, a salt (I-1) means a salt formed of an anion represented by formula (Ia-1) and a cation represented by formula (I-c-1); a salt (I-2) means a salt formed of an anion represented by formula (Ia-2) and a cation represented by formula (I-c-1); and a salt (I-101) means a salt formed of an anion represented by formula (Ia-1) and a cation represented by formula (I-c-2).

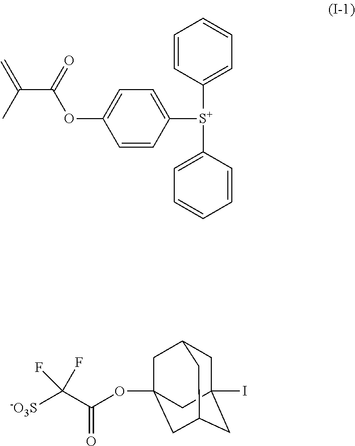

TABLE 1

| Salt (I) | Anion (I) | Cation (I) |
|---|---|---|
| (I-1) to (I-100) | (Ia-1) to (Ia-100) | (I-c-1) |
| (I-101) to (I-200) | (Ia-1) to (Ia-100) | (I-c-2) |
| (I-201) to (I-300) | (Ia-1) to (Ia-100) | (I-c-3) |
| (I-301) to (I-400) | (Ia-1) to (Ia-100) | (I-c-4) |
| (I-401) to (I-500) | (Ia-1) to (Ia-100) | (I-c-5) |
| (I-501) to (I-600) | (Ia-1) to (Ia-100) | (I-c-6) |
| (I-601) to (I-700) | (Ia-1) to (Ia-100) | (I-c-7) |
| (I-701) to (I-800) | (Ia-1) to (Ia-100) | (I-c-8) |
| (I-801) to (I-900) | (Ia-1) to (Ia-100) | (I-c-9) |
| (I-901) to (I-1000) | (Ia-1) to (Ia-100) | (I-c-10) |
| (I-1001) to (I-1100) | (Ia-1) to (Ia-100) | (I-c-11) |
| (I-1101) to (I-1200) | (Ia-1) to (Ia-100) | (I-c-12) |
| (I-1201) to (I-1300) | (Ia-1) to (Ia-100) | (I-c-13) |
| (I-1301) to (I-1400) | (Ia-1) to (Ia-100) | (I-c-14) |
| (I-1401) to (I-1500) | (Ia-1) to (Ia-100) | (I-c-15) |
| (I-1501) to (I-1600) | (Ia-1) to (Ia-100) | (I-c-16) |
| (I-1601) to (I-1700) | (Ia-1) to (Ia-100) | (I-c-17) |
| (I-1701) to (I-1800) | (Ia-1) to (Ia-100) | (I-c-18) |
| (I-1801) to (I-1900) | (Ia-1) to (Ia-100) | (I-c-19) |
| (I-1901) to (I-2000) | (Ia-1) to (Ia-100) | (I-c-20) |
| (I-2001) to (I-2100) | (Ia-1) to (Ia-100) | (I-c-21) |
| (I-2101) to (I-2200) | (Ia-1) to (Ia-100) | (I-c-22) |
| (I-2201) to (I-2300) | (Ia-1) to (Ia-100) | (I-c-23) |
| (I-2301) to (I-2400) | (Ia-1) to (Ia-100) | (I-c-24) |
| (I-2401) to (I-2500) | (Ia-1) to (Ia-100) | (I-c-25) |
| (I-2501) to (I-2600) | (Ia-1) to (Ia-100) | (I-c-26) |
| (I-2601) to (I-2700) | (Ia-1) to (Ia-100) | (I-c-27) |
| (I-2701) to (I-2800) | (Ia-1) to (Ia-100) | (I-c-28) |
| (I-2801) to (I-2900) | (Ia-1) to (Ia-100) | (I-c-29) |
| (I-2901) to (I-3000) | (Ia-1) to (Ia-100) | (I-c-30) |
| (I-3001) to (I-3100) | (Ia-1) to (Ia-100) | (I-c-31) |
| (I-3101) to (I-3200) | (Ia-1) to (Ia-100) | (I-c-32) |
| (I-3201) to (I-3300) | (Ia-1) to (Ia-100) | (I-c-33) |
| (I-3301) to (I-3400) | (Ia-1) to (Ia-100) | (I-c-34) |
| (I-3401) to (I-3500) | (Ia-1) to (Ia-100) | (I-c-35) |
| (I-3501) to (I-3600) | (Ia-1) to (Ia-100) | (I-c-36) |
| (I-3601) to (I-3700) | (Ia-1) to (Ia-100) | (I-c-37) |
| (I-3701) to (I-3800) | (Ia-1) to (Ia-100) | (I-c-38) |
| (I-3801) to (I-3900) | (Ia-1) to (Ia-100) | (I-c-39) |
| (I-3901) to (I-4000) | (Ia-1) to (Ia-100) | (I-c-40) |
| (I-4001) to (I-4100) | (Ia-1) to (Ia-100) | (I-c-41) |
| (I-4101) to (I-4200) | (Ia-1) to (Ia-100) | (I-c-42) |
| (I-4201) to (I-4300) | (Ia-1) to (Ia-100) | (I-c-43) |
| (I-4301) to (I-4400) | (Ia-1) to (Ia-100) | (I-c-44) |
| (I-4401) to (I-4500) | (Ia-1) to (Ia-100) | (I-c-45) |
| (I-4501) to (I-4600) | (Ia-1) to (Ia-100) | (I-c-46) |
| (I-4601) to (I-4700) | (Ia-1) to (Ia-100) | (I-c-47) |
| (I-4701) to (I-4800) | (Ia-1) to (Ia-100) | (I-c-48) |
| (I-4801) to (I-4900) | (Ia-1) to (Ia-100) | (I-c-49) |
| (I-4901) to (I-5000) | (Ia-1) to (Ia-100) | (I-c-50) |
| (I-5001) to (I-5050) | (Ia-101) | (I-c-1) to (I-c-50) |
| (I-5051) to (I-5100) | (Ia-102) | (I-c-1) to (I-c-50) |
| (I-5101) to (I-5150) | (Ia-103) | (I-c-1) to (I-c-50) |
| (I-5151) to (I-5200) | (Ia-104) | (I-c-1) to (I-c-50) |
| (I-5201) to (I-5250) | (Ia-105) | (I-c-1) to (I-c-50) |
| (I-5251) to (I-5300) | (Ia-106) | (I-c-1) to (I-c-50) |
| (I-5301) to (I-5350) | (Ia-107) | (I-c-1) to (I-c-50) |
| (I-5351) to (I-5400) | (Ia-108) | (I-c-1) to (I-c-50) |
| (I-5401) to (I-5450) | (Ia-109) | (I-c-1) to (I-c-50) |
| (I-5451) to (I-5500) | (Ia-110) | (I-c-1) to (I-c-50) |
| (I-5501) to (I-5550) | (Ia-111) | (I-c-1) to (I-c-50) |
| (I-5551) to (I-5600) | (Ia-112) | (I-c-1) to (I-c-50) |
| (I-5601) to (I-5650) | (Ia-113) | (I-c-1) to (I-c-50) |
| (I-5651) to (I-5700) | (Ia-114) | (I-c-1) to (I-c-50) |
| (I-5701) to (I-5750) | (Ia-115) | (I-c-1) to (I-c-50) |
| (I-5751) to (I-5800) | (Ia-116) | (I-c-1) to (I-c-50) |
| (I-5801) to (I-5850) | (Ia-117) | (I-c-1) to (I-c-50) |
| (I-5851) to (I-5900) | (Ia-118) | (I-c-1) to (I-c-50) |
| (I-5901) to (I-5950) | (Ia-119) | (I-c-1) to (I-c-50) |
| (I-5951) to (I-6000) | (Ia-120) | (I-c-1) to (I-c-50) |
| (I-6001) to (I-6050) | (Ia-121) | (I-c-1) to (I-c-50) |
| (I-6051) to (I-6100) | (Ia-122) | (I-c-1) to (I-c-50) |
| (I-6101) to (I-6150) | (Ia-123) | (I-c-1) to (I-c-50) |
| (I-6151) to (I-6200) | (Ia-124) | (I-c-1) to (I-c-50) |

Among them, the salt (I) is preferably a salt formed by combining an anion represented by any one of the formulas (Ia-1) to (Ia-12), (Ia-19), (Ia-22) to (Ia-50), and (Ia-101) to (Ia-120) with a cation represented by any one of the formulas (I-c-1) to (I-c-50).

### <Method for Producing Salt (I)>

The salt (I) can be produced by, for example, reacting a salt represented by formula (I-a) with a compound represented by formula (I-b) in a solvent in the presence of a catalyst, and then performing an acid treatment as necessary:

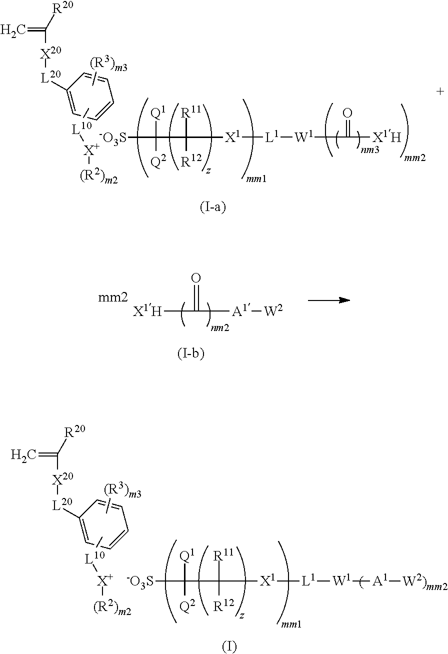

wherein symbols other than those described below represent the same meanings as described above, respectively;
X^{1′} represents —O— or —NR⁷—; nm2 and nm3 each independently represent 0 or 1;
A^{1′} represents a group obtained by removing —(CO)ₙₘ₂—X^{1′}—(CO)ₙₘ₃—(CO—X^{1′})ₙₘ₄— from A¹; and when nm2=nm3=0, nm4 represents 1, and when either nm2 or nm3 is 1, nm4 represents 0.

Examples of the solvent in this reaction include chloroform and acetonitrile.

Examples of the catalyst in this reaction include potassium carbonate, potassium iodide, pyridine, diethylaminopyridine, triethylamine, carbonyldiimidazole and 1-[3-(dimethylamino)propyl]-3-ethylcarbodiimide hydrochloride.

Examples of the acid in this reaction include hydrochloric acid, sulfuric acid and p-toluenesulfonic acid.

The reaction temperature is usually 5° C. to 80° C., and the reaction time is usually 0.5 hours to 24 hours.

Examples of the salt represented by formula (I-a) include the salts represented as follows, which can be easily produced by a known production method.

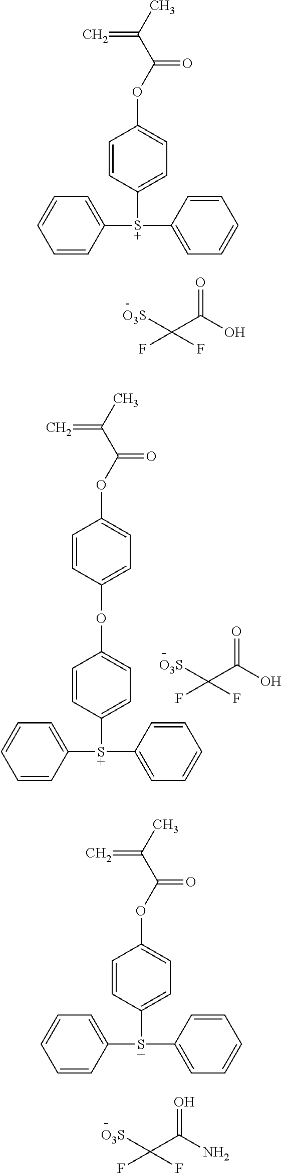

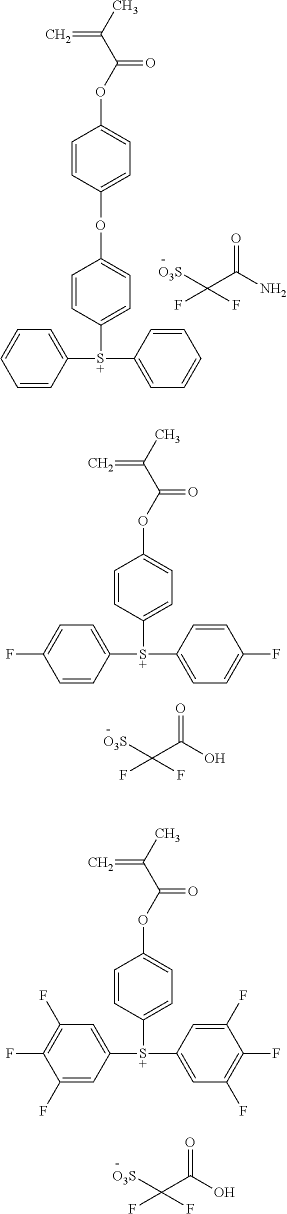

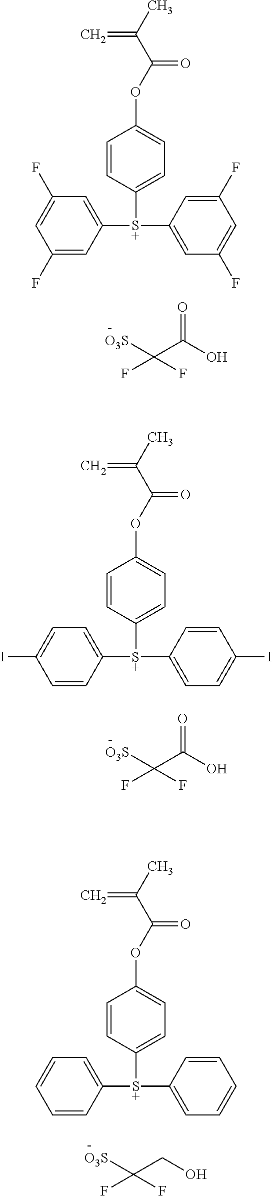

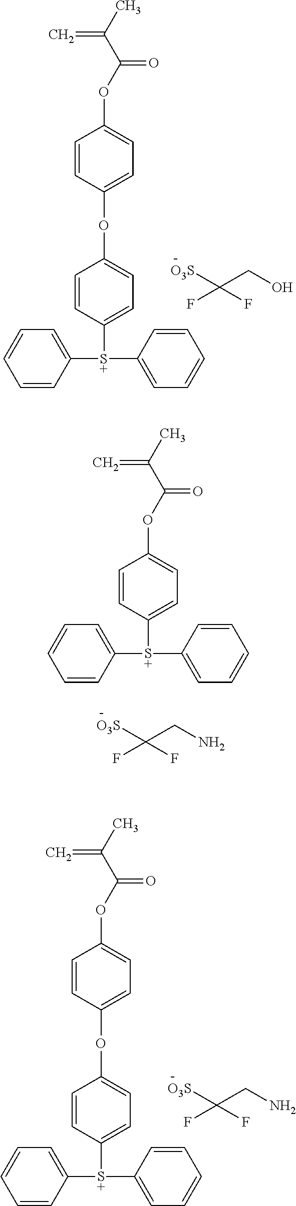

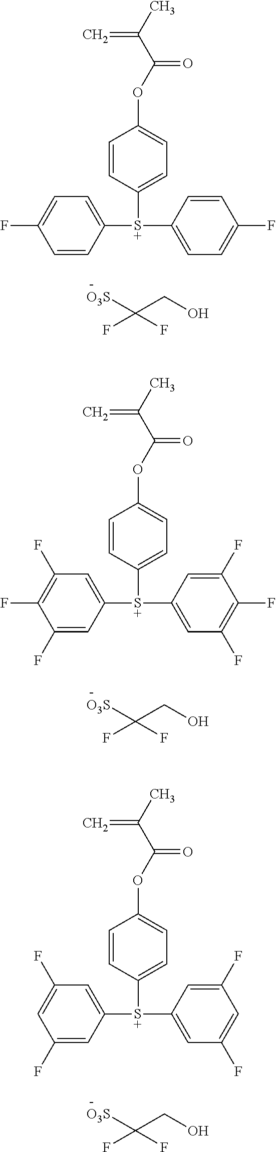

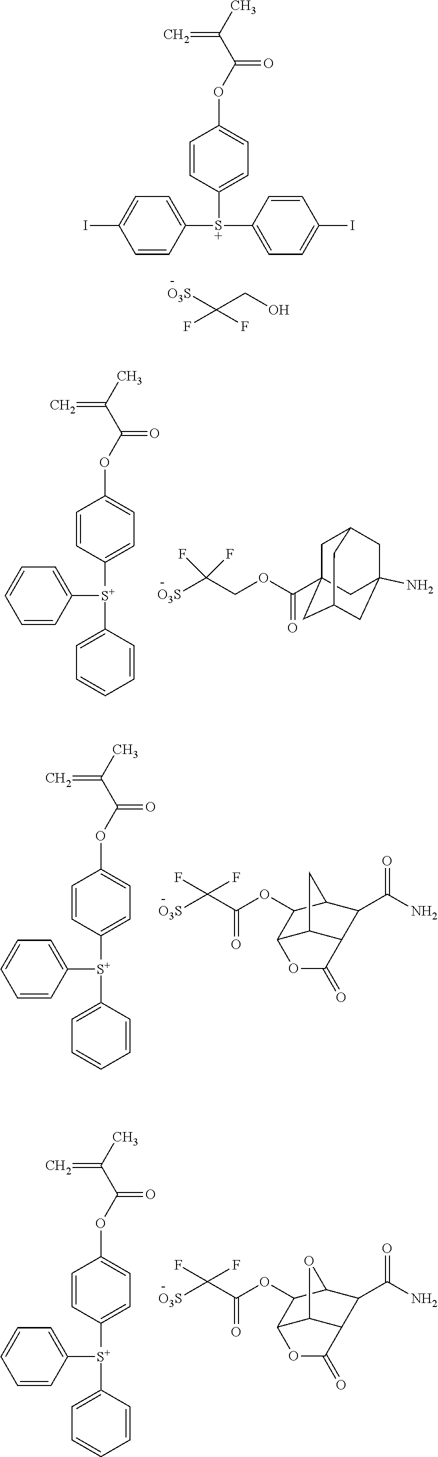

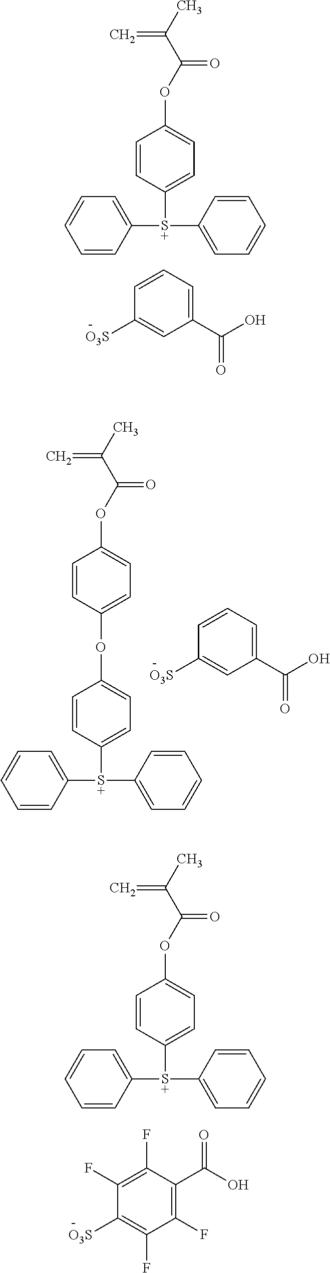

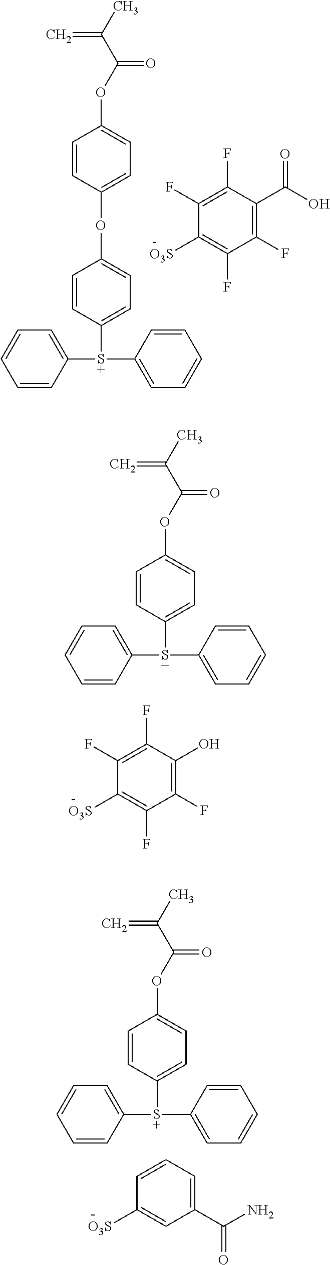

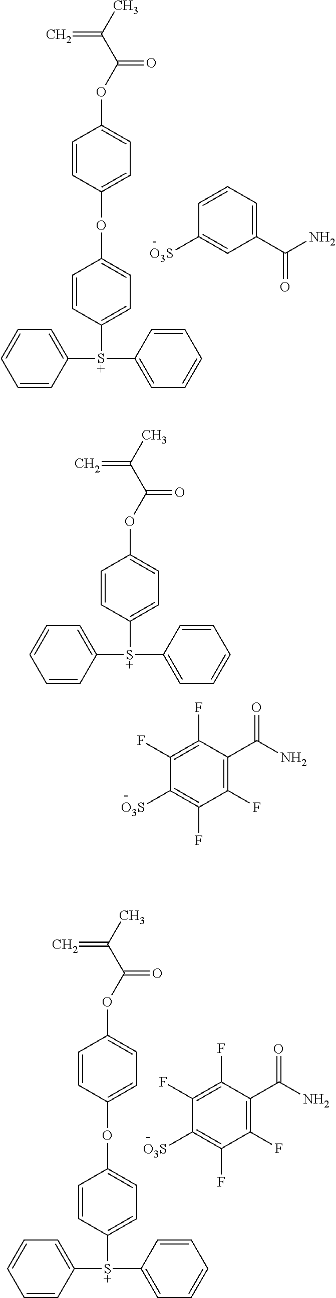

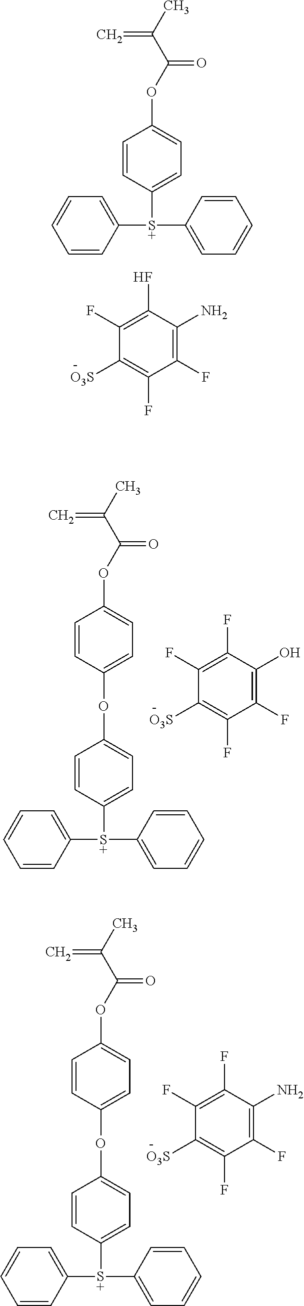

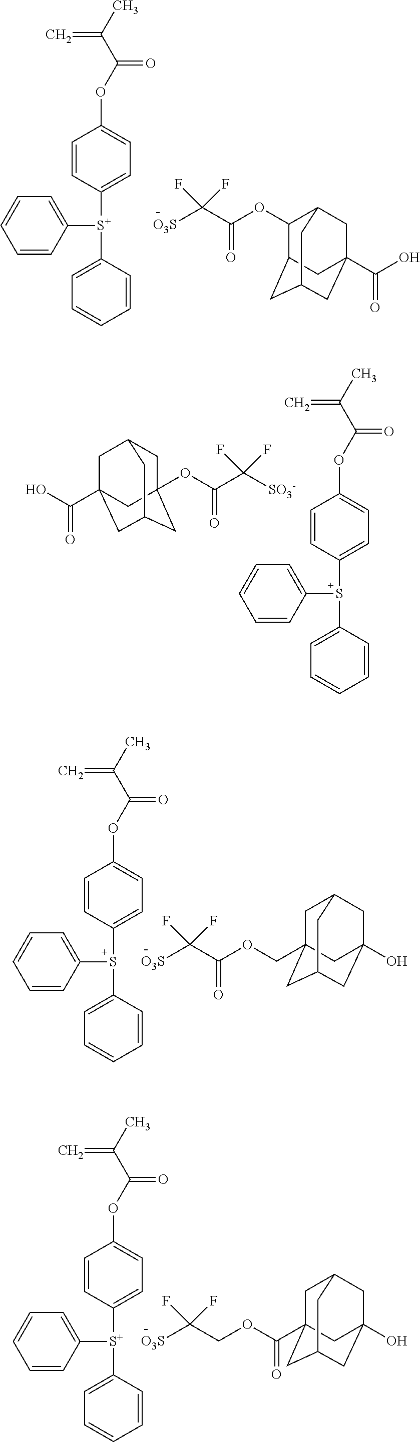

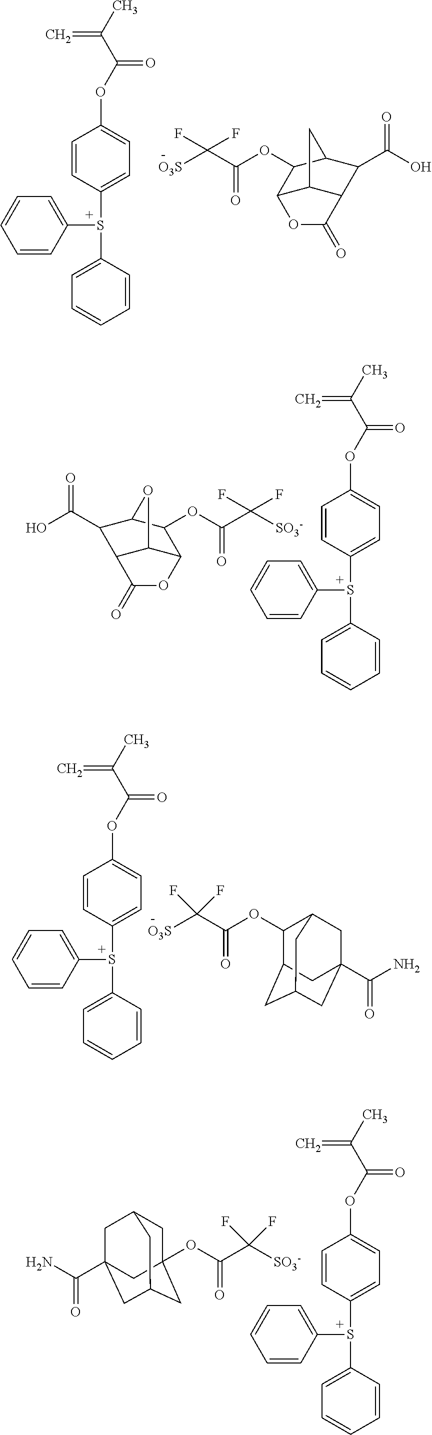

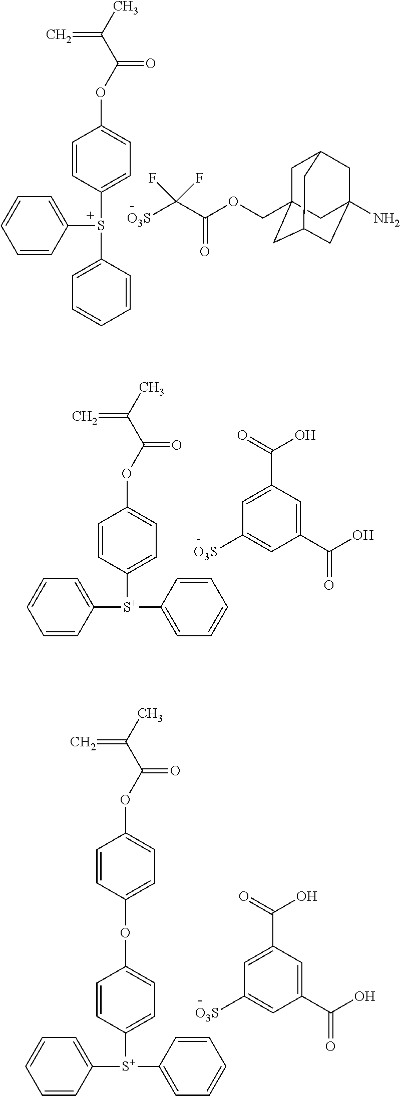

Examples of the compound represented by formula (I-b) include the compounds represented as follows, which are easily available on the market or can also be easily produced by a known production method.

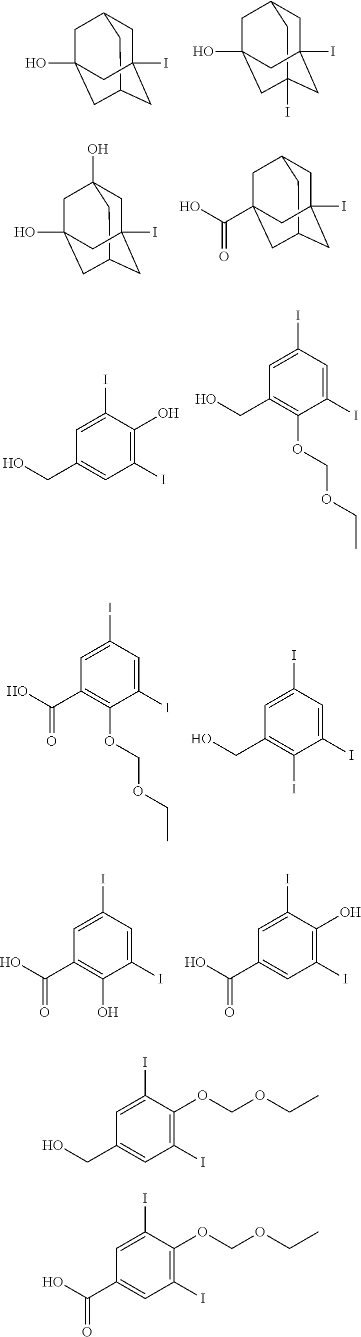

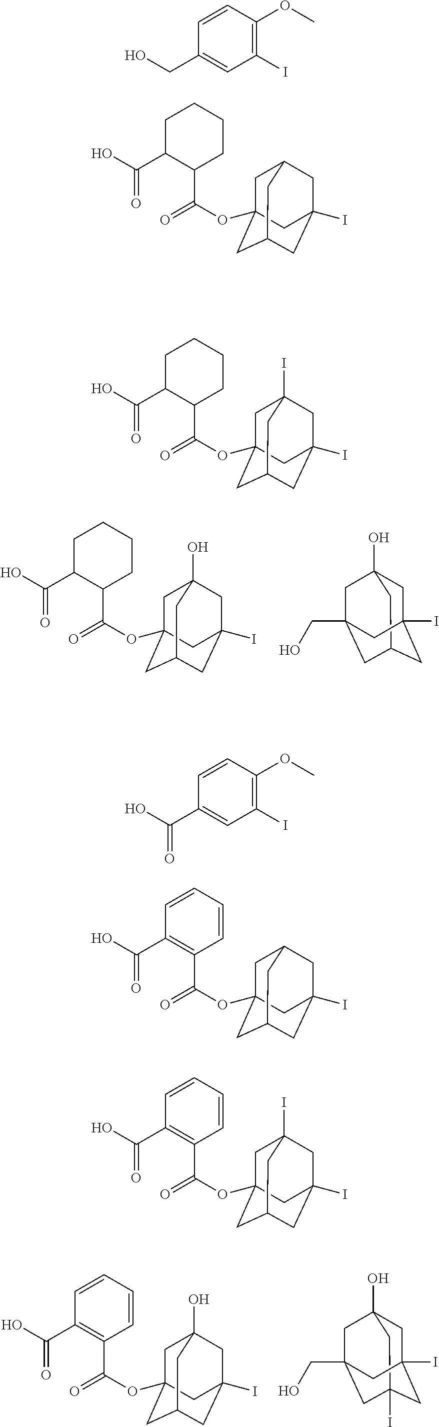

[Structural unit derived from salt represented by formula (I)] A structural unit derived from the salt represented by formula (I) shows a state where the double bond of CH₂═C—R²⁰ contained in the salt (I) is cleaved, and examples thereof include a structural unit represented by the following formula (IP) (hereinafter referred to as “structural unit (IP)” in some cases):

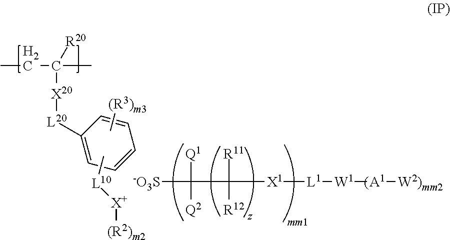

wherein all symbols have the same meanings as described above.

The structural unit (IP) functions as an acid generating agent similarly to the salt (I), and also functions as a structural unit constituting a compound or resin.

[Resin that includes structural unit (IP) derived from salt represented by formula (I)] The resin of the present invention is a resin that includes the structural unit (IP) (hereinafter referred to as “resin (Ap)” in some cases).

The resin (Ap) may be a homopolymer that includes one type of structural unit (IP), or may be a copolymer that includes two or more types of structural units (IP).

In addition, the resin (Ap) may include a structural unit other than the structural unit (IP). Examples of the structural unit other than the structural unit (IP) include, as will be described later, a structural unit having an acid labile group (hereinafter referred to as “structural unit (a1)” in some cases) and a structural unit other than the structural unit (a1). Examples of the structural unit other than the structural unit (a1) include a structural unit not having an acid labile group (hereinafter referred to as “structural unit (s)” in some cases) and a structural unit known in the field. Here, the term “acid labile group” refers to a group that has a leaving group, and in which the leaving group is detached due to contact with an acid, and the constitutional unit is converted into a constitutional unit having a hydrophilic group (for example, a hydroxy group or a carboxy group).

The content ratio of the structural unit (IP), with respect to all structural units of the resin (Ap), is, for example, 0.1 mol % or more, preferably 0.5 mol % or more, more preferably 0.8 mol % or more, and further preferably 1 mol % or more. In addition, the content ratio is, for example, 100 mol % or less, preferably 50 mol % or less, more preferably 30 mol % or less, and further preferably 20 mol % or less. Specifically, the content ratio is, for example, 0.1 to 100 mol %, preferably 0.5 to 50 mol %, more preferably 0.8 to 30 mol %, and further preferably 1 to 20 mol %.

In particular, when the resin (Ap) is used in a resist composition as will be described later, it is preferable to include the structural unit (a1) in addition to the structural unit (IP).

In addition, when the resin (Ap) is used in a resist composition as will be described later, it may be used in combination with a resin that includes the structural unit (a1) (hereinafter referred to as “resin (A)” in some cases) and/or a resin other than the resin (A), regardless of whether the resin (Ap) includes the structural unit (a1) or not. Hereinafter, the resin (Ap) and/or the resin (A) may be referred to as “resin (A) and/or the like”.

It is preferable that the resin (Ap) and the resin (A) each further include a structural unit other than the structural unit (a1).

<Structural Unit (a1)>

The structural unit (a1) is derived from a monomer having an acid labile group (hereinafter referred to as “monomer (a1)” in some cases).

The acid labile group included in the resin (A) and/or the like is preferably a group represented by formula (1) (hereinafter also referred to as group (1)) and/or a group represented by formula (2) (hereinafter also referred to as group (2)):

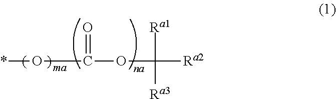

wherein R^{a1}, R^{a2} and R^{a3} each independently represent an alkyl group having 1 to 8 carbon atoms, an alkenyl group having 2 to 8 carbon atoms, an alicyclic hydrocarbon group having 3 to 20 carbon atoms, an aromatic hydrocarbon group having 6 to 18 carbon atoms, or a combined group of these groups, or R^{a1} and R^{a2} are bonded to each other to form an alicyclic hydrocarbon group having 3 to 20 carbon atoms together with a carbon atom to which they are bonded, and the alkyl group, the alkenyl group, the alicyclic hydrocarbon group, and the aromatic hydrocarbon group optionally have a halogen atom;
ma and na each independently represent 0 or 1, and at least one of ma and na represents 1; and
* represents a bonding site,

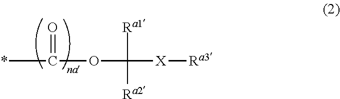

wherein R^{a1} and R^{a2′} each independently represent a hydrogen atom or a hydrocarbon group having 1 to 12 carbon atoms, R^{a3′} represents a hydrocarbon group having 1 to 20 carbon atoms, or R^{a2′} and R^{a3′} are bonded to each other to form a heterocyclic group having 3 to 20 carbon atoms together with a carbon atom to which they are bonded and X, and —CH₂— contained in the hydrocarbon group and the heterocyclic group is optionally replaced with —O— or —S—, and the hydrocarbon group and the heterocyclic group optionally have a halogen atom;
X represents an oxygen atom or a sulfur atom;
na′ represents 0 or 1; and
* represents a bonding site.

Examples of the alkyl group in R^{a1}, R^{a2} and R^{a3} include a methyl group, an ethyl group, a propyl group, a butyl group, a pentyl group, a hexyl group, a heptyl group and an octyl group.

Examples of the alkenyl group in R^{a1}, R^{a2} and R^{a3} include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The alicyclic hydrocarbon group in R^{a1}, R^{a2} and R^{a3} may be either monocyclic or polycyclic. Examples of the monocyclic alicyclic hydrocarbon group include a cycloalkyl group such as a cyclopentyl group, a cyclohexyl group, a cycloheptyl group and a cyclooctyl group. Examples of the polycyclic alicyclic hydrocarbon group include a decahydronaphthyl group, an adamantly group, a norbornyl group, and the following groups, where * represents a bonding site. The number of carbon atoms of the alicyclic hydrocarbon group of R^{a1}, R^{a2} and R^{a3} is preferably 3 to 16.

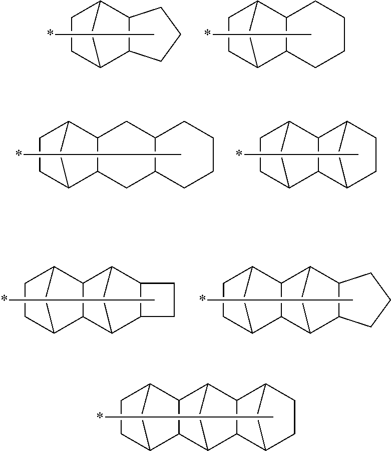

Examples of the aromatic hydrocarbon group in R^{a1}, R^{a2} and R^{a3} include an aryl group such as a phenyl group, a naphthyl group, an anthryl group, a biphenyl group and a phenanthryl group.

Examples of the combined group include: a combined group of the above-described alkyl group and alicyclic hydrocarbon group (for example, an alkylcycloalkyl group or cycloalkylalkyl group such as a methylcyclohexyl group, a dim ethylcyclohexyl group, a methylnorbornyl group, a cyclohexylmethyl group, an adamantylmethyl group, an adamantyldimethyl group and a norbornylethyl group); an aralkyl group such as a benzyl group; an aromatic hydrocarbon group having an alkyl group (such as a p-methylphenyl group, a p-tert-butylphenyl group, a tolyl group, a xylyl group, a cumenyl group, a mesityl group, a 2,6-diethylphenyl group and a 2-methyl-6-ethylphenyl group); an aromatic hydrocarbon group having an alicyclic hydrocarbon group (such as a p-cyclohexylphenyl group and a p-adamantylphenyl group); and an aryl-cycloalkyl group such as a phenylcyclohexyl group.

Preferably, ma is 0 and na is 1.

When R^{a1} and R^{a2} are bonded to each other to form an alicyclic hydrocarbon group, examples of —C(R^{a1})(R^{a2})(R^{a3}) include the following groups. The alicyclic hydrocarbon group preferably has 3 to 12 carbon atoms. * represents a bonding site to —O—.

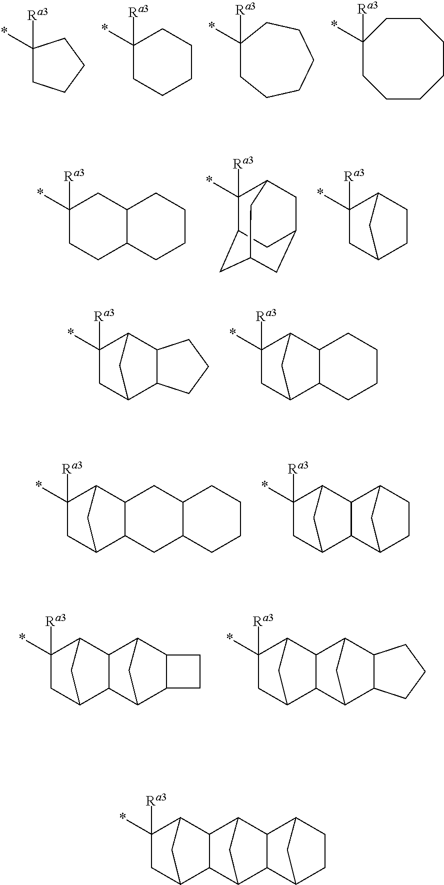

Examples of the hydrocarbon group in R^{a1′}, R^{a2′} and R^{a3′} include an alkyl group, an alicyclic hydrocarbon group, an aromatic hydrocarbon group, and a group formed by combining these groups.

Examples of the alkyl group, the alicyclic hydrocarbon group, the aromatic hydrocarbon group, and the group formed by combining these groups include the same groups as those listed for R^{a1}, R^{a2} and R^{a3}.

When R^{a2′} and R^{a3′} are bonded to each other to form a heterocyclic group together with a carbon atom to which they are bonded and X, examples of —C(R^{a1′})(R^{a2′})—X—R^{a3′} include the following groups. * represents a bonding site.

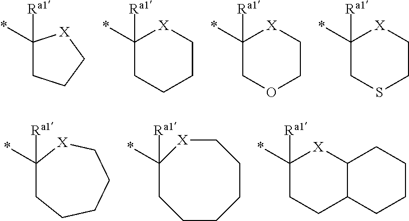

At least one of R^{a1′} and R^{a2′} is preferably a hydrogen atom.

na′ is preferably 0.

Examples of the halogen atom that R^{a1}, R^{a2}, R^{a3}, R^{a1}, R^{a2′} and R^{a3′} optionally have include a fluorine atom, a chlorine atom, a bromine atom and an iodine atom.

Examples of the group (1) include the following groups.

A group in which R^{a1}, R^{a2} and R^{a3} are alkyl groups, ma=0, and na=1 in formula (1). As the group, a tert-butoxycarbonyl group is preferable.

A group in which R^{a1} and R^{a2} form an adamantyl group together with a carbon atom to which they are bonded, R^{a3} is an alkyl group, ma=0, and na=1 in formula (1).

A group in which R^{a1} and R^{a2} are each independently an alkyl group, R^{a3} is an adamantyl group, ma=0, and na=1 in formula (1).

Specific examples of the group (1) include the following groups. * represents a bonding site.

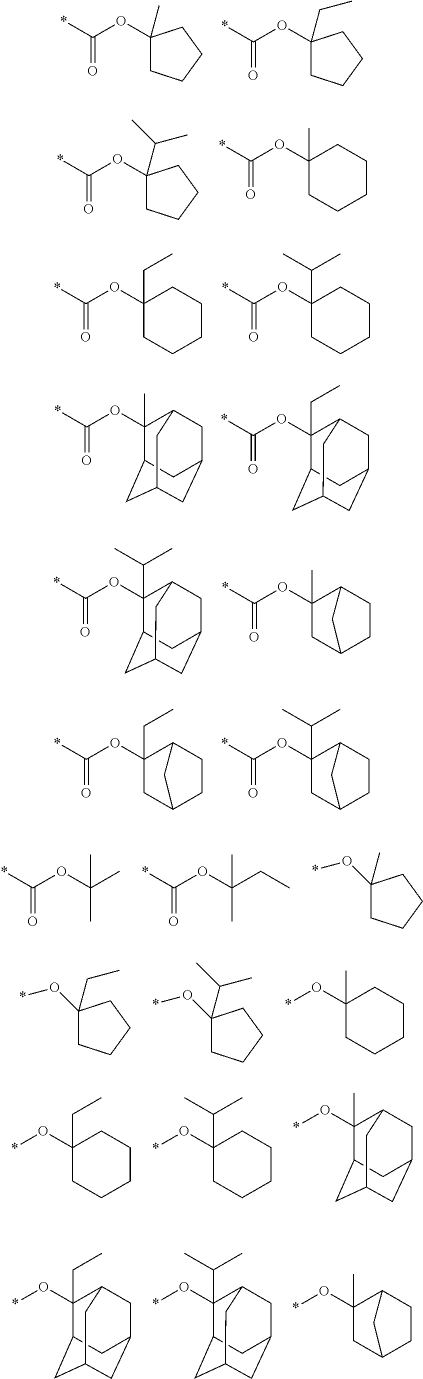

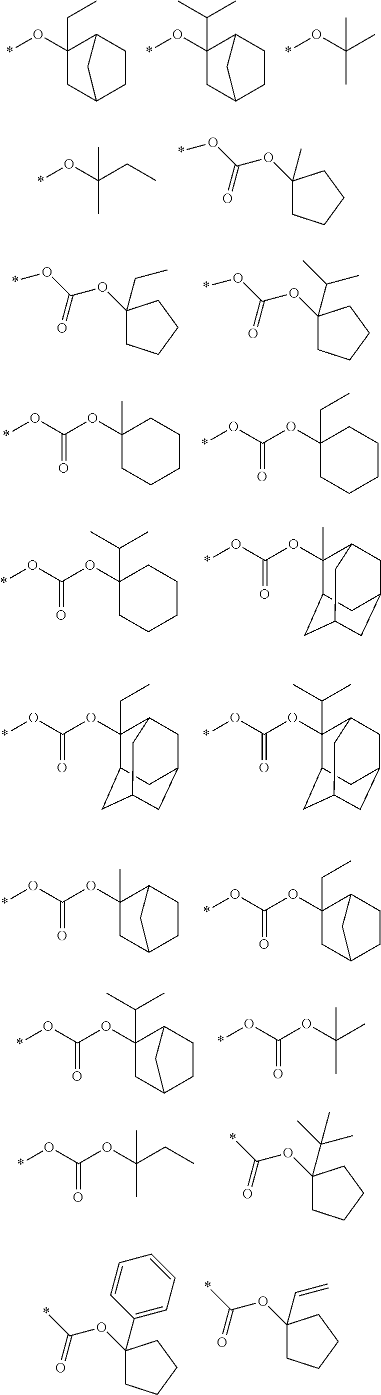

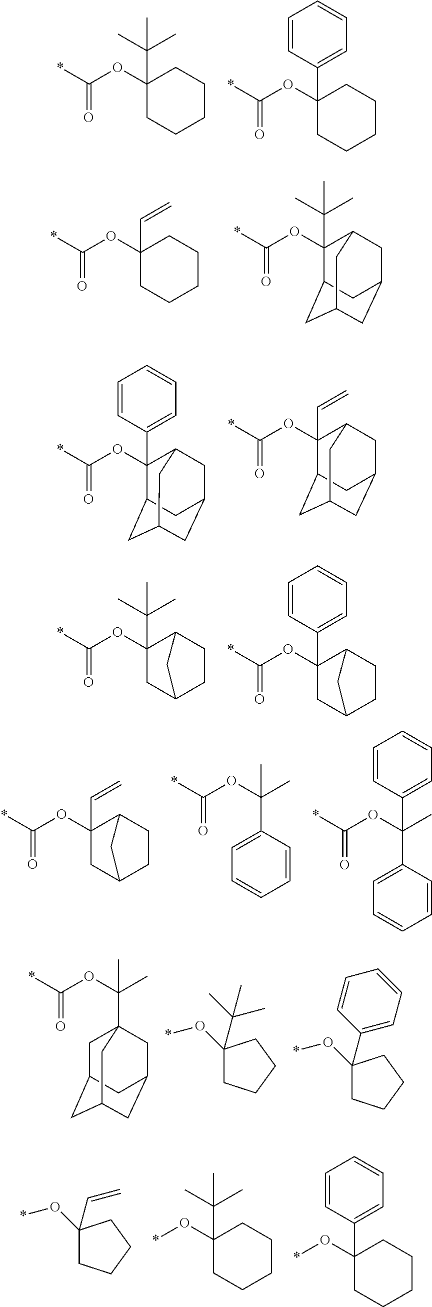

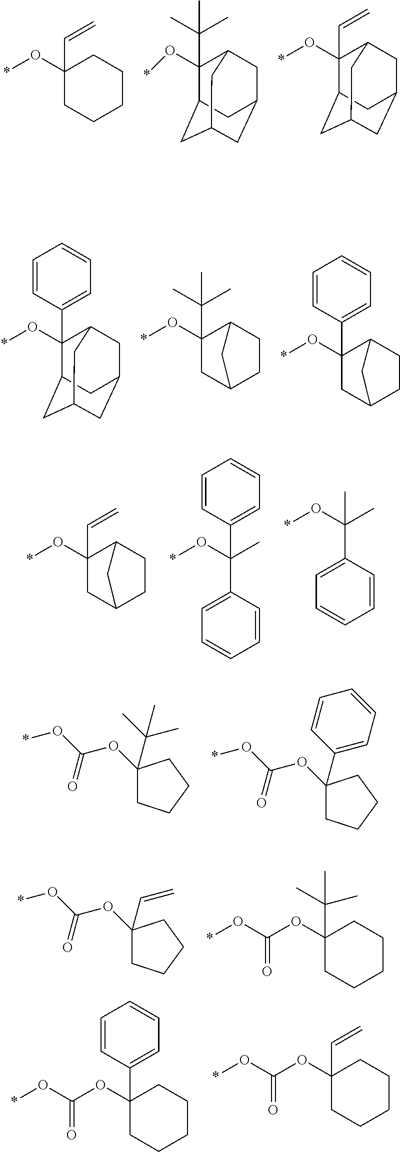

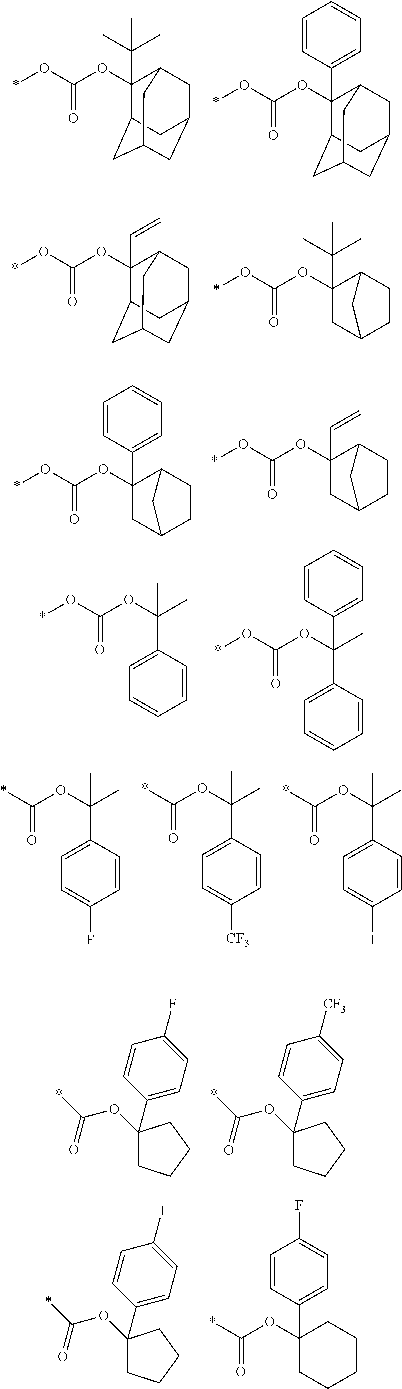

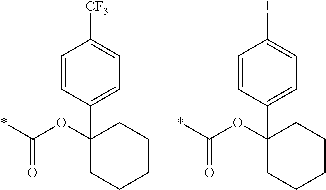

Specific examples of the group (2) include the following groups. * represents a bonding site.

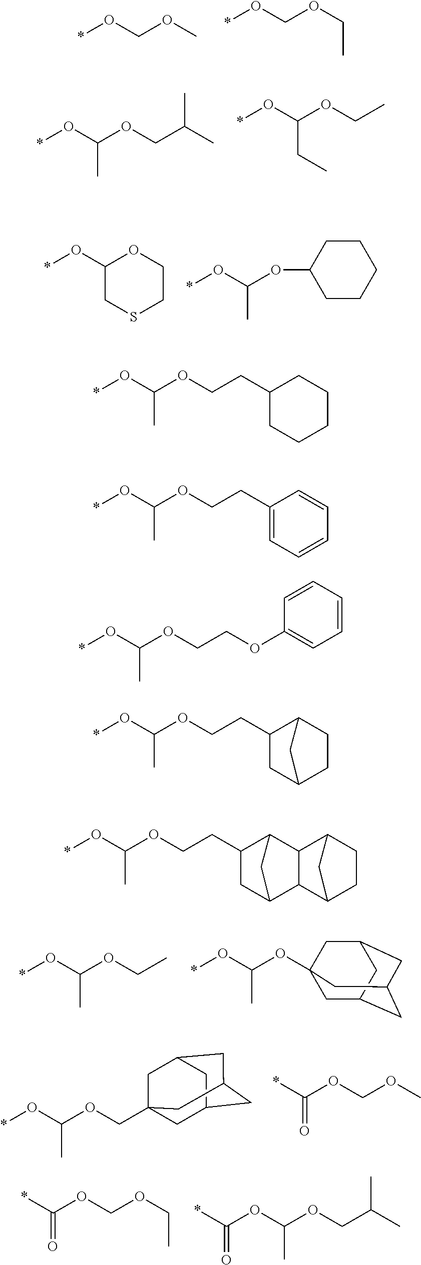

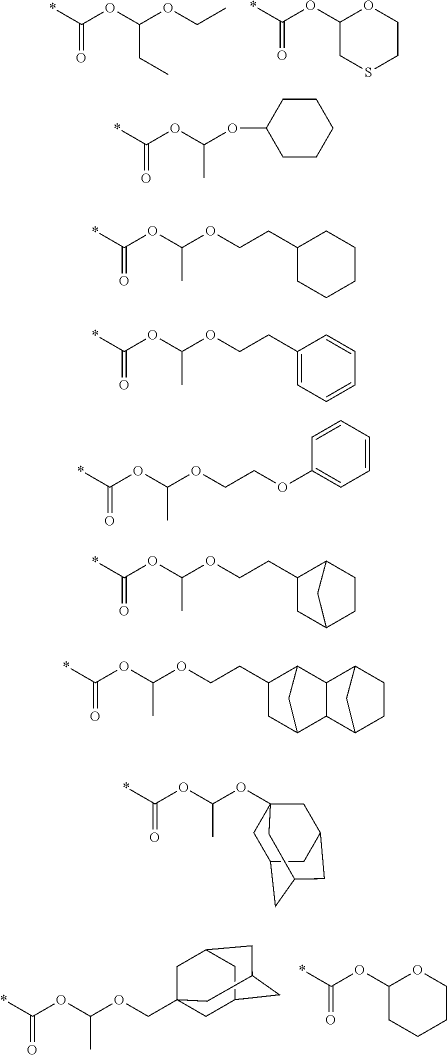

The monomer (a1) is preferably a monomer having an acid labile group and an ethylenically unsaturated bond, and is more preferably a (meth)acrylic monomer having an acid labile group.

Among the (meth)acrylic monomers having an acid labile group, preferable are those having an alicyclic hydrocarbon group having 5 to 20 carbon atoms. When the resin (A) and/or the like having a structural unit derived from the monomer (a1) having a bulky structure such as an alicyclic hydrocarbon group is used in a resist composition, the resolution of the resist pattern can be improved.

Examples of the structural unit derived from a (meth)acrylic monomer having the group (1) include a structural unit represented by formula (a1-0) (hereinafter referred to as structural unit (a1-0) in some cases), a structural unit represented by formula (a1-1) (hereinafter referred to as structural unit (a1-1) in some cases), or a structural unit represented by formula (a1-2) (hereinafter referred to as structural unit (a1-2) in some cases). It is preferably at least one structural unit selected from the group consisting of the structural unit (a1-0), the structural unit (a1-1), and the structural unit (a1-2), and is more preferably at least one or two structural units selected from the group consisting of the structural unit (a1-1) and the structural unit (a1-2). These may be used singly or in combination of two or more:

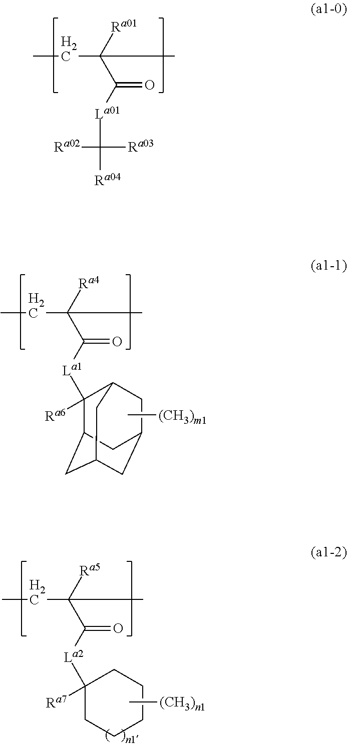

wherein all symbols in formulas (a1-0), (a1-1) and (a1-2) have the same meanings as described above.

Examples of the halogen atom in R^{a01}, R^{a4} and R^{a5} include a fluorine atom, a chlorine atom, a bromine atom and an iodine atom.

Examples of the alkyl group optionally having a halogen atom in R^{a01}, R^{a4} and R^{a5} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group and an iodomethyl group. The number of carbon atoms of the alkyl group is preferably 1 to 4, is more preferably 1 to 3, and is further preferably 1 or 2. The alkyl group is preferably a methyl group or an ethyl group, and is more preferably a methyl group.

R^{a01}, R^{a4} and R^{a5} are preferably hydrogen atoms or methyl groups, and are more preferably methyl groups.

L^{a01}, L^{a1} and L^{a2} are preferably oxygen atoms or *—O—(CH₂)ₖ₀₁—CO—O—, where k01 is preferably an integer of 1 to 4, and is more preferably 1, and are more preferably oxygen atoms.

Examples of the alkyl group, the alkenyl group, the alicyclic hydrocarbon group, the aromatic hydrocarbon group, and the combined group of these groups in R^{a02}, R^{a03}, R^{a04}, R^{a6} and R^{a7} include the same groups as those listed for R^{a1}, R^{a2} and R^{a3} in formula (1).

The alkyl group in R^{a02}, R^{a03}, and R^{a04} is preferably an alkyl group having 1 to 6 carbon atoms, is more preferably a methyl group or an ethyl group, and is further preferably a methyl group.

The alkyl group in R^{a6} and R^{a7} is preferably an alkyl group having 1 to 6 carbon atoms, is more preferably a methyl group, an ethyl group, an isopropyl group or a t-butyl group, and is further preferably an ethyl group, an isopropyl group or a t-butyl group.

The alkenyl group in R^{a6} and R^{a7} is preferably an alkenyl group having 2 to 6 carbon atoms, and is more preferably an ethenyl group, a propenyl group, an isopropenyl group or a butenyl group.

The number of carbon atoms of the alicyclic hydrocarbon group of R^{a02}, R^{a03}, R^{a04}, R^{a6} and R^{a7} is preferably 5 to 12, and is more preferably 5 to 10.

The number of carbon atoms of the aromatic hydrocarbon group of R^{a02}, R^{a03}, R^{a04}, R^{a6} and R^{a7} is preferably 6 to 12, and is more preferably 6 to 10.

In the case of a combined group of an alkyl group and an alicyclic hydrocarbon group, the total number of carbon atoms in the combination of the alkyl group and the alicyclic hydrocarbon group is preferably 18 or less.

In the case of a combined group of an alkyl group and an aromatic hydrocarbon group, the total number of carbon atoms in the combination of the alkyl group and the aromatic hydrocarbon group is preferably 18 or less.

R^{a02} and R^{a03} are preferably alkyl groups having 1 to 6 carbon atoms and optionally having a halogen atom or aromatic hydrocarbon groups having 6 to 12 carbon atoms and optionally having a halogen atom, and are more preferably methyl groups, ethyl groups, phenyl groups, or naphthyl groups optionally having a halogen atom.

R^{a04} is preferably an alkyl group having 1 to 6 carbon atoms and optionally having a halogen atom or an alicyclic hydrocarbon group having 5 to 12 carbon atoms and optionally having a halogen atom, and is more preferably a methyl group, an ethyl group, a cyclohexyl group or an adamantyl group optionally having a halogen atom.

R^{a6} and R^{a7} are preferably alkyl groups having 1 to 6 carbon atoms and optionally having a halogen atom, alkenyl groups having 2 to 6 carbon atoms and optionally having a halogen atom, or aromatic hydrocarbon groups having 6 to 12 carbon atoms and optionally having a halogen atom, are more preferably methyl groups, ethyl groups, isopropyl groups, t-butyl groups, ethenyl groups, phenyl groups or naphthyl groups optionally having a halogen atom, and are further preferably ethyl groups, isopropyl groups, t-butyl groups, ethenyl groups or phenyl groups optionally having a halogen atom.

m1′ is preferably an integer of 0 to 3, and is more preferably 0 or 1.

n1 is preferably an integer of 0 to 10, is more preferably an integer of 0 to 8, is further preferably an integer of 0 to 3, and is still more preferably 0 or 1.

n1′ is preferably 0 or 1.

Examples of the structural unit (a1-0) include a structural unit represented by any one of formulas (a1-0-1) to (a1-0-24) and a structural unit in which the methyl group corresponding to R^{a01} in the structural unit (a1-0) is replaced with a hydrogen atom, a halogen atom, a haloalkyl group (an alkyl group having a halogen atom) or another alkyl group, and a structural unit represented by any one of formulas (a1-0-1) to (a1-0-10), (a1-0-13), (a1-0-14), and (a1-0-19) to (a1-0-24) is preferable.

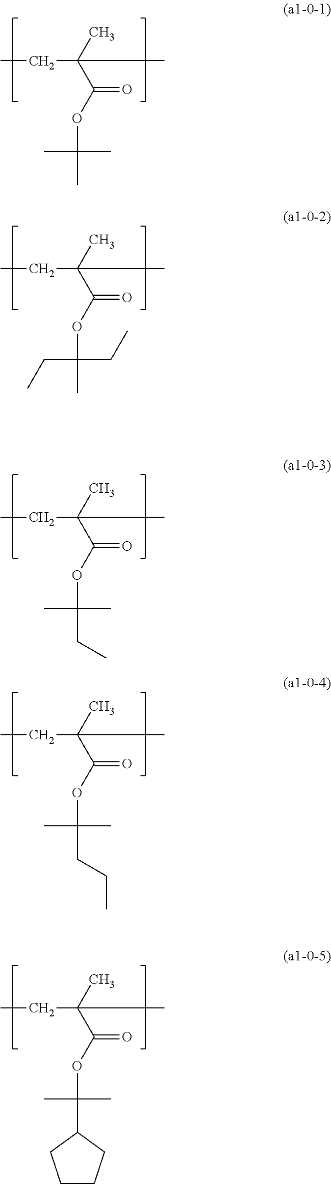

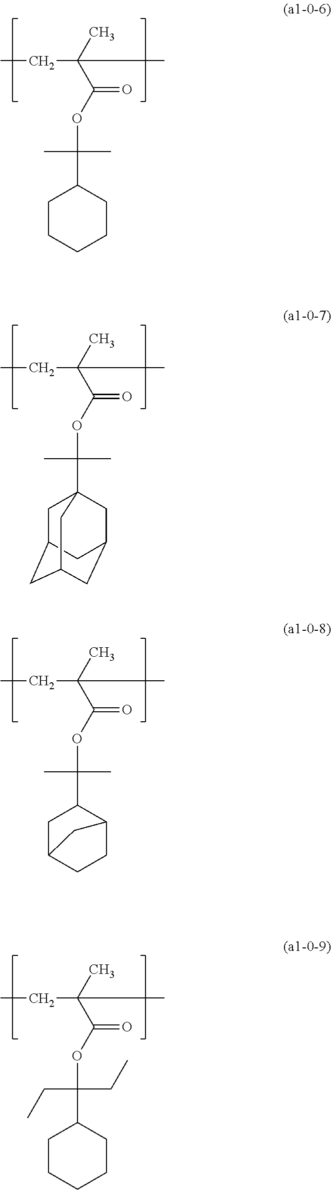

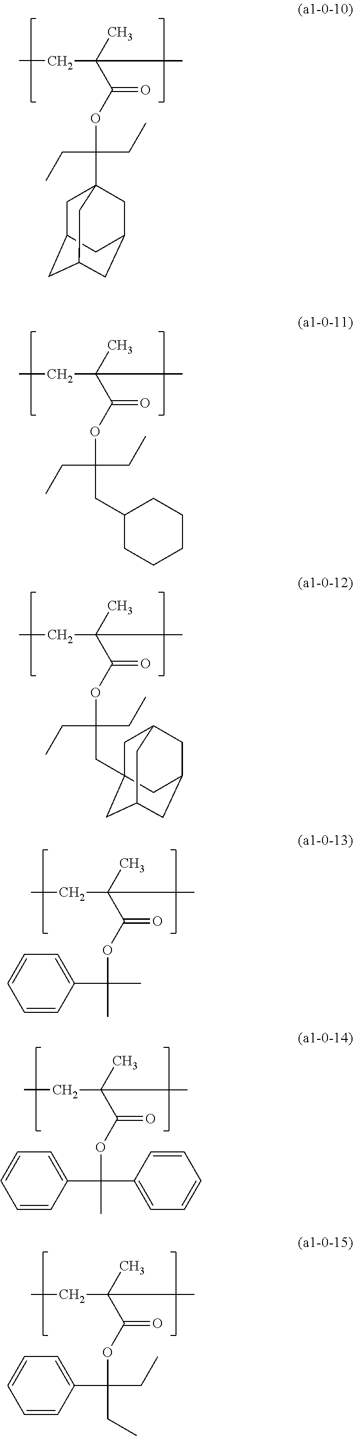

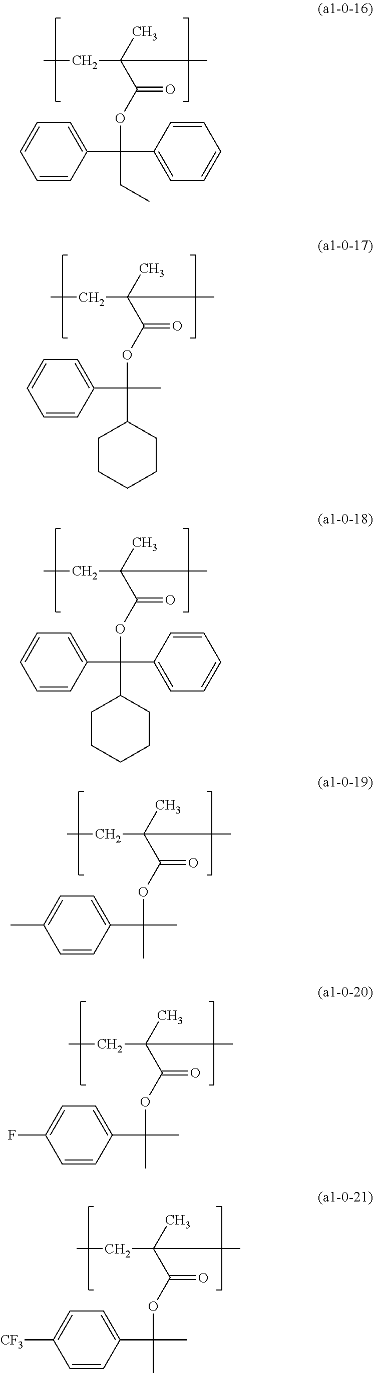

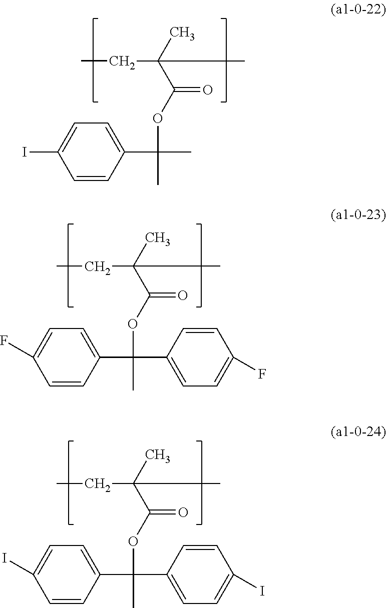

Examples of the structural unit (a1-1) include a structural unit derived from a monomer disclosed in Japanese Patent Laid-open No. 2010-204646. In particular, a structural unit represented by any one of formulas (a1-1-1) to (a1-1-7) and a structural unit in which the methyl group corresponding to Rao in the structural unit (a1-1) is replaced with a hydrogen atom, a halogen atom, a haloalkyl group or another alkyl group are preferable, and a structural unit represented by any one of formulas (a1-1-1) to (a1-1-4) is more preferable.

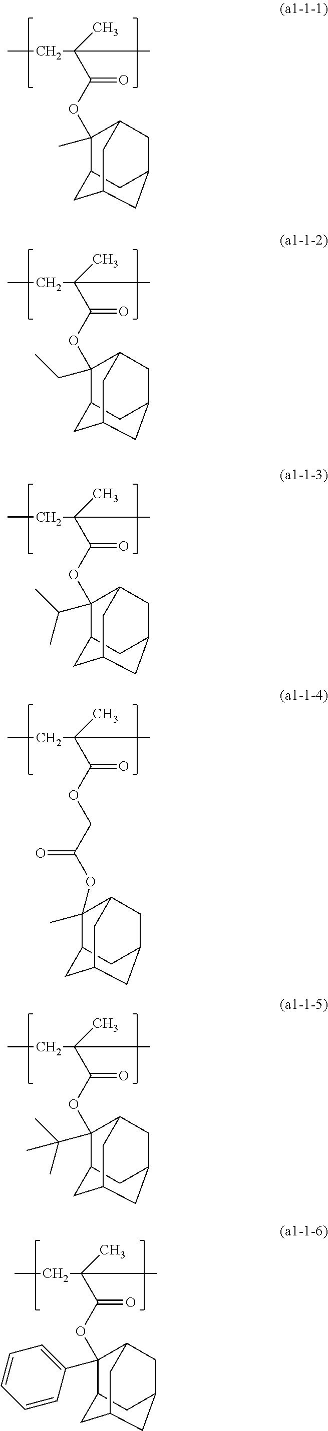

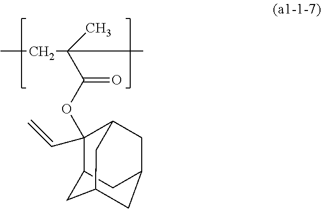

Examples of the structural unit (a1-2) include a structural unit represented by any one of formulas (a1-2-1) to (a1-2-20) or a structural unit in which the methyl group corresponding to Ras in the structural unit (a1-2) is replaced with a hydrogen atom, a halogen atom, a haloalkyl group or another alkyl group, and a structural unit represented by any one of formulas (a1-2-2), (a1-2-5), (a1-2-6), and (a1-2-10) to (a1-2-20) is preferable.

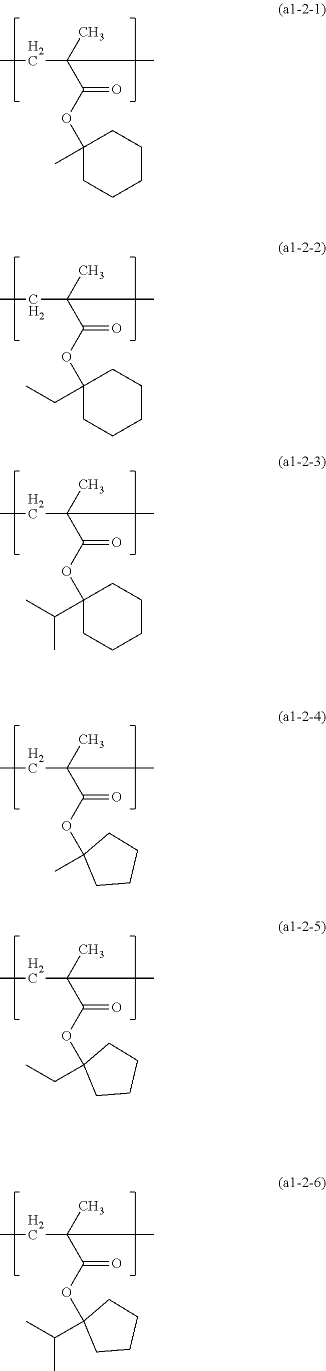

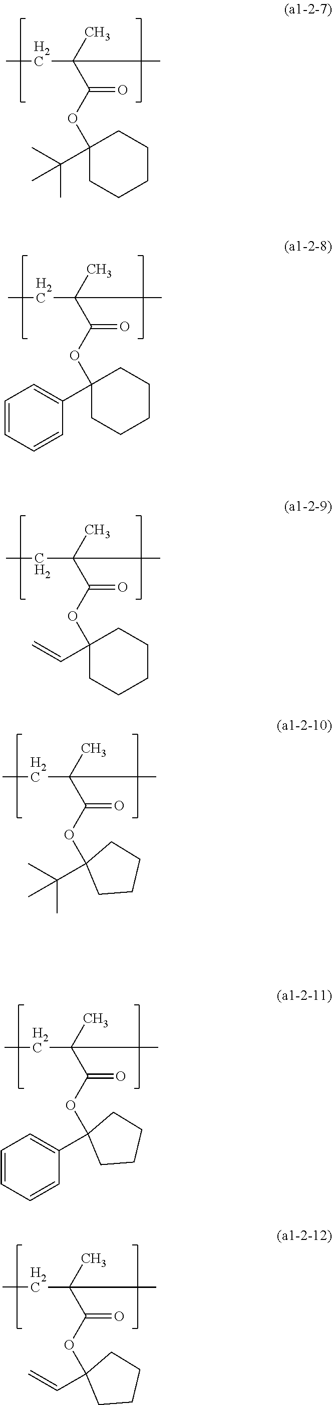

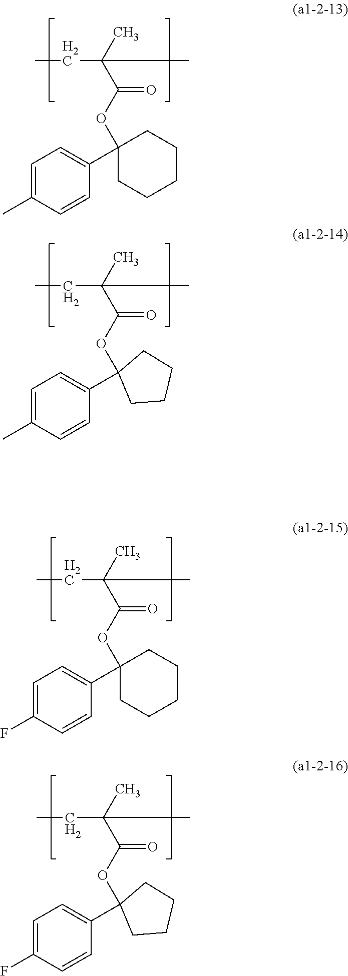

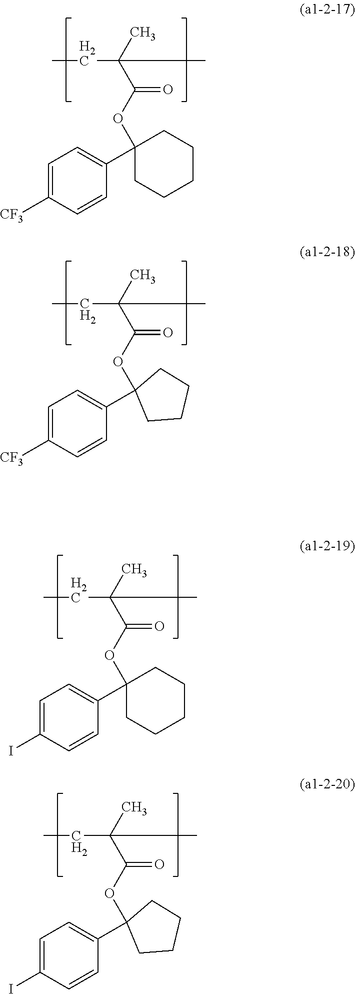

When the resin (A) and/or the like includes the structural unit (a1-0) and/or the structural unit (a 1-1) and/or the structural unit (a 1-2), the total content ratio of these, with respect to all structural units of the resin (A) and/or the like, is, for example, 10 mol % or more, preferably 15 mol % or more, more preferably 20 mol % or more, further preferably 25 mol % or more, still more preferably 30 mol % or more, still further preferably 40 mol % or more, and further more preferably 50 mol % or more. In addition, the total content ratio is, for example, 95 mol % or less, preferably 90 mol % or less, more preferably 85 mol % or less, further preferably 70 mol % or less, and further more preferably 65 mol % or less. Specifically, the total content ratio is, for example, 10 to 95 mol %, preferably 15 to 90 mol %, more preferably 20 to 85 mol %, further preferably 25 to 70 mol %, and further more preferably 30 to 70 mol %.

When the resin (A) and/or the like includes the structural unit (a1-0), the content ratio thereof, with respect to all structural units of the resin (A) and/or the like, is, for example, 5 mol % or more, preferably 10 mol % or more, more preferably 15 mol % or more, further preferably 20 mol % or more, still more preferably 25 mol % or more, still further preferably 30 mol % or more, and further more preferably 35 mol % or more. In addition, the content ratio is, for example, 80 mol % or less, preferably 75 mol % or less, more preferably 70 mol % or less, further preferably 65 mol % or less, and further more preferably 60 mol % or less. Specifically, the content ratio is, for example, 5 to 80 mol %, preferably 5 to 75 mol %, more preferably 10 to 70 mol %, further preferably 10 to 65 mol %, and further more preferably 10 to 60 mol %.

When the resin (A) and/or the like includes the structural unit (a1-1) and/or the structural unit (a1-2), the total content ratio of these, with respect to all structural units of the resin (A) and/or the like, is, for example, 10 mol % or more, preferably 15 mol % or more, more preferably 20 mol % or more, further preferably 25 mol % or more, still more preferably 30 mol % or more, still further preferably 40 mol % or more, and further more preferably 50 mol % or more. In addition, the total content ratio is, for example, 95 mol % or less, preferably 90 mol % or less, more preferably 85 mol % or less, further preferably 80 mol % or less, still more preferably 75 mol % or less, still further preferably 70 mol % or less, and further more preferably 65 mol % or less. Specifically, the total content ratio is, for example, 10 to 90 mol %, preferably 15 to 85 mol %, more preferably 15 to 80 mol %, further preferably 20 to 80 mol %, still more preferably 20 to 75 mol %, still further preferably 20 to 70 mol %, even more preferably 20 to 65 mol %, and further more preferably 20 to 60 mol %.

Examples of the structural unit (a1) having the group (2) include a structural unit represented by formula (a1-4) (hereinafter referred to as “structural unit (a1-4)” in some cases):

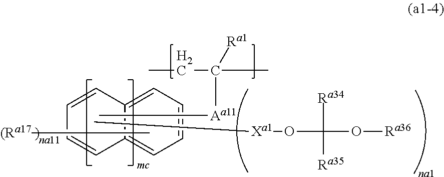

wherein all symbols in formula (a1-4) have the same meanings as described above.

Examples of the halogen atom and the alkyl group optionally having a halogen atom in R^{a1} and R^{a17} include the same as those exemplified for R^{a01}, R^{a4} and R^{a5} in formulas (a1-0) to (a1-2).

R^{a1} is preferably a hydrogen atom or an alkyl group having 1 to 4 carbon atoms, is more preferably a hydrogen atom, a methyl group or an ethyl group, and is further preferably a hydrogen atom or a methyl group.

Examples of the alkoxy group, the alkoxyalkyl group, the alkoxyalkoxy group, the alkylcarbonyl group, and the alkylcarbonyloxy group in R^{a17} include the same groups as those exemplified in the present specification, within the range permitted by the upper limit of the number of carbon atoms. The number of carbon atoms of the alkoxy group is preferably 1 to 4, and is more preferably 1 to 3. The alkoxy group is preferably an alkoxy group having 1 to 4 carbon atoms, is more preferably an alkoxy group having 1 to 3 carbon atoms, is further preferably a methoxy group or an ethoxy group, and is further more preferably a methoxy group. The number of carbon atoms of the alkoxyalkyl group is preferably 2 to 8, and is more preferably 2 to 4. The alkoxyalkyl group is preferably an alkoxyalkyl group having 2 to 8 carbon atoms, is more preferably an alkoxyalkyl group having 2 to 4 carbon atoms, is further preferably a methoxymethyl group or an ethoxyethyl group, and is further more preferably a methoxymethyl group. The number of carbon atoms of the alkoxyalkoxy group is preferably 2 to 8, and is more preferably 2 to 4. The alkoxyalkoxy group is preferably an alkoxyalkoxy group having 2 to 8 carbon atoms, is more preferably an alkoxyalkoxy group having 2 to 4 carbon atoms, and is further preferably a methoxyethoxy group or an ethoxyethoxy group. The alkylcarbonyl group is preferably an alkylcarbonyl group having 2 to 4 carbon atoms, is more preferably an alkylcarbonyl group having 2 to 3 carbon atoms, and is further preferably an acetyl group. The alkylcarbonyloxy group is preferably an alkylcarbonyloxy group having 2 to 4 carbon atoms, is more preferably an alkylcarbonyloxy group having 2 to 3 carbon atoms, and is further preferably an acetyloxy group.

R^{a17} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, is more preferably a fluorine atom, an iodine atom, a hydroxy group, a methyl group, a methoxy group, an ethoxy group, an ethoxyethoxy group or an ethoxymethoxy group, and is further preferably a fluorine atom, an iodine atom, a hydroxy group, a methyl group, a methoxy group or an ethoxyethoxy group.

Examples of the alkanediyl group of A^{a11} include: a straight-chain alkanediyl group such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and dodecane-1,12-diyl group; and a branched alkanediyl group such as a butane-1,3-diyl group, a 2-methylpropane-1,3-diyl group, a 2-methylpropane-1,2-diyl group, a pentane-1,4-diyl group, a 2-methylbutane-1,4-diyl group, a heptane-1,6-diyl group, an octane-1,7-diyl group, a nonane-1,8-diyl group, a decane-1,9-diyl group and an undecane-1,10-diyl group. The number of carbon atoms of the alkanediyl group is preferably 1 to 10, is more preferably 1 to 8, is further preferably 1 to 6, is still more preferably 1 to 4, is still further preferably 1 to 3, and is further more preferably 1 or 2.

Examples of the alkyl group of R^{a18} include a methyl group, an ethyl group, a n-propyl group, an isopropyl group, a n-butyl group and a tert-butyl group.

When —CH₂— contained in the alkanediyl group of A^{a11} is replaced with —O—, —CO— or —NR^{a18}—, the number of carbon atoms before the replacement is regarded as the number of carbon atoms of the alkanediyl group.

Examples of the group formed by replacing —CH₂— contained in the alkanediyl group of A^{a11} with —O—, —CO— or —NR^{a18} include a hydroxy group, a carboxy group, a carbonyl group, an oxy group, an amino group, an alkoxy group, an alkoxycarbonyl group, an alkylcarbonyl group, an alkylamino group, a peptide group, an alkanediyloxy group, an alkanediyloxycarbonyl group, an alkanediylcarbonyl group, an alkanediylcarbonyloxy group, an alkanediylsulfonyl group, an alkanediylthio group, and an alkanediylamino group. Examples of these replaced groups include the same as those exemplified in the present specification, within the range permitted by the upper limit of the number of carbon atoms.

For example, examples of the group formed by replacing —CH₂-contained in the alkanediyl group of A^{a11} with —O—, —CO— or —NR^{a18} include *—O—, *—CO—O—, *—O—CO—, *—CO—O-A^{a12}-CO—O—, *—O—CO-A^{a12}-O—, *—O-A^{a12}-CO—O—, *—CO—O-A^{a12}-O—CO—, *—O—CO-A^{a12}-O—CO— and *—CO—NR^{a18}—. In particular, *—CO—O—, *—CO—O-A^{a12}-CO—O— or *—O-A^{a12}-CO—O— and *—CO—NR^{a18}— are preferable. Here, A^{a12} represents an alkanediyl group having 1 to 8 carbon atoms, and * represents a bonding site to a carbon atom to which R^{a1} is bonded. Examples of the alkanediyl group of A^{a12} include the same alkanediyl group as A^{a11} within the range permitted by the upper limit of the number of carbon atoms.

A^{a11} is preferably a single bond, *—CO—O— or *—CO—O-A^{a12}-CO—O—, is more preferably a single bond, *—CO—O— or *—CO—O—CH₂—CO—O—, and is further preferably a single bond or *—CO—O—.

na1 is preferably 1, 2, 3 or 4, is more preferably 1, 2 or 3, and is further preferably 1 or 2.

na11 is preferably 0, 1, 2 or 3, is more preferably 0, 1 or 2, and is further preferably 0 or 1.

mc is preferably 0 or 1.

Examples of R^{a34}, R^{a35} and R^{a36} include the same as those exemplified for R^{a1′}, R^{a2′} and R^{a3′} in formula (2).

When R^{a35} and R^{a36} are bonded to each other to form a divalent heterocyclic group having 3 to 20 carbon atoms together with —C—O— to which they are bonded, examples thereof include the following groups. * represents a bonding site, and one of them is a bonding site to R^{a34}.

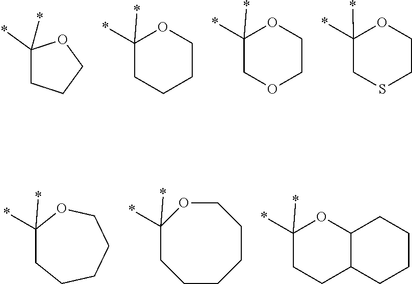

R^{a34} is preferably a hydrogen atom.

R^{a35} is preferably a hydrogen atom, an alkyl group having 1 to 12 carbon atoms or an alicyclic hydrocarbon group having 3 to 12 carbon atoms, and is more preferably a methyl group or an ethyl group.

The hydrocarbon group of R^{a36} is preferably an alkyl group having 1 to 18 carbon atoms, an alicyclic hydrocarbon group having 3 to 18 carbon atoms, an aromatic hydrocarbon group having 6 to 18 carbon atoms or a group formed by combining these groups, and is more preferably an alkyl group having 1 to 18 carbon atoms, an alicyclic hydrocarbon group having 3 to 18 carbon atoms or an aralkyl group having 7 to 18 carbon atoms. The alkyl group and the alicyclic hydrocarbon group in R^{a36} are preferably unsubstituted. The aromatic hydrocarbon group in R^{a36} is preferably an aromatic ring having an aryloxy group having 6 to 10 carbon atoms.

—O—C(R^{a34})(R^{a35})—O—R^{a36} in the structural unit (a1-4) is eliminated upon contact with an acid (for example, p-toluenesulfonic acid) to form a hydroxy group or a carboxy group.

In the case of a benzene ring, —X^{a1}—O—C(R^{a34})(R^{a35})—O—R^{a36} may be bonded to any of the o-position, m-position or p-position with respect to the bonding position of A^{a11}. In particular, at least one is preferably bonded to the o-position or p-position, and more preferably bonded to the p-position. In the case of a naphthalene ring, when A^{a11} is bonded to the 1st position, the above group may be bonded to any of the 2nd to 8th positions, and when A^{a11} is bonded to the 2nd position, the above group may be bonded to any of the 1st and 3rd to 8th positions. In particular, when A^{a11} is bonded to the 1st position, at least one is preferably bonded to the 3rd to 6th positions, and more preferably bonded to the 3rd or 4th position. When A^{a11} is bonded to the 2nd position, the above group is preferably bonded to the 4th to 7th positions, and more preferably bonded to the 5th or 6th position.

Examples of the structural unit (a1-4) include a structural unit derived from a monomer disclosed in Japanese Patent Laid-Open No. 2010-204646. Preferable are the respective structural units represented by formulas (a1-4-1) to (a1-4-42) and structural units in which the hydrogen atom corresponding to R^{a1} in the structural unit (a1-4) is replaced with a halogen atom, a haloalkyl group or an alkyl group, and more preferable are the respective structural units represented by formulas (a1-4-1) to (a1-4-5), (a1-4-10), (a1-4-13), (a1-4-14), (a1-4-19) and (a1-4-20).

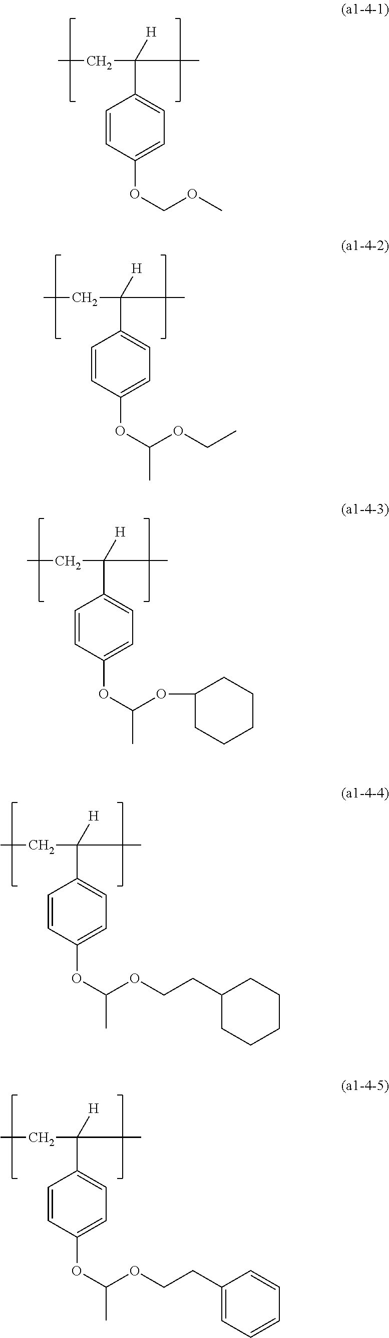

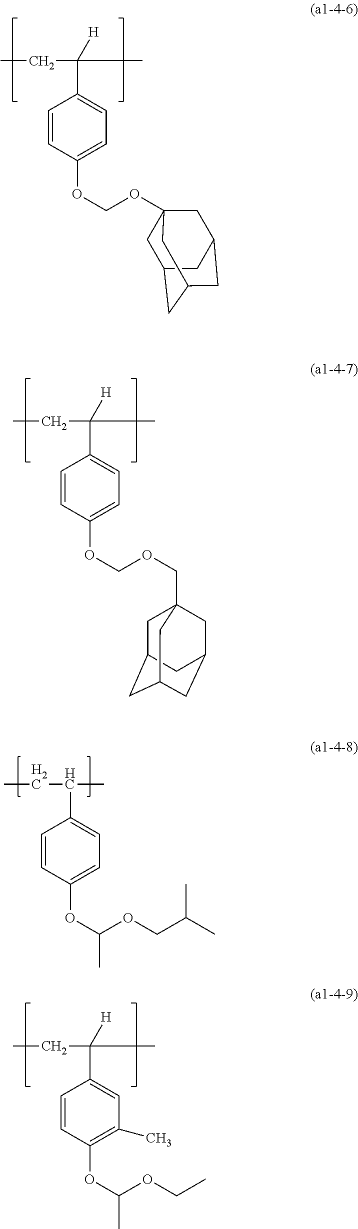

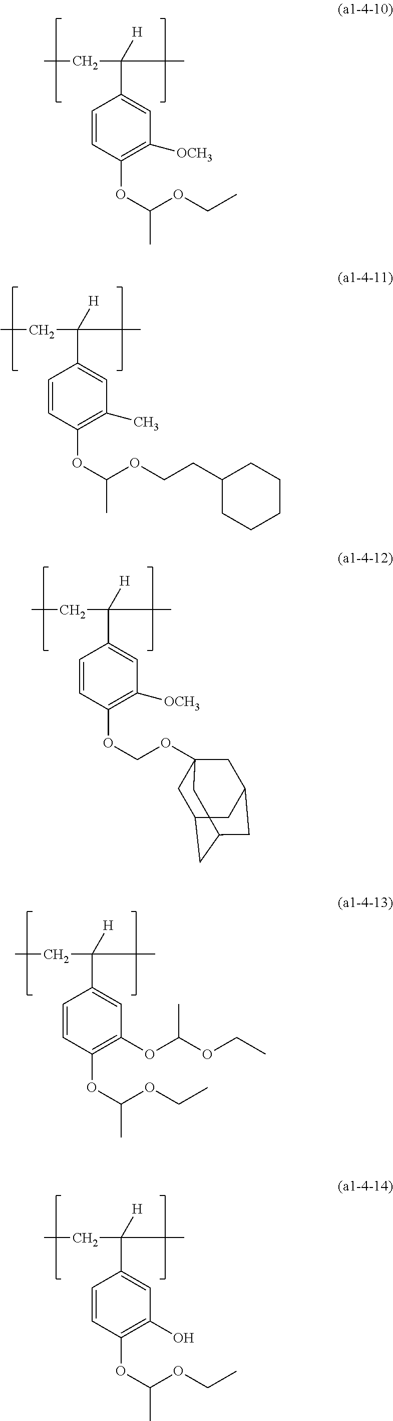

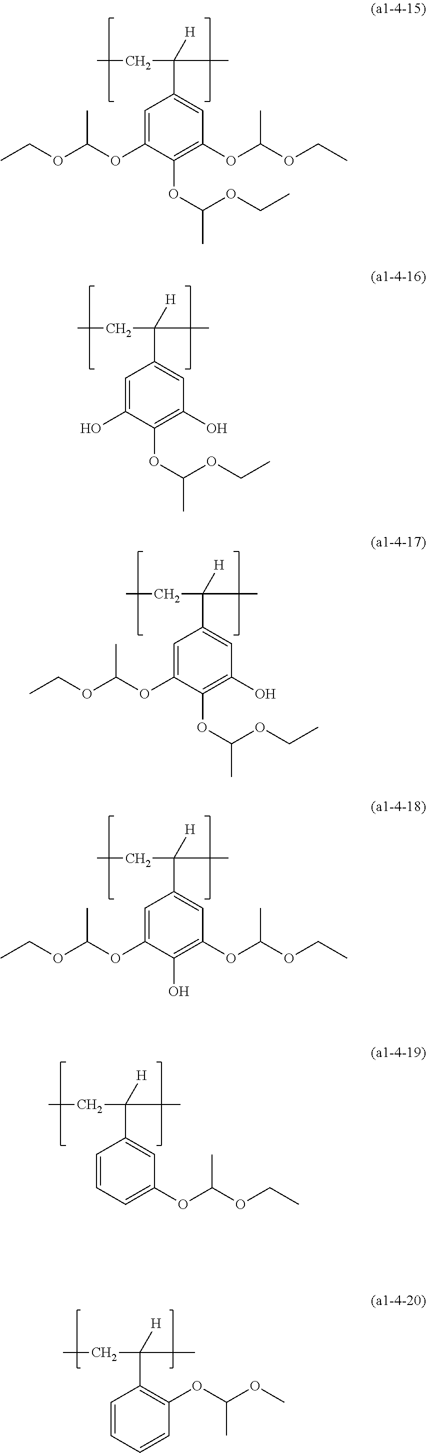

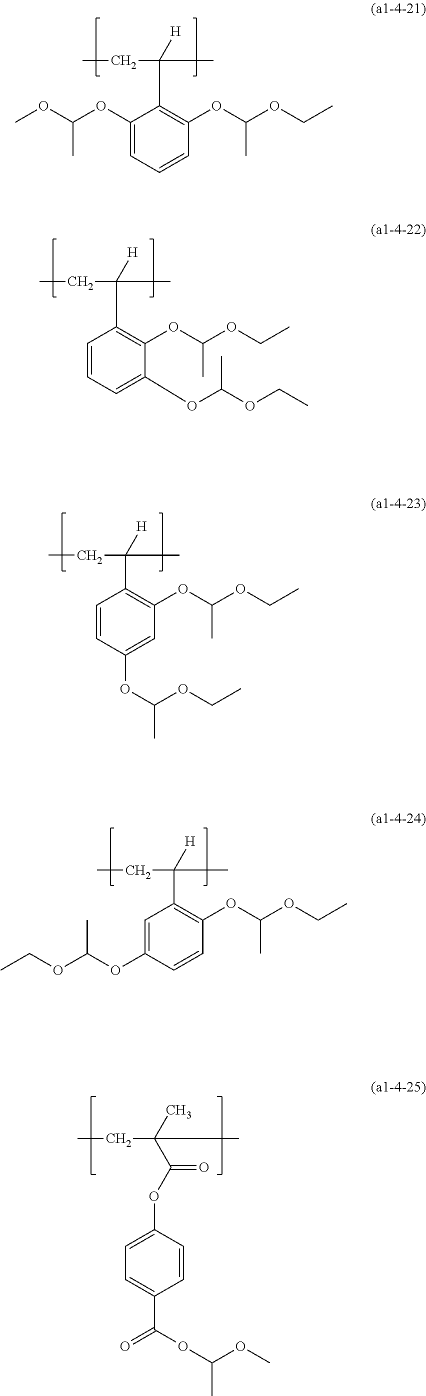

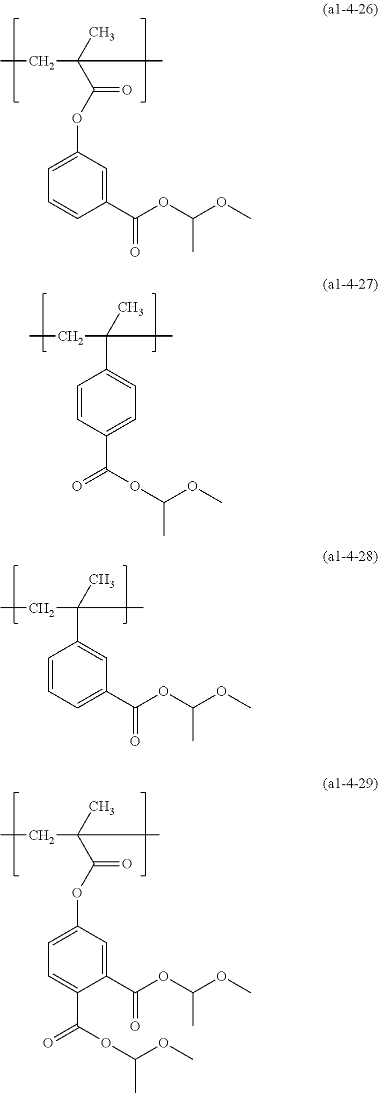

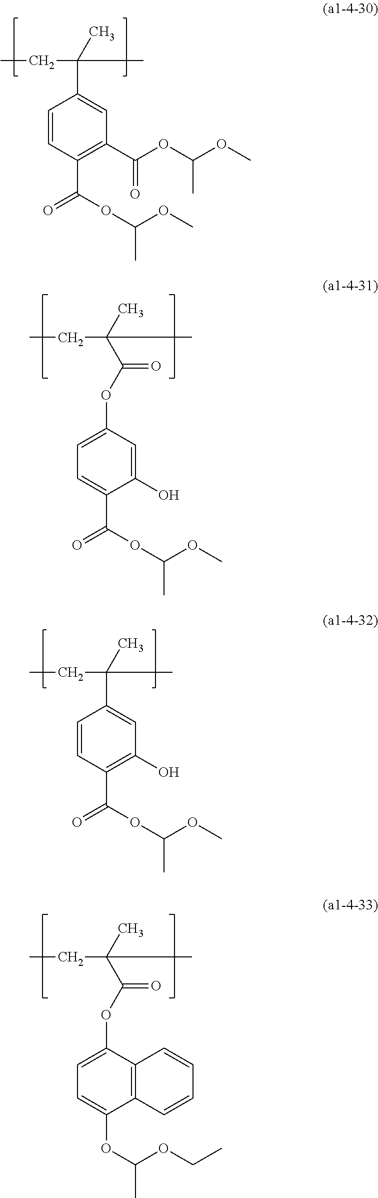

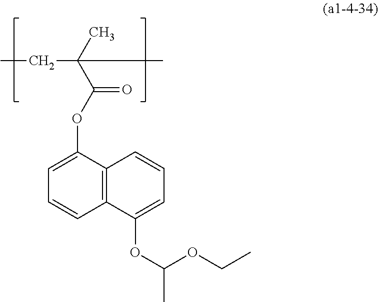

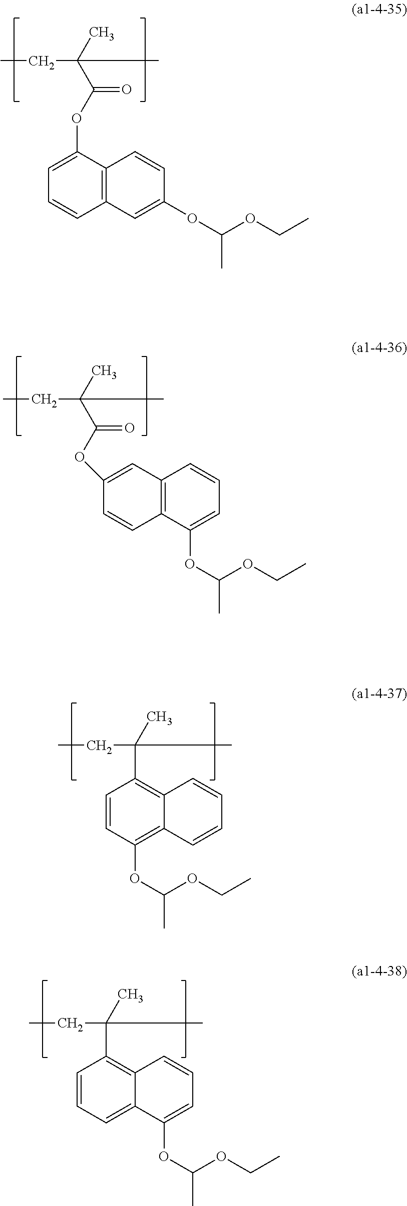

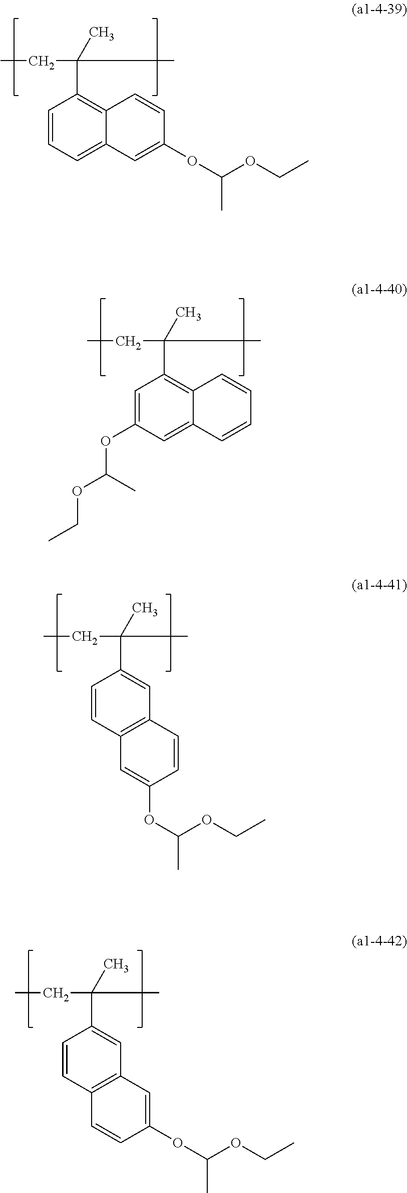

When the resin (A) and/or the like includes the structural unit (a1-4), the content ratio thereof, with respect to the total of all structural units of the resin (A) and/or the like, is, for example, 10 mol % or more, preferably 15 mol % or more, more preferably 20 mol % or more, further preferably 25 mol % or more, still more preferably 30 mol % or more, still further preferably 40 mol % or more, and further more preferably 50 mol % or more. In addition, the content ratio is, for example, 95 mol % or less, preferably 90 mol % or less, more preferably 85 mol % or less, further preferably 80 mol % or less, still more preferably 75 mol % or less, still further preferably 70 mol % or less, and further more preferably 65 mol % or less. Specifically, the content ratio is preferably 10 to 95 mol %, more preferably 15 to 90 mol %, further preferably 20 to 85 mol %, further more preferably 20 to 70 mol %, and particularly preferably 20 to 60 mol %.

Examples of the structural unit derived from a (meth)acrylic monomer having the group (2) also include a structural unit represented by formula (a1-5) (hereinafter referred to as “structural unit (a1-5)” in some cases):

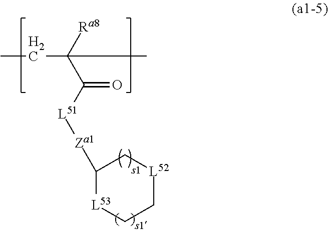

wherein all symbols in formula (a1-5) have the same meanings as described above.

Examples of the halogen atom and the alkyl group optionally having a halogen atom in R^{a8} of formula (a1-5) include the same as those exemplified for R^{a01}, R^{a4} and R^{a5} in formulas (a1-0) to (a1-2).

In formula (a1-5), R^{a8} is preferably a hydrogen atom, a methyl group or a trifluoromethyl group.

L⁵¹ is preferably an oxygen atom.

It is preferable that one of L⁵² and L⁵³ is —O— and the other is —S—.

s1 is preferably 1.

s1′ is preferably an integer of 0 to 2.

Z^{a1} is preferably a single bond or *—CH₂—CO—O—.

Examples of the structural unit (a1-5) include a structural unit derived from a monomer disclosed in Japanese Patent Laid-Open No. 2010-61117. In particular, the respective structural units represented by formulas (a1-5-1) to (a1-5-4) are preferable, and the structural unit represented by formula (a1-5-1) or (a1-5-2) is more preferable.

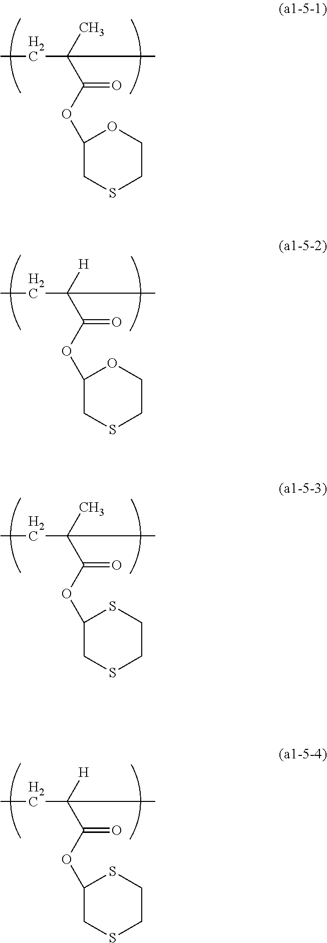

When the resin (A) and/or the like includes the structural unit (a1-5), the content ratio thereof, with respect to all structural units of the resin (A) and/or the like, is, for example, 1 mol % or more, preferably 2 mol % or more, more preferably 3 mol % or more, further preferably 5 mol % or more, still more preferably 10 mol % or more, still further preferably 20 mol % or more, and further more preferably 25 mol % or more. In addition, the content ratio is, for example, 80 mol % or less, preferably 70 mol % or less, more preferably 60 mol % or less, further preferably 50 mol % or less, still more preferably 45 mol % or less, still further preferably 40 mol % or less, and further more preferably 30 mol % or less. Specifically, the content ratio is preferably 1 to 50 mol %, more preferably 3 to 45 mol %, further preferably 5 to 40 mol %, and further more preferably 5 to 30 mol %.

Examples of the structural unit (a1) having the group (1) include a structural unit represented by formula (a1-6) (hereinafter referred to as “structural unit (a1-6)” in some cases):

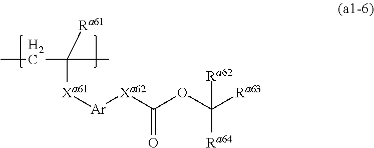

wherein all symbols in formula (a1-6) have the same meanings as described above.

Examples of the halogen atom and the alkyl group optionally having a halogen atom in R^{a61} of formula (a1-6) include the same as those exemplified for R^{a01}, R^{a4} and R^{a5} in formulas (a1-0) to (a1-2).

R^{a61} is preferably a hydrogen atom, a methyl group or a trifluoromethyl group.

Examples of the alkyl group in R^{a62}, R^{a63}, R^{a64} and R^{a65} include a methyl group, an ethyl group, a propyl group, a butyl group, a pentyl group and a hexyl group. The alkyl group is preferably an alkyl group having 1 to 4 carbon atoms, is more preferably an alkyl group having 1 to 3 carbon atoms, and is further preferably a methyl group or an ethyl group.

Examples of the cyclic hydrocarbon group in R^{a62}, R^{a63} and R^{a64} include an alicyclic hydrocarbon group and an aromatic hydrocarbon group.

The alicyclic hydrocarbon group may be either monocyclic or polycyclic. Examples of the monocyclic alicyclic hydrocarbon group include a cyclopentyl group, a cyclohexyl group, a cycloheptyl group and a cyclooctyl group. Examples of the polycyclic alicyclic hydrocarbon group include a decahydronaphthyl group, an adamantyl group and a norbornyl group. The number of carbon atoms of the alicyclic hydrocarbon group is preferably 3 to 16, and is more preferably 3 to 12.

Examples of the aromatic hydrocarbon group include a phenylene group and a naphthylene group.

Examples of the substituent that the cyclic hydrocarbon group optionally has include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group and a methacryloyloxy group. Examples of these groups include the same as those exemplified for Rail in formula (a1-4).

When R^{a62} and R^{a63} are bonded to each other to form a ring, examples thereof include an adamantane ring, a cyclopentane ring and a cyclohexane ring. Specifically, when R^{a62} and R^{a63} are bonded to each other to form a ring, examples of —C(R^{a62})(R^{a63})(R^{a64}) include the following groups. * represents a bonding site to an oxygen atom. The number of carbon atoms of the ring is preferably 3 to 16, and is more preferably 3 to 12.

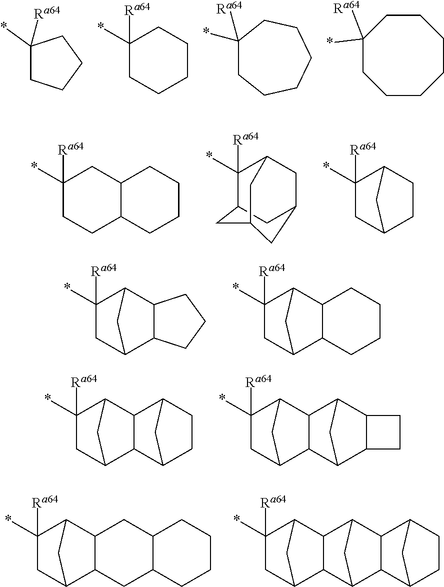

Examples of the alkanediyl group having 1 to 4 carbon atoms in L^{a61} and L^{a62} include a methylene group, an ethylene group, a propane-1,3-diyl group, a propane-1,2-diyl group, a butane-1,4-diyl group, a butane-1,3-diyl group, a 2-methylpropane-1,3-diyl group and a 2-methylpropane-1,2-diyl group.

It is preferable that L^{a61} and L^{a62} are each independently a methylene group or an ethylene group.

X^{a61} is preferably a single bond or —CO—O—*, and is more preferably a single bond.

X^{a62} is preferably a single bond or *—O-L^{a61}-, and is more preferably a single bond.

Examples of the aromatic hydrocarbon group having 6 to 20 carbon atoms of Ar include a phenylene group, a 1-naphthylene group, a 2-naphthylene group, a 1-anthrylene group, a 9-anthrylene group, a biphenylene group, a 1-phenanthrylene group and a 2-phenanthrylene group.

Examples of the substituent that the aromatic hydrocarbon group optionally has include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group and a methacryloyloxy group. Examples of these groups include the same as those exemplified for Rail in formula (a1-4).

The alkyl group is preferably an alkyl group having 1 to 4 carbon atoms, is more preferably a methyl group or an ethyl group, and is further preferably a methyl group. The alkoxy group is preferably an alkoxy group having 1 to 4 carbon atoms, is more preferably a methoxy group or an ethoxy group, and is further preferably a methoxy group. The alkoxyalkyl group is preferably an alkoxyalkyl group having 2 to 8 carbon atoms, is more preferably a methoxymethyl group or an ethoxyethyl group, and is further preferably a methoxymethyl group. The alkoxyalkoxy group is preferably an alkoxyalkoxy group having 2 to 8 carbon atoms, and is more preferably a methoxyethoxy group or an ethoxyethoxy group. The alkylcarbonyl group is preferably an alkylcarbonyl group having 2 to 3 carbon atoms, and is more preferably an acetyl group. The alkylcarbonyloxy group is preferably an alkylcarbonyloxy group having 2 to 3 carbon atoms, and is more preferably an acetyloxy group.

The substituent is preferably a halogen atom, a hydroxy group, an alkyl group having 1 to 4 carbon atoms, an alkoxy group having 1 to 4 carbon atoms or an alkoxyalkoxy group having 2 to 8 carbon atoms, is more preferably a fluorine atom, an iodine atom, a hydroxy group, a methyl group, a methoxy group, an ethoxy group, an ethoxyethoxy group or an ethoxymethoxy group, and is further preferably a fluorine atom, an iodine atom, a hydroxy group, a methyl group, a methoxy group or an ethoxyethoxy group.

Ar is preferably a phenylene group optionally having a substituent, and is more preferably a phenylene group optionally having a hydroxy group.

Examples of the structural unit (a1-6) include the respective structural units represented by formulas (a1-6-1) to (a1-6-44), and the respective structural units represented by formulas (a1-6-1) to (a1-6-9) and (a1-6-37) to (a1-6-44) are preferable, the respective structural units represented by formulas (a1-6-1), (a1-6-2), (a1-6-4), (a1-6-5), (a1-6-7), (a1-6-8), and (a1-6-37) to (a1-6-44) are more preferable, and the respective structural units represented by formulas (a1-6-1), (a1-6-2), (a1-6-7), (a1-6-8), and (a1-6-37) to (a1-6-44) are further preferable. Examples of the structural unit (a1-6) also include structural units in which the hydrogen atom corresponding to R^{a61} in the following structural units is replaced with a methyl group or the like, and structural units in which the methyl group corresponding to R^{a61} in the following structural units is replaced with a hydrogen atom or the like.

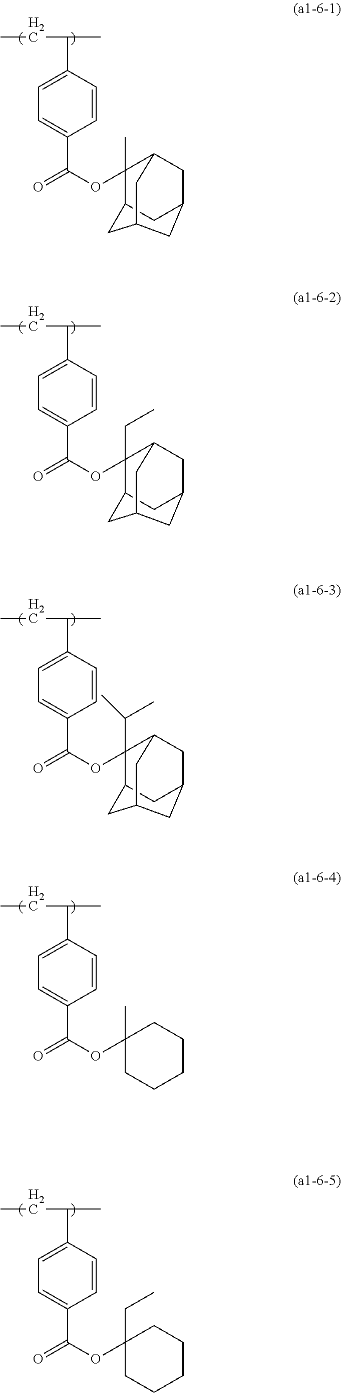

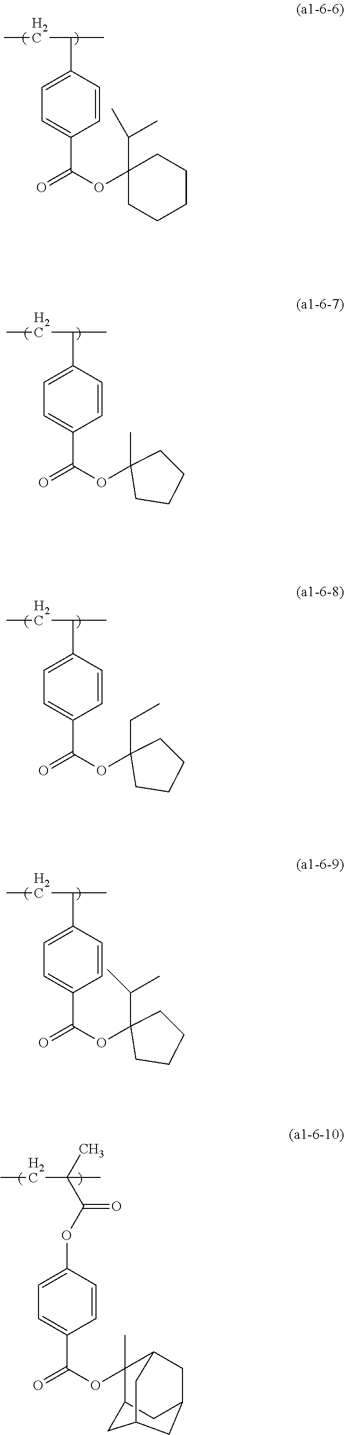

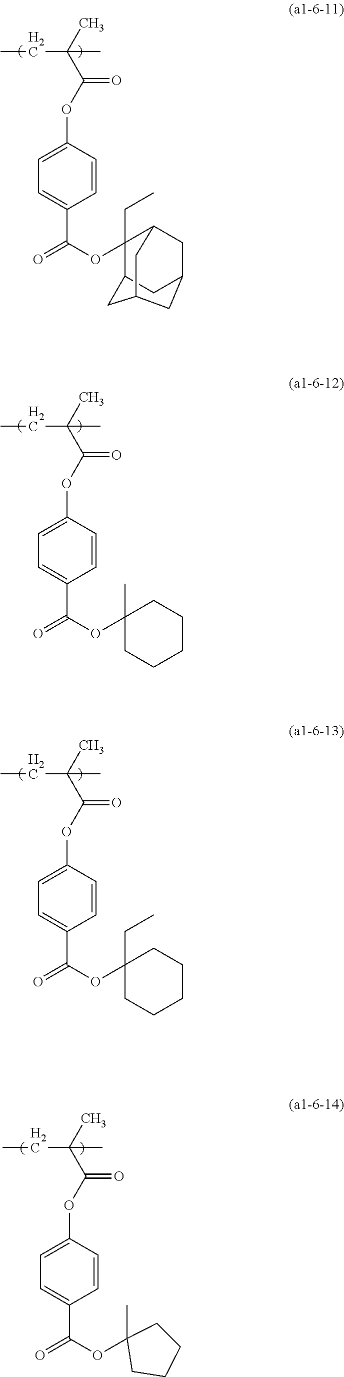

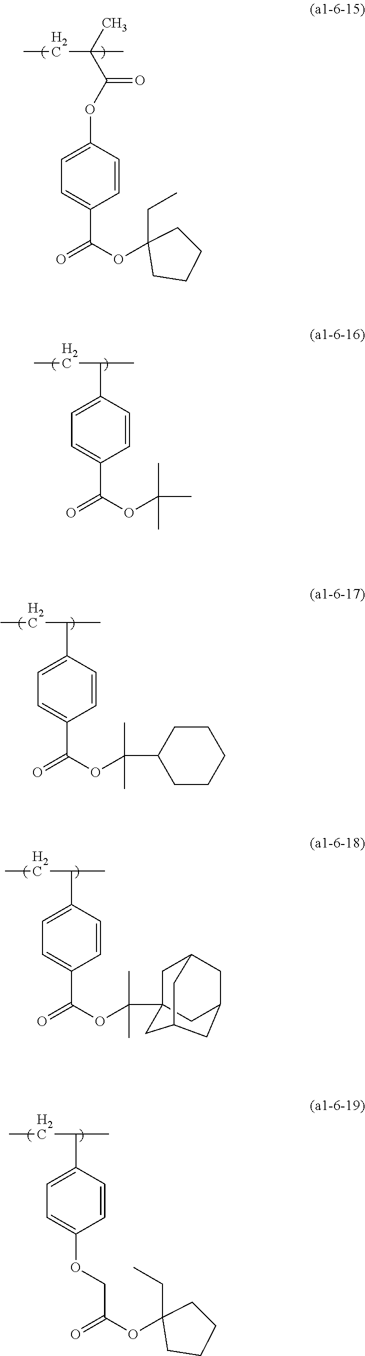

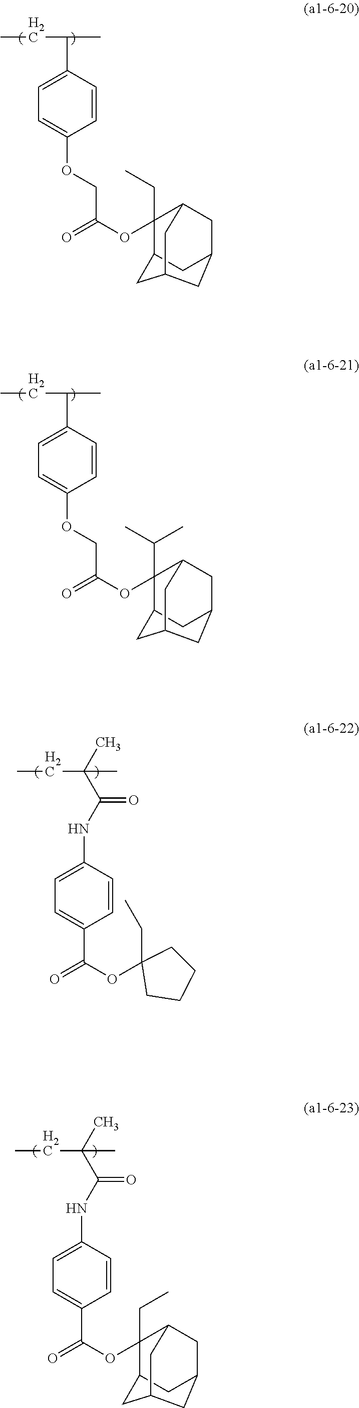

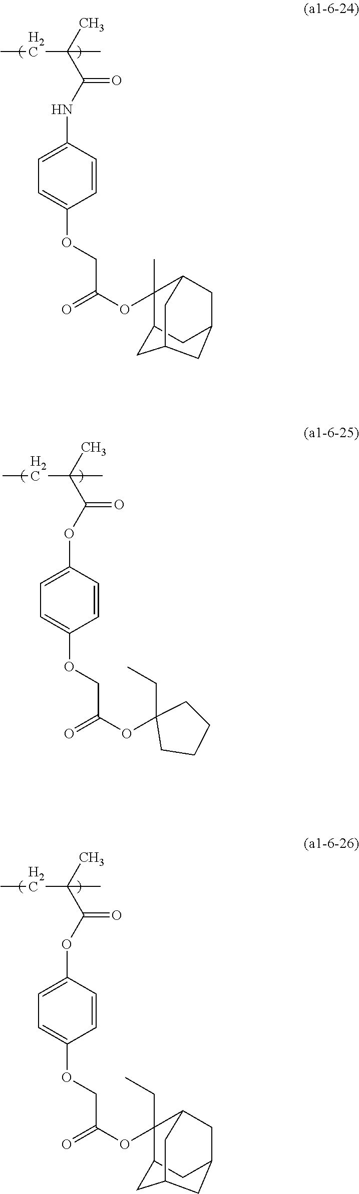

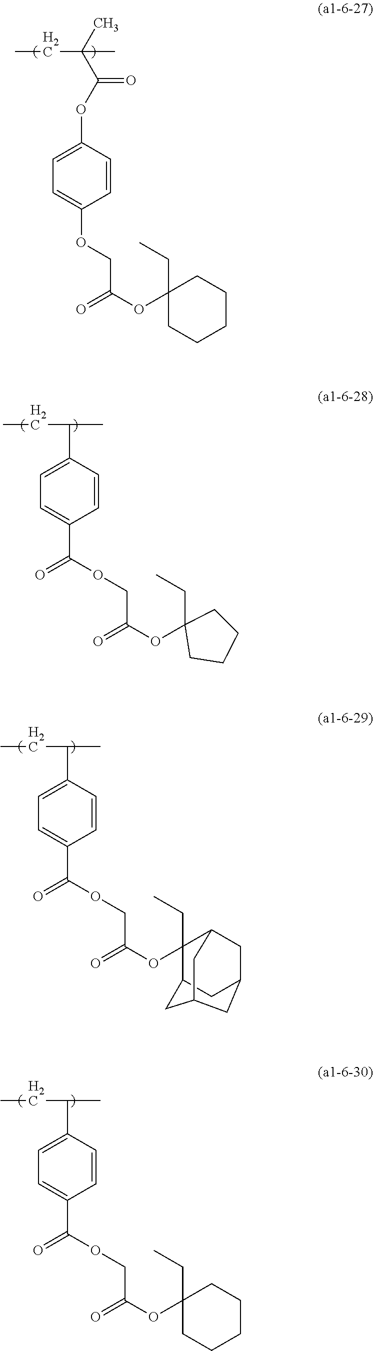

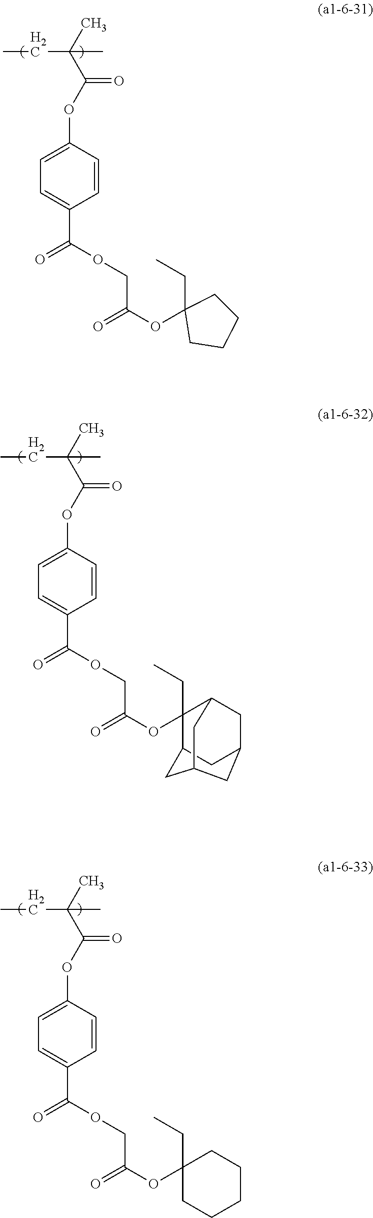

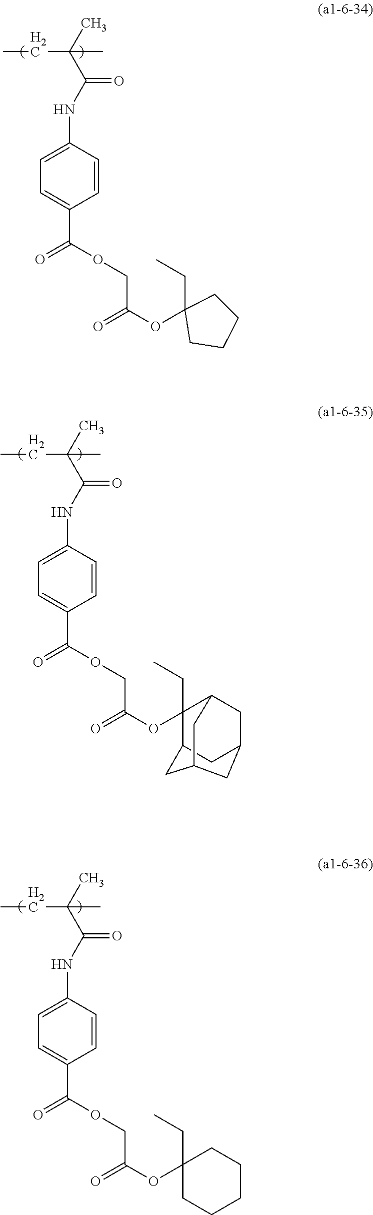

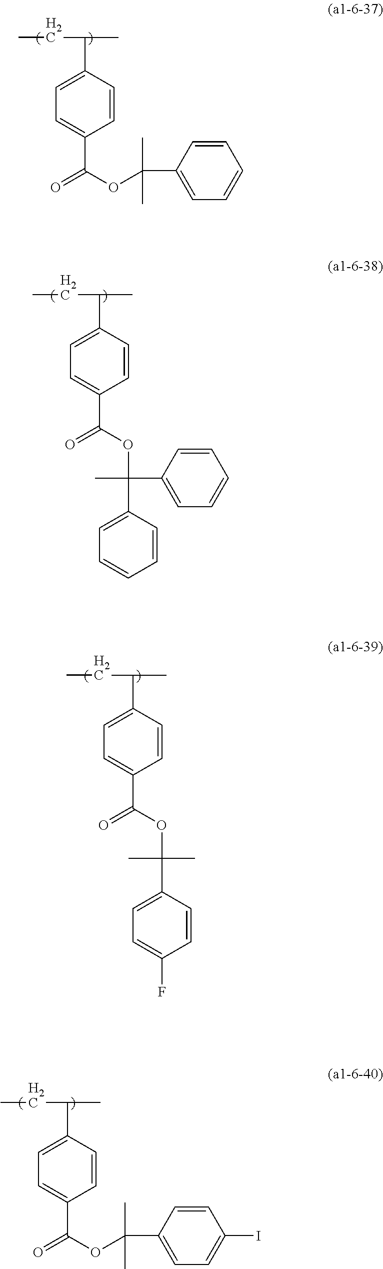

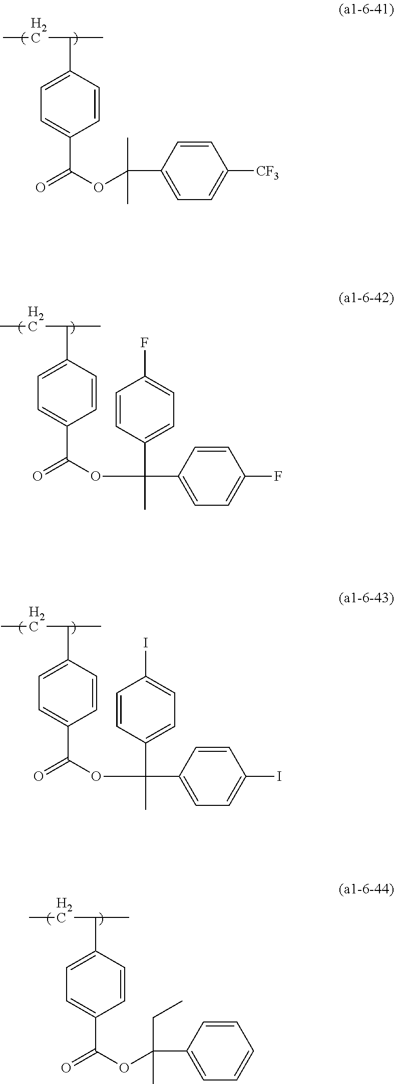

When the resin (A) and/or the like includes the structural unit (a1-6), the content ratio thereof, with respect to all structural units of the resin (A) and/or the like, is, for example, 3 mol % or more, preferably 5 mol % or more, more preferably 7 mol % or more, further preferably 10 mol % or more, still more preferably 20 mol % or more, still further preferably 30 mol % or more, and further more preferably 40 mol % or more. In addition, the content ratio is, for example, 80 mol % or less, preferably 75 mol % or less, more preferably 70 mol % or less, and further preferably 65 mol % or less. Specifically, the content ratio is preferably 3 to 80 mol %, more preferably 5 to 75 mol %, further preferably 7 to 70 mol %, further more preferably 7 to 65 mol %, and particularly preferably 10 to 60 mol %.

Examples of the structural unit (a1) also include the following structural units.

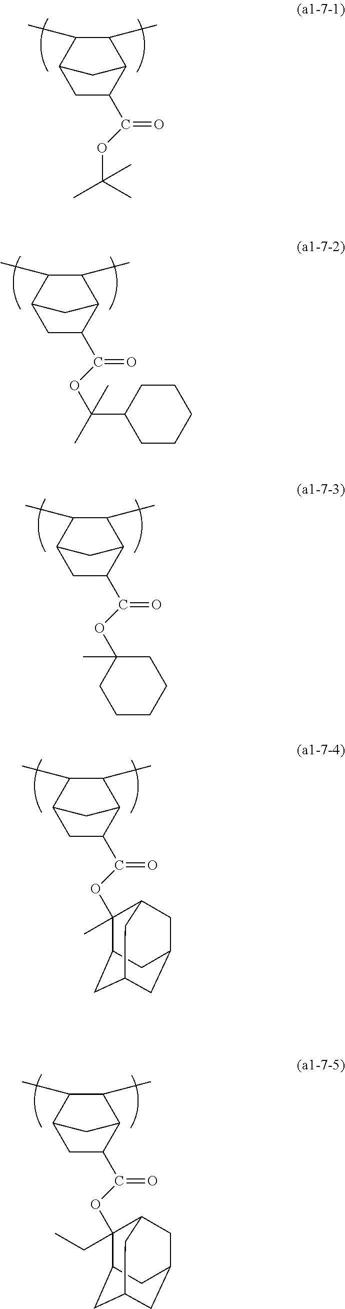

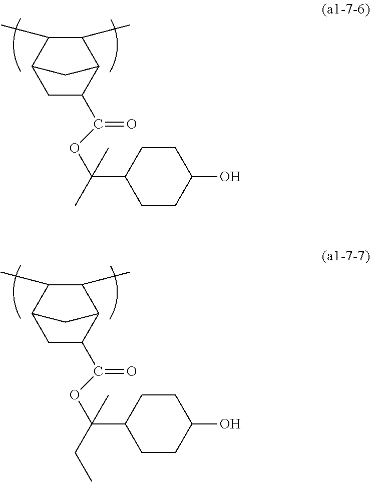

When the resin (A) and/or the like includes the structural units represented by formulas (a1-7-1) to (a1-7-7), the content ratio thereof, with respect to all structural units of the resin (A) and/or the like, is, for example, 10 mol % or more, preferably 15 mol % or more, more preferably 20 mol % or more, further preferably 25 mol % or more, still more preferably 30 mol % or more, still further preferably 40 mol % or more, and further more preferably 50 mol % or more. In addition, the content ratio is, for example, 95 mol % or less, preferably 90 mol % or less, more preferably 85 mol % or less, further preferably 80 mol % or less, still more preferably 75 mol % or less, still further preferably 70 mol % or less, and further more preferably 60 mol % or less. Specifically, the content ratio is preferably 10 to 95 mol %, more preferably 15 to 90 mol %, further preferably 20 to 85 mol %, further more preferably 20 to 70 mol %, and particularly preferably 20 to 60 mol %.

Examples of the structural unit (a1) also include the following structural units.

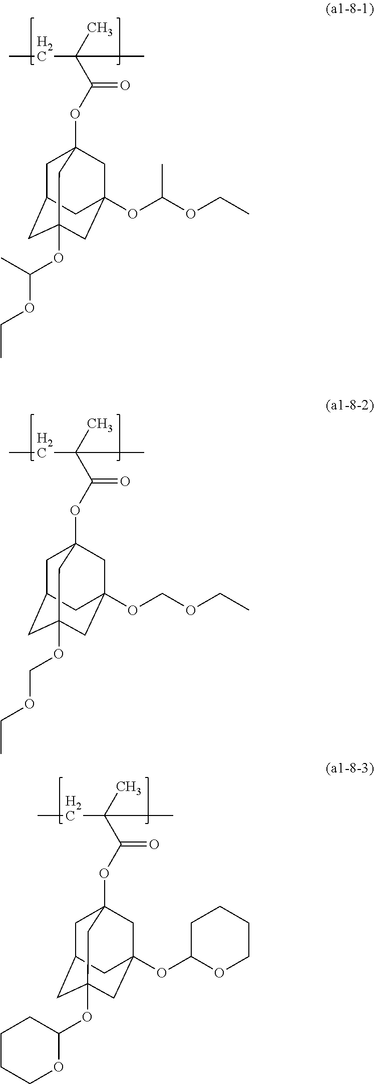

When the resin (A) and/or the like includes the structural units represented by formulas (a1-8-1) to (a1-8-3), the content ratio thereof, with respect to all structural units of the resin (A) and/or the like, is, for example, 10 mol % or more, preferably 15 mol % or more, more preferably 20 mol % or more, further preferably 25 mol % or more, still more preferably 30 mol % or more, still further preferably 40 mol % or more, and further more preferably 50 mol % or more. In addition, the content ratio is, for example, 95 mol % or less, preferably 90 mol % or less, more preferably 85 mol % or less, further preferably 80 mol % or less, still more preferably 75 mol % or less, still further preferably 70 mol % or less, and further more preferably 60 mol % or less. Specifically, the content ratio is preferably 10 to 60 mol %, more preferably 15 to 55 mol %, further preferably 20 to 50 mol %, further more preferably 20 to 45 mol %, and particularly preferably 20 to 40 mol %.

<Structural Unit (s)>

A structural unit (s) is derived from a monomer that does not have an acid labile group (hereinafter referred to as “monomer (s)” in some cases). The monomer from which the structural unit (s) is derived can be a monomer that does not have an acid labile group that is known in the resist field.

The structural unit (s) preferably has a hydroxy group, a carboxy group or a lactone ring. Using a resin that includes a structural unit that has a hydroxy group or a carboxy group and no acid labile group (hereinafter referred to as “structural unit (a2)” in some cases) and/or a structural unit that has a lactone ring and no acid labile group (hereinafter referred to as “structural unit (a3)” in some cases) in the resist composition of the present invention allows resolution of the resist pattern and adhesion to the substrate to be improved. In addition to the structural units described above, examples of the structural unit (s) include a structural unit that has a halogen atom (hereinafter referred to as “structural unit (a4)” in some cases), a structural unit that has a non-leaving hydrocarbon group (hereinafter referred to as “structural unit (a5)” in some cases), a structural unit that has a sultone structure (hereinafter referred to as “structural unit (a6)” in some cases), a structural unit that decomposes upon exposure to generate acid (hereinafter referred to as “structural unit (a7)” in some cases), and other structural units known in the art.

<Structural Unit (a2)>

The structural unit (a2) is a structural unit represented by formula (a2), and has an alcoholic hydroxy group, a phenolic hydroxy group or a carboxy group:

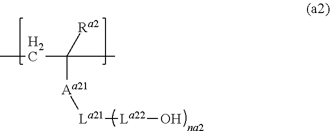

wherein
R^{a2} represents a hydrogen atom, a halogen atom or an alkyl group having 1 to 6 carbon atoms and optionally having a halogen atom;
A^{a21} represents a single bond or an alkanediyl group having 1 to 12 carbon atoms, and —CH₂— contained in the alkanediyl group is optionally replaced with —O—, —CO— or —NR^{a28}_;
R^{a28} represents a hydrogen atom or an alkyl group having 1 to 6 carbon atoms;
L^{a21} represents a single bond, or a hydrocarbon group having 1 to 28 carbon atoms, the hydrocarbon group optionally has a substituent, and —CH₂— contained in the hydrocarbon group is optionally replaced with —O—, —CO—, —S—, —SO—, —NR^{a28}— or —SO₂—;
L^{a22} represents a single bond or a chain hydrocarbon group having 1 to 12 carbon atoms and optionally having a fluorine atom, and —CH₂— contained in the chain hydrocarbon group is optionally replaced with —O— or —CO—;
na2 represents an integer of 1 to 5; and when na2 is 2 or larger, a plurality of L^{a22} may be the same or different from each other.

Examples of the halogen atom and the alkyl group optionally having a halogen atom in R^{a2} of formula (a2) include the same as those exemplified for R^{a01}, R^{a4} and R^{a5} in formula (a1-0) and the like.

R^{a2} is preferably a hydrogen atom or an alkyl group having 1 to 4 carbon atoms, is more preferably a hydrogen atom, a methyl group or an ethyl group, and is further preferably a hydrogen atom or a methyl group.

Examples of the alkanediyl group of A^{a21} include the same alkanediyl group as A^{a11} in formula (a1-4). The number of carbon atoms of the alkanediyl group is preferably 1 to 10, is more preferably 1 to 8, is further preferably 1 to 6, is still more preferably 1 to 4, is still further preferably 1 to 3, and is further more preferably 1 or 2.

Examples of the alkyl group of R^{a28} include the same alkyl group as R^{a18} in formula (a1-4).

A^{a21} is preferably a single bond, *—CO—O— or *—CO—O-A^{a22}-CO—O—, is more preferably a single bond, *—CO—O— or *—CO—O—CH₂—CO—O—, and is further preferably a single bond or *—CO—O—. Examples of the alkanediyl group of A^{a22} include the same alkanediyl group as A^{a21}, within the range permitted by the upper limit of the number of carbon atoms.

The hydrocarbon group in L^{a21} is a hydrocarbon group having a valence of (na2+1), and examples thereof include a straight-chain or branched chain hydrocarbon group, a monocyclic or polycyclic (including a spiro ring, a condensed ring or a bridged ring) alicyclic hydrocarbon group, and a cyclic hydrocarbon group such as an aromatic hydrocarbon group, and it may be a combined group of two or more of these groups (for example, a hydrocarbon group formed from an alicyclic hydrocarbon group or an aromatic hydrocarbon group and a chain hydrocarbon group).

Examples of the chain hydrocarbon group of L^{a21} include a divalent to hexavalent chain hydrocarbon group such as an alkanediyl group, an alkanetriyl group, an alkanetetrayl group, an alkanepentayl group and an alkanehexayl groups.

Examples of the alkanediyl group include the same alkanediyl group as A^{a21}.

Examples of the alkanetriyl group include a methanetriyl group, an ethanetriyl group, a propanetriyl group, a butanetriyl group, a pentanetriyl group, a hexanetriyl group, a heptanetriyl group, an octanetriyl group, a nonanetriyl group, a decanetriyl group, an undecanetriyl group, a dodecanetriyl group, a tridecanetriyl group, a tetradecanetriyl group, a pentadecanetriyl group, a hexadecanetriyl group and a heptadecanetriyl group.

Examples of the alkanetetrayl group include a methanetetrayl group, an ethanetetrayl group, a propanetetrayl group, a butanetetrayl group, a pentanetetrayl group, a hexanetetrayl group, a heptanetetrayl group, an octanetetrayl group, a nonanetetrayl group, a decanetetrayl group, an undecanetetrayl group, a dodecanetetrayl group, a tridecanetetrayl group, a tetradecanetetrayl group, a pentadecanetetrayl group, a hexadecanetetrayl group and a heptadecanetetrayl group.

Examples of the alkanepentayl group include an ethanepentayl group, a propanepentayl group, a butanepentayl group, a pentanepentayl group and a hexanepentayl group.

Examples of the alkanehexayl group include an ethanehexayl group, a propanehexayl group, a butanehexayl group, a pentanehexayl group and a hexanehexayl group.

In addition, examples thereof also include a group in which one or more hydrogen atoms of the above groups are replaced with bonding sites.

The number of carbon atoms of the chain hydrocarbon group of L^{a21} is preferably 1 to 18, is more preferably 1 to 12, is further preferably 1 to 10, is still more preferably 1 to 9, is still further preferably 1 to 8, is further more preferably 1 to 6, is still even more preferably 1 to 5, and is particularly preferably 1 to 4.

Examples of the monocyclic and polycyclic alicyclic hydrocarbon group in L^{a21} include the following alicyclic hydrocarbon groups. The bonding site can be set to any position.

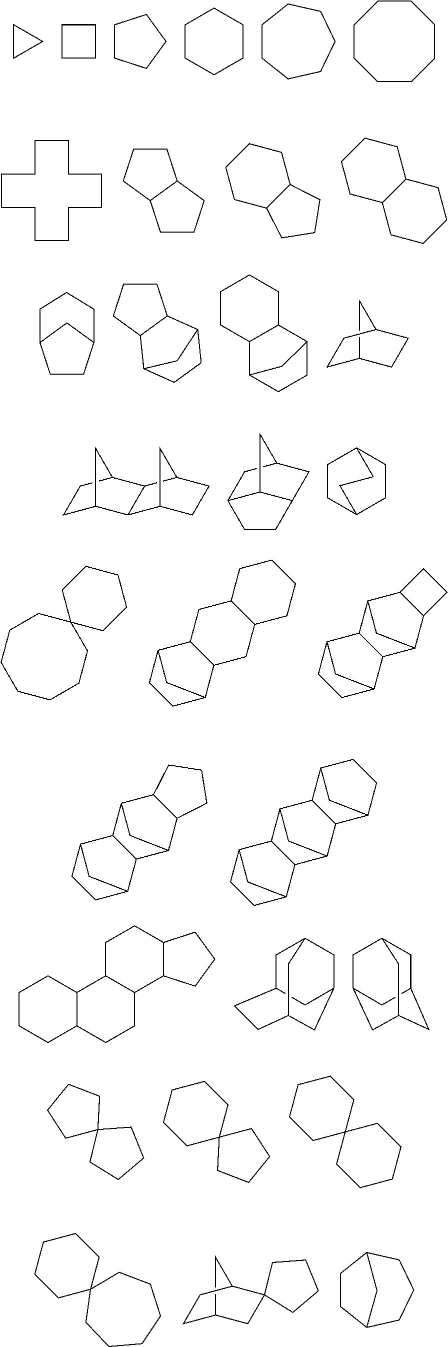

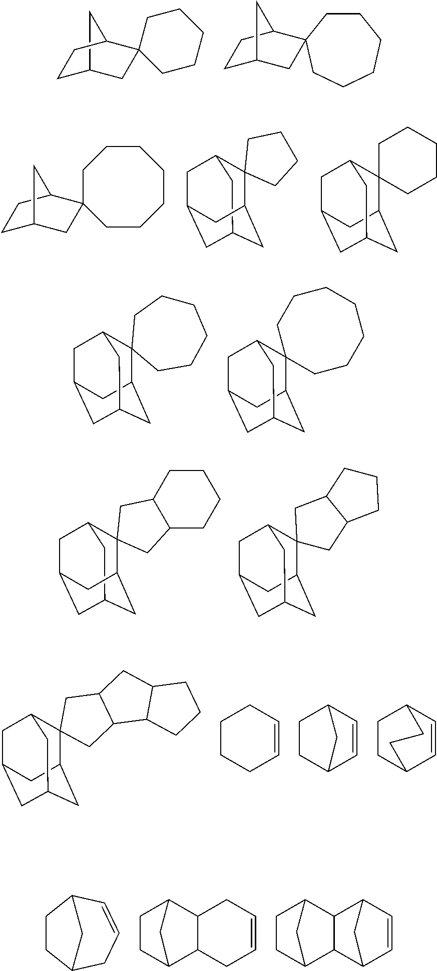

Examples thereof include a divalent to hexavalent alicyclic hydrocarbon group such as a cycloalkanediyl group, a cycloalkanetriyl group, a cycloalkanetetrayl group, a cycloalkanepentayl group and a cycloalkanehexayl group.

Specific examples thereof include a monocyclic alicyclic hydrocarbon group such as a cyclobutane-1,3-diyl group, a cyclopentane-1,3-diyl group, a cyclohexane-1,4-diyl group, a cyclooctane-1,5-diyl group, a cyclopropanetriyl group, a cyclobutanetriyl group, a cyclopentanetriyl group, a cyclohexanetriyl group, a cycloheptanetriyl group, a cyclooctanetriyl group, a cyclodecanetriyl group, a cyclopropanetetrayl group, a cyclobutanetetrayl group, a cyclopentanetetrayl group, a cyclohexanetetrayl group, a cycloheptanetetrayl group, a cyclooctanetetrayl group and a cyclodecanetetrayl group, and a polycyclic alicyclic hydrocarbon group such as a norbornane-1,4-diyl group, a norbornane-2,5-diyl group, an adamantane-1,5-diyl group, an adamantane-2,6-diyl group, a decahydronaphthalenediyl group, a bicyclo[3.3.0]octanediyl group, a norbornanetriyl group, an adamantanetriyl group, a decahydronaphthalenetriyl group, a bicyclo[3.3.0]octanetriyl group, a norbornanetetrayl group, an adamantanetetrayl group, a decahydronaphthalenetetrayl group and a bicyclo[3.3.0]octanetetrayl group.

In addition, examples thereof also include a group in which one or more hydrogen atoms of the above groups are replaced with bonding sites.

The number of carbon atoms of the alicyclic hydrocarbon group is preferably 3 to 20, is more preferably 3 to 18, is further preferably 3 to 16, is still more preferably 3 to 12, is still further preferably 6 to 12, and is particularly preferably 6 to 10.

Examples of the aromatic hydrocarbon group in L^{a21} include a divalent to hexavalent aromatic hydrocarbon group such as an arylene group, an arenetriyl group, an arenetetrayl group, an arenepentayl group and an arenehexayl group.

Specific examples thereof include a phenylene group, a naphthylene group, an anthrylene group, a biphenylene group, a phenanthrylene group, a benzenetriyl group, a naphthalenetriyl group, an anthracenetriyl group, a biphenylenetriyl group, a phenanthrenetriyl group, a benzenetetrayl group, a naphthalenetetrayl group, an anthracenetetrayl group, a biphenylenetetrayl group and a phenanthrenetetrayl group.

In addition, examples thereof also include a group in which one or more hydrogen atoms of the above groups are replaced with bonding sites.

The number of carbon atoms of the aromatic hydrocarbon group is preferably 6 to 20, is more preferably 6 to 18, is further preferably 6 to 14, is still more preferably 6 to 12, and is still further preferably 6 to 10.

Examples of the combined group of two or more include a combined group of an alicyclic hydrocarbon group and a chain hydrocarbon group, a combined group of an aromatic hydrocarbon group and a chain hydrocarbon group, a combined group of an alicyclic hydrocarbon group and an aromatic hydrocarbon group, and a combined group of an alicyclic hydrocarbon group, a chain hydrocarbon group and an aromatic hydrocarbon group. In the combination, two or more types of alicyclic hydrocarbon groups, aromatic hydrocarbon groups, and chain hydrocarbon groups may each be combined. In addition, any of the groups may be bonded to A^{a21} and L^{a22}.

Examples of the group formed by replacing —CH₂— contained in the hydrocarbon group with —O—, —S—, —SO₂—, —SO—, —NR^{a28} or —CO— include a hydroxy group, a carboxy group, a thiol group, an amino group, an alkoxy group, an alkylthio group, an alkoxycarbonyl group, an alkylsulfonyl group, an alkylcarbonyl group, an alkylcarbonyloxy group, an alkoxycarbonyloxy group, an alkoxyalkoxy group, an alkylamino group, an alkyl peptide group, an oxy group, a carbonyl group, a thio group, a sulfonyl group, a peptide group, an alkanediyl oxy group, an alkanediyloxycarbonyl group, an alkanediylcarbonyl group, an alkanediylcarbonyloxy group, an alkanediyloxycarbonyloxy group, an alkanediylsulfonyl group, an alkanediylthio group, an alkanediyl amino group, an alkanediyl peptide group, a cycloalkoxy group, a cycloalkylalkoxy group, an aromatic hydrocarbon group -carbonyloxy group, an aromatic hydrocarbon group-carbonyl group, an aromatic hydrocarbon group-oxy group and a combined group of two or more of these groups. In addition, examples thereof also include a group in which one or more hydrogen atoms of these groups are replaced with bonding sites. Examples of these groups formed by the replacement include the same groups as those exemplified in the present specification, within the range permitted by the upper limit of the number of carbon atoms.

In addition, examples of the group formed by replacing —CH₂-contained in the alicyclic hydrocarbon group, the aromatic hydrocarbon group or the combined group of these groups with —O—, —S—, —CO—, —SO—, —NR^{a28}— or —SO₂— include the same groups as those exemplified in the present specification, within the range permitted by the upper limit of the number of carbon atoms.

The hydrocarbon group in L^{a21} optionally has one or more substituents. Examples of the substituent include a halogen atom, a haloalkyl group having 1 to 12 carbon atoms, an alkyl group having 1 to 16 carbon atoms, where —CH₂— contained in the alkyl group is optionally replaced with —O— or —CO—, an acryloyloxy group and a methacryloyloxy group.

Examples of the halogen atom include a fluorine atom, a chlorine atom, a bromine atom and an iodine atom.

The hydrocarbon group in L^{a21} can substantially have a substituent such as a haloalkyl group by having a halogen atom as a substituent. Examples of the haloalkyl group include a fluorinated alkyl group, a chlorinated alkyl group, a brominated alkyl group and an iodinated alkyl group, and examples thereof include a chloromethyl group, a bromomethyl group, an iodomethyl group, a fluoromethyl group, a difluoromethyl group, a trifluoromethyl group, a perfluoroethyl group, a perfluoropropyl group and a perfluorobutyl group. The number of carbon atoms of the haloalkyl group is preferably 1 to 8, is more preferably 1 to 6, is further preferably 1 to 4, and is further more preferably 1 to 3.

When the hydrocarbon group of L^{a21} includes a branched structure, L^{a21} can substantially have a substituent such as an alkyl group. Examples of the alkyl group include a methyl group, an ethyl group, a n-propyl group, an isopropyl group, a n-butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group. The number of carbon atoms of the alkyl group is preferably 1 to 12, is more preferably 1 to 10, is further preferably 1 to 8, is still more preferably 1 to 6, is still further preferably 1 to 4, and is further more preferably 1 to 3.

When —CH₂— contained in the hydrocarbon group of L^{a21} is replaced with —O—, —S—, —SO—, —NR^{a28}—, —SO₂— or —CO—, then L^{a21} can substantially have a substituent such as a hydroxy group, a carboxy group, an alkoxy group, an alkoxycarbonyl group, an alkylcarbonyl group, an alkylcarbonyloxy group, a thiol group, an alkylthio group, an alkylsulfonyl group, an alkoxyalkyl group, an alkoxyalkoxy group, an alkoxycarbonyloxy group, an amino group, an alkylamino group, an alkyl peptide group, an acryloyloxy group and a methacryloyloxy group. Examples of such groups include the same groups as those exemplified in the present specification, within the range permitted by the upper limit of the number of carbon atoms.

The substituent that the hydrocarbon group in L^{a21} optionally has is preferably a halogen atom, a haloalkyl group having 1 to 8 carbon atoms or an alkyl group having 1 to 12 carbon atoms, where —CH₂— contained in the alkyl group is optionally replaced with —O— or —CO—, is more preferably a halogen atom, a haloalkyl group having 1 to 6 carbon atoms or an alkyl group having 1 to 10 carbon atoms, where —CH₂— contained in the alkyl group is optionally replaced with —O— or —CO—, is further preferably a halogen atom, a haloalkyl group having 1 to 4 carbon atoms, an alkyl group having 1 to 4 carbon atoms, a hydroxy group, an alkoxy group having 1 to 4 carbon atoms or an alkoxyalkoxy group having 2 to 8 carbon atoms, is still more preferably a fluorine atom, an iodine atom, a trifluoromethyl group, a methyl group, a hydroxy group, a methoxy group, an ethoxy group, an ethoxyethoxy group or an ethoxymethoxy group, and is still further preferably a fluorine atom, an iodine atom, a trifluoromethyl group, a methyl group, a hydroxy group, a methoxy group or an ethoxyethoxy group.

In addition, when the alkanediyl group of L^{a21} is replaced with —O—, —CO— or the like, for example, *-L^{a23}-X^{a21}—, where L^{a23} represents an alkanediyl group having 1 to 8 carbon atoms, X^{a21} represents —O—, —O—CO—, —CO—O— or —O—CO—O—, and * represents a bonding site to A^{a21}, is also preferable.

L^{a21} is preferably a single bond, a chain hydrocarbon group having 1 to 12 carbon atoms and optionally having a substituent, where —CH₂— contained in the chain hydrocarbon group is optionally replaced with —O— or —CO—, a cyclic hydrocarbon group having 3 to 20 carbon atoms and optionally having a substituent, where —CH₂— contained in the cyclic hydrocarbon group is optionally replaced with —O—, —S—, —CO—, —SO—, —NR^{a28} or —SO₂—, or a group formed by combining a chain hydrocarbon group having 1 to 8 carbon atoms and optionally having a substituent and a cyclic hydrocarbon group having 3 to 20 carbon atoms and optionally having a substituent, where —CH₂— contained in the chain hydrocarbon group is optionally replaced with —O— or —CO—, and —CH₂— contained in the cyclic hydrocarbon group is optionally replaced with —O—, —S—, —SO₂—, —SO—, —NR^{a28}— or —CO—, and is more preferably a chain hydrocarbon group having 1 to 10 carbon atoms and optionally having a substituent, where —CH₂— contained in the chain hydrocarbon group is optionally replaced with —O— or —CO—, a cyclic hydrocarbon group having 3 to 18 carbon atoms and optionally having a substituent, where —CH₂— contained in the cyclic hydrocarbon group is optionally replaced with —O—, —S—, —CO—, —SO—, —NR^{a28} or —SO₂—, or a group formed by combining a chain hydrocarbon group having 1 to 6 carbon atoms and optionally having a substituent and a cyclic hydrocarbon group having 3 to 18 carbon atoms and optionally having a substituent, where —CH₂— contained in the chain hydrocarbon group is optionally replaced with —O— or —CO—, and —CH₂— contained in the cyclic hydrocarbon group is optionally replaced with —O—, —S—, —SO₂—, —SO—, —NR^{a21}_or —CO—.

Examples of the chain hydrocarbon group having 1 to 12 carbon atoms of L^{a22} include the same alkanediyl groups as those exemplified for A^{a11} in formula (a1-4). The number of carbon atoms of the chain hydrocarbon group is preferably 1 to 10, is more preferably 1 to 8, is further preferably 1 to 6, and is still more preferably 1 to 4.

Examples of the group formed by replacing —CH₂— contained in the chain hydrocarbon group with —O—, —S—, —CO—, —SO— or —SO₂— include the same as those exemplified in the present specification, within the range permitted by the upper limit of the number of carbon atoms.

L^{a22} optionally has one fluorine atom or two or more fluorine atoms.

na2 is preferably an integer of 1 to 4, and is more preferably an integer of 1 to 3.

Examples of the structural unit (a2) when L^{a21} is a single bond or a chain hydrocarbon group include the following structural units. Similarly to the following structural units, structural units in which the methyl group corresponding to R^{a2} in the following structural units is replaced with a hydrogen atom or the like are also suitable structural units for the structural unit (a2).

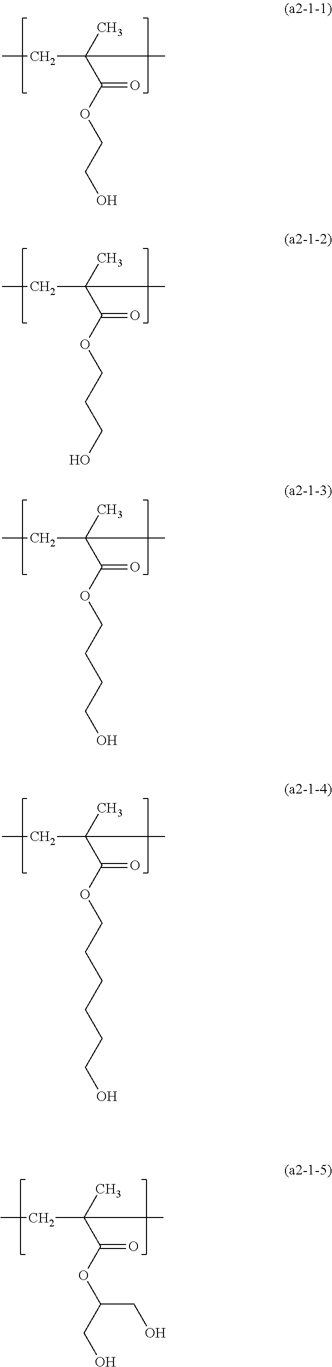

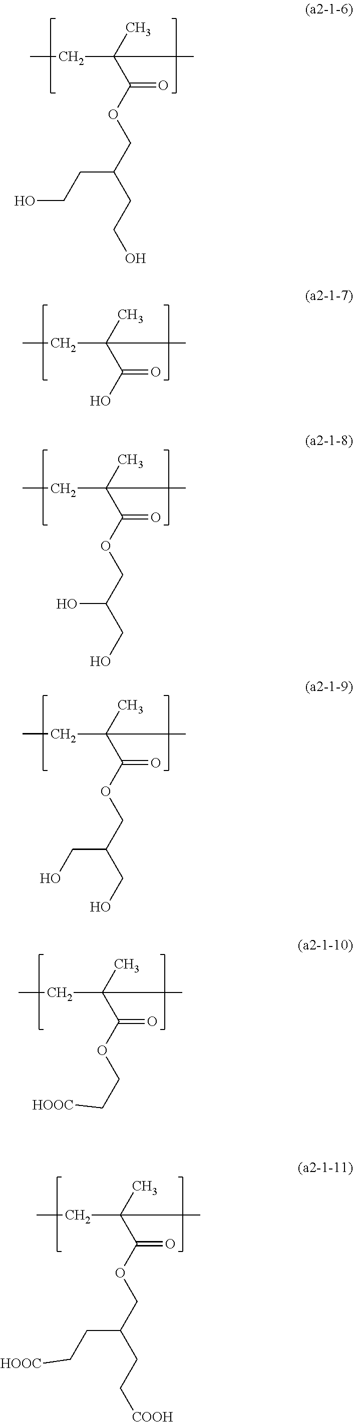

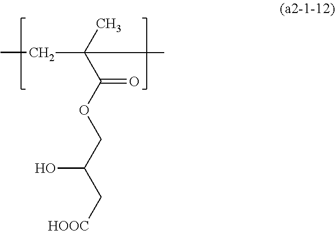

When producing a resist pattern from the resist composition of the present invention and a high-energy ray such as a KrF excimer laser (248 nm), an electron beam, or EUV (extreme ultraviolet) is used as the exposure light source, the structural unit (a2) is preferably a structural unit (a2) having a phenolic hydroxy group, and it is more preferable to use the structural unit (a2-A) described below. In addition, when an ArF excimer laser (193 nm) or the like is used, the structural unit (a2) is preferably a structural unit (a2) having an alcoholic hydroxy group, and it is more preferable to use the structural unit (a2-C) described below. The structural unit (a2) may include one type singly, or two or more types.

In the structural unit (a2), when L^{a21} is a cyclic hydrocarbon group, the structural unit having a phenolic hydroxy group or a carboxy group is a structural unit represented by formula (a2-A) (hereinafter referred to as “structural unit (a2-A)” in some cases):

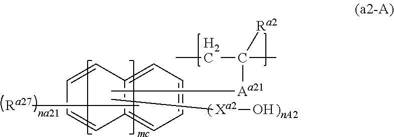

wherein all symbols in formula (a2-A) have the same meanings as described above.

Examples of R^{a2} and A^{a21} each include the same groups as those exemplified in formula (a2).

Examples of R^{a27} include the same groups as those exemplified for R^{a17} in formula (a1-4). The number of carbon atoms of the alkoxy group is preferably 1 to 4, and is more preferably 1 to 3. The alkoxy group is preferably an alkoxy group having 1 to 4 carbon atoms, is more preferably an alkoxy group having 1 to 3 carbon atoms, is further preferably a methoxy group or an ethoxy group, and is further more preferably a methoxy group. The number of carbon atoms of the alkoxyalkyl group is preferably 2 to 8, and is more preferably 2 to 4. The alkoxyalkyl group is preferably an alkoxyalkyl group having 2 to 8 carbon atoms, is more preferably an alkoxyalkyl group having 2 to 4 carbon atoms, is further preferably a methoxymethyl group or an ethoxyethyl group, and is further more preferably a methoxymethyl group. The number of carbon atoms of the alkoxyalkoxy group is preferably 2 to 8, and is more preferably 2 to 4. The alkoxyalkoxy group is preferably an alkoxyalkoxy group having 2 to 8 carbon atoms, is more preferably an alkoxyalkoxy group having 2 to 4 carbon atoms, and is further preferably a methoxyethoxy group or an ethoxyethoxy group. The alkylcarbonyl group is preferably an alkylcarbonyl group having 2 to 4 carbon atoms, is more preferably an alkylcarbonyl group having 2 to 3 carbon atoms, and is further preferably an acetyl group. The number of carbon atoms of the alkylcarbonyloxy group is preferably 2 to 4, and is more preferably 2 to 3. The alkylcarbonyloxy group is preferably an alkylcarbonyloxy group having 2 to 4 carbon atoms, is more preferably an alkylcarbonyloxy group having 2 to 3 carbon atoms, and is further preferably an acetyloxy group.

R^{a27} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, is more preferably a fluorine atom, an iodine atom, a hydroxy group, a methyl group, a methoxy group, an ethoxy group, an ethoxyethoxy group or an ethoxymethoxy group, and is further preferably a fluorine atom, an iodine atom, a hydroxy group, a methyl group, a methoxy group or an ethoxyethoxy group.
nA2 is preferably 1, 2, 3 or 4, is more preferably 1, 2 or 3, and is further preferably 1 or 2.
na21 is preferably 0, 1, 2 or 3, is more preferably 0, 1 or 2, and is further preferably 0 or 1.
mc is preferably 0 or 1.

In the case of a benzene ring, —X^{a2}—OH may be bonded to any of the o-position, m-position or p-position with respect to the bonding position of A^{a21}. In particular, at least one is preferably bonded to the m-position or p-position, and more preferably bonded to the m-position. In the case of a naphthalene ring, when A^{a21} is bonded to the 1st position, the above group may be bonded to any of the 2nd to 8th positions, and when A^{a21} is bonded to the 2nd position, the above group may be bonded to any of the 1st and 3rd to 8th positions. In particular, when A^{a21} is bonded to the 1st position, at least one is preferably bonded to the 3rd to 6th positions, and more preferably bonded to the 3rd or 4th position. When A^{a21} is bonded to the 2nd position, the above group is preferably bonded to the 4th to 7th positions, and more preferably bonded to the 5th or 6th position.

Examples of the structural unit (a2-A) include structural units derived from monomers disclosed in Japanese Patent Laid-Open No. 2010-204634 and Japanese Patent Laid-Open No. 2012-12577.

Examples of the structural unit (a2-A) include structural units represented by formulas (a2-2-1) to (a2-2-32) and (a2-3-1) to (a2-3-24), and structural units represented by formulas (a2-2-1) to (a2-2-32) and (a2-3-1) to (a2-3-24) in which the methyl group corresponding to R^{a2} in the structural unit (a2-A) is replaced with a hydrogen atom, a halogen atom, a haloalkyl group or another alkyl group.

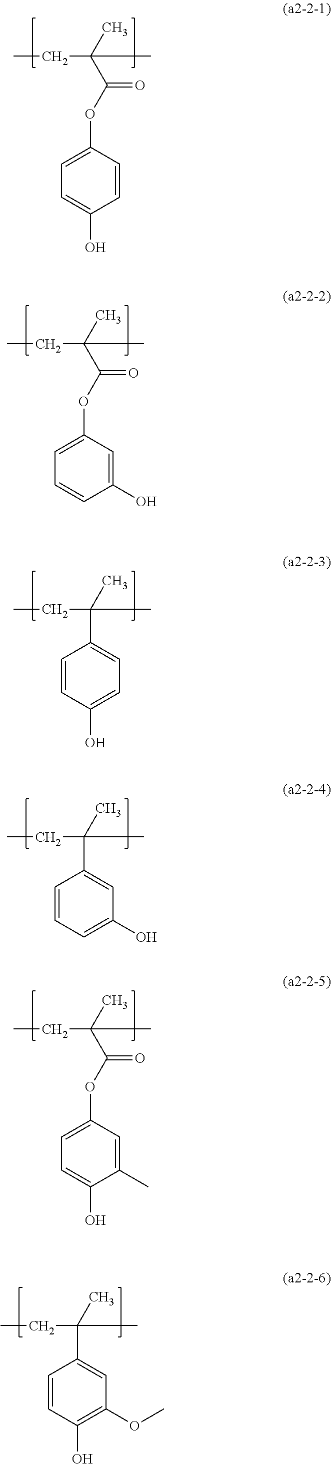

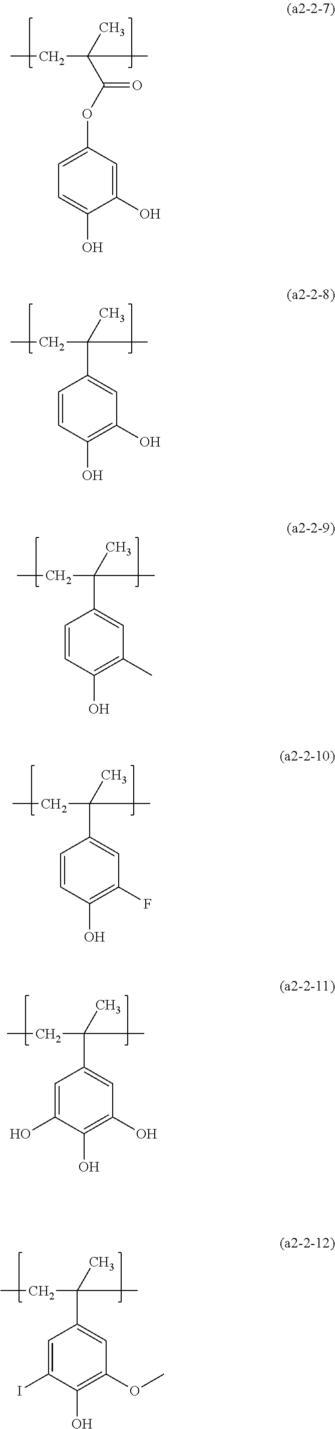

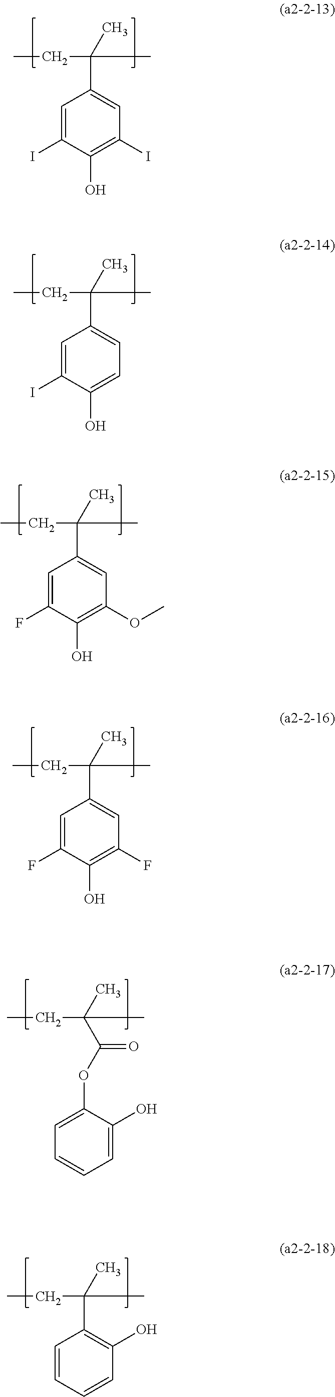

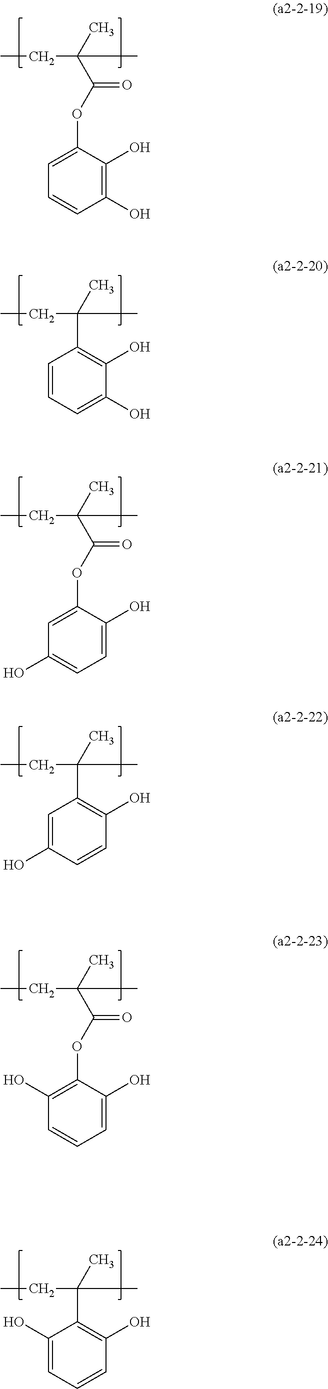

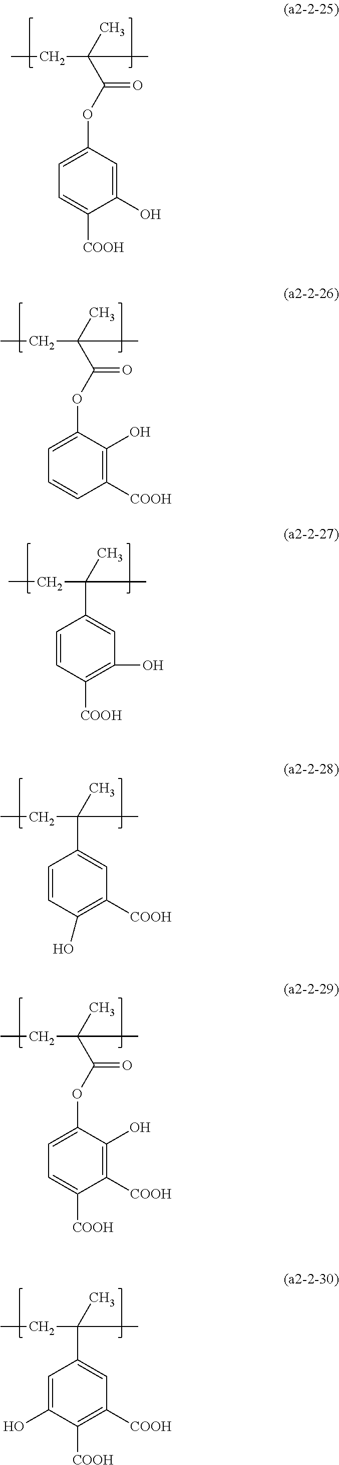

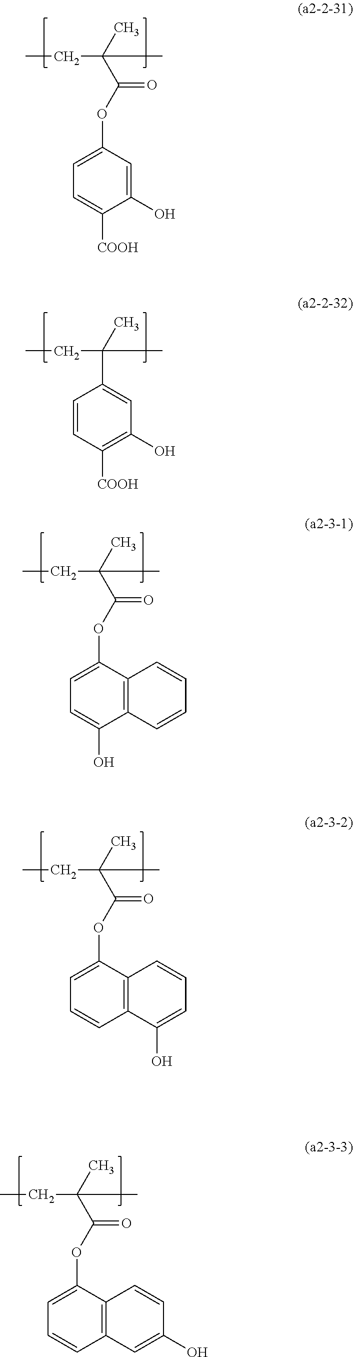

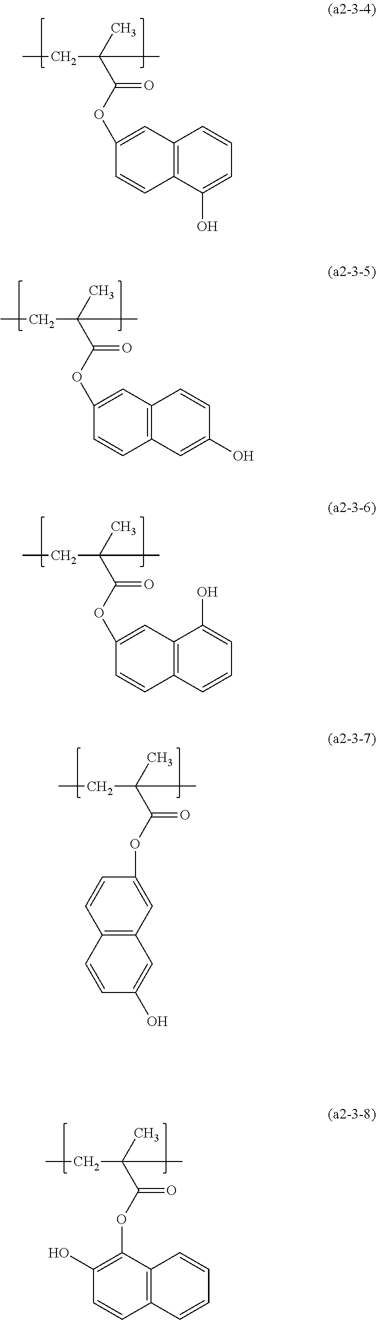

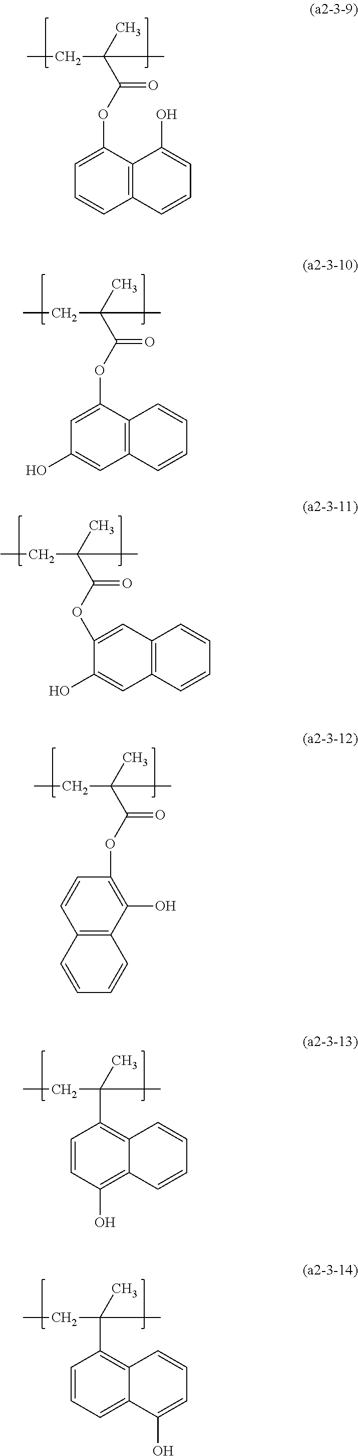

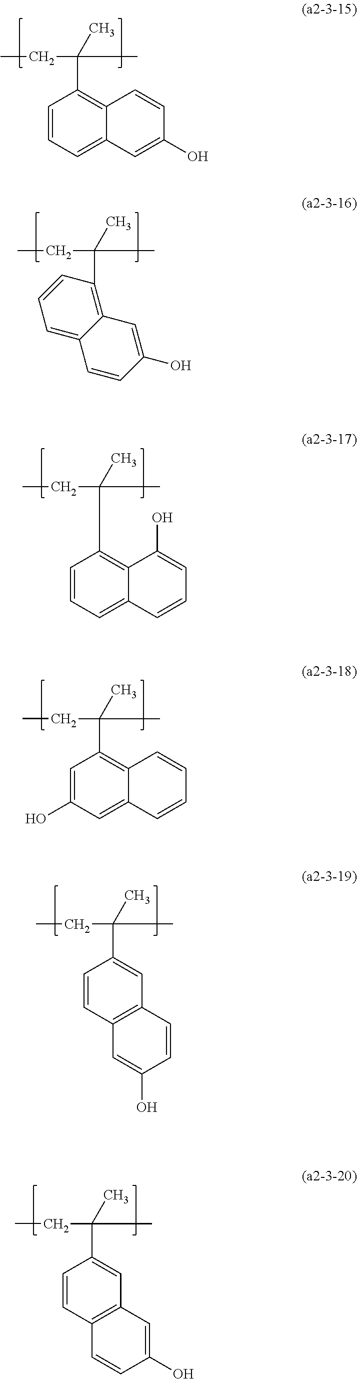

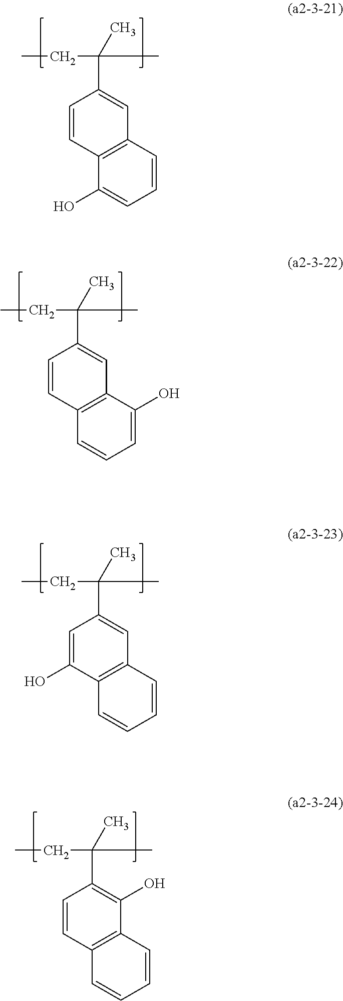

When the structural unit (a2-A) is included in the resin (A) and/or the like, the content ratio of the structural unit (a2-A), with respect to all structural units, is preferably 5 mol % or more, more preferably 10 mol % or more, further preferably 15 mol % or more, and still more preferably 20 mol % or more. In addition, the content ratio is preferably 80 mol % or less, more preferably 70 mol % or less, further preferably 65 mol % or less, still more preferably 60 mol % or less, still further preferably 50 mol % or less, further more preferably 45 mol % or less, and still even more preferably 40 mol % or less. Specifically, the content ratio is preferably 5 to 80 mol %, more preferably 10 to 70 mol %, further preferably 15 to 65 mol %, and still more preferably 20 to 65 mol %.

The structural unit (a2-A) is derived from a compound represented by formula (a2-A′) (hereinafter referred to as “compound (a2-A′)” in some cases):

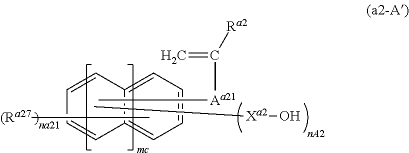

wherein all symbols in formula (a2-A′) have the same meanings as described above.

The compound (a2-A′) may be a commercially available product or one produced by a known method.

The structural unit (a2-A) can be included in the resin (A) and/or the like by polymerizing the compound (a2-A′). In addition, for example, it can be included in the resin (A) and/or the like by performing polymerization using the structural unit (a1-4), followed by treatment with an acid such as p-toluenesulfonic acid. In addition, the structural unit (a2-A) can be included in the resin (A) and/or the like by performing polymerization using acetoxystyrene or the like, followed by treatment with an alkali such as tetramethylammonium hydroxide.

In the structural unit (a2), when L^{a21} is a cyclic hydrocarbon group, examples of the structural unit having an alcoholic hydroxy group or a carboxy group include a structural unit represented by formula (a2-B) (hereinafter referred to as “structural unit (a2-B)” in some cases), a structural unit represented by formula (a2-C) (hereinafter referred to as “structural unit (a2-C)” in some cases), and a structural unit represented by formula (a2-D) (hereinafter referred to as “structural unit (a2-D)” in some cases):

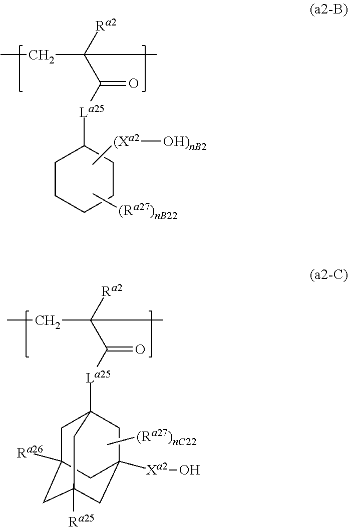

wherein
R^{a2} has the same meaning as in formula (a2);
R^{a27} has the same meaning as in formula (a2-A);
L^{a25} represents —O— or *—O—(CH₂)ₖ₂—CO—O—, where k2 represents an integer of 1 to 7, and * represents a bonding site to —CO—;
X^{a2} represents a single bond or —CO—;
R^{a25} and R^{a26} each independently represent a hydrogen atom, a methyl group or a hydroxy group;
nB2 represents an integer of 1 to 5, and when nB2 is 2 or larger, a plurality of X^{a2} may be the same or different from each other;
nB22 represents an integer of 0 to 8, and when nB22 is 2 or more, a plurality of R^{a27} may be the same or different from each other; and
nC22 represents an integer of 0 to 10, and when nC22 is 2 or more, a plurality of R^{a27} may be the same or different from each other.
L^{a25} is preferably —O— or —O—(CH₂)_{f1}—CO—O—, where f1 represents an integer of 1 to 4, and is more preferably —O—.
R^{a2} is preferably a methyl group.
X^{a2} is preferably a single bond.
R^{a25} is preferably a hydrogen atom.
R^{a26} is preferably a hydrogen atom or a hydroxy group.
R^{a27} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, is more preferably a fluorine atom, an iodine atom, a hydroxy group, a methyl group, a methoxy group, an ethoxy group, an ethoxyethoxy group or an ethoxymethoxy group, and is further preferably a fluorine atom, an iodine atom, a hydroxy group, a methyl group, a methoxy group or an ethoxyethoxy group.

nB2 is preferably 1, 2, 3 or 4, is more preferably 1, 2 or 3, and is further preferably 1 or 2.

nB22 is preferably an integer of 0 to 3, is more preferably 0, 1 or 2, and is further preferably 0 or 1.

nC22 is preferably an integer of 0 to 6, is more preferably an integer of 0 to 3, is further preferably 0, 1 or 2, and is still more preferably 0 or 1.

Examples of the structural unit (a2-B) and the structural unit (a2-C) include a structural unit derived from a monomer disclosed in Japanese Patent Laid-Open No. 2010-204646, the following structural units, and structural units in which the methyl group or hydrogen atom corresponding to R^{a2} in the following structural units is replaced with a hydrogen atom, a halogen atom, a haloalkyl group or another alkyl group. Among these, structural units represented by any of formulas (a2-B-1) to (a2-B-5) and formulas (a2-C-1) to (a2-C-9) are preferable.

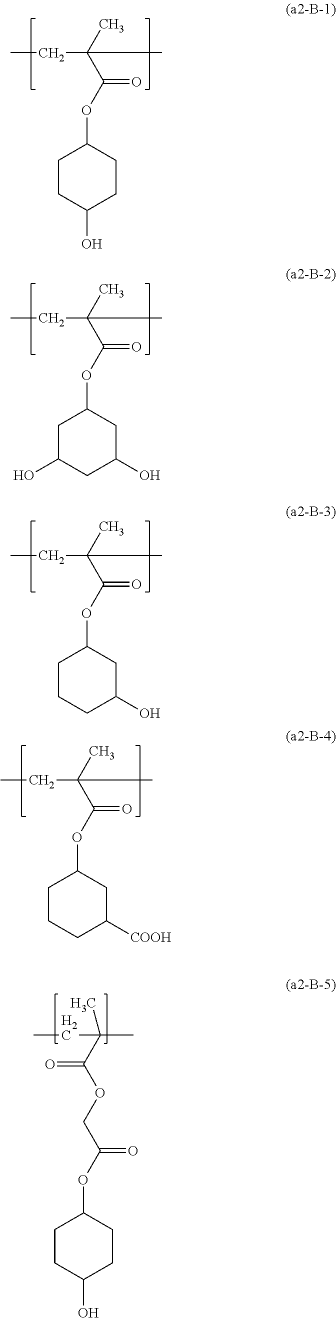

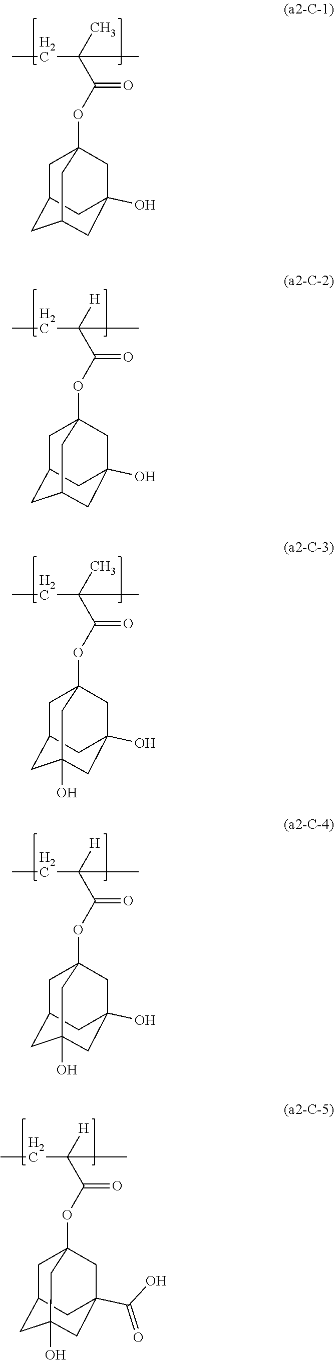

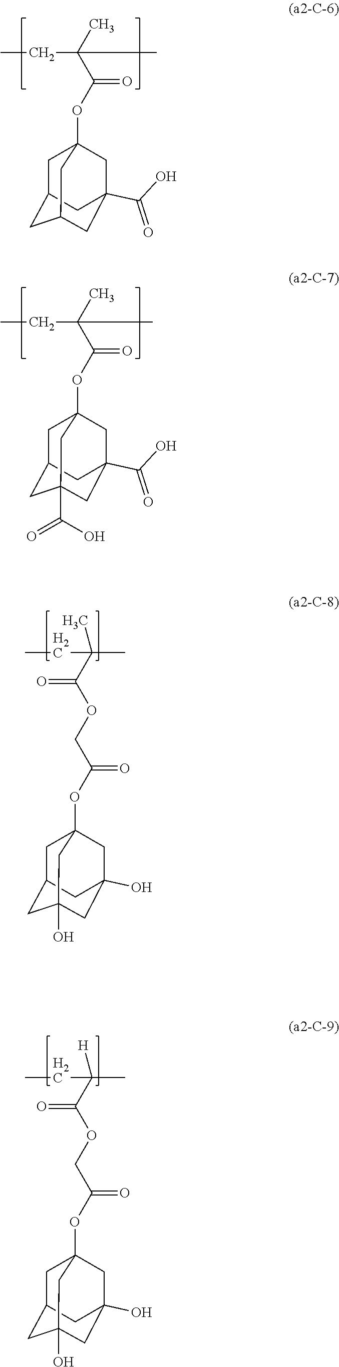

When the resin (A) and/or the like includes the structural unit (a2-B) or the structural unit (a2-C), the content ratio thereof, with respect to all structural units of the resin (A) and/or the like, is, for example, 1 mol % or more, and preferably 2 mol % or more. In addition, the content ratio is, for example, 45 mol % or less, preferably 40 mol % or less, more preferably 35 mol % or less, further preferably 20 mol % or less, and still more preferably 10 mol % or less. Specifically, the content ratio is, for example, 1 to 45 mol %, preferably 1 to 40 mol %, more preferably 1 to 35 mol %, further preferably 1 to 20 mol %, and further more preferably 1 to 10 mol %.

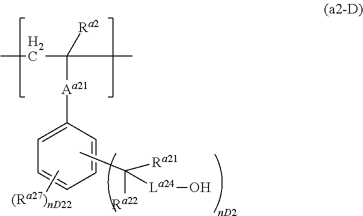

wherein
R^{a2} and A^{a21} each have the same meaning as in formula (a2);
R^{a27} has the same meaning as in formula (a2-A);
R^{a21} and R^{a22} each independently represent a fluorinated alkyl group having 1 to 4 carbon atoms;
L^{a24} represents a single bond or an alkanediyl group having 1 to 3 carbon atoms and the alkanediyl group may be substituted with a fluorine atom;
nD2 represents an integer of 1 to 5, and when nD2 is 2 or larger, a plurality of groups in parentheses may be the same or different from each other; and
nD22 represents an integer of 0 to 4, and when nD22 is 2 or larger, a plurality of R^{a27} may be the same or different from each other,
provided that 1≤nD2+nD22≤5.

Examples of the fluorinated alkyl group of R^{a21} and R^{a22} each independently include a trifluoromethyl group, a 2,2,2-trifluoroethyl group, a 3,3,3-trifluoropropyl group and a 4,4,4-trifluorobutyl group.

R^{a21} and R^{a22} are preferably trifluoromethyl groups.

Examples of the alkanediyl group of L^{a24} include a methylene group, an ethane-1,1-diyl group, a propane-1,1-diyl group and a propane-2,2-diyl group.

L^{a24} is preferably a single bond or a methylene group.

R^{a27} is preferably a halogen atom, a hydroxy group, a carboxy group, an alkyl group having 1 to 4 carbon atoms and optionally having a halogen atom, an alkoxy group having 1 to 4 carbon atoms or an alkoxyalkoxy group having 2 to 8 carbon atoms, is more preferably a fluorine atom, an iodine atom, a hydroxy group, a methyl group, a methoxy group, an ethoxy group, an ethoxyethoxy group or an ethoxymethoxy group, and is further preferably a fluorine atom, an iodine atom, a hydroxy group, a methyl group, a methoxy group or an ethoxyethoxy group.

nD2 is preferably 1, 2, 3 or 4, is more preferably 1, 2 or 3, is further preferably 1 or 2, and is further more preferably 1. In addition, preferably, nD2 is 1 and the group in parentheses is bonded to the para position.

nD22 is preferably an integer of 0 to 3, is more preferably an integer of 0 to 2, is further preferably 0 or 1, and is further more preferably 0.

It is more preferable that the structural unit (a2-D) is a structural unit represented by the following formula (a2-D1) (hereinafter referred to as “structural unit (a2-D1)” in some cases):

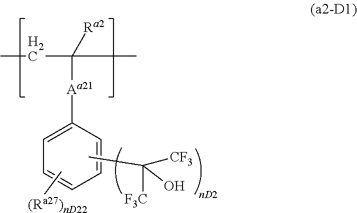

wherein
R^{a2}, A^{a21}, R^{a27}, nD2 and nD22 have the same meanings as in formula (a2-D).

In formula (a2-D1), R^{a2} is preferably a hydrogen atom or a methyl group.

A^{a21} is preferably a single bond.

R^{a27} is preferably a halogen atom, a hydroxy group, a carboxy group, an alkyl group having 1 to 4 carbon atoms, a haloalkyl group having 1 to 4 carbon atoms, an alkoxy group having 1 to 4 carbon atoms or an alkoxyalkoxy group having 2 to 8 carbon atoms, is more preferably a fluorine atom, an iodine atom, a hydroxy group, a methyl group, a methoxy group, an ethoxy group, an ethoxyethoxy group or an ethoxymethoxy group, and is further preferably a fluorine atom, an iodine atom, a hydroxy group, a methyl group, a methoxy group or an ethoxyethoxy group.

nD22 is preferably 0, 1, 2 or 3, is more preferably 0, 1 or 2, and is further preferably 0 or 1.

nD2 is preferably 1, 2, 3 or 4, is more preferably 1, 2 or 3, and is further preferably 1 or 2.

Examples of the structural unit (a2-D) include the structural units shown below. Structural units in which the hydrogen atom corresponding to R^{a2} in the following structural units is replaced with a methyl group or the like, and structural units in which the methyl group is replaced with a hydrogen atom or the like can also be mentioned as specific examples of the structural unit (a2-D). Among these, the structural units represented by formulas (a2-D-1) to (a2-D-8) are preferable, the structural units represented by formulas (a2-D-1) to (a2-D-4) are more preferable, and the structural unit represented by formula (a2-D 1) is further preferable.

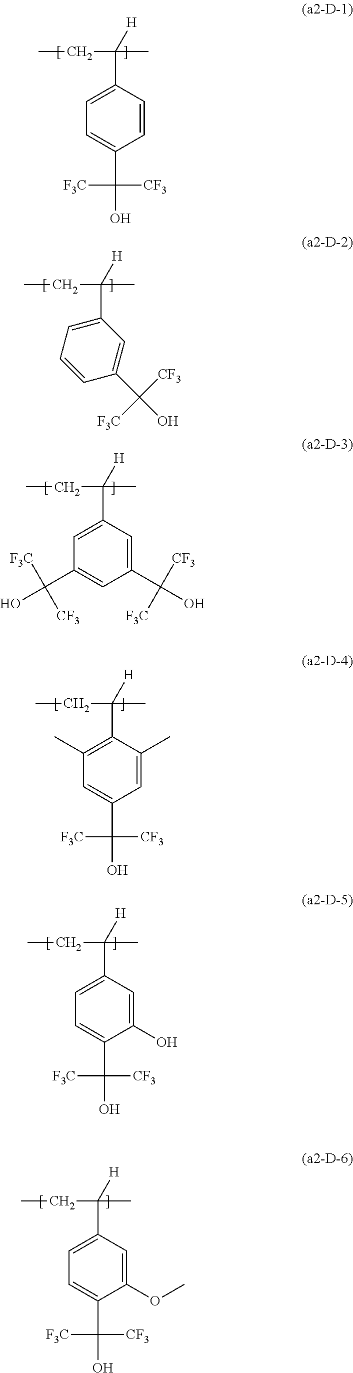

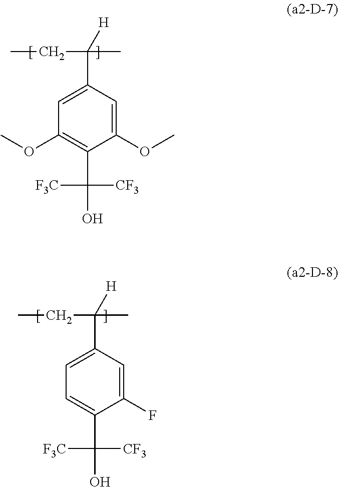

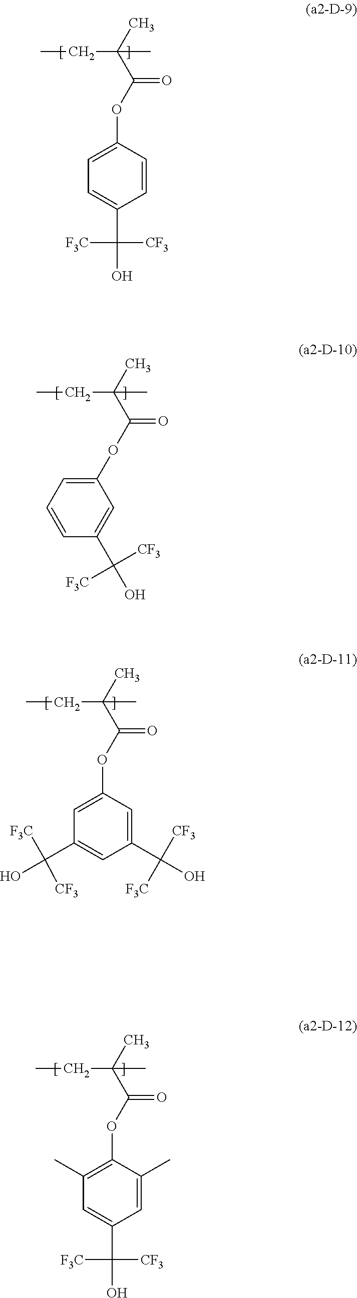

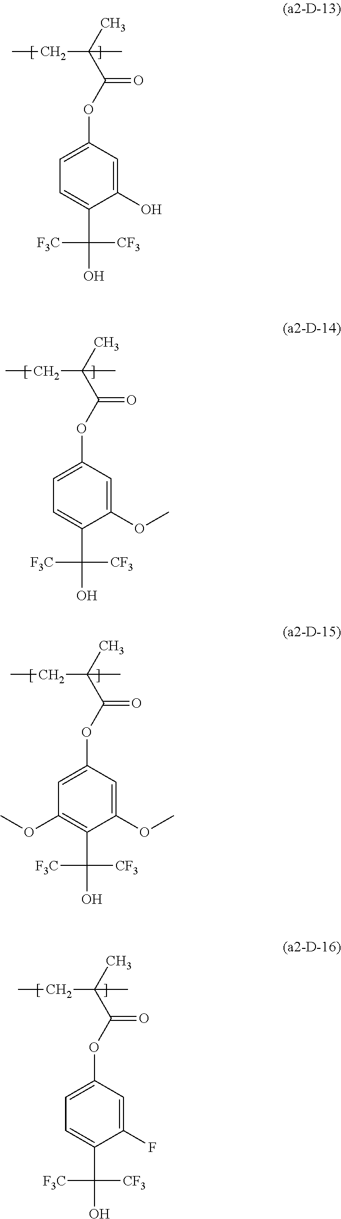

When the resin (A) and/or the like includes the structural unit (a2-D), the content ratio thereof, with respect to all structural units of the resin (A) and/or the like, is preferably 3 mol % or more, more preferably 5 mol % or more, and further preferably 10 mol % or more. In addition, the content ratio is preferably 80 mol % or less, more preferably 75 mol % or less, further preferably 70 mol % or less, and still more preferably 65 mol % or less. Specifically, the content ratio is preferably 3 to 80 mol %, more preferably 5 to 75 mol %, further preferably 10 to 70 mol %, and still more preferably 10 to 65 mol %.

<Structural Unit (a3)>

The lactone ring that the structural unit (a3) has may be a monocyclic ring such as a β-propiolactone ring, a γ-butyrolactone ring or a δ-valerolactone ring, or may be a condensed ring of a monocyclic lactone ring with another ring. Preferable are a γ-butyrolactone ring, an adamantane lactone ring, or a bridged ring including a γ-butyrolactone ring structure (for example, a structural unit represented by the following formula (a3-2)).

The structural unit (a3) is preferably a structural unit represented by formula (a3-1), formula (a3-2), formula (a3-3) or formula (a3-4). One of these may be contained singly, or two or more types may be contained:

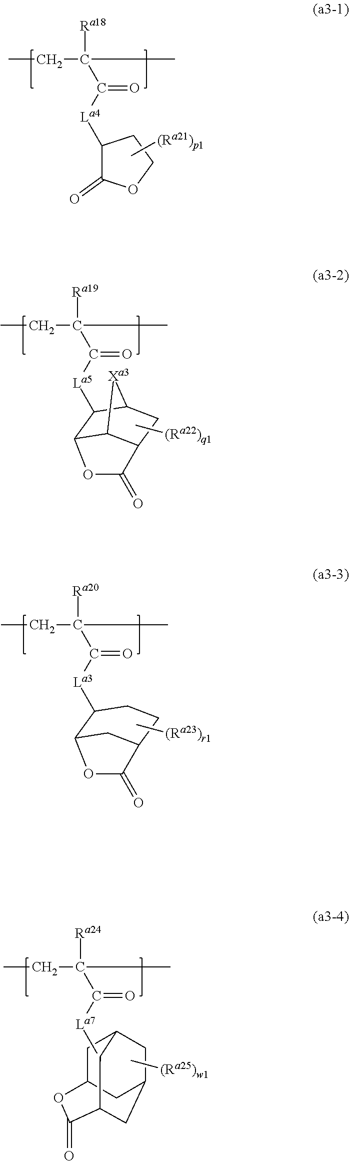

wherein
L^{a4}, L^{a5} and L^{a6} each independently represent —O— or a group represented by *—O—(CH₂)ₖ₃—CO—O—, where k3 represents an integer of 1 to 7;
L^{a7} represents —O—,*—O-L^{a8}-CO—O-L^{a9}-CO—O— or *—O-L^{a8}-O—CO-L^{a9}-O—;
L^{a8} and L^{a9} each independently represent an alkanediyl group having 1 to 6 carbon atoms;
* represents a bonding site to a carbonyl group;
R^{a18}, R^{a19}, R^{a20} and R^{a24} each independently represent a hydrogen atom, a halogen atom or an alkyl group having 1 to 6 carbon atoms and optionally having a halogen atom;
X^{a3} represents —CH₂— or an oxygen atom;
R^{a21} represents an aliphatic hydrocarbon group having 1 to 4 carbon atoms;
R^{a22}, R^{a23} and R^{a25} each independently represent a carboxy group, a cyano group or an aliphatic hydrocarbon group having 1 to 4 carbon atoms;
p1 represents an integer of 0 to 5;
q1 represents an integer of 0 to 3;
r1 represents an integer of 0 to 3;
w1 represents an integer of 0 to 8; and
when p1, q1, r1 and/or w1 are 2 or larger, a plurality of R^{a21}, R^{a22}, R^{a23} and/or R^{a25} may be the same or different from each other.

Examples of the aliphatic hydrocarbon group in R^{a21}, R^{a22}, R^{a23} and R^{a25} include an alkyl group such as a methyl group, an ethyl group, a propyl group, an isopropyl group, a butyl group, a sec-butyl group and a tert-butyl group.

Examples of the halogen atom and the alkyl group optionally having a halogen atom in R^{a18}, R^{a19}, R^{a20} and R^{a24} include the same as those exemplified for R^{a01}, R^{a4} and R^{a5} in formulas (a1-0) to (a1-2).

Examples of the alkanediyl group in L^{a8} and L^{a9} include a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group and a 2-methylbutane-1,4-diyl group.

L^{a4} to L^{a6} are each independently preferably —O— or *—O—(CH₂)ₖ₃—CO—O—, where k3 is an integer of 1 to 4, more preferably —O— or *—O—CH₂—CO—O—, and further preferably an oxygen atom.

L^{a7} is preferably —O— or *—O-L^{a8}-CO—O—, and is more preferably —O—, —O—CH₂—CO—O— or —O—C₂H₄—CO—O—.

R^{a18}, R^{a19}, R^{a20} and R^{a24} are preferably hydrogen atoms or alkyl groups having 1 to 4 carbon atoms, are more preferably hydrogen atoms, methyl groups, or ethyl groups, and are further preferably hydrogen atoms or methyl groups.

R^{a21} is preferably a methyl group.

R^{a22}, R^{a23} and R^{a25} are each independently preferably a carboxy group, a cyano group or a methyl group.

p1, q1 and r1 are each independently preferably an integer of 0 to 2, and more preferably 0 or 1.

w1 is preferably an integer of 0 to 2, and is more preferably 0 or 1.

In particular, formula (a3-4) is preferably formula (a3-4′):

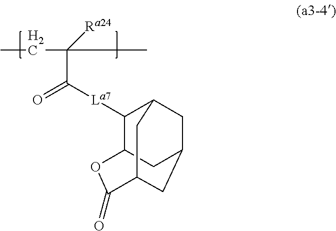

wherein R^{a24} and L^{a7} have the same meanings as described above.

Examples of the structural unit (a3) include structural units derived from a monomer disclosed in Japanese Patent Laid-Open No. 2010-204646, a monomer disclosed in Japanese Patent Laid-Open No. 2000-122294, and a monomer disclosed in Japanese Patent Laid-Open No. 2012-41274. As the structural unit (a3), preferable is a structural unit represented by any one of formulas (a3-1-1), (a3-1-2), (a3-2-1), (a3-2-2), (a3-3-1), (a3-3-2), and (a3-4-1) to (a3-4-12), as well as structural units in which the methyl groups corresponding to R^{a18}, R^{a19}, R^{a20} and R^{a24} in formulas (a3-1) to (a3-4) are replaced with hydrogen atoms.

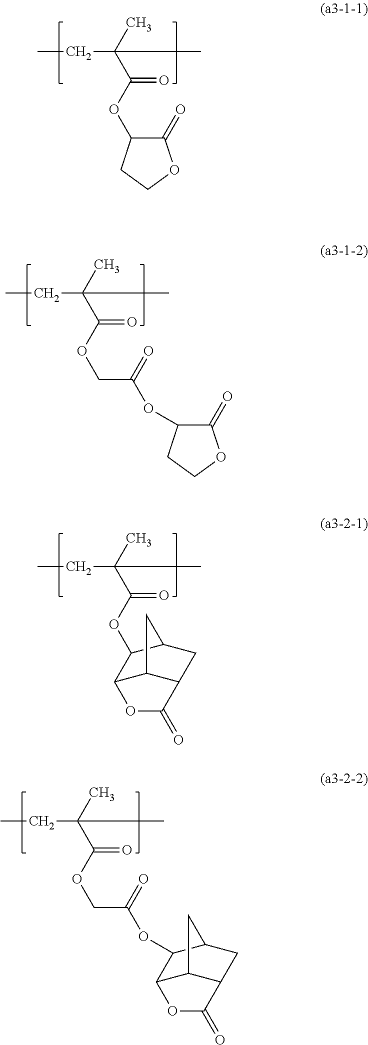

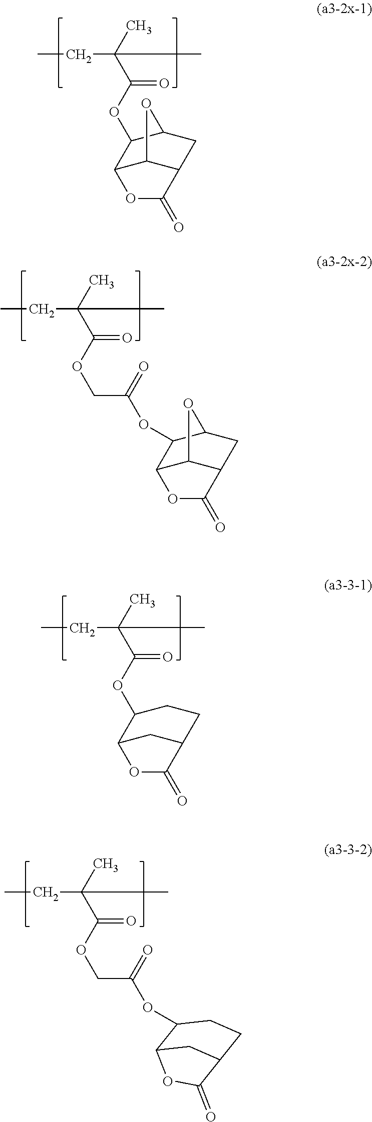

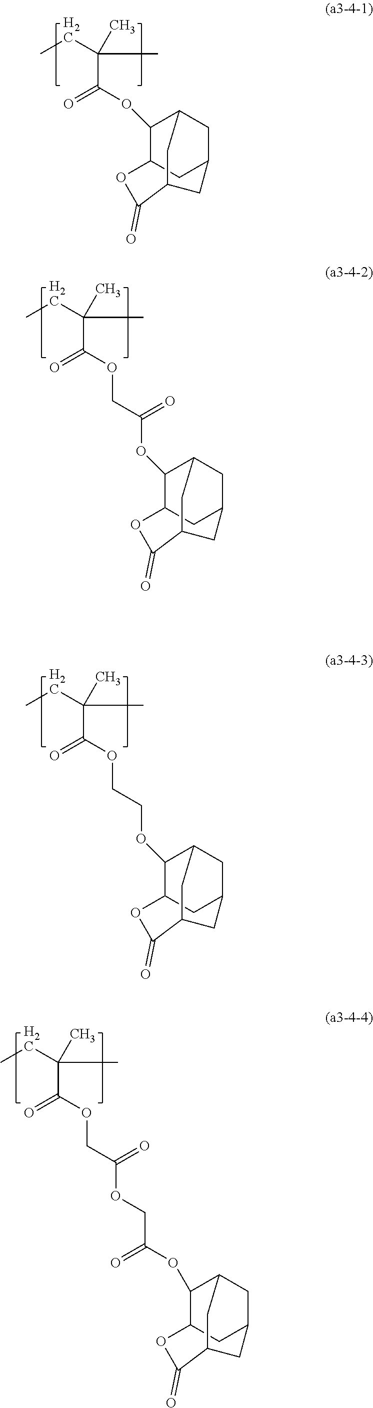

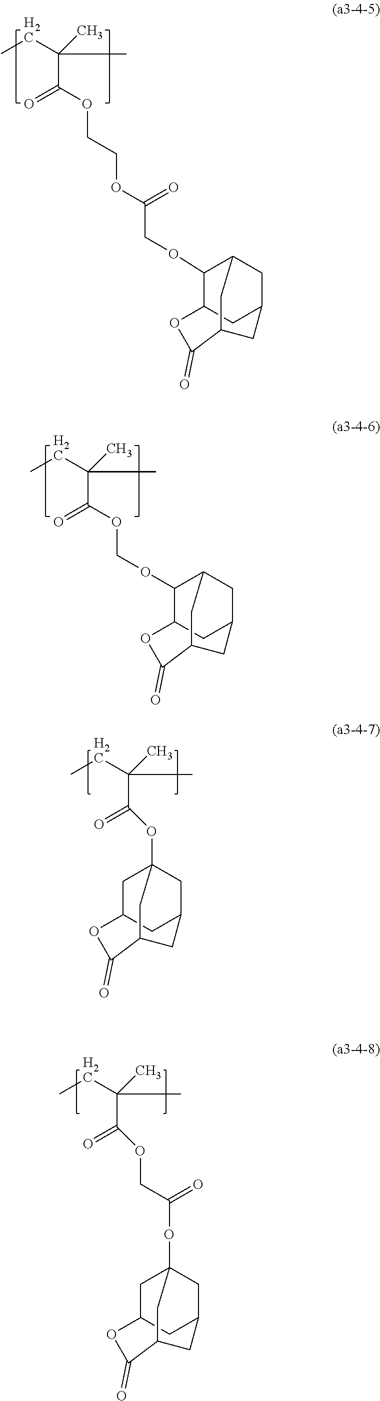

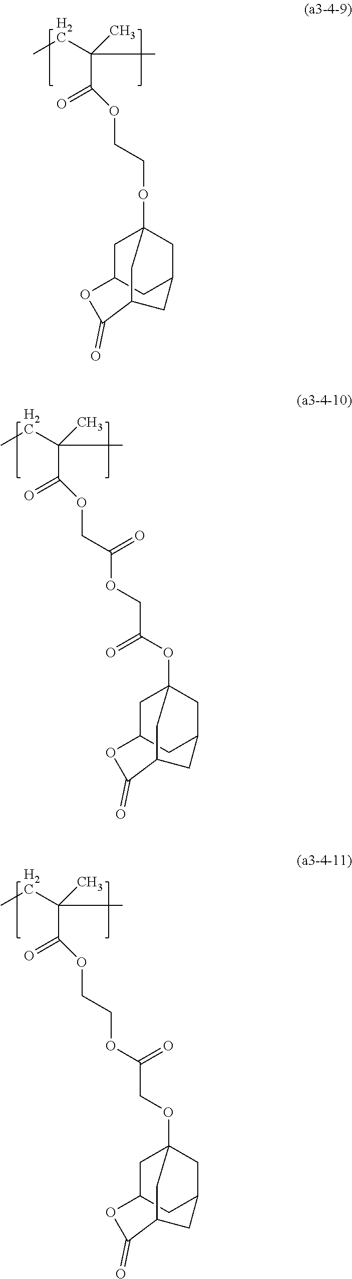

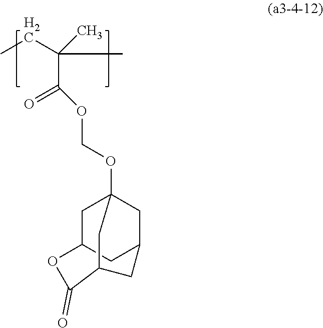

When the resin (A) and/or the like includes the structural unit (a3), the total content ratio thereof, with respect to all structural units of the resin (A) and/or the like, is, for example, 1 mol % or more, preferably 3 mol % or more, more preferably 5 mol % or more, and further preferably 10 mol % or more. In addition, the total content ratio is, for example, 70 mol % or less, preferably 65 mol % or less, more preferably 60 mol % or less, further preferably 50 mol % or less, still more preferably 40 mol % or less, still further preferably 30 mol % or less, further more preferably 25 mol % or less, and still even more preferably 20 mol % or less. Specifically, the total content ratio is, for example, 1 to 70 mol %, preferably 3 to 70 mol %, more preferably 3 to 65 mol %, further preferably 5 to 65 mol %, still more preferably 5 to 60 mol %, and still further preferably 10 to 60 mol %.

In addition, the respective content ratios of the structural unit (a3-1), the structural unit (a3-2), the structural unit (a3-3) and the structural unit (a3-4) are preferably 1 mol % or more, more preferably 3 mol % or more, further preferably 5 mol % or more, and further more preferably 10 mol % or more, with respect to all structural units of the resin (A) and/or the like. In addition, the content ratios are preferably 60 mol % or less, more preferably 55 mol % or less, further preferably 50 mol % or less, still more preferably 40 mol % or less, still further preferably 30 mol % or less, further more preferably 25 mol % or less, and still even more preferably 20 mol % or less. Specifically, the content ratios are preferably 1 to 60 mol %, more preferably 3 to 60 mol %, further preferably 3 to 55 mol %, further more preferably 5 to 55 mol %, still more preferably 5 to 50 mol %, and still further preferably 10 to 50 mol %.

<Structural Unit (a4)>

The structural unit represented by formula (a4) is shown below:

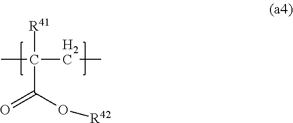

wherein
R⁴¹ represents a hydrogen atom or a methyl group; and
R⁴² represents an aliphatic hydrocarbon group having 1 to 48 carbon atoms and a halogen atom, and —CH₂— contained in the aliphatic hydrocarbon group is optionally replaced with —O— or —CO—.

Examples of the aliphatic hydrocarbon group represented by R⁴² include a straight-chain or branched chain hydrocarbon group, a monocyclic or polycyclic alicyclic hydrocarbon group, and a group formed by combining these groups.

Examples of the chain hydrocarbon group include a straight-chain alkyl group such as a methyl group, an ethyl group, a propyl group, a butyl group, a pentyl group, a hexyl group, a heptyl group, an octyl group, a decyl group, a dodecyl group, a pentadecyl group, a hexadecyl group, a heptadecyl group and an octadecyl group, and a branched alkyl group such as an isopropyl group and an isobutyl group.

Examples of the monocyclic or polycyclic alicyclic hydrocarbon group include: a monocyclic alicyclic hydrocarbon group that is a monocyclic cycloalkyl group such as a cyclopentyl group, a cyclohexyl group, a cycloheptyl group and a cyclooctyl group; and a polycyclic alicyclic hydrocarbon group that is a polycyclic cycloalkyl group such as a decahydronaphthyl group, an adamantly group, a norbornyl group and the following groups, where * represents a bonding site.

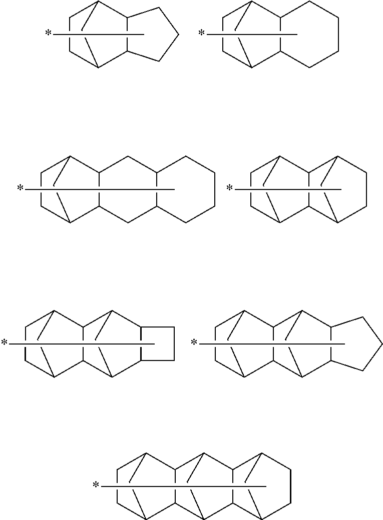

Examples of the group formed by combination include a group formed by combining one or more alkyl groups or one or more alkanediyl groups with one or more alicyclic hydrocarbon groups, such as -alkanediyl group-alicyclic hydrocarbon group, -alicyclic hydrocarbon group-alkyl group and -alkanediyl group-alicyclic hydrocarbon group-alkyl group.

Examples of the alkanediyl group include: a straight-chain alkanediyl group such as a methylene group, an ethylene group, a propane-1,3-diyl group, a butane-1,4-diyl group, a pentane-1,5-diyl group and a hexane-1,6-diyl group; and a branched alkanediyl group such as a propane-1,2-diyl group, a butane-1,3-diyl group, a 2-methylpropane-1,2-diyl group, a 1-methylbutane-1,4-diyl group and a 2-methylbutane-1,4-diyl group. The terminal of the branched alkanediyl group may be a methyl group.

Examples of the halogen atom that the aliphatic hydrocarbon group included in R⁴² has include a fluorine atom, a chlorine atom, a bromine atom and an iodine atom.

Examples of the group formed by replacing —CH₂— in the aliphatic hydrocarbon group included in R⁴² with —O— or —CO— include a hydroxy group, a carboxy group, a carbonyl group, an oxy group, an alkoxy group, an alkoxycarbonyl group, an alkylcarbonyl group, an alkylcarbonyloxy group, an alkanediyloxy group, an alkanediyloxycarbonyl group, an alkanediylcarbonyl group, an alkanediylcarbonyloxy group, a cycloalkoxy group, a cycloalkylalkoxy group and a combined group of two or more of these groups. Examples of these groups formed by the replacement include the same groups as those exemplified in the present specification, within the range permitted by the upper limit of the number of carbon atoms.

As the structural unit (a4), R⁴² is preferably an aliphatic hydrocarbon group having a fluorine atom, and examples of the structural unit (a4) in which R⁴² is an aliphatic hydrocarbon group having a fluorine atom include a structural unit represented by formula (a4-1) (hereinafter referred to as “structural unit (a4-1)” in some cases), a structural unit represented by formula (a4-2) (hereinafter referred to as “structural unit (a4-2)” in some cases), a structural unit represented by formula (a4-3) (hereinafter referred to as “structural unit (a4-3)” in some cases) and a structural unit represented by formula (a4-A) (hereinafter referred to as “structural unit (a4-A)” in some cases).

The structural unit represented by formula (a4-1) is a structural unit shown below:

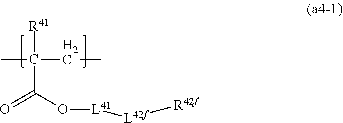

wherein
R⁴¹ represents a hydrogen atom or a methyl group;
L⁴¹ represents a single bond or an alkanediyl group having 1 to 4 carbon atoms;
L^{42f} represents an alkanediyl group having 1 to 8 carbon atoms and a fluorine atom, or a cycloalkanediyl group having 3 to 12 carbon atoms and a fluorine atom; and
R^{42f} represents a hydrogen atom or a fluorine atom.

Examples of the alkanediyl group in L⁴¹ include a straight-chain alkanediyl group such as a methylene group, an ethylene group, a propane-1,3-diyl group and a butane-1,4-diyl group, and a branched alkanediyl group such as an ethane-1,1-diyl group, a propane-1,2-diyl group, a butane-1,3-diyl group, a 2-methylpropane-1,3-diyl group and a 2-methylpropane-1,2-diyl group. The terminal of the branched alkanediyl group may be a methyl group.

Examples of the alkanediyl group having a fluorine atom in L^{42f} include: a straight-chain alkanediyl group having a fluorine atom such as a methylene group having a fluorine atom, an ethylene group having a fluorine atom, a propane-1,3-diyl group having a fluorine atom, a butane-1,4-diyl group having a fluorine atom, a pentane-1,5-diyl group having a fluorine atom, a hexane-1,6-diyl group having a fluorine atom, a heptane-1,7-diyl group having a fluorine atom and an octane-1,8-diyl group having a fluorine atom; and
a branched alkanediyl group having a fluorine atom such as an ethane-1,1-diyl group having a fluorine atom, a propane-1,1-diyl group having a fluorine atom, a propane-1,2-diyl group having a fluorine atom, a propane-2,2-diyl group having a fluorine atom, a pentane-2,4-diyl group having a fluorine atom, a 2-methylpropane-1,3-diyl group having a fluorine atom, a 2-methylpropane-1,2-diyl group having a fluorine atom, a pentane-1,4-diyl group having a fluorine atom and a 2-methylbutane-1,4-diyl group having a fluorine atom. The terminal of the branched alkanediyl group may be a methyl group optionally having a fluorine atom.

Examples of the cycloalkanediyl group having a fluorine atom in L^{42f} include: a monocyclic cycloalkanediyl group having a fluorine atom such as a cyclobutane-1,3-diyl group having a fluorine atom, a cyclopentane-1,3-diyl group having a fluorine atom, a cyclohexane-1,4-diyl group having a fluorine atom, a cyclohexene-3,6-diyl group having a fluorine atom, a cycloheptane-1,4-diyl group having a fluorine atom and a cyclooctane-1,5-diyl group having a fluorine atom; and
a polycyclic cycloalkanediyl group having a fluorine atom such as a norbornane-1,4-diyl group having a fluorine atom, a norbornane-2,5-diyl group having a fluorine atom, a 5-norbornene-2,3-diyl group having a fluorine atom, an adamantane-1,5-diyl group having a fluorine atom and an adamantane-2,6-diyl group having a fluorine atom.

The number of fluorine atoms that the alkanediyl group and cycloalkanediyl group having a fluorine atom in L^{42f} have may be 1 or more, and is preferably 2 or more.

The alkanediyl group and cycloalkanediyl group having a fluorine atom in L^{42f} are preferably a perfluoroalkanediyl group and a perfluorocycloalkanediyl group, respectively.

Examples of the perfluoroalkanediyl group in L^{42f} include a difluoromethylene group, a perfluoroethylene group, a perfluoroethylfluoromethylene group, a perfluoropropane-1,3-diyl group, a perfluoropropane-1,2-diyl group, a perfluoropropane-2,2-diyl group, a perfluorobutane-1,4-diyl group, a perfluorobutane-2,2-diyl group, a perfluorobutane-1,2-diyl group, a perfluoropentane-1,5-diyl group, a perfluoropentane-2,2-diyl group, a perfluoropentane-3,3-diyl group, a perfluorohexane-1,6-diyl group, a perfluorohexane-2,2-diyl group, a perfluorohexane-3,3-diyl group, a perfluoroheptane-1,7-diyl group, a perfluoroheptane-2,2-diyl group, a perfluoroheptane-3,4-diyl group, a perfluoroheptane-4,4-diyl group, a perfluorooctane-1,8-diyl group, a perfluorooctane-2,2-diyl group, a perfluorooctane-3,3-diyl group and a perfluorooctane-4,4-diyl group.

Examples of the perfluorocycloalkanediyl group in L^{42f} include a perfluorocyclohexanediyl group, a perfluorocyclopentanediyl group, a perfluorocycloheptanediyl group and a perfluoroadamantanediyl group.

L⁴¹ is preferably a single bond or an alkanediyl group having 1 to 3 carbon atoms, is more preferably a single bond, a methylene group or an ethylene group, and is further preferably a single bond or a methylene group.

L^{42f} is preferably a perfluoroalkanediyl group having 1 to 8 carbon atoms or a perfluorocycloalkanediyl group having 3 to 12 carbon atoms, is more preferably a perfluoroalkanediyl group having 1 to 6 carbon atoms, and is further preferably a perfluoroalkanediyl group having 1 to 3 carbon atoms.

Examples of the structural unit (a4-1) include the structural units shown below and the structural units in which the methyl group corresponding to R⁴¹ in the structural unit (a4-1) in the following structural units is replaced with a hydrogen atom.

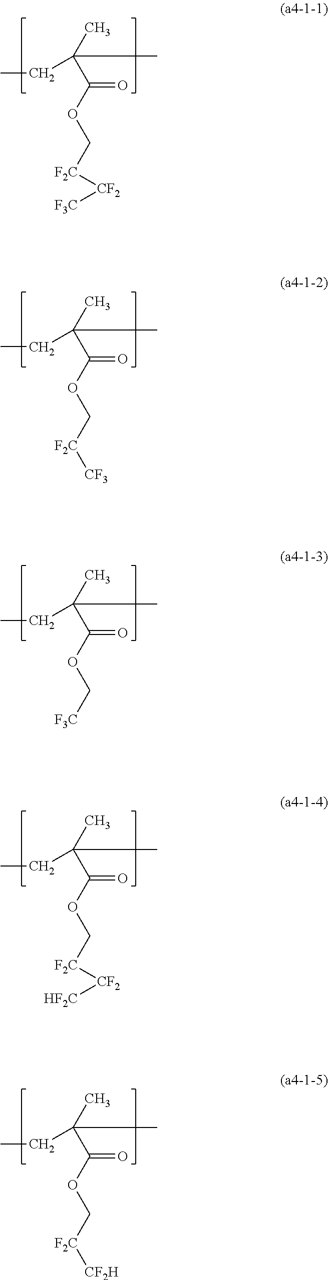

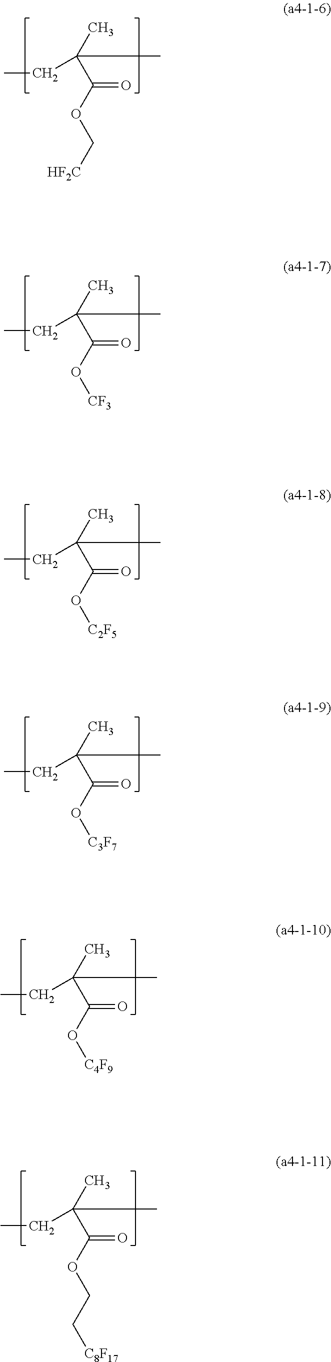

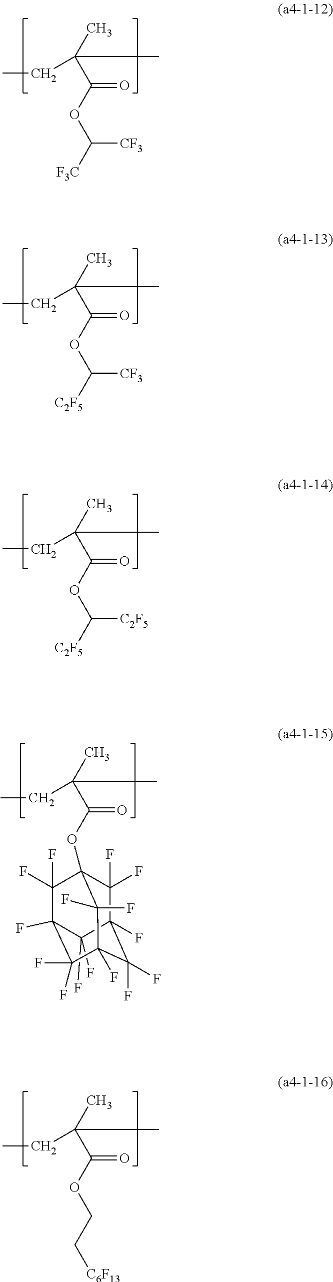

The structural unit represented by formula (a4-2) is a structural unit shown below:

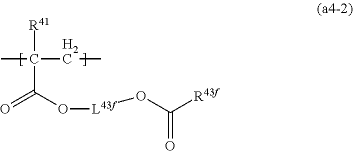

wherein
R⁴¹ represents a hydrogen atom or a methyl group;
L^{43f} represents an aliphatic hydrocarbon group having 1 to 20 carbon atoms and optionally having a fluorine atom, and —CH₂— contained in the aliphatic hydrocarbon group is optionally replaced with —O— or —CO—; and
R^{43f} represents an aliphatic hydrocarbon group having 1 to 20 carbon atoms and optionally having a fluorine atom, and —CH₂— contained in the aliphatic hydrocarbon group is optionally replaced with —O— or —CO—,
provided that at least one of L^{43f} and R^{43f} has a fluorine atom, and the upper limit of the total number of carbon atoms of L^{43f} and R^{43f} is 21.

Examples of the aliphatic hydrocarbon group in L^{43f} and R^{43f} include a straight-chain or branched chain hydrocarbon group and a monocyclic or polycyclic alicyclic hydrocarbon group, as well as a group formed by combining these groups. Examples of the aliphatic hydrocarbon group in L^{43f} and R^{43f} include the same aliphatic hydrocarbon groups as those exemplified as the aliphatic hydrocarbon group of R⁴² in formula (a4), within the range permitted by the upper limit of the total number of carbon atoms of L^{43f} and R^{43f}.

As the aliphatic hydrocarbon group of L^{43f} an alkanediyl group having 1 to 6 carbon atoms or a group represented by formula (L43f-1) is preferable:

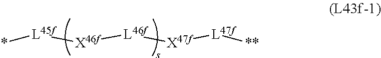

wherein
s represents 0 or 1;
L^{45f} and L^{46f} each independently represent a divalent aliphatic hydrocarbon group having 1 to 5 carbon atoms and optionally having a fluorine atom;
L^{47f} represents a single bond or a divalent aliphatic hydrocarbon group having 1 to 5 carbon atoms and optionally having a fluorine atom;
X^{46f} and X^{47f} each independently represent —O—, —CO—, —CO—O— or —O—CO—,
provided that the total number of carbon atoms of L^{45f}, L^{46f}, L^{47f}, X^{46f} and X^{47f} is 7 or less; and
* and ** represent bonding sites, and ** represents a bonding site to —O—CO—R^{43f}.

Examples of the divalent aliphatic hydrocarbon group represented by L^{45f}, L^{46f} and L^{47f} in the group represented by formula (L43f-1) include: a straight-chain or branched alkanediyl group and a monocyclic or polycyclic divalent alicyclic hydrocarbon group, as well as a divalent aliphatic hydrocarbon group formed by combining an alkanediyl group and a divalent alicyclic hydrocarbon group. Specific examples thereof include a methylene group, an ethylene group, a propane-1,3-diyl group, a propane-1,2-diyl group, a butane-1,4-diyl group, a 1-methylpropane-1,3-diyl group, a 2-methylpropane-1,3-diyl group and a 2-methylpropane-1,2-diyl group.
s is preferably 0.

Examples of the group represented by formula (L43f-1) include the following groups. In the following examples of the groups, * and ** each represent a bonding site, and ** is a bonding site to —O—CO—R^{43f}.

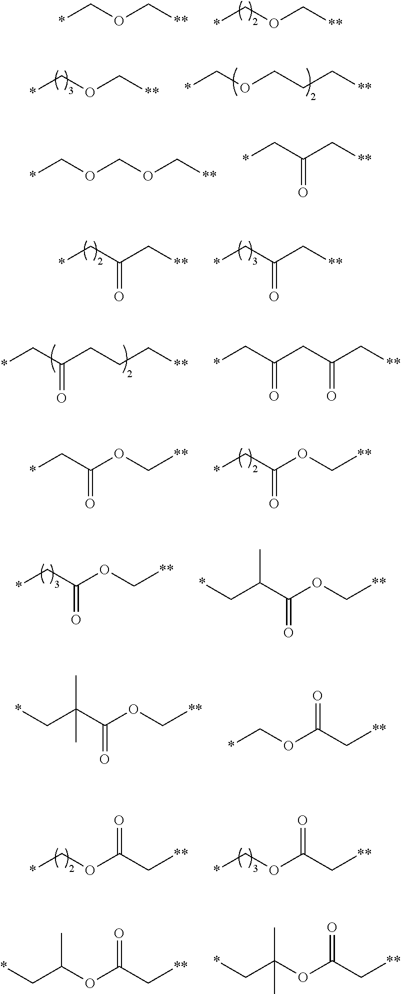

R^{43f} is preferably an aliphatic hydrocarbon group having a fluorine atom.

In this case, examples of the structural unit represented by formula (a4-2) include the structural unit represented by formula (a4-2A) or (a4-2B):

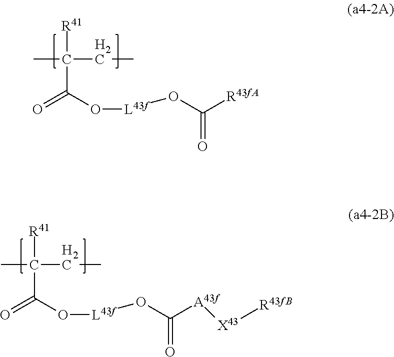

wherein
R⁴¹ and L^{43f} represent the same as those in formula (a4-2);
R^{43fA} represents an aliphatic hydrocarbon group having 1 to 20 carbon atoms and a fluorine atom;
A^{43f} represents a divalent aliphatic hydrocarbon group having 1 to 17 carbon atoms and optionally having a fluorine atom, and —CH₂— contained in the aliphatic hydrocarbon group is optionally replaced with —O— or —CO—;
X⁴³ represents **—O—CO— or **—CO—O—, and ** represents a bonding site to A^{43f}; and
R^{43fB} represents an aliphatic hydrocarbon group having 1 to 17 carbon atoms and optionally having a fluorine atom,
provided that in formula (a4-2A), the total number of carbon atoms of L^{43f} and R^{43fA} is 21 or less, and in formula (a4-2B), the total number of carbon atoms of L^{43f}, A^{43f}, X⁴³ and R^{43fB} is 21 or less, and at least one of A^{43f} and R^{43fB} has at least one fluorine atom.

Examples of the aliphatic hydrocarbon group of R^{43fA} include the same aliphatic hydrocarbon group as those exemplified for R⁴², within the range permitted by the upper limit of the number of carbon atoms.

R^{43fA} is preferably an alkyl group having 1 to 13 carbon atoms and a fluorine atom, a cycloalkyl group having 3 to 12 carbon atoms and a fluorine atom, or a combined group of these groups, and is more preferably *—(CF₂)_{n43f}—R^{42′}, where * represents a bonding site to a carbonyl group, n43f represents an integer of 1 to 6, and R^{42′} represents a hydrogen atom or a fluorine atom, or a perfluorocycloalkyl group having 3 to 12 carbon atoms.

Examples of the aliphatic hydrocarbon group of A^{43f} and R^{43fB} include the same aliphatic hydrocarbon groups as those exemplified for R⁴² in formula (a4), within the range permitted by the upper limit of the number of carbon atoms.

A^{43f} is preferably a divalent chain hydrocarbon group optionally having a fluorine atom, a divalent alicyclic hydrocarbon group or a combined group of these groups, is more preferably a divalent chain hydrocarbon group having a fluorine atom, and is further preferably a fluorinated alkanediyl group having 1 to 6 carbon atoms.

The aliphatic hydrocarbon group optionally having a fluorine atom of R^{43fB} is preferably a chain hydrocarbon group, an alicyclic hydrocarbon group or a combined group of these groups, optionally having a fluorine atom, is more preferably an alkyl group having 1 to 12 carbon atoms, a cycloalkyl group having 3 to 12 carbon atoms or a combined group of these groups, optionally having a fluorine atom, and is further preferably a fluorinated alkyl group such as a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a heptyl group, a perfluoroheptyl group, an octyl group or a perfluorooctyl group, a cyclopropylmethyl group, a cyclopropyl group, a cyclobutylmethyl group, a cyclopentyl group, a cyclohexyl group, a perfluorocyclohexyl group, an adamantyl group, an adamantylmethyl group, an adamantyldimethyl group, a norbornyl group, a norbornylmethyl group, a perfluoroadamantyl group or a perfluoroadamantylmethyl group.

In formula (a4-2B), examples of the structure that the group represented by *-A^{43f}-X⁴³—R^{43fB} can take include the following structures, where * is a bonding site to a carbonyl group.

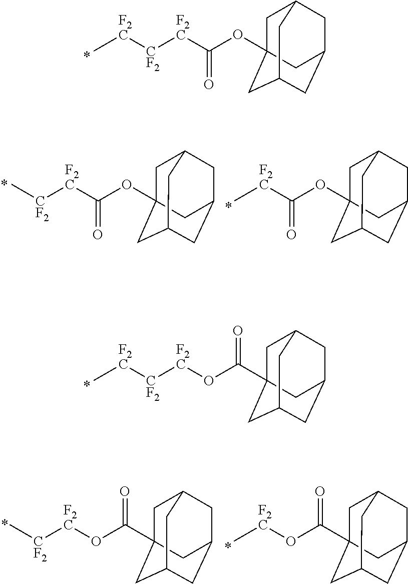

Examples of the structural unit represented by formula (a4-2A) include the structural units shown below and the structural units in which the methyl group corresponding to R⁴¹ in the structural unit represented by formula (a4-2A) in the following structural units is replaced with a hydrogen atom.

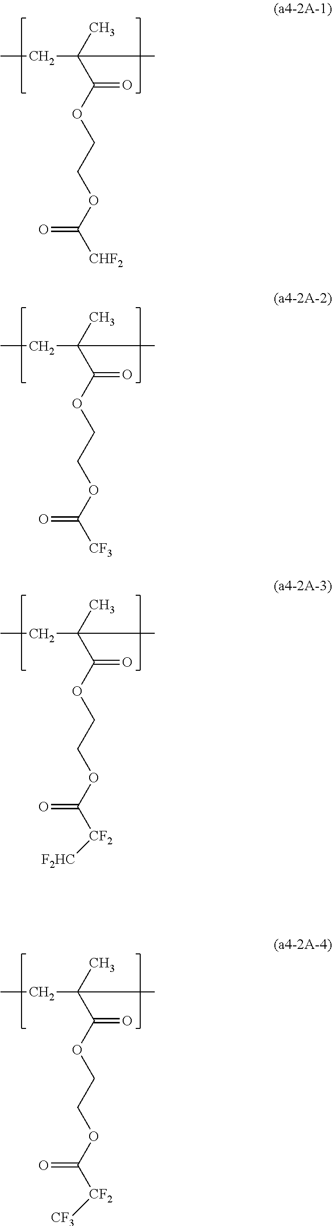

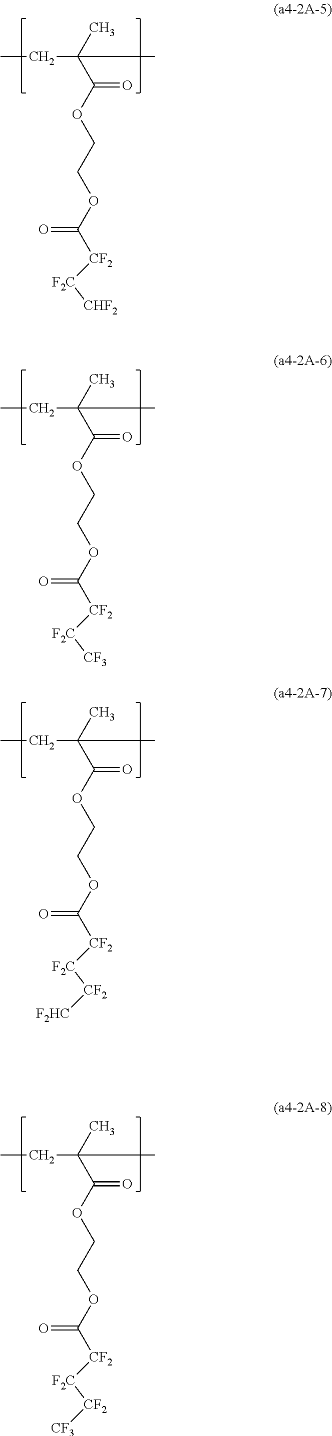

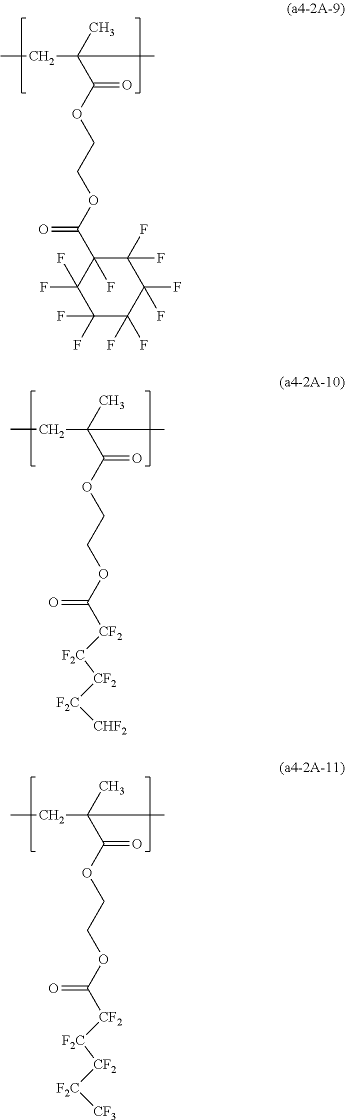

Examples of the structural unit represented by formula (a4-2B) include the structural units shown below and the structural units in which the methyl group corresponding to R⁴¹ in the structural unit represented by formula (a4-2B) in the following structural units is replaced with a hydrogen atom.

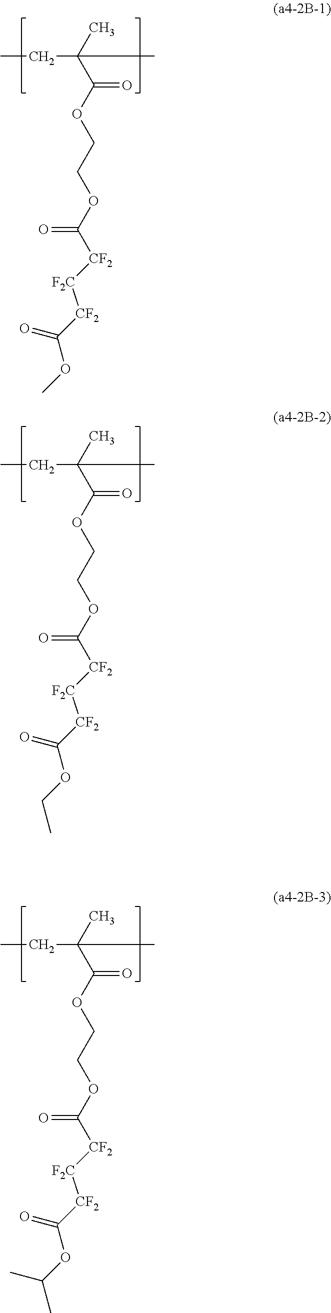

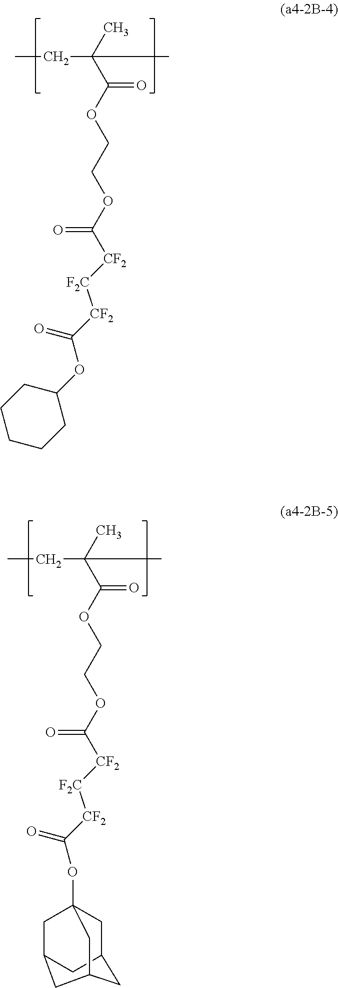

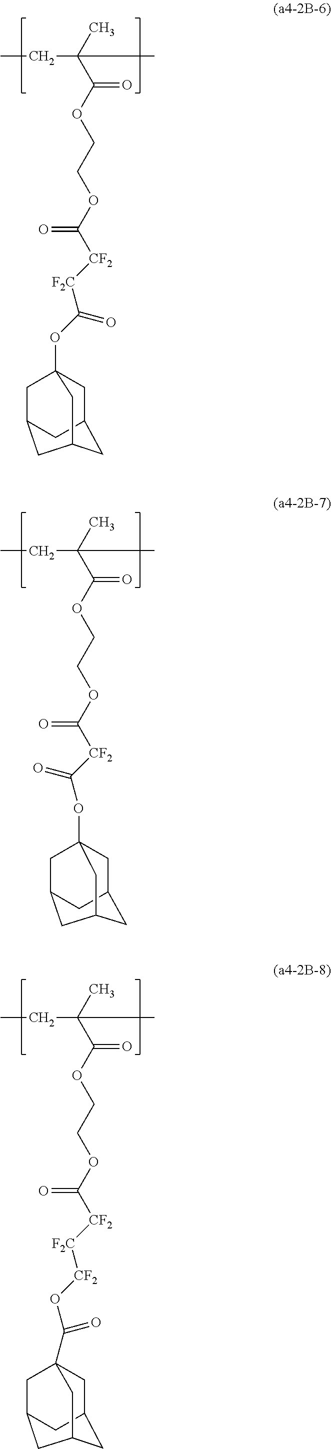

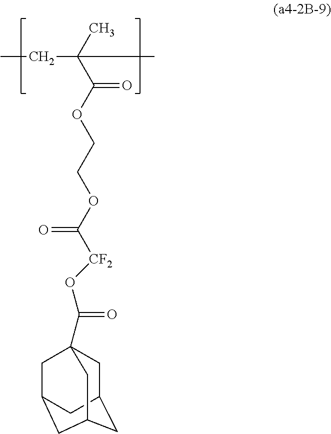

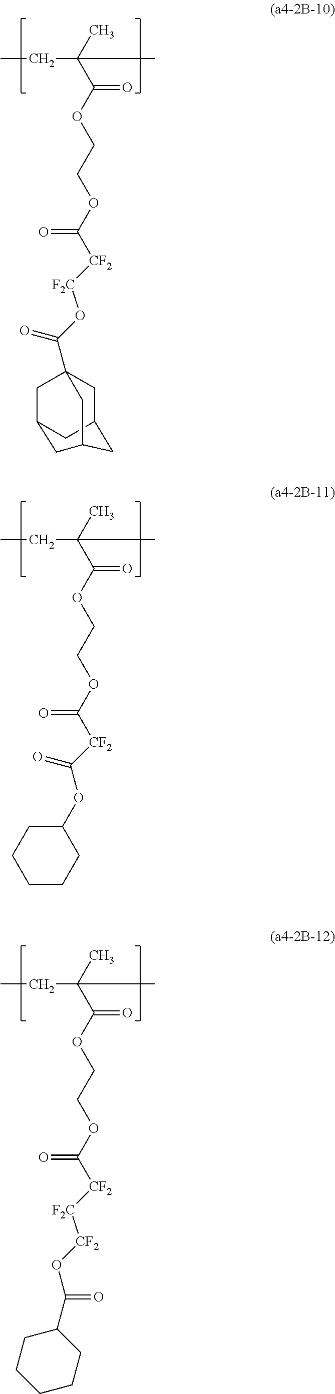

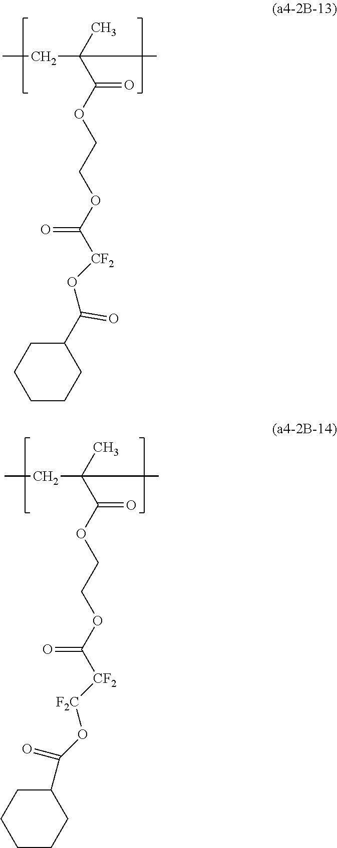

Examples of the structural unit (a4) also include a structural unit represented by formula (a4-3):

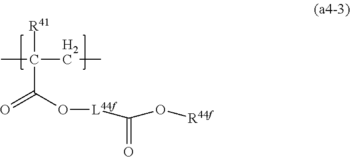

wherein
R⁴¹ represents a hydrogen atom or a methyl group;
L^{44f} represents an aliphatic hydrocarbon group having 1 to 20 carbon atoms and optionally having a fluorine atom, and —CH₂— contained in the aliphatic hydrocarbon group is optionally replaced with —O— or —CO—; and
R^{44f} represents an aliphatic hydrocarbon group having 1 to 20 carbon atoms and optionally having a fluorine atom, and —CH₂— contained in the aliphatic hydrocarbon group is optionally replaced with —O— or —CO—,
provided that at least one of L^{44f} and R^{44f} has a fluorine atom, and the upper limit of the total number of carbon atoms of L⁴⁴ and R^{44f} is 21.

Examples of the aliphatic hydrocarbon group in L^{44f} and R^{44f} include a straight-chain or branched chain hydrocarbon group and a monocyclic or polycyclic alicyclic hydrocarbon group, as well as a group formed by combining these groups. Examples of the aliphatic hydrocarbon group in L^{44f} and R^{44f} include the same aliphatic hydrocarbon groups as those exemplified as the aliphatic hydrocarbon group of R⁴² in formula (a4), within the range permitted by the upper limit of the total number of carbon atoms of L^{44f} and R^{44f}.

L^{44f} is preferably an alkanediyl group having 1 to 14 carbon atoms, where —CH₂— contained in the alkanediyl group is optionally replaced with —O— or —CO—, and is more preferably a group represented by —(CH₂)ⱼ₁—, —(CH₂)ⱼ₂—O—(CH₂)ⱼ₃— or —(CH₂)ⱼ₄—CO—O—(CH₂)ⱼ₅—, where j1 to j5 each independently represent an integer of 1 to 6. In addition, also further preferable is an alkanediyl group having 1 to 4 carbon atoms, where one —CH₂— contained in the alkanediyl group is optionally replaced with —O—, and one —CH₂—CH₂— contained in the alkanediyl group is optionally replaced with —CO—O— or —O—CO—.

R^{44f} is preferably an aliphatic hydrocarbon group having 1 to 10 carbon atoms and a fluorine atom, is more preferably an alkyl group having 1 to 10 carbon atoms and a fluorine atom, an alicyclic hydrocarbon group having 3 to 10 carbon atoms and a fluorine atom, or a combined group of these groups, is further preferably an alkyl group having 1 to 10 carbon atoms and a fluorine atom, and is even more preferably an alkyl group having 1 to 6 carbon atoms and a fluorine atom.

Examples of the structural unit represented by formula (a4-3) include the following structural units and the structural units in which the methyl group corresponding to R⁴¹ in the structural unit (a4-3) in the structural units represented by the following formulas is replaced with a hydrogen atom.

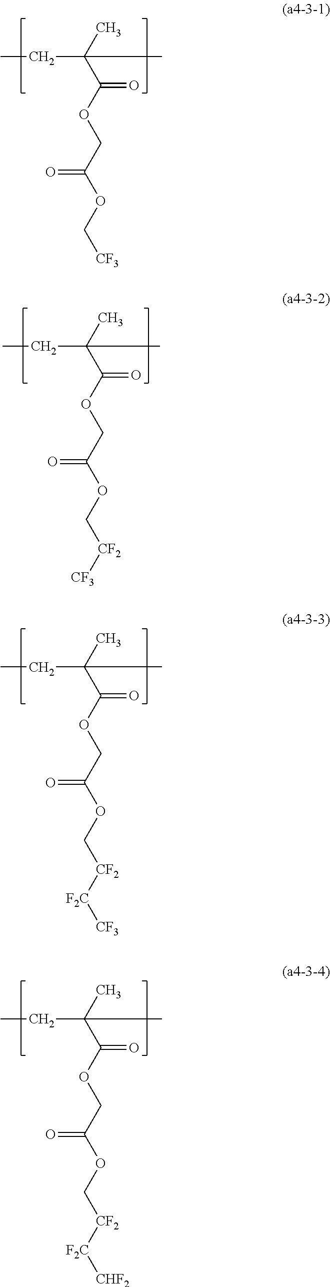

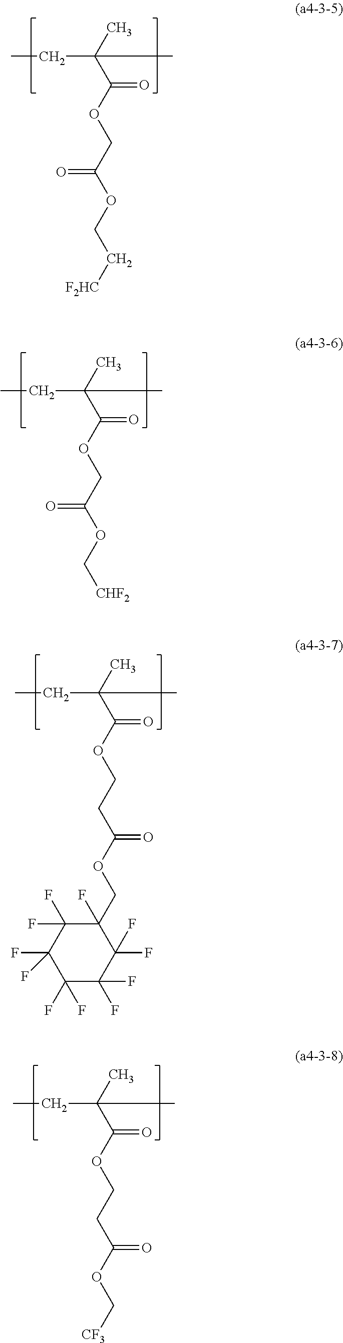

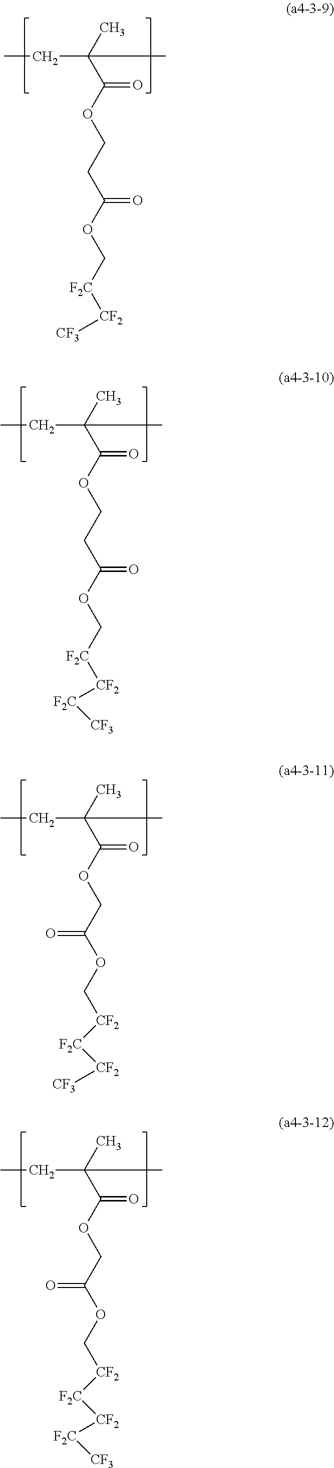

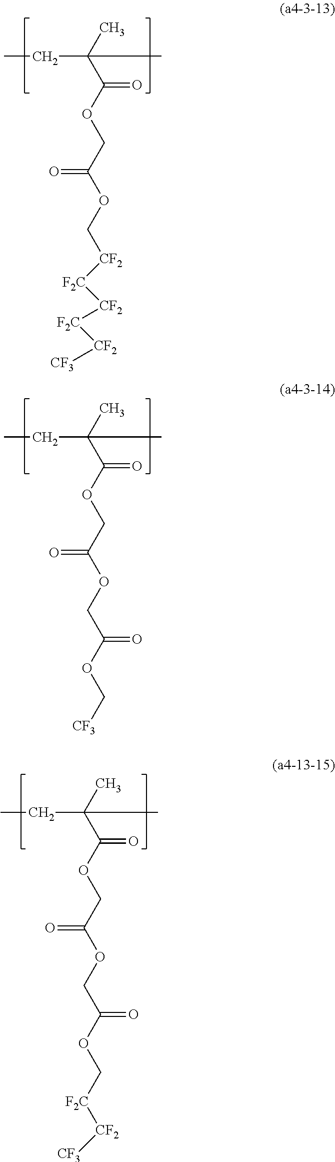

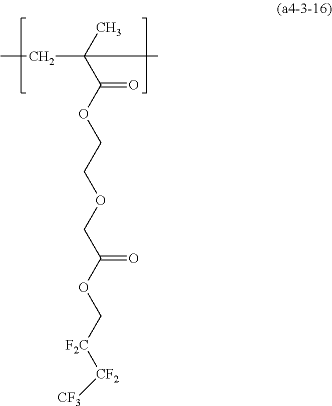

Examples of the structural unit (a4) also include a structural unit represented by formula (a4-A):

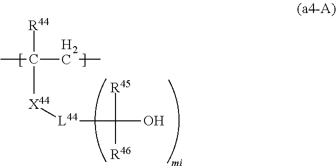

wherein
R⁴⁴ represents a hydrogen atom, a halogen atom or an alkyl group having 1 to 6 carbon atoms and optionally having a halogen atom;
X⁴⁴ represents a single bond or *—CO—O—, and* represents a bonding site to a carbon atom to which R⁴⁴ is bonded;
L⁴⁴ represents an aliphatic hydrocarbon group having 1 to 28 carbon atoms and optionally having a substituent, and —CH₂— contained in the aliphatic hydrocarbon group is optionally replaced with —O—, —S—, —SO₂—, —SO— or —CO—;
R⁴⁵ represents a fluorinated alkyl group having 1 to 8 carbon atoms;
R⁴⁶ represents a hydrogen atom or a fluorinated alkyl group having 1 to 6 carbon atoms, which may be bonded to L⁴⁴ to form an alicyclic hydrocarbon group having 3 to 12 carbon atoms, and —CH₂— contained in the alicyclic hydrocarbon group is optionally replaced with —O—; and
mi represents an integer of 1 to 4, and when mi is 2 or larger, a plurality of groups in parentheses may be the same or different from each other.

Examples of the halogen atom and the alkyl group optionally having a halogen atom in R⁴⁴ include the same as those exemplified for R^{a01}, R^{a4} and R^{a5} in formulas (a1-0) to (a1-2).

R⁴⁴ is preferably a hydrogen atom or an alkyl group having 1 to 4 carbon atoms, is more preferably a hydrogen atom or an alkyl group having 1 to 3 carbon atoms, is further preferably a hydrogen atom, a methyl group or an ethyl group, and is further more preferably a hydrogen atom or a methyl group.

Examples of the aliphatic hydrocarbon group in L⁴⁴ include a chain hydrocarbon group, and a monocyclic or polycyclic (including a spiro ring, a condensed ring or a bridged ring) alicyclic hydrocarbon group, and it may be a combined group of two or more of these groups (for example, a hydrocarbon group formed from an alicyclic hydrocarbon group and a chain hydrocarbon group).

Examples of the chain hydrocarbon group include a divalent to pentavalent chain hydrocarbon group such as an alkanediyl group, an alkanetriyl group, an alkanetetrayl group and an alkanepentayl group.

Examples of the alkanediyl group include: a straight-chain alkanediyl group such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group; and
a branched alkanediyl group such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group.

In addition, examples thereof also include a group in which one or more hydrogen atoms of the above groups are replaced with bonding sites.

The number of carbon atoms of the chain hydrocarbon group is preferably 1 to 18, is more preferably 1 to 12, is further preferably 1 to 10, is still more preferably 1 to 9, is still further preferably 1 to 8, is further more preferably 1 to 6, is still even more preferably 1 to 5, and is particularly preferably 1 to 4.

Examples of the monocyclic or polycyclic alicyclic hydrocarbon group include the following alicyclic hydrocarbon groups. The bonding site can be set to any position.

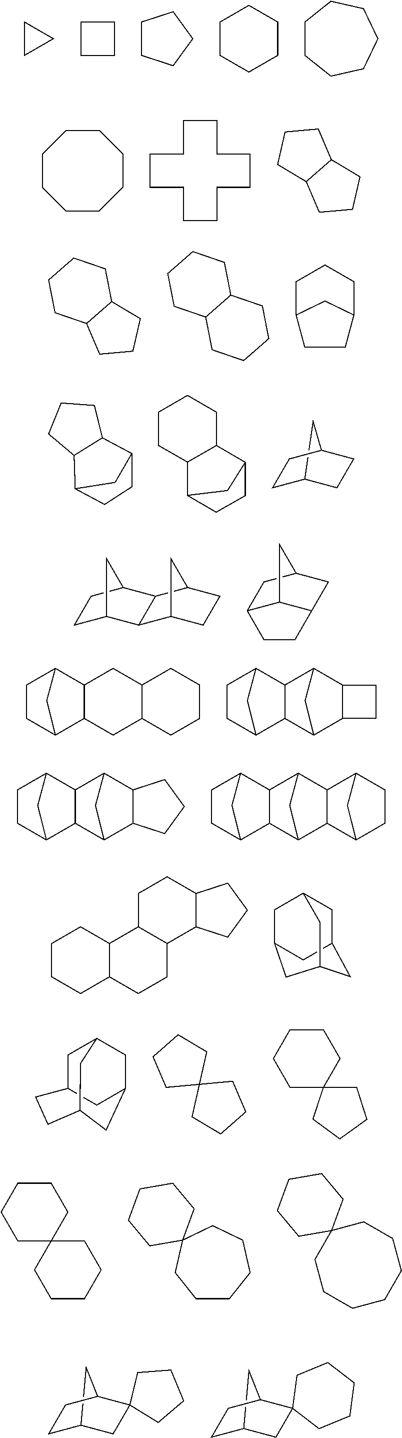

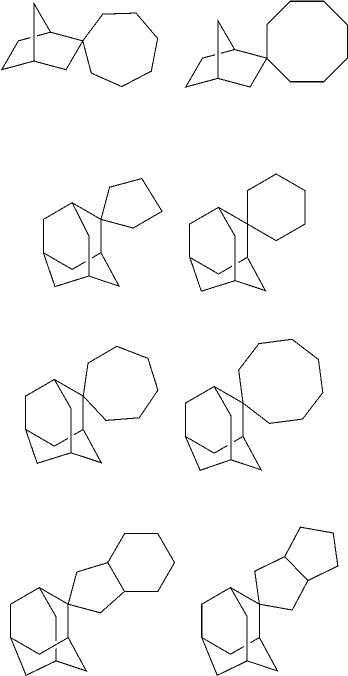

Examples of the alicyclic hydrocarbon group include a divalent to pentavalent group such as a cycloalkanediyl group, a cycloalkanetriyl group, a cycloalkanetetrayl group and a cycloalkanepentayl group.

Specific examples thereof include: a monocyclic alicyclic hydrocarbon group such as a cyclobutane-1,3-diyl group, a cyclopentane-1,3-diyl group, a cyclohexane-1,4-diyl group and a cyclooctane-1,5-diyl group; and
a polycyclic alicyclic hydrocarbon group such as a spiro ring having a cycloalkanediyl group bonded by spiro to each of a cycloalkanediyl group, a norbornanediyl group or an adamantanediyl group, such as a norbornane-1,4-diyl group, a norbornane-2,5-diyl group, an adamantane-1,5-diyl group, an adamantane-2,6-diyl group, a decahydronaphthalenediyl group, a bicyclo[3.3.0]octanediyl group, a spirocyclohexane-1,2′-cyclopentane-diyl group, or a spiroadamantane-2,3′-cyclopentane-diyl group.

In addition, examples thereof also include a group in which one or more hydrogen atoms of the above groups are replaced with bonding sites.

The number of carbon atoms of the alicyclic hydrocarbon group is preferably 3 to 20, is more preferably 3 to 18, is further preferably 3 to 16, and is further more preferably 3 to 12.

Examples of the combined group of two or more include a combined group of an alicyclic hydrocarbon group and a chain hydrocarbon group. In the combination, two or more types of alicyclic hydrocarbon groups and chain hydrocarbon groups may each be combined. In addition, any of the groups may be bonded to X.

Examples of the combined group of an alicyclic hydrocarbon group and a chain hydrocarbon group include a group in which an alicyclic hydrocarbon group is bonded to a chain hydrocarbon group (for example, *-chain hydrocarbon group-alicyclic hydrocarbon group- and the like), a group in which a chain hydrocarbon group is bonded to an alicyclic hydrocarbon group (for example, *-alicyclic hydrocarbon group-chain hydrocarbon group- and the like), a group in which an alicyclic hydrocarbon group and a chain hydrocarbon group are bonded to a chain hydrocarbon group (for example, *-chain hydrocarbon group-alicyclic hydrocarbon group-chain hydrocarbon group- and the like), a group in which a chain hydrocarbon group and an alicyclic hydrocarbon group are bonded to an alicyclic hydrocarbon group (for example, *-alicyclic hydrocarbon group-chain hydrocarbon group-alicyclic hydrocarbon group- and the like), a group in which an alicyclic hydrocarbon group, a chain hydrocarbon group and an alicyclic hydrocarbon group are bonded to a chain hydrocarbon group (for example, *-chain hydrocarbon group-alicyclic hydrocarbon group-chain hydrocarbon group-alicyclic hydrocarbon group- and the like), and a group in which a chain hydrocarbon group, an alicyclic hydrocarbon group and a chain hydrocarbon group are bonded to an alicyclic hydrocarbon group (for example, *-alicyclic hydrocarbon group-chain hydrocarbon group-alicyclic hydrocarbon group-chain hydrocarbon group- and the like).

* represents a bonding site to X⁴⁴.

In addition, examples thereof also include a group in which one or more hydrogen atoms of the above groups are replaced with bonding sites.

—CH₂— contained in the aliphatic hydrocarbon group having 1 to 28 carbon atoms of L⁴⁴ is optionally replaced with —O—, —S—, —SO—, —SO₂— or —CO—.

When —CH₂— contained in the aliphatic hydrocarbon group having 1 to 28 carbon atoms of L⁴⁴ is replaced with —O—, —S—, —SO—, —SO₂— or —CO—, the number of carbon atoms before the replacement corresponds to the defined number of carbon atoms of the aliphatic hydrocarbon group.

Examples of the group formed by replacing —CH₂— contained in the aliphatic hydrocarbon group with —O—, —S—, —SO₂—, —SO— or —CO— include a hydroxy group, a carboxy group, a thiol group, an alkoxy group, an alkylthio group, an alkoxycarbonyl group, an alkylsulfonyl group, an alkylcarbonyl group, an alkylcarbonyloxy group, an alkoxycarbonyloxy group, an oxy group, a carbonyl group, a thio group, a sulfonyl group, an alkanediyloxy group, an alkanediyloxycarbonyl group, an alkanediylcarbonyl group, an alkanediylcarbonyloxy group, an alkanediylsulfonyl group, an alkanediylthio group, a cycloalkoxy group, a cycloalkylalkoxy group, and a combined group of two or more of these groups. Examples of these groups formed by the replacement include the same groups as those exemplified in the present specification, within the range permitted by the upper limit of the number of carbon atoms.

In addition, examples thereof also include a group in which one or more hydrogen atoms of the above groups are replaced with bonding sites.

Examples of the group formed by replacing —CH₂— contained in the alicyclic hydrocarbon group with —O—, —S—, —SO—, —CO— or —SO₂— include the same groups as those exemplified in the present specification, within the range permitted by the upper limit of the number of carbon atoms.

The aliphatic hydrocarbon group in L⁴⁴ optionally has one or more substituents. Examples of the substituent include a halogen atom, a haloalkyl group having 1 to 4 carbon atoms, an alkyl group having 1 to 12 carbon atoms, an alicyclic hydrocarbon group having 3 to 18 carbon atoms, and an aromatic hydrocarbon group having 6 to 18 carbon atoms, where —CH₂— contained in the alkyl group or the alicyclic hydrocarbon group is optionally replaced with —O— or —CO—.

Examples of the halogen atom include a fluorine atom, a chlorine atom, a bromine atom and an iodine atom.

Examples of the haloalkyl group having 1 to 4 carbon atoms include a fluorinated alkyl group having 1 to 4 carbon atoms, a chlorinated alkyl group having 1 to 4 carbon atoms, a brominated alkyl group having 1 to 4 carbon atoms, and an iodinated alkyl group having 1 to 4 carbon atoms. Examples of the haloalkyl group include a perfluoroalkyl group having 1 to 4 carbon atoms (such as a trifluoromethyl group, a pentafluoroethyl group, a heptafluoropropyl group and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group and an iodomethyl group. The number of carbon atoms of the haloalkyl group is preferably 1 to 3, and is more preferably 1 or 2.

Examples of the alkyl group having 1 to 12 carbon atoms include a methyl group, an ethyl group, a propyl group, a butyl group, a pentyl group, a hexyl group, a heptyl group, an octyl group, a nonyl group, a decyl group, an undecyl group and a dodecyl group. The number of carbon atoms of the alkyl group is preferably 1 to 9, is more preferably 1 to 6, is further preferably 1 to 4, and is further more preferably 1 to 3.

Examples of the alicyclic hydrocarbon group having 3 to 18 carbon atoms include a monocyclic cycloalkyl group such as a cyclopropyl group, a cyclobutyl group, a cyclopentyl group, a cyclohexyl group, a cycloheptyl group, a cyclooctyl group and a cyclodecyl group, and a polycyclic cycloalkyl group such as a decahydronaphthyl group, an adamantly group and a norbornyl group. The number of carbon atoms of the alicyclic hydrocarbon group is preferably 3 to 16, is more preferably 3 to 12, and is further preferably 3 to 10.

Examples of the aromatic hydrocarbon group having 6 to 18 carbon atoms include a phenyl group, a naphthyl group, a biphenyl group, an anthryl group, a phenanthryl group and a binaphthyl group. The number of carbon atoms of the aromatic hydrocarbon group is preferably 6 to 14, is more preferably 6 to 12, and is further preferably 6 to 10.

When —CH₂— contained in the alkyl group is replaced with —O— or —CO—, the number of carbon atoms before the replacement is regarded as the total number of carbon atoms of the alkyl group. Examples of the group formed by the replacement include a hydroxy group, a carboxy group, an alkoxy group, an alkoxycarbonyl group, an alkylcarbonyl group, an alkylcarbonyloxy group and an alkoxycarbonyloxy group. Examples of these groups formed by the replacement include the same groups as those exemplified in the present specification, within the range permitted by the upper limit of the number of carbon atoms.

In addition, examples of the group formed by replacing —CH₂-contained in the alicyclic hydrocarbon group with —O— or —CO— include the same groups as those exemplified in the present specification, within the range permitted by the upper limit of the number of carbon atoms.

The substituent that the aliphatic hydrocarbon group in L⁴⁴ optionally has is preferably a halogen atom, a haloalkyl group having 1 to 3 carbon atoms, an alkyl group having 1 to 6 carbon atoms, an alicyclic hydrocarbon group having 3 to 10 carbon atoms or an aromatic hydrocarbon group having 6 to 10 carbon atoms, where —CH₂— contained in the alkyl group or the alicyclic hydrocarbon group is optionally replaced with —O— or —CO—, is more preferably a fluorine atom, an iodine atom, a perfluoroalkyl group having 1 to 3 carbon atoms or an alkyl group having 1 to 4 carbon atoms, where —CH₂— contained in the alkyl group is optionally replaced with —O— or —CO—, and is further preferably a fluorine atom, an iodine atom, a trifluoromethyl group, a methyl group, a hydroxy group or a methoxy group.

In a case where L⁴⁴ does not form an alicyclic hydrocarbon group together with the fluorinated alkyl group of R⁴⁶, it is preferably a chain hydrocarbon group having 1 to 12 carbon atoms and optionally having a substituent, where —CH₂— contained in the chain hydrocarbon group is optionally replaced with —O— or —CO—, an alicyclic hydrocarbon group having 3 to 18 carbon atoms and optionally having a substituent, where —CH₂— contained in the alicyclic hydrocarbon group is optionally replaced with —O— or —CO—, or a group formed by combining a chain hydrocarbon group having 1 to 6 carbon atoms and optionally having a substituent and an alicyclic hydrocarbon group having 3 to 18 carbon atoms and optionally having a substituent, where —CH₂— contained in the chain hydrocarbon group is optionally replaced with —O— or —CO—, and —CH₂— contained in the alicyclic hydrocarbon group is optionally replaced with —O— or —CO—, is more preferably a chain hydrocarbon group having 1 to 8 carbon atoms and optionally having a substituent, where —CH₂— contained in the chain hydrocarbon group is optionally replaced with —O— or —CO—, an alicyclic hydrocarbon group having 3 to 12 carbon atoms and optionally having a substituent, or a group formed by combining a chain hydrocarbon group having 1 to 6 carbon atoms and optionally having a substituent and an alicyclic hydrocarbon group having 3 to 18 carbon atoms and optionally having a substituent, where —CH₂— contained in the chain hydrocarbon group is optionally replaced with —O— or —CO—, and is further preferably a chain hydrocarbon group having 1 to 8 carbon atoms and optionally having a substituent, where —CH₂-contained in the chain hydrocarbon group is optionally replaced with —O— or —CO—, an alicyclic hydrocarbon group having 5 to 12 carbon atoms and optionally having a substituent, a group formed by combining a chain hydrocarbon group having 1 to 6 carbon atoms and an alicyclic hydrocarbon group having 5 to 12 carbon atoms and optionally having a substituent, where —CH₂— contained in the chain hydrocarbon group is optionally replaced with —O— or —CO—, or a group formed by combining an alicyclic hydrocarbon group having 5 to 12 carbon atoms and optionally having a substituent, a chain hydrocarbon group having 1 to 6 carbon atoms, and an alicyclic hydrocarbon group having 5 to 12 carbon atoms and optionally having a substituent, where —CH₂— contained in the chain hydrocarbon group is optionally replaced with —O— or —CO—.

Examples of the fluorinated alkyl group having 1 to 8 carbon atoms in R⁴⁵ include a difluoromethyl group, a trifluoromethyl group, a 1,1-difluoroethyl group, a 2,2-difluoroethyl group, a 2,2,2-trifluoroethyl group, a perfluoroethyl group, a 1,1,2,2-tetrafluoropropyl group, a 1,1,2,2,3,3-hexafluoropropyl group, a perfluoroethylmethyl group, a 1-(trifluoromethyl)-1,2,2,2-tetrafluoroethyl group, a perfluoropropyl group, a 1,1,2,2-tetrafluorobutyl group, a 1,1,2,2,3,3-hexafluorobutyl group, a 1,1,2,2,3,3,4,4-octafluorobutyl group, a perfluorobutyl group, a 1,1-bis(trifluoro)methyl-2,2,2-trifluoroethyl group, a 2-(perfluoropropyl)ethyl group, a 1,1,2,2,3,3,4,4-octafluoropentyl group, a perfluoropentyl group, a 1,1,2,2,3,3,4,4,5,5-decafluoropentyl group, a 1,1-bis(trifluoromethyl)-2,2,3,3,3-pentafluoropropyl group, a perfluoropentyl group, a 2-(perfluorobutyl)ethyl group, a 1,1,2,2,3,3,4,4,5,5-decafluorohexyl group, a 1,1,2,2,3,3,4,4,5,5,6,6-dodecafluorohexyl group, a perfluoropentylmethyl group, a perfluorohexyl group, a perfluoroheptyl group and a perfluorooctyl group. The number of carbon atoms of the fluorinated alkyl group is preferably 1 to 6, is more preferably 1 to 5, is further preferably 1 to 4, and is further more preferably 1 to 3.

Within the range permitted by the upper limit of the number of carbon atoms, examples of the fluorinated alkyl group having 1 to 6 carbon atoms in R⁴⁶ include the same fluorinated alkyl groups having 1 to 6 carbon atoms as those exemplified for the fluorinated alkyl group of R⁴⁵.

When R⁴⁶ is a fluorinated alkyl group having 1 to 6 carbon atoms, R⁴⁵ is preferably a fluorinated alkyl group having 1 to 6 carbon atoms, is more preferably a fluorinated alkyl group having 1 to 3 carbon atoms, is further preferably a trifluoromethyl group, a perfluoroethyl group or a perfluoropropyl group, and is further more preferably a trifluoromethyl group.

When R⁴⁶ is a hydrogen atom, R⁴⁵ is preferably a fluorinated alkyl group having 1 to 6 carbon atoms, is more preferably a fluorinated alkyl group having 1 to 5 carbon atoms, and is further preferably a fluorinated alkyl group having 3 to 5 carbon atoms.

When R⁴⁶ is bonded to L⁴⁴ to form an alicyclic hydrocarbon group having 3 to 12 carbon atoms, examples of the group formed when R⁴⁶ is bonded to L⁴⁴ include the groups represented by the following formulas. In the following formulas, * and ** represent bonding sites, one of the two ** represents a bonding site to a hydroxy group and the other represents a bonding site to R⁴⁵, and L^{44A} is a part of the hydrocarbon group of L⁴⁴ and represents a single bond or an alkanediyl group having 1 to 4 carbon atoms. The number of carbon atoms of the alicyclic hydrocarbon group formed when R⁴⁶ is bonded to L⁴⁴ is preferably 3 to 10, is more preferably 3 to 8, and is further preferably 3 to 6.

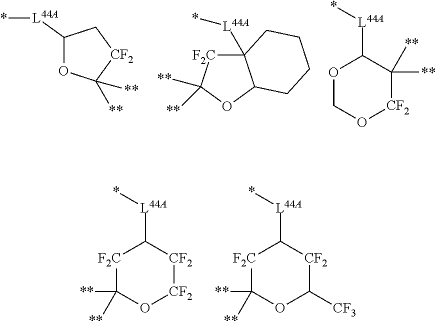

R⁴⁶ is preferably a hydrogen atom or a fluorinated alkyl group having 1 to 3 carbon atoms.

mi is preferably an integer of 1 to 3, is more preferably 1 or 2, and is further preferably 1.

Examples of the structural unit (a4-A) include the following structural units. Specific examples of the structural unit (a4-A) also include structural units in which the methyl group corresponding to R⁴⁴ in the following structural units is replaced with a hydrogen atom, a halogen atom, a haloalkyl group or an alkyl group other than a methyl group, and structural units in which the hydrogen atom corresponding to R⁴⁴ is replaced with a halogen atom, a haloalkyl group or an alkyl group.

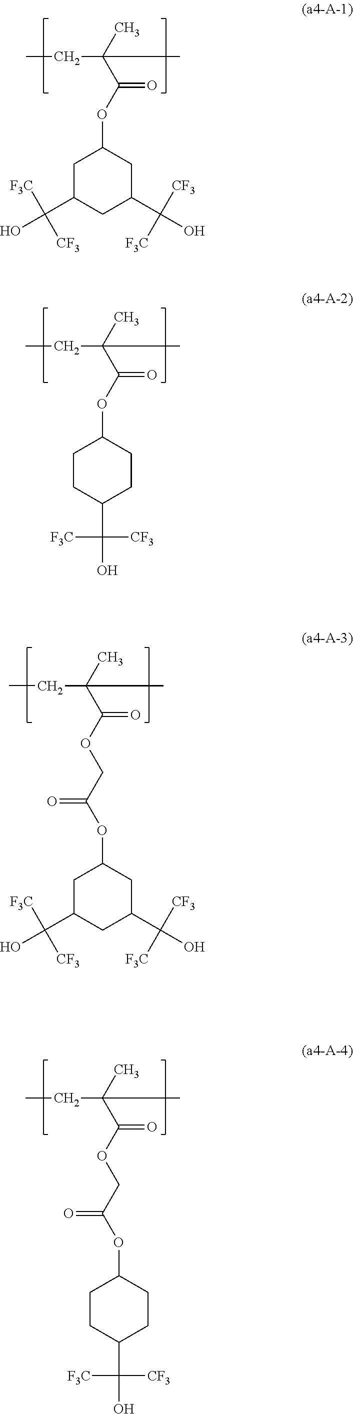

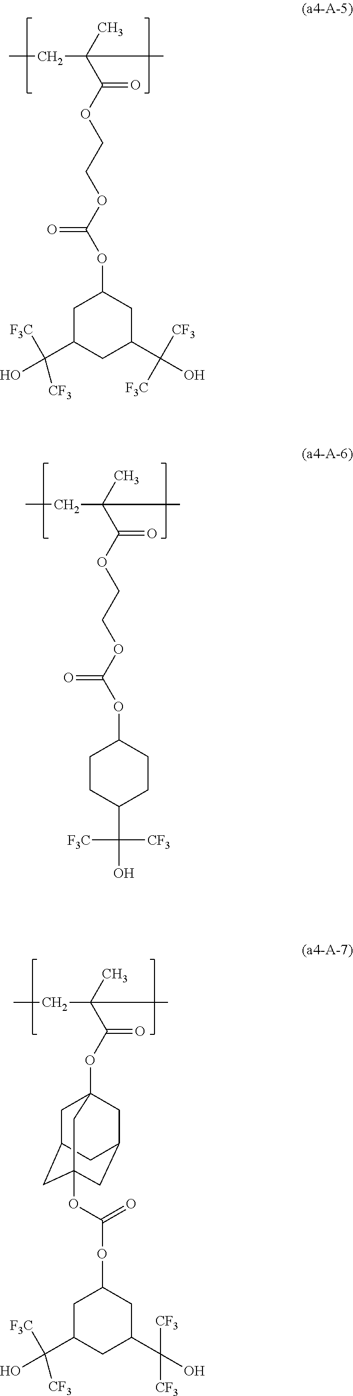

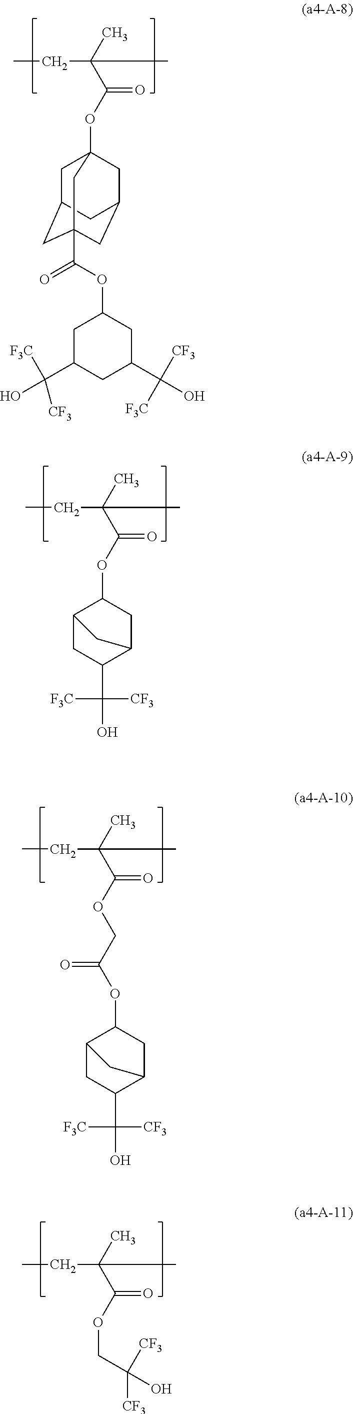

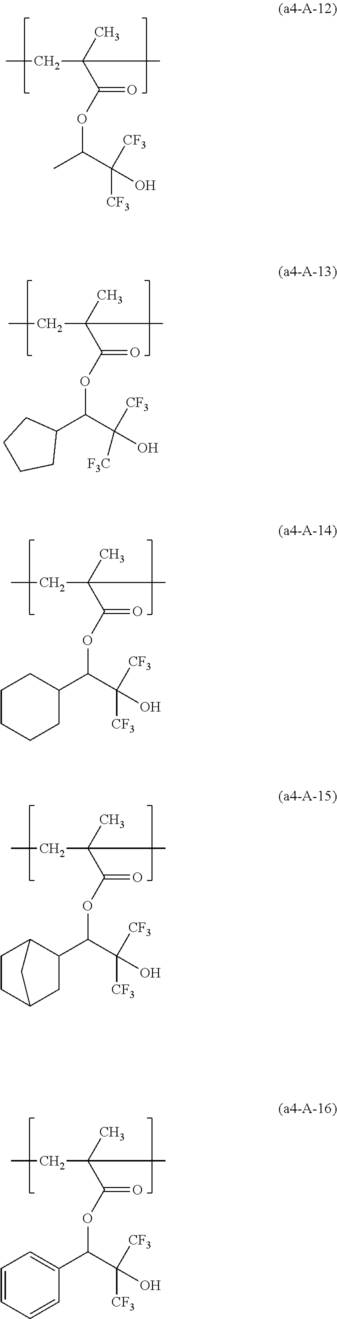

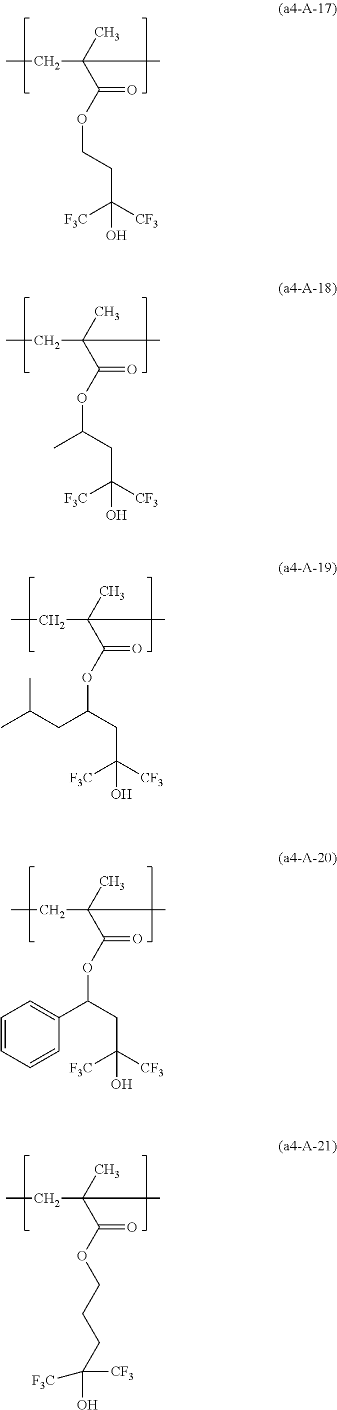

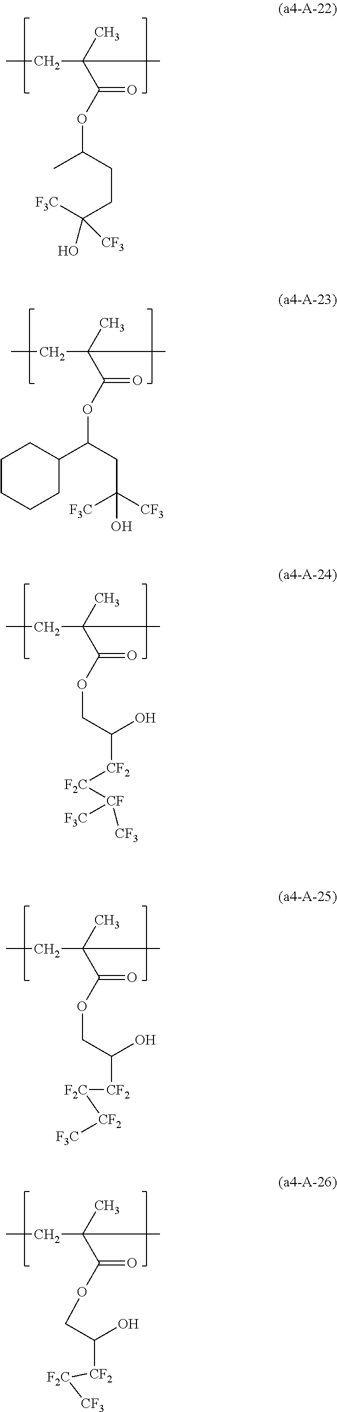

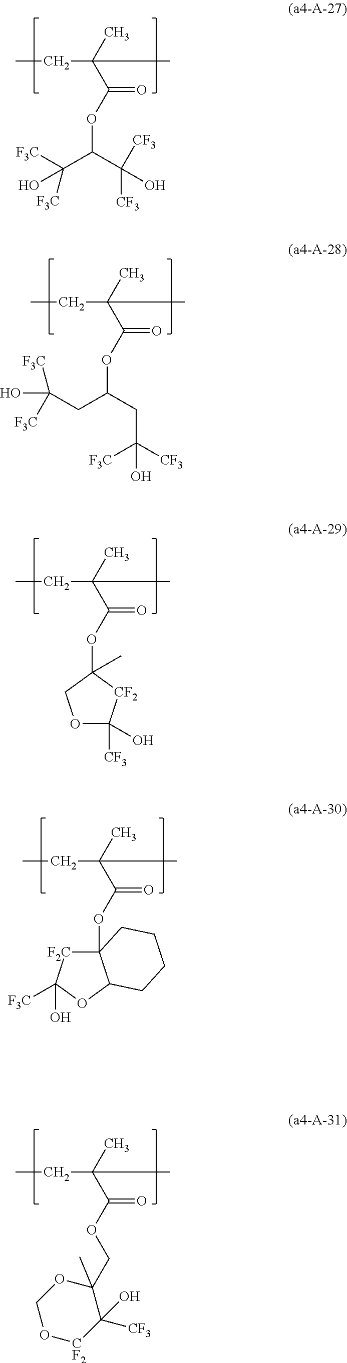

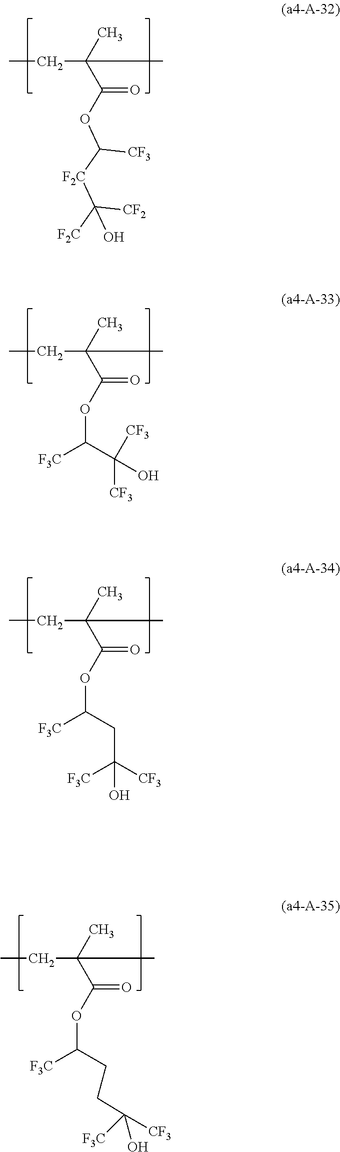

When the resin (A) and/or the like includes the structural unit (a4), the content ratio thereof, with respect to all structural units of the resin (A) and/or the like, is preferably 1 to 20 mol %, more preferably 2 to 15 mol %, and further preferably 3 to 10 mol %.

<Structural Unit (a5)>

Examples of the non-leaving hydrocarbon group that the structural unit (a5) has include a group having a straight-chain, branched or cyclic hydrocarbon group. In particular, the structural unit (a5) is preferably a group having an alicyclic hydrocarbon group.

Examples of the structural unit (a5) include a structural unit represented by formula (a5-1):

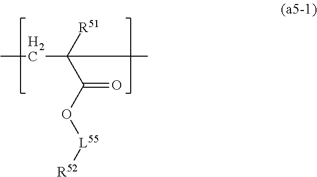

wherein
R⁵¹ represents a hydrogen atom or a methyl group;
R⁵² represents an alicyclic hydrocarbon group having 3 to 18 carbon atoms, and a hydrogen atom contained in the alicyclic hydrocarbon group is optionally replaced with an aliphatic hydrocarbon group having 1 to 8 carbon atoms; and
L⁵⁵ represents a single bond or a divalent saturated hydrocarbon group having 1 to 18 carbon atoms, and —CH₂— contained in the saturated hydrocarbon group is optionally replaced with —O— or —CO—.

The alicyclic hydrocarbon group in R⁵² may be either monocyclic or polycyclic. Examples of the monocyclic alicyclic hydrocarbon group include a cyclopropyl group, a cyclobutyl group, a cyclopentyl group and a cyclohexyl group. Examples of the polycyclic alicyclic hydrocarbon group include an adamantyl group and a norbornyl group.

Examples of the aliphatic hydrocarbon group having 1 to 8 carbon atoms include an alkyl group such as a methyl group, an ethyl group, a propyl group, an isopropyl group, a butyl group, a sec-butyl group, a tert-butyl group, a pentyl group, a hexyl group, an octyl group and a 2-ethylhexyl group.

Examples of the alicyclic hydrocarbon group having a substituent include a 3-methyladamantyl group.

R⁵² is preferably an unsubstituted alicyclic hydrocarbon group having 3 to 18 carbon atoms, and is more preferably an adamantyl group, a norbornyl group or a cyclohexyl group.

Examples of the divalent saturated hydrocarbon group in L⁵⁵ include a divalent chain saturated hydrocarbon group and a divalent alicyclic saturated hydrocarbon group, and it is preferably a divalent chain saturated hydrocarbon group.

Examples of the divalent chain saturated hydrocarbon group include an alkanediyl group such as a methylene group, an ethylene group, a propanediyl group, a butanediyl group and a pentanediyl group.

The divalent alicyclic saturated hydrocarbon group may be either monocyclic or polycyclic. Examples of the monocyclic alicyclic saturated hydrocarbon group include a cycloalkanediyl group such as a cyclopentanediyl group and a cyclohexanediyl group. Examples of the polycyclic divalent alicyclic saturated hydrocarbon group include an adamantanediyl group and a norbornanediyl group.

Examples of the group formed by replacing —CH₂— contained in the divalent saturated hydrocarbon group represented by L⁵⁵ with —O— or —CO— include the groups represented by formulas (L1-1) to (L1-4). In the following formulas, * and ** each represent a bonding site, and * represents a bonding site to an oxygen atom:

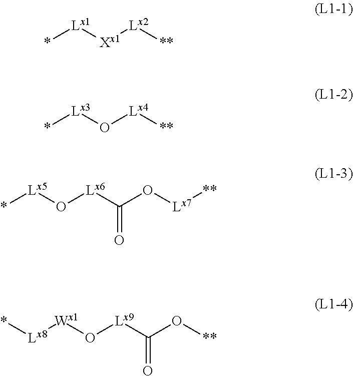

wherein, in formula (L1-1),
X^{x1} represents *—O—CO— or *—CO—O—, where * represents a bonding site to L^{x1};
L^{x1} represents a divalent aliphatic saturated hydrocarbon group having 1 to 16 carbon atoms; and L^{x2} represents a single bond or a divalent aliphatic saturated hydrocarbon group having 1 to 15 carbon atoms,
provided that the total number of carbon atoms of L^{x1} and L^{x2} is 16 or less,
wherein, in formula (L1-2),
L^{x3} represents a divalent aliphatic saturated hydrocarbon group having 1 to 17 carbon atoms; and
L^{x4} represents a single bond or a divalent aliphatic saturated hydrocarbon group having 1 to 16 carbon atoms,
provided that the total number of carbon atoms of L^{x3} and L^{x4} is 17 or less,
wherein, in formula (L1-3),
L^{x5} represents a divalent aliphatic saturated hydrocarbon group having 1 to 15 carbon atoms; and L^{x6} and L^{x1} each independently represent a single bond or a divalent aliphatic saturated hydrocarbon group having 1 to 14 carbon atoms,
provided that the total number of carbon atoms of L^{x5} L^{x6} and L^{x7} is 15 or less, and
wherein, in formula (L1-4),
L^{x8} and L^{x9} represent a single bond or a divalent aliphatic saturated hydrocarbon group having 1 to 12 carbon atoms; and
W^{x1} represents a divalent alicyclic saturated hydrocarbon group having 3 to 15 carbon atoms,
provided that the total number of carbon atoms of L^{x8}, L^{x9} and W^{x1} is 15 or less.

L^{x1} is preferably a divalent aliphatic saturated hydrocarbon group having 1 to 8 carbon atoms, and is more preferably a methylene group or an ethylene group.

L^{x2} is preferably a single bond or a divalent aliphatic saturated hydrocarbon group having 1 to 8 carbon atoms, and is more preferably a single bond.

L^{x3} is preferably a divalent aliphatic saturated hydrocarbon group having 1 to 8 carbon atoms.

L^{x4} is preferably a single bond or a divalent aliphatic saturated hydrocarbon group having 1 to 8 carbon atoms.

L^{x5} is preferably a divalent aliphatic saturated hydrocarbon group having 1 to 8 carbon atoms, and is more preferably a methylene group or an ethylene group.

L^{x6} is preferably a single bond or a divalent aliphatic saturated hydrocarbon group having 1 to 8 carbon atoms, and is more preferably a methylene group or an ethylene group.

L^{x7} is preferably a single bond or a divalent aliphatic saturated hydrocarbon group having 1 to 8 carbon atoms.

L^{x8} is preferably a single bond or a divalent aliphatic saturated hydrocarbon group having 1 to 8 carbon atoms, and is more preferably a single bond or a methylene group.

L^{x9} is preferably a single bond or a divalent aliphatic saturated hydrocarbon group having 1 to 8 carbon atoms, and is more preferably a single bond or a methylene group.

W^{x1} is preferably a divalent alicyclic saturated hydrocarbon group having 3 to 10 carbon atoms, and is more preferably a cyclohexanediyl group or an adamantanediyl group.

Examples of the group represented by formula (L1-1) include the divalent groups shown below.

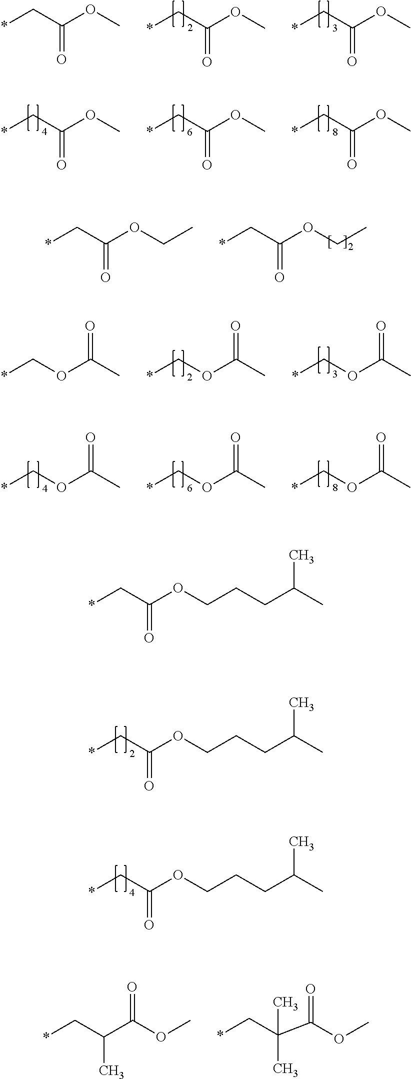

Examples of the group represented by formula (L1-2) include the divalent groups shown below.

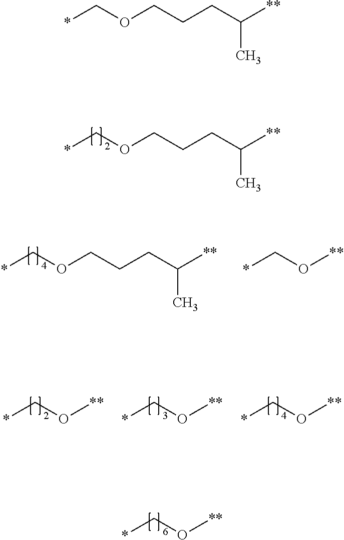

Examples of the group represented by formula (L1-3) include the divalent groups shown below.

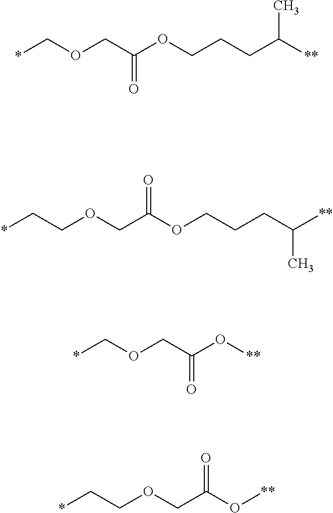

Examples of the group represented by formula (L1-4) include the divalent groups shown below.

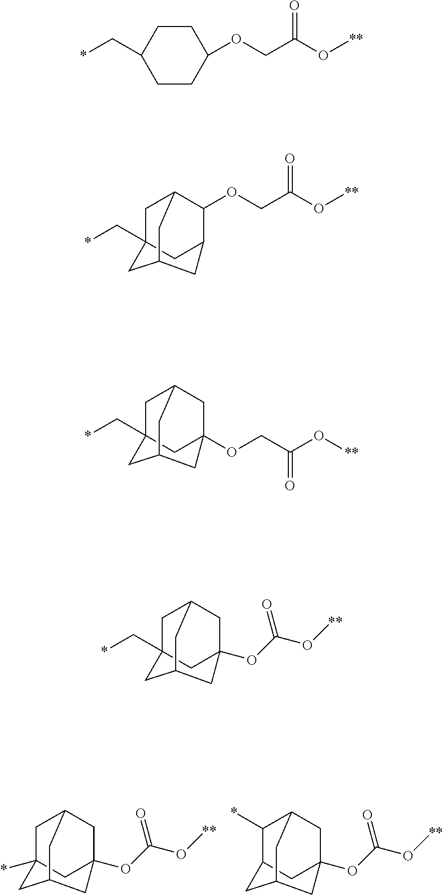

L⁵⁵ is preferably a single bond or a group represented by formula (L1-1).

Examples of the structural unit (a5-1) include the structural units shown below and the structural units in which the methyl group corresponding to R⁵¹ in the structural unit (a5-1) in the following structural units is replaced with a hydrogen atom.

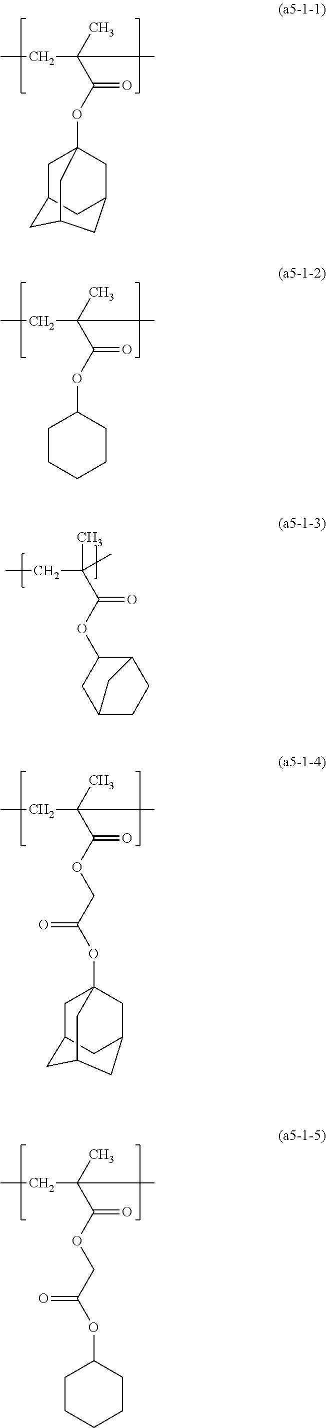

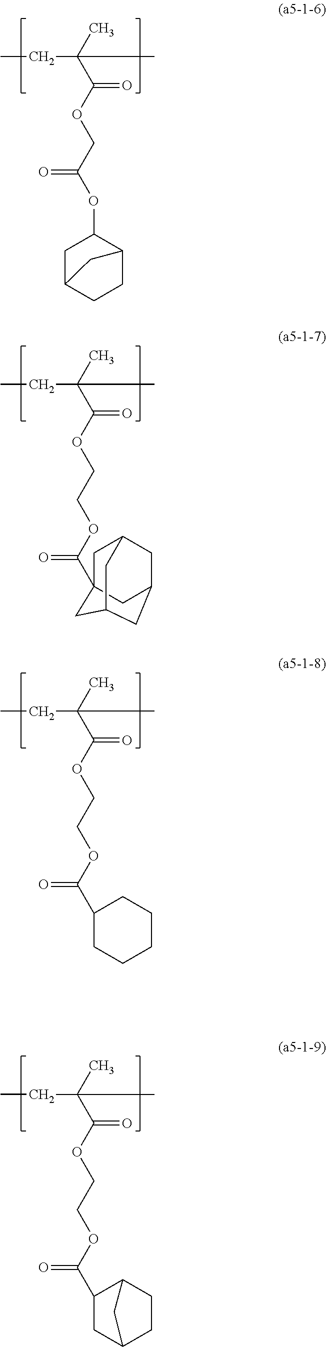

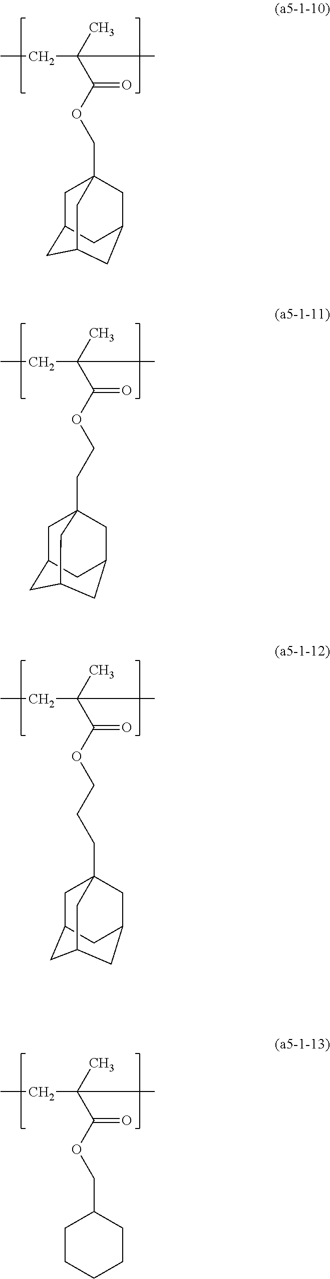

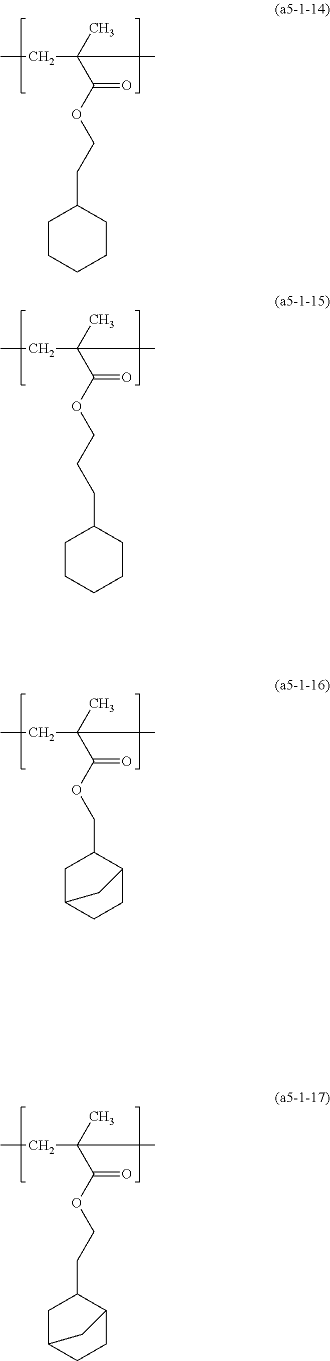

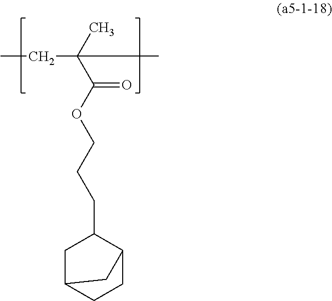

When the resin (A) and/or the like includes the structural unit (a5), the content ratio thereof, with respect to all structural units of the resin (A) and/or the like, is preferably 1 to 30 mol %, more preferably 2 to 20 mol %, and further preferably 3 to 15 mol %.

<Structural Unit (a6)>

The structural unit (a6) is a structural unit having a —SO₂— group, and preferably has a —SO₂— group in the side chain.

The structural unit having a —SO₂— group may have a straight-chain structure having a —SO₂— group, may have a branched structure having a —SO₂— group, or may have a cyclic structure (monocyclic or polycyclic structure) having a —SO₂— group. The above structure unit is preferably a structural unit having a cyclic structure having a —SO₂— group, and is more preferably a structural unit having a cyclic structure including —SO₂—O— (sultone ring).

Examples of the sultone ring include the rings represented by the following formulas (T¹-1), (T¹-2), (T¹-3) and (T¹-4). The bonding site can be set to any position. The sultone ring may be monocyclic, but is preferably polycyclic. The polycyclic sultone ring means a bridged ring including —SO₂—O— as an atomic group constituting the ring, and examples thereof include the rings represented by formulas (T¹-1) and (T¹-2). The sultone ring, like the ring represented by formula (T¹-2), may further include a heteroatom in addition to —SO₂—O— as an atomic group constituting the ring. Examples of the heteroatom include an oxygen atom, a sulfur atom and a nitrogen atom, and an oxygen atom is preferable.

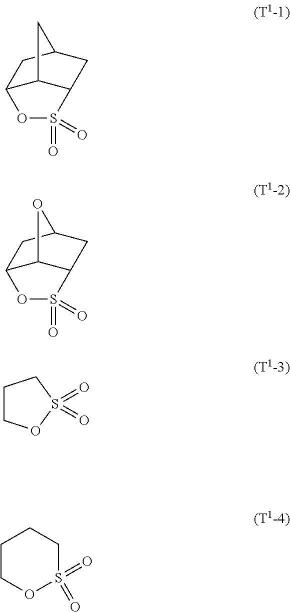 [SMILES: O=S1(=O)OC2CC3CC2C1C3 ; O=S1(=O)OC2CC3CC1C2O3 ; O=S1(=O)CCCO1 ; O=S1(=O)CCCCO1]

The sultone ring optionally has a substituent, and examples of the substituent include an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, and an alkylcarbonyl group having 2 to 4 carbon atoms. Examples of these groups include the same groups as those exemplified in the present specification, within the range permitted by the upper limit of the number of carbon atoms.

Examples of the aryl group include a phenyl group, a naphthyl group, an anthryl group, a p-methylphenyl group, a p-tert-butylphenyl group, a p-adamantylphenyl group, a tolyl group, a xylyl group, a cumyl group, a mesityl group, a biphenyl group, a phenanthryl group, a 2,6-diethylphenyl group and a 2-methyl-6-ethylphenyl group.

Examples of the aralkyl group include a benzyl group, a phenethyl group, a phenylpropyl group, a naphthylmethyl group and a naphthylethyl group.

From the viewpoint of ease of production of a monomer from which the structural unit (a6) is derived, a sultone ring having no substituent is preferable.

As the sultone ring, the ring represented by the following formula (T1′) is preferable:

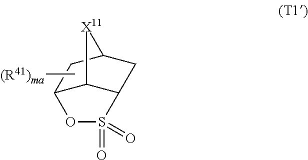

wherein
X¹¹ represents an oxygen atom, a sulfur atom or a methylene group;
R⁴¹ represents an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms;
ma represents an integer of 0 to 9, and when ma is 2 or larger, a plurality of R⁴¹ may be the same or different from each other; and
the bonding site is set to any position.

X¹¹ is preferably an oxygen atom or a methylene group, and is more preferably a methylene group.

Examples of R⁴¹ include the same substituents as those for the sultone ring, and it is preferably an alkyl group having 1 to 12 carbon atoms and optionally having a halogen atom or a hydroxy group.

ma is preferably 0 or 1, and is more preferably 0.

Examples of the ring represented by formula (T1′) include the following rings. The bonding site is set to any position. In particular, the bonding site is preferably the 1st or 3rd position.

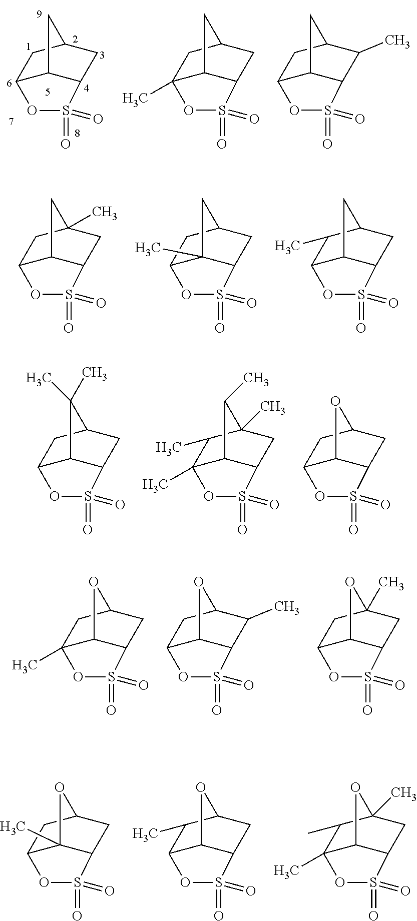

It is preferable that the structural unit having a —SO₂— group further has a group derived from a polymerizable group. Examples of the polymerizable group include a vinyl group, an acryloyl group, a methacryloyl group, an acryloyloxy group, a methacryloyloxy group, an acryloylamino group, a methacryloylamino group, an acryloylthio group and a methacryloylthio group.

Among these, the monomer from which the structural unit (a6) is derived is preferably a monomer having an ethylenically unsaturated bond, and is more preferably a (meth)acrylic monomer.

The structural unit (a6) is preferably a structural unit represented by formula (a6-0):

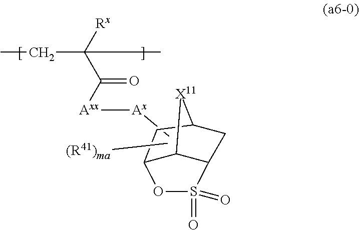

wherein
R^{x} represents an alkyl group having 1 to 6 carbon atoms and optionally having a halogen atom, a hydrogen atom or a halogen atom;
A^{xx} represents an oxygen atom, —N(R^{c})— or a sulfur atom;
A^{x} represents a single bond or a divalent saturated hydrocarbon group having 1 to 18 carbon atoms, and —CH₂— contained in the saturated hydrocarbon group is optionally replaced with —O—, —CO— or —N(R^{d})—;
X¹¹ represents an oxygen atom, a sulfur atom or a methylene group;
R⁴¹ has the same meaning as described above;
ma represents an integer of 0 to 9, and when ma is 2 or larger, a plurality of R⁴¹ may be the same or different from each other; and
R^{c} and R^{d} each independently represent a hydrogen atom or an alkyl group having 1 to 6 carbon atoms.

Examples of R^{x}, R^{c} and R^{d} include the same as those exemplified for R^{a01}, R^{a4} and R^{a5} in formulas (a1-0) to (a1-2).

R^{x} is preferably a hydrogen atom or an alkyl group having 1 to 4 carbon atoms, is more preferably a hydrogen atom, a methyl group or an ethyl group, and is further preferably a hydrogen atom or a methyl group.

Examples of the divalent saturated hydrocarbon group of A^{x} include a straight-chain alkanediyl group, a branched alkanediyl group, and a monocyclic or polycyclic divalent alicyclic saturated hydrocarbon group, and the above group may be a combination of two or more of these groups.

Specific examples thereof include: a straight-chain alkanediyl group such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, a heptadecane-1,17-diyl group, an ethane-1,1-diyl group, a propane-1,1-diyl group and a propane-2,2-diyl group;
a branched alkanediyl group such as a butane-1,3-diyl group, a 2-methylpropane-1,3-diyl group, a 2-methylpropane-1,2-diyl group, a pentane-1,4-diyl group and a 2-methylbutane-1,4-diyl group;
a monocyclic divalent alicyclic saturated hydrocarbon group that is a cycloalkanediyl group, such as a cyclobutane-1,3-diyl group, a cyclopentane-1,3-diyl group, a cyclohexane-1,4-diyl group, or a cyclooctane-1,5-diyl group; and
a polycyclic divalent alicyclic saturated hydrocarbon group such as a norbornane-1,4-diyl group, a norbornane-2,5-diyl group, an adamantane-1,5-diyl group and an adamantane-2,6-diyl group.

The bonding position of A^{x} to the sultone ring can be set to any position, but the 1st position is preferable.

Examples of the structural unit (a6-0) include the following structural units.

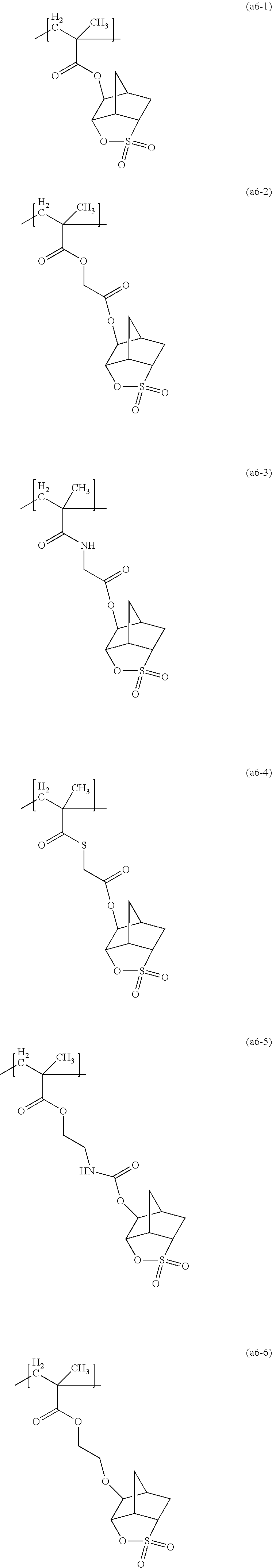

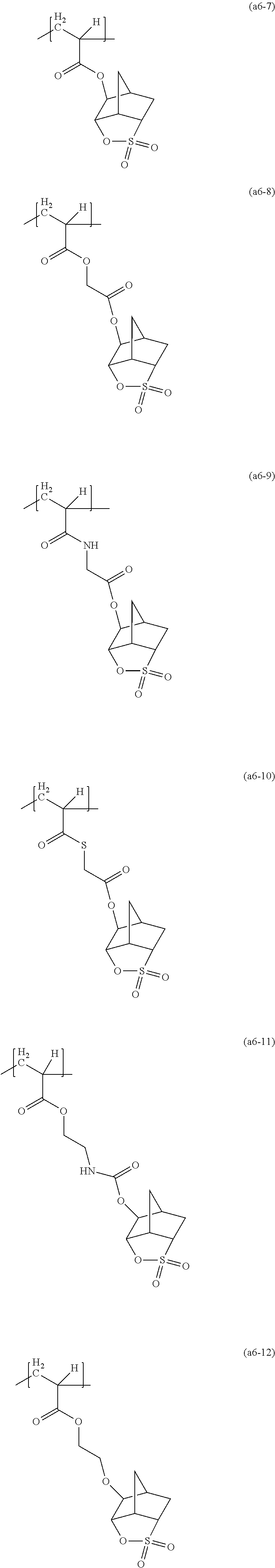

Among these, the structural units represented by formulas (a6-1), (a6-2), (a6-6), (a6-7), (a6-8) and (a6-12) are preferable, and the structural units represented by formulas (a6-1), (a6-2), (a6-7) and (a6-8) are more preferable.

When the resin (A) and/or the like includes the structural unit (a6), the content ratio thereof, with respect to all structural units of the resin (A) and/or the like, is preferably 1 to 50 mol %, more preferably 2 to 40 mol %, and further preferably 3 to 30 mol %.

<Structural Unit (a7)>

The resin (A) and/or the like may further contain a structural unit that decomposes upon exposure to generate acid (hereinafter referred to as “structural unit (a7)” in some cases). The structural unit (a7) functions in the resist composition in the same manner as an acid generating agent. Specific examples of the structural unit (a7) include the structural units disclosed in Japanese Patent Laid-Open No. 2016-79235, and it is preferable that the structural unit (a7) is a structural unit having a sulfonate anion or a carboxylate anion and an organic cation in a side chain, or a structural unit having a sulfonium cation and an organic anion in a side chain.

The structural unit having a sulfonate anion or a carboxylate anion and an organic cation in a side chain is preferably a structural unit represented by formula (a7-A):

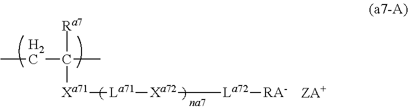

wherein
R^{a7} represents a hydrogen atom, a halogen atom, or an alkyl group having 1 to 6 carbon atoms and optionally having a halogen atom;
X^{a71} represents a single bond, —O—, —CO—, —S—, —SO—, —SO₂—, —NR^{a71}—, a phenylene group optionally having a substituent, or a combined group of these groups;
R^{a71} represents a hydrogen atom or an alkyl group having 1 to 6 carbon atoms;
L^{a71} and L^{a72} each independently represent a hydrocarbon group having 1 to 24 carbon atoms and optionally having a substituent, and —CH₂— contained in the hydrocarbon group is optionally replaced with —O—, —S—, —CO—, —SO— or —SO₂—;
X^{a72} represents * **—CO—O—, * **—O—CO—, * **—O—CO—O— or ***—O—, and *** represents a bonding site to L^{a71};
na7 represents an integer of 0 to 2, and when na7 is 2, a plurality of groups in parentheses may be the same or different from each other;
RA⁻ represents a sulfonate anion or a carboxyl anion; and
ZA⁺ represents an organic cation.

Examples of the halogen atom and the alkyl group optionally having a halogen atom in R^{a7} include the same groups as those exemplified in R^{a01} and the like in formula (a1-0).

R^{a7} is preferably a hydrogen atom or an alkyl group having 1 to 4 carbon atoms, is more preferably a hydrogen atom or an alkyl group having 1 to 3 carbon atoms, is further preferably a hydrogen atom, a methyl group or an ethyl group, and is further more preferably a hydrogen atom or a methyl group.

Examples of the alkyl group having 1 to 6 carbon atoms in R^{a71} include a methyl group, an ethyl group, a n-propyl group, an isopropyl group, a n-butyl group, a sec-butyl group, a tert-butyl group, a pentyl group and a hexyl group.

X^{a71} is preferably a single bond, —O—, —CO—, —NR^{a71}—, a phenylene group optionally having a substituent, or a combined group of these groups, and is more preferably a single bond, *—O—**, *—CO—O—**, *—O—CO—O—**, *—CO—NR^{a71}—**, *—NR^{a71}—CO—O—**, *—O—CO—NR^{a71}—** or *-Ax-Ph-Ay-**.

In the above groups, * and ** represent bonding sites, and * represents a bonding site to a carbon atom to which R^{a7} is bonded. Ax and Ay each independently represent one or more types of bonds selected from the group consisting of a single bond, an ether bond, a thioether bond, an amide bond, an ester bond and a carbonate ester bond. Ph represents a phenylene group optionally having a substituent.

When X^{a71} is a group represented by *-Ax-Ph-Ay-**, it is preferably a linking group represented by the following formula (X10):

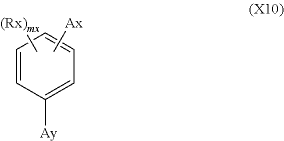

wherein
Ax and Ay have the same meanings as described above;
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms; and
mx represents an integer of 0 to 4, and when mx is an integer of 2 or larger, a plurality of Rx may be the same or different from each other.

When either Ax or Ay is a single bond, the other is preferably one or more selected from the group consisting of an ether bond, a thioether bond, an ester bond, a carbonate ester bond, and an amide bond.

When either Ax or Ay is an amide bond, a bond represented by —CO—NR^{a71}— is preferable.

The bonding position of Ay in the phenylene group is preferably the m-position or p-position with respect to the bonding position of Ax, and is more preferably the p-position.

In particular, Rx is preferably a fluorine atom, an iodine atom, a trifluoromethyl group, a methyl group or an ethyl group.

mx is preferably 0, 1 or 2.

Examples of X^{a71} include a single bond and the groups represented by the following formulas (X¹⁰-1) to (X¹⁰-10). * represents a bonding site to a carbon atom to which —R^{a7} is bonded. ** represents a bonding site to L^{a71}. X²⁰ represents —O— or —NR^{a71}—.

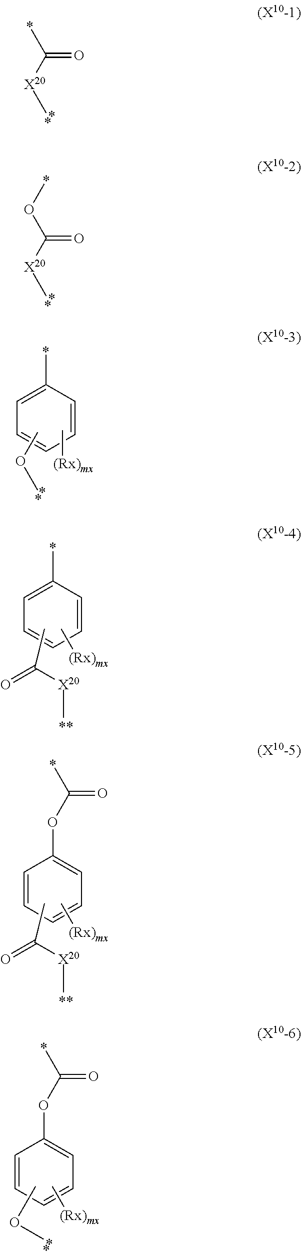

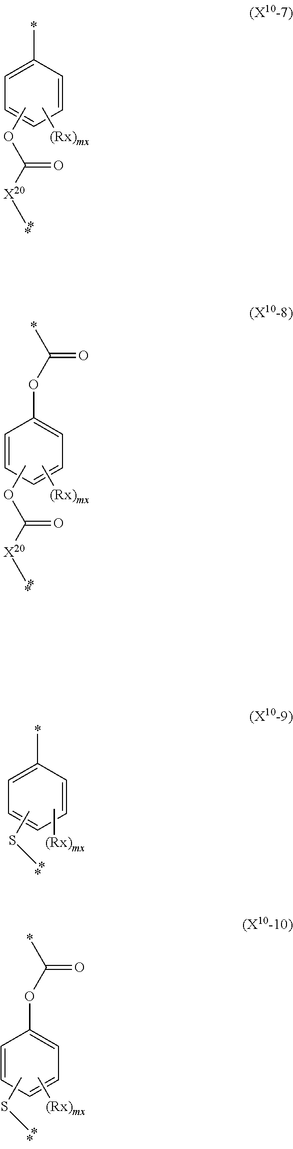

Specific examples of the groups represented by formulas (X¹⁰-1) to (X¹⁰-10) include the following groups.

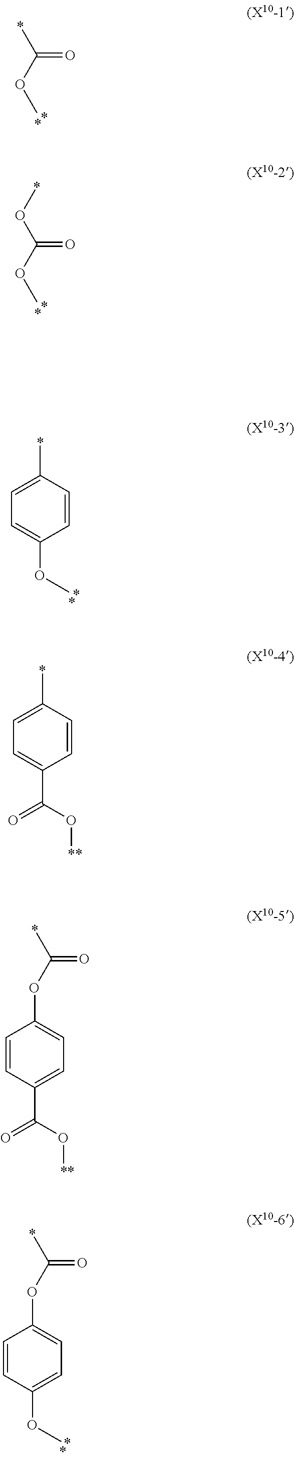

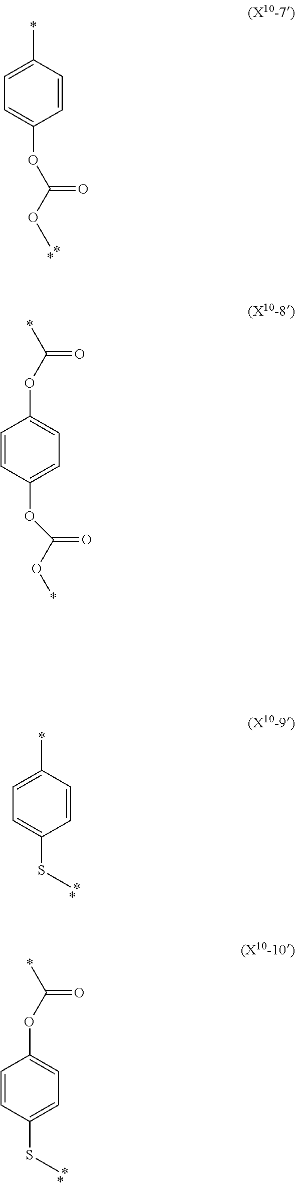

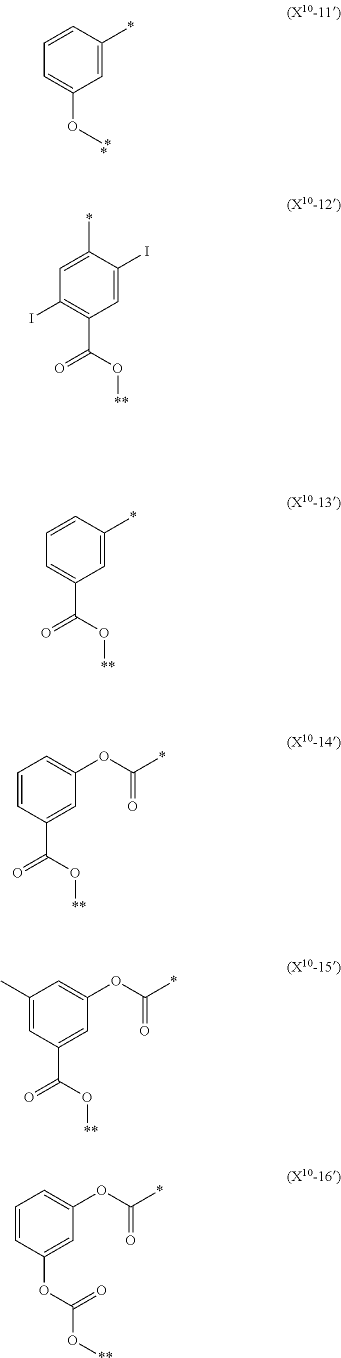

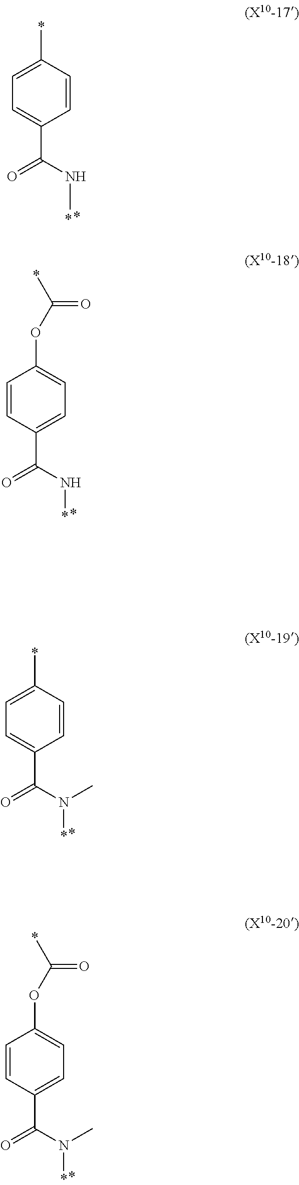 [SMILES: *c1ccc(C(=O)NC)cc1 ; *NC(=O)c1ccc(OC(*)=O)cc1 ; *c1ccc(C(=O)N(*)C)cc1 ; *C(=O)Oc1ccc(C(=O)N(*)C)cc1]

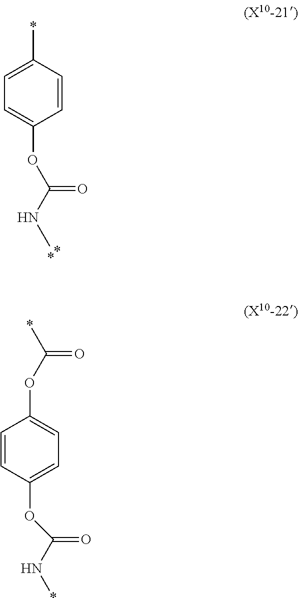 [SMILES: *c1ccc(OC(=O)NC)cc1 ; *NC(=O)Oc1ccc(OC(*)=O)cc1]

In particular, X^{a71} is preferably a single bond or a group represented by any one of formulas (X¹⁰-1′) and (X¹⁰-3′) to (X¹⁰-10′), is more preferably a single bond or a group represented by any one of formulas (X¹⁰-1′), (X¹⁰-4′), (X¹⁰-5′), (X¹⁰-6′) and (X¹⁰-10′), and is further preferably a single bond, a group represented by formula (X¹⁰-1′), a group represented by formula (X¹⁰-5′) or a group represented by formula (X¹⁰-6′).

Examples of the hydrocarbon group of L^{a71} include a group formed by removing one hydrogen atom from a chain hydrocarbon group, an alicyclic hydrocarbon group, an aromatic hydrocarbon group, or a combined group of two or more of these groups, the formed group being bonded to X^{a71} and X^{a72}. In addition, examples of the hydrocarbon group of L^{a72} include a group formed by removing one hydrogen atom from a chain hydrocarbon group, an alicyclic hydrocarbon group, an aromatic hydrocarbon group, or a combined group of two or more of these groups, the formed group being bonded to X^{a72} and RA⁻.

Examples of the chain hydrocarbon group of L^{a71} and L^{a72} include a group formed by removing one hydrogen atom from an alkyl group or an alkenyl group. The alkyl group may be a straight-chain or branched group, and specific examples thereof include a methyl group, an ethyl group, a n-propyl group, an iso-propyl group, a n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group and a heptadecyl group. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The number of carbon atoms of the chain hydrocarbon group is preferably 1 to 20, is more preferably 1 to 18, and is further preferably 1 to 10.

Examples of the alicyclic hydrocarbon group of L^{a71} and L^{a72} include a group formed by removing one hydrogen atom from a monocyclic or polycyclic cycloalkyl group. Examples of the monocyclic cycloalkyl group include a cyclobutyl group, a cycloheptyl group, a cyclohexyl group, a cyclopentyl group and a cyclooctyl group.

Examples of the polycyclic cycloalkyl group of L^{a71} and L^{a72} include a cycloalkyl group having a cross-linked structure, a cycloalkyl group in which two or more rings are condensed, and a cycloalkyl group in which two rings are bonded by Spiro. Examples of the cycloalkyl group having a cross-linked structure include a norbornyl group and an adamantyl group. Examples of the cycloalkyl group in which two or more rings are condensed include a bicyclo[4.4.0]decanyl group and a steroid group (steroid skeleton). In addition, examples of the cycloalkyl group in which two rings are bonded by spiro include a spirocyclic cycloalkyl group in which one type of cycloalkyl group selected from the group consisting of a cyclopentyl group, a cyclohexyl group, a norbornyl group and an adamantyl group, and a cycloalkyl group having 5 to 8 carbon atoms are bonded by spiro. Also, a double bond may be formed between two carbon atoms contained in the alicyclic hydrocarbon group. More specifically, examples thereof include the alicyclic hydrocarbon groups represented by the following formulas.

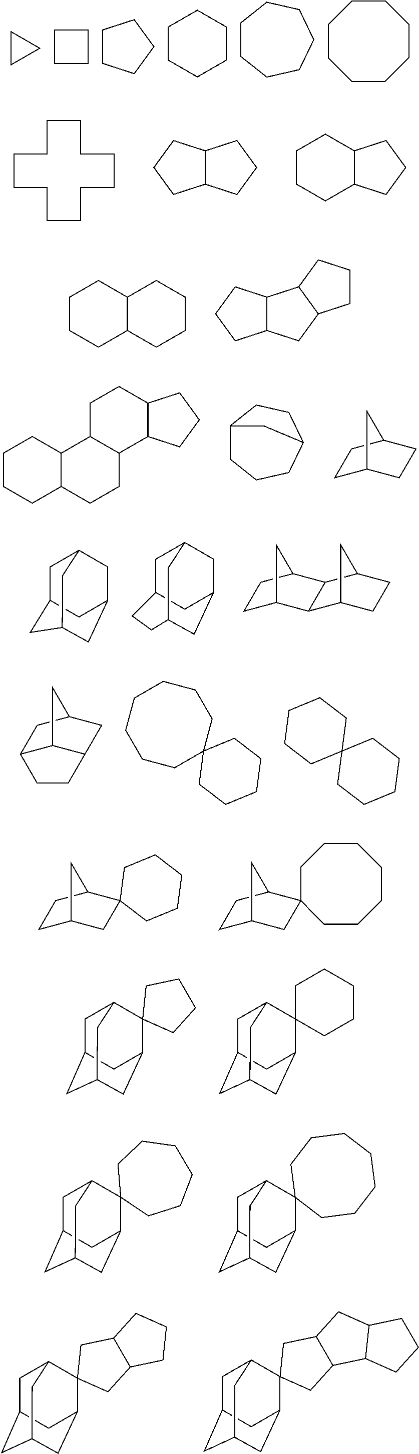

When the alicyclic hydrocarbon group is a monocyclic cycloalkyl group, the number of carbon atoms of the alicyclic hydrocarbon group is preferably 3 to 18, is more preferably 3 to 12, and is further preferably 3 to 8. When the alicyclic hydrocarbon group is a polycyclic cycloalkyl group, the number of carbon atoms of the alicyclic hydrocarbon group is preferably 6 to 18, and is more preferably 7 to 12.

Examples of the aromatic hydrocarbon group of L^{a71} and L^{a72} include a group formed by removing one hydrogen atom from an aryl group. Examples of the aryl group include a phenyl group, a naphthyl group, an anthryl group, a biphenyl group and a fluorenyl group. The number of carbon atoms of the aromatic hydrocarbon group is preferably 5 to 14, is more preferably 6 to 14, and is further preferably 6 to 10.

When —CH₂— contained in the hydrocarbon group of L^{a71} and L^{a72} is replaced with —O—, —CO—, —S—, —SO— or —SO₂—, the number of carbon atoms before replacement corresponds to the defined number of carbon atoms of the hydrocarbon group.

Among the hydrocarbon groups of L^{a71} and L^{a72}, examples of the group formed by replacing —CH₂— contained in the chain hydrocarbon group with —O—, —CO—, —S—, —SO— or —SO₂— include a hydroxy group, a carboxy group, a thiol group, a carbonyl group, an oxy group, a thio group, a sulfonyl group, an alkoxy group, an alkoxycarbonyl group, an alkylcarbonyl group, an alkylcarbonyloxy group, an alkanediyloxy group, an alkanediyloxycarbonyl group, an alkanediylcarbonyl group and an alkanediylcarbonyloxy group. Examples of these groups formed by the replacement include the same groups as those exemplified in the present specification, within the range permitted by the upper limit of the number of carbon atoms.

Among the hydrocarbon groups of L^{a71} and L^{a72}, examples of the group formed by replacing —CH₂— contained in the alicyclic hydrocarbon group, the aromatic hydrocarbon group and the combined group of these groups with —O—, —CO—, —S—, —SO— or —SO₂— include a group including a structure such as cyclic ether, cyclic ketone, cyclic ester (lactone), cyclic thioether, cyclic acetal or cyclic sulfonate ester (sultone), and a group derived from a furane ring, a thiophene ring, or the like. Examples of these groups formed by the replacement include the same groups as those exemplified in the present specification, within the range permitted by the upper limit of the number of carbon atoms.

Examples of the combined group of two or more groups selected from the group consisting of a chain hydrocarbon group, an alicyclic hydrocarbon group and an aromatic hydrocarbon group of L^{a71} and L^{a72} include a combined group of the above chain hydrocarbon group and the above alicyclic hydrocarbon group, a combined group of the above chain hydrocarbon group and the above aromatic hydrocarbon group, a combined group of the above alicyclic hydrocarbon group and the above aromatic hydrocarbon group, and a combined group of the above chain hydrocarbon group, the above alicyclic hydrocarbon group and the above aromatic hydrocarbon group. The combined group of an alicyclic hydrocarbon group and an aromatic hydrocarbon group may be a condensed ring.

Examples of the substituent that the hydrocarbon group of L^{a71} and L^{a72} optionally has include a halogen atom, a cyano group and a nitro group. Examples of the halogen atom include a fluorine atom, a chlorine atom, a bromine atom and an iodine atom.

When L^{a71} and L^{a72} are combined groups of an alicyclic hydrocarbon group or an aromatic hydrocarbon group and a chain hydrocarbon group, the chain hydrocarbon group may be regarded as a substituent possessed by the alicyclic hydrocarbon group or the aromatic hydrocarbon group. In addition, when —CH₂— in the chain hydrocarbon group included in the hydrocarbon group of L^{a71} and L^{a72} is replaced with —O—, —CO—, —S—, —SO— or —SO₂—, the hydrocarbon group of L^{a71} and L^{a72} can substantially have a substituent such as a hydroxy group, a carboxy group, an alkoxy group, an alkylcarbonyl group, an alkoxycarbonyl group, an alkylcarbonyloxy group, a thiol group, an alkylthio group or an alkylsulfonyl group.

na7 is preferably 0 or 1.

In formula (a7-A), examples of ZA⁺ include the same cations as those in the salt represented by formula (B1) and the like.

When the hydrocarbon group of L^{a71} and L^{a72} is a saturated hydrocarbon group in formula (a7-A), the same groups as those exemplified as the divalent linking group of A^{b7} in formula (a7-B) described below may be included.

Examples of the structural unit represented by formula (a7-A) include the structural unit represented by formula (a7-A1) and the like:

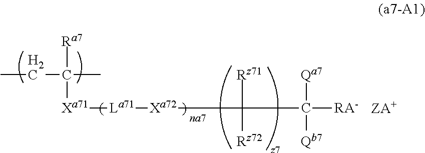

wherein
R^{a7}, X^{a71}, L^{a71}, X^{a72}, na7, RA⁻ and ZA⁺ have the same meanings as described above;
z7 represents an integer of 0 to 6; and
Q^{a7}, Q^{b7}, R^{z71} and R^{z72} each independently represent a hydrogen atom, a fluorine atom, a perfluoroalkyl group having 1 to 6 carbon atoms, or an alkyl group having 1 to 6 carbon atoms, and when z7 is 2 or larger, a plurality of R^{z71} and R^{z72} may be the same or different from each other.

Examples of the perfluoroalkyl group having 1 to 6 carbon atoms or the alkyl group having 1 to 6 carbon atoms in Q^{a7}, Q^{b7}, R^{z71} and R^{z72} include the same groups as those exemplified for R^{a7}.

Examples of the structural unit represented by formula (a7-A) include the following structural units, a structural unit in which the group corresponding to the methyl group of R^{a7} is replaced with a hydrogen atom, a halogen atom (for example, a fluorine atom) or an alkyl group having 1 to 6 carbon atoms and optionally having a halogen atom (for example, a trifluoromethyl group or the like), and the structural units disclosed in WO 2012/050015. ZA⁺ represents an organic cation.

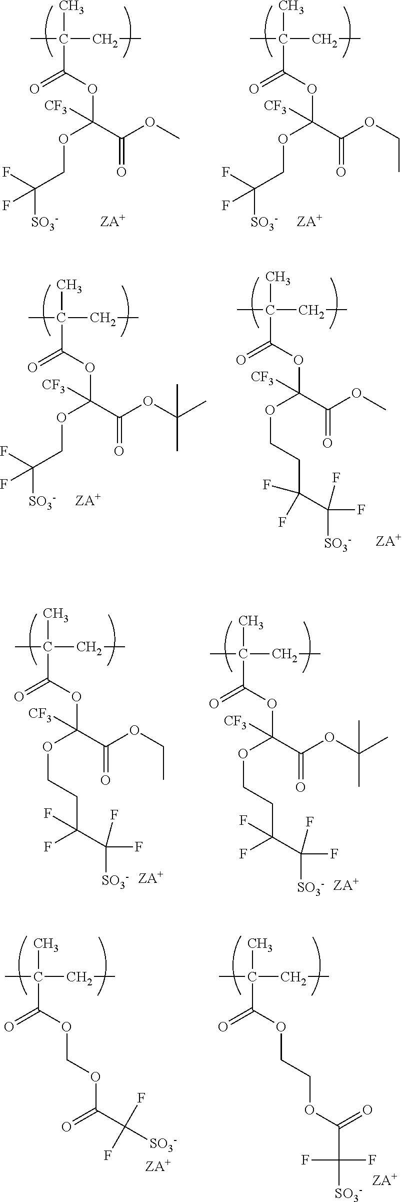

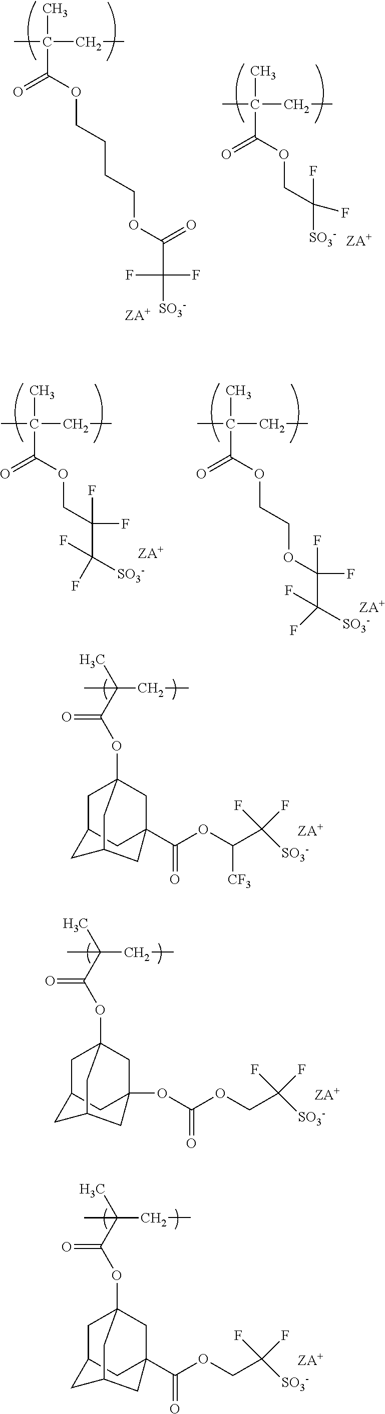

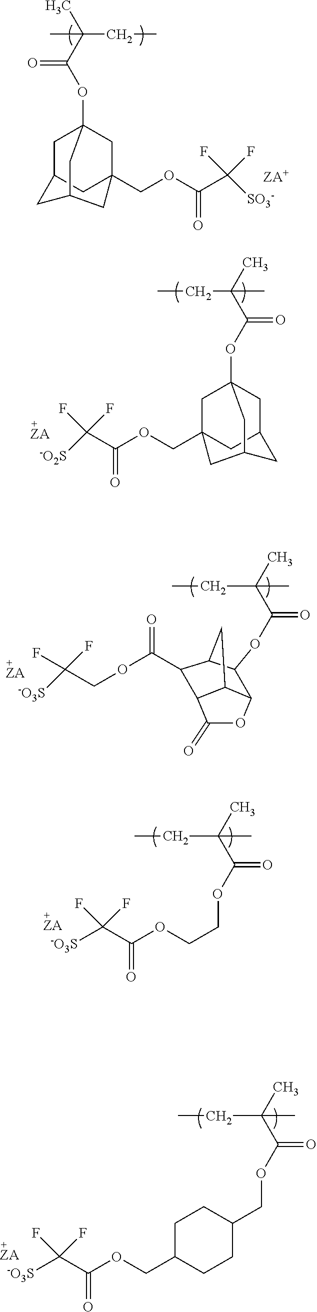

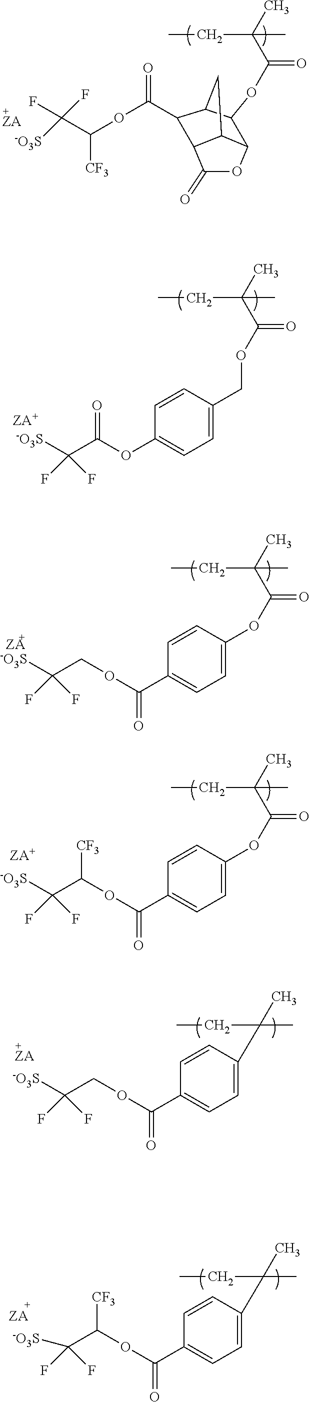

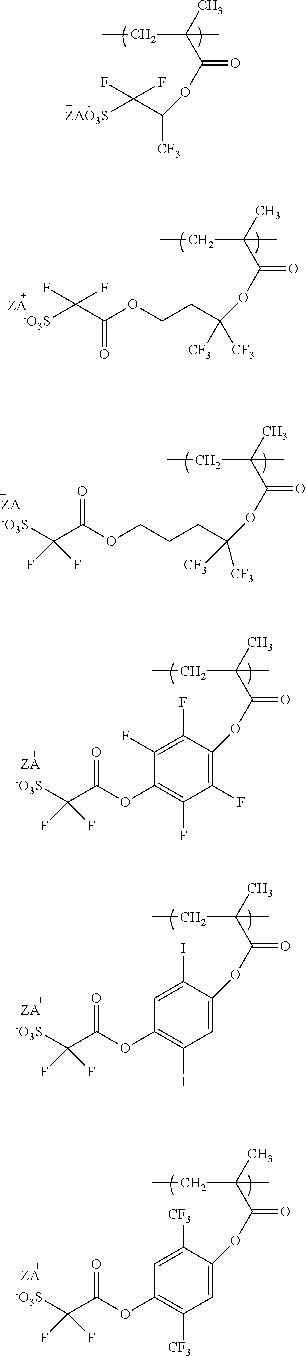

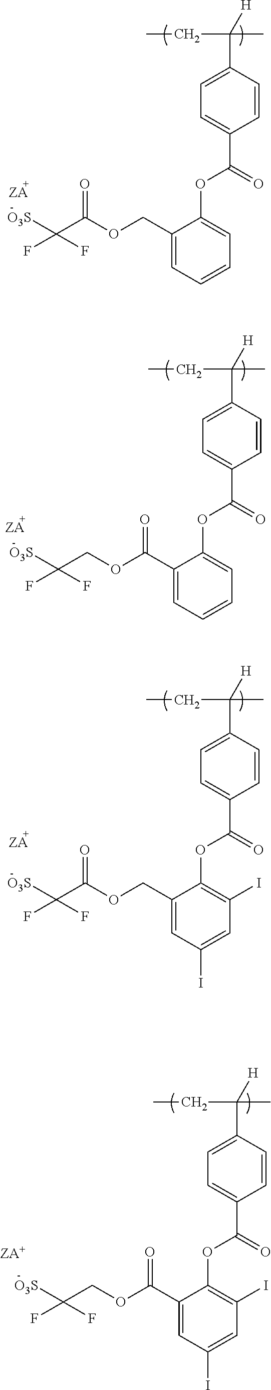

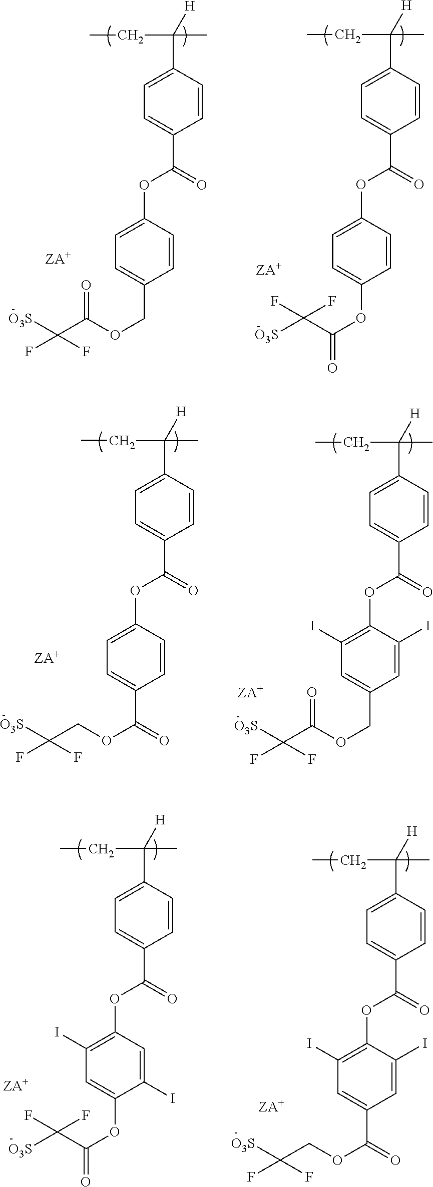

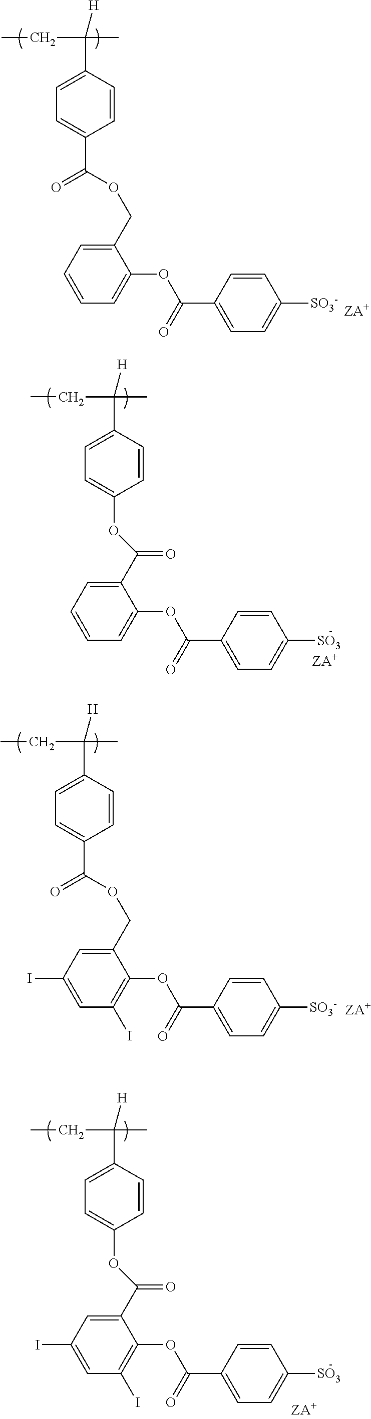

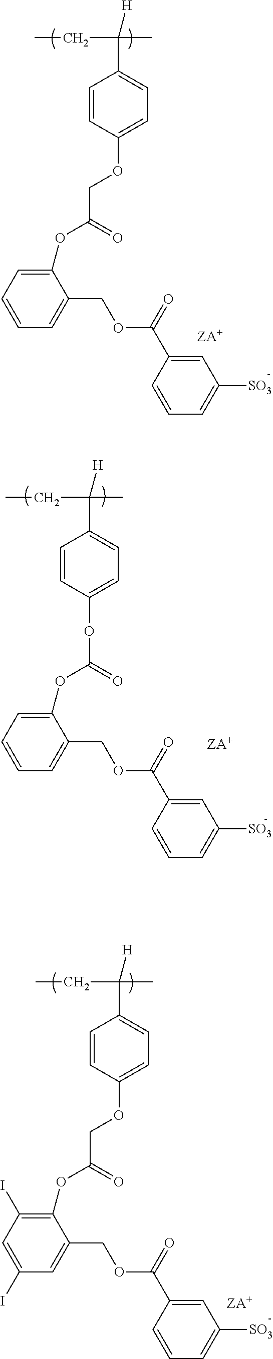

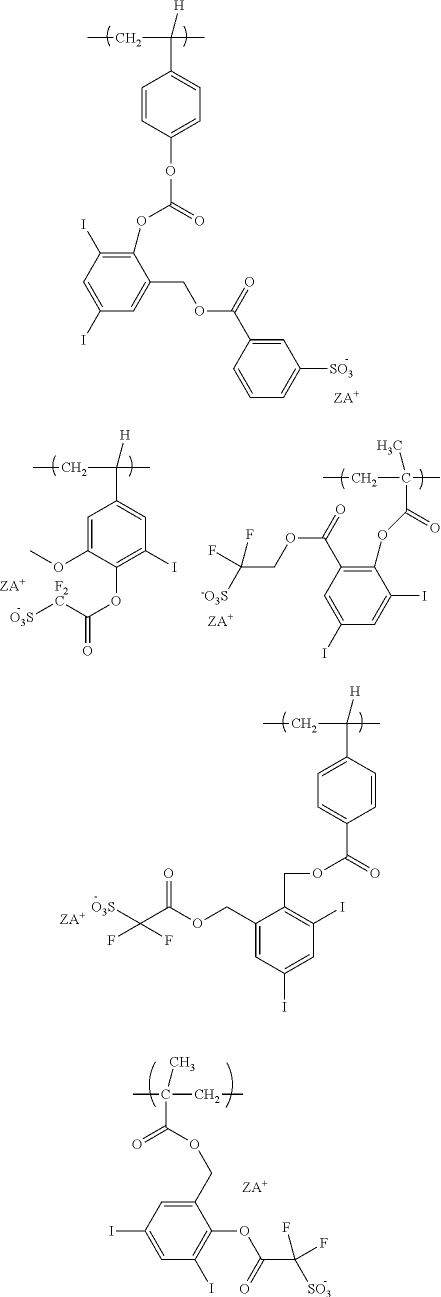

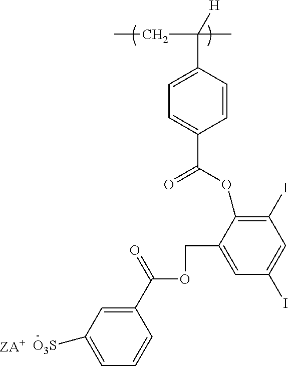

The structural unit having a sulfonio group and an organic anion in a side chain is preferably a structural unit represented by formula (a7-B):

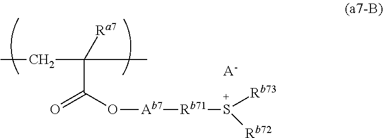

wherein
R^{a7} represents a hydrogen atom, a halogen atom, or an alkyl group having 1 to 6 carbon atoms and optionally having a halogen atom;
A^{b7} represents a single bond or a divalent linking group;
R^{b71} represents a divalent aromatic hydrocarbon group having 6 to 18 carbon atoms and optionally having a substituent;
R^{b72} and R^{b73} each independently represent a hydrocarbon group having 1 to 18 carbon atoms and optionally having a substituent, and R^{b72} and R^{b73} may be bonded to each other to form a ring together with a sulfur atom to which they are bonded; and
A⁻ represents an organic anion.

Examples of the halogen atom and the alkyl group optionally having a halogen atom of R^{a7} include the same halogen atoms and alkyl groups optionally having a halogen atom as those in formula (a7-A).

Examples of the divalent aromatic hydrocarbon group having 6 to 18 carbon atoms represented by R^{b71} include a phenylene group and a naphthylene group.

Examples of the hydrocarbon group represented by R^{b72} and R^{b73} include an alkyl group, an alicyclic hydrocarbon group, an aromatic hydrocarbon group, and a group formed by combining these groups.

Examples of the alkyl group, the alicyclic hydrocarbon group, the aromatic hydrocarbon group, and the group formed by combining these groups include the same as those described above.

Examples of the divalent linking group represented by A^{b7} include a divalent saturated hydrocarbon group having 1 to 18 carbon atoms, and —CH₂-contained in the divalent saturated hydrocarbon group is optionally replaced with —O—, —S—, —SO—, —SO₂— or —CO—.

Examples of the divalent saturated hydrocarbon group include a divalent chain saturated hydrocarbon group such as a straight-chain or branched alkanediyl group, and a monocyclic or polycyclic divalent alicyclic saturated hydrocarbon group, and it may be a combination thereof.

Specific examples thereof include: a straight-chain alkanediyl group such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; a branched alkanediyl group such as a butane-1,3-diyl group, a 2-methylpropane-1,3-diyl group, a 2-methylpropane-1,2-diyl group, a pentane-1,4-diyl group and a 2-methylbutane-1,4-diyl group; a divalent monocyclic alicyclic saturated hydrocarbon group that is a monocyclic cycloalkanediyl group such as a cyclobutane-1,3-diyl group, a cyclopentane-1,3-diyl group, a cyclohexane-1,4-diyl group and a cyclooctane-1,5-diyl group; and a divalent polycyclic alicyclic saturated hydrocarbon group that is a polycyclic cycloalkanediyl group such as a norbornane-1,4-diyl group, a norbornane-2,5-diyl group, an adamantane-1,5-diyl group and an adamantane-2,6-diyl group.

Examples of the group formed by replacing —CH₂— contained in the saturated hydrocarbon group with —O—, —S—, —SO—, —SO₂— or —CO— include the divalent groups shown below. Note that the number of carbon atoms before the replacement of —CH₂— contained in the saturated hydrocarbon group with —O—, —S—, —SO—, —SO₂— or —CO— is 17 or less. In the following formulas, * and ** represent bonding sites, and * represents a binding site to R^{b71}:

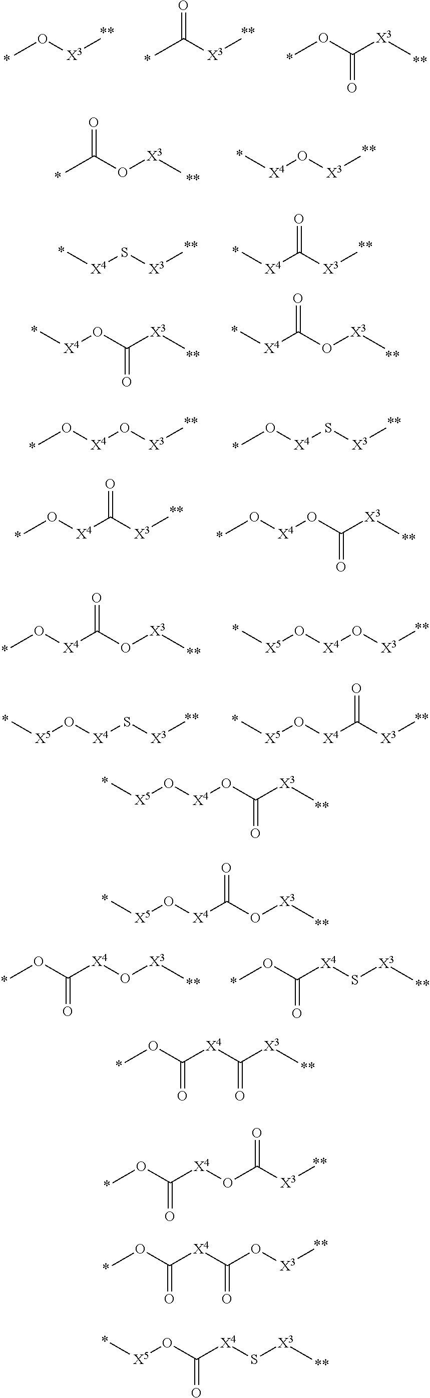

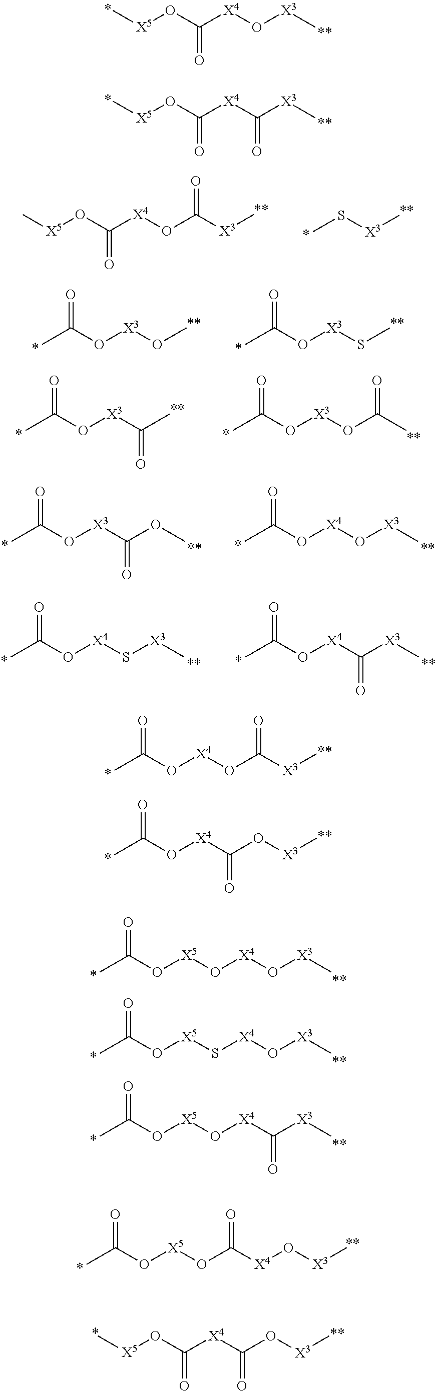

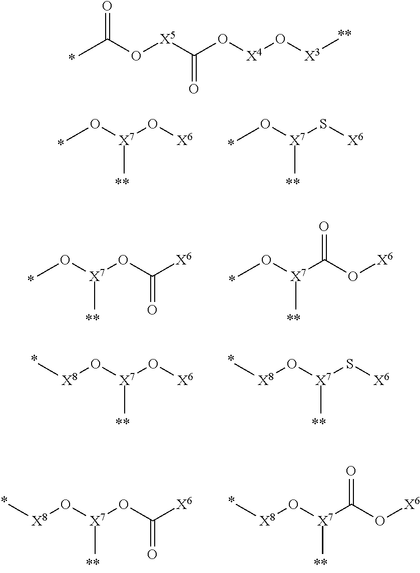

wherein X³ represents a divalent saturated hydrocarbon group having 1 to 16 carbon atoms;
X⁴ represents a divalent saturated hydrocarbon group having 1 to 15 carbon atoms;
X⁵ represents a divalent saturated hydrocarbon group having 1 to 13 carbon atoms;
X⁶ represents a divalent saturated hydrocarbon group having 1 to 14 carbon atoms;
X⁷ represents a trivalent saturated hydrocarbon group having 1 to 14 carbon atoms; and
X⁸ represents a divalent saturated hydrocarbon group having 1 to 13 carbon atoms.

Examples of the structural unit including a cation in formula (a7-B) include the structural units represented by the following formulas and structural units in which the group corresponding to the methyl group of R^{a7} is replaced with a hydrogen atom, a halogen atom (for example, a fluorine atom), or an alkyl group having 1 to 6 carbon atoms and optionally having a halogen atom (for example, a trifluoromethyl group or the like).

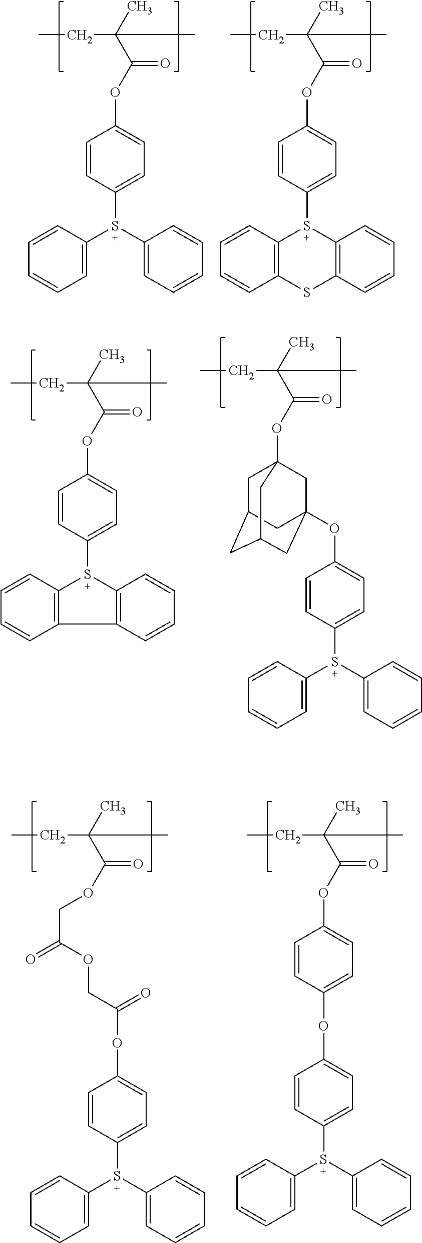

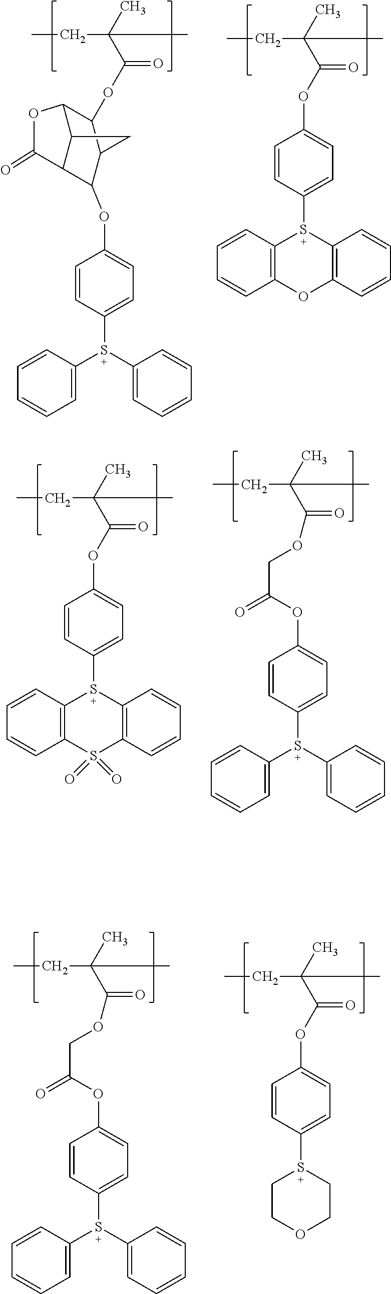

Examples of the organic anion represented by A⁻ include a sulfonate anion, a sulfonylimide anion, a sulfonylmethide anion, and a carboxylate anion. The organic anion represented by A⁻ is preferably a sulfonate anion, and the sulfonate anion is more preferably the same anion as those exemplified for the compound (B). Examples of the sulfonylimide anion, sulfonylmethide anion, and carboxylate anion include the same anions as those exemplified for the compound (B).

Examples of the structural unit represented by formula (a7-B) include the structural units represented below.

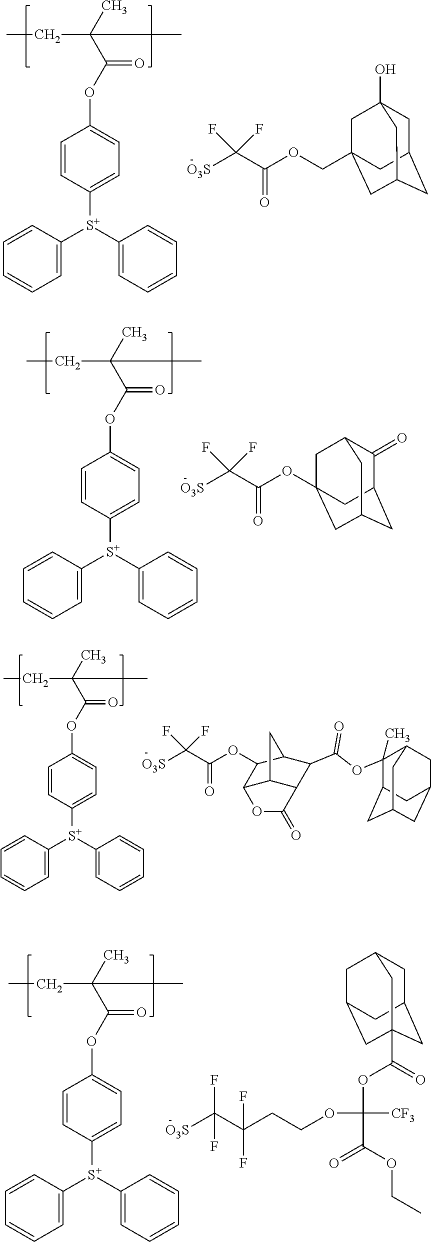

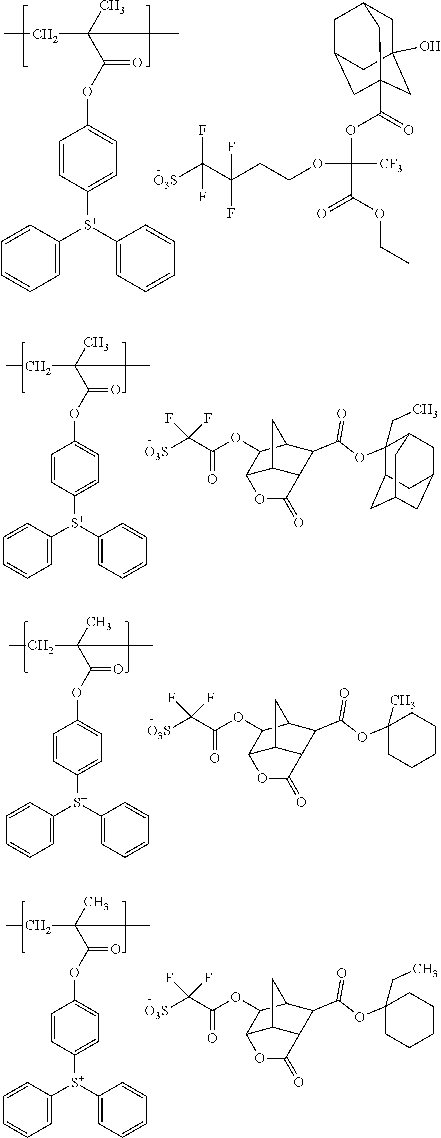

When the resin (A) and/or the like includes the structural unit (a7), the content ratio of the structural unit (a7), with respect to all structural units of the resin (A) and/or the like, is preferably 1 to 30 mol %, more preferably 2 to 25 mol %, and further preferably 3 to 20 mol %.

The resin (A) and/or the like is preferably a resin that includes the structural unit (a1). In particular, the resin (Ap) is more preferably a resin composed of the structural unit (IP), the structural unit (a1) and the structural unit (s), that is, a copolymer of the salt (I), the monomer (a1), and the monomer (s). The resin (A) that does not include the structural unit (IP) is preferably a resin composed of the structural unit (a1) and the structural unit (s), that is, a copolymer of the monomer (a1) and the monomer (s).

The structural unit (a1) is preferably at least one selected from the group consisting of the structural unit (a1-0), the structural unit (a1-1), the structural unit (a1-2) (preferably the structural unit having a cyclohexyl group or a cyclopentyl group), the structural unit represented by formula (a1-4), the structural unit represented by formula (a1-5), and the structural unit represented by formula (a1-6), and is more preferably at least one selected from the group consisting of the structural unit (a1-1) and the structural unit (a1-2).

The structural unit (s) is preferably at least one selected from the group consisting of the structural unit (a2) and the structural unit (a3). The structural unit (a2) is preferably the structural unit (a2-C) or the structural unit (a2-A). The structural unit (a3) is preferably at least one selected from the group consisting of the structural unit represented by formula (a3-1), the structural unit represented by formula (a3-2), and the structural unit represented by formula (a3-4).

Each structural unit constituting the resin (A) and/or the like may be used singly or in combination of two or more, and can be produced by a known polymerization method (for example, radical polymerization method) using monomers that lead to these structural units. The content ratio of each structural unit in the resin (A) and/or the like can be adjusted by the amount of monomer used in polymerization.

The weight average molecular weight of the resin (A) and the resin (Ap) is preferably 2,000 or more (more preferably 2,500 or more, further preferably 3,000 or more) and 50,000 or less (more preferably 30,000 or less, further preferably 15,000 or less), but oligomers with a lower weight average molecular weight may be included. In the present specification, the weight average molecular weight is a value determined by gel permeation chromatography under the conditions described in the Examples. The structural unit (IP) may constitute a dimer, a trimer, or a compound or oligomer with a weight average molecular weight of less than 2,000.

### <Acid Generating Agent>

The acid generating agent of the present invention is an acid generating agent containing the salt (I) or the structural unit (IP) of the present invention. The structural unit (IP) can be contained as a compound or resin in which a plurality of units are polymerized. In the acid generating agent, it may contain one type of salt (I) or two or more types of salt (I). One type of compound or resin containing the structural unit (IP) may be used alone, or two or more types may be used in combination. Furthermore, the acid generating agent of the present invention may contain both the salt (I) and the structural unit (IP).

The acid generating agent of the present invention may contain, in addition to the salt (I) and/or the structural unit (IP), a known compound that acts as an acid generating agent in the resist field (hereinafter referred to as “compound (B)” or “acid generating agent (B)” in some cases). One type of compound (B) may be used alone, or two or more types may be used in combination. The compound (B) will be described later.

When the acid generating agent contains the salt (I) and the compound (B), the ratio between the contents of the salt (I) and the compound (B) (mass ratio; salt (I):compound (B)) is usually 1:99 to 99:1, is preferably 2:98 to 98:2, is more preferably 5:95 to 95:5, is further preferably 10:90 to 90:10, is still more preferably 15:85 to 85:15, and is particularly preferably 40:60 to 60:40.

When the acid generating agent contains the structural unit (IP) and the compound (B), the ratio between the contents of the structural unit (IP) and the compound (B) (mass ratio; structural unit (IP):compound (B)) is usually 1:99 to 99:1, is preferably 2:98 to 98:2, is more preferably 5:95 to 95:5, is further preferably 10:90 to 90:10, is still more preferably 15:85 to 85:15, and is particularly preferably 40:60 to 60:40.

### [Resist Composition]

The resist composition of the present invention contains the acid generating agent of the present invention. The acid generating agent here may be the salt (I), or the resin (Ap) that includes the structural unit (IP). The resist composition of the present invention may contain a resin in addition to the acid generating agent of the present invention. The resin may be a resin that includes the structural unit (a1) having an acid labile group, or a resin that does not include the structural unit (a1). However, the resist composition of the present invention contains at least one of the salt (I) and the structural unit (IP), and may contain both. That is, the resist composition of the present invention may contain an acid generating agent containing the structural unit (IP) of the present invention or the salt (I) of the present invention. The structural unit (IP) may be in any form such as compound or resin. In other words, the resist composition of the present invention may contain, as an acid generating agent, the resin (Ap) and/or the resin (A), and the salt (I). It is preferable that the resist composition of the present invention contains a resin that includes the structural unit (a1) having an acid labile group. That is, it is preferable that the resin composition of the present invention contains at least
(a) the salt (I) and the resin (A) that includes the structural unit (a1) having an acid labile group,
(b) the resin (Ap) that includes the structural unit (IP) and the structural unit (a1) having an acid labile group, or
(c) the resin (Ap) that includes the structural unit (IP) and the resin (A) that includes the structural unit (a1) having an acid labile group.

In particular, the resin composition of the present invention is preferably a resist composition of (b). It may contain two or more types of resin (A) and/or resin (Ap).

It is preferable that the resist composition of the present invention further contains a quencher (hereinafter referred to as “quencher (C)” in some cases) and/or a solvent (hereinafter referred to as “solvent (E)” in some cases). In addition, the resist composition of the present invention may further contain a resin other than the resin (A) and/or the like described above.

<Resin Other than Resin (A) and/or the Like>

The resist composition of the present invention may contain a resin other than the resin (Ap) and the resin (A). Examples of the resin other than the resin (Ap) and the resin (A) include a resin that includes the same structural units as the resin (A) except that the structural unit (a1) is included in the resin (A) described above (hereinafter referred to as “resin (AX)” in some cases), and a resin that includes the structural unit (a4) and/or the structural unit (a5) (however, the structural unit (IP) and the structural unit (a1) are not included; hereinafter referred to as resin (X) in some cases).

Examples of the resin (AX) include a resin that includes the structural unit (a2), and a resin that includes the structural unit (a2-A) is preferable. In the resin (AX), the content ratio of the structural unit (a2-A) is preferably 5 mol % or more, more preferably 10 mol % or more, and further preferably 15 mol % or more, with respect to the total of all structural units in the resin (AX). In addition, the content ratio is preferably 80 mol % or less, and more preferably 70 mol % or less.

Examples of structural units that the resin (X) may further include the structural unit (a2), the structural unit (a3), and structural units derived from other known monomers. In particular, the resin (X) is preferably a resin including only the structural unit (a4) and/or the structural unit (a5), and is more preferably a resin including only the structural unit (a4).

When the resin (X) includes the structural unit (a4), the content ratio of the structural unit (a4) in the resin (X), with respect to the total of all structural units in the resin (X), is, for example, 20 mol % or more, preferably 30 mol % or more, more preferably 40 mol % or more, and further preferably 45 mol % or more. In addition, the content ratio is, for example, 100 mol % or less, preferably 80 mol % or less, more preferably 70 mol % or less, further preferably 60 mol % or less, and still more preferably 55 mol % or less. Specifically, the content ratio is, for example, 20 to 100 mol %, preferably 20 to 80 mol %, more preferably 30 to 70 mol %, further preferably 40 to 60 mol %, and still more preferably 45 to 55 mol %. When the resin (X) includes the structural unit (a5), the content ratio of the structural unit (a5), with respect to the total of all structural units in the resin (X), is, for example, 20 mol % or more, preferably 30 mol % or more, more preferably 40 mol % or more, and further preferably 45 mol % or more. In addition, the content ratio is, for example, 100 mol % or less, preferably 80 mol % or less, more preferably 70 mol % or less, further preferably 60 mol % or less, and still more preferably 55 mol % or less. Specifically, the content ratio is, for example, 20 to 100 mol %, preferably 20 to 80 mol %, more preferably 30 to 70 mol %, further preferably 40 to 60 mol %, and still more preferably 45 to 55 mol %. In addition, when the resin (X) includes the structural unit (a4) and the structural unit (a5), the total content ratio of the structural unit (a4) and the structural unit (a5), with respect to the total of all structural units in the resin (X), is, for example, 40 mol % or more, preferably 60 mol % or more, more preferably 70 mol % or more, and further preferably 80 mol % or more. Furthermore, the total content ratio is, for example, 100 mol % or less. Specifically, the total content ratio is, for example, 40 to 100 mol %, preferably 60 to 100 mol %, more preferably 70 to 100 mol %, and further preferably 80 to 100 mol %.

Each structural unit constituting the resin (AX) and the resin (X) may be used singly or in combination of two or more, and can be produced by a known polymerization method (for example, radical polymerization method) using monomers that derive these structural units. The content ratio of each structural unit in the resin (AX) and the resin (X) can be adjusted by the amount of monomer used in polymerization.

The weight average molecular weight of the resin (AX) and the resin (X) is preferably 6,000 or more (more preferably 7,000 or more) and 80,000 or less (more preferably 60,000 or less), but oligomers with a lower weight average molecular weight may be included. The method for measuring the weight average molecular weight of the resin (AX) and the resin (X) is the same as that for the resin (A) and/or the like.

When the resist composition of the present invention contains the resin (X), the content thereof is preferably 1 to 60 parts by mass, more preferably 1 to 50 parts by mass, further preferably 1 to 40 parts by mass, still more preferably 1 to 30 parts by mass, and still further preferably 1 to 8 parts by mass, with respect to 100 parts by mass of total of the resin (A) and/or the like.

The content ratio of the resin (A) and/or the like in the resist composition is preferably 60% by mass or more and 99% by mass or less, more preferably 70% by mass or more and 99% by mass or less, further preferably 80% by mass or more and 99% by mass or less, and further more preferably 90% by mass or more and 99% by mass or less, with respect to the solid content of the resist composition. The content ratio of the resin (Ap) is preferably 60% by mass or more and 99% by mass or less, more preferably 70% by mass or more and 99% by mass or less, further preferably 80% by mass or more and 99% by mass or less, and further more preferably 90% by mass or more and 99% by mass or less, with respect to the solid content of the resist composition. In addition, when a resin other than the resin (A) and/or the like is contained, the total content ratio of the resin (A) and/or the like and the resin other than the resin (A) and/or the like is preferably 60% by mass or more and 99% by mass or less, more preferably 70% by mass or more and 99% by mass or less, further preferably 80% by mass or more and 99% by mass or less, and further more preferably 90% by mass or more and 99% by mass or less, with respect to the solid content of the resist composition. The solid content of the resist composition and the resin content ratio therein can be measured by known analytical means such as liquid chromatography or gas chromatography.

### <Acid Generating Agent>

The acid generating agent of the present invention may be either an acid generating agent containing only the salt (I) and/or the structural unit (IP), or an acid generating agent containing the salt (I) and/or the structural unit (IP), and a known compound (B) that acts as an acid generating agent in the resist field.

The acid generating agent used in the resist composition of the present invention may be used alone, or in combination of two or more types.

### <Compound (B)>

As the compound (B), any one of a nonionic compound and an ionic compound may be used. Examples of the nonionic compound include a sulfonate ester (for example, 2-nitrobenzyl ester, aromatic sulfonate, oxime sulfonate, N-sulfonyloxyimide, sulfonyloxyketone and diazonaphthoquinone 4-sulfonate), and a sulfone (for example, disulfone, ketosulfone and sulfonyldiazomethane). Typical examples of the ionic compound include an onium salt containing an onium cation (for example, diazonium salt, phosphonium salt, sulfonium salt and iodonium salt). Examples of the anion of the onium salt include a sulfonate anion, a sulfonylimide anion, a sulfonylmethide anion and a carboxylate anion.

The compound (B) may be a part of the structural units constituting the resins contained in the resist composition, such as the structural unit (a7) described above.

As the compound (B), there can be used the compounds that generate an acid when exposed to radiation, such as those disclosed in Japanese Patent Laid-Open No. 63-26653, Japanese Patent Laid-Open No. 55-164824, Japanese Patent Laid-Open No. 62-69263, Japanese Patent Laid-Open No. 63-146038, Japanese Patent Laid-Open No. 63-163452, Japanese Patent Laid-Open No. 62-153853, Japanese Patent Laid-Open No. 63-146029, U.S. Pat. Nos. 3,779,778, 3,849,137, German Patent No. 3914407, and European Patent No. 126,712. In addition, the compound produced by a known method may be used.

The compound (B) may be an acid generating agent having an acid labile group. For example, the compound or resin serving as an acid generating agent may contain a partial structure serving as an acid labile group, and the acid labile group preferably contains the group (1) or the group (2), which is exemplified in the present specification. When the resist composition of the present invention contains a compound having an acid labile group, a resin that includes a structural unit having an acid labile group may be omitted.

Examples of one preferred form of the compound (B) include a salt represented by formula (B1) (hereinafter referred to as “salt (B1)”, “compound (B1)” or “acid generating agent (B1)” in some cases), a salt represented by formula (B2) (hereinafter referred to as “salt (B2)”, “compound (B2)” or “acid generating agent (B2)” in some cases), and a salt represented by formula (B4) (hereinafter referred to as “salt (B4)”, “compound (B4)” or “acid generating agent (B4)” in some cases):

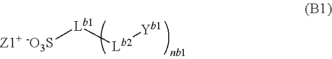

wherein
L^{b1} represents a single bond or a hydrocarbon group having a valence of (nb1+1) and optionally having a substituent, and —CH₂— contained in the hydrocarbon group is optionally replaced with —O—, —S—, —CO—, —SO— or —SO₂—;
L^{b2} represents a single bond or a divalent hydrocarbon group having 1 to 24 carbon atoms and optionally having a substituent, and —CH₂— contained in the hydrocarbon group is optionally replaced with —O—, —S—, —CO—, —SO— or —SO₂—;
Y^{b1} represents a methyl group optionally having a substituent, or a cyclic hydrocarbon group having 3 to 24 carbon atoms and optionally having a substituent, and —CH₂— contained in the cyclic hydrocarbon group is optionally replaced with —O—, —S—, —SO₂—, —SO— or —CO—;
nb1 represents an integer of 1 to 6, and when nb1 is 2 or larger, a plurality of groups in parentheses may be the same or different from each other; and
Z1+ represents an organic cation.

Examples of the hydrocarbon group having a valence of (nb1+1) of L^{b1} include a group formed by removing nb1 hydrogen atoms from a monovalent chain hydrocarbon group, a monovalent alicyclic hydrocarbon group, a monovalent aromatic hydrocarbon group, or a monovalent combined group of two or more of these groups, the formed group being bonded to one or more L^{b2}. The number of carbon atoms of the hydrocarbon group is preferably 1 to 48, is more preferably 1 to 42, is further preferably 1 to 36, is still more preferably 1 to 30, is still further preferably 1 to 24, and is further more preferably 1 to 18.

Examples of the chain hydrocarbon group include a group formed by removing nb1 hydrogen atoms from an alkyl group or an alkenyl group. The alkyl group may be a straight-chain or branched group, and specific examples thereof include a methyl group, an ethyl group, a n-propyl group, an iso-propyl group, a n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group and a heptadecyl group. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The number of carbon atoms of the chain hydrocarbon group is preferably 1 to 36, is more preferably 1 to 24, is further preferably 1 to 20, is still more preferably 1 to 18, is still further preferably 1 to 12, and is further more preferably 1 to 10.

Examples of the alicyclic hydrocarbon group include a group formed by removing nb1 hydrogen atoms from a monocyclic or polycyclic cycloalkyl group. Examples of the monocyclic cycloalkyl group include a cyclobutyl group, a cyclopentyl group, a cyclohexyl group, a cycloheptyl group and a cyclooctyl group.

Examples of the polycyclic cycloalkyl group include a cycloalkyl group having a cross-linked structure, a cycloalkyl group in which two or more rings are condensed, and a cycloalkyl group in which two rings are bonded by spiro. Examples of the cycloalkyl group having a cross-linked structure include a norbornyl group and an adamantyl group. Examples of the cycloalkyl group in which two or more rings are condensed include a bicyclo[4,4,0]decane group and a steroid group (steroid skeleton). In addition, examples of the cycloalkyl group in which two rings are bonded by spiro include a spirocyclic cycloalkyl group in which one type of cycloalkyl group selected from the group consisting of a cyclopentyl group, a cyclohexyl group, a norbornyl group and an adamantyl group, and a cycloalkyl group having 5 to 8 carbon atoms are bonded by spiro. Also, a double bond may be formed between two carbon atoms contained in the alicyclic hydrocarbon group.

More specifically, examples thereof include the alicyclic hydrocarbon groups represented by the following formulas.

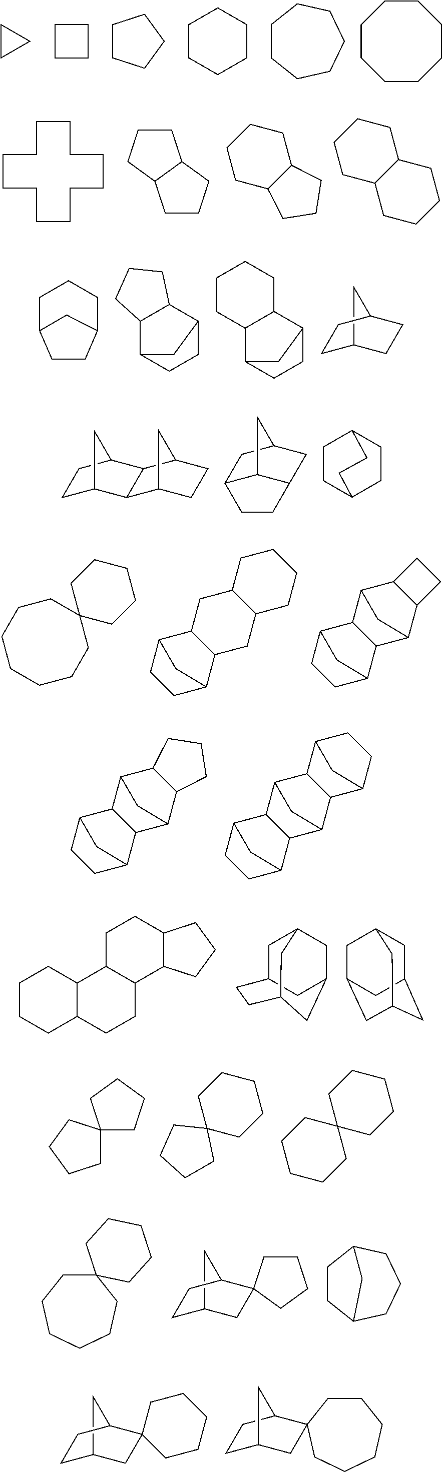

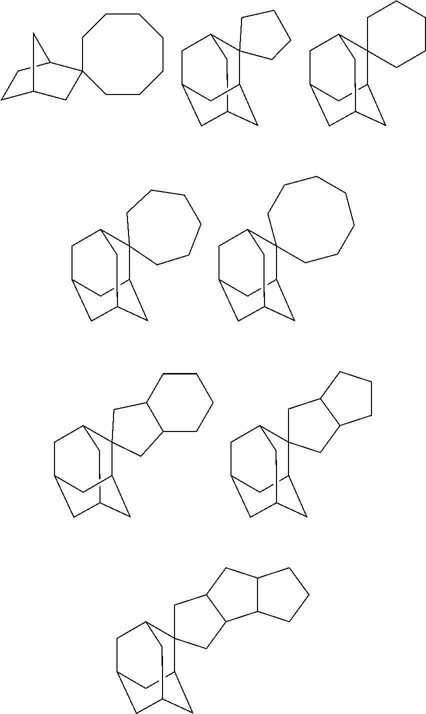

In a case where the alicyclic hydrocarbon group is a monocyclic cycloalkyl group, the number of carbon atoms of the alicyclic hydrocarbon group is preferably 3 to 24, is more preferably 3 to 20, is further preferably 3 to 18, is still more preferably 3 to 12, is still further preferably 3 to 10, and is further more preferably 3 to 8. In a case where the alicyclic hydrocarbon group is a polycyclic cycloalkyl group, the number of carbon atoms of the alicyclic hydrocarbon group is preferably 6 to 24, is more preferably 6 to 20, is further preferably 6 to 18, is still more preferably 6 to 12, and is still further preferably 7 to 12.

Examples of the aromatic hydrocarbon group include a group formed by removing nb1 hydrogen atoms from an aryl group. Examples of the aryl group include a phenyl group, a naphthyl group, an anthryl group, a biphenyl group and a fluorenyl group. More specifically, examples thereof include the aromatic hydrocarbon groups represented by the following formulas.

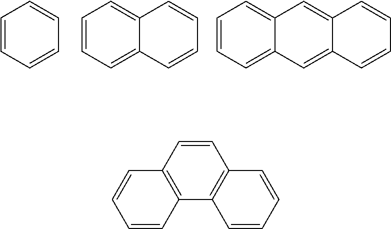

The number of carbon atoms of the aromatic hydrocarbon group is preferably 4 to 24, is more preferably 4 to 20, is further preferably 4 to 18, is still more preferably 5 to 14, is still further preferably 5 to 10, and is further more preferably 6 to 10.

When —CH₂— contained in the hydrocarbon group of L^{b1} is replaced with —O—, —CO—, —S—, —SO— or —SO₂—, the number of carbon atoms before replacement corresponds to the defined number of carbon atoms of the hydrocarbon group.

Among the hydrocarbon groups of L^{b1}, examples of the group formed by replacing —CH₂— contained in the chain hydrocarbon group with —O—, —CO—, —S—, —SO— or —SO₂— include a hydroxy group, a carboxy group, a carbonyl group, an oxy group, alkoxy group, an alkoxycarbonyl group, an alkylcarbonyl group, an alkylcarbonyloxy group, an alkoxycarbonyloxy group, an alkanediyloxy group, an alkanediyloxycarbonyl group, an alkanediylcarbonyl group and an alkanediylcarbonyloxy group. Examples of these groups formed by the replacement include the same groups as those exemplified in the present specification, within the range permitted by the upper limit of the number of carbon atoms.

Among the hydrocarbon groups of L^{b1}, examples of the group formed by replacing —CH₂— contained in the alicyclic hydrocarbon group, the aromatic hydrocarbon group or the combined group of these groups with —O—, —CO—, —S—, —SO— or —SO₂— include a group including a structure such as cyclic ether, cyclic ketone, cyclic ester (lactone), cyclic thioether, cyclic acetal or cyclic sulfonate ester (sultone), and a group derived from a furane ring, a thiophene ring, or the like. Examples of these groups formed by the replacement include the same groups as those exemplified in the present specification, within the range permitted by the upper limit of the number of carbon atoms.

Examples of the combined group of two or more groups selected from the group consisting of a chain hydrocarbon group, an alicyclic hydrocarbon group and an aromatic hydrocarbon group include a combined group of the above chain hydrocarbon group and the above alicyclic hydrocarbon group, a combined group of the above chain hydrocarbon group and the above aromatic hydrocarbon group, a combined group of the above alicyclic hydrocarbon group and the above aromatic hydrocarbon group, and a combined group of the above chain hydrocarbon group, the above alicyclic hydrocarbon group and the above aromatic hydrocarbon group. The combined group of an alicyclic hydrocarbon group and an aromatic hydrocarbon group may be a condensed ring.

Examples of the divalent hydrocarbon group of L^{b2} include a divalent chain hydrocarbon group, a divalent alicyclic hydrocarbon group, a divalent aromatic hydrocarbon group, and a combined group of two or more of these groups, and also include a group formed by removing one hydrogen atom from a monovalent hydrocarbon group, the formed group being bonded to Y^{b1}.

Examples of the divalent chain hydrocarbon group, the divalent alicyclic hydrocarbon group, the divalent aromatic hydrocarbon group and the divalent combined group of two or more of these groups of L^{b2} include a group formed by removing one hydrogen atom from the monovalent chain hydrocarbon group, the monovalent alicyclic hydrocarbon group, the monovalent aromatic hydrocarbon group and the monovalent combined group of two or more of these groups, respectively, which have been exemplified for L^{b1}, within the range permitted by the upper limit of the number of carbon atoms.

Examples of the substituent that the hydrocarbon group of L^{b1} and L^{b2} optionally has include a halogen atom, a cyano group and a nitro group. Examples of the halogen atom include a fluorine atom, a chlorine atom, a bromine atom and an iodine atom.

When L^{b1} and L^{b2} are combined groups of an alicyclic hydrocarbon group or an aromatic hydrocarbon group and a chain hydrocarbon group, the chain hydrocarbon group may be regarded as a substituent possessed by the alicyclic hydrocarbon group or the aromatic hydrocarbon group. In addition, when —CH₂— in the chain hydrocarbon group included in the hydrocarbon group of L^{b1} and L^{b2} is replaced with —O—, —CO—, —S—, —SO— or —SO₂—, the hydrocarbon group of L^{b1} and L^{b2} can substantially have a substituent such as a hydroxy group, a carboxy group, a carbonyl group, an oxycarbonyl group, a carbonyloxy group, an alkoxy group, an alkylcarbonyl group, an alkoxycarbonyl group, an alkylcarbonyloxy group, a thiol group, an alkylthio group or an alkylsulfonyl group.

The number of substituents that the L^{b1} and the L^{b2} optionally have is not particularly limited, and they optionally have a plurality of substituents.

Examples of the cyclic hydrocarbon group of Y^{b1} include an alicyclic hydrocarbon group, an aromatic hydrocarbon group, and a combined group of an alicyclic hydrocarbon group and an aromatic hydrocarbon group.

Examples of the alicyclic hydrocarbon group and the aromatic hydrocarbon group of Y^{b1} include the same alicyclic hydrocarbon groups and aromatic hydrocarbon groups as those exemplified for L^{b1}, respectively, and when having no substituent, it may be a monovalent alicyclic hydrocarbon group or a monovalent aromatic hydrocarbon group.

Examples of the substituent that the methyl group and the cyclic hydrocarbon group of Y^{b1} optionally have include a halogen atom, a cyano group, a nitro group, and a hydrocarbon group having 1 to 18 carbon atoms and optionally having a halogen atom, a cyano group or a nitro group, where —CH₂— contained in the hydrocarbon group is optionally replaced with —O—, —S—, —CO—, SO— or —SO₂—. The number of carbon atoms of the hydrocarbon group that the cyclic hydrocarbon group of Y^{b1} optionally has is not included in the number of carbon atoms of the cyclic hydrocarbon group of Y^{b1}.

Examples of the hydrocarbon group having 1 to 18 carbon atoms that the methyl group and the cyclic hydrocarbon group of Y^{b1} optionally have as a substituent include a chain hydrocarbon group, an alicyclic hydrocarbon group, an aromatic hydrocarbon group and a combined group of these groups. Examples of the chain hydrocarbon group, the alicyclic hydrocarbon group, the aromatic hydrocarbon group and the combined group of two or more of these groups include the same groups as the chain hydrocarbon groups, alicyclic hydrocarbon groups, aromatic hydrocarbon groups, and combined groups of two or more of these groups exemplified for L^{b1}, within the range permitted by the upper limit of the number of carbon atoms.

In addition, examples of the group formed by replacing —CH₂— contained in the hydrocarbon group having 1 to 18 carbon atoms that the methyl group and the cyclic hydrocarbon group of Y^{b1} optionally have as a substituent with —O—, —S—, CO—, —SO— or —SO₂— include the same groups as those exemplified as the group formed by replacing —CH₂— contained in the hydrocarbon group of L^{b1} with —O—, —S—, —CO—, —SO— or —SO₂—, within the range permitted by the upper limit of the number of carbon atoms. Also, the hydrocarbon group having 1 to 18 carbon atoms that the cyclic hydrocarbon group of Y^{b1} optionally has as a substituent may constitute a protecting group or a leaving group (acid labile group or base labile group), which is commonly used in the field.

It is preferable that the anion of the salt represented by formula (B1) is an anion represented by the following formula (B1-A1) (hereinafter referred to as “anion (B1-A1)” in some cases), or an anion represented by formula (B1-A2) (hereinafter referred to as “anion (B1-A2)” in some cases):

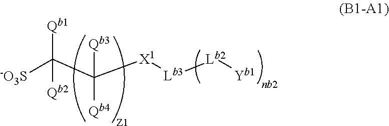

wherein
L^{b2} and Y^{b1} have the same meanings as in formula (B1);
Q^{b1}, Q^{b2}, Q^{b3} and Q^{b4} each independently represent a hydrogen atom, a fluorine atom, a perfluoroalkyl group having 1 to 6 carbon atoms, or an alkyl group having 1 to 6 carbon atoms;
z1 represents an integer of 0 to 6, and when z1 is 2 or larger, a plurality of groups in parentheses may be the same or different from each other;
X¹ represents —O—CO—, —CO—O—, —O—CO—O— or —O—;
L^{b3} represents a single bond or a hydrocarbon group having a valence of (nb2+1), having 1 to 24 carbon atoms, and optionally having a substituent, and —CH₂— contained in the hydrocarbon group is optionally replaced with —O—, —S—, —CO—, —SO— or —SO₂—; and
nb2 represents an integer of 1 to 3, and when nb2 is 2 or larger, a plurality of groups in parentheses may be the same or different from each other.

Examples of the alkyl group of Q^{b1}, Q^{b2}, Q^{b3} and Q^{b4} include a methyl group, an ethyl group, a propyl group, an isopropyl group, a butyl group, a sec-butyl group, a tert-butyl group, a pentyl group and a hexyl group.

Examples of the perfluoroalkyl group of Q^{b1}, Q^{b2}, Q^{b3} and Q^{b4} include a trifluoromethyl group, a perfluoroethyl group, a perfluoropropyl group, a perfluoroisopropyl group, a perfluorobutyl group, a perfluoro-sec-butyl group, a perfluoro-tert-butyl group, a perfluoropentyl group and a perfluorohexyl group.

Q^{b1} and Q^{b2} preferably contain a fluorine atom or a perfluoroalkyl group in at least one of Q^{b1} and Q^{b2}; are more preferably a fluorine atom or a perfluoroalkyl group; are further preferably a fluorine atom or a trifluoromethyl group; and are both further more preferably fluorine atoms.

Q^{b3} and Q^{b4} are each independently preferably a hydrogen atom, a fluorine atom, or a perfluoroalkyl group having 1 to 3 carbon atoms; Q^{b3} is preferably a hydrogen atom, a fluorine atom, or a perfluoroalkyl group having 1 to 3 carbon atoms; and Q^{b4} is preferably a hydrogen atom or a fluorine atom.

z1 is preferably an integer of 0 to 3, and is more preferably 0, 1 or 2.

X¹ is preferably —O—CO— or —CO—O—.

Examples of the hydrocarbon group in L^{b3} include the same hydrocarbon groups as those exemplified for L^{b1} in formula (B1), within the range permitted by the upper limit of the number of carbon atoms.

L^{b3} is preferably a single bond, or a chain hydrocarbon group having 1 to 12 carbon atoms and optionally having a substituent, where —CH₂— contained in the chain hydrocarbon group is optionally replaced with —O— or —CO—, an alicyclic hydrocarbon group having 3 to 18 carbon atoms and optionally having a substituent, where —CH₂— contained in the alicyclic hydrocarbon group is optionally replaced with —O—, —S—, —CO—, —SO— or —SO₂—, an aromatic hydrocarbon group having 6 to 10 carbon atoms and optionally having a substituent, where —CH₂— contained in the aromatic hydrocarbon group is optionally replaced with —O—or —S—, or a combined group of two or more of these groups; and is more preferably a single bond, a chain hydrocarbon group having 1 to 6 carbon atoms, or a group represented by the following formula (Lb3-1). In addition, when —CH₂— contained in the chain hydrocarbon group is replaced with —O— or —CO—, the number of —CH₂— contained in the chain hydrocarbon group replaced with —O— or —CO— is preferably 1 to 4; and it is preferable that one —CH₂—CH₂— contained in the chain hydrocarbon group is replaced with —O—CO— or —CO—O—, or one —CH₂—CH₂—CH₂— contained in the chain hydrocarbon group is replaced with —O—CO—O—:

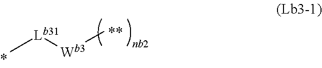

wherein
nb2 has the same meaning as in formula (B1-A1);
L^{b31} represents a single bond or a chain hydrocarbon group having 1 to 12 carbon atoms, —CH₂— contained in the chain hydrocarbon group is optionally replaced with —O— or —CO—, and the chain hydrocarbon group optionally has a substituent;
W^{b3} represents an alicyclic hydrocarbon group having 3 to 18 carbon atoms or an aromatic hydrocarbon group having 6 to 10 carbon atoms, —CH₂-contained in the alicyclic hydrocarbon group is optionally replaced with —O—, —S, —CO—, —SO— or —SO₂—, —CH₂— contained in the aromatic hydrocarbon group is optionally replaced with —O— or —S—, and the alicyclic hydrocarbon group and the aromatic hydrocarbon group optionally have a substituent; and
* and ** represent bonding sites, and * represents a bonding site to X¹.

In formula (Lb3-1), examples of the chain hydrocarbon group of Lb3l include the same chain hydrocarbon groups as those exemplified for L^{b1}, within the range permitted by the upper limit of the number of carbon atoms.

In formula (Lb3-1), examples of the alicyclic hydrocarbon group and the aromatic hydrocarbon group of W^{b3} include the same alicyclic hydrocarbon groups and aromatic hydrocarbon groups as those exemplified for L^{b1}, within the range permitted by the upper limit of the number of carbon atoms.

In formula (Lb3-1), examples of the substituent that the chain hydrocarbon group of L^{b31} optionally has and the substituent that the alicyclic hydrocarbon group and the aromatic hydrocarbon group of W^{b3} optionally have include the same substituents as those exemplified as the substituent that the hydrocarbon group of L^{b1} optionally has.

It is preferable that L^{b31} is a single bond or an alkanediyl group having 1 to 6 carbon atoms, where —CH₂— contained in the alkanediyl group is optionally replaced with —O— or —CO—.

Examples of the alicyclic hydrocarbon group and the aromatic hydrocarbon group of W^{b3} include the following alicyclic hydrocarbon groups and aromatic hydrocarbon groups. In the alicyclic hydrocarbon groups and aromatic hydrocarbon groups, * and ** represent bonding sites, * represents a bonding site to X¹ or L^{b31}** represents a hydrogen atom, a substituent or a bonding site to L^{b2}, and at least one ** represents a bonding site to L^{b2}. In addition, in the alicyclic hydrocarbon groups represented below, —CH₂— contained in the alicyclic hydrocarbon group is optionally replaced with —O—, —S, —CO—, —SO— or —SO₂—. When —CH₂— contained in the alicyclic hydrocarbon group is replaced with —O—, —S—, —CO—, —SO— or —SO₂—, it is preferable that an ether ring, an ester ring (lactone ring), a carbonate ester ring, a sulfonate ester ring (sultone ring) or an acetal ring is formed.

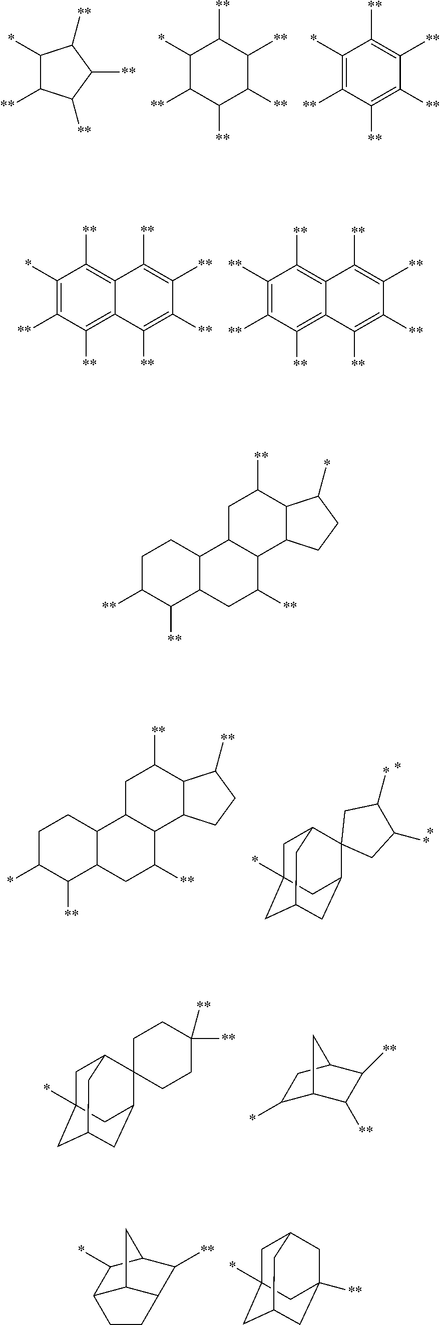

In formula (B1-A1), L^{b2} is preferably a single bond or a chain hydrocarbon group having 1 to 12 carbon atoms, where —CH₂— contained in the chain hydrocarbon group is optionally replaced with —O— or —CO—, and is more preferably a single bond, —O—, —O—CO—, —CO—O—, —O—CO—O— or *-L^{b21}-X²-L^{b22}-**, where any one of L^{b21} and L^{b22} represents a chain hydrocarbon group having 1 to 6 carbon atoms, and the other represents a single bond or a chain hydrocarbon group having 1 to 6 carbon atoms; X² represents —O—, —CO—O—, —O—CO— or —O—CO—O—; * and ** represent bonding sites and ** represents a bonding site to Y^{b1}; and the total number of carbon atoms of L^{b2}, X² and L^{b22} is 12 or less.

In formula (B1-A1), Y^{b1} is preferably a cyclic hydrocarbon group having 3 to 20 carbon atoms and optionally having a substituent, where —CH₂— contained in the cyclic hydrocarbon group is optionally replaced with —O—, —CO—, —S—, —SO— or —SO₂—, is more preferably an alicyclic hydrocarbon group having 3 to 18 carbon atoms and optionally having a substituent, where —CH₂— contained in the alicyclic hydrocarbon group is optionally replaced with —O—, —CO—, —S—, —SO— or —SO₂—, or an aromatic hydrocarbon group having 6 to 18 carbon atoms and optionally having a substituent, and is further preferably an alicyclic hydrocarbon group having 3 to 16 carbon atoms and optionally having a substituent, where —CH₂— contained in the alicyclic hydrocarbon group is optionally replaced with —O—, —CO—, —S—, —SO— or —SO₂—, or an aromatic hydrocarbon group having 6 to 10 carbon atoms and optionally having a substituent. Specifically, it is more preferably a group represented by any one of the following formulas (Y1) to (Y36). In formulas (Y1) to (Y36), R^{Yb} represents a hydrogen atom or an alkyl group having 1 to 4 carbon atoms among the substituents that the cyclic hydrocarbon group of Y^{b1} optionally has, R^{Yc} represents a hydrogen atom or a substituent that the cyclic hydrocarbon group of Y^{b1} optionally has, and * represents a bonding site to L^{b2}. The alicyclic hydrocarbon groups and the aromatic hydrocarbon groups represented by the following formulas optionally have other substituents although not particularly shown in the following formulas.

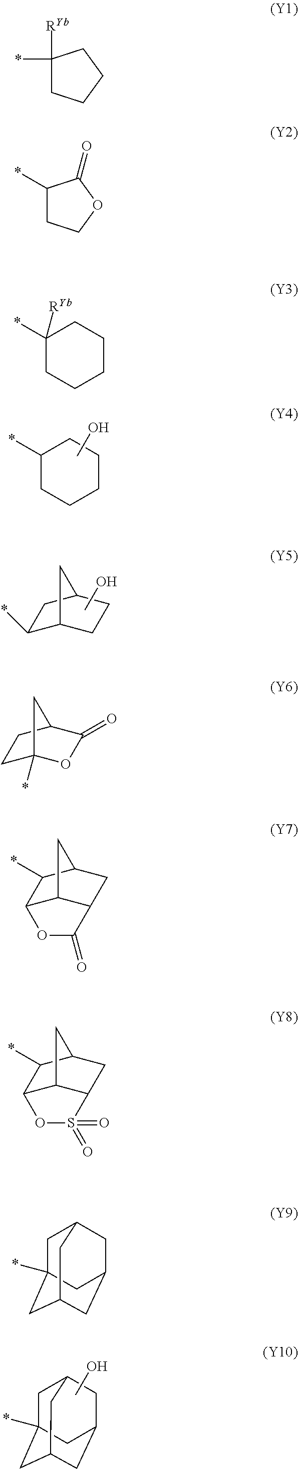

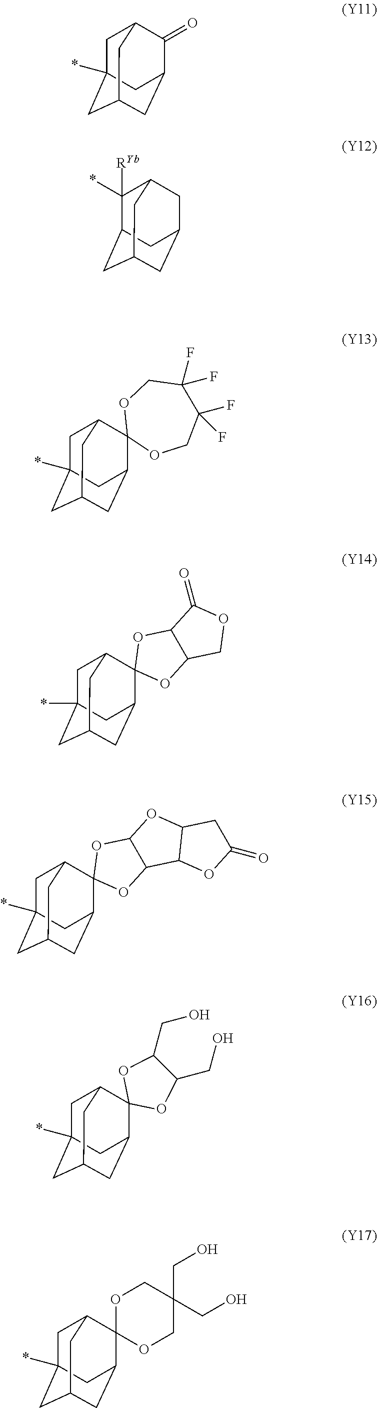

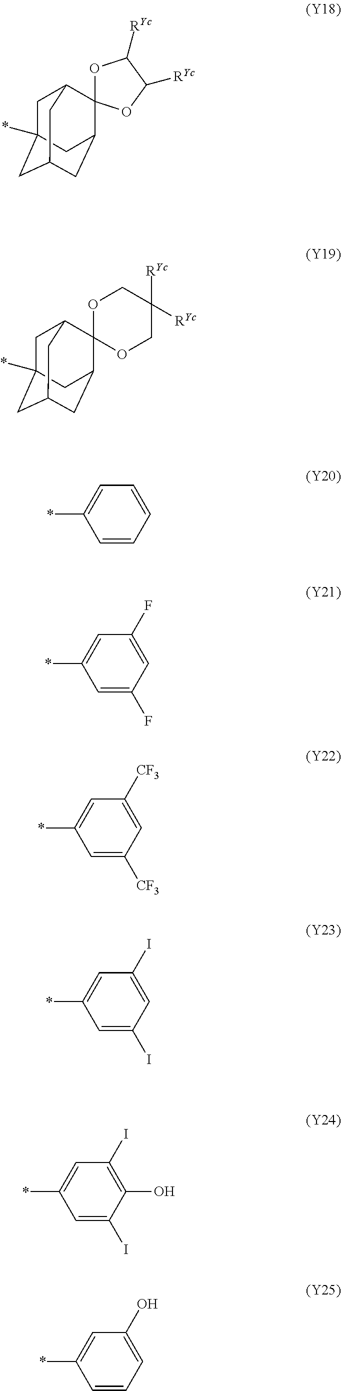

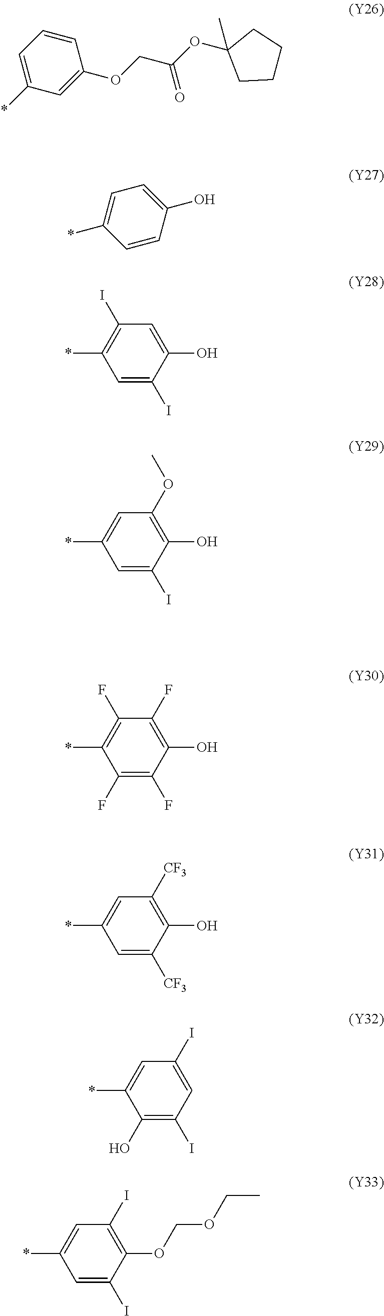

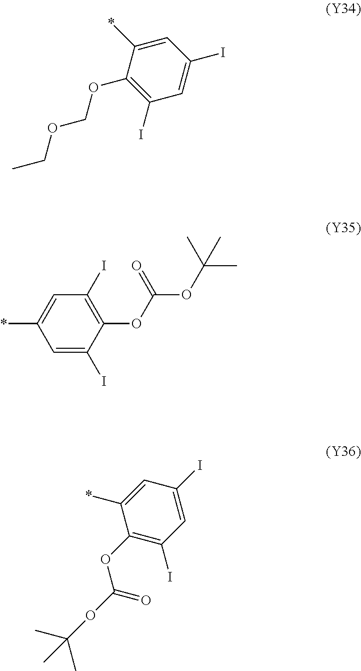

The anion represented by formula (B1-A1) is preferably an anion represented by any one of formulas (B1-A1-1) to (B1-A1-85) (hereinafter referred to as “anion (B1-A1-1)” or the like according to the formula number in some cases), and is more preferably an anion represented by any one of formulas (B1-A1-1) to (B1-A1-4), (B1-A1-9), (B1-A1-10), (B1-A1-24) to (B1-A1-33), (B1-A1-36) to (B1-A1-40), and (B1-A1-47) to (B1-A1-85).

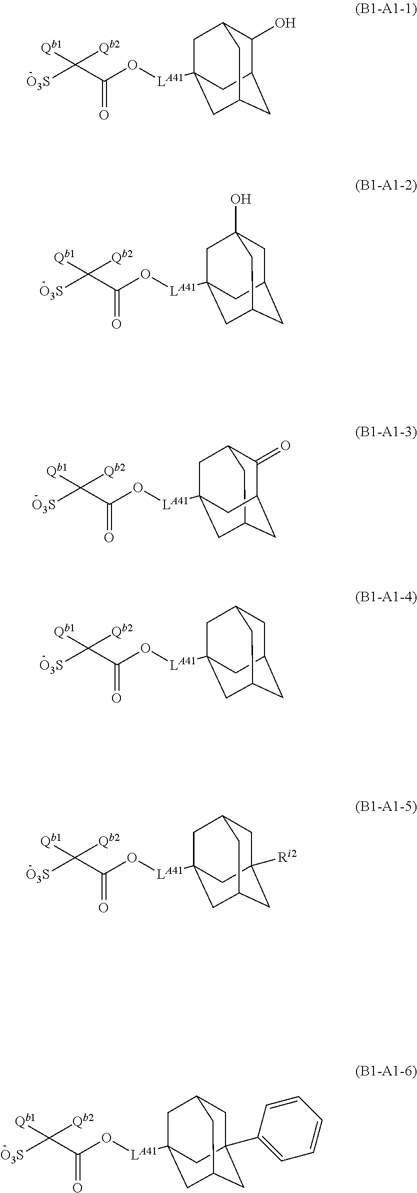

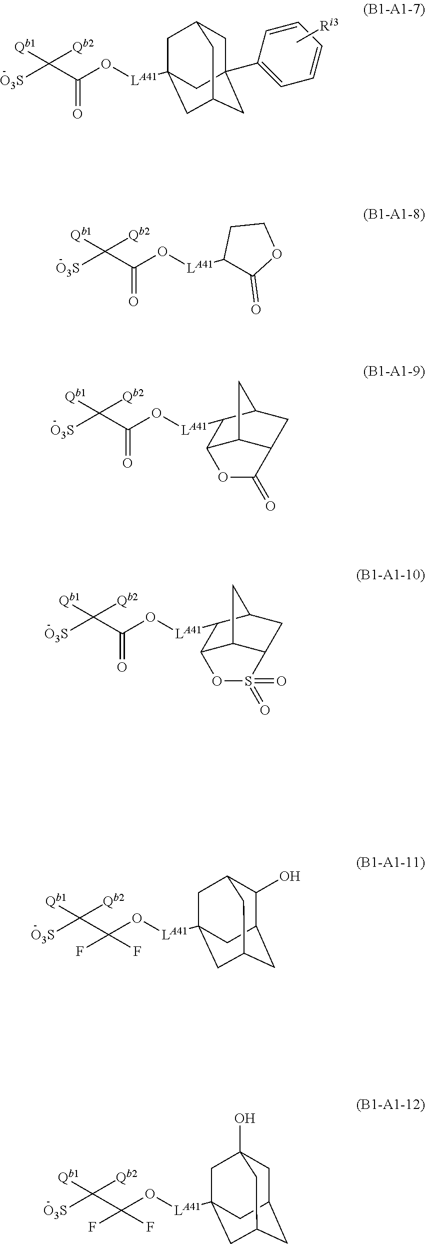

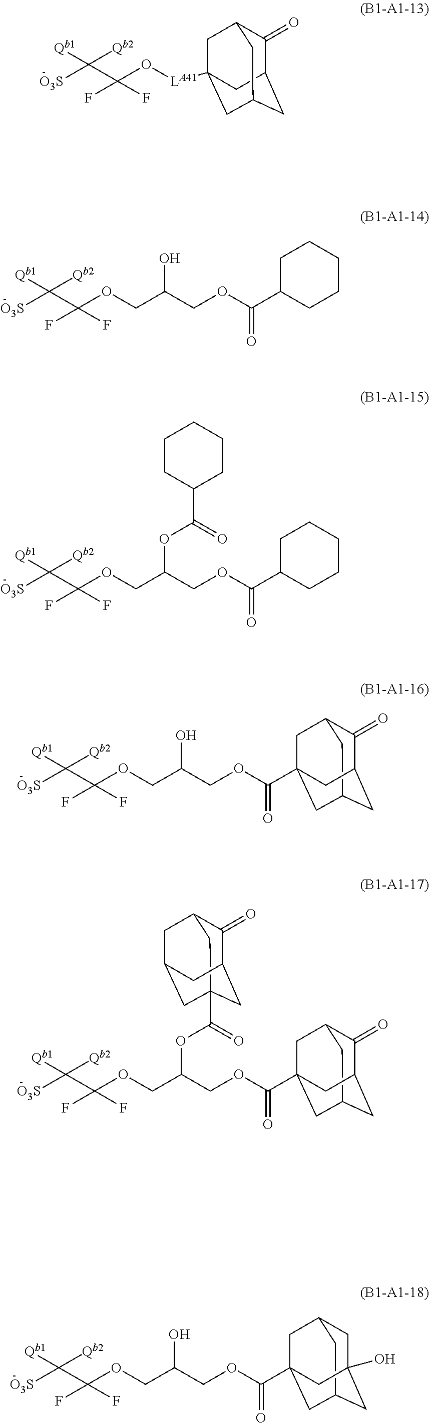

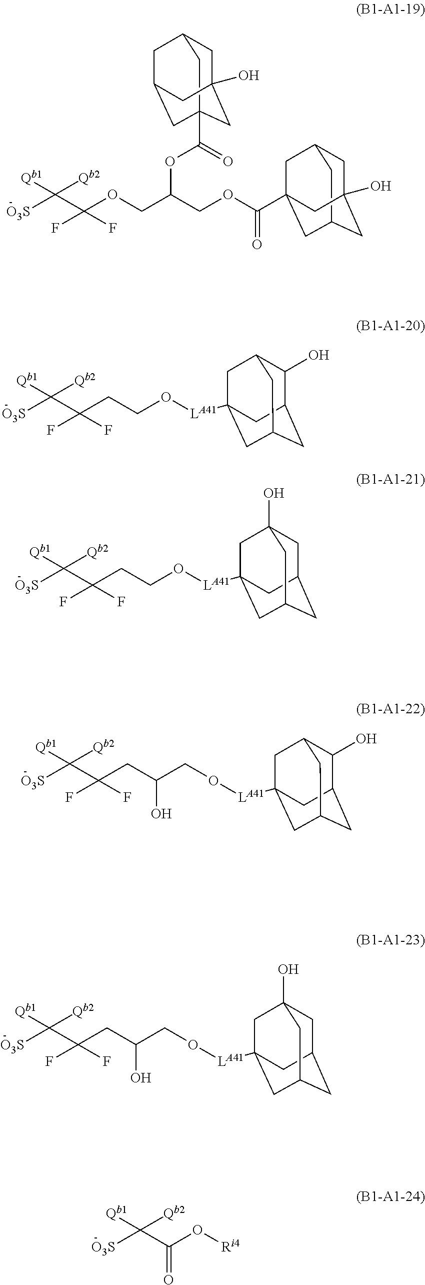

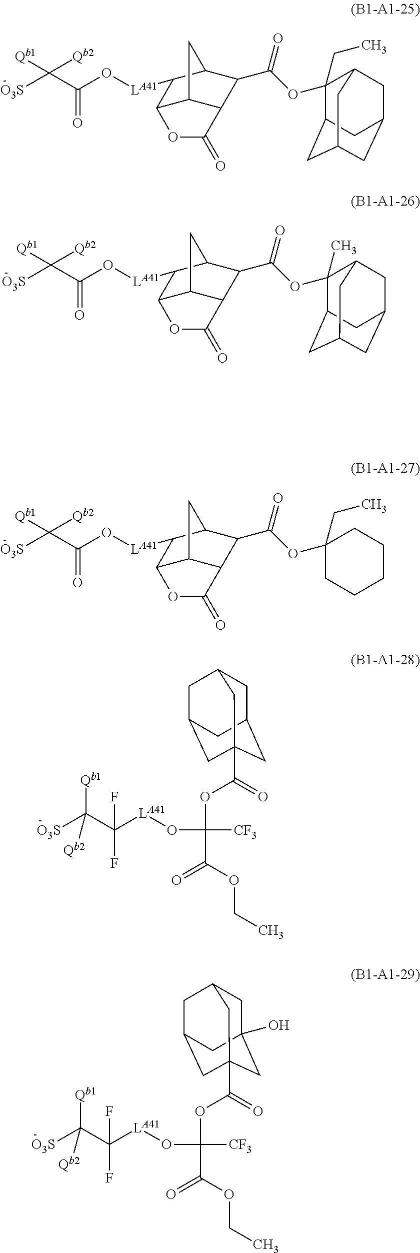

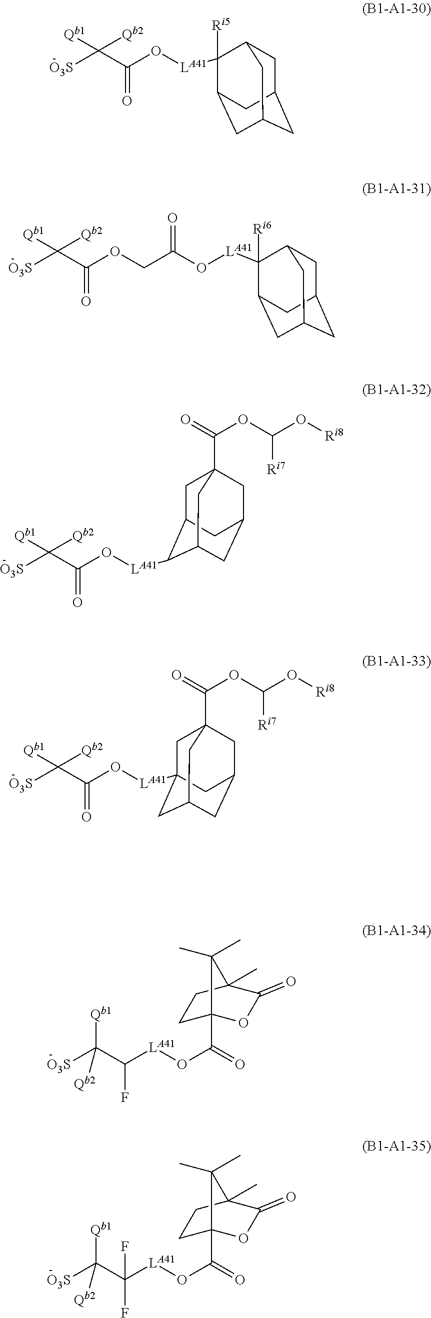

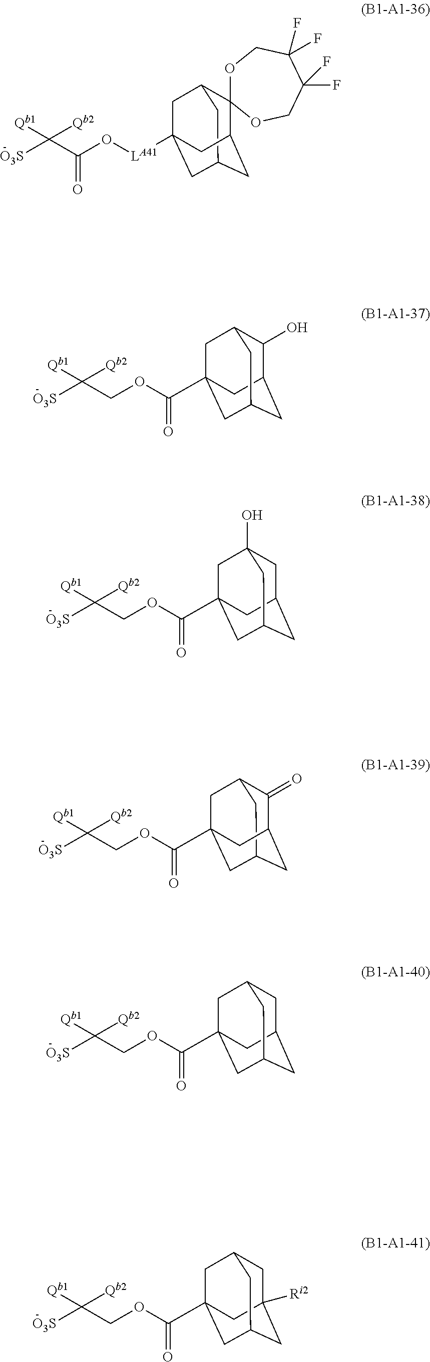

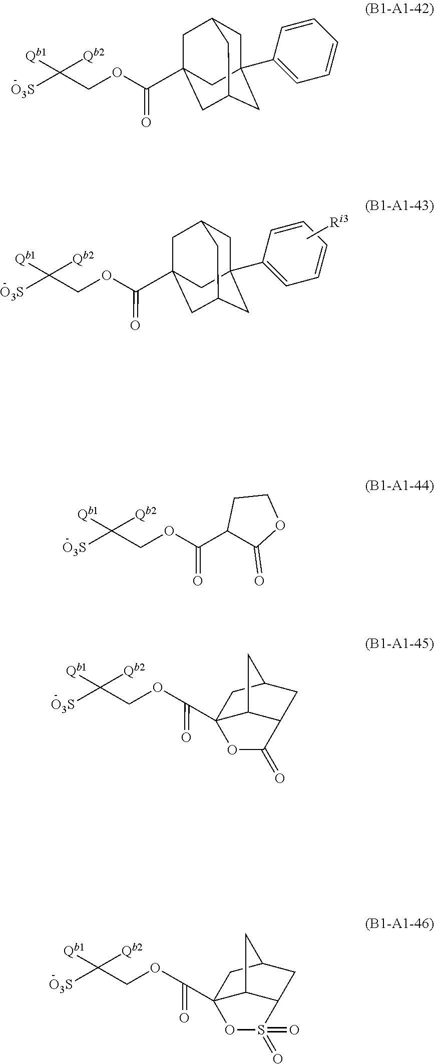

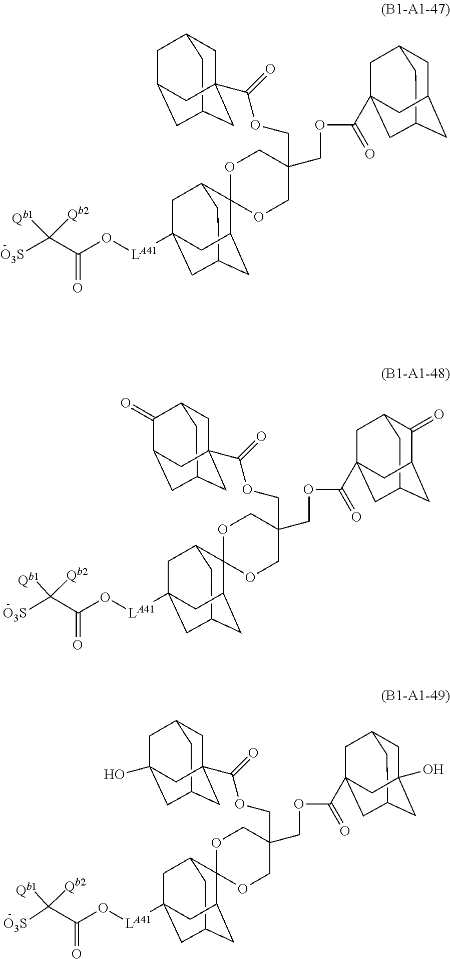

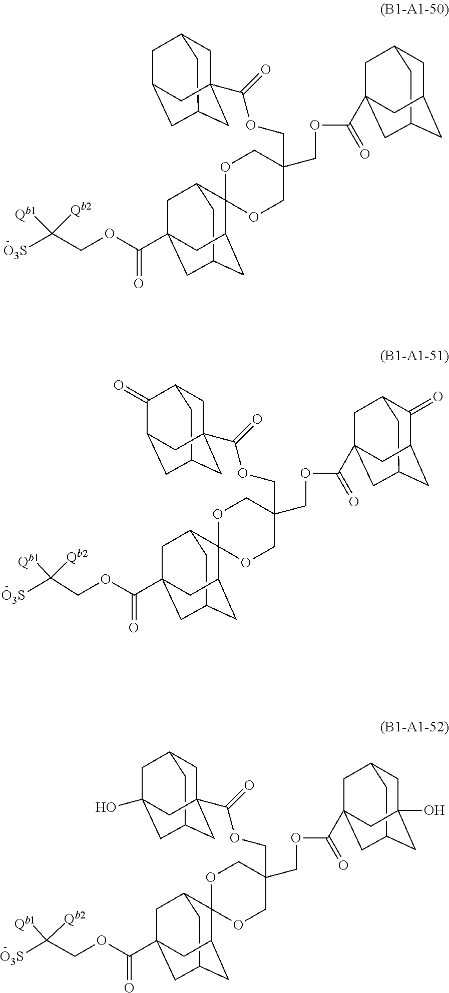

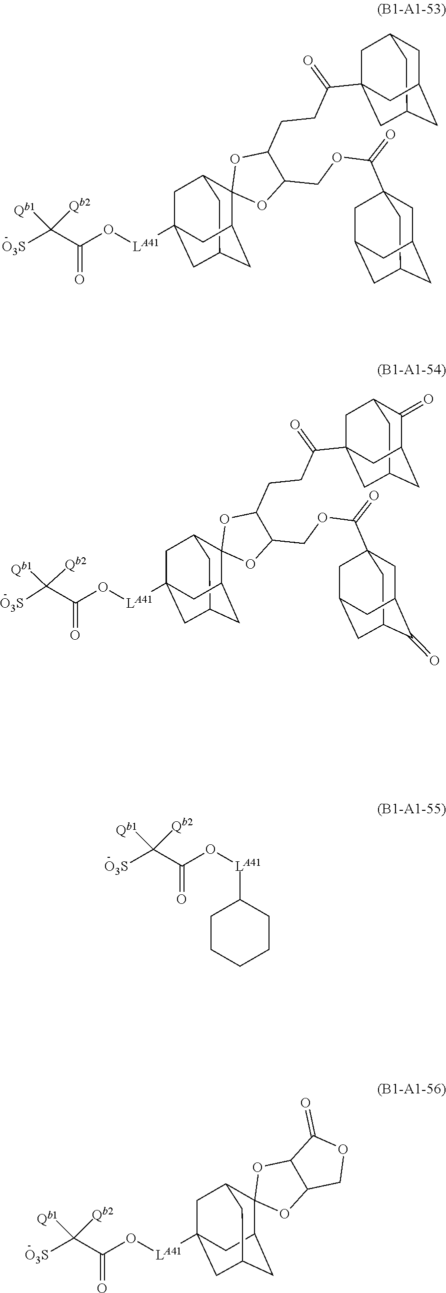

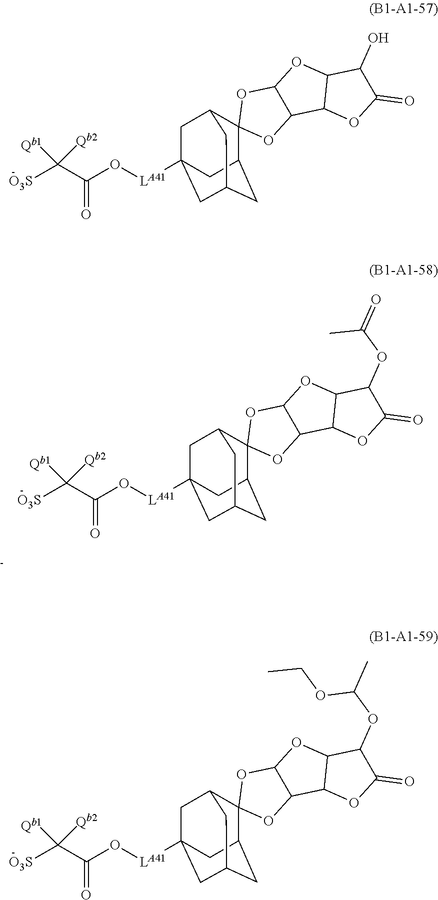

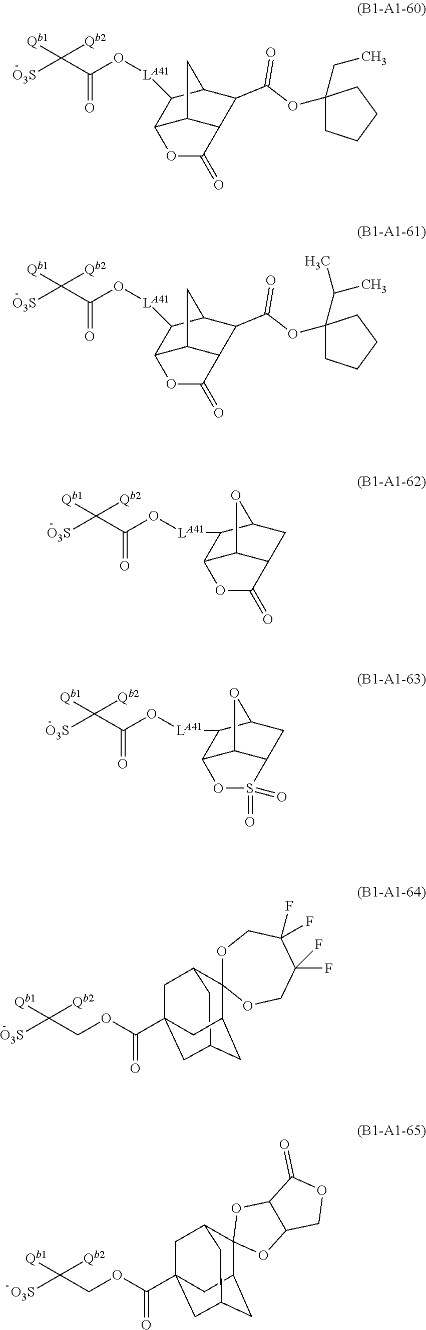

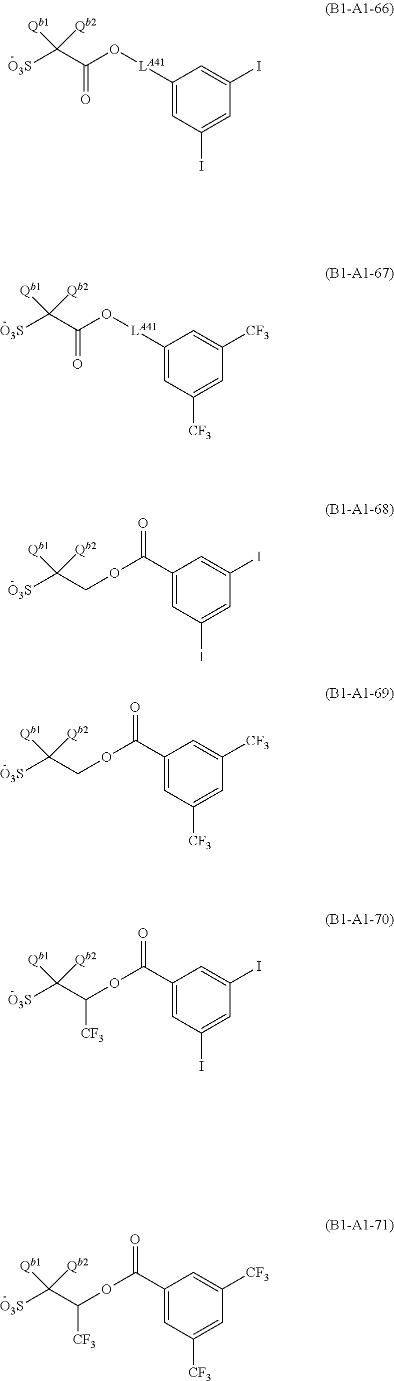

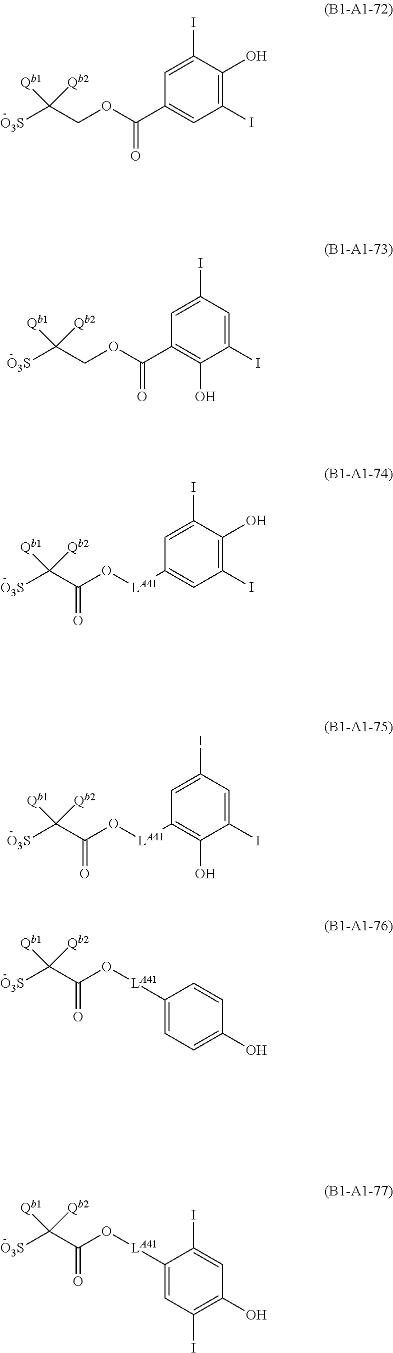

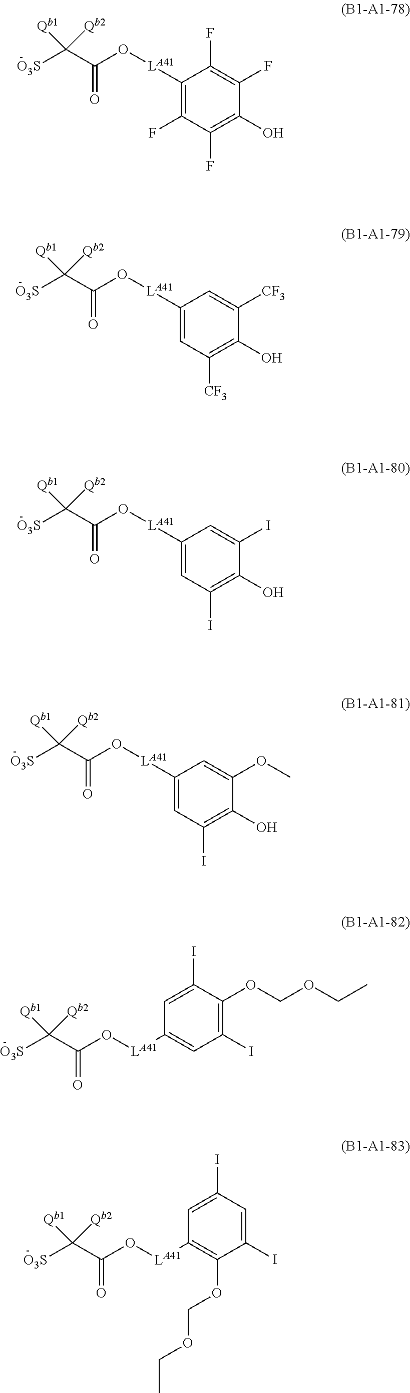

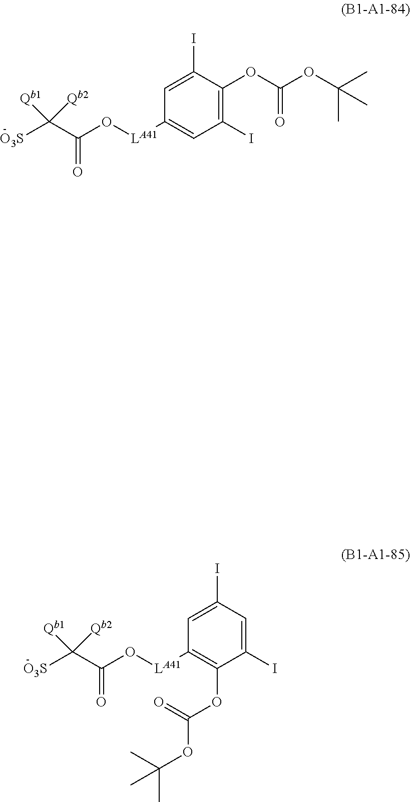

Here, Rⁱ² to Rⁱ⁷ are each independently an alkyl group having 1 to 4 carbon atoms, for example, and is preferably a methyl group or an ethyl group. Rⁱ⁸ is, for example, an aliphatic hydrocarbon group having 1 to 12 carbon atoms, is preferably an alkyl group having 1 to 4 carbon atoms, an alicyclic hydrocarbon group having 5 to 12 carbon atoms, or a group formed by combining these groups, and is more preferably a methyl group, an ethyl group, a cyclohexyl group or an adamantyl group. L^{A41} is a single bond or an alkanediyl group having 1 to 4 carbon atoms.

Q^{b1} and Q^{b2} have the same meanings as described above.

Specific examples of the anion represented by formula (B1-A1) include the anions disclosed in Japanese Patent Laid-Open No. 2010-204646.

Preferred examples of the anion represented by formula (B1-A1) include the respective anions represented by formulas (B1a-1) to (B1a-82). In particular, an anion is preferable that is represented by any one of formulas (B1a-1) to (B1a-4), (B1a-7) to (B1a-11), (B1a-14) to (B1a-30), and (B1a-35) to (B1a-72).

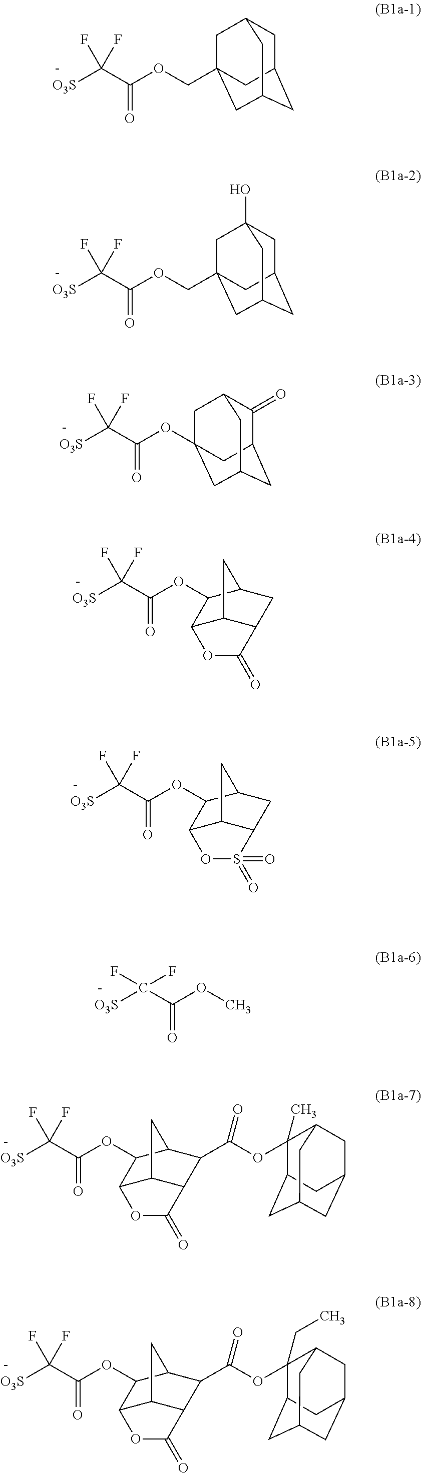

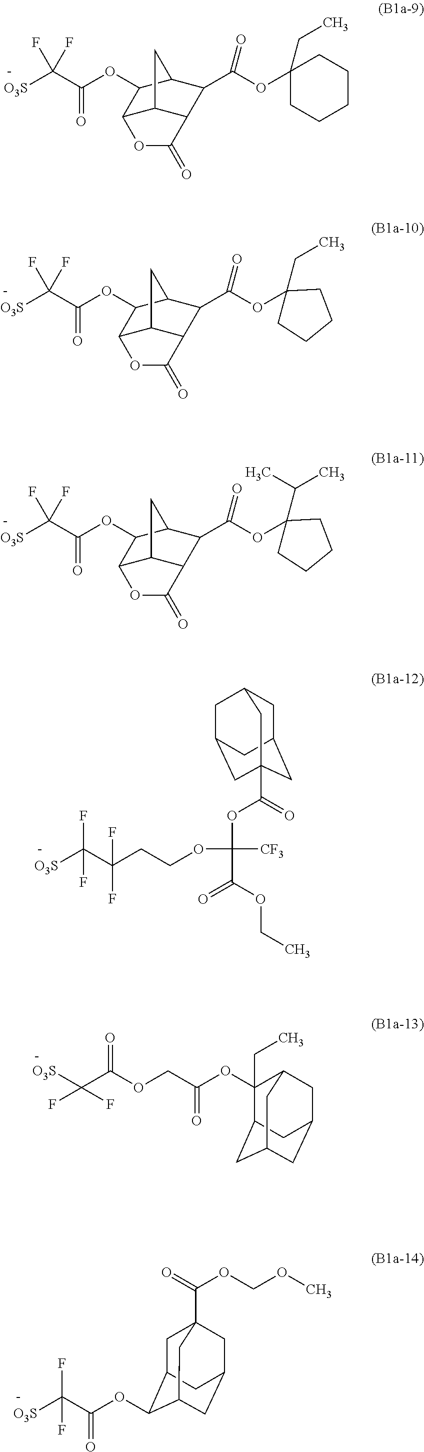

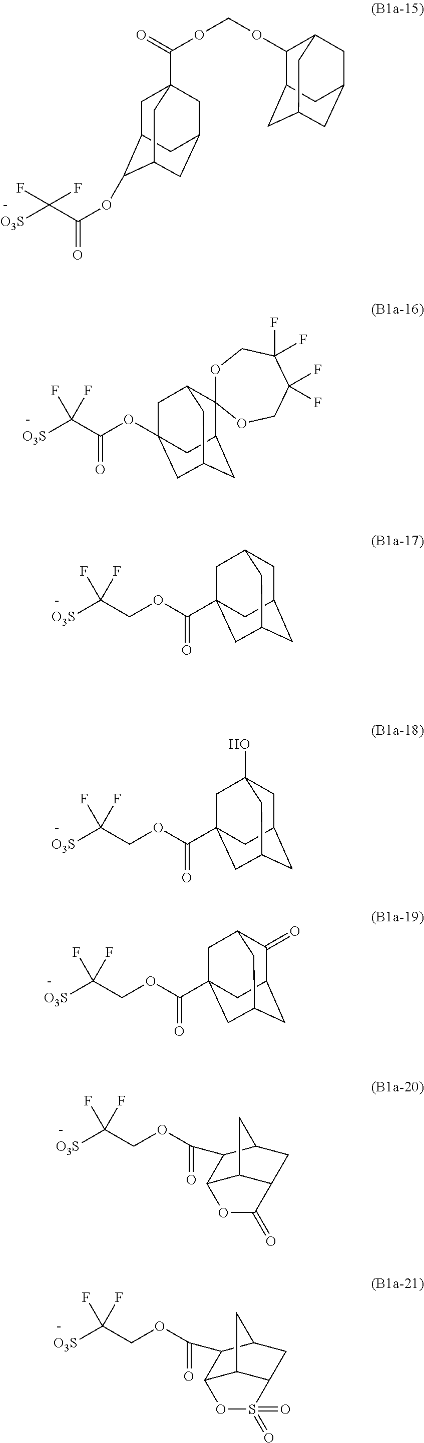

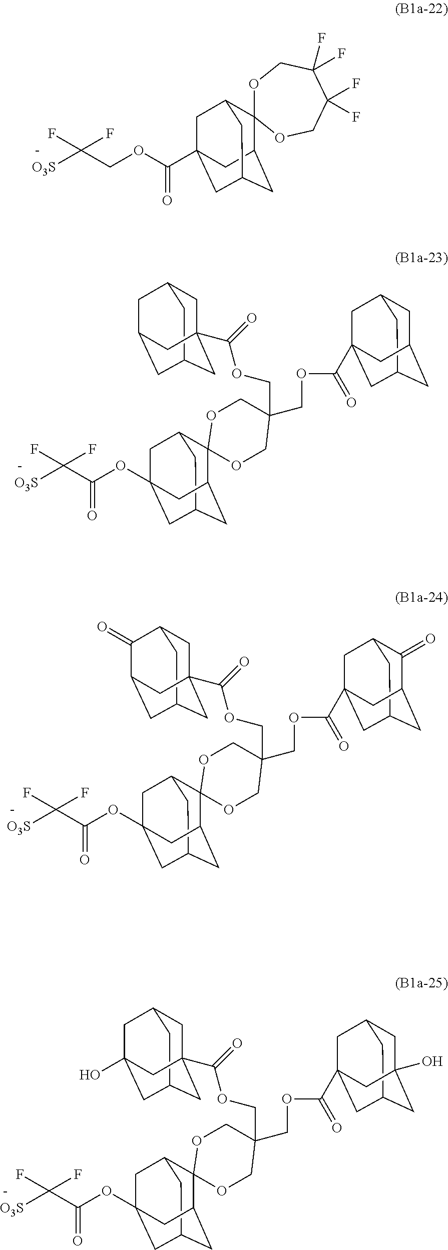

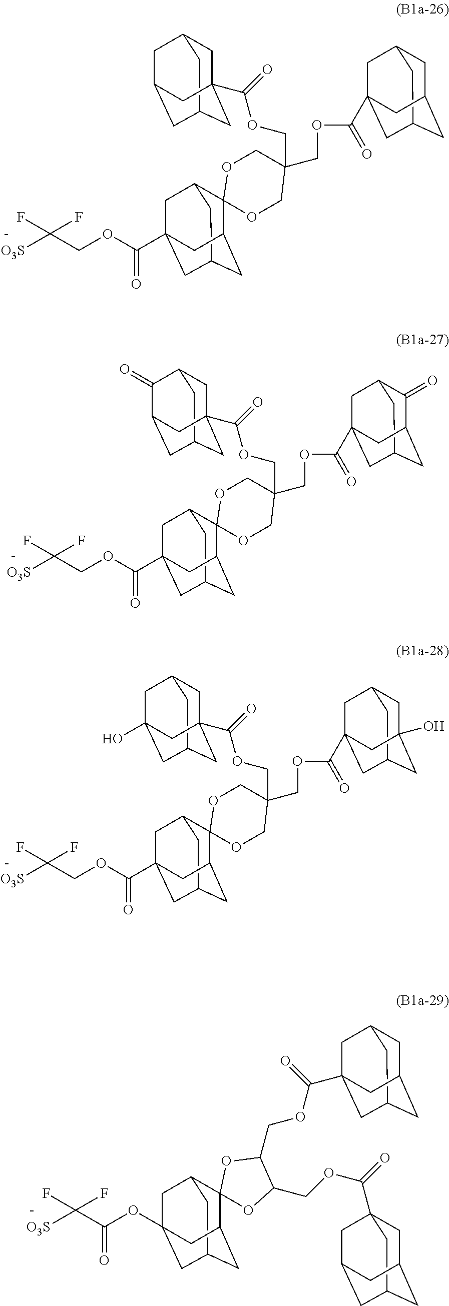

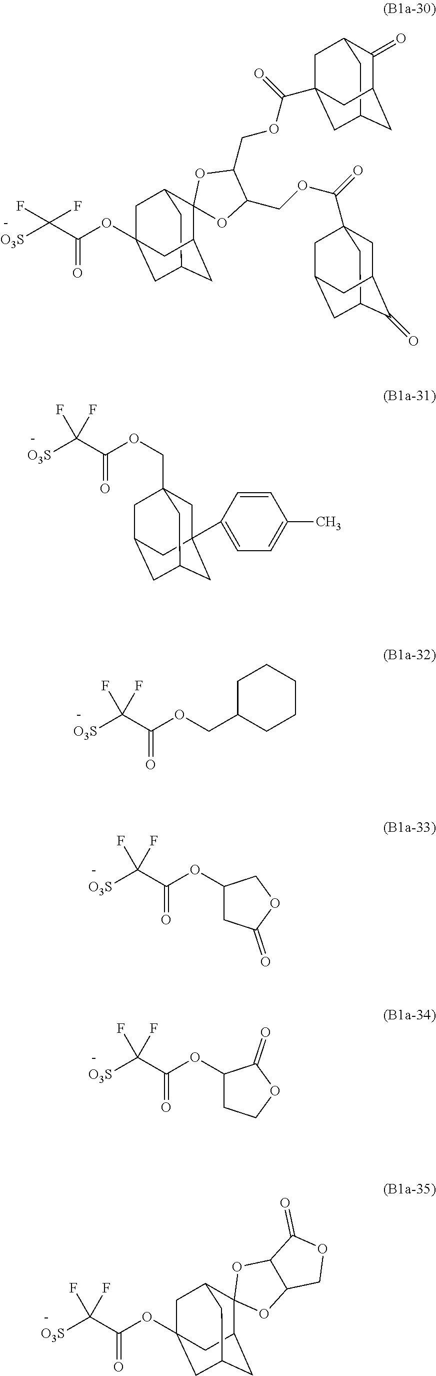

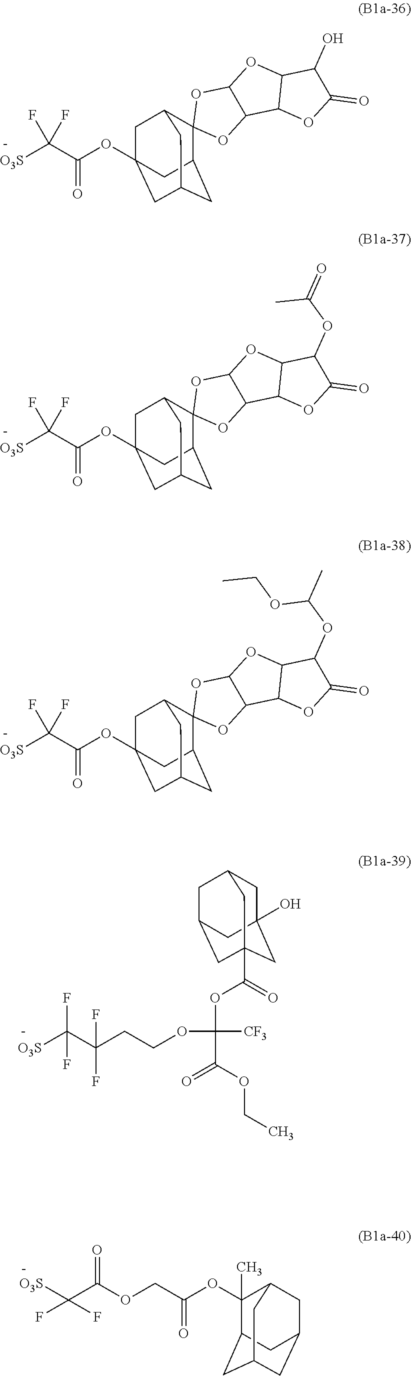

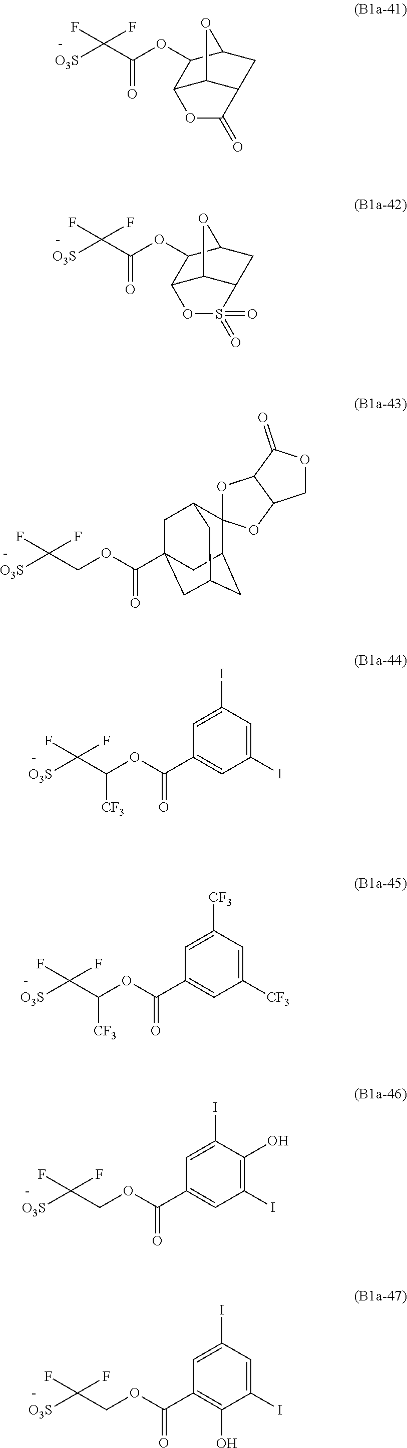

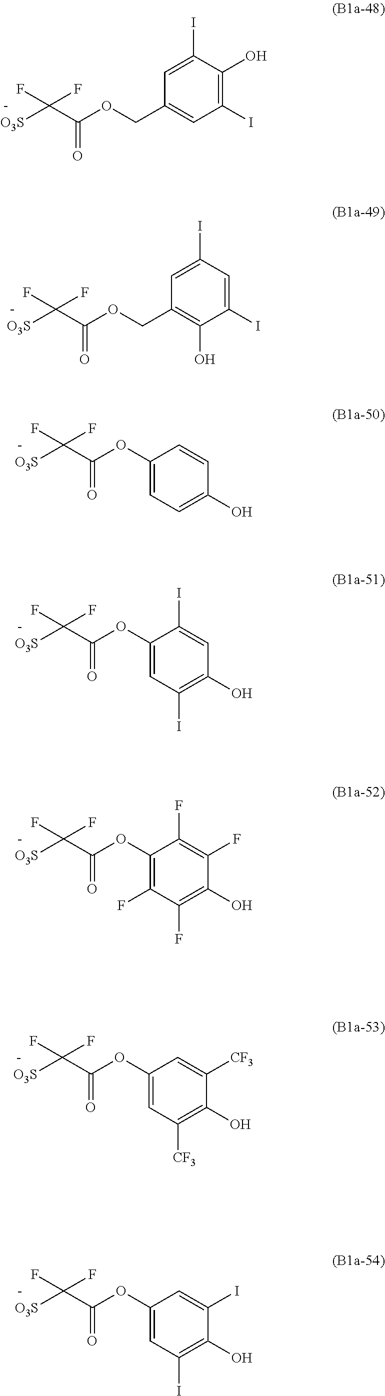

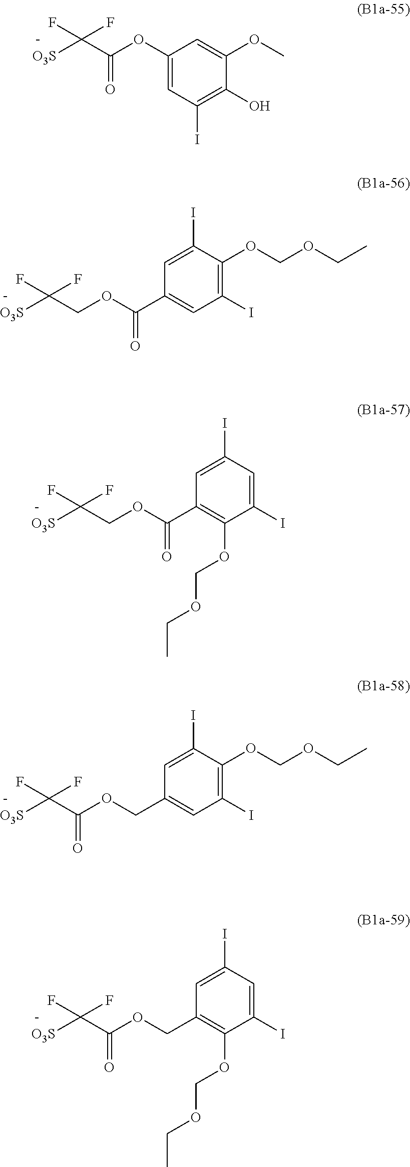

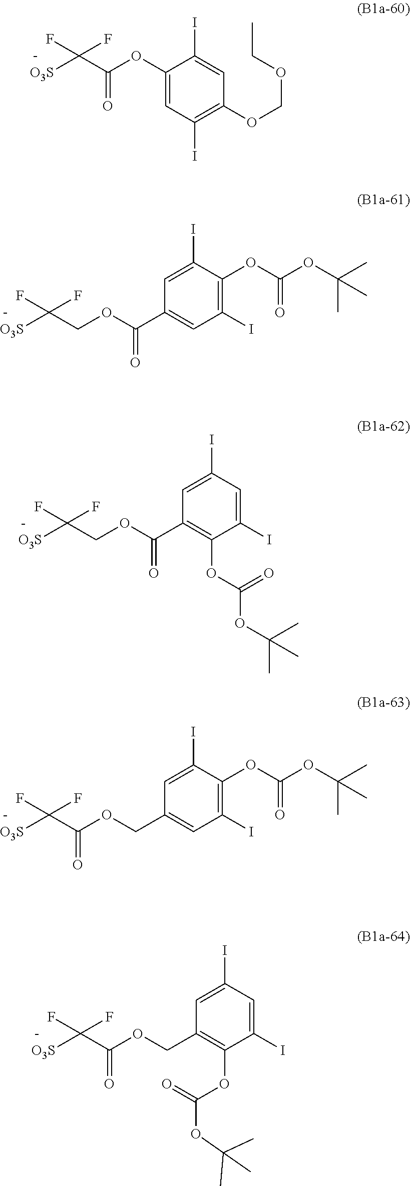

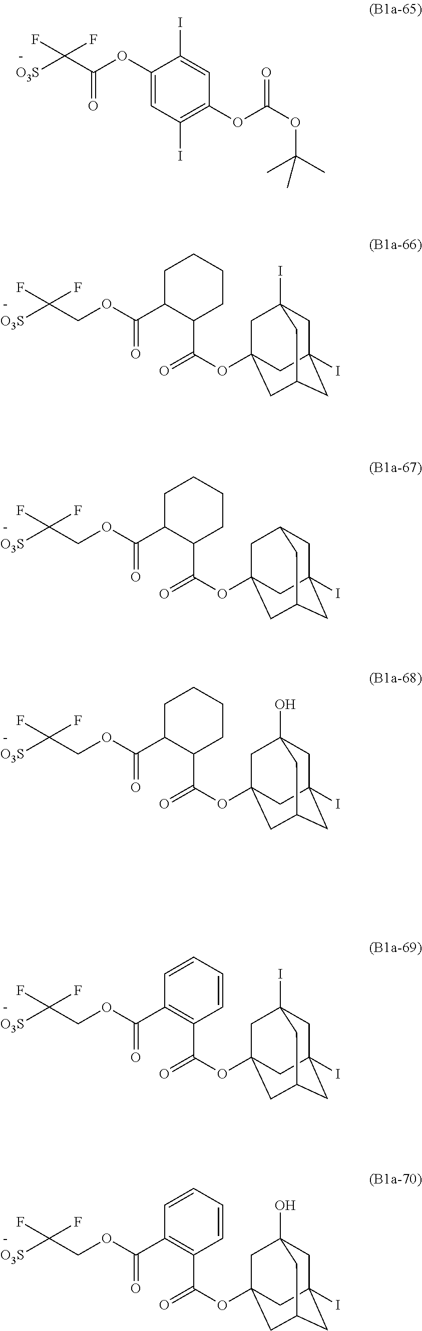

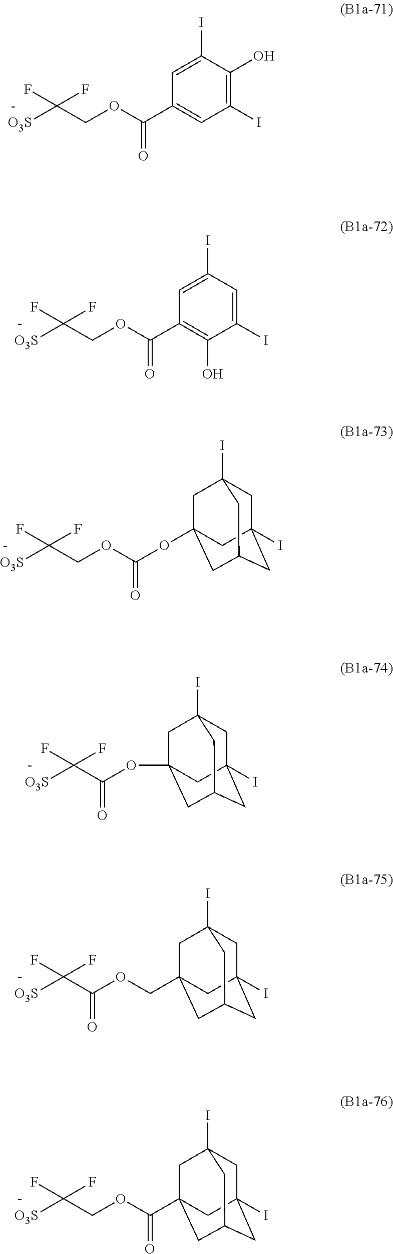

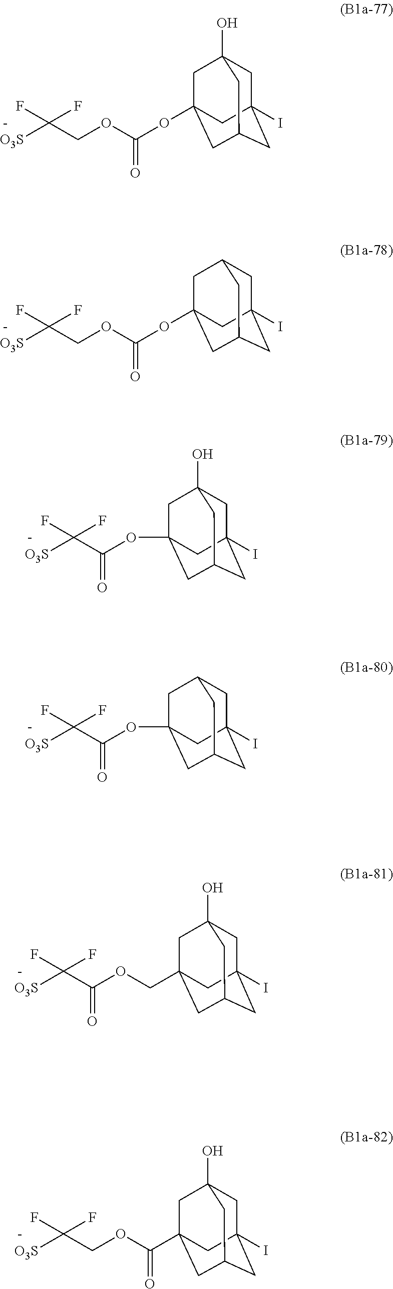

The anion represented by formula (B1-A2) is represented by the following formula:

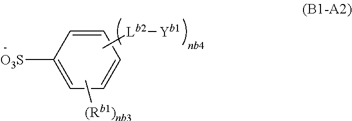

wherein
L^{b2} and Y^{b1} have the same meanings as in formula (B1);
R^{b1} represents a halogen atom or an alkyl group having 1 to 6 carbon atoms, and —CH₂— contained in the alkyl group is optionally replaced with —O— or —CO—;
nb4 represents an integer of 1 to 5, and when nb4 is 2 or larger, a plurality of groups in parentheses may be the same or different from each other; and
nb3 represents an integer of 0 to 4, and when nb3 is 2 or larger, a plurality of R^{b1} may be the same or different from each other,
provided that 1≤nb4+nb3≤5 is satisfied.

In formula (B1-A2), examples of the hydrocarbon group of L^{b2} and examples of the cyclic hydrocarbon group of Y^{b1} include the same groups as in the examples of the hydrocarbon group of L^{b2} and the examples of the cyclic hydrocarbon group of Y^{b1} in formula (B1), within the range permitted by the upper limit of the number of carbon atoms. Examples of the substituent that the hydrocarbon group of L^{b2}, and the methyl group and the cyclic hydrocarbon group of Y^{b1} optionally have also include the same as those exemplified as the substituent that the hydrocarbon group of L^{b2}, and the methyl group and the cyclic hydrocarbon group of Y^{b1} in formula (B1) optionally have.

The hydrocarbon group, methyl group and cyclic hydrocarbon group optionally have one substituent or a plurality of substituents.

L^{b2} is preferably a chain hydrocarbon group having 1 to 12 carbon atoms, where —CH₂— contained in the chain hydrocarbon group is optionally replaced with —O— or —CO—, is more preferably *—CO—O-L^{b41}- where L^{b41} is a single bond or a chain hydrocarbon group of 1 to 6 carbon atoms, —CH₂— contained in the chain hydrocarbon group is optionally replaced with —O— or —CO—, * represents a bonding site to a benzene ring to which SO₃⁻ is bonded, and L^{b41} is preferably a single bond or a chain hydrocarbon group having 1 to 3 carbon atoms, and is further more preferably *—CO—O—.

Y^{b1} is preferably a cyclic hydrocarbon group having 3 to 20 carbon atoms and optionally having a substituent, where —CH₂— contained in the cyclic hydrocarbon group is optionally replaced with —O—, —CO—, —S—, —SO— or —SO₂—, is more preferably an alicyclic hydrocarbon group having 3 to 18 carbon atoms and optionally having a substituent, where —CH₂— contained in the alicyclic hydrocarbon group is optionally replaced with —O—, —CO—, —S—, —SO— or —SO₂—, or an aromatic hydrocarbon group having 6 to 18 carbon atoms and optionally having a substituent, and is further preferably the alicyclic hydrocarbon group or aromatic hydrocarbon group exemplified for Y^{b1} and L^{b1} in formula (B1-A1). Specifically, it is preferably a group represented by any one of the above formulas (Y1) to (Y36), and is more preferably a group represented by any one of the above formulas (Y1) to (Y19).

The substituent that the cyclic hydrocarbon group of Y^{b1} optionally has is preferably a halogen atom, or an alkyl group having 1 to 6 carbon atoms, where —CH₂— contained in the alkyl group is optionally replaced with —O—, —S—, —CO—, —SO— or —SO₂—, is more preferably a fluorine atom, an iodine atom, or an alkyl group having 1 to 4 carbon atoms, where —CH₂— contained in the alkyl group is optionally replaced with —O—, —S—, —CO—, —SO— or —SO₂—, is further preferably a fluorine atom, an iodine atom, a hydroxy group, an alkoxy group having 1 to 3 carbon atoms, or an alkyl group having 1 to 4 carbon atoms, and is still more preferably a fluorine atom, an iodine atom, a hydroxy group, or an alkyl group having 1 to 4 carbon atoms. Also, the substituent of Y^{b1} may constitute a protecting group or a leaving group (acid labile group or base labile group), which is commonly used in the field.

In formula (B1-A2), nb4 is preferably an integer of 1 to 4, is more preferably an integer of 1 to 3, is further preferably 1 or 2, and is further more preferably 2. When nb4 is 1 or 2, the following structures are preferable in which the bonding position of -L^{b2}-Y^{b1} is the m-position substitution on the benzene ring with respect to the bonding position of SO₃⁻:

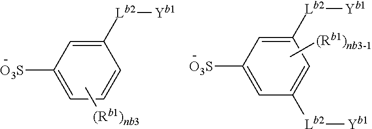

wherein L^{b2}, Y^{b1}, R^{b1} and nb3 have the same meanings as in formula (B1-A2).

In formula (B1-A2), in a case where nb4 is 2 or larger, it is preferable that a plurality of L^{b2} and Y^{b1} are each the same group.

Examples of the alkyl group having 1 to 6 carbon atoms of R^{b1} include an alkyl group such as a methyl group, an ethyl group, a propyl group, an isopropyl group, a butyl group, an isobutyl group, a tert-butyl group, a pentyl group and a hexyl group. The number of carbon atoms of the alkyl group is preferably 1 to 4, and is more preferably 1 to 3.

Examples of the halogen atom of R^{b1} include a fluorine atom, a chlorine atom, a bromine atom and an iodine atom.

Examples of the group formed by replacing —CH₂— contained in the alkyl group with —O— or —CO— include a hydroxy group, a carboxy group, an alkoxy group, an alkoxycarbonyl group, an alkylcarbonyl group, an alkylcarbonyloxy group, an oxy group and a carbonyl group. Examples of these groups formed by the replacement include the same groups as those exemplified in the present specification, within the range permitted by the upper limit of the number of carbon atoms.

R is preferably a halogen atom or an alkyl group having 1 to 4 carbon atoms, where —CH₂— contained in the alkyl group is optionally replaced with —O— or —CO—, is more preferably a fluorine atom, an iodine atom or an alkyl group having 1 to 3 carbon atoms, where —CH₂— contained in the alkyl group is optionally replaced with —O— or —CO—, and is further preferably a fluorine atom, an iodine atom, a hydroxy group, a methoxy group or a methyl group.

nb3 is an integer of 0 to 4 (that is, nb3−1 is an integer of 0 to 3), is preferably an integer of 0 to 3, and is more preferably an integer of 0 to 2. In one embodiment, nb3 is preferably 0. In another embodiment, nb3 is preferably 1 or 2. When nb3 is 1, R^{b1} is preferably a halogen atom, and R^{b1} is more preferably a fluorine atom or an iodine atom. When nb3 is 2, it is preferable that one of R^{b1} is a halogen atom and the other is a halogen atom or an alkyl group having 1 to 4 carbon atoms, and it is more preferable that one of R^{b1} is a fluorine atom or an iodine atom and the other is a fluorine atom, an iodine atom or an alkyl group having 1 to 3 carbon atoms.

Examples of the anion represented by formula (B1-A2) include the following anions. Among these anions, the anions represented by formulas (B2a-1) to (B2a-32) are preferable, and the anions represented by formulas (B2a-1) to (B2a-11) and (B2a-16) to (B2a-32) are more preferable. In the following anions, a part of R^{b1} is omitted, and the anions may contain substituents which are not illustrated.

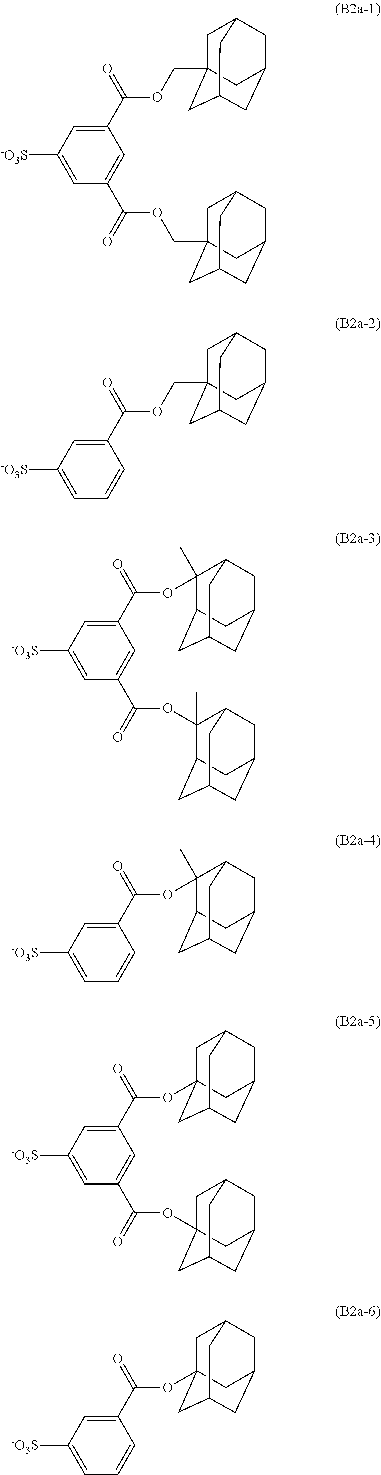

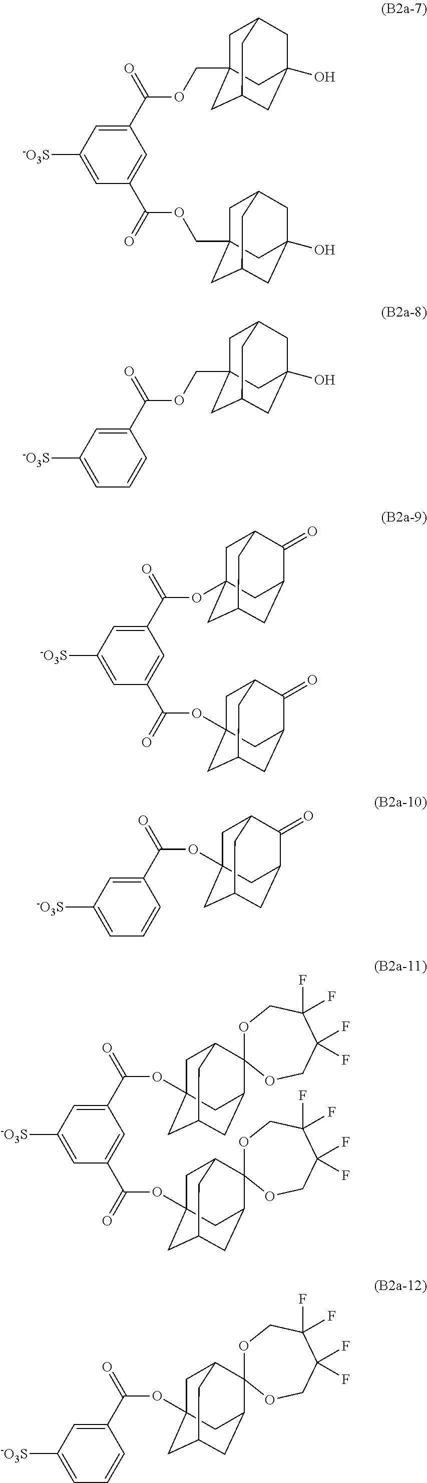

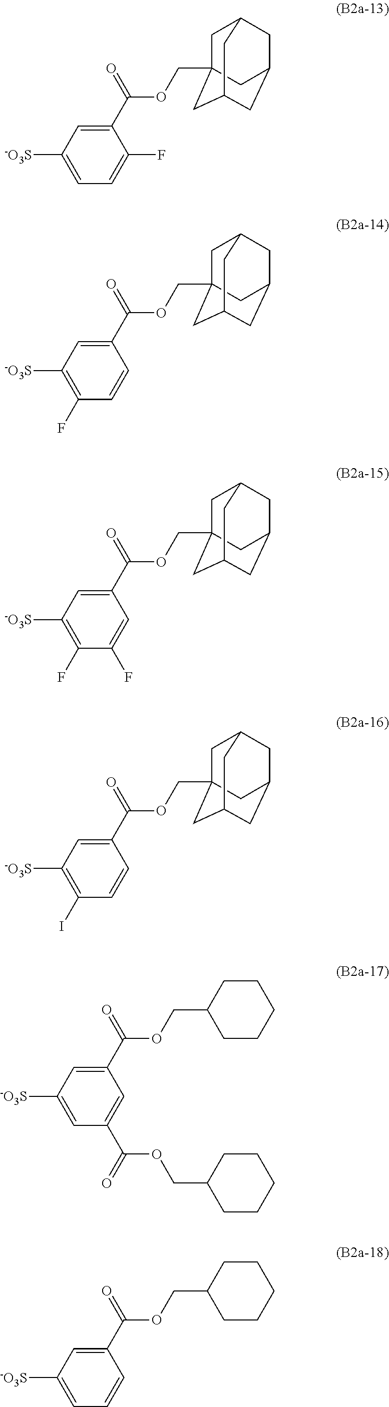

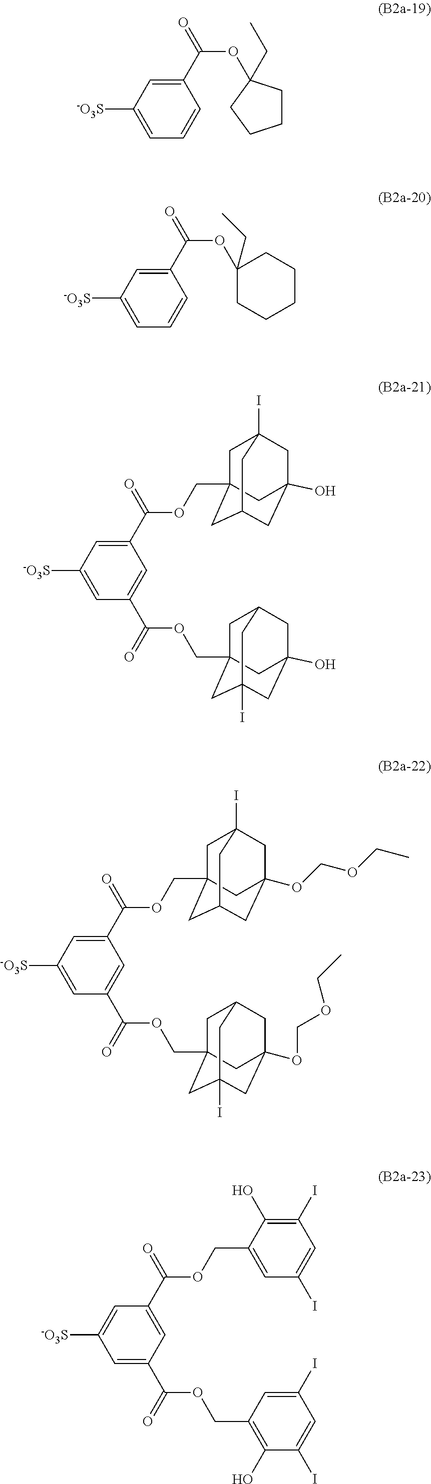

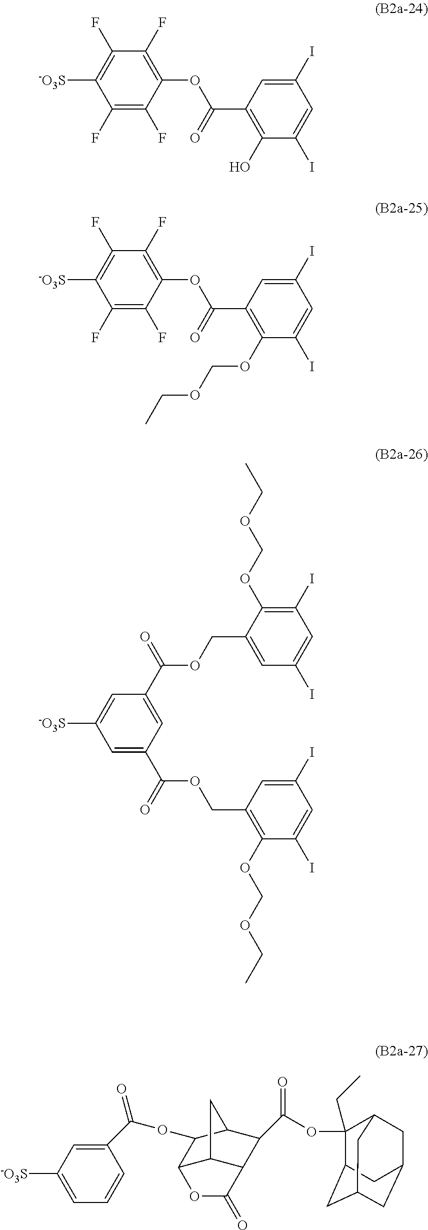

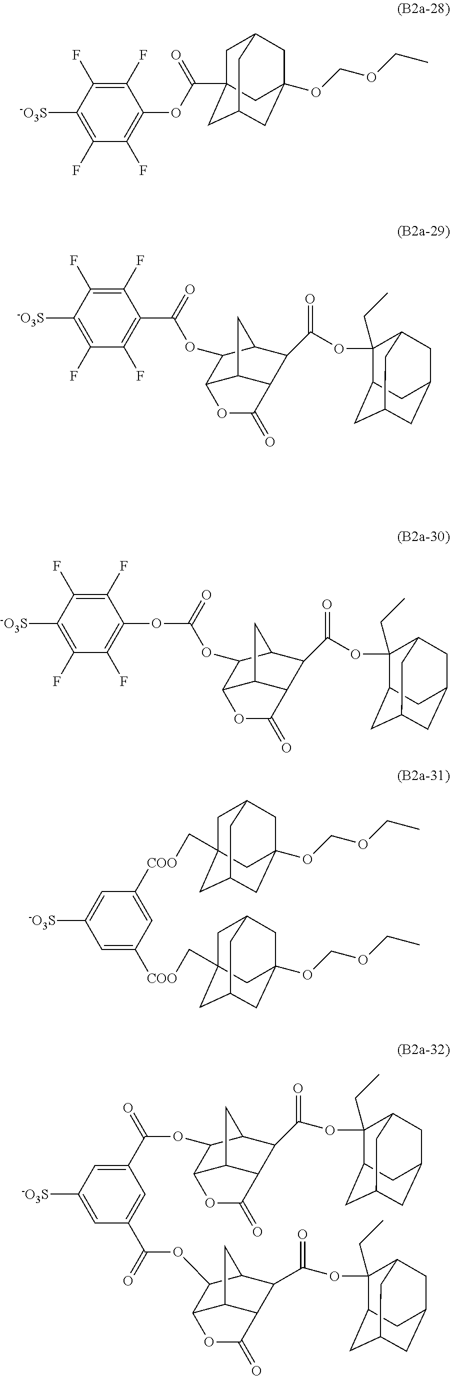

Examples of the acid generating agent (B2) include a salt represented by the following formula:

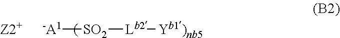

wherein
A¹ represents a nitrogen atom or a carbon atom;
L^{b2′} represents a single bond, or a divalent hydrocarbon group having 1 to 24 carbon atoms and optionally having a substituent, and —CH₂— contained in the hydrocarbon group is optionally replaced with —O—, —S—, —CO—, —SO— or —SO₂—;
Y^{b1′} represents a methyl group optionally having a substituent, or a cyclic hydrocarbon group having 3 to 24 carbon atoms and optionally having a substituent, and —CH₂— contained in the cyclic hydrocarbon group is optionally replaced with —O—, —S—, —CO—, —SO— or —SO₂—;
nb5 represents an integer of 2 or 3; when nb5 is 2, A¹ is a nitrogen atom, and when nb5 is 3, A¹ is a carbon atom; a plurality of groups in parentheses may be the same or different from each other, and two groups in parentheses may be bonded to form a ring containing A¹; and
Z2⁺ represents an organic cation.

In formula (B2), examples of L^{b2′} and Y^{b1′} include the same groups as L^{b2} and Y^{b1} in formula (B1-A1).

L^{b2′} is preferably a single bond, *-L^{b23}-, *-L^{b23}-X²— or *-L^{b23}-X²—W²—X³—, where L^{b23} represents a chain hydrocarbon group having 1 to 6 carbon atoms and optionally having a fluorine atom; X² and X³ each independently represent —O—, —CO—O—, —O—CO—, —O—CO—O— or —O—; W² represents an alicyclic hydrocarbon group having 3 to 12 carbon atoms, and —CH₂— contained in the alicyclic hydrocarbon group is optionally replaced with —O— or —CO—; and represents a bonding site to SO₂.

Y^{b1′} is preferably a methyl group having a fluorine atom, or a cyclic hydrocarbon group having 3 to 20 carbon atoms and optionally having a substituent, where —CH₂— contained in the cyclic hydrocarbon group is optionally replaced with —O—, —CO—, —S—, —SO— or —SO₂—, and is more preferably a trifluoromethyl group, an alicyclic hydrocarbon group having 3 to 18 carbon atoms and optionally having a substituent, where —CH₂— contained in the alicyclic hydrocarbon group is optionally replaced with —O—, —S—, —CO—, —SO— or —SO₂—, or an aromatic hydrocarbon group having 6 to 18 carbon atoms and optionally having a substituent. Specifically, it is preferably a trifluoromethyl group, or a group represented by any one of formulas (Y1) to (Y36) exemplified in formula (B1-A1).

In the anion of the salt represented by formula (B2), when two —SO₂-L^{b2′}-Y^{b1′} are taken together to form a ring containing A¹, examples thereof include an anion represented by formula (B2′):

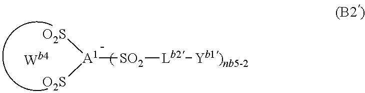

wherein
A¹, Y^{b1′}, L^{b2′} and nb5 have the same meanings as in formula (B2); and
W^{b4} represents a disulfonyl imide ring or a disulfonyl methide ring, each of which has 2 to 12 carbon atoms and optionally has a fluorine atom.

The disulfonyl imide ring or disulfonyl methide ring of W^{b4} preferably has 3 to 12 carbon atoms, more preferably has 3 to 6 carbon atoms, and it is preferable that a hydrogen atom of a methylene group contained in the ring is replaced with a fluorine atom.

Examples of the anion of the salt represented by formula (B2) include the following. Among the anions, the anions represented by formulas (B3a-1), (B3a-2), and (B3a-12) to (B3a-14) are preferable.

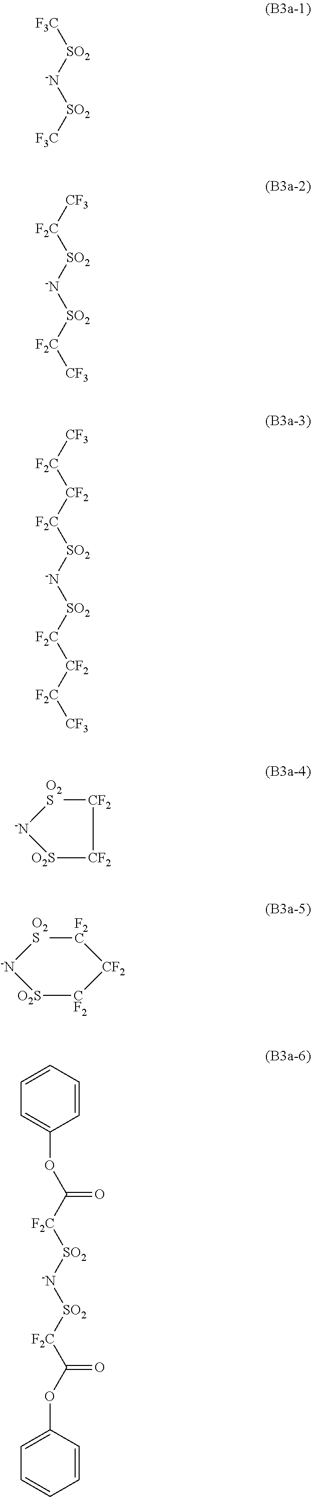

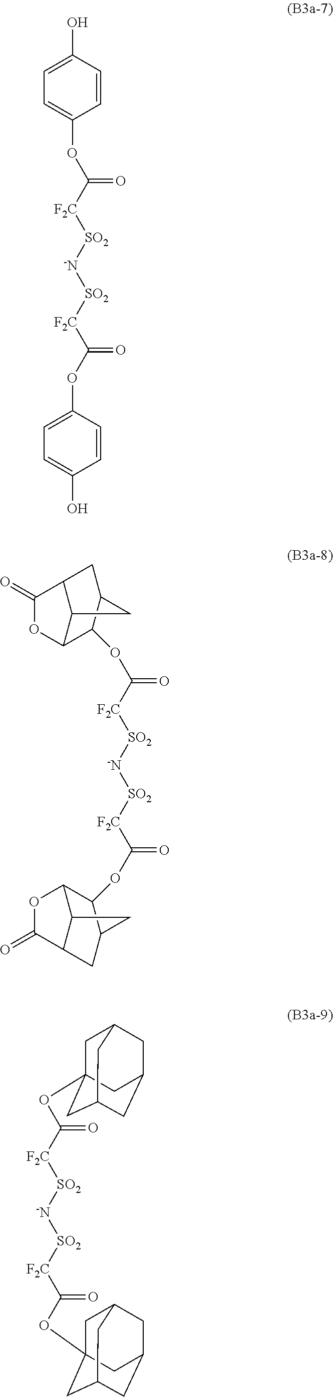

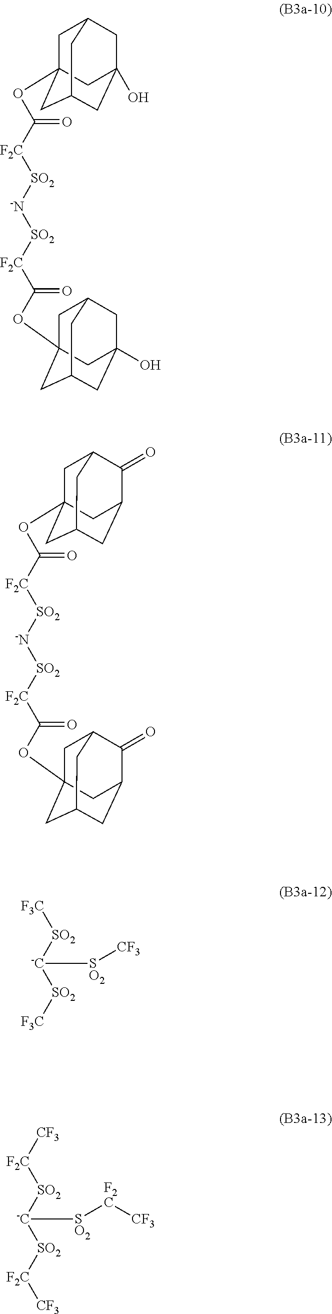

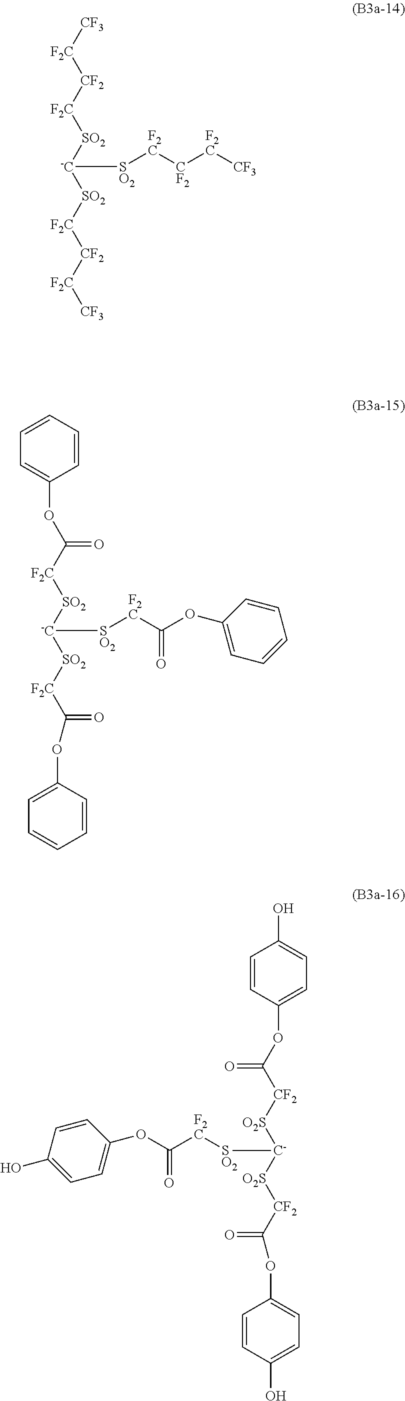

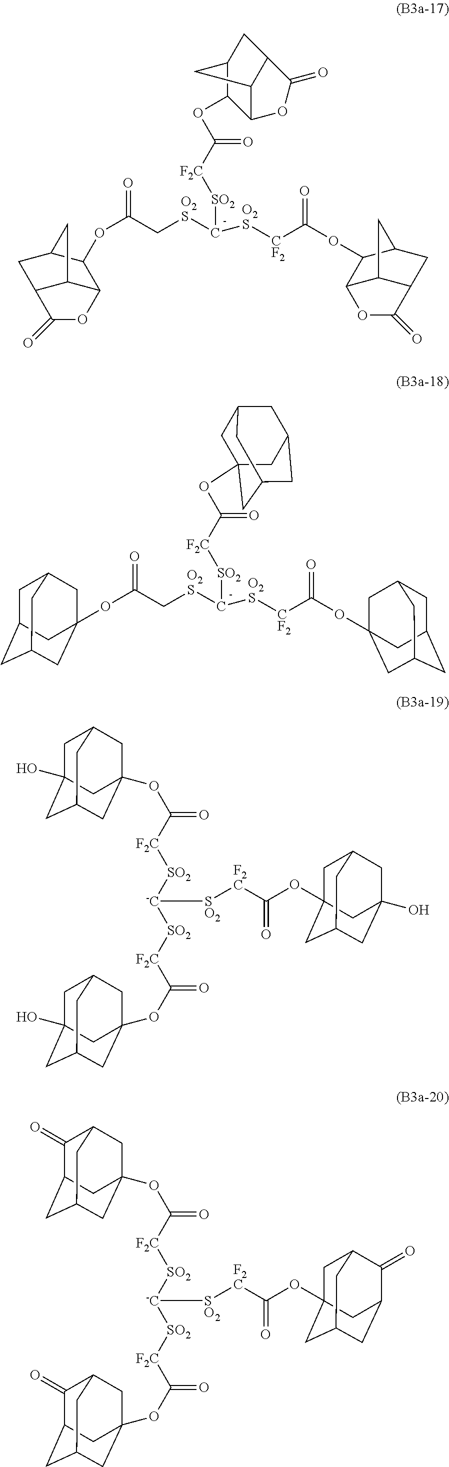

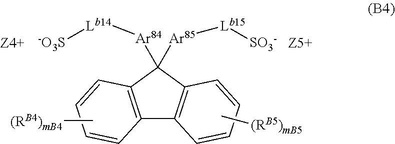

wherein
L^{b14} and L^{b15} each independently represent a single bond, or a divalent hydrocarbon group having 1 to 72 carbon atoms and optionally having a substituent, and —CH₂— contained in the hydrocarbon group is optionally replaced with —O—, —S—, —CO—, —SO— or —SO₂—;
Ar^{B4} and Ar^{B5} each independently represent an aromatic hydrocarbon group having 6 to 24 carbon atoms and optionally having a substituent, and substituents of Ar^{B4} and Ar^{B5} may be bonded to form a ring;
R^{B4} and R^{B5} each independently represent a halogen atom, or a hydrocarbon group having 1 to 18 carbon atoms and optionally having a substituent, and —CH₂— contained in the hydrocarbon group is optionally replaced with —O—, —CO—, —S—, —SO— or —SO₂—;
mB4 represents an integer of 0 to 4, and when mB4 is 2 or larger, a plurality of R^{B4} may be the same or different from each other;
mB5 represents an integer of 0 to 4, and when mB5 is 2 or larger, a plurality of R^{B5} may be the same or different from each other; and
Z4⁺ and Z5⁺ each independently represent an organic cation.

In formula (B4), examples of L^{b14} and L^{b15} include the same groups as L^{b1} in formula (B1) or L^{b2}, L^{b3} and the like in formula (B1-A1).

Examples of the aromatic hydrocarbon group of Ar^{B4} and Ar^{B5} include a phenylene group, a naphthylene group, an anthrylene group, a biphenylene group and a phenanthrylene group. The bonding site can be set to any position. The number of carbon atoms of the aromatic hydrocarbon group is preferably 6 to 18, is more preferably 6 to 14, and is further preferably 6 to 10.

Examples of the substituent that the aromatic hydrocarbon group of Ar^{B4} and Ar^{B5} optionally has include a halogen atom, a hydroxy group, a cyano group, a carboxy group, an alkyl group having 1 to 12 carbon atoms, a fluorinated alkyl group having 1 to 6 carbon atoms, an alkoxy group having 1 to 12 carbon atoms, and a combined group of two or more of these groups. Examples of these substituents include the same as those exemplified for R^{B4} and R^{B5}, which will be described later.

In particular, Ar^{B4} and Ar^{B5} are preferably aromatic hydrocarbon groups having 6 to 10 carbon atoms and optionally having a substituent, are more preferably aromatic hydrocarbon groups having 6 to 10 carbon atoms and optionally having a halogen atom, a hydroxy group, an alkyl group having 1 to 12 carbon atoms or a fluorinated alkyl group having 1 to 6 carbon atoms, are further preferably aromatic hydrocarbon groups having 6 to 10 carbon atoms and optionally having a fluorine atom, an iodine atom, an alkyl group having 1 to 6 carbon atoms or a fluorinated alkyl group having 1 to 3 carbon atoms, and are further more preferably phenylene groups or naphthylene groups.

When Ar^{B4} and Ar^{B5} are phenylene groups optionally having a substituent, the bonding position of the fluorene ring may be any of the ortho, meta and para positions with respect to the bonding positions of L^{b24} and L^{b25}, but it is preferably at the meta position or the para position, and is more preferably at the para position.

Examples of the halogen atom of R^{B4} and R^{B5} include a fluorine atom, a chlorine atom, a bromine atom and an iodine atom.

Examples of the hydrocarbon group having 1 to 18 carbon atoms of R^{B4} and R^{B5} include a chain hydrocarbon group such as an alkyl group and an alkanediyl group, an alicyclic hydrocarbon group, an aromatic hydrocarbon group, and a group formed by combining these groups.

The alkyl group is a straight-chain or branched alkyl group, and examples thereof include a methyl group, an ethyl group, a n-propyl group, an isopropyl group, a n-butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group.

The alkanediyl group is a straight-chain or branched alkanediyl group, and examples thereof include: a straight-chain alkanediyl group such as a methylene group, an ethylene group, a propane-1,3-diyl group, a butane-1,4-diyl group, a pentane-1,5-diyl group and a hexane-1,6-diyl group; and

a branched alkanediyl group such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group.

The number of carbon atoms of the chain hydrocarbon group is preferably 1 to 12, is more preferably 1 to 9, is further preferably 1 to 6, is still more preferably 1 to 4, and is still further preferably 1 to 3.

The alicyclic hydrocarbon group may be either monocyclic or polycyclic, and examples thereof include the groups represented as follows. The bonding site can be set to any position.

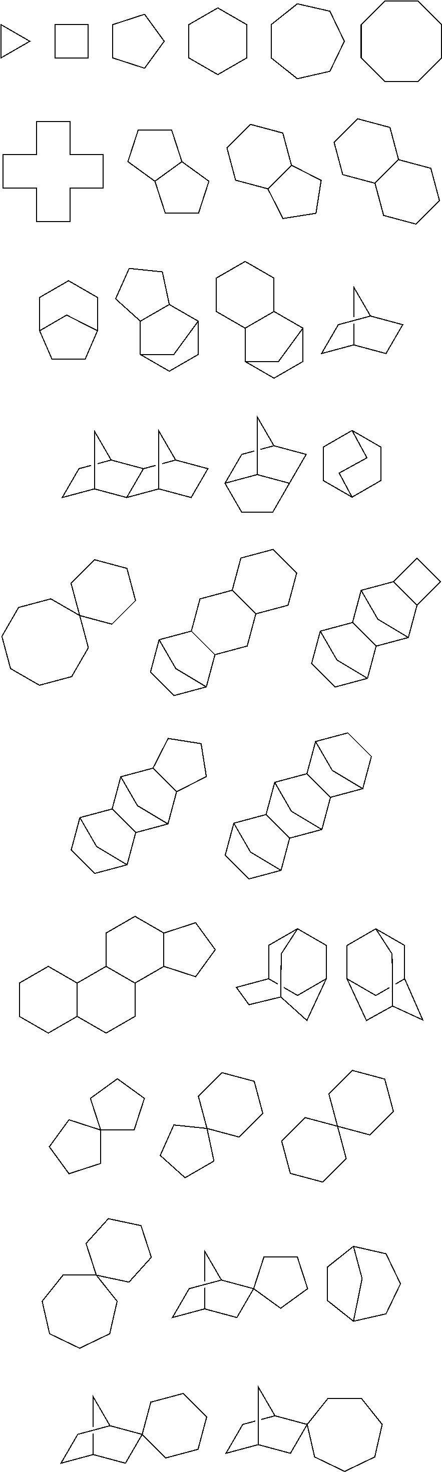

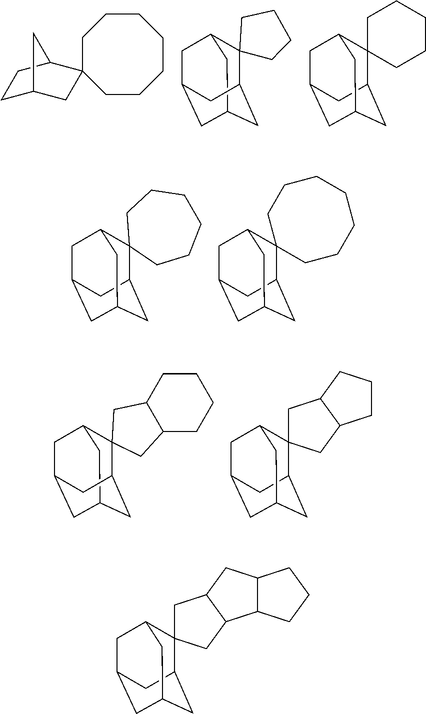

Specific examples of the monocyclic alicyclic hydrocarbon group include a monocyclic cycloalkyl group such as a cyclopropyl group, a cyclobutyl group, a cyclopentyl group, a cyclohexyl group, a cycloheptyl group, a cyclooctyl group, a cyclononyl group, a cyclodecyl group and a cyclododecyl group. Examples of the polycyclic alicyclic hydrocarbon group include a polycyclic cycloalkyl group such as a decahydronaphthyl group, an adamantyl group and a norbornyl group, and a Spiro ring having a cycloalkyl group bonded by Spiro to each of a cycloalkyl group, a norbornyl group or an adamantyl group, such as a spirocyclohexane-1,2′-cyclopentane group and a spiroadamantane-2,3′-cyclopentane group. The number of carbon atoms of the alicyclic hydrocarbon group is preferably 3 to 18, is more preferably 3 to 16, and is further preferably 3 to 12.

Examples of the aromatic hydrocarbon group include a phenyl group, a naphthyl group, a biphenyl group, an anthryl group, a phenanthryl group and a binaphthyl group. The number of carbon atoms of the aromatic hydrocarbon group is preferably 6 to 18, is more preferably 6 to 14, and is further preferably 6 to 10.

Examples of the group formed by combining two or more groups among a chain hydrocarbon group, an alicyclic hydrocarbon group and an aromatic hydrocarbon group include a combined group of an aromatic hydrocarbon group and a chain hydrocarbon group (such as an aromatic hydrocarbon group-alkanediyl group-*, an alkyl group-aromatic hydrocarbon group-*, and an alkyl group-aromatic hydrocarbon group-alkanediyl group-*, where —CH₂— contained in the alkanediyl group and the alkyl group is optionally replaced with —O—, —CO—, —S—, —SO— or —SO₂—), a combined group of an alicyclic hydrocarbon group and a chain hydrocarbon group (such as an alicyclic hydrocarbon group-alkanediyl group-*, an alkyl group-alicyclic hydrocarbon group-*, and an alkyl group-alicyclic hydrocarbon group-alkanediyl group-*, where —CH₂— contained in the alicyclic hydrocarbon group, the alkanediyl group and the alkyl group is optionally replaced with —O—, —CO—, —S—, —SO— or —SO₂—), and a combined group of an aromatic hydrocarbon group and an alicyclic hydrocarbon group (such as an aromatic hydrocarbon group-alicyclic hydrocarbon group-* and an alicyclic hydrocarbon group-aromatic hydrocarbon group-*, where —CH₂— contained in the alicyclic hydrocarbon group is optionally replaced with —O—, —CO—, —S—, —SO— or —SO₂—):* represents a bonding site.

Examples of the aromatic hydrocarbon group-alkanediyl group-* include an aralkyl group such as a benzyl group and a phenethyl group.

Examples of the alkyl group-aromatic hydrocarbon group-* include a tolyl group, a xylyl group, and a cumenyl group.

Examples of the alicyclic hydrocarbon group-alkanediyl group-* include a cycloalkylalkyl group such as a cyclohexylmethyl group, a cyclohexylethyl group, a 1-(adamantan-1-yl)methyl group and a 1-(adamantan-1-yl)-1-methylethyl group.

Examples of the alkyl group-alicyclic hydrocarbon group-* include a cycloalkyl group having an alkyl group such as a methylcyclohexyl group, a dim ethylcyclohexyl group and a 2-alkyladamantan-2-yl group.

Examples of the aromatic hydrocarbon group-alicyclic hydrocarbon group-* include a phenylcyclohexyl group.

Examples of the alicyclic hydrocarbon group-aromatic hydrocarbon group-* include a cyclohexylphenyl group.

In the combination, two or more types of alicyclic hydrocarbon groups, aromatic hydrocarbon groups, and chain hydrocarbon groups may each be combined. In addition, any of the groups may be bonded to a benzene ring or the like.

When —CH₂— contained in the hydrocarbon group represented by R^{B4} and R^{B5} is replaced with —O—, —CO—, —S—, —SO— or —SO₂—, the number of carbon atoms before replacement corresponds to the defined total number of carbon atoms of the hydrocarbon group. In addition, the number thereof may be one, or may be two or more.

Examples of the group formed by replacing —CH₂— contained in the hydrocarbon group with —O—, —S—, —SO₂—, —SO— or —CO— include, as described above, a hydroxy group, a thiol group, a carboxy group, an alkoxy group, an alkoxycarbonyl group, an alkylcarbonyl group, an alkylcarbonyloxy group, an alkylthio group, an alkylsulfonyl group, an oxy group, a carbonyl group, a thio group, a sulfonyl group, an alkanediyloxy group, an alkanediyloxycarbonyl group, an alkanediylcarbonyl group, an alkanediylcarbonyloxy group, an alkanediylthio group, an alkanediylsulfonyl group, a cycloalkoxy group, a cycloalkylalkoxy group, an alkoxycarbonyloxy group, an aromatic hydrocarbon group-carbonyloxy group, an aromatic hydrocarbon group-carbonyl group, an aromatic hydrocarbon group-oxy group and a combined group of two or more of these groups. Examples of these groups formed by the replacement include the same groups as those exemplified in the present specification, within the range permitted by the upper limit of the number of carbon atoms.

In addition, examples of the group formed by replacing —CH₂-contained in the alicyclic hydrocarbon group and the combined group of an alicyclic hydrocarbon group and an aromatic hydrocarbon group with —O—, —S—, —SO₂—, —SO— or —CO— include the same groups as those exemplified in the present specification, within the range permitted by the upper limit of the number of carbon atoms.

Examples of the substituent that the hydrocarbon group of R^{B4} and R^{B5} optionally has include a halogen atom, a cyano group and a nitro group.

Examples of the halogen atom include the same groups as described above.

The hydrocarbon group optionally has one substituent or a plurality of substituents.

When R^{B4} and R^{B5} are hydrocarbon groups having a halogen atom, examples of the hydrocarbon group having a halogen atom of R^{B4} and R^{B5} include a chain hydrocarbon group having a halogen atom such as a haloalkyl group, an alicyclic hydrocarbon group having a halogen atom, and an aromatic hydrocarbon group having a halogen atom.

The haloalkyl group represents an alkyl group having a halogen atom, and examples thereof include a haloalkyl group having 1 to 12 carbon atoms such as a fluorinated alkyl group having 1 to 12 carbon atoms, a chlorinated alkyl group having 1 to 12 carbon atoms, a brominated alkyl group having 1 to 12 carbon atoms, and an iodinated alkyl group having 1 to 12 carbon atoms. Examples of the haloalkyl group include a perfluoroalkyl group having 1 to 12 carbon atoms (such as a trifluoromethyl group, a pentafluoroethyl group, a heptafluoropropyl group and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group and an iodomethyl group. The number of carbon atoms of the haloalkyl group is preferably 1 to 9, is more preferably 1 to 6, is further preferably 1 to 4, and is further more preferably 1 to 3.

The —CH₂— contained in the haloalkyl group is optionally replaced with —O—, —CO— or the like, and examples of such a group include a haloalkoxy group, a haloalkoxycarbonyl group, a haloalkylcarbonyl group and a haloalkylcarbonyloxy group. Specific examples thereof include a haloalkoxy group having 1 to 11 carbon atoms, a haloalkoxycarbonyl group having 2 to 11 carbon atoms, a haloalkylcarbonyl group having 2 to 12 carbon atoms and a haloalkylcarbonyloxy group having 2 to 11 carbon atoms, such as a group in which one or more hydrogen atoms of the groups exemplified above are replaced with halogen atoms.

mB4 is preferably 0, 1, 2 or 3, is more preferably 0, 1 or 2, and is further preferably 0 or 1.

mB5 is preferably 0, 1, 2 or 3, is more preferably 0, 1 or 2, and is further preferably 0 or 1.

R^{B4} and R^{B5} are each independently preferably a halogen atom, a haloalkyl group having 1 to 6 carbon atoms or an alkyl group having 1 to 6 carbon atoms, where —CH₂— contained in the haloalkyl group and the alkyl group is optionally replaced with —O— or —CO—, more preferably a fluorine atom, an iodine atom, a haloalkyl group having 1 to 6 carbon atoms or an alkyl group having 1 to 6 carbon atoms, where —CH₂— contained in the haloalkyl group and the alkyl group is optionally replaced with —O— or —CO—, further preferably a fluorine atom, an iodine atom, a haloalkyl group having 1 to 4 carbon atoms or an alkyl group having 1 to 4 carbon atoms, where —CH₂— contained in the alkyl group is optionally replaced with —O— or —CO—, still more preferably a fluorine atom, an iodine atom, a hydroxy group, an alkyl group having 1 to 4 carbon atoms, a haloalkyl group having 1 to 4 carbon atoms or an alkoxy group having 1 to 3 carbon atoms, still further preferably a fluorine atom, an iodine atom, a hydroxy group, an alkyl group having 1 to 4 carbon atoms, a perfluoroalkyl group having 1 to 4 carbon atoms or an alkoxy group having 1 to 3 carbon atoms, and further more preferably a fluorine atom, an iodine atom, a trifluoromethyl group, a methyl group, a methoxy group, an ethoxy group or a hydroxy group.

Examples of the anion in the salt represented by formula (B4) include the following. Among the anions, the anions represented by formulas (B4a-1) to (B4a-4) and (B4a-11) to (B4a-14) are preferable.

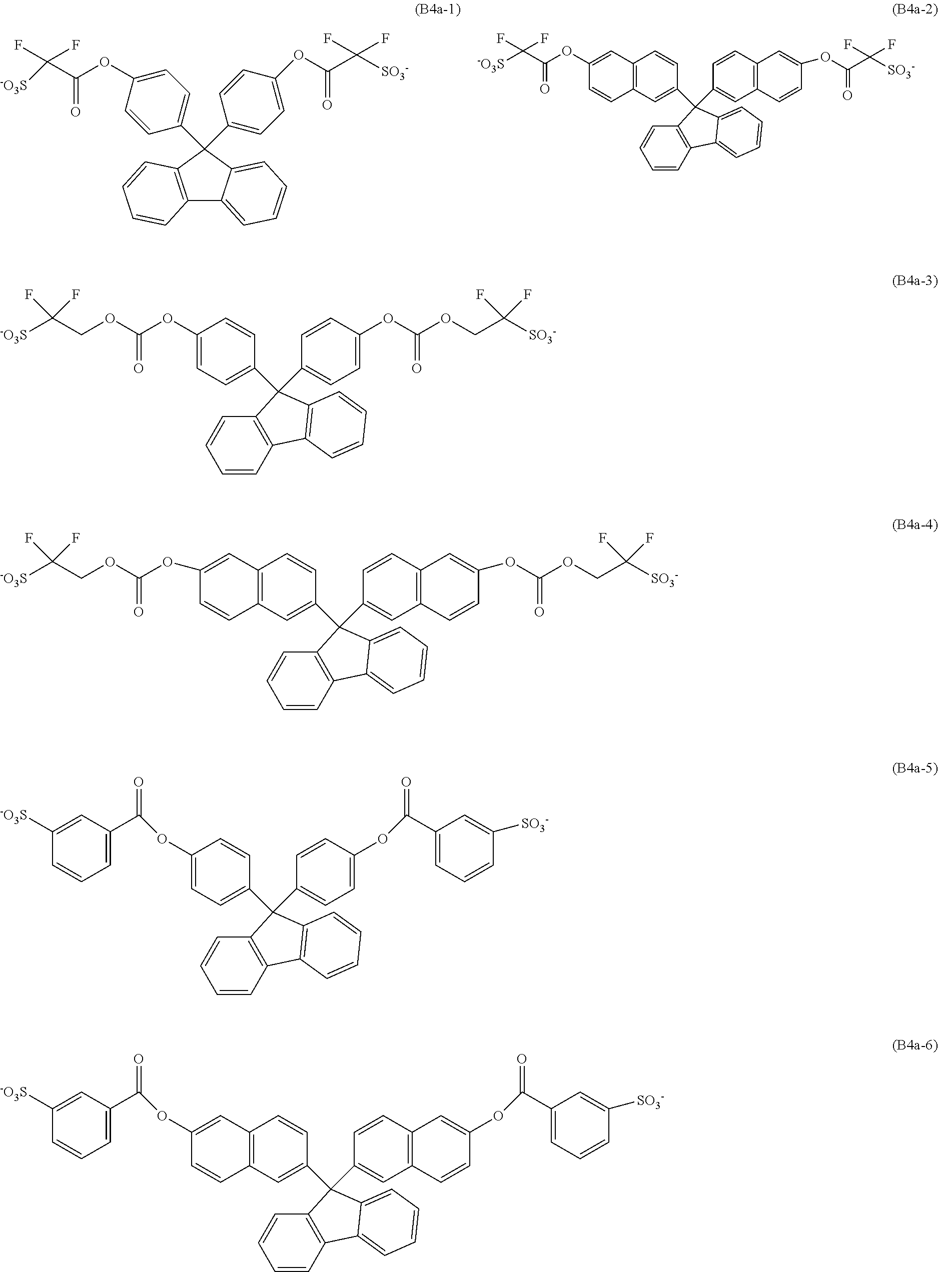

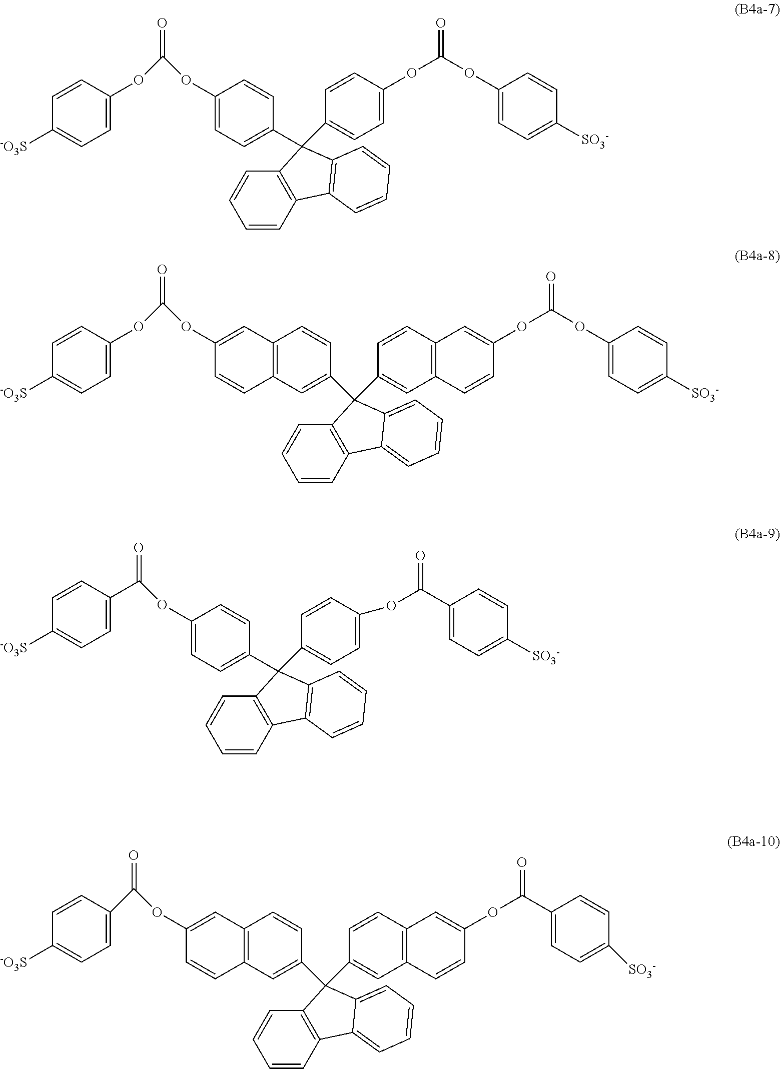

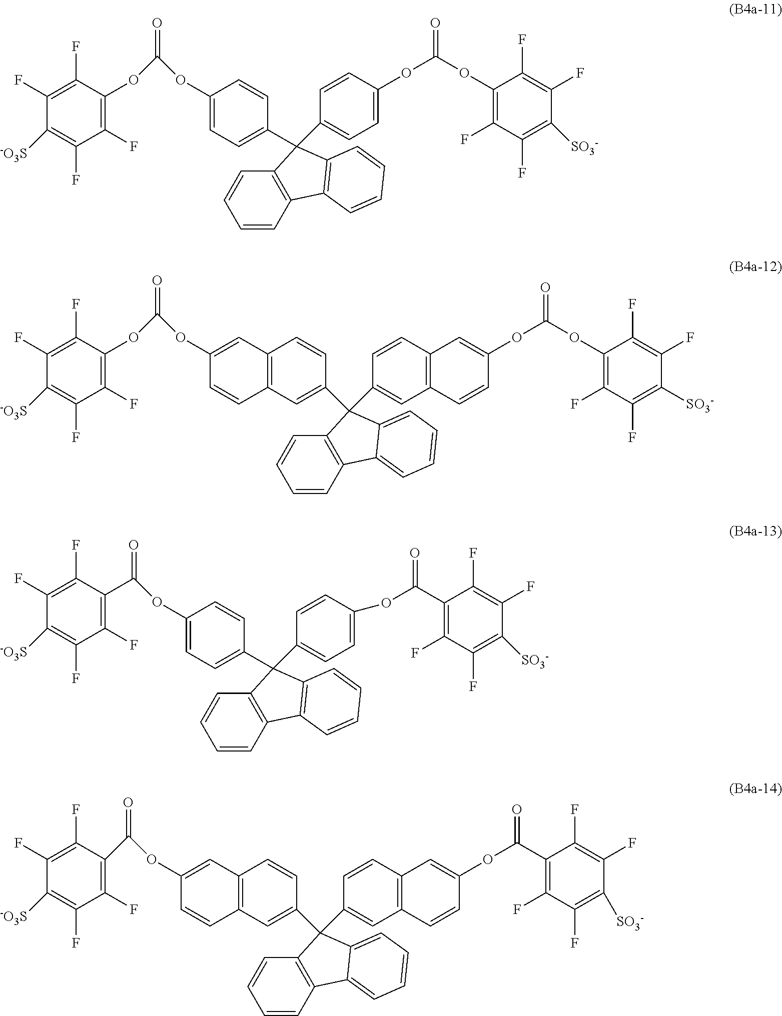

In addition, in another embodiment of the compound (B), a salt obtained by replacing the anion in the salt represented by formula (B1) with a carboxylate anion is also suitably used as the compound (B).

Examples of the carboxylate anion include the following anions.

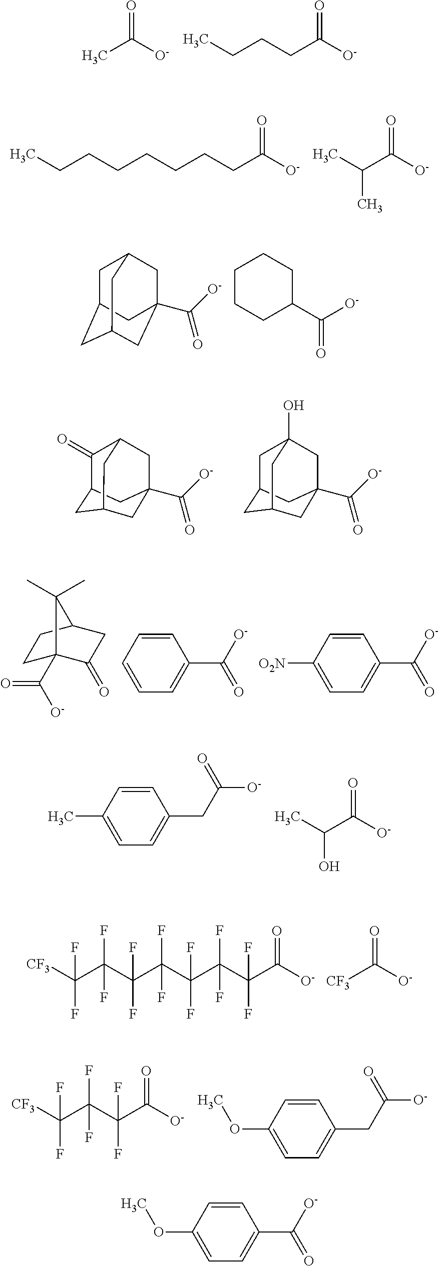

Examples of the organic cation of Z1⁺, Z2⁺, Z4⁺ and Z5⁺ include an organic onium cation, an organic sulfonium cation, an organic iodonium cation, an organic ammonium cation, a benzothiazolium cation and an organic phosphonium cation. Among these cations, an organic sulfonium cation and an organic iodonium cation are preferable, and an arylsulfonium cation and an aryliodonium cation are more preferable. Specific examples thereof include a cation represented by any one of formulas (b2-1) to (b2-5) (hereinafter referred to as “cation (b2-1)” or the like according to the formula number in some cases):

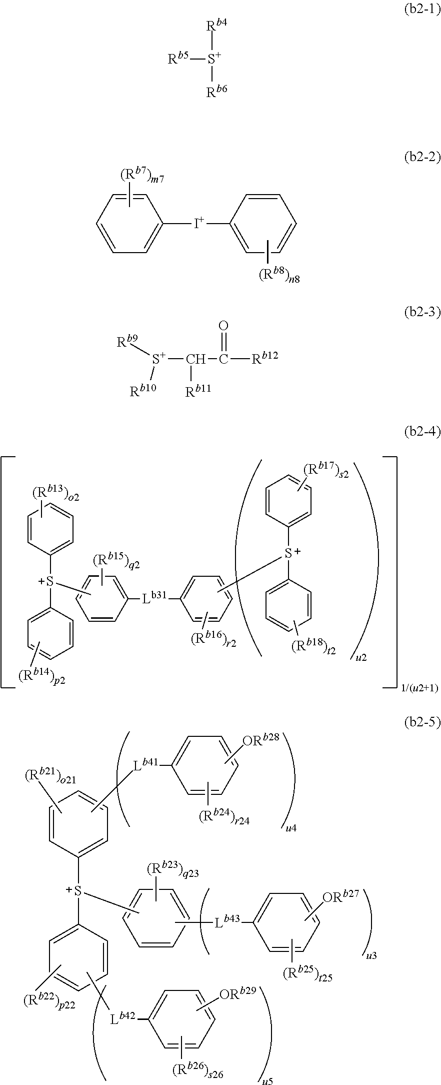

wherein
R^{b4} to R^{b6} each independently represent a chain hydrocarbon group having 1 to 30 carbon atoms, an alicyclic hydrocarbon group having 3 to 36 carbon atoms or an aromatic hydrocarbon group having 6 to 36 carbon atoms, where a hydrogen atom contained in the chain hydrocarbon group is optionally replaced with a hydroxy group, an alkoxy group having 1 to 12 carbon atoms, an alicyclic hydrocarbon group having 3 to 12 carbon atoms or an aromatic hydrocarbon group having 6 to 18 carbon atoms, a hydrogen atom contained in the alicyclic hydrocarbon group is optionally replaced with a halogen atom, an aliphatic hydrocarbon group having 1 to 18 carbon atoms, an alkylcarbonyl group having 2 to 4 carbon atoms or a glycidyloxy group, and a hydrogen atom contained in the aromatic hydrocarbon group is optionally replaced with a halogen atom, a hydroxy group, an aliphatic hydrocarbon group having 1 to 18 carbon atoms, a fluorinated alkyl group having 1 to 12 carbon atoms or an alkoxy group having 1 to 12 carbon atoms;
R^{b4} and R^{b5} may be bonded to each other to form a ring together with a sulfur atom to which they are bonded, and —CH₂— contained in the ring is optionally replaced with —O—, —S— or —CO—;
R^{b7} and R^{b8} each independently represent a halogen atom, a hydroxy group, an aliphatic hydrocarbon group having 1 to 12 carbon atoms, a fluorinated alkyl group having 1 to 12 carbon atoms or an alkoxy group having 1 to 12 carbon atoms;
m7 and n8 each independently represent an integer of 0 to 5;
when m7 is 2 or larger, a plurality of R^{b7} may be the same or different from each other, and when n8 is 2 or larger, a plurality of R^{b8} may be the same or different from each other;
R^{b9} and R^{b10} each independently represent a chain hydrocarbon group having 1 to 36 carbon atoms or an alicyclic hydrocarbon group having 3 to 36 carbon atoms;
R^{b9} and R^{b10} may be bonded to each other to form a ring together with a sulfur atom to which they are bonded, and —CH₂— contained in the ring is optionally replaced with —O—, —S— or —CO—;
R^{b1l} represents a hydrogen atom, a chain hydrocarbon group having 1 to 36 carbon atoms, an alicyclic hydrocarbon group having 3 to 36 carbon atoms or an aromatic hydrocarbon group having 6 to 18 carbon atoms;
R^{b12} represents a chain hydrocarbon group having 1 to 12 carbon atoms, an alicyclic hydrocarbon group having 3 to 18 carbon atoms or an aromatic hydrocarbon group having 6 to 18 carbon atoms, where a hydrogen atom contained in the chain hydrocarbon group is optionally replaced with an aromatic hydrocarbon group having 6 to 18 carbon atoms, and a hydrogen atom contained in the aromatic hydrocarbon group is optionally replaced with an alkoxy group having 1 to 12 carbon atoms or an alkylcarbonyloxy group having 1 to 12 carbon atoms;
R^{b11} and R^{b12} may be bonded to each other to form a ring containing —CH—CO— to which they are bonded, and —CH₂— contained in the ring is optionally replaced with —O—, —S— or —CO—;
R^{b13} to R^{b18} and R^{b21} to R^{b26} each independently represent a halogen atom, a hydroxy group, an aliphatic hydrocarbon group having 1 to 12 carbon atoms, a fluorinated alkyl group having 1 to 12 carbon atoms or an alkoxy group having 1 to 12 carbon atoms;
R^{b13} and R^{b14} and R^{b21} and R^{b22} may be bonded to each other to form a sulfur atom-containing ring together with a benzene ring to which they are bonded, and —CH₂— contained in the ring is optionally replaced with —O—, —S— or —CO—;
R^{b27} to R^{b29} each independently represent a hydrogen atom or a group containing an acid labile group;
L^{b31} and L^{b4l} to L^{b43} each independently represent a sulfur atom or an oxygen atom;
o2, p2, s2 and t2 each independently represent an integer of 0 to 5;
q2 and r2 each independently represent an integer of 0 to 4;
u2 represents 0 or 1;
when o2 is 2 or larger, a plurality of R^{b13} are the same or different from each other, when p2 is 2 or larger, a plurality of R^{b14} are the same or different from each other, when q2 is 2 or larger, a plurality of R^{b15} are the same or different from each other, when r2 is 2 or larger, a plurality of R^{b16} are the same or different from each other, when s2 is 2 or larger, a plurality of R^{b17} are the same or different from each other, and when t2 is 2 or larger, a plurality of R^{b18} are the same or different from each other;
u3 represents an integer of 1 to 3, and 1≤u3+q23≤5 is satisfied;
u4 and u5 each independently represent an integer of 0 to 3, and 0≤u4+o21≤5 and 0≤u5+p22≤5 are satisfied;
when u3, u4 and u5 are each 2 or larger, a plurality of groups in parentheses are the same or different from each other;
o21, p22, q23, r24, t25 and s26 each independently represent an integer of 0 to 4; and
when o21, p22, q23, r24, t25 and s26 are each 2 or larger, a plurality of R^{b21} to R^{b26} are the same or different from each other.

When u2 is 0, it is preferable that any one of o2, p2, q2 and r2 is 1 or larger, and at least one of R^{b13} to R^{b16} is a halogen atom, and when u2 is 1, it is preferable that any one of o2, p2, s2, t2, q2 and r2 is 1 or larger, and at least one of R^{b13} to R^{b18} is a halogen atom.

Furthermore, when u2 is 0, r2 is preferably 1 or larger, and is further preferably 1. In addition, when u2 is 0 and r2 is 1 or larger, R^{b16} is preferably a halogen atom.

The aliphatic hydrocarbon group represents a chain hydrocarbon group and an alicyclic hydrocarbon group.

Examples of the chain hydrocarbon group include an alkyl group such as a methyl group, an ethyl group, a propyl group, an isopropyl group, a butyl group, a sec-butyl group, a tert-butyl group, a pentyl group, a hexyl group, an octyl group and a 2-ethylhexyl group.

In particular, the chain hydrocarbon group of R^{b9} to R^{b12} preferably has 1 to 12 carbon atoms.

The alicyclic hydrocarbon group may be either monocyclic or polycyclic, and examples of the monocyclic alicyclic hydrocarbon group include a cycloalkyl group such as a cyclopropyl group, a cyclobutyl group, a cyclopentyl group, a cyclohexyl group, a cycloheptyl group, a cyclooctyl group and a cyclodecyl group. Examples of the polycyclic alicyclic hydrocarbon group include a decahydronaphthyl group, an adamantyl group, a norbornyl group and the following groups.

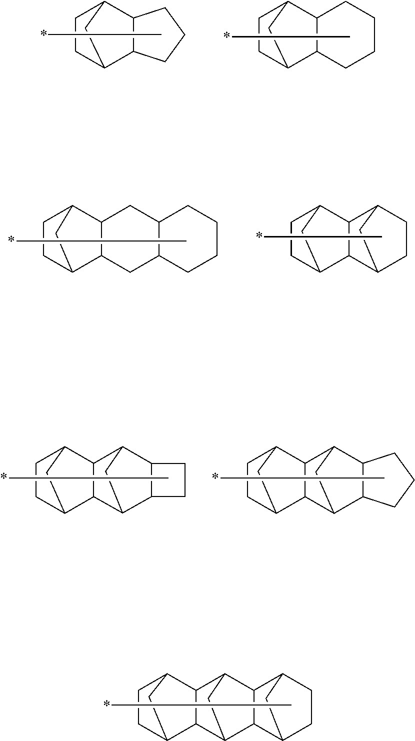

In particular, the alicyclic hydrocarbon group of R^{b9} to R^{b12} preferably has 3 to 18 carbon atoms, and more preferably has 4 to 12 carbon atoms.

Examples of the alicyclic hydrocarbon group in which a hydrogen atom is replaced with an aliphatic hydrocarbon group include a methylcyclohexyl group, a dim ethylcyclohexyl group, a 2-methyladamantane-2-yl group, a 2-ethyladamantane-2-yl group, a 2-isopropyladamantane-2-yl group, a methylnorbornyl group and an isobornyl group. In the alicyclic hydrocarbon group in which a hydrogen atom is replaced with an aliphatic hydrocarbon group, the total number of carbon atoms of the alicyclic hydrocarbon group and the aliphatic hydrocarbon group is preferably 20 or less.

The fluorinated alkyl group represents an alkyl group having 1 to 12 carbon atoms and a fluorine atom, and examples thereof include a fluoromethyl group, a difluoromethyl group, a trifluoromethyl group and a perfluorobutyl group. The number of carbon atoms of the fluorinated alkyl group is preferably 1 to 9, is more preferably 1 to 6, and is further preferably 1 to 4.

Examples of the aromatic hydrocarbon group include an aryl group such as a phenyl group, a biphenyl group, a naphthyl group and a phenanthryl group. The aromatic hydrocarbon group optionally has a chain hydrocarbon group or an alicyclic hydrocarbon group, and examples thereof include an aromatic hydrocarbon group having a chain hydrocarbon group (a tolyl group, a xylyl group, a cumenyl group, a mesityl group, a p-ethylphenyl group, a p-tert-butylphenyl group, a 2,6-diethylphenyl group, a 2-methyl-6-ethylphenyl group, and the like), and an aromatic hydrocarbon group having an alicyclic hydrocarbon group (a p-cyclohexylphenyl group, a p-adamantylphenyl group, and the like).

For information, when the aromatic hydrocarbon group has a chain hydrocarbon group or an alicyclic hydrocarbon group, a chain hydrocarbon group having 1 to 18 carbon atoms and an alicyclic hydrocarbon group having 3 to 18 carbon atoms are preferable.

Examples of the aromatic hydrocarbon group in which a hydrogen atom is replaced with an alkoxy group include a p-methoxyphenyl group.

Examples of the chain hydrocarbon group in which a hydrogen atom is replaced with an aromatic hydrocarbon group include an aralkyl group such as a benzyl group, a phenethyl group, a phenylpropyl group, a trityl group, a naphthylmethyl group and a naphthylethyl group.

Examples of the alkoxy group include a methoxy group, an ethoxy group, a propoxy group, a butoxy group, a pentyloxy group, a hexyloxy group, a heptyloxy group, an octyloxy group, a decyloxy group and a dodecyloxy group.

Examples of the alkylcarbonyl group include an acetyl group, a propionyl group and a butyryl group.

Examples of the halogen atom include a fluorine atom, a chlorine atom, a bromine atom and an iodine atom.

Examples of the alkylcarbonyloxy group include a methylcarbonyloxy group, an ethylcarbonyloxy group, a propylcarbonyloxy group, an isopropylcarbonyloxy group, a butylcarbonyloxy group, a sec-butylcarbonyloxy group, a tert-butylcarbonyloxy group, a pentylcarbonyloxy group, a hexylcarbonyloxy group, an octylcarbonyloxy group and a 2-ethylhexylcarbonyloxy group.

The ring formed by R^{b4} and R^{b5} that are bonded to each other together with a sulfur atom to which they are bonded may be any ring of a monocyclic type, a polycyclic type, an aromatic type, a non-aromatic type, a saturated type and an unsaturated type. Examples of the ring include a ring having 3 to 18 carbon atoms, and a ring having 4 to 18 carbon atoms is preferable. Examples of the sulfur atom-containing ring include a 3-membered ring to a 12-membered ring, and it is preferably a 3-membered ring to a 7-membered ring, and examples thereof include the following rings. * represents a bonding site.

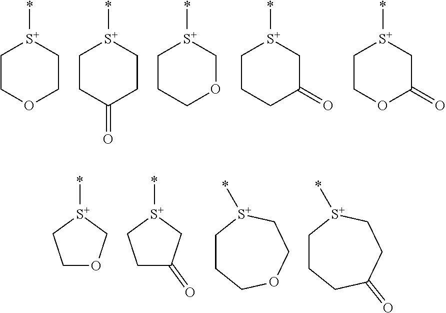

The ring formed by R^{b9} and R^{b10} together may be any ring of a monocyclic type, a polycyclic type, an aromatic type, a non-aromatic type, a saturated type and an unsaturated type. Examples of the ring include a 3 membered ring to a 12-membered ring, and it is preferably a 3-membered ring to a 7-membered ring. Examples thereof include a thiolan-1-ium ring (tetrahydrothiophenium ring), a thian-1-ium ring and a 1,4-oxathian-4-ium ring.

The ring formed by R^{b11} and R^{b12} together may be any ring of a monocyclic type, a polycyclic type, an aromatic type, a non-aromatic type, a saturated type and an unsaturated type. Examples of the ring include a 3 membered ring to a 12-membered ring, and it is preferably a 3-membered ring to a 7-membered ring. Examples thereof include an oxocycloheptane ring, an oxocyclohexane ring, an oxonorbornane ring and an oxoadamantane ring.

Examples of the group containing an acid labile group of R^{b27} to R^{b29} include a group represented by —R^{c30}, —CO—O—R^{c30} or -L^{c10}-CO—O—R^{c30} where L^{c10} represents an alkanediyl group having 1 to 6 carbon atoms and R^{c30} represents an acid labile group.

Examples of the acid labile group of R^{c30} include a group represented by formula (1) or (2).

Among the cations (b2-1) to (b2-5), the cation (b2-1), the cation (b2-4) or the cation (b2-5) is preferable.

Examples of the cation (b2-1) include the following cations.

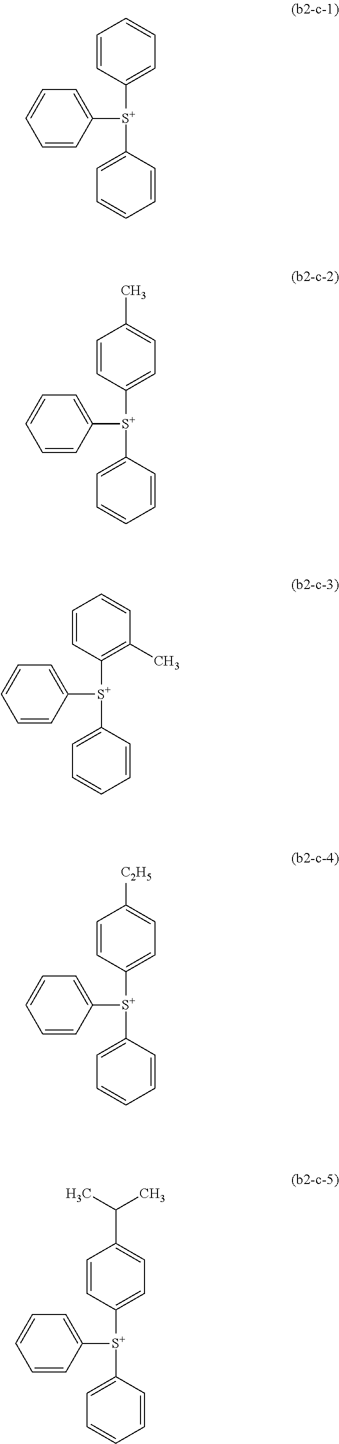 [SMILES: c1ccc([S+](c2ccccc2)c2ccccc2)cc1 ; Cc1ccc([S+](c2ccccc2)c2ccccc2)cc1 ; Cc1ccccc1[S+](c1ccccc1)c1ccccc1 ; CCc1ccc([S+](c2ccccc2)c2ccccc2)cc1 ; CC(C)c1ccc([S+](c2ccccc2)c2ccccc2)cc1]

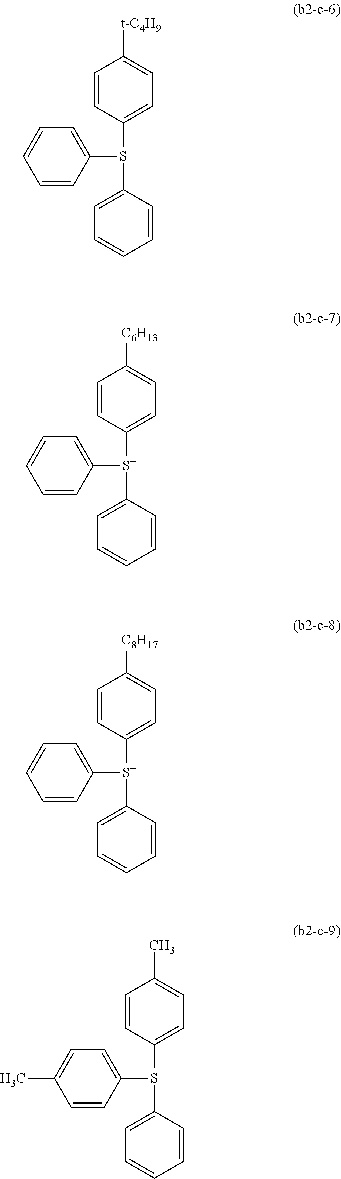

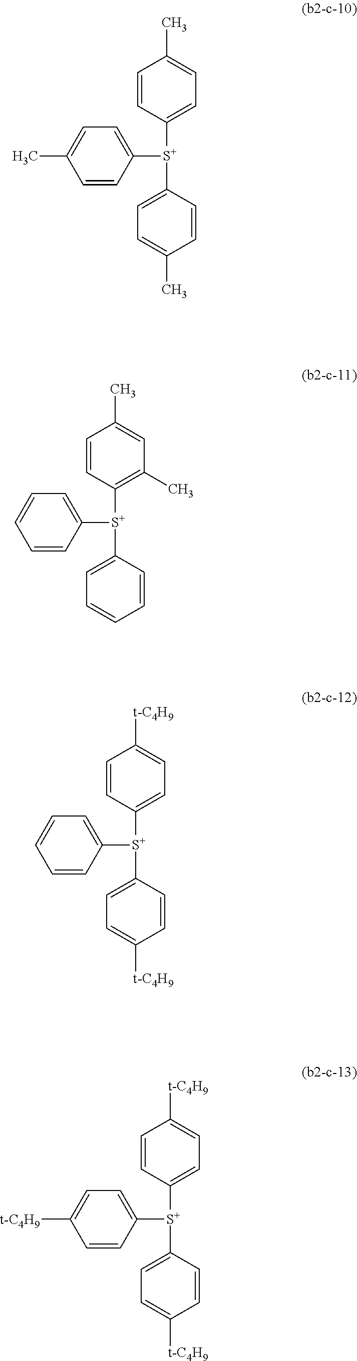

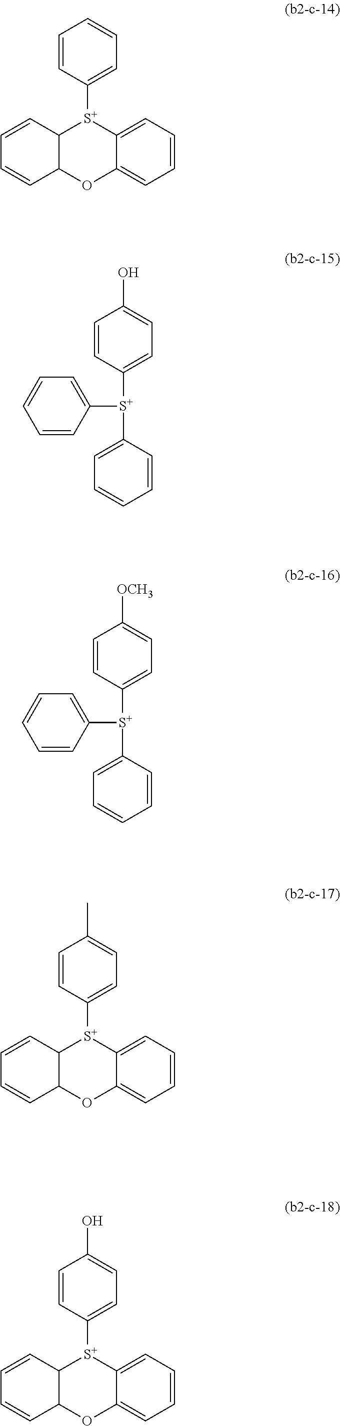 [SMILES: C1=CC2Oc3ccccc3[S+](c3ccccc3)C2C=C1 ; Oc1ccc([S+](c2ccccc2)c2ccccc2)cc1 ; COc1ccc([S+](c2ccccc2)c2ccccc2)cc1 ; Cc1ccc([S+]2c3ccccc3OC3C=CC=CC32)cc1 ; Oc1ccc([S+]2c3ccccc3OC3C=CC=CC32)cc1]

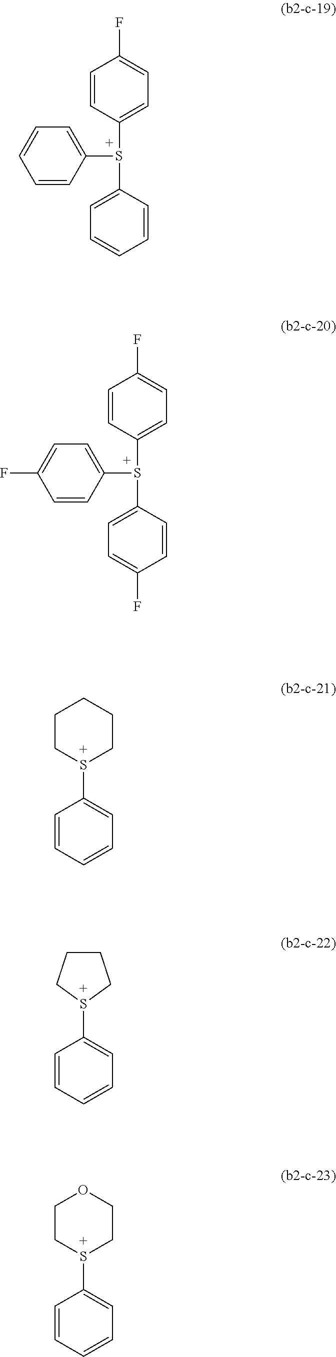

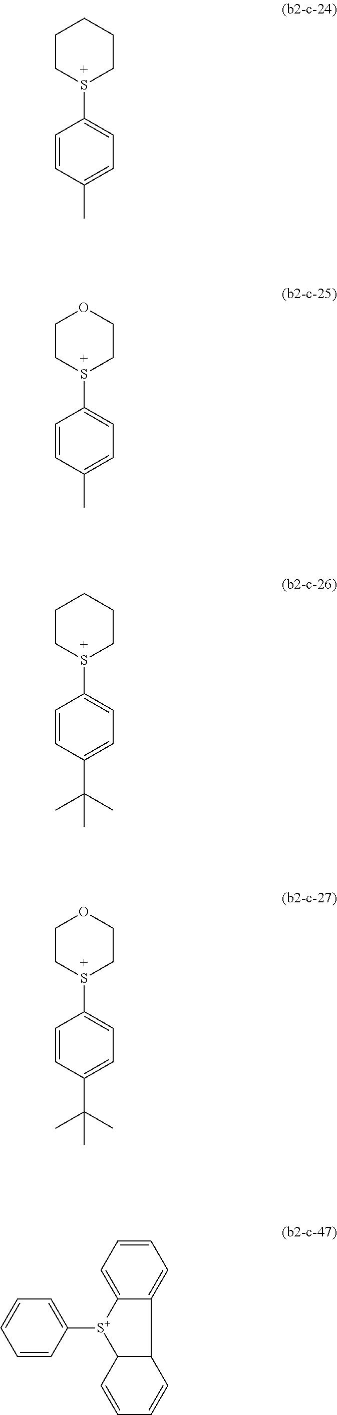

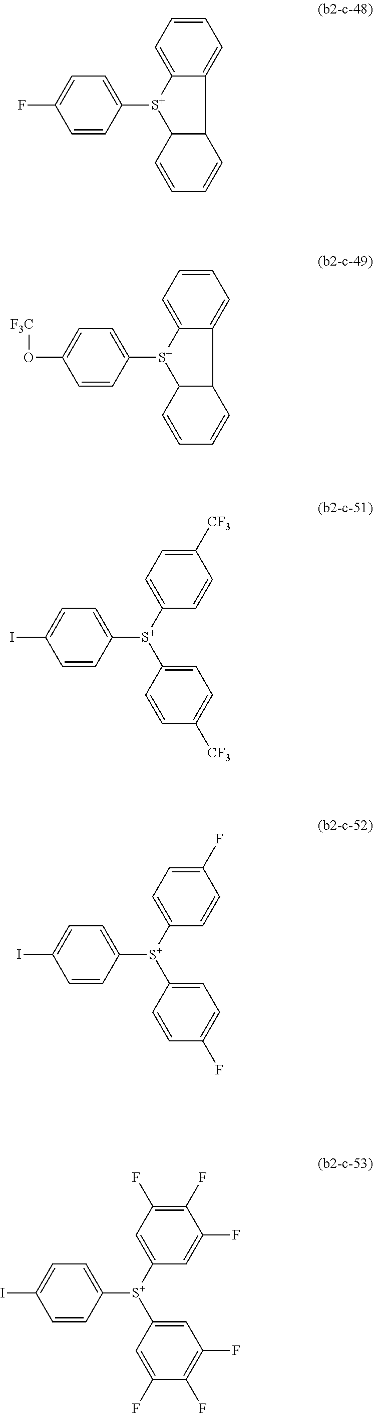 [SMILES: Fc1ccc([S+]2c3ccccc3C3C=CC=CC32)cc1 ; FC(F)(F)Oc1ccc([S+]2c3ccccc3C3C=CC=CC32)cc1 ; FC(F)(F)c1ccc([S+](c2ccc(I)cc2)c2ccc(C(F)(F)F)cc2)cc1 ; Fc1ccc([S+](c2ccc(F)cc2)c2ccc(I)cc2)cc1 ; Fc1cc([S+](c2ccc(I)cc2)c2cc(F)c(F)c(F)c2)cc(F)c1F]

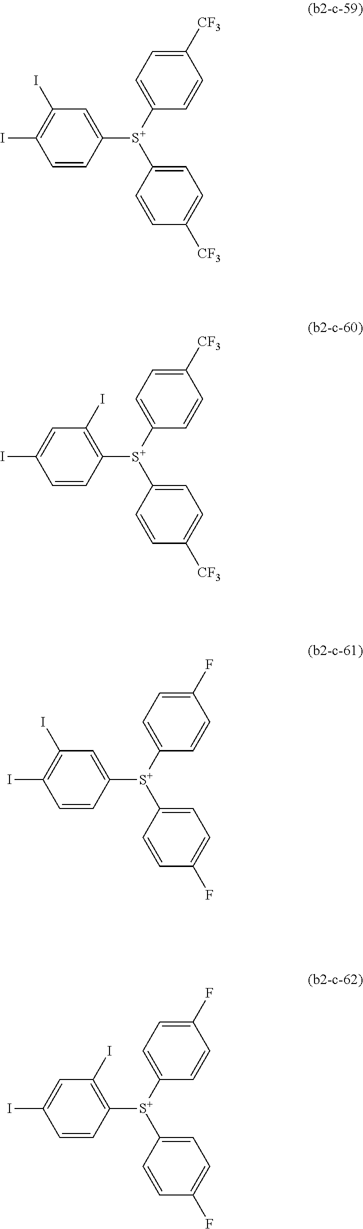 [SMILES: FC(F)(F)c1ccc([S+](c2ccc(C(F)(F)F)cc2)c2ccc(I)c(I)c2)cc1 ; FC(F)(F)c1ccc([S+](c2ccc(C(F)(F)F)cc2)c2ccc(I)cc2I)cc1 ; Fc1ccc([S+](c2ccc(F)cc2)c2ccc(I)c(I)c2)cc1 ; Fc1ccc([S+](c2ccc(F)cc2)c2ccc(I)cc2I)cc1]

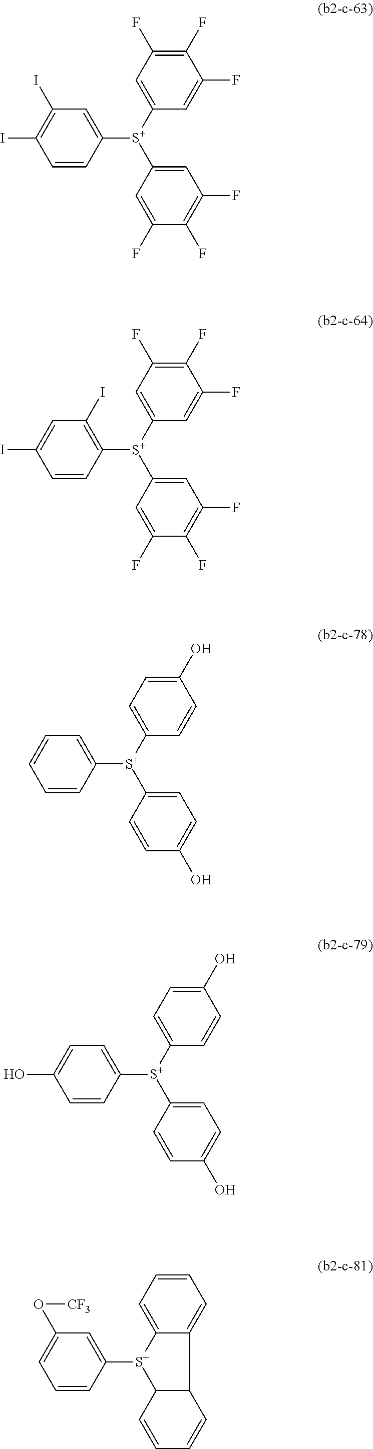 [SMILES: Fc1cc([S+](c2cc(F)c(F)c(F)c2)c2ccc(I)c(I)c2)cc(F)c1F ; Fc1cc([S+](c2cc(F)c(F)c(F)c2)c2ccc(I)cc2I)cc(F)c1F ; Oc1ccc([S+](c2ccccc2)c2ccc(O)cc2)cc1 ; Oc1ccc([S+](c2ccc(O)cc2)c2ccc(O)cc2)cc1 ; FC(F)(F)Oc1cccc([S+]2c3ccccc3C3C=CC=CC32)c1]

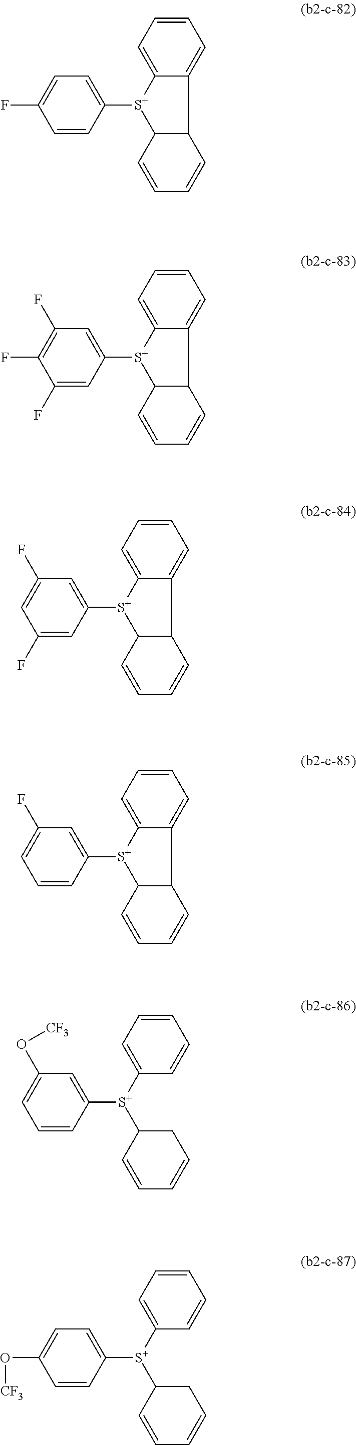 [SMILES: Fc1ccc([S+]2c3ccccc3C3C=CC=CC32)cc1 ; Fc1cc([S+]2c3ccccc3C3C=CC=CC32)cc(F)c1F ; Fc1cc(F)cc([S+]2c3ccccc3C3C=CC=CC32)c1 ; Fc1cccc([S+]2c3ccccc3C3C=CC=CC32)c1 ; FC(F)(F)Oc1cccc([S+](c2ccccc2)C2C=CC=CC2)c1 ; FC(F)(F)Oc1ccc([S+](c2ccccc2)C2C=CC=CC2)cc1]

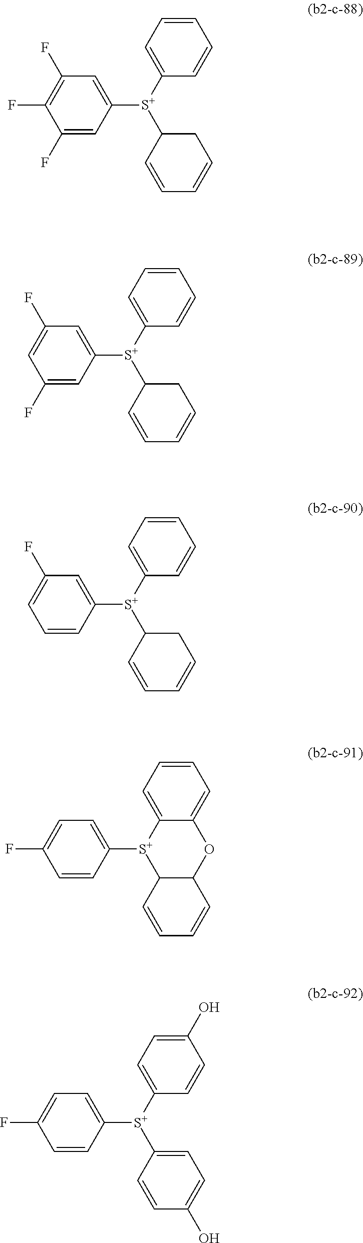 [SMILES: Fc1cc([S+](c2ccccc2)C2C=CC=CC2)cc(F)c1F ; Fc1cc(F)cc([S+](c2ccccc2)C2C=CC=CC2)c1 ; Fc1cccc([S+](c2ccccc2)C2C=CC=CC2)c1 ; Fc1ccc([S+]2c3ccccc3OC3C=CC=CC32)cc1 ; Oc1ccc([S+](c2ccc(O)cc2)c2ccc(F)cc2)cc1]

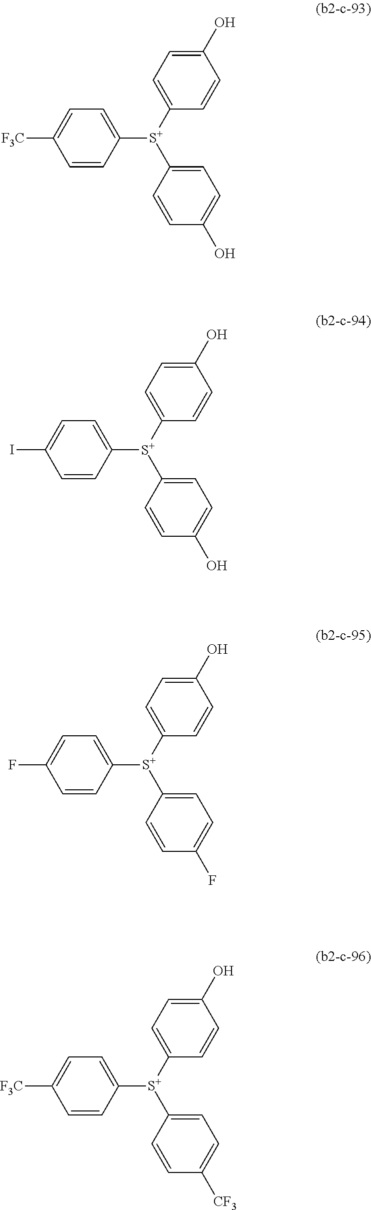 [SMILES: Oc1ccc([S+](c2ccc(O)cc2)c2ccc(C(F)(F)F)cc2)cc1 ; Oc1ccc([S+](c2ccc(O)cc2)c2ccc(I)cc2)cc1 ; Oc1ccc([S+](c2ccc(F)cc2)c2ccc(F)cc2)cc1 ; Oc1ccc([S+](c2ccc(C(F)(F)F)cc2)c2ccc(C(F)(F)F)cc2)cc1]

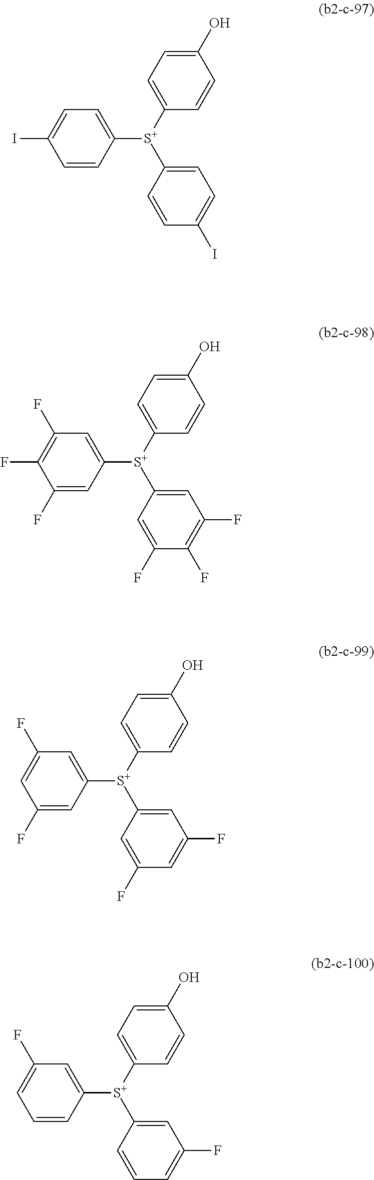 [SMILES: Oc1ccc([S+](c2ccc(I)cc2)c2ccc(I)cc2)cc1 ; Oc1ccc([S+](c2cc(F)c(F)c(F)c2)c2cc(F)c(F)c(F)c2)cc1 ; Oc1ccc([S+](c2cc(F)cc(F)c2)c2cc(F)cc(F)c2)cc1 ; Oc1ccc([S+](c2cccc(F)c2)c2cccc(F)c2)cc1]

Examples of the cation (b2-1) include the following cations.

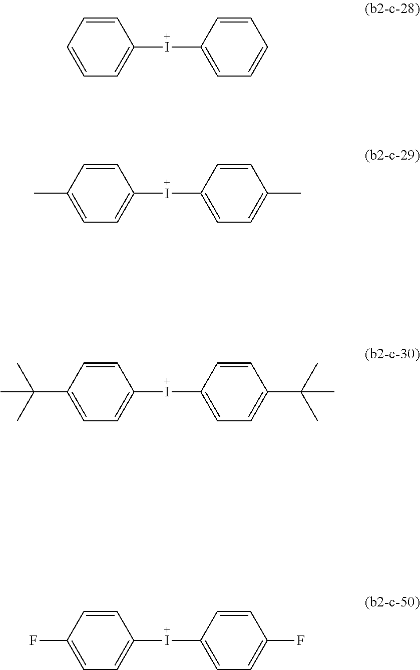

Examples of the cation (b2-3) include the following cations.

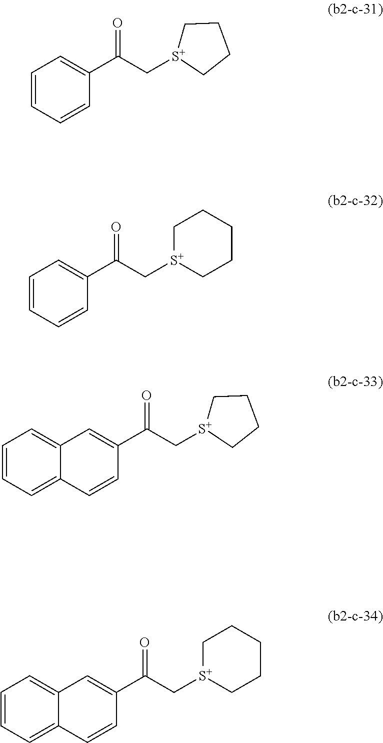 [SMILES: O=C(C[S+]1CCCC1)c1ccccc1 ; O=C(C[S+]1CCCCC1)c1ccccc1 ; O=C(C[S+]1CCCC1)c1ccc2ccccc2c1 ; O=C(C[S+]1CCCCC1)c1ccc2ccccc2c1]

Examples of the cation (b2-4) include the following cations.

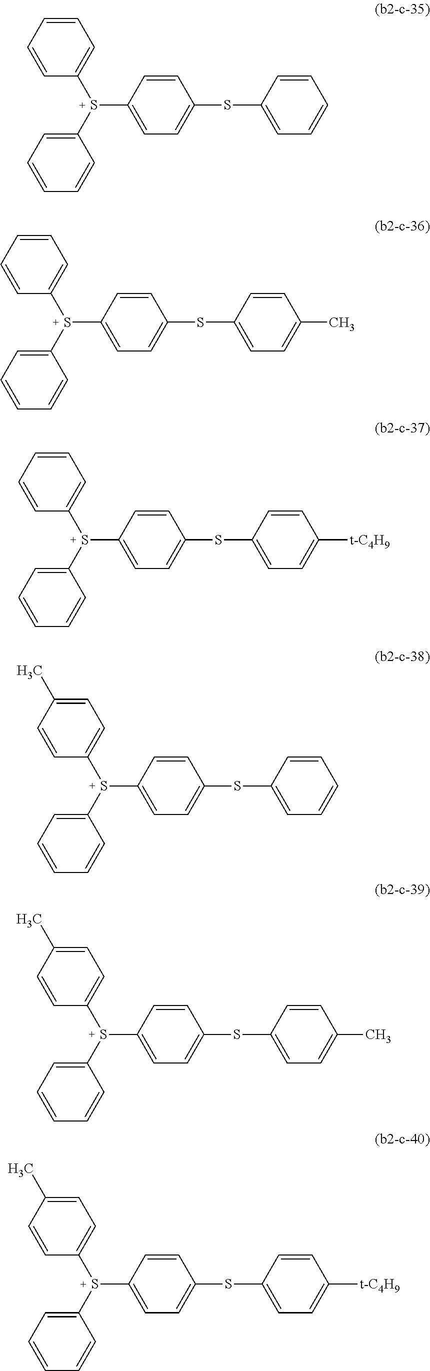

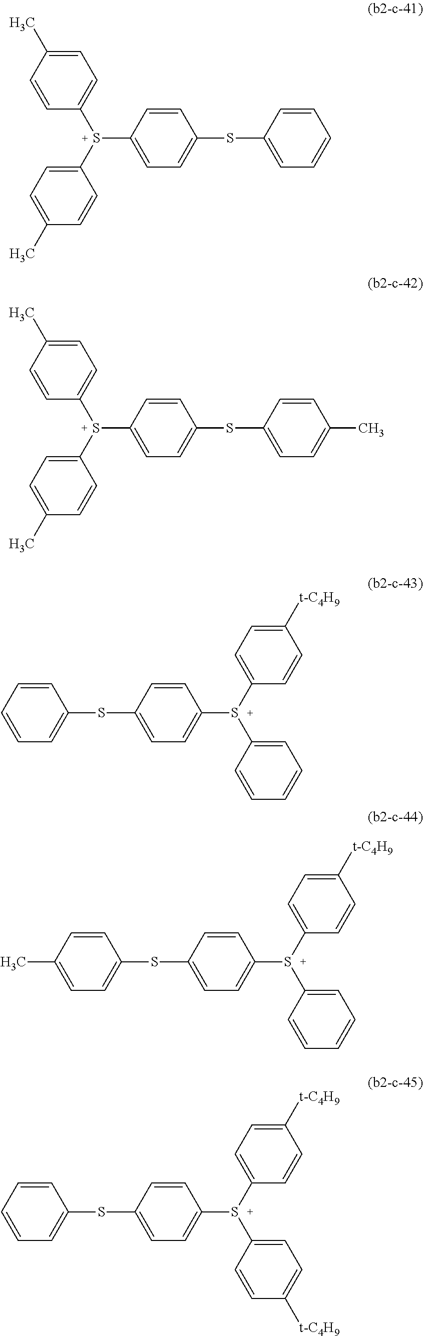

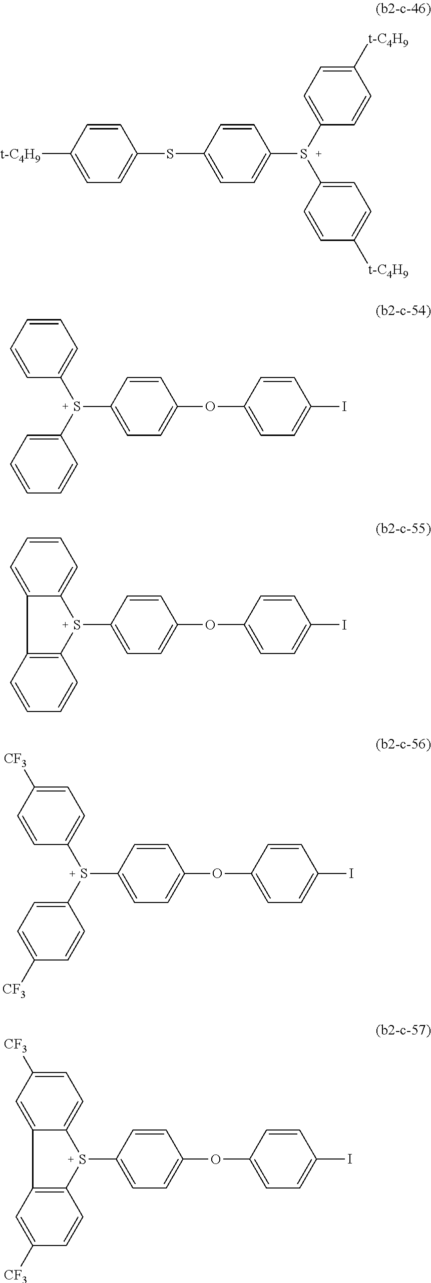

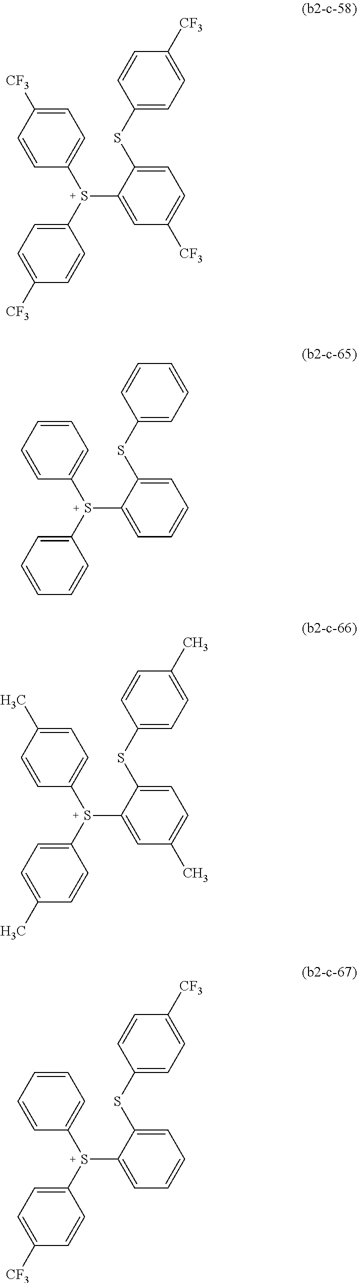

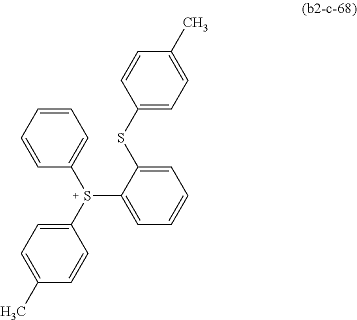

Examples of the cation (b2-5) include the following cations.

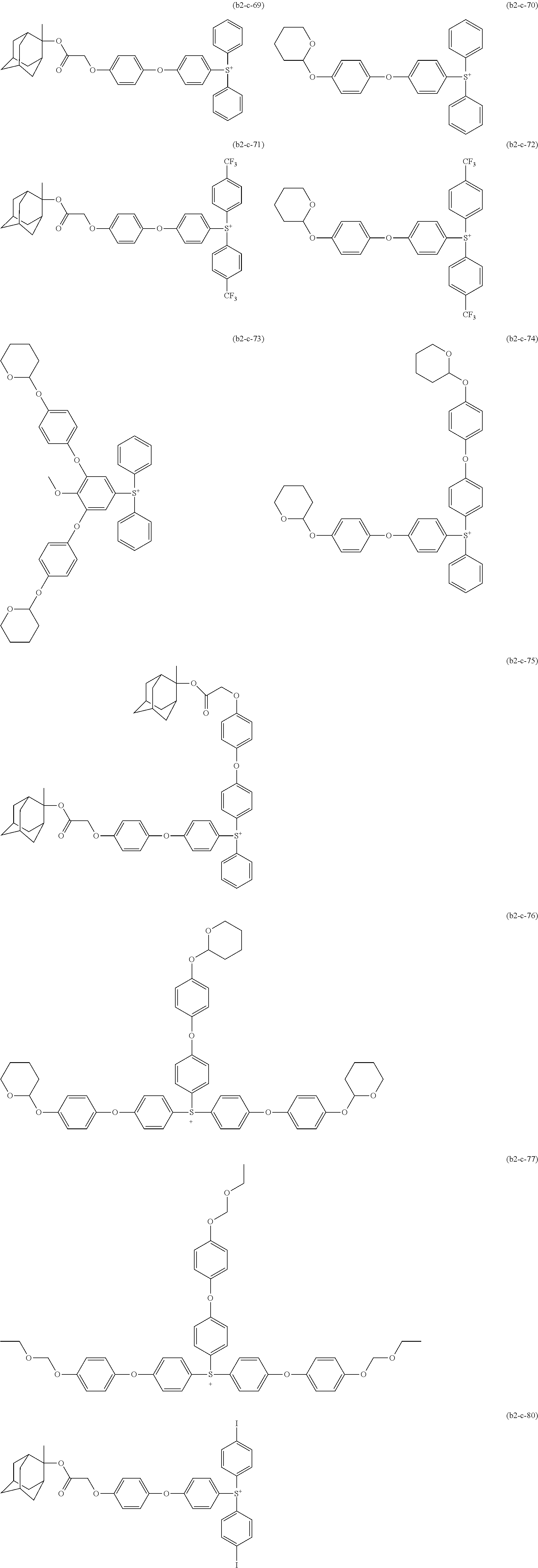

The compound (B) is a combination of the above-described anions and the above-described organic cations, and these anions and cations can be arbitrarily combined. Preferred examples of the compound (B) include a combination of an anion represented by any one of formula (B1a-1) to formula (B1a-4), formula (B1a-7) to formula (B1a-11), formula (B1a-14) to formula (B1a-30), formula (B1a-35) to formula (B1a-70), formula (B2a-1) to formula (B2a-11), formula (B2a-16) to formula (B2a-20), formula (B3a-1) to formula (B3a-3), formula (B3a-11) to formula (B3a-14), formula (B4a-1) to formula (B4a-4), and formula (B4a-11) to formula (B4a-14), with a cation (b2-1), a cation (b2-2), a cation (b2-3), a cation (b2-4) or a cation (b2-5).

Preferred examples of the compound (B) include the respective compounds represented by formula (B1-1) to formula (B1-106), formula (B2-1) to formula (B2-20), formula (B3-1) to formula (B3-28), and formula (B4-1) to formula (B4-16). Among the compounds, a compound containing an arylsulfonium cation is preferable, and the compounds are particularly preferable that are represented by formula (B1-1) to formula (B1-3), formula (B1-5) to formula (B1-7), formula (B1-11) to formula (B1-14), formula (B1-20) to formula (B1-26), formula (B1-29), formula (B1-31) to formula (B1-106), formula (B2-1) to formula (B2-20), formula (B3-1) to formula (B3-28), and formula (B4-1) to formula (B4-16).

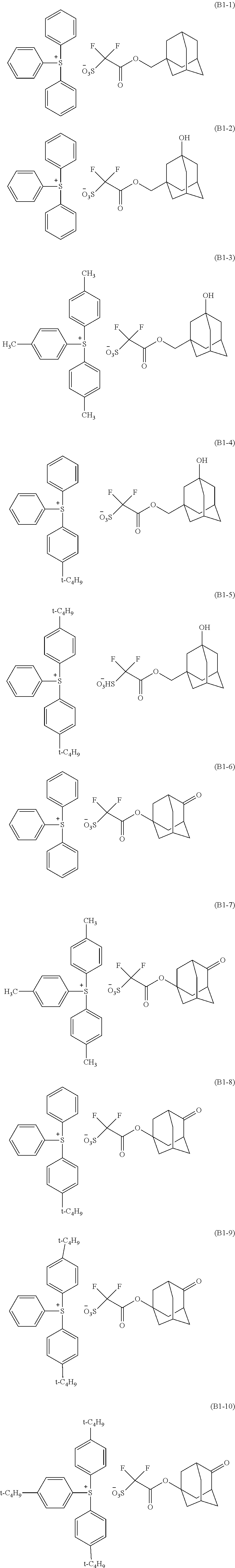

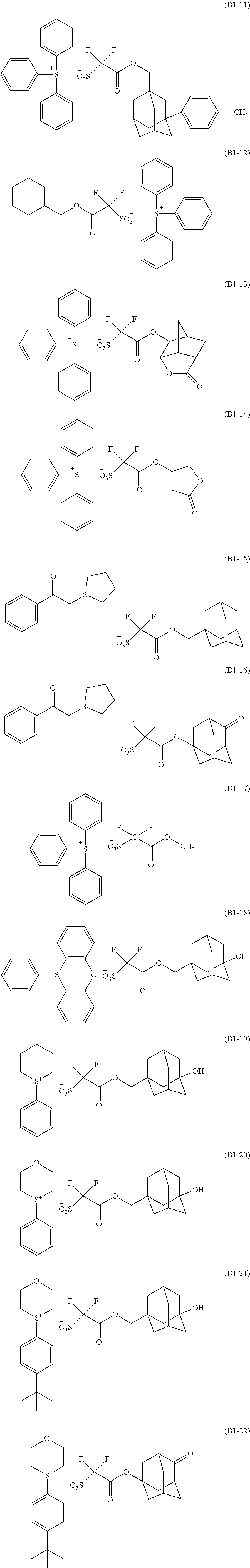

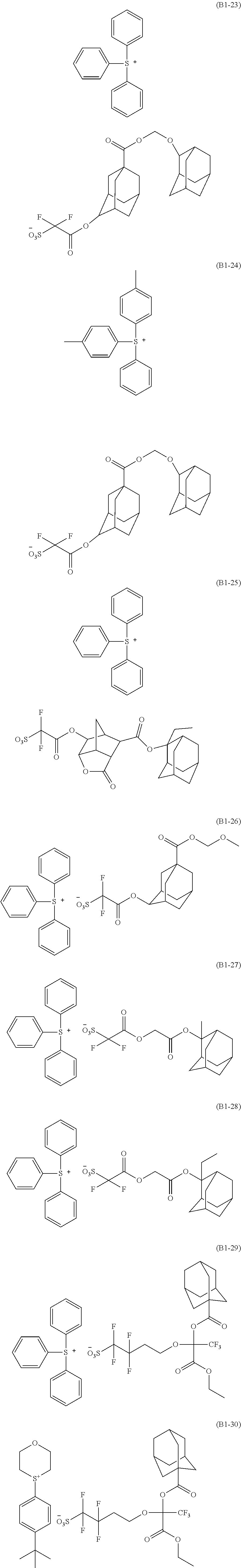

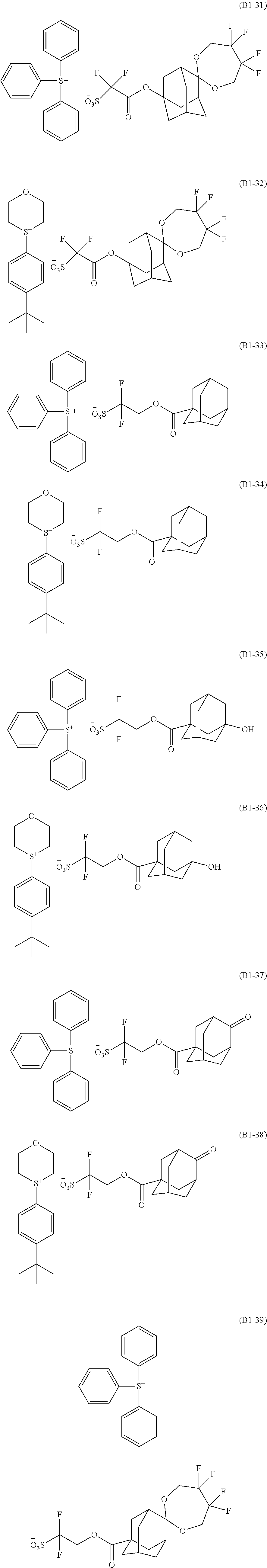

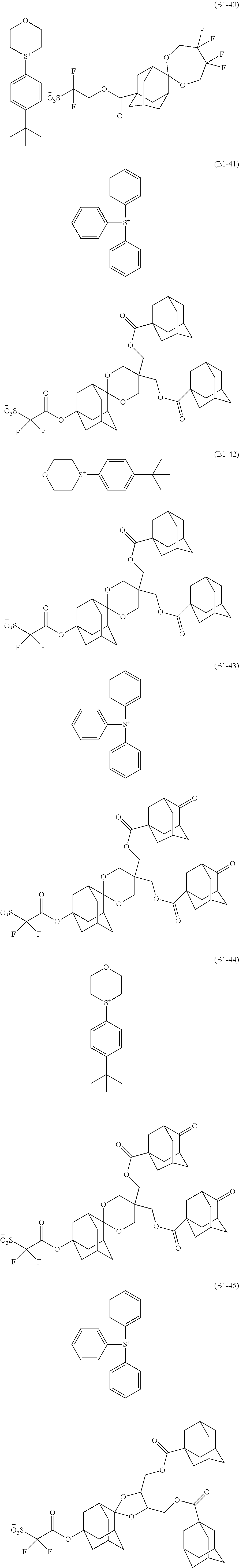

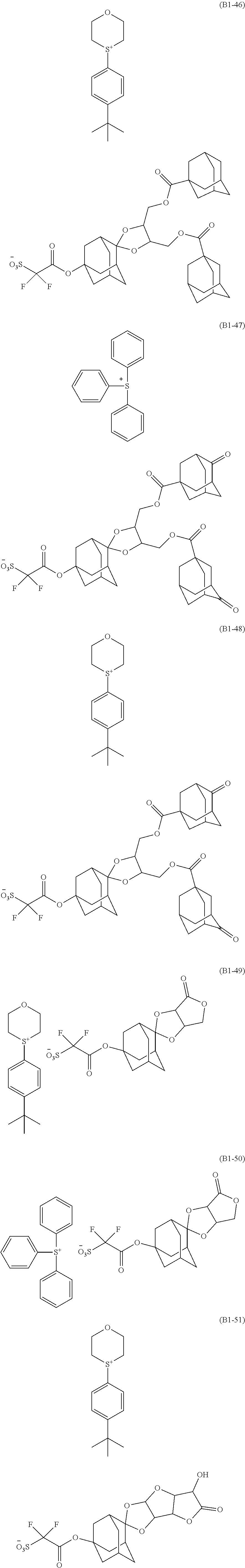

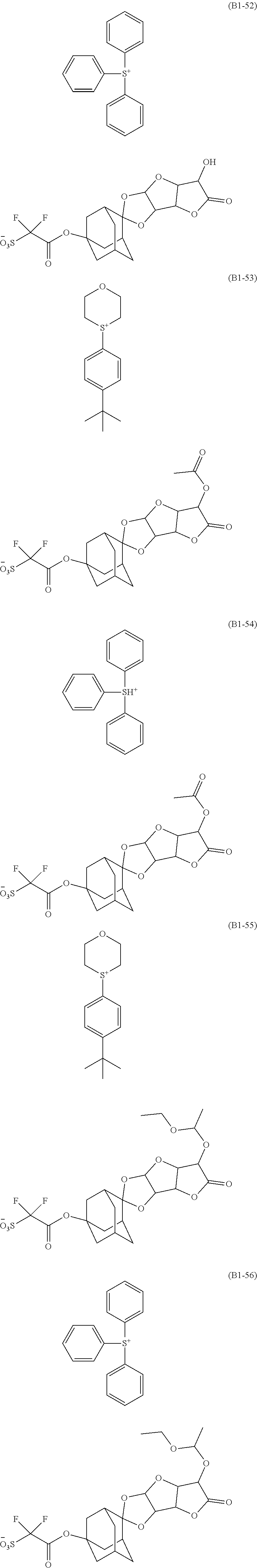

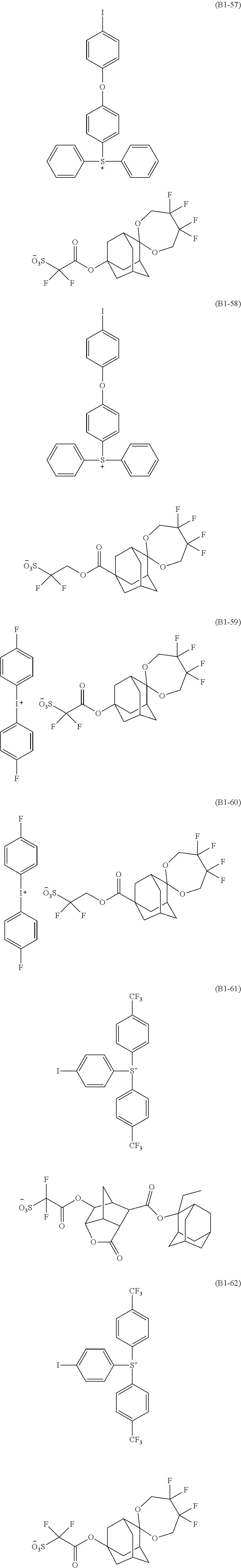

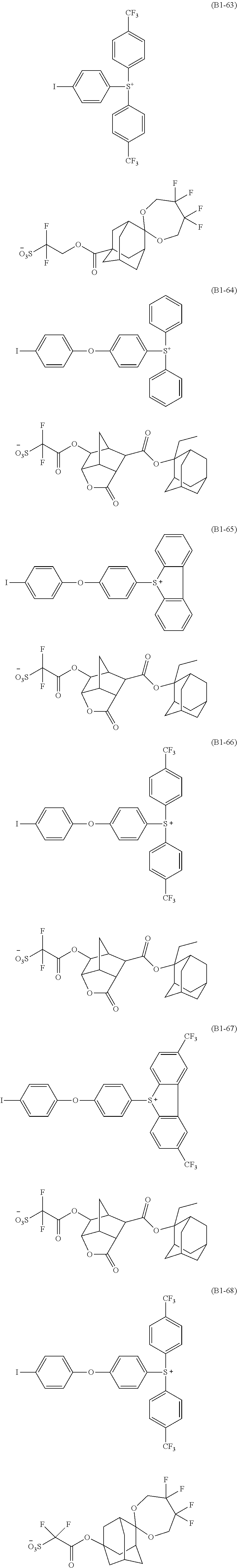

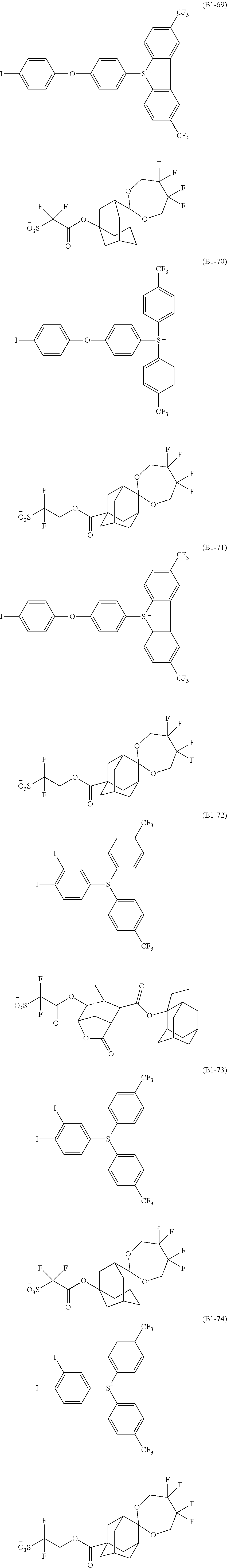

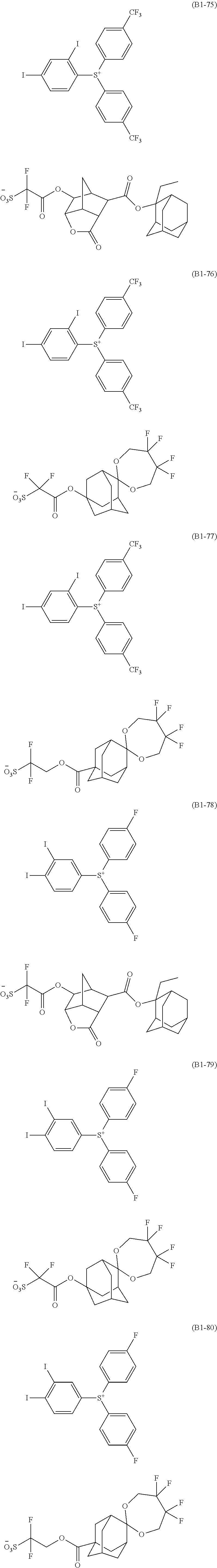

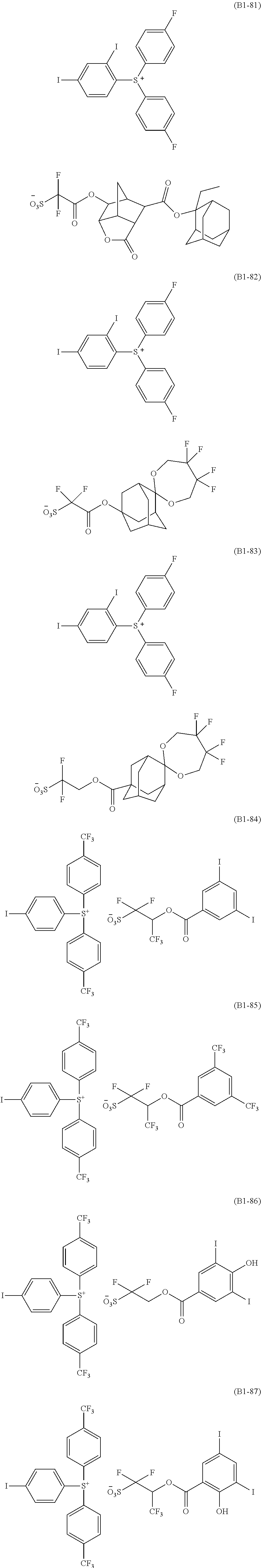

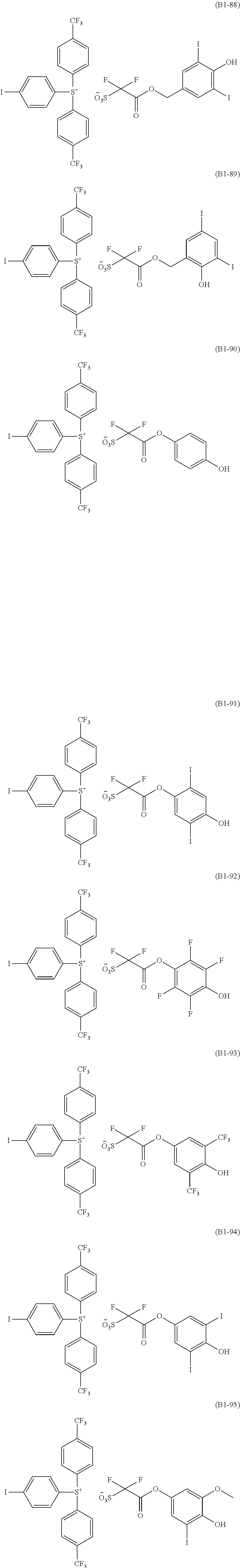

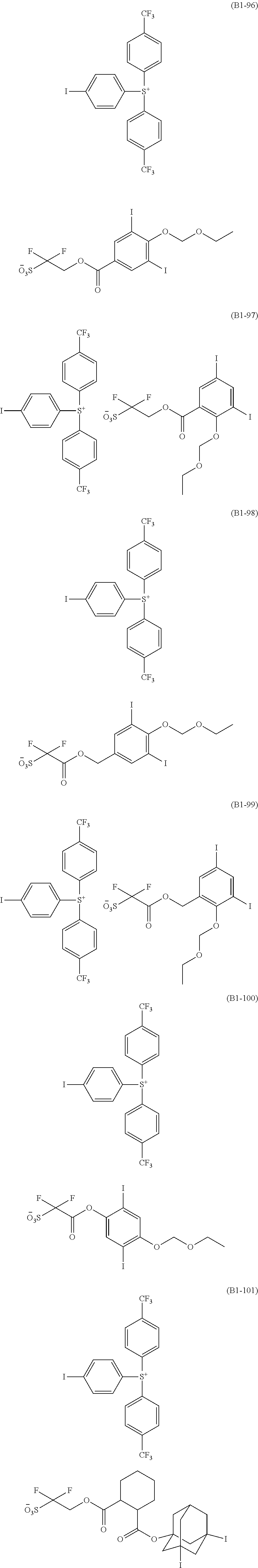

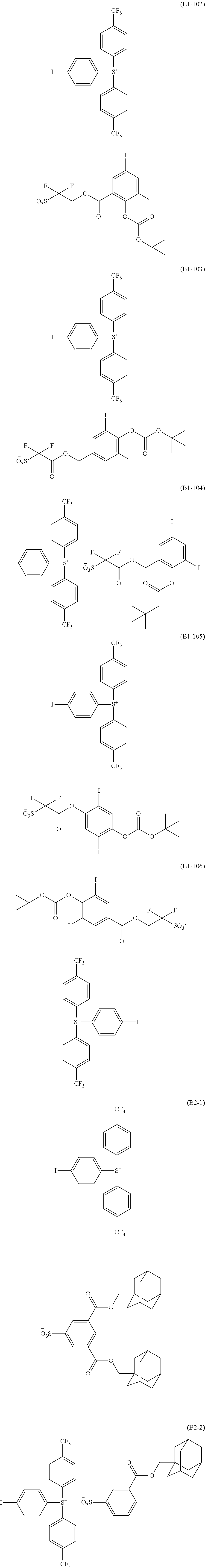

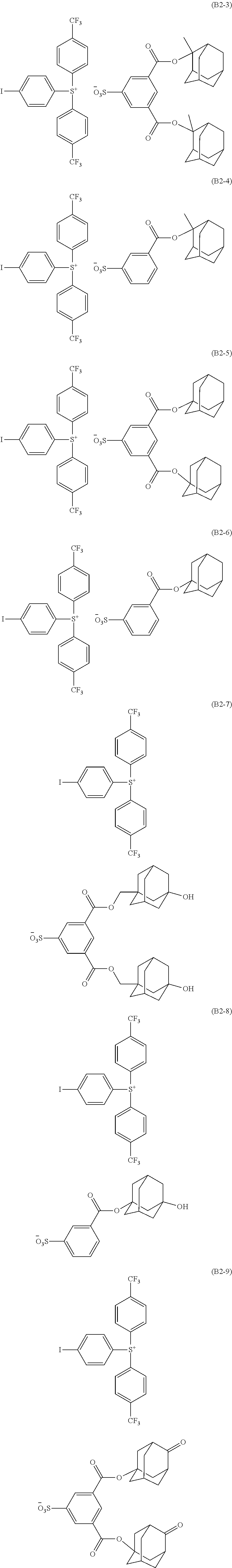

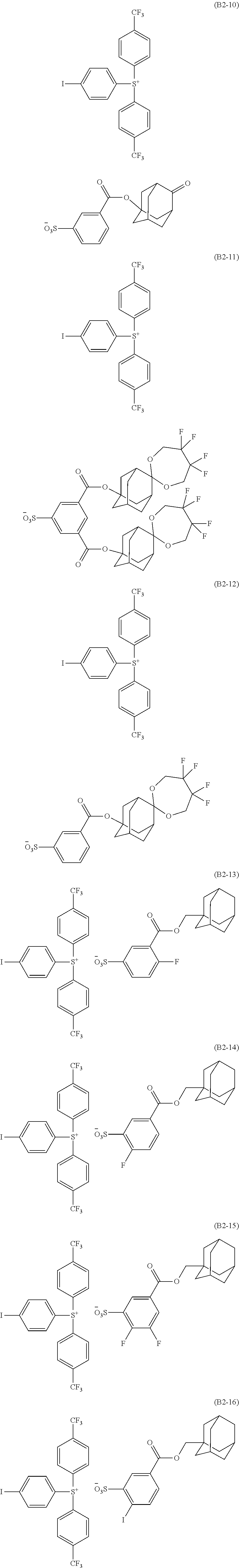

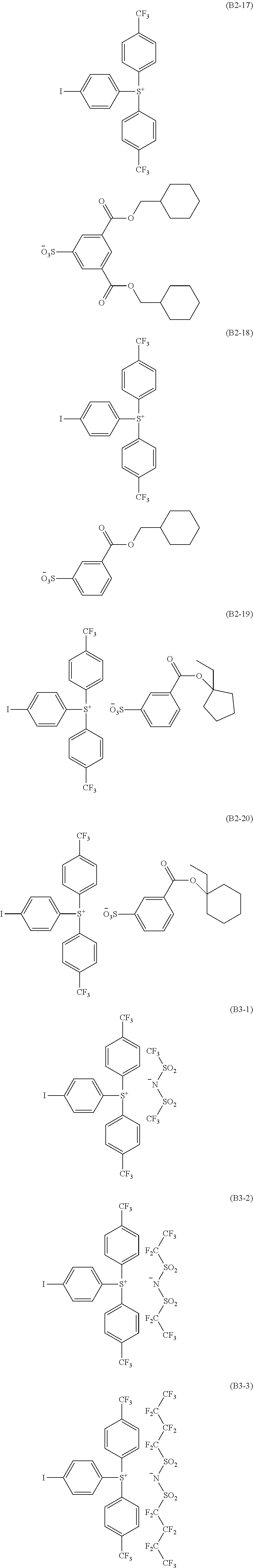

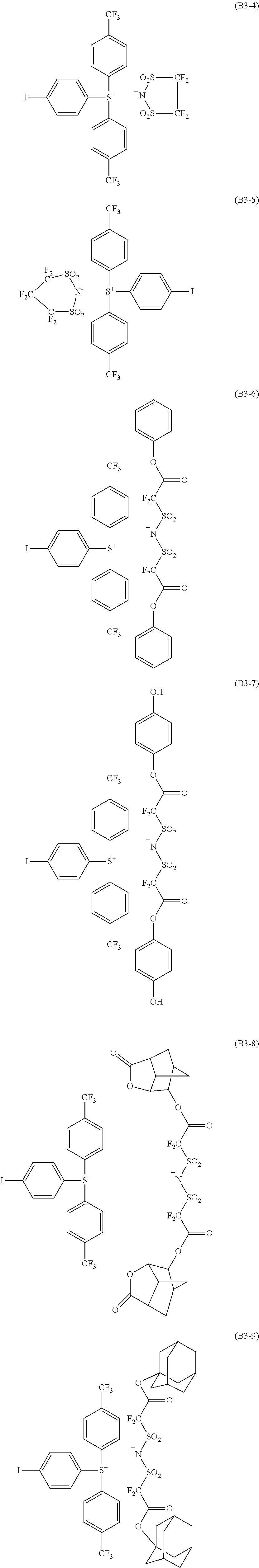

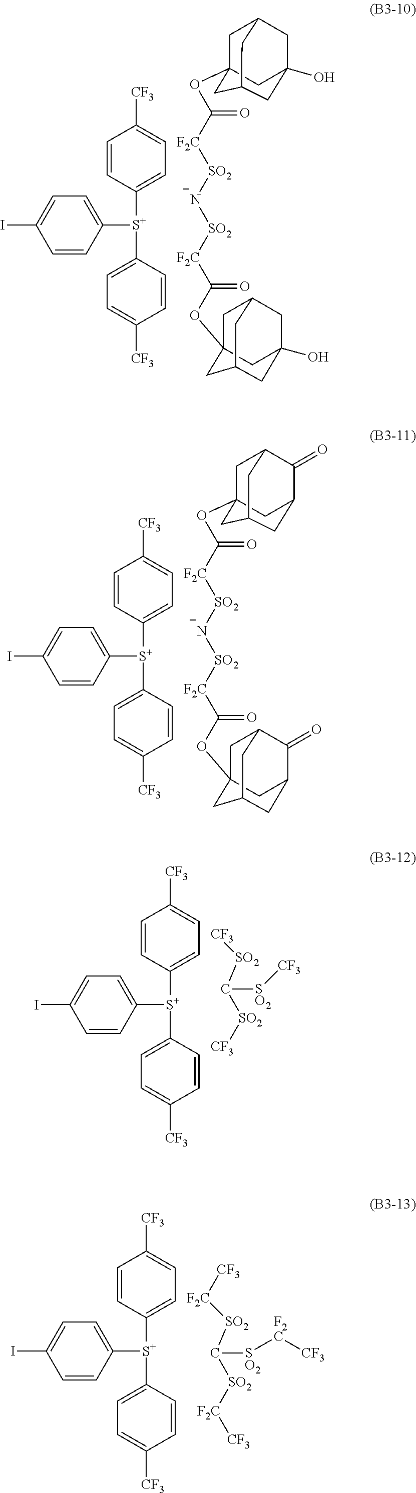

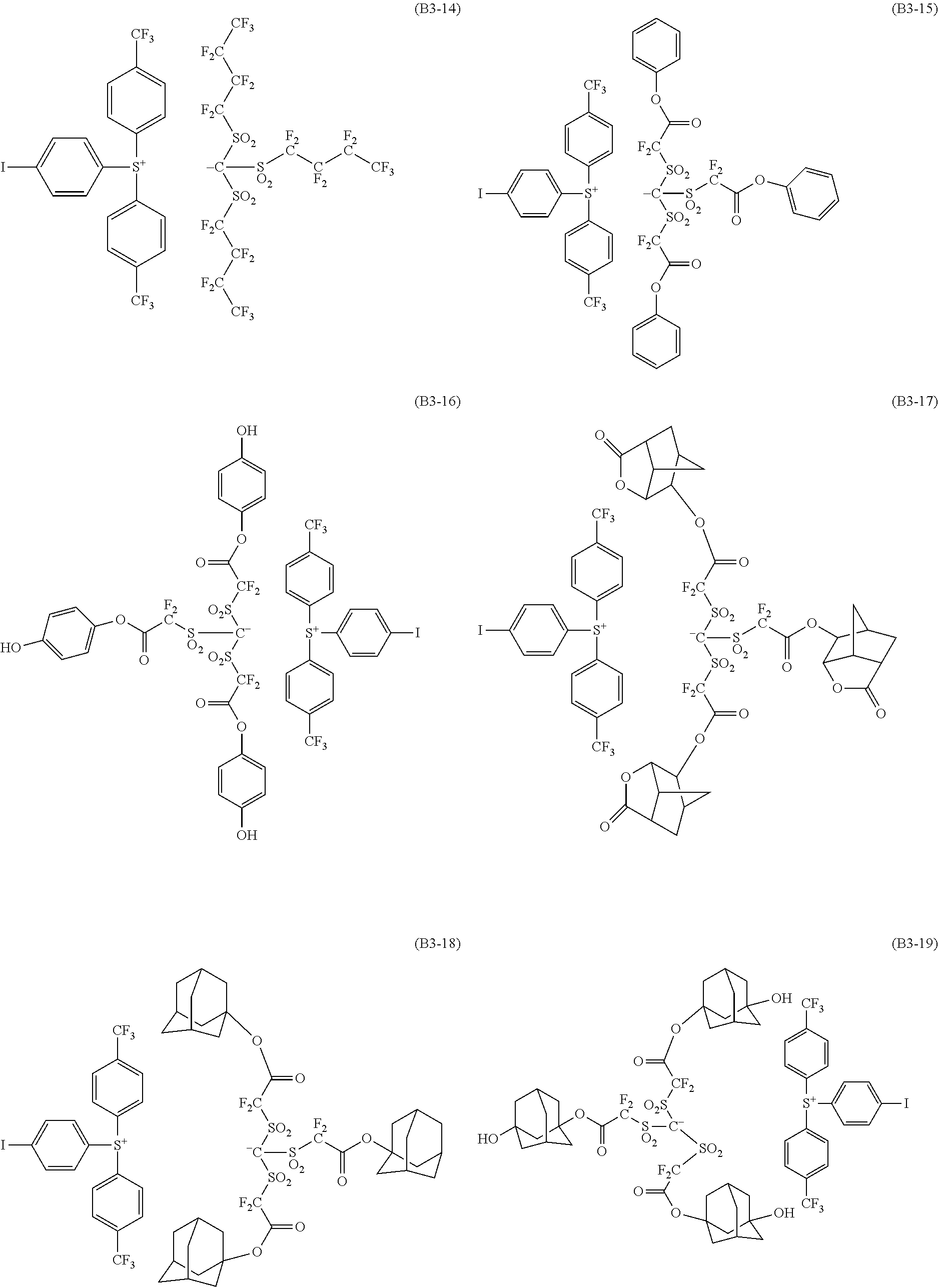

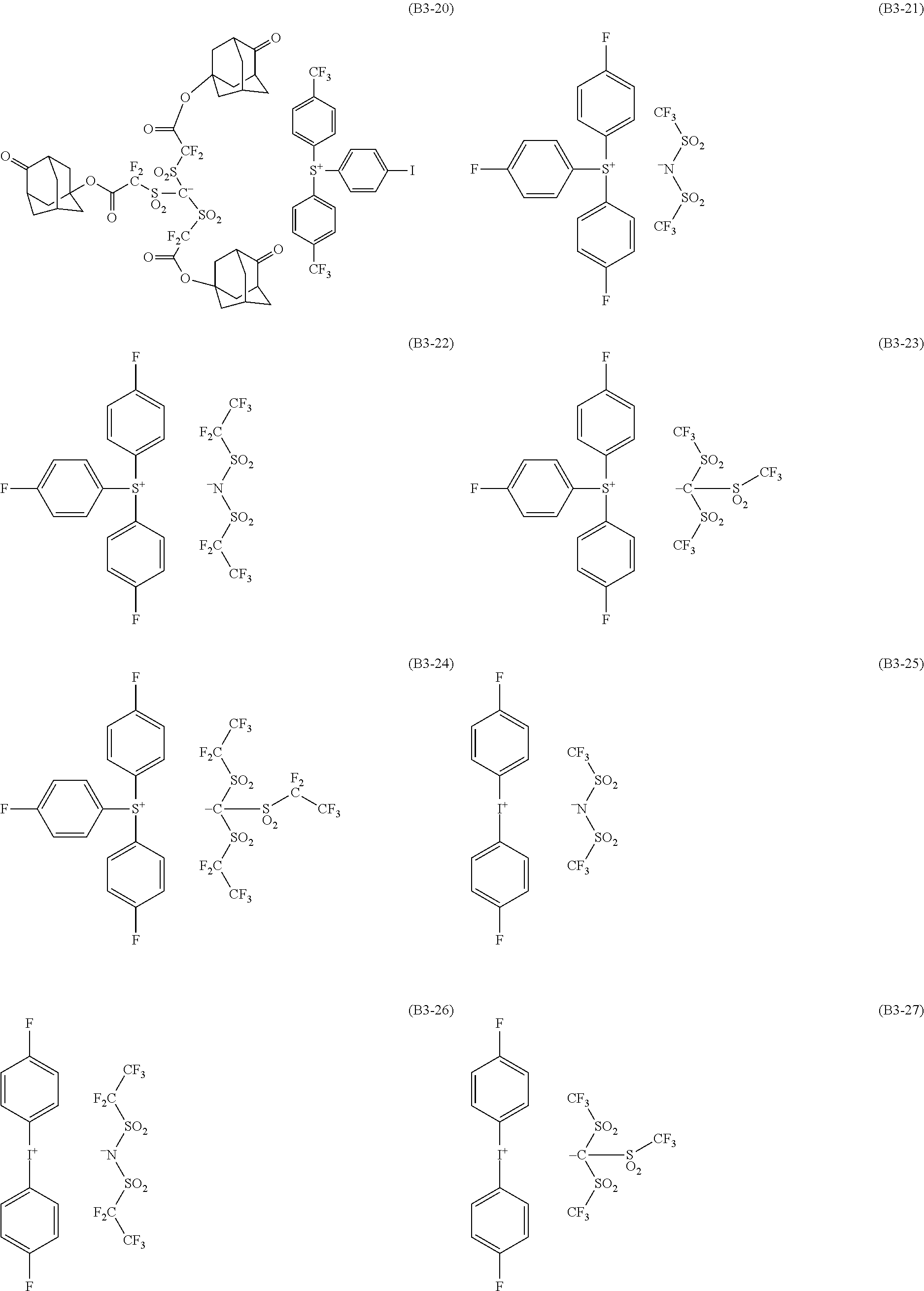

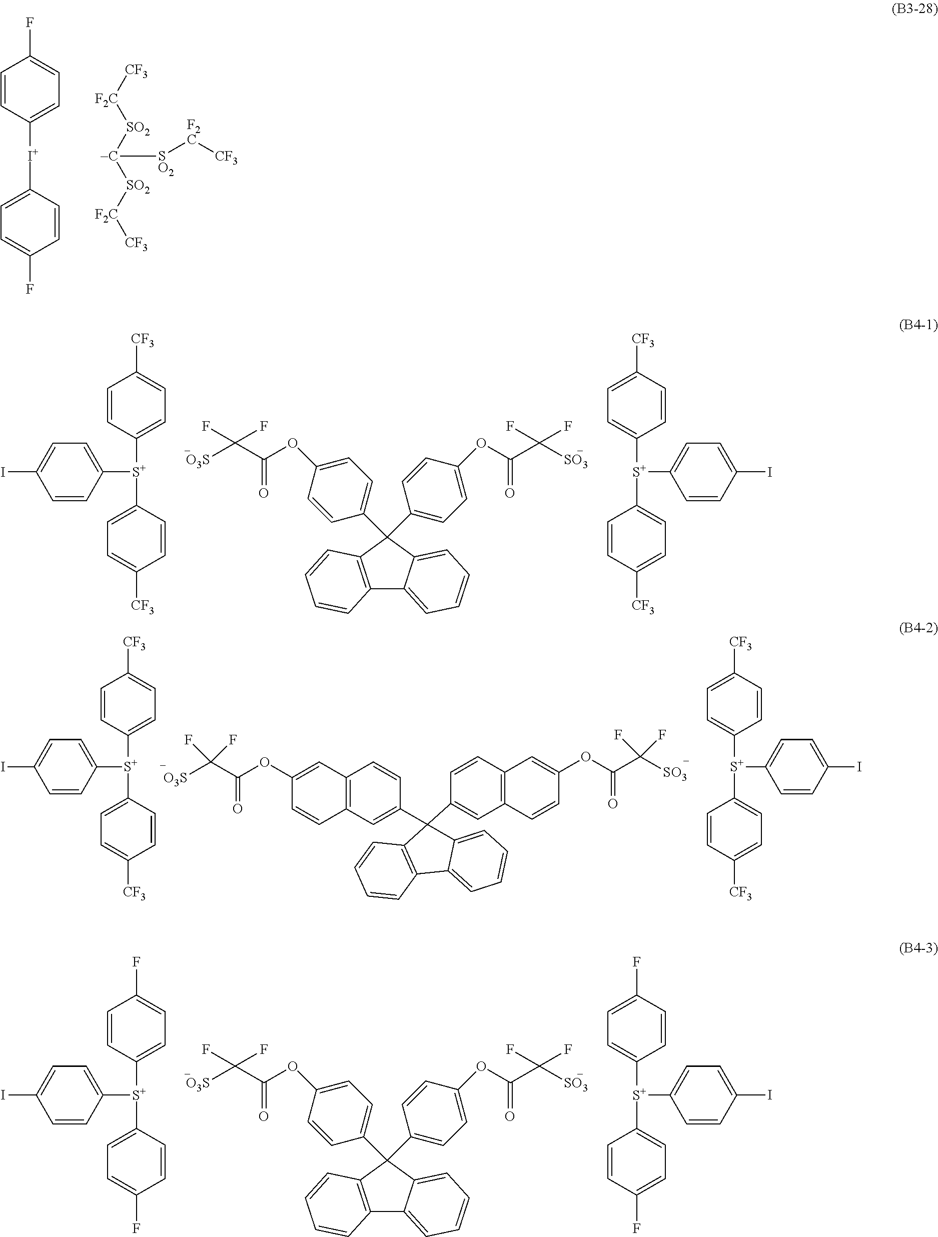

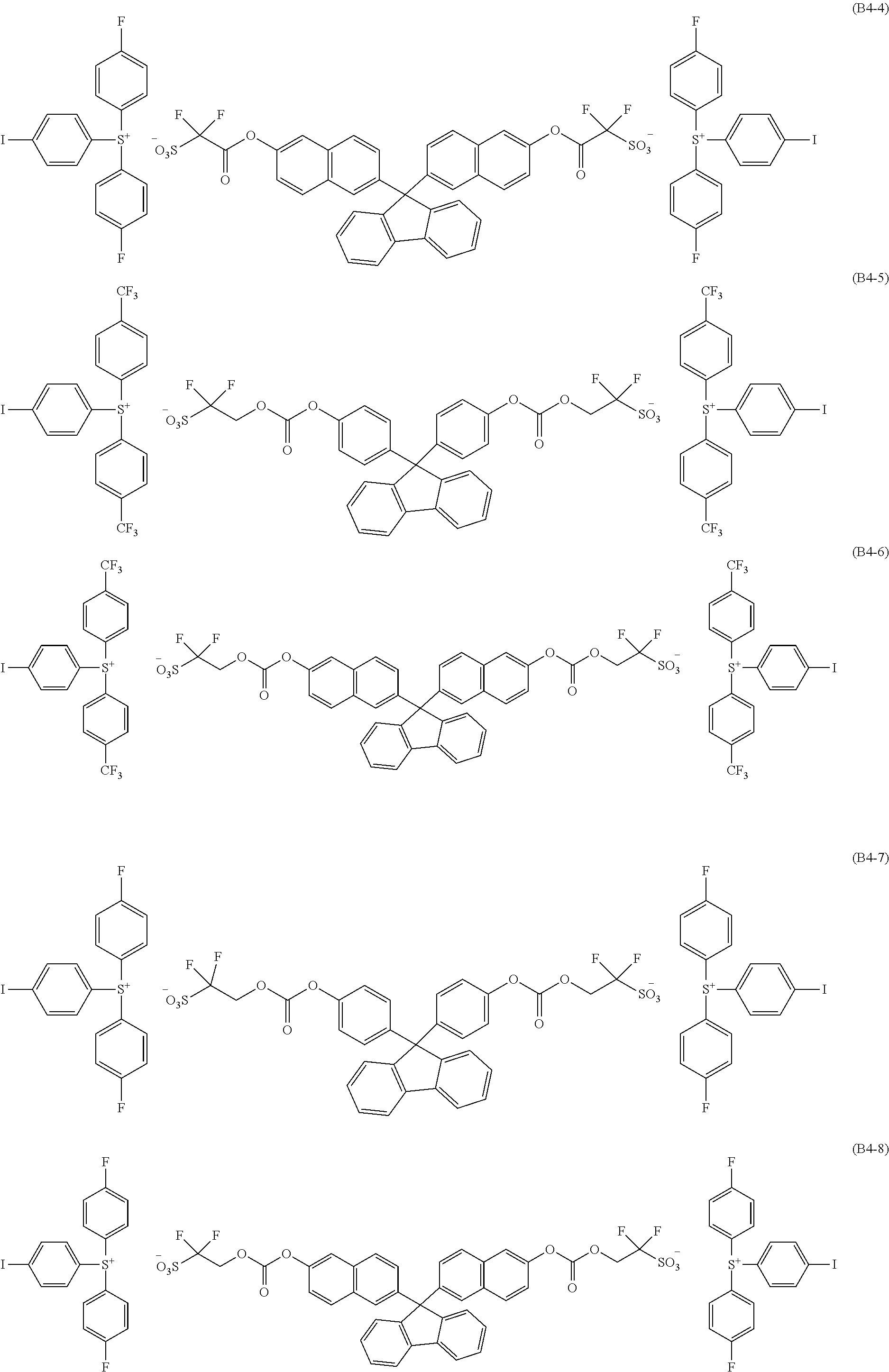

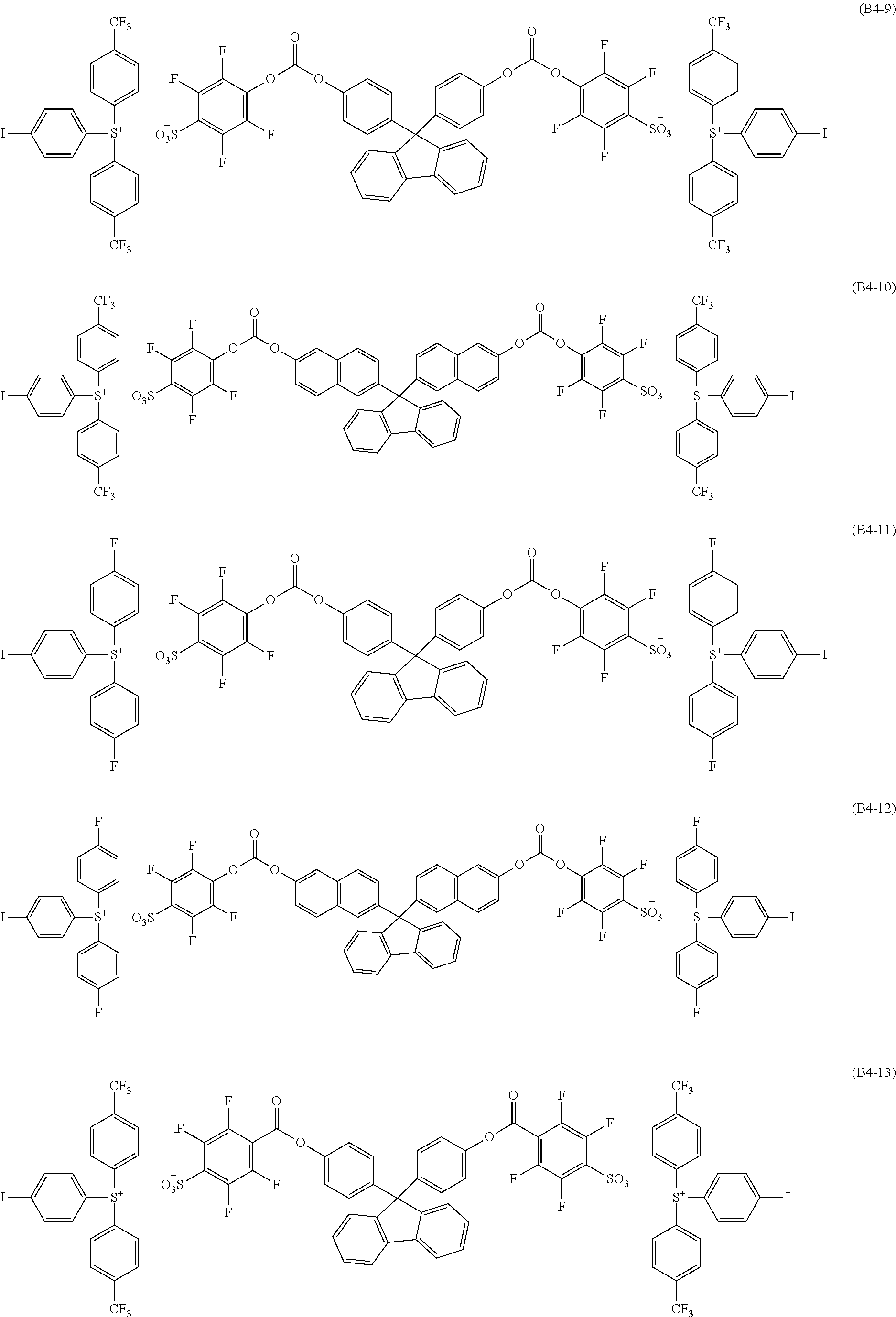

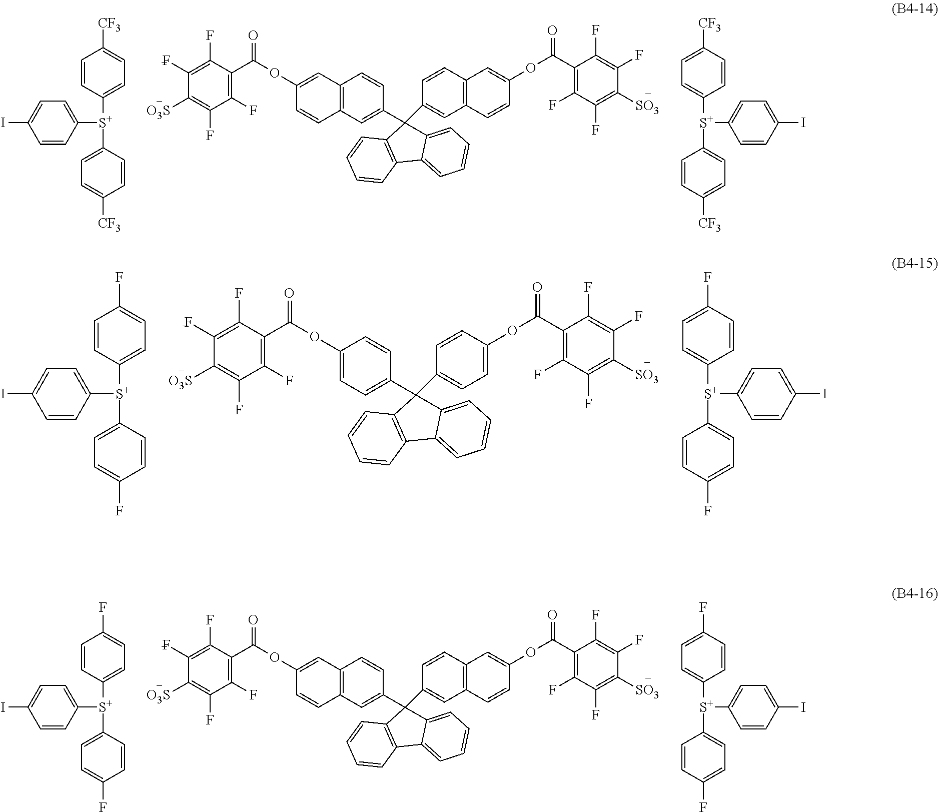

In the resist composition of the present invention, the content ratio of the salt (I) is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and still further preferably 3% by mass or more and 40% by mass or less, with respect to the solid content of the resist composition. In a case where the resist composition contains the resin (A) and/or the like, the content ratio of the salt (I) is preferably 1 part by mass or more and 45 parts by mass or less, more preferably 1 part by mass or more and 40 parts by mass or less, and further preferably 3 parts by mass or more and 40 parts by mass or less, with respect to 100 parts by mass of the resin (A) and/or the like.

In the resist composition of the present invention, the content ratio of the structural unit (IP) is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and still further preferably 3% by mass or more and 40% by mass or less, with respect to the solid content of the resist composition.

In the resist composition of the present invention, the content ratio of the compound (B) is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and still further preferably 3% by mass or more and 40% by mass or less, with respect to the solid content of the resist composition. In a case where the resist composition contains the resin (A) and/or the like, the content ratio of the compound (B) is preferably 1 part by mass or more and 45 parts by mass or less, more preferably 1 part by mass or more and 40 parts by mass or less, and further preferably 3 parts by mass or more and 40 parts by mass or less, with respect to 100 parts by mass of the resin (A) and/or the like.

In the resist composition of the present invention, the total content ratio of the acid generating agents is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and still further preferably 3% by mass or more and 40% by mass or less, with respect to the solid content of the resist composition. In a case where the resist composition contains the resin (A) and/or the like, the total content ratio of the acid generating agents is preferably 1 part by mass or more and 45 parts by mass or less, more preferably 1 part by mass or more and 40 parts by mass or less, and further preferably 3 parts by mass or more and 40 parts by mass or less, with respect to 100 parts by mass of the resin (A) and/or the like.

### <Solvent (E)>

The content ratio of the solvent (E) in the resist composition is typically 90% by mass or more and 99.9% by mass or less, preferably 92% by mass or more and 99% by mass or less, and more preferably 94% by mass or more and 99% by mass or less. The content ratio of the solvent (E) can be measured by known analytical means such as liquid chromatography or gas chromatography.

Examples of the solvent (E) include: a glycol ether ester such as ethyl cellosolve acetate, methyl cellosolve acetate and propylene glycol monomethyl ether acetate; a glycol ether such as propylene glycol monomethyl ether; an ester such as ethyl lactate, butyl acetate, amyl acetate and ethyl pyruvate; a ketone such as acetone, methyl isobutyl ketone, 2-heptanone and cyclohexanone; a cyclic ester such as γ-butyrolactone. The solvent (E) may be used singly or in combination of two or more.

### <Quencher (C)>

Examples of the quencher (C) include a salt that generates an acid weaker in acidity than the acid generated from a basic nitrogen-containing organic compound and an acid generating agent (salt (I), structural unit (IP) or compound (B)). When the resist composition contains the quencher (C), the content of the quencher (C) is preferably about 0.01 to 15% by mass, more preferably about 0.01 to 10% by mass, further preferably about 0.1 to 8% by mass, and further more preferably about 0.1 to 7% by mass, with respect to the solid content of the resist composition.

Examples of the basic nitrogen-containing organic compound include an amine and an ammonium salt. Examples of the amine include an aliphatic amine and an aromatic amine. Examples of the aliphatic amine include a primary amine, a secondary amine and a tertiary amine.

Examples of the amine include 1-naphthylamine, 2-naphthylamine, aniline, diisopropylaniline, 2-, 3- or 4-methylaniline, 4-nitroaniline, N-methylaniline, N,N-dimethylaniline, diphenylamine, hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethyldipentylamine, ethyldihexylamine, ethyldiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane, 4,4′-diamino-3,3′-diethyldiphenylmethane, 2,2′-methylenebisaniline, imidazole, 4-methylimidazole, pyridine, 4-methylpyridine, 1,2-di(2-pyridyl)ethane, 1,2-di(4-pyridyl)ethane, 1,2-di(2-pyridyl)ethene, 1,2-di(4-pyridyl)ethene, 1,3-di(4-pyridyl)propane, 1,2-di(4-pyridyloxy)ethane, di(2-pyridyl)ketone, 4,4′-dipyridyl sulfide, 4,4′-dipyridyl disulfide, 2,2′-dipyridylamine, 2,2′-dipicolylamine and bipyridine, with diisopropylaniline being preferable, and with 2,6-diisopropyl aniline being more preferable.

Examples of the ammonium salt include tetramethylammonium hydroxide, tetraisopropylammonium hydroxide, tetrabutylammonium hydroxide, tetrahexyl ammonium hydroxide, tetraoctyl ammonium hydroxide, phenyltrimethylammonium hydroxide, 3-(trifluoromethyl)phenyltrimethylammonium hydroxide, tetra-n-butyl ammonium salicylate and choline.

The acidity of a salt that generates an acid weaker in acidity than the acid generated from an acid generating agent is indicated by the acid dissociation constant (pKa). The salt that generates an acid weaker in acidity than the acid generated from an acid generating agent is typically a salt with an acid dissociation constant of the acid generated from the salt being −3<pKa, preferably −1<pKa<7, and more preferably 0<pKa<5.

Examples of the salt that generates an acid weaker in acidity than the acid generated from an acid generating agent include the salts represented by the following formulas, the salt represented by formula (D) disclosed in Japanese Patent Laid-Open No. 2015-147926 (hereinafter referred to as “weak acid inner salt (D)” in some cases), and the salts disclosed in Japanese Patent Laid-Open No. 2012-229206, Japanese Patent Laid-Open No. 2012-6908, Japanese Patent Laid-Open No. 2012-72109, Japanese Patent Laid-Open No. 2011-39502 and Japanese Patent Laid-Open No. 2011-191745. The salt that generates an acid weaker in acidity than the acid generated from an acid generating agent is preferably a salt that generates a carboxylic acid weaker in acidity than the acid generated from an acid generating agent (a salt having a carboxylate anion), is more preferably a weak acid inner salt (D), and is further preferably a diphenyliodonium salt including a phenyl group substituted with a carboxylate anion among the weak acid inner salts (D).

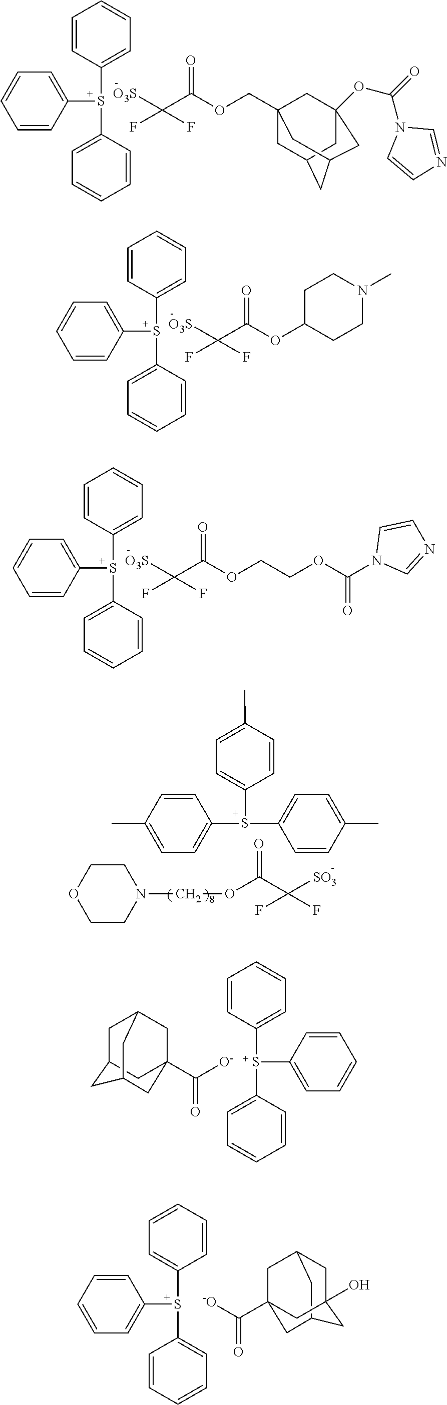

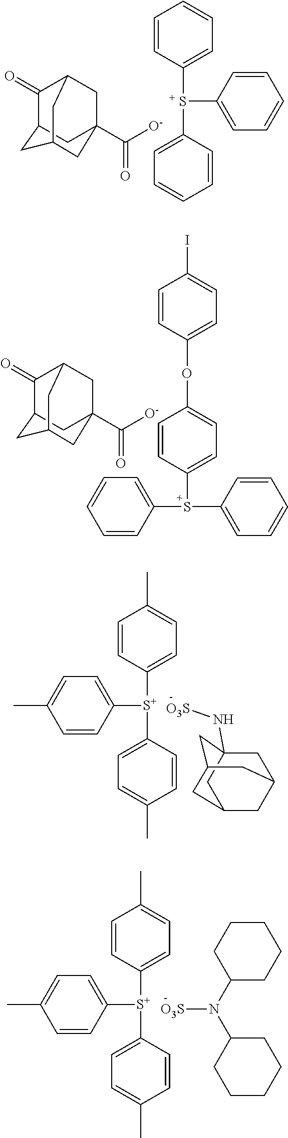

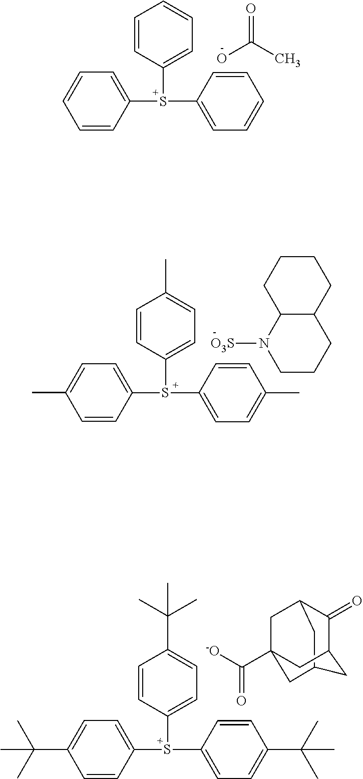

As the weak acid inner salt (D), a diphenyliodonium salt having an iodonium cation to which two phenyl groups are bonded and a carboxylate anion substituted for at least one of the two phenyl groups bonded to the iodonium cation is preferable, and specific examples thereof include a salt represented by the following formula:

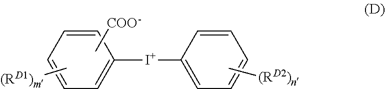

wherein
R^{D1} and R^{D2} each independently represent a hydrocarbon group having 1 to 12 carbon atoms, an alkoxy group having 1 to 6 carbon atoms, an acyl group having 2 to 7 carbon atoms, an acyloxy group having 2 to 7 carbon atoms, an alkoxycarbonyl group having 2 to 7 carbon atoms, a nitro group or a halogen atom; and
m′ and n′ each independently represent an integer of 0 to 4, and when m′ is 2 or larger, a plurality of R^{D1} may be the same or different from each other, and when n′ is 2 or larger, a plurality of R^{D2} may be the same or different from each other.

Examples of the hydrocarbon group of R^{D1} and R^{D2} include a chain hydrocarbon group, an alicyclic hydrocarbon group, an aromatic hydrocarbon group, and a group formed by combining these groups.

Examples of the chain hydrocarbon group include an alkyl group such as a methyl group, an ethyl group, a propyl group, an isopropyl group, a butyl group, an isobutyl group, a tert-butyl group, a pentyl group, a hexyl group and a nonyl group.

The alicyclic hydrocarbon group may be either monocyclic or polycyclic, and may be either saturated or unsaturated. Examples thereof include a cycloalkyl group such as a cyclopropyl group, a cyclobutyl group, a cyclopentyl group, a cyclohexyl group, a cyclononyl group and a cyclododecyl group, as well as a norbornyl group and an adamantyl group.

Examples of the aromatic hydrocarbon group include an aryl group such as a phenyl group, a 1-naphthyl group, a 2-naphthyl group, a 2-methylphenyl group, a 3-methylphenyl group, a 4-methylphenyl group, a 4-ethylphenyl group, a 4-propylphenyl group, a 4-isopropylphenyl group, a 4-butylphenyl group, a 4-t-butylphenyl group, a 4-hexylphenyl group, a 4-cyclohexylphenyl group, an anthryl group, a p-adamantylphenyl group, a tolyl group, a xylyl group, a cumenyl group, a mesityl group, a biphenyl group, a phenanthryl group, a 2,6-diethylphenyl group and a 2-methyl-6-ethylphenyl group.

Examples of the group formed by combining these groups include an alkyl-cycloalkyl group, a cycloalkyl-alkyl group and an aralkyl group (for example, a phenylmethyl group, a 1-phenylethyl group, a 2-phenylethyl group, a 1-phenyl-1-propyl group, a 1-phenyl-2-propyl group, a 2-phenyl-2-propyl group, a 3-phenyl-1-propyl group, a 4-phenyl-1-butyl group, a 5-phenyl-1-pentyl group and a 6-phenyl-1-hexyl group).

Examples of the alkoxy group include a methoxy group and an ethoxy group.

Examples of the acyl group include an acetyl group, a propanoyl group, a benzoyl group and a cyclohexanecarbonyl group.

Examples of the acyloxy group include the group in which an oxy group (—O—) is bonded to the above-described acyl group.

Examples of the alkoxycarbonyl group include a group in which a carbonyl group (—CO—) is bonded to the above-described alkoxy group.

Examples of the halogen atom include a fluorine atom, a chlorine atom and a bromine atom.

R^{D1} and R^{D2} are each independently preferably an alkyl group having 1 to 8 carbon atoms, a cycloalkyl group having 3 to 10 carbon atoms, an alkoxy group having 1 to 6 carbon atoms, an acyl group having 2 to 4 carbon atoms, an acyloxy group having 2 to 4 carbon atoms, an alkoxycarbonyl group having 2 to 4 carbon atoms, a nitro group or a halogen atom.

m′ and n′ are each independently preferably an integer of 0 to 2, and more preferably 0. When m′ is 2 or larger, a plurality of R^{D1} may be the same or different from each other, and when n′ is 2 or larger, a plurality of R^{D2} may be the same or different from each other.

More specific examples thereof include the following salts.

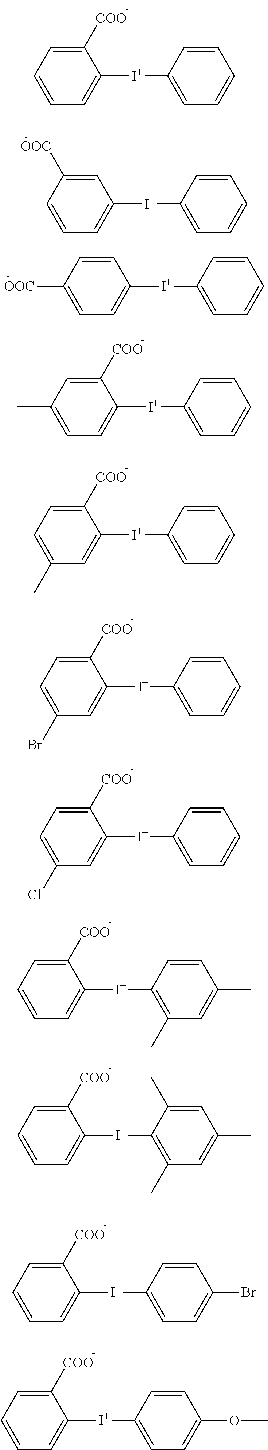

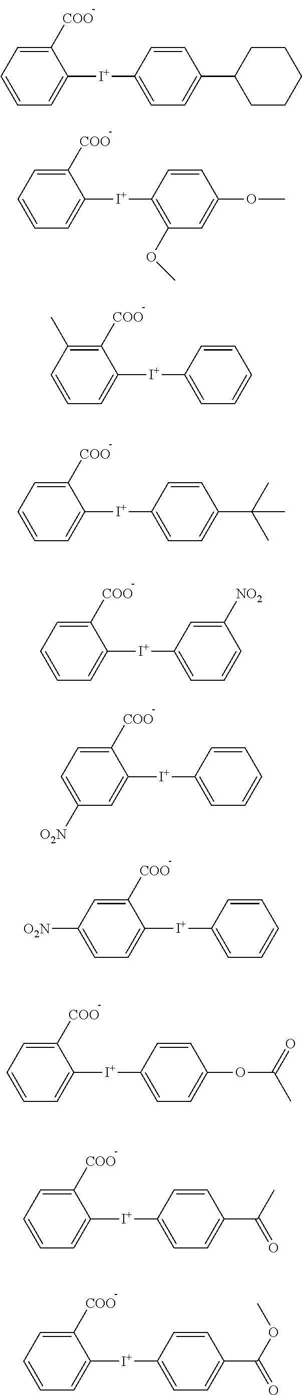

### <Other Components>

The resist composition of the present invention may contain components other than those described above (hereinafter referred to as “other components (F)” in some cases) if necessary. The other components (F) are not particularly limited, and additives known in the resist field, such as a sensitizer, a dissolution inhibitor, a surfactant, a stabilizer and a dye, can be used.

### <Preparation of Resist Composition>

The resist composition of the present invention can be prepared by mixing the salt (I) or the compound or resin including the structural unit (IP), and, if necessary, the resin (A) and/or the like, the compound (B), a resin other than the resin (A) and/or the like, the solvent (E), the quencher (C), and the other components (F). The order of mixing is arbitrary and is not particularly limited. The temperature during mixing can be selected from 10 to 40° C. depending on, for example, the type of resin and the like, the solubility of the resin and the like in the solvent (E). The mixing time can be selected from 0.5 to 24 hours depending on the mixing temperature. The mixing means is not particularly limited, and stirring and mixing can be used.

After mixing the components, it is preferable to filter the mixture using a filter with a pore size of about 0.003 to 0.2 μm.

### <Method for Producing Resist Pattern>

The method for producing a resist pattern of the present invention includes:
(1) a step of applying the resist composition of the present invention onto a substrate;
(2) a step of drying the applied resist composition to form a composition layer;
(3) a step of exposing the composition layer;
(4) a step of heating the exposed composition layer; and
(5) a step of developing the composition layer after heating.

The resist composition can be applied onto a substrate using a commonly used device such as a spin coater. Examples of the substrate include an inorganic substrate such as a silicon wafer, and an organic substrate with a resist film or the like formed on the surface thereof. Before applying the resist composition, the substrate may be cleaned, and an anti-reflective film or the like may be formed on the substrate.

The applied composition is dried to remove the solvent and form a composition layer. Drying is performed, for example, by evaporating the solvent using a heating device such as a hot plate (so-called pre-baking), or by using a vacuum device. The heating temperature is preferably 50 to 200° C., and the heating time is preferably 10 to 180 seconds. The pressure during vacuum drying is preferably about 1 to 1.0×10⁵ Pa.

The resulting composition layer is typically exposed using an exposure machine. The exposure machine may be an immersion exposure machine. Various types of exposure light sources can be used, including those that emit ultraviolet laser light such as KrF excimer laser (wavelength 248 nm), ArF excimer laser (wavelength 193 nm), and F₂ excimer laser (wavelength 157 nm), those that convert the wavelength of laser light from a solid-state laser light source (YAG or semiconductor laser) to emit harmonic laser light in the far ultraviolet or vacuum ultraviolet range, and those that irradiate with electron beams or extreme ultraviolet light (EUV). In the present specification, irradiation with these types of radiation may be collectively referred to as “exposure”. Exposure is typically performed through a mask corresponding to the desired pattern. If the exposure light source is an electron beam, exposure may be performed by direct drawing without using a mask.

The exposed composition layer is subjected to a heat treatment (so-called post-exposure bake) in order to promote the deprotection reaction of the acid labile groups. The heating temperature is typically about 50 to 200° C., preferably about 70 to 150° C. A chemical treatment (silylation) may be performed to adjust the hydrophilicity or hydrophobicity of the resin on the surface side of the heated composition. In addition, the steps of applying a resist composition, drying, exposing, and heating may be repeated on the exposed composition layer before development.

The heated composition layer is typically developed using a developing device with a developer. Examples of the development method include the dipping method, the paddle method, the spray method, and the dynamic dispensing method. The development temperature is preferably, for example, 5 to 60° C., and the development time is preferably, for example, 5 to 300 seconds. Selecting the type of developer as follows allows a positive resist pattern or a negative resist pattern to be produced.

When a positive resist pattern is produced from the resist composition of the present invention, an alkaline developer is used as the developer. The alkaline developer may be any of various alkaline aqueous solutions used in this field. Examples thereof include an aqueous solution of tetramethylammonium hydroxide and (2-hydroxyethyl)trimethyl ammonium hydroxide (commonly known as choline). The alkaline developer may include a surfactant.

After development, it is preferable to wash the resist pattern with ultrapure water, and then remove any water remaining on the substrate and pattern.

When a negative resist pattern is produced from the resist composition of the present invention, a developer including an organic solvent (hereinafter referred to as “organic developer” in some cases) is used as the developer.

Examples of the organic solvent included in the organic developer include: a ketone solvent such as 2-hexanone and 2-heptanone; a glycol ether ester solvent such as propylene glycol monomethyl ether acetate; an ester solvent such as butyl acetate; a glycol ether solvent such as propylene glycol monomethyl ether; an amide solvent such as N,N-dimethylacetamide; and an aromatic hydrocarbon solvent such as anisole.

The content ratio of the organic solvent in the organic developer is preferably 90% by mass or more and 100% by mass or less, more preferably 95% by mass or more and 100% by mass or less, and further preferably substantially only the organic solvent.

Among these, preferable organic developers are those including butyl acetate and/or 2-heptanone. The total content ratio of butyl acetate and 2-heptanone in the organic developer is preferably 50% by mass or more and 100% by mass or less, more preferably 90% by mass or more and 100% by mass or less, and further preferably substantially only butyl acetate and/or 2-heptanone.

The organic developer may include a surfactant. The organic developer may also include a small amount of water.

During development, development may be stopped by replacing the organic developer with a different type of solvent.

It is preferable to rinse the developed resist pattern with a rinse solution. The rinse solution is not particularly limited as long as it does not dissolve the resist pattern, and a solution including a general organic solvent can be used, preferably an alcohol solvent or an ester solvent.

After cleaning, it is preferable to remove the rinse solution remaining on the substrate and the pattern.

### <Applications>

The resist composition of the present invention is suitable as a resist composition for KrF excimer laser exposure, a resist composition for ArF excimer laser exposure, a resist composition for electron beam (EB) exposure, or a resist composition for EUV exposure, and is particularly suitable as a resist composition for electron beam (EB) exposure or a resist composition for EUV exposure, and is useful for semiconductor microfabrication.

### EXAMPLES

The present invention will be explained in more detail with reference to examples. In the examples, “%” and “parts” that indicate the content or amount used are based on mass unless otherwise specified.

The weight average molecular weight is a value determined by gel permeation chromatography. The analytical conditions for gel permeation chromatography are as follows.
Column: TSKgel Multipore HXL-M×3+guardcolumn (manufactured by Tosoh Corporation)
Eluent: tetrahydrofuran
Flow rate: 1.0 mL/min
Detector: RI detector
Column temperature: 40° C.
Injection volume: 100 μl
Molecular weight standard: Standard polystyrene (manufactured by Tosoh Corporation)

The structure of the compound was confirmed by measuring the molecular ion peak using mass spectrometry (LC: 1100 model manufactured by Agilent Technologies, Inc., MASS: LC/MSD model manufactured by Agilent Technologies, Inc.). In the following examples, the value of this molecular ion peak is indicated by “MASS”.

### Example 1: Synthesis of Salt Represented by Formula (I-1)

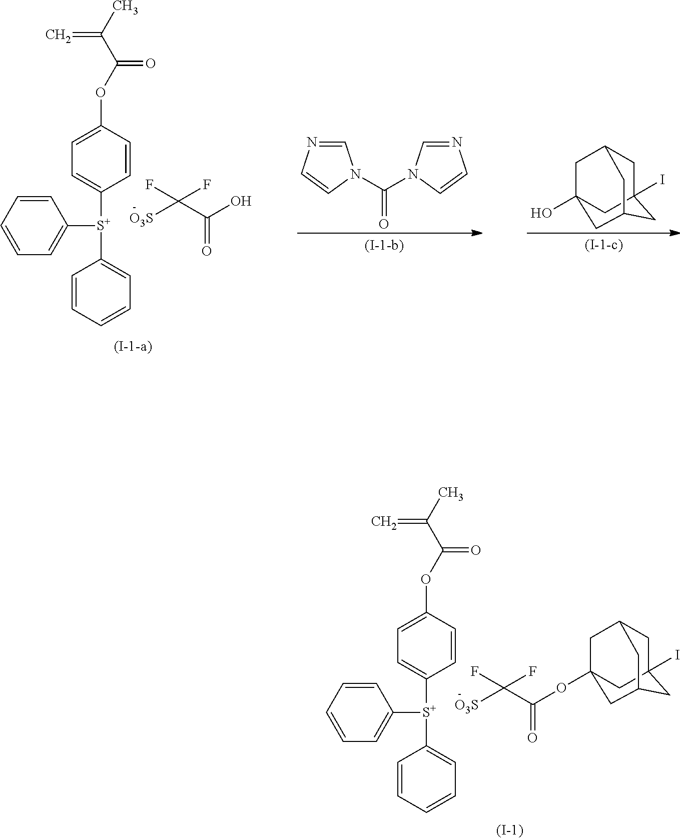

5.23 parts of the salt represented by formula (I-1-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 2.78 parts of the compound represented by formula (I-1-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 6.89 parts of the salt represented by formula (I-1).

MS (ESI (+) Spectrum): M⁺ 347.1

MS (ESI (−) Spectrum): M⁻ 435.0

### Example 2: Synthesis of Salt Represented by Formula (I-3)

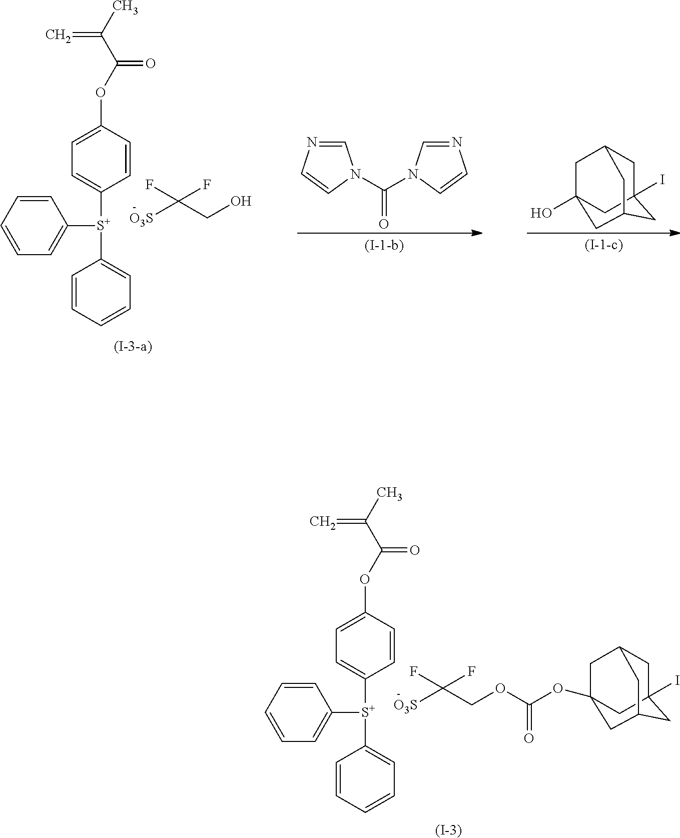

5.09 parts of the salt represented by formula (I-3-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 2.78 parts of the compound represented by formula (I-1-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 7.13 parts of the salt represented by formula (I-3).

MS (ESI (+) Spectrum): M⁺ 347.1

MS (ESI (−) Spectrum): M⁻ 465.0

### Example 3: Synthesis of Salt Represented by Formula (I-23)

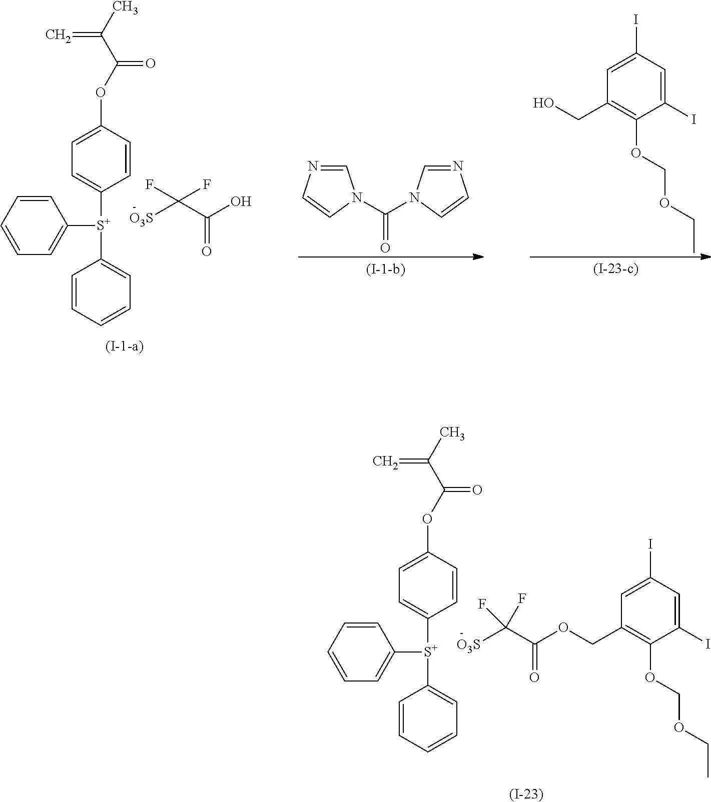

5.23 parts of the salt represented by formula (I-1-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.34 parts of the compound represented by formula (I-23-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.41 parts of the salt represented by formula (I-23).

MS (ESI (+) Spectrum): M⁺ 347.1

MS (ESI (−) Spectrum): M⁻ 590.8

### Example 4: Synthesis of Salt Represented by Formula (I-28)

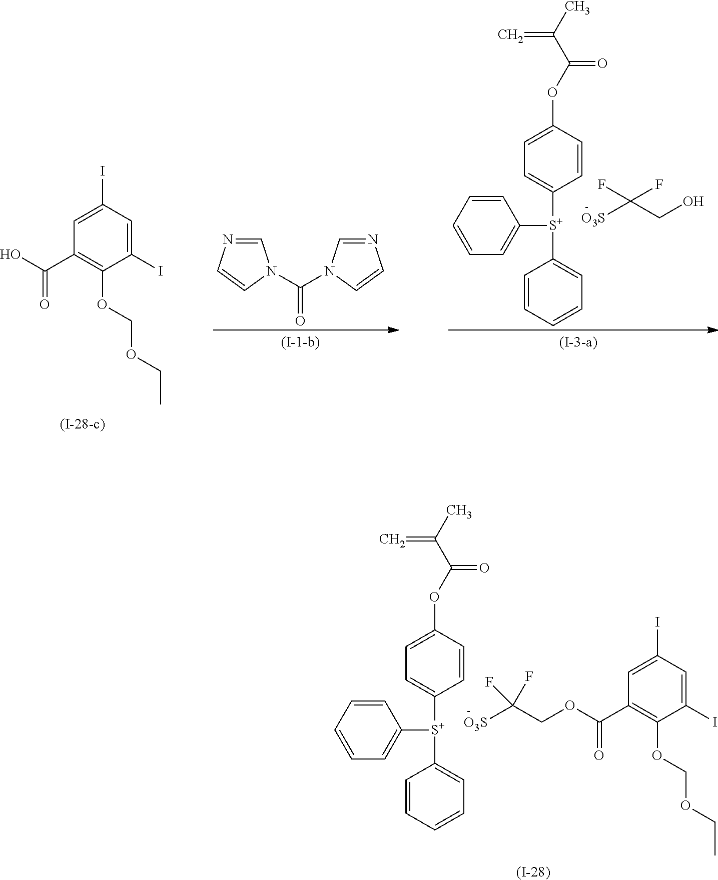

4.48 parts of the compound represented by formula (I-28-c) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 5.09 parts of the salt represented by formula (I-3-a) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.55 parts of the salt represented by formula (I-28).

MS (ESI (+) Spectrum): M⁺ 347.1

MS (ESI (−) Spectrum): M⁻ 590.8

### Example 5: Synthesis of Salt Represented by Formula (1-43

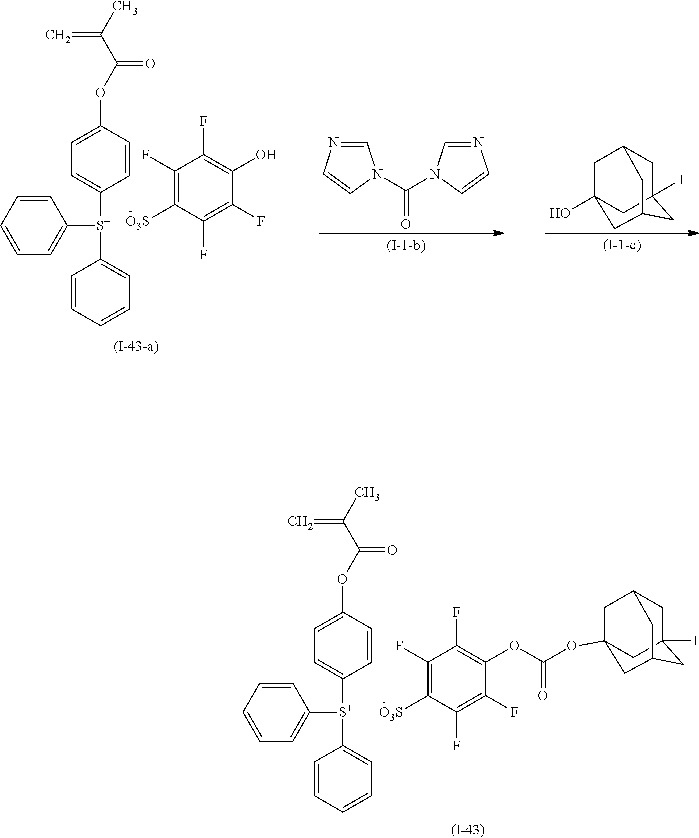

5.93 parts of the salt represented by formula (I-43-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 2.78 parts of the compound represented by formula (I-1-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 7.83 parts of the salt represented by formula (I-43).

MS (ESI (+) Spectrum): M⁺ 347.1

MS (ESI (−) Spectrum): M⁻ 549.0

### Example 6: Synthesis of Salt Represented by Formula (I-603)

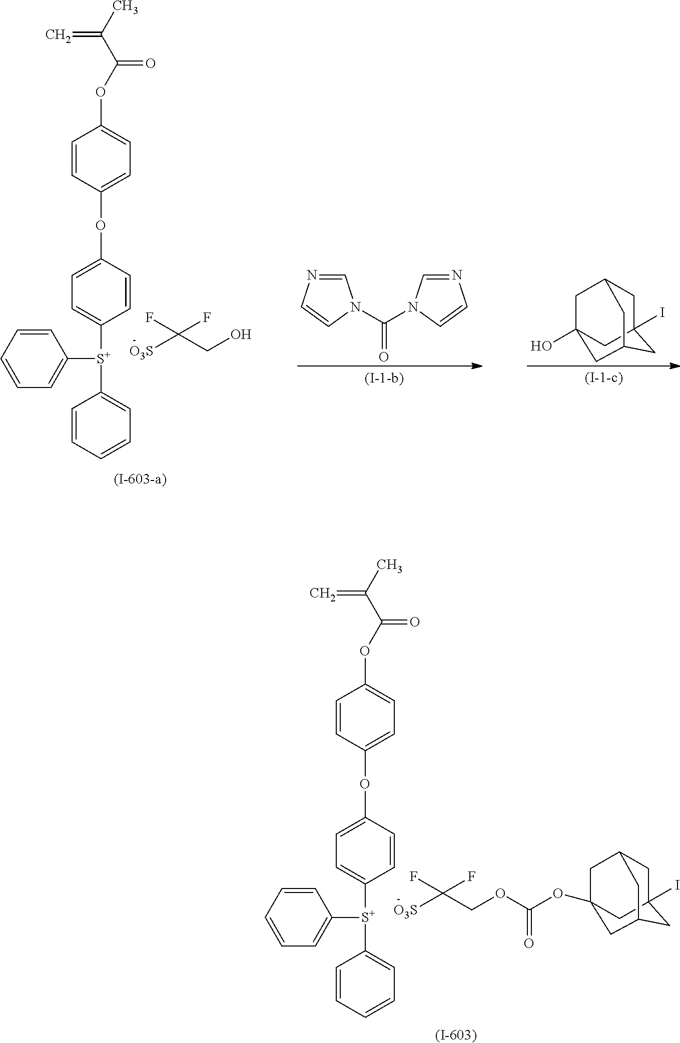

6.01 parts of the salt represented by formula (I-603-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 2.78 parts of the compound represented by formula (I-1-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.02 parts of the salt represented by formula (I-603).

MS (ESI (+) Spectrum): M⁺ 439.1

MS (ESI (−) Spectrum): M⁻ 465.0

### Example 7: Synthesis of Salt Represented by Formula (I-7)

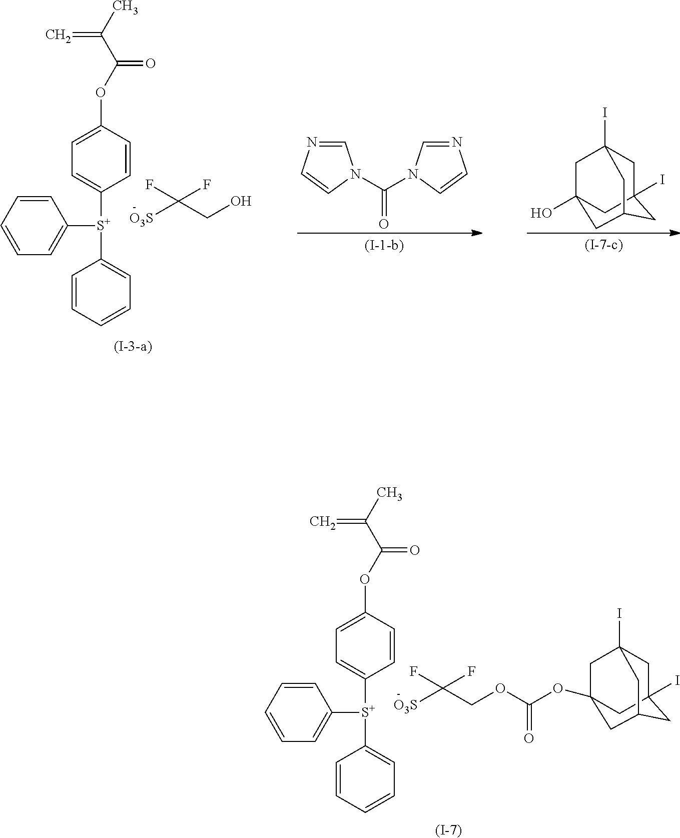

5.09 parts of the salt represented by formula (I-3-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.04 parts of the compound represented by formula (I-7-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.44 parts of the salt represented by formula (I-7).

MS (ESI (+) Spectrum): M⁺ 347.1

MS (ESI (−) Spectrum): M⁻ 590.9

### Example 8: Synthesis of Salt Represented by Formula (I-1107)

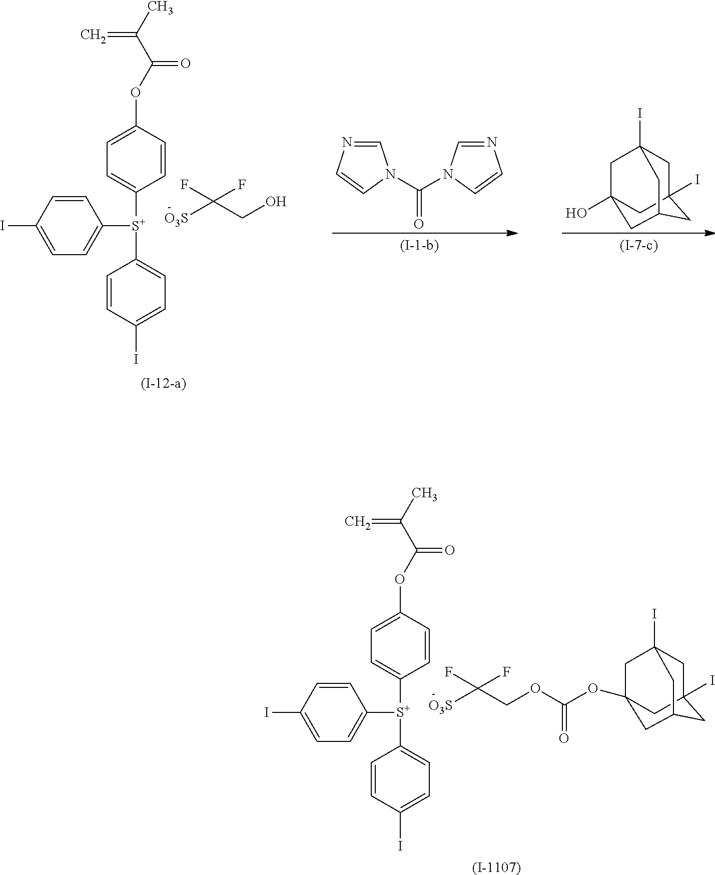

7.60 parts of the salt represented by formula (I-12-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.04 parts of the compound represented by formula (I-7-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 10.16 parts of the salt represented by formula (I-1107).

MS (ESI (+) Spectrum): M⁺ 598.9

MS (ESI (−) Spectrum): M⁻ 590.9

### Example 9: Synthesis of Salt Represented by Formula (I-4507)

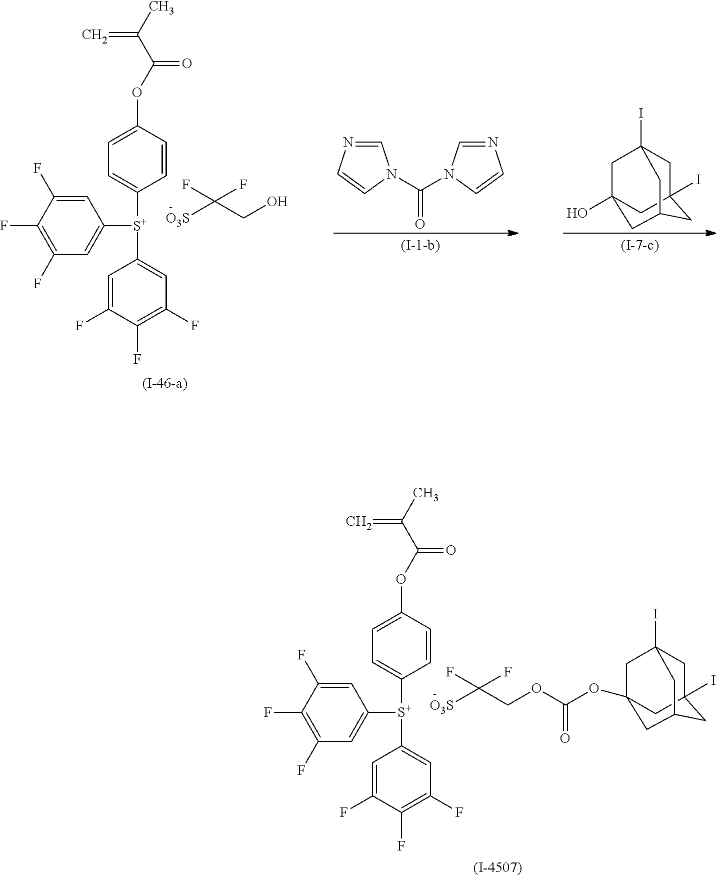

6.16 parts of the salt represented by formula (I-46-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.04 parts of the compound represented by formula (I-7-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 9.32 parts of the salt represented by formula (I-4507).

MS (ESI (+) Spectrum): M⁺ 455.1

MS (ESI (−) Spectrum): M⁻ 590.9

### Example 10: Synthesis of Salt Represented by Formula (I-10)

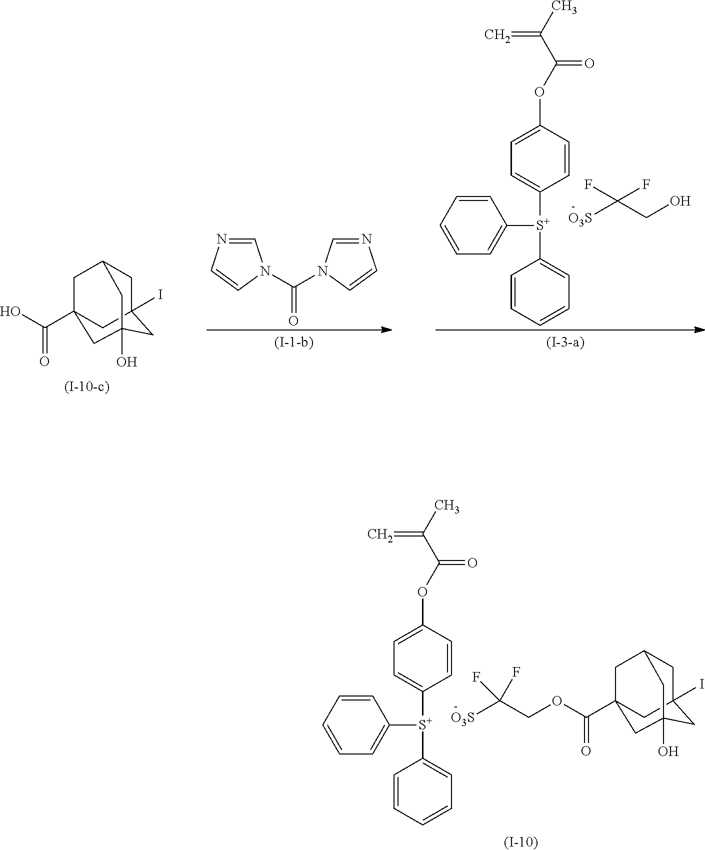

3.22 parts of the compound represented by formula (I-10-c) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 5.09 parts of the salt represented by formula (I-3-a) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.02 parts of the salt represented by formula (I-10).

MS (ESI (+) Spectrum): M⁺ 347.1

MS (ESI (−) Spectrum): M⁻ 465.0

### Example 11: Synthesis of Salt Represented by Formula (I-5001)

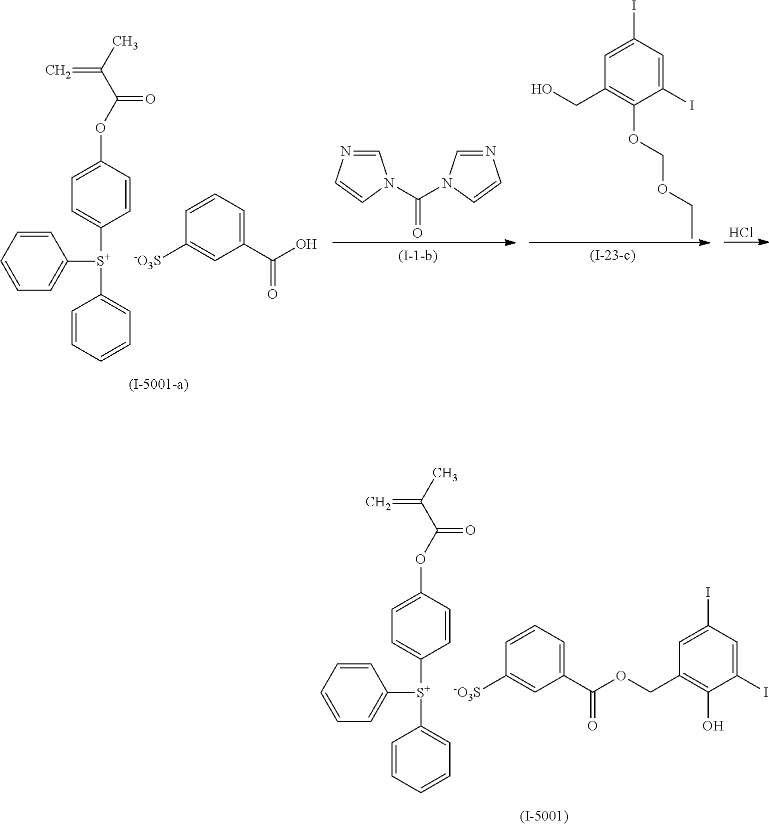

5.49 parts of the salt represented by formula (I-5001-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.34 parts of the compound represented by formula (I-23-c) was added to the resulting reaction mixture, which was stirred at 50° C. for 3 hours and then cooled to 23° C. 10 parts of 1 N hydrochloric acid was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 7.93 parts of the salt represented by formula (I-5001).

MS (ESI (+) Spectrum): M⁺ 347.1

MS (ESI (−) Spectrum): M⁻ 558.8

### Example 12: Synthesis of Salt Represented by Formula (I-5151)

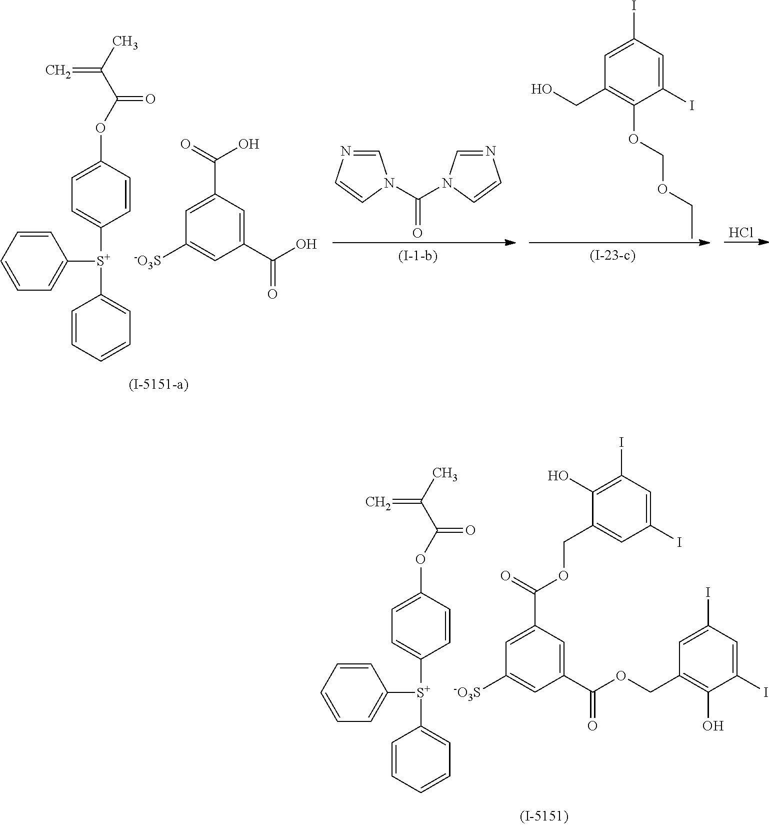

5.93 parts of the salt represented by formula (I-5151-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 8.68 parts of the compound represented by formula (I-23-c) was added to the resulting reaction mixture, which was stirred at 50° C. for 3 hours and then cooled to 23° C. 10 parts of 1 N hydrochloric acid was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 11.05 parts of the salt represented by formula (I-5151).

MS (ESI (+) Spectrum): M⁺ 347.1

MS (ESI (−) Spectrum): M⁻ 960.7

### Example 13: Synthesis of Salt Represented by Formula (I-5201)

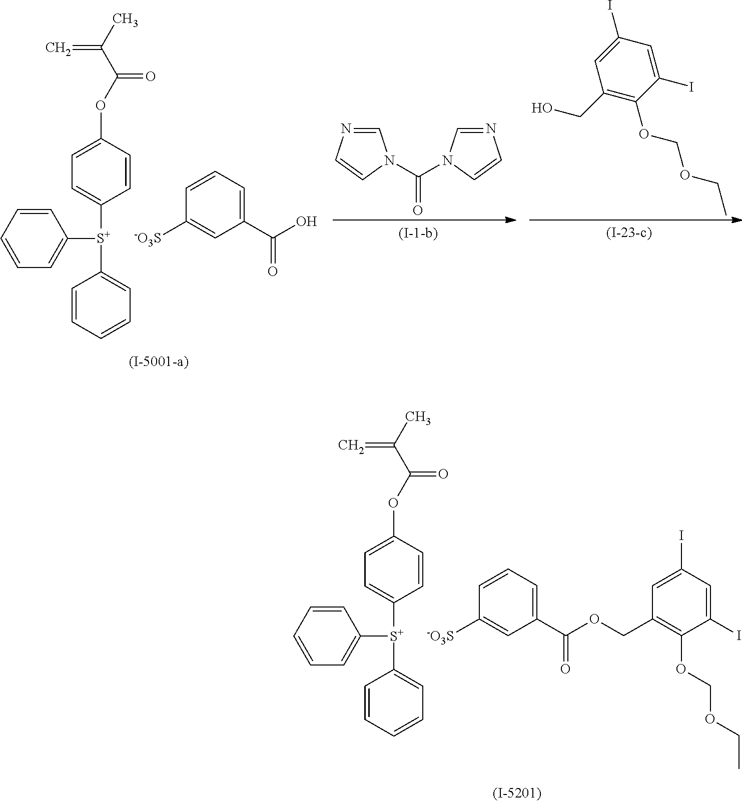

5.49 parts of the salt represented by formula (I-5001-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.34 parts of the compound represented by formula (I-23-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.44 parts of the salt represented by formula (I-5201).

MS (ESI (+) Spectrum): M⁺ 347.1

MS (ESI (−) Spectrum): M⁻ 616.9

### Example 14: Synthesis of Salt Represented by Formula (I-5301)

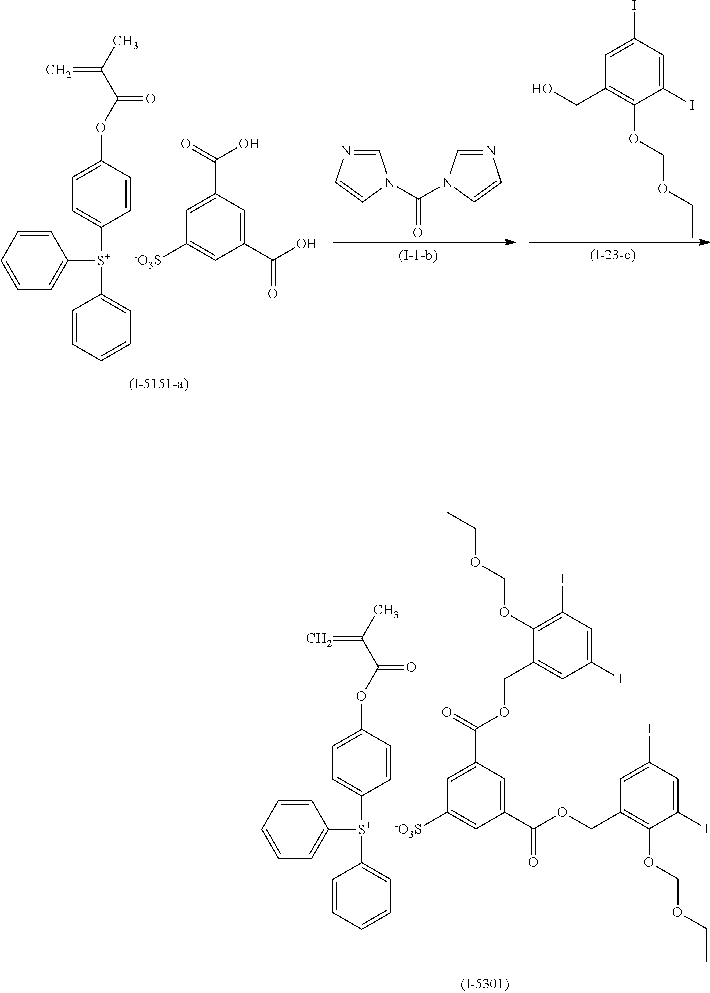

5.93 parts of the salt represented by formula (I-5151-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 8.68 parts of the compound represented by formula (I-23-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 12.18 parts of the salt represented by formula (I-5301).

MS (ESI (+) Spectrum): M⁺ 347.1

MS (ESI (−) Spectrum): M⁻ 1076.7

### Example 15: Synthesis of Salt Represented by Formula (I-5451)

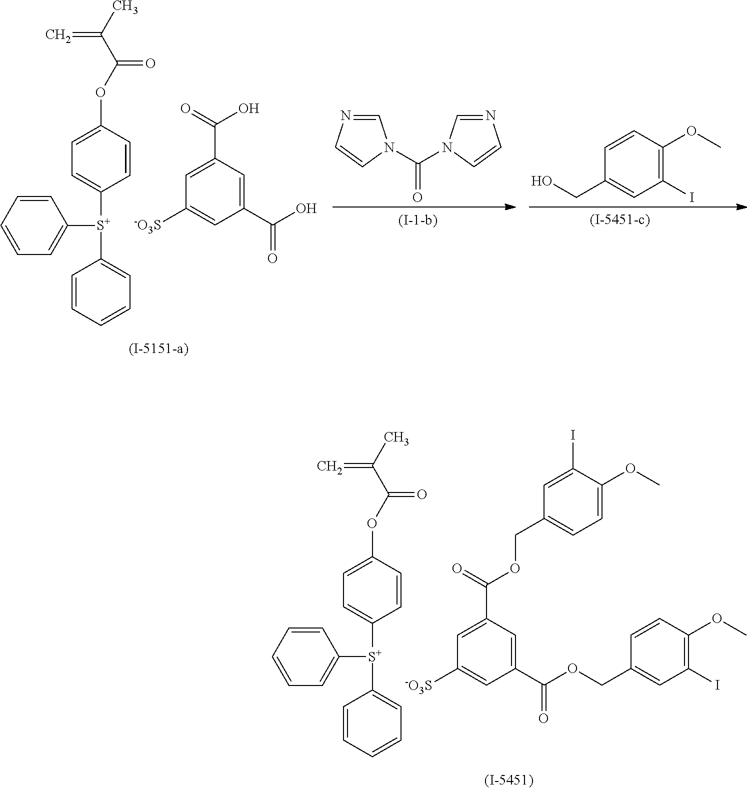

5.93 parts of the salt represented by formula (I-5151-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 5.28 parts of the compound represented by formula (I-5451-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 9.63 parts of the salt represented by formula (I-5451).

MS (ESI (+) Spectrum): M⁺ 347.1

MS (ESI (−) Spectrum): M⁻ 736.9

### Example 16: Synthesis of Salt Represented by Formula (I-5551)

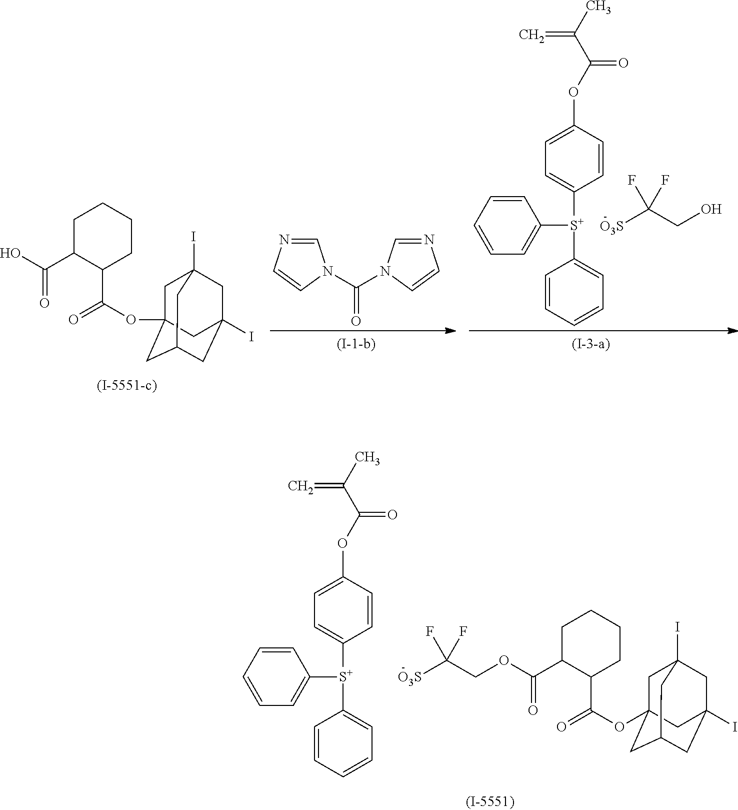

5.58 parts of the compound represented by formula (I-5551-c) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 5.09 parts of the salt represented by formula (I-3-a) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 9.11 parts of the salt represented by formula (I-5551).

MS (ESI (+) Spectrum): M⁺ 347.1

MS (ESI (−) Spectrum): M⁻ 700.9

### Example 17: Synthesis of Salt Represented by Formula (I-5601)

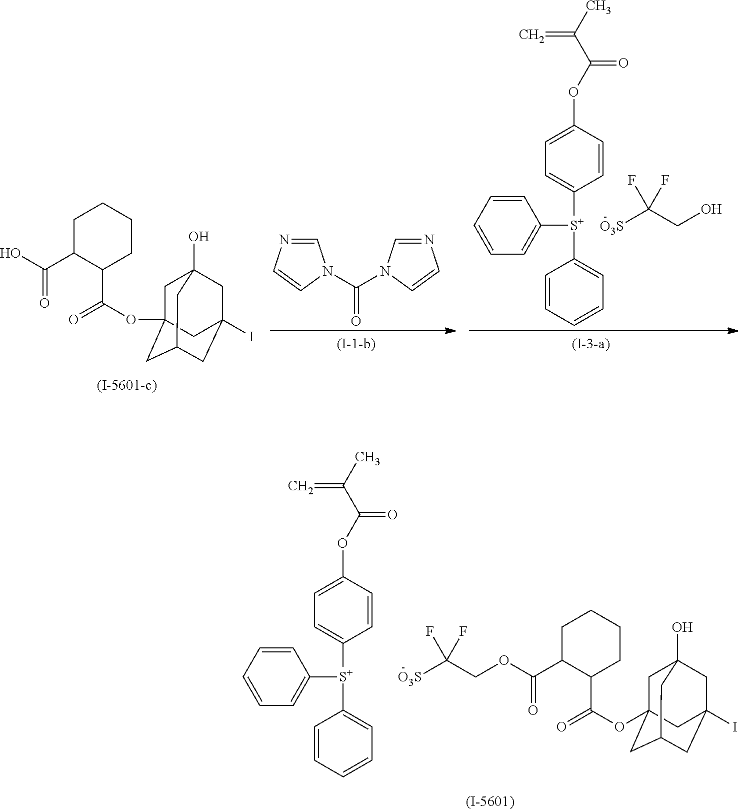

4.48 parts of the compound represented by formula (I-5601-c) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 5.09 parts of the salt represented by formula (I-3-a) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 7.49 parts of the salt represented by formula (I-5601).

MS (ESI (+) Spectrum): M⁺ 347.1

MS (ESI (−) Spectrum): M⁻ 591.0

### Example 18: Synthesis of Salt Represented by Formula (I-5701)

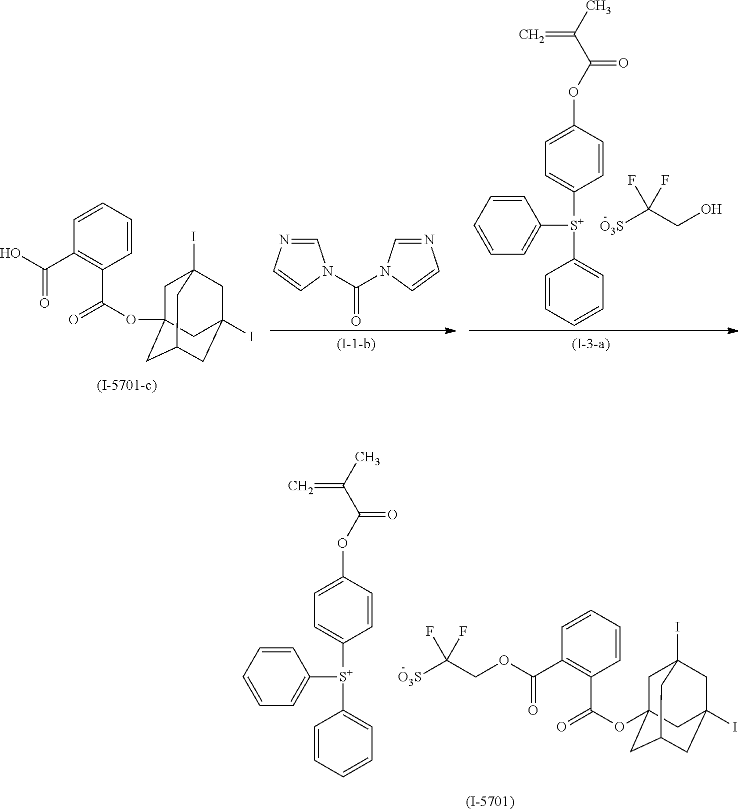

5.52 parts of the compound represented by formula (I-5701-c) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 5.09 parts of the salt represented by formula (I-3-a) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 9.05 parts of the salt represented by formula (I-5701).

MS (ESI (+) Spectrum): M⁺ 347.1

MS (ESI (−) Spectrum): M⁻ 694.9

### Example 19: Synthesis of Salt Represented by Formula (I-32)

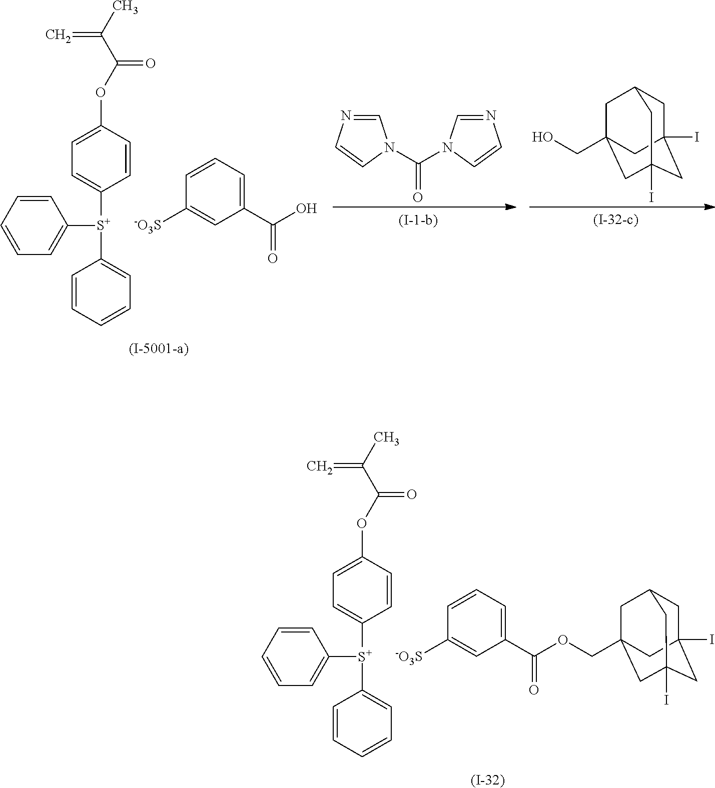

5.49 parts of the salt represented by formula (I-5001-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.18 parts of the compound represented by formula (I-32-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.22 parts of the salt represented by formula (I-32).

MS (ESI (+) Spectrum): M⁺ 347.1

MS (ESI (−) Spectrum): M⁻ 600.9

### Example 20: Synthesis of Salt Represented by Formula (I-40)

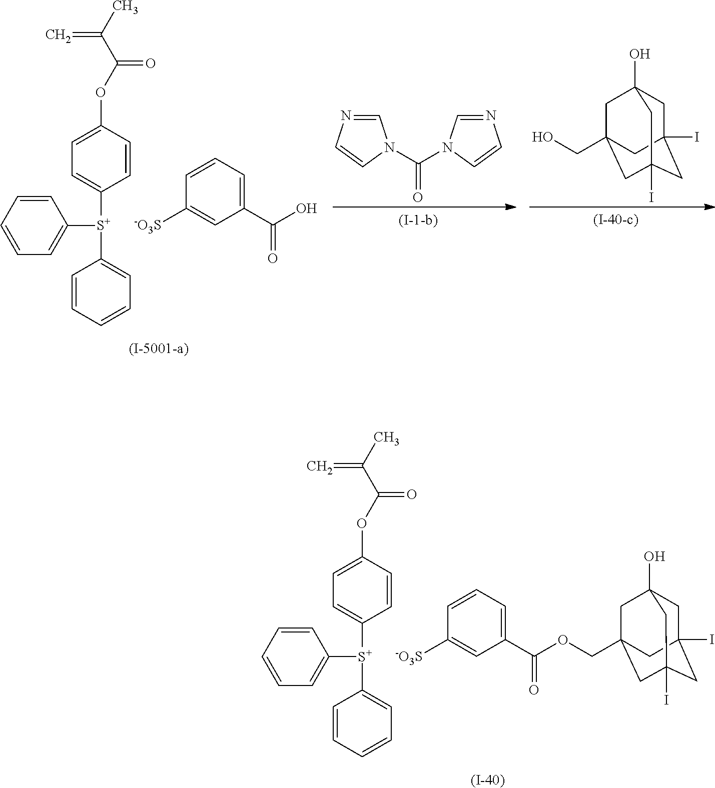

5.49 parts of the salt represented by formula (I-5001-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.34 parts of the compound represented by formula (I-40-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 8.01 parts of the salt represented by formula (I-40).

MS (ESI (+) Spectrum): M⁺ 347.1

MS (ESI (−) Spectrum): M⁻ 616.9

### Example 21: Synthesis of Salt Represented by Formula (I-6001)

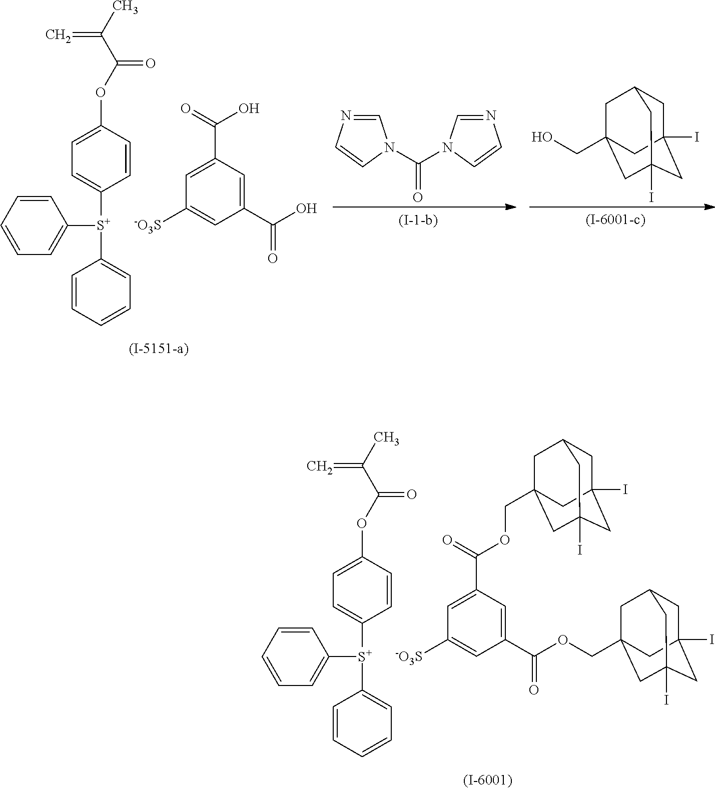

5.93 parts of the salt represented by formula (I-5151-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 8.36 parts of the compound represented by formula (I-6001-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 11.18 parts of the salt represented by formula (I-6001).

MS (ESI (+) Spectrum): M⁺ 347.1

MS (ESI (−) Spectrum): M⁻ 1044.8

### Example 22: Synthesis of Salt Represented by Formula (I-6051)

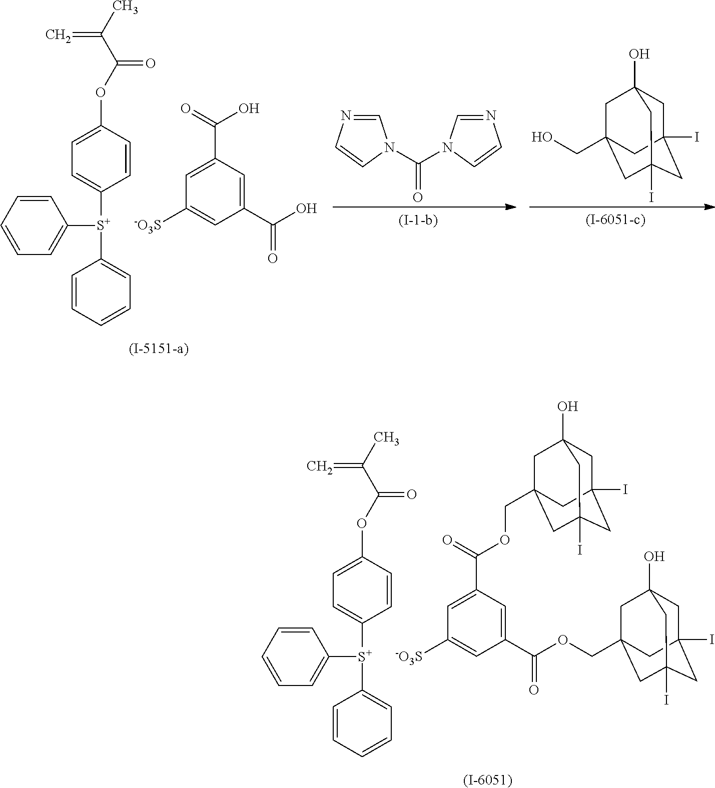

5.93 parts of the salt represented by formula (I-5151-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 8.68 parts of the compound represented by formula (I-6051-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 10.77 parts of the salt represented by formula (I-6051).

MS (ESI (+) Spectrum): M⁺ 347.1

MS (ESI (−) Spectrum): M⁻ 1076.8

### Example 23: Synthesis of Salt Represented by Formula (I-57)

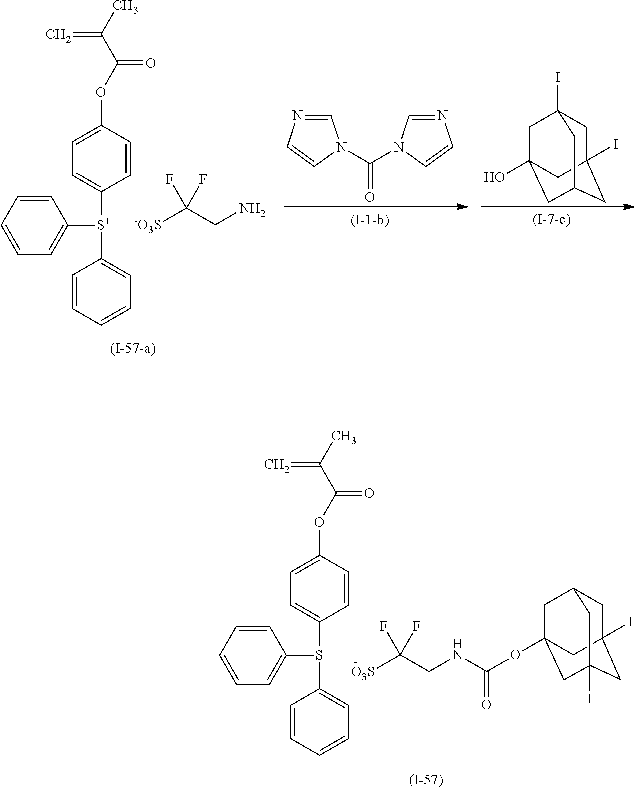

5.08 parts of the salt represented by formula (I-57-a) and 30 parts of chloroform were mixed and stirred at 23° C. for 30 minutes, 1.62 parts of the compound represented by formula (I-1-b) was then added, and after raising the temperature to 50° C., the mixture was stirred at 50° C. for 2 hours. 4.04 parts of the compound represented by formula (I-7-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 7.59 parts of the salt represented by formula (I-57).

MS (ESI (+) Spectrum): M⁺ 347.1

MS (ESI (−) Spectrum): M⁻ 589.9

### Example 24: Synthesis of Salt Represented by Formula (I-6061)

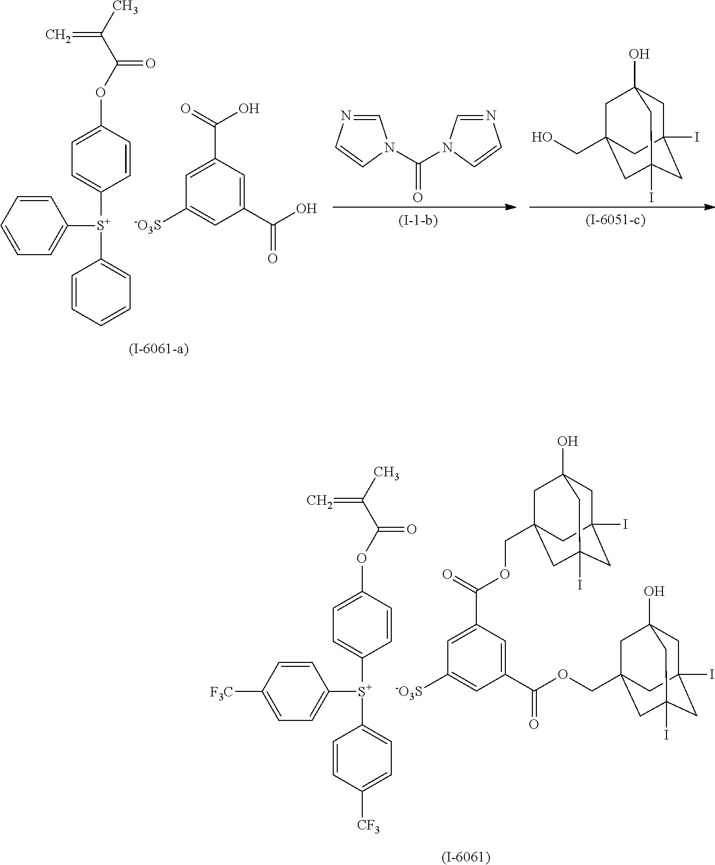

7.29 parts of the salt represented by formula (I-6061-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 8.68 parts of the compound represented by formula (I-6051-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 12.34 parts of the salt represented by formula (I-6061).

MS (ESI (+) Spectrum): M⁺ 483.1

MS (ESI (−) Spectrum): M⁻ 1076.8

### Example 25: Synthesis of Salt Represented by Formula (I-6099)

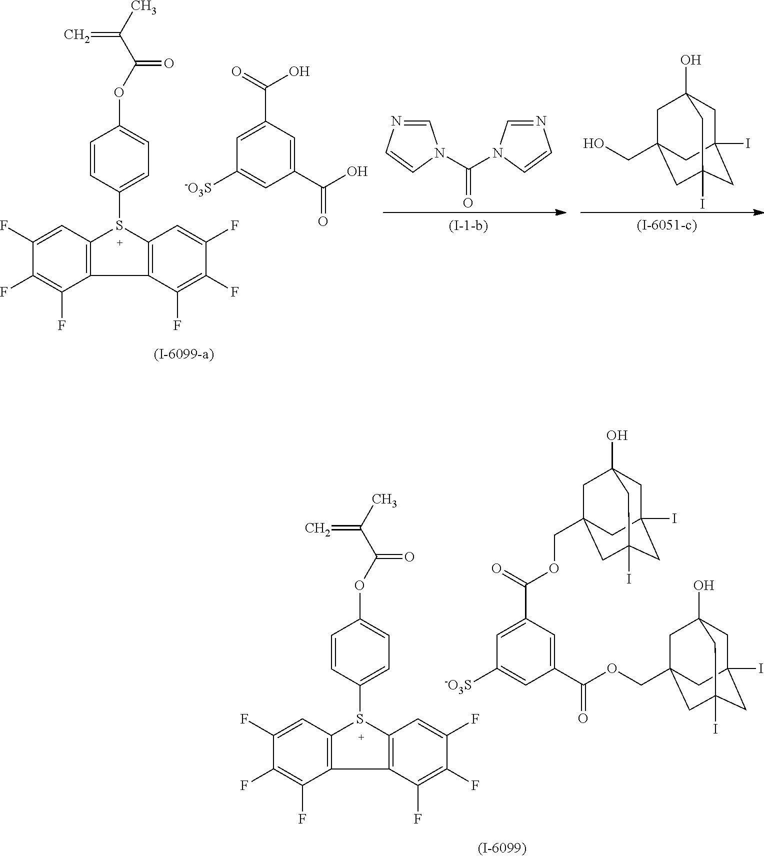

6.99 parts of the salt represented by formula (I-6099-a) and 30 parts of chloroform were mixed and stirred at 23° C. for 30 minutes, 3.24 parts of the compound represented by formula (I-1-b) was then added, and after raising the temperature to 50° C., the mixture was stirred at 50° C. for 2 hours. 8.68 parts of the compound represented by formula (I-6051-c) was added to the resulting reaction mixture, which was stirred at 50° C. for 3 hours and then cooled to 23° C. 10 parts of a 5% aqueous oxalic acid solution was added to the resulting mixture, which was stirred at 23° C. for 30 minutes, and then the organic layer was separated and taken out. 10 parts of ion exchanged water was added to the resulting organic layer, which was stirred at 23° C. for 30 minutes, and then the organic layer was separated and taken out. This water washing procedure was repeated five times. The resulting organic layer was concentrated, and 30 parts of tert-butyl methyl ether was added to the concentrated residue, which was stirred at 23° C. for 30 minutes. The supernatant was then removed, and the mixture was concentrated to provide 12.34 parts of the salt represented by formula (I-6099).

MS (ESI (+) Spectrum): M⁺ 453.0

MS (ESI (−) Spectrum): M⁻ 1076.8

### Synthesis of Resin

The compounds (monomers) used in the synthesis of the resin (A) are shown below. Hereinafter, these compounds will be referred to as “monomer (a1-2-6)” and the like according to the formula number thereof.

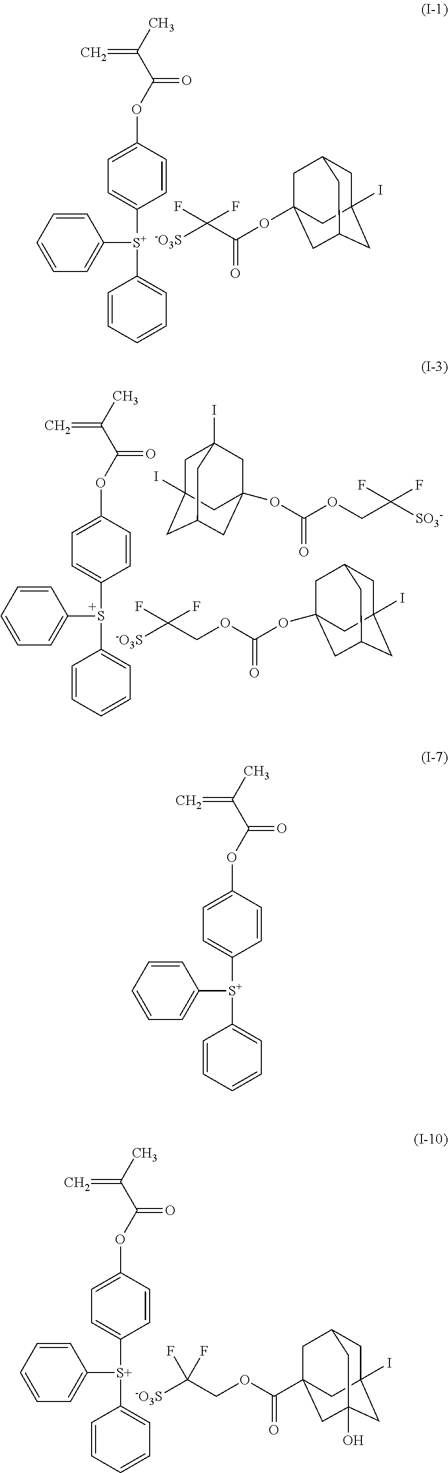

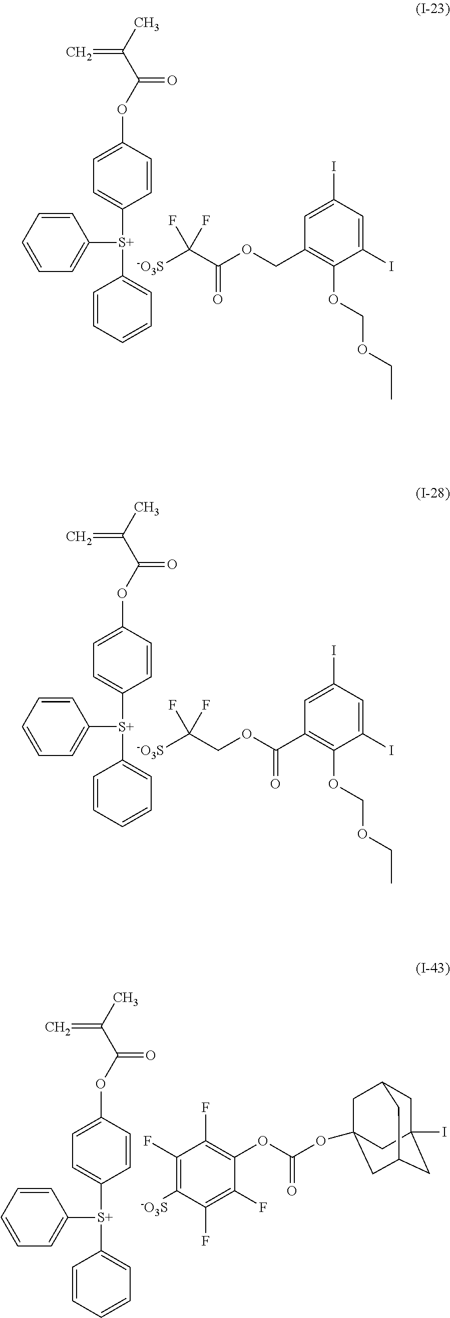

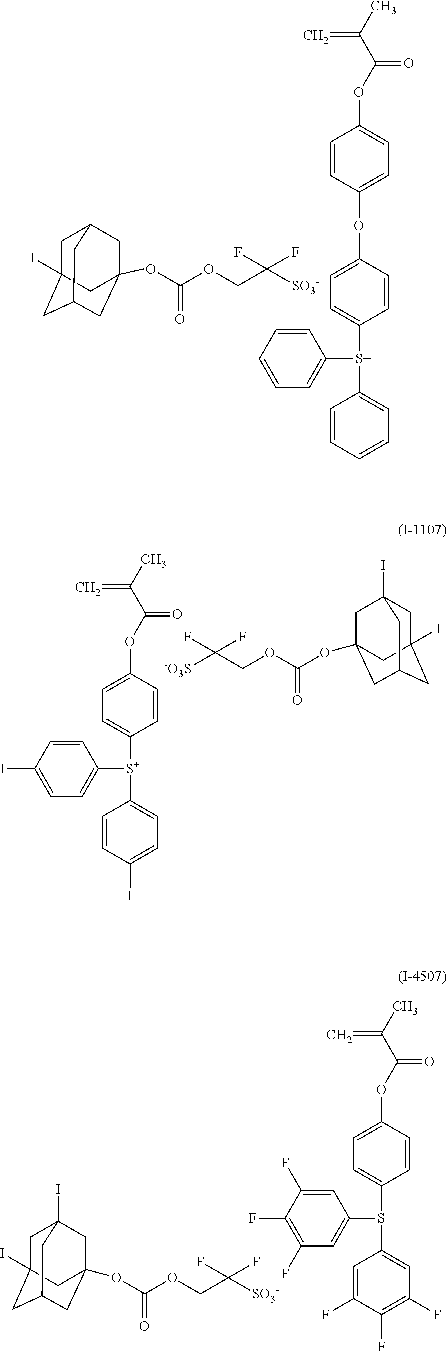

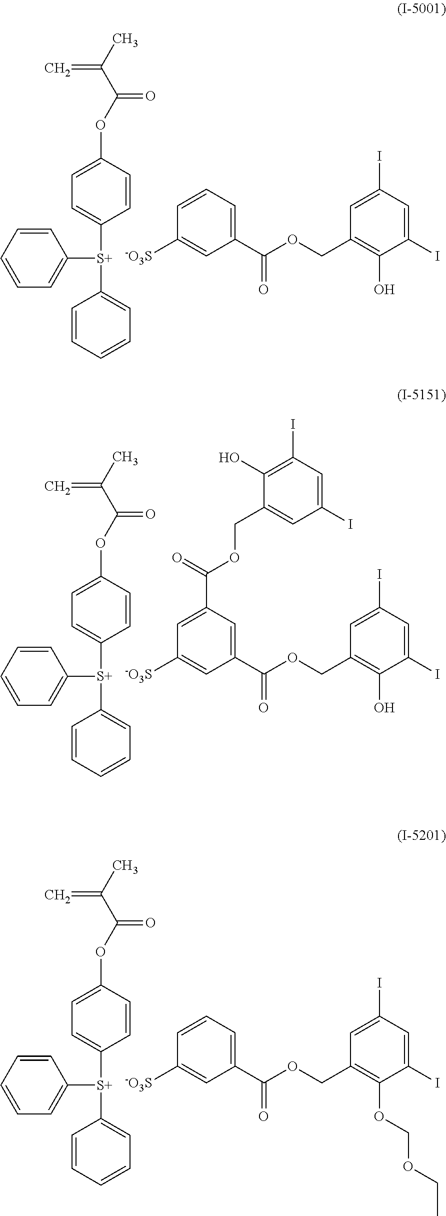

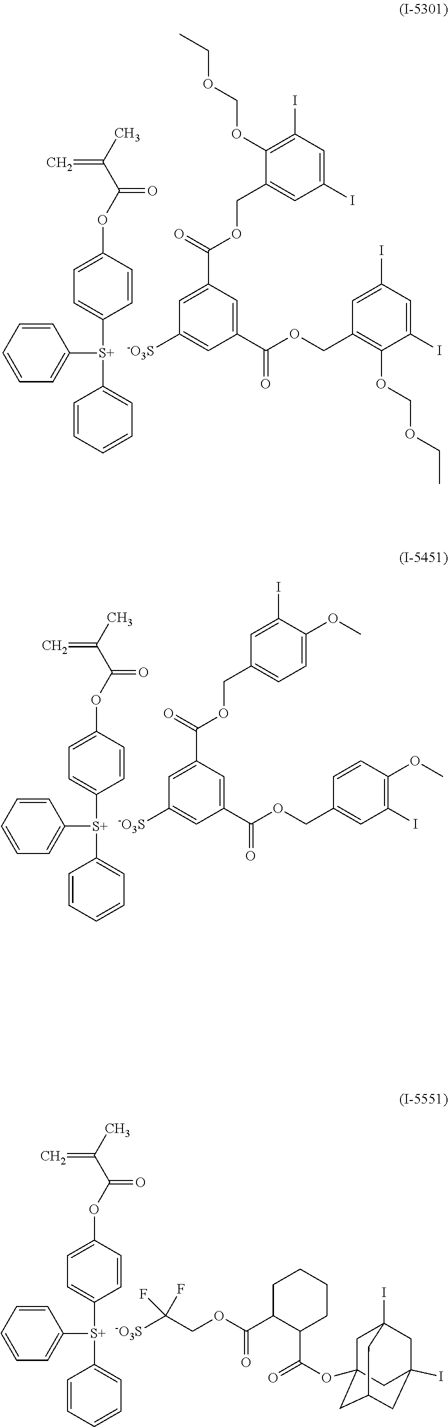

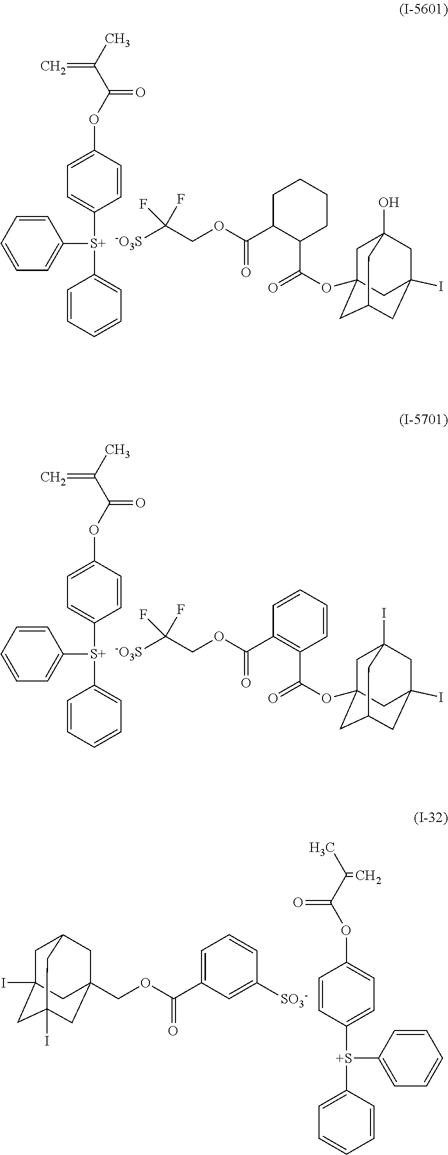

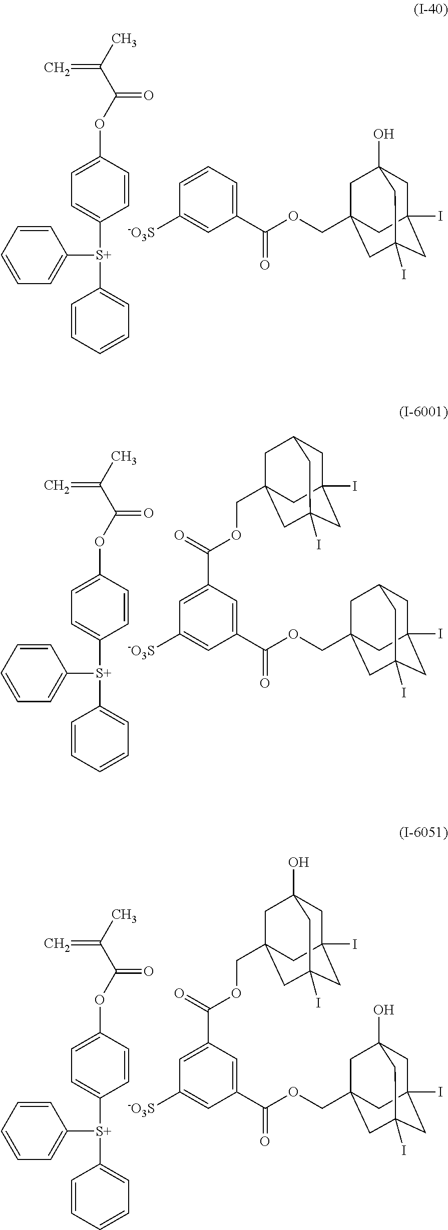

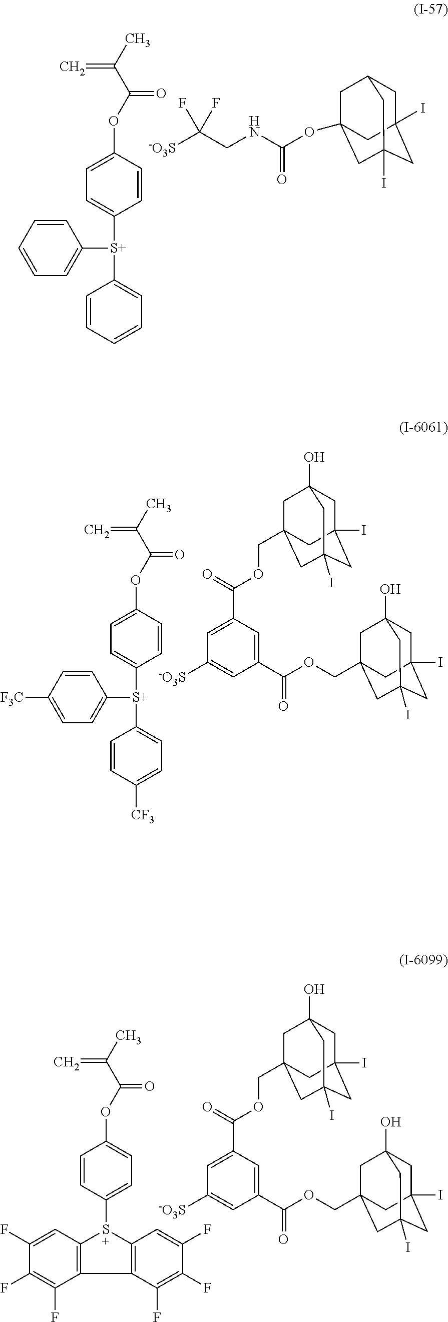

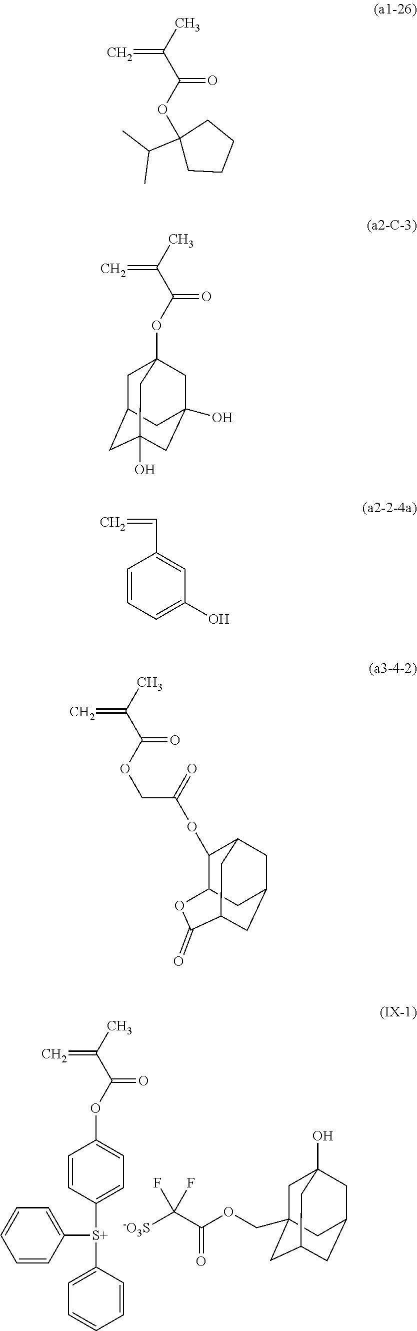

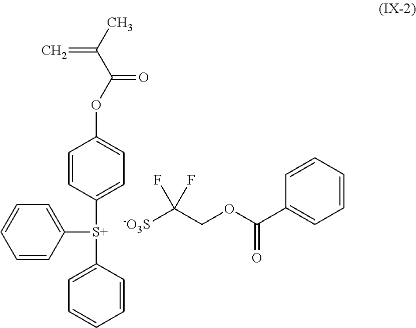

### Example 26 [Synthesis of Resin A1]

The monomer (a1-2-6), the monomer (a2-C-3), the monomer (a3-4-2), the monomer (a2-2-4a) and the monomer (I-1) were used as monomers and mixed in a molar ratio [monomer (a1-2-6):monomer (a2-C-3):monomer (a3-4-2):monomer (a2-2-4a):monomer (I-1)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A1 with a weight average molecular weight of about 5.6×10³ in a yield of 81%. This resin A1 has the following structural units.

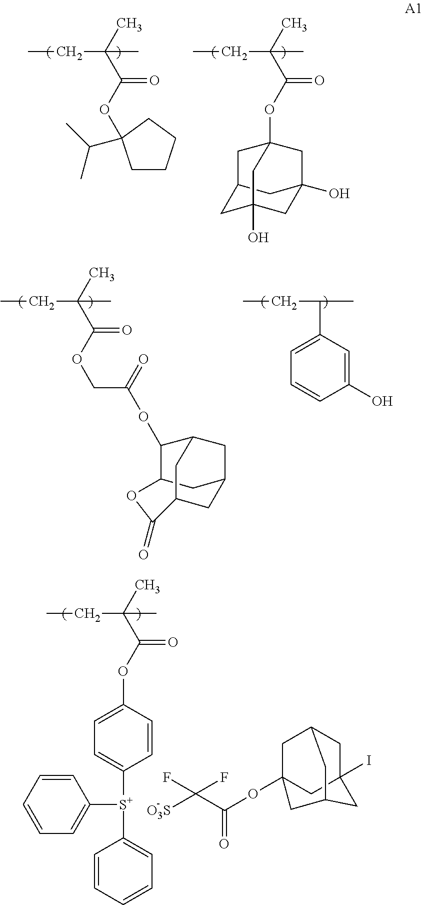

### Example 27 [Synthesis of Resin A2]

The monomer (a1-2-6), the monomer (a2-C-3), the monomer (a3-4-2), the monomer (a2-2-4a) and the monomer (I-3) were used as monomers and mixed in a molar ratio [monomer (a1-2-6):monomer (a2-C-3):monomer (a3-4-2):monomer (a2-2-4a):monomer (I-3)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A2 with a weight average molecular weight of about 5.6×10³ in a yield of 83%. This resin A2 has the following structural units.

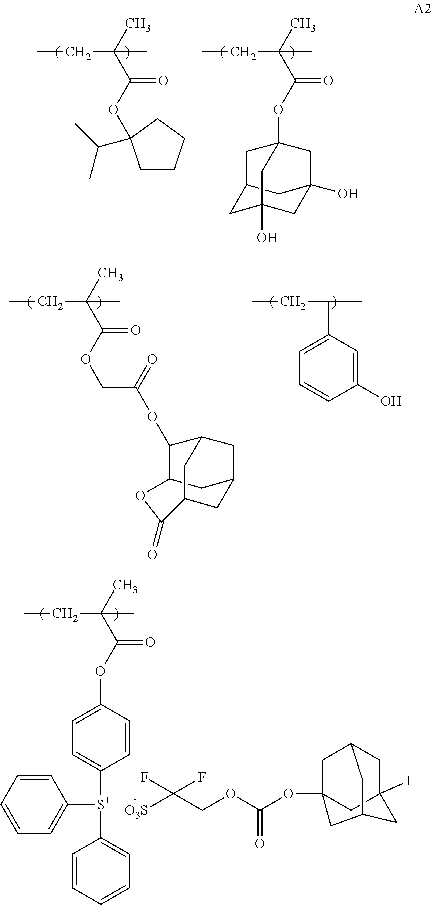

### Example 28 [Synthesis of Resin A3]

The monomer (a1-2-6), the monomer (a2-C-3), the monomer (a3-4-2), the monomer (a2-2-4a) and the monomer (I-23) were used as monomers and mixed in a molar ratio [monomer (a1-2-6):monomer (a2-C-3):monomer (a3-4-2):monomer (a2-2-4a):monomer (I-23)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A3 with a weight average molecular weight of about 5.4×10³ in a yield of 85%. This resin A3 has the following structural units.

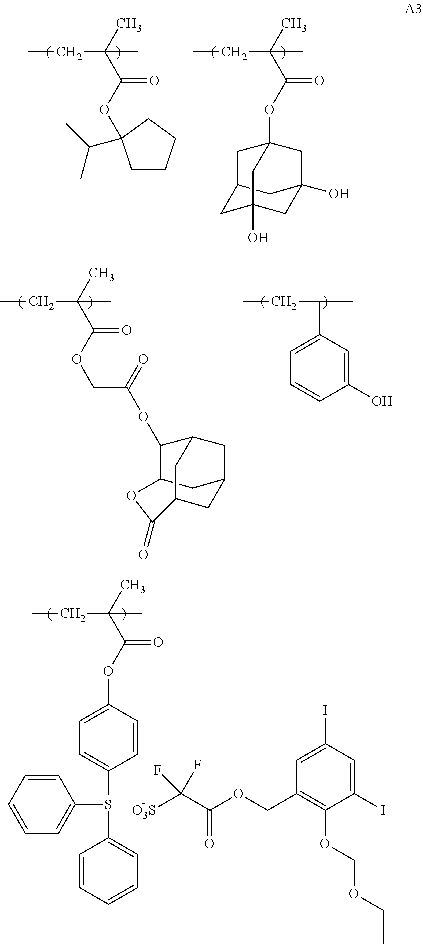

### Example 29 [Synthesis of Resin A4]

The monomer (a1-2-6), the monomer (a2-C-3), the monomer (a3-4-2), the monomer (a2-2-4a) and the monomer (I-28) were used as monomers and mixed in a molar ratio [monomer (a1-2-6):monomer (a2-C-3):monomer (a3-4-2):monomer (a2-2-4a):monomer (I-28)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A4 with a weight average molecular weight of about 5.2×10³ in a yield of 86%. This resin A4 has the following structural units.

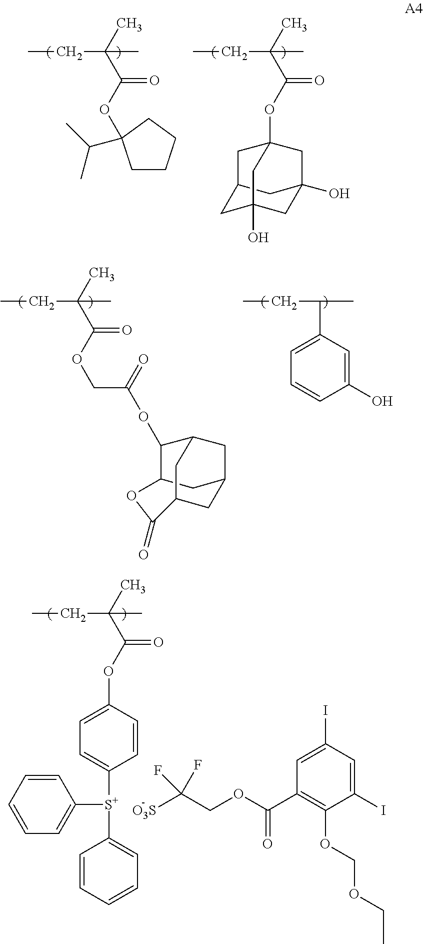

### Example 30 [Synthesis of Resin A5]

The monomer (a1-2-6), the monomer (a2-C-3), the monomer (a3-4-2), the monomer (a2-2-4a) and the monomer (I-43) were used as monomers and mixed in a molar ratio [monomer (a1-2-6):monomer (a2-C-3):monomer (a3-4-2):monomer (a2-2-4a):monomer (I-43)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A5 with a weight average molecular weight of about 5.5×10³ in a yield of 76%. This resin A5 has the following structural units.

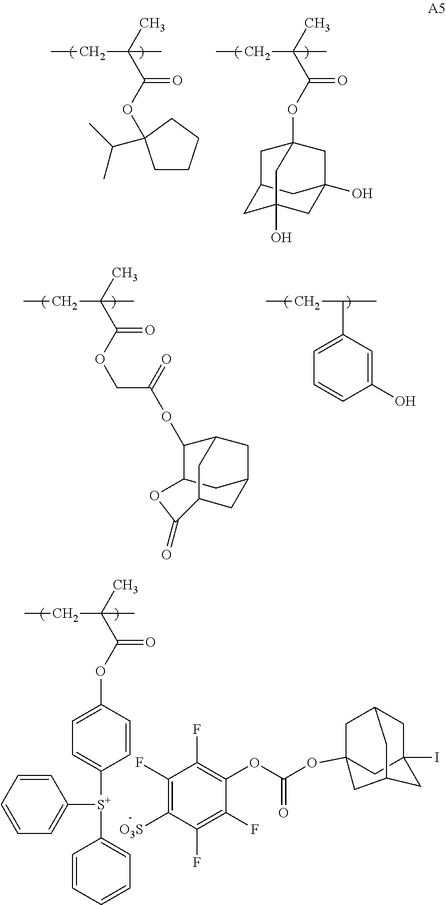

### Example 31 [Synthesis of Resin A6]

The monomer (a1-2-6), the monomer (a2-C-3), the monomer (a3-4-2), the monomer (a2-2-4a) and the monomer (I-603) were used as monomers and mixed in a molar ratio [monomer (a1-2-6):monomer (a2-C-3):monomer (a3-4-2):monomer (a2-2-4a):monomer (I-603)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A6 with a weight average molecular weight of about 5.7×10³ in a yield of 79%. This resin A6 has the following structural units.

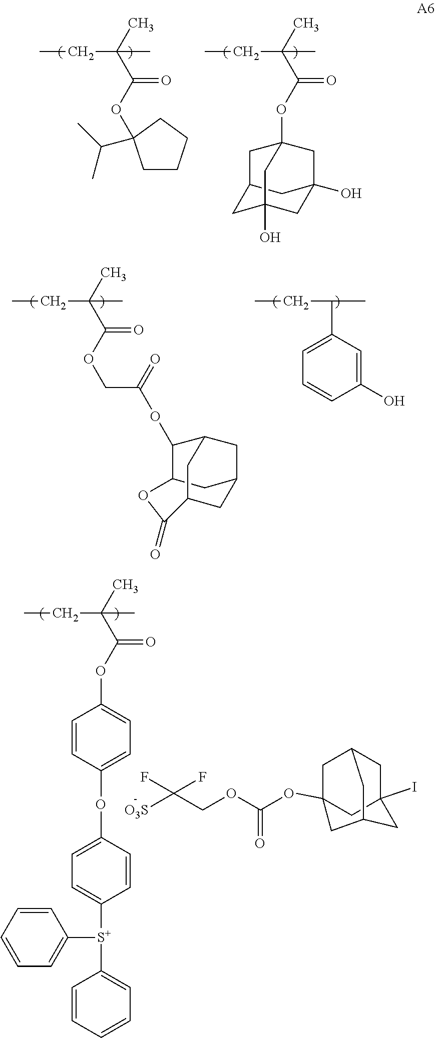

### Example 32 [Synthesis of Resin A7]

The monomer (a1-2-6), the monomer (a2-C-3), the monomer (a3-4-2), the monomer (a2-2-4a) and the monomer (I-7) were used as monomers and mixed in a molar ratio [monomer (a1-2-6):monomer (a2-C-3):monomer (a3-4-2):monomer (a2-2-4a):monomer (I-7)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A7 with a weight average molecular weight of about 5.4×10³ in a yield of 77%. This resin A7 has the following structural units.

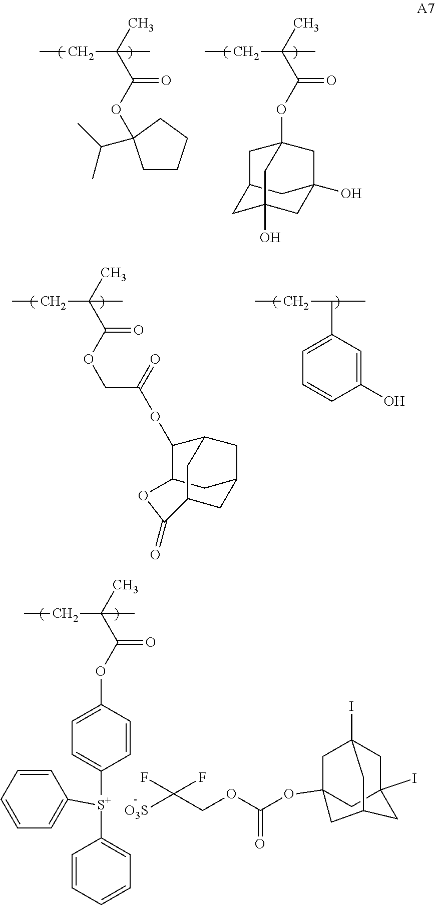

### Example 33 [Synthesis of Resin A8]

The monomer (a1-2-6), the monomer (a2-C-3), the monomer (a3-4-2), the monomer (a2-2-4a) and the monomer (I-1107) were used as monomers and mixed in a molar ratio [monomer (a1-2-6):monomer (a2-C-3):monomer (a3-4-2):monomer (a2-2-4a):monomer (I-1107)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A8 with a weight average molecular weight of about 5.6×10³ in a yield of 70%. This resin A8 has the following structural units.

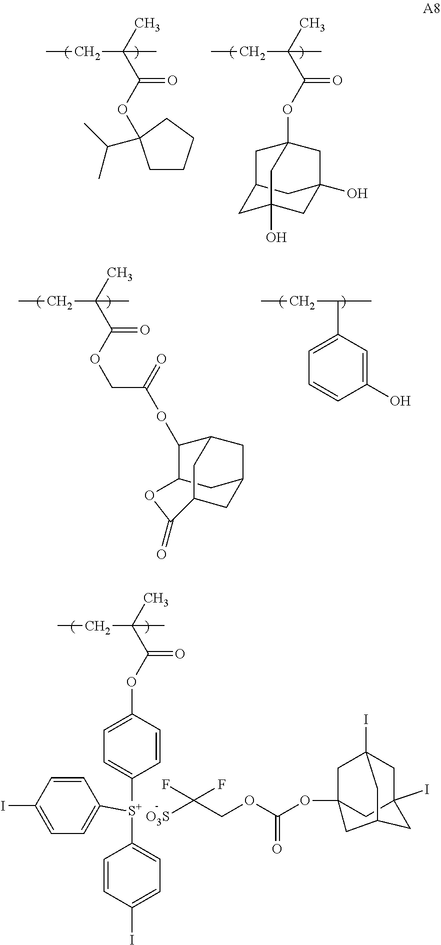

### Example 34 [Synthesis of Resin A9]

The monomer (a1-2-6), the monomer (a2-C-3), the monomer (a3-4-2), the monomer (a2-2-4a) and the monomer (I-4507) were used as monomers and mixed in a molar ratio [monomer (a1-2-6):monomer (a2-C-3):monomer (a3-4-2):monomer (a2-2-4a):monomer (I-4507)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A9 with a weight average molecular weight of about 5.6×10³ in a yield of 78%. This resin A9 has the following structural units.

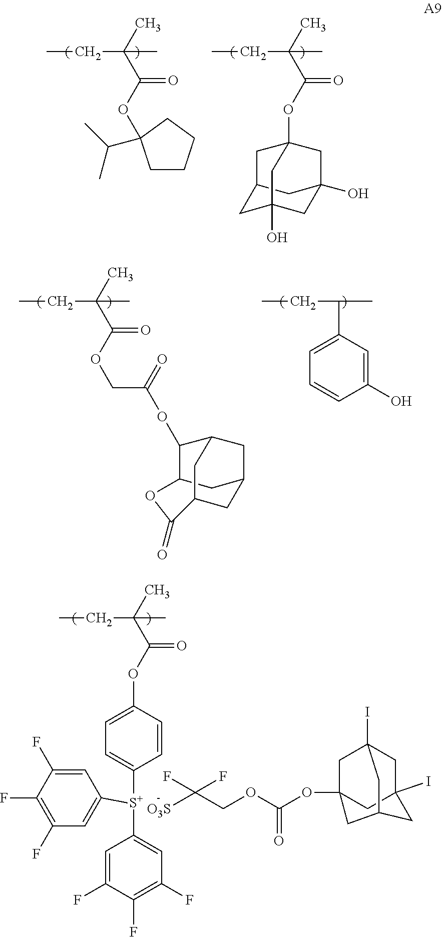

### Example 35 [Synthesis of Resin A10]

The monomer (a1-2-6), the monomer (a2-C-3), the monomer (a3-4-2), the monomer (a2-2-4a) and the monomer (I-10) were used as monomers and mixed in a molar ratio [monomer (a1-2-6):monomer (a2-C-3):monomer (a3-4-2):monomer (a2-2-4a):monomer (I-10)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A10 with a weight average molecular weight of about 5.5×10³ in a yield of 77%. This resin A10 has the following structural units.

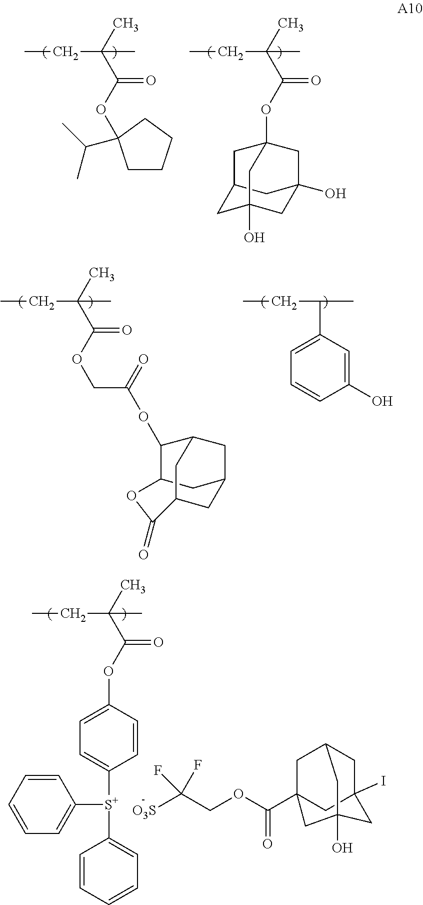

### Example 36 [Synthesis of Resin A11]

The monomer (a1-2-6), the monomer (a2-C-3), the monomer (a3-4-2), the monomer (a2-2-4a) and the monomer (I-5001) were used as monomers and mixed in a molar ratio [monomer (a1-2-6):monomer (a2-C-3):monomer (a3-4-2):monomer (a2-2-4a):monomer (I-5001)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A11 with a weight average molecular weight of about 5.4×10³ in a yield of 83%. This resin A11 has the following structural units.

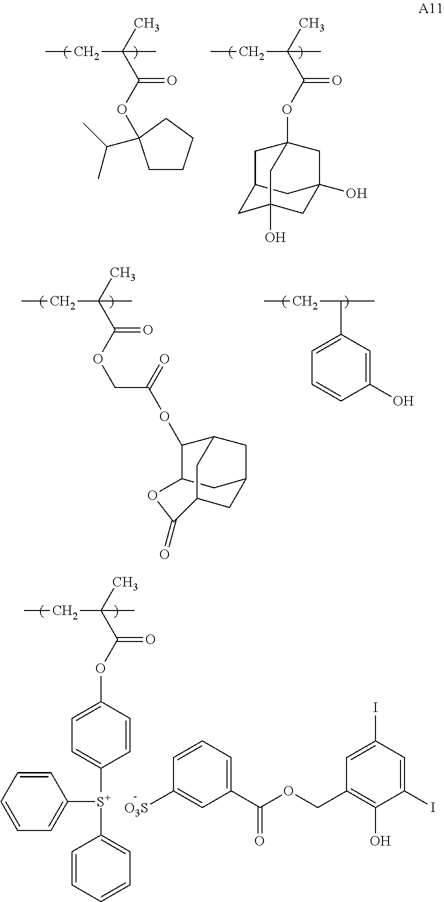

### Example 37 [Synthesis of Resin A12]

The monomer (a1-2-6), the monomer (a2-C-3), the monomer (a3-4-2), the monomer (a2-2-4a) and the monomer (I-5151) were used as monomers and mixed in a molar ratio [monomer (a1-2-6):monomer (a2-C-3):monomer (a3-4-2):monomer (a2-2-4a):monomer (I-5151)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A12 with a weight average molecular weight of about 5.2×10³ in a yield of 80%. This resin A12 has the following structural units.

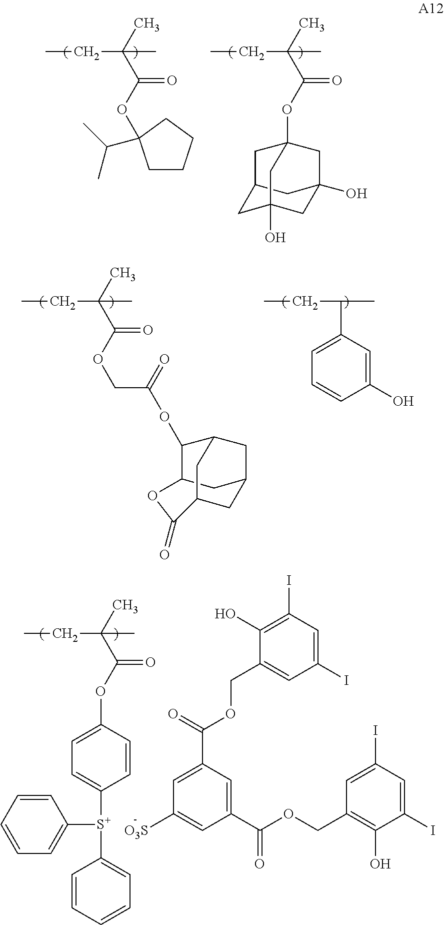

### Example 38 [Synthesis of Resin A13]

The monomer (a1-2-6), the monomer (a2-C-3), the monomer (a3-4-2), the monomer (a2-2-4a) and the monomer (I-5201) were used as monomers and mixed in a molar ratio [monomer (a1-2-6):monomer (a2-C-3):monomer (a3-4-2):monomer (a2-2-4a):monomer (I-5201)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A13 with a weight average molecular weight of about 5.2×10³ in a yield of 81%. This resin A13 has the following structural units.

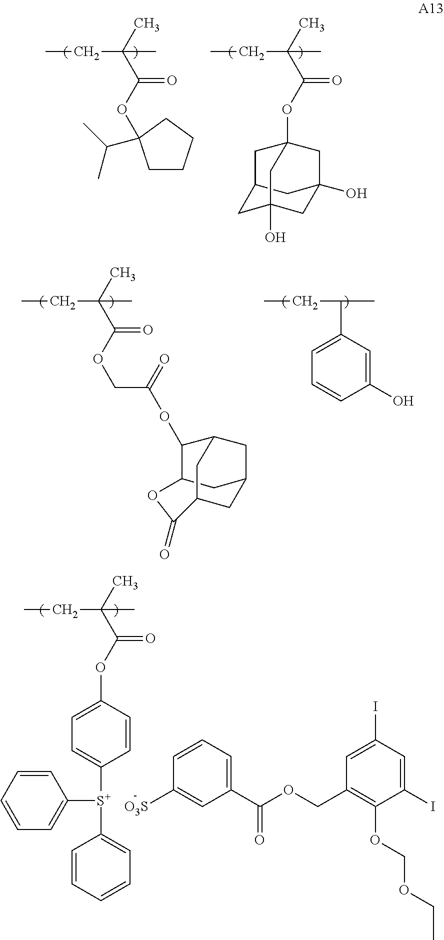

### Example 39 [Synthesis of Resin A14]

The monomer (a1-2-6), the monomer (a2-C-3), the monomer (a3-4-2), the monomer (a2-2-4a) and the monomer (I-5301) were used as monomers and mixed in a molar ratio [monomer (a1-2-6):monomer (a2-C-3):monomer (a3-4-2):monomer (a2-2-4a):monomer (I-5301)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A14 with a weight average molecular weight of about 5.1×10³ in a yield of 75%. This resin A14 has the following structural units.

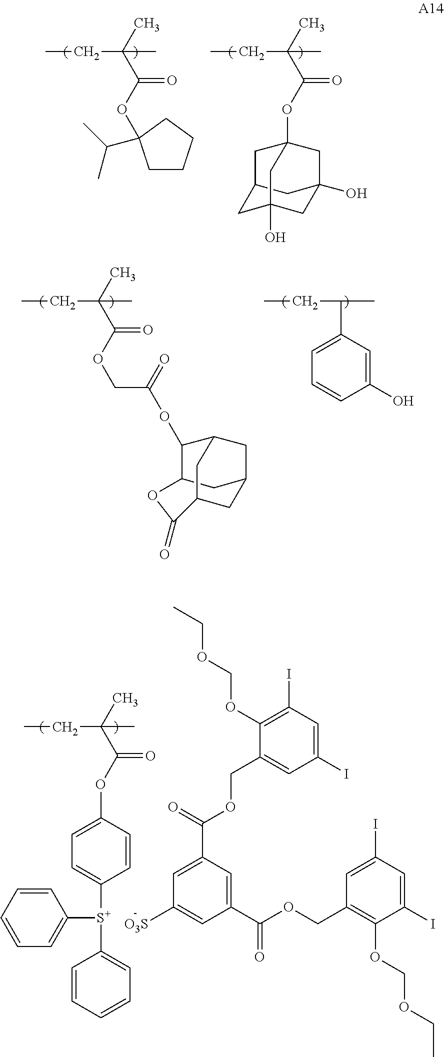

### Example 40 [Synthesis of Resin A15]

The monomer (a1-2-6), the monomer (a2-C-3), the monomer (a3-4-2), the monomer (a2-2-4a) and the monomer (I-5451) were used as monomers and mixed in a molar ratio [monomer (a1-2-6):monomer (a2-C-3):monomer (a3-4-2):monomer (a2-2-4a):monomer (I-5451)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A15 with a weight average molecular weight of about 5.4×10³ in a yield of 84%. This resin A15 has the following structural units.

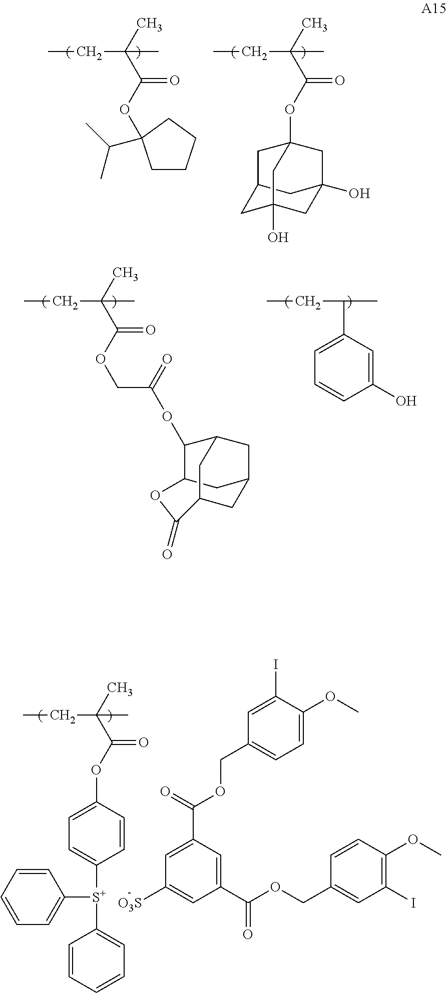

### Example 41 [Synthesis of Resin A16]

The monomer (a1-2-6), the monomer (a2-C-3), the monomer (a3-4-2), the monomer (a2-2-4a) and the monomer (I-5551) were used as monomers and mixed in a molar ratio [monomer (a1-2-6):monomer (a2-C-3):monomer (a3-4-2):monomer (a2-2-4a):monomer (I-5551)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A16 with a weight average molecular weight of about 5.5×10³ in a yield of 82%. This resin A16 has the following structural units.

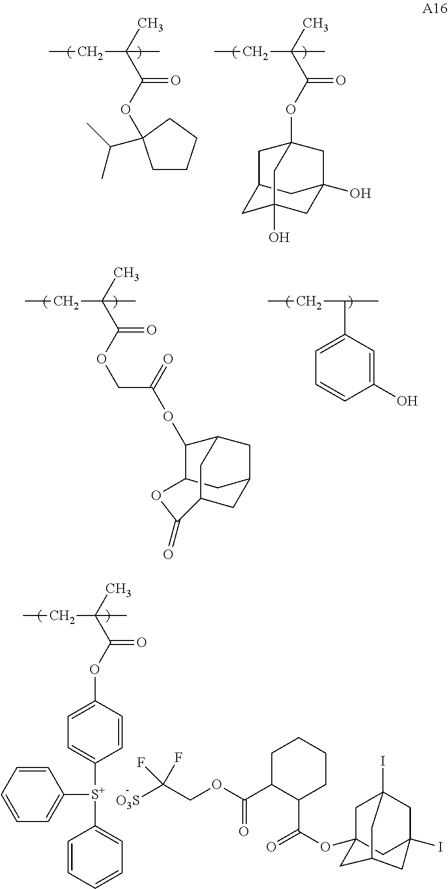

### Example 42 [Synthesis of Resin A17]

The monomer (a1-2-6), the monomer (a2-C-3), the monomer (a3-4-2), the monomer (a2-2-4a) and the monomer (I-5601) were used as monomers and mixed in a molar ratio [monomer (a1-2-6):monomer (a2-C-3):monomer (a3-4-2):monomer (a2-2-4a):monomer (I-5601)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A17 with a weight average molecular weight of about 5.6×10³ in a yield of 75%. This resin A17 has the following structural units.

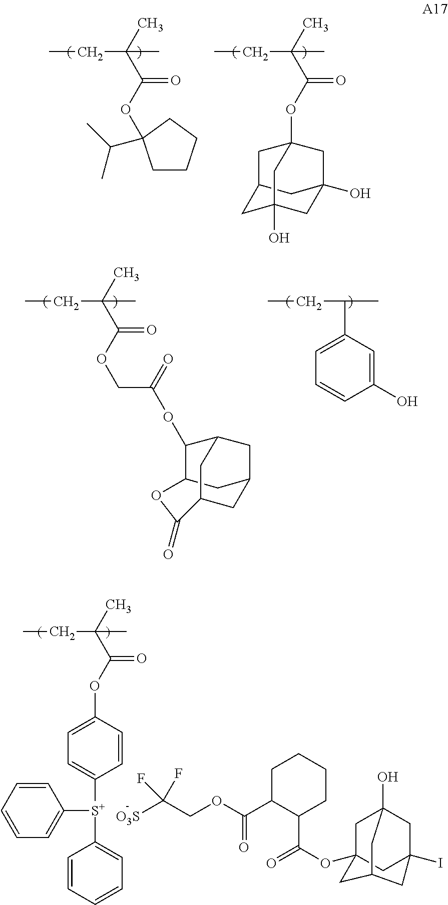

### Example 43 [Synthesis of Resin A18]

The monomer (a1-2-6), the monomer (a2-C-3), the monomer (a3-4-2), the monomer (a2-2-4a) and the monomer (I-5701) were used as monomers and mixed in a molar ratio [monomer (a1-2-6):monomer (a2-C-3):monomer (a3-4-2):monomer (a2-2-4a):monomer (I-5701)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A18 with a weight average molecular weight of about 5.4×10³ in a yield of 80%. This resin A18 has the following structural units.

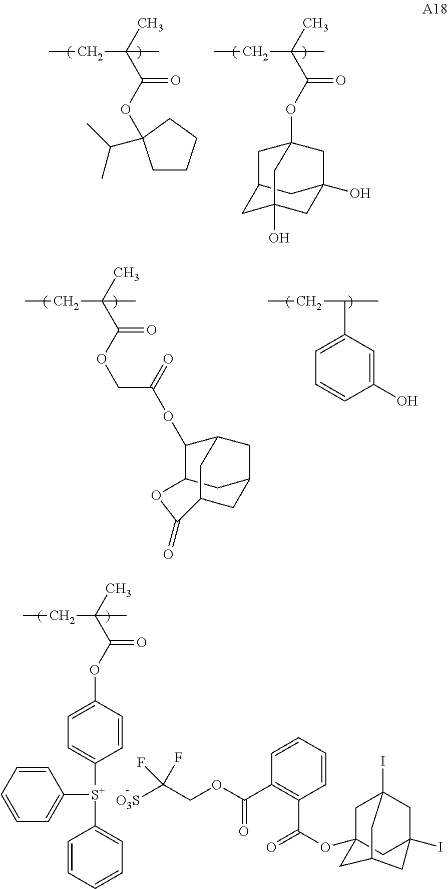

### Example 44 [Synthesis of Resin A19]

The monomer (a1-2-6), the monomer (a2-C-3), the monomer (a3-4-2), the monomer (a2-2-4a) and the monomer (I-32) were used as monomers and mixed in a molar ratio [monomer (a1-2-6):monomer (a2-C-3):monomer (a3-4-2):monomer (a2-2-4a):monomer (I-32)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A19 with a weight average molecular weight of about 5.5×10³ in a yield of 79%. This resin A19 has the following structural units.

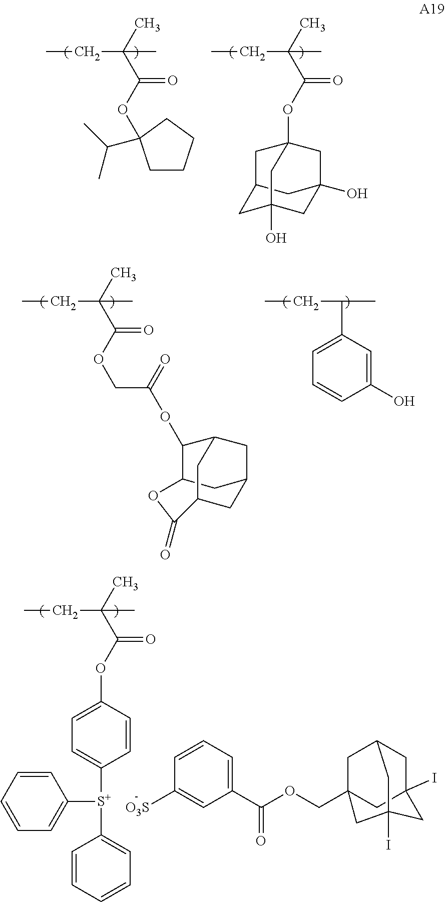

### Example 45 [Synthesis of Resin A20]

The monomer (a1-2-6), the monomer (a2-C-3), the monomer (a3-4-2), the monomer (a2-2-4a) and the monomer (I-40) were used as monomers and mixed in a molar ratio [monomer (a1-2-6):monomer (a2-C-3):monomer (a3-4-2):monomer (a2-2-4a):monomer (I-40)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A20 with a weight average molecular weight of about 5.4×10³ in a yield of 77%. This resin A20 has the following structural units.

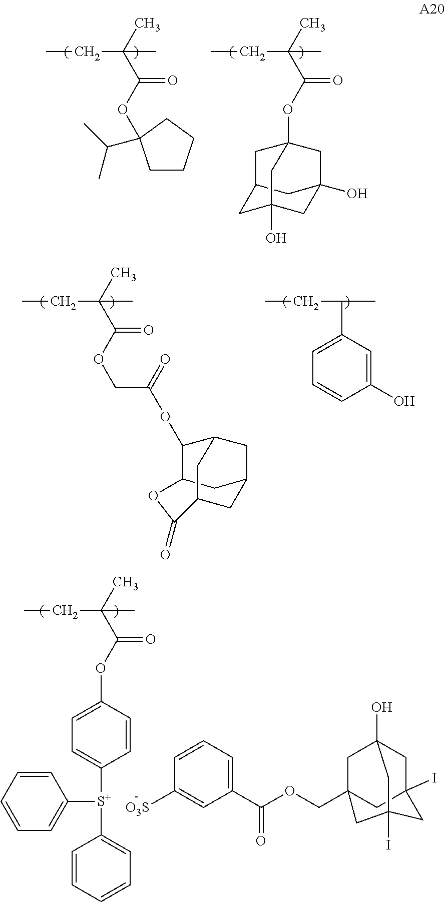

### Example 46 [Synthesis of Resin A21]

The monomer (a1-2-6), the monomer (a2-C-3), the monomer (a3-4-2), the monomer (a2-2-4a) and the monomer (I-6001) were used as monomers and mixed in a molar ratio [monomer (a1-2-6):monomer (a2-C-3):monomer (a3-4-2):monomer (a2-2-4a):monomer (I-6001)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A21 with a weight average molecular weight of about 5.2×10³ in a yield of 73%. This resin A21 has the following structural units.

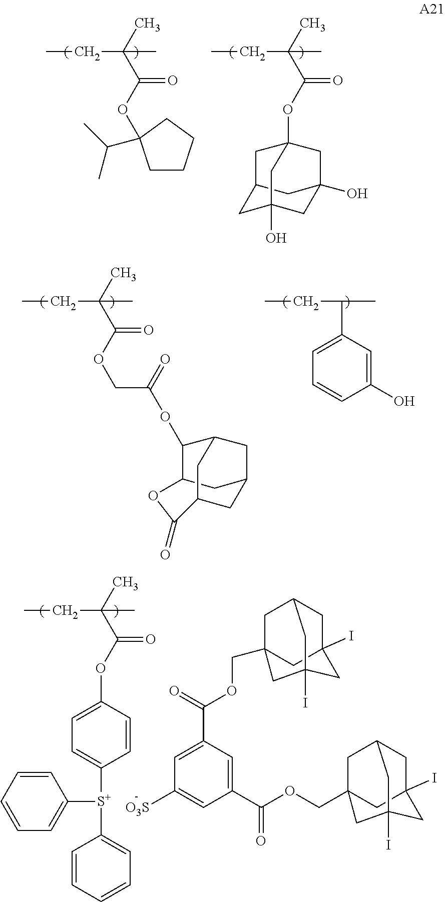

### Example 47 [Synthesis of Resin A22]

The monomer (a1-2-6), the monomer (a2-C-3), the monomer (a3-4-2), the monomer (a2-2-4a) and the monomer (I-6051) were used as monomers and mixed in a molar ratio [monomer (a1-2-6):monomer (a2-C-3):monomer (a3-4-2):monomer (a2-2-4a):monomer (I-6051)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A22 with a weight average molecular weight of about 5.3×10³ in a yield of 71%. This resin A22 has the following structural units.

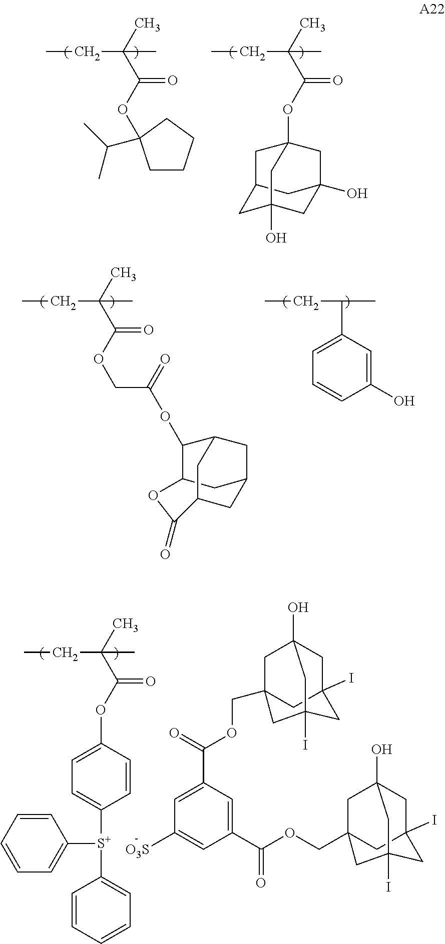

### Example 48 [Synthesis of Resin A23]

The monomer (a1-2-6), the monomer (a2-C-3), the monomer (a3-4-2), the monomer (a2-2-4a) and the monomer (I-57) were used as monomers and mixed in a molar ratio [monomer (a1-2-6):monomer (a2-C-3):monomer (a3-4-2):monomer (a2-2-4a):monomer (I-57)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A23 with a weight average molecular weight of about 5.6×10³ in a yield of 79%. This resin A23 has the following structural units.

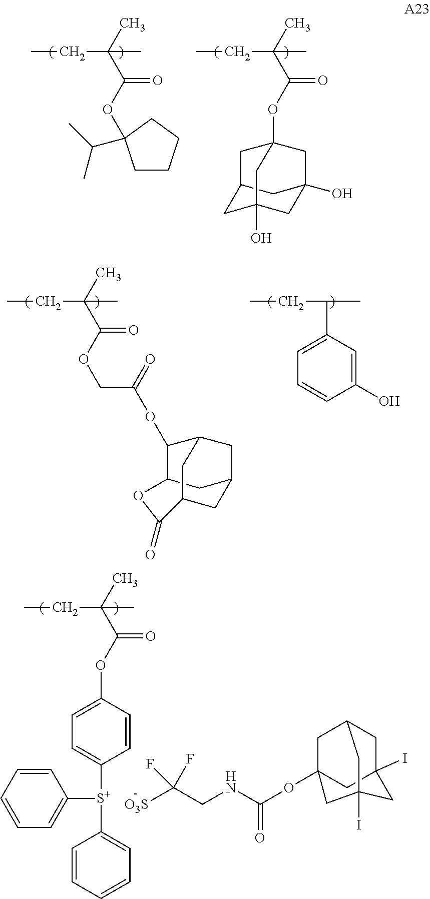

### Example 49 [Synthesis of Resin A24]

The monomer (a1-2-6), the monomer (a2-C-3), the monomer (a3-4-2), the monomer (a2-2-4a) and the monomer (I-6061) were used as monomers and mixed in a molar ratio [monomer (a1-2-6):monomer (a2-C-3):monomer (a3-4-2):monomer (a2-2-4a):monomer (I-6061)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A24 with a weight average molecular weight of about 5.4×10³ in a yield of 75%. This resin A24 has the following structural units.

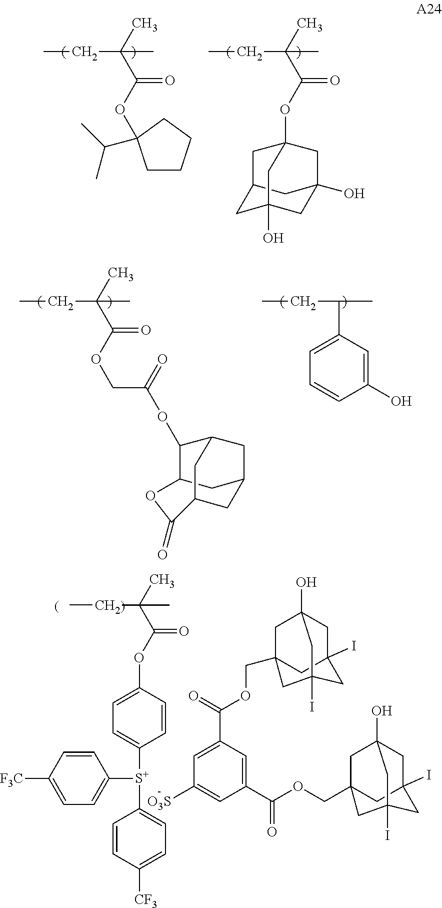

### Example 50 [Synthesis of Resin A25]

The monomer (a1-2-6), the monomer (a2-C-3), the monomer (a3-4-2), the monomer (a2-2-4a) and the monomer (I-6099) were used as monomers and mixed in a molar ratio [monomer (a1-2-6):monomer (a2-C-3):monomer (a3-4-2):monomer (a2-2-4a):monomer (I-6099)] of 50:3:10:27:10. Furthermore, propylene glycol monomethyl ether was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin A25 with a weight average molecular weight of about 5.3×10³ in a yield of 72%. This resin A25 has the following structural units.

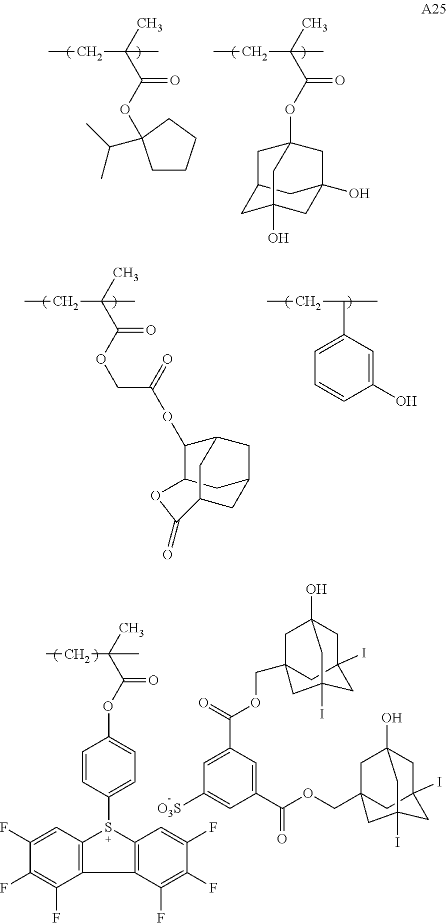

### Synthesis Example 1 [Synthesis of Resin AX1]

The monomer (a1-2-6), the monomer (a2-1-3), the monomer (a3-4-2), the monomer (a2-2-4a) and the monomer (IX-1) were used as monomers and mixed in a molar ratio [monomer (a1-2-6):monomer (a2-1-3):monomer (a3-4-2):monomer (a2-2-4a):monomer (IX-1)] of 50:3:10:27:10. Furthermore, methyl isobutyl ketone was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin AX1 with a weight average molecular weight of about 5.4×10³ in a yield of 88%. This resin AX1 has the following structural units.

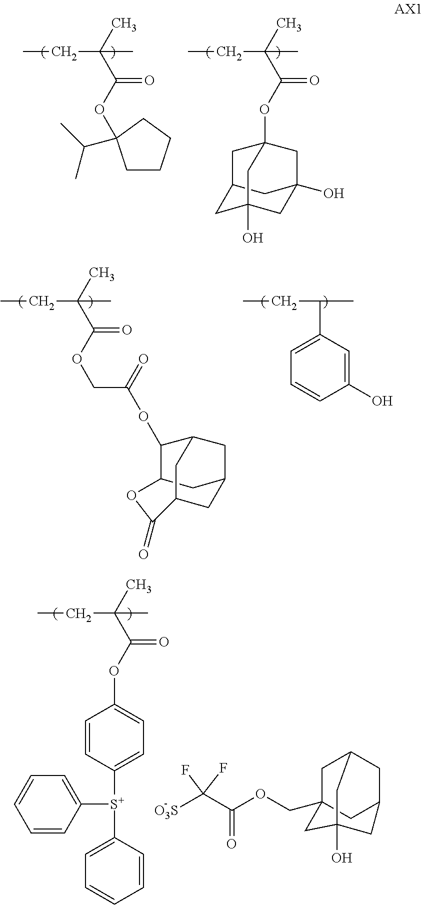

### Synthesis Example 2 [Synthesis of Resin AX2]

The monomer (a1-2-6), the monomer (a2-1-3), the monomer (a3-4-2), the monomer (a2-2-4a) and the monomer (IX-2) were used as monomers and mixed in a molar ratio [monomer (a1-2-6):monomer (a2-1-3):monomer (a3-4-2):monomer (a2-2-4a):monomer (IX-2)] of 50:3:10:27:10. Furthermore, methyl isobutyl ketone was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin AX2 with a weight average molecular weight of about 5.3×10³ in a yield of 90%. This resin AX2 has the following structural units.

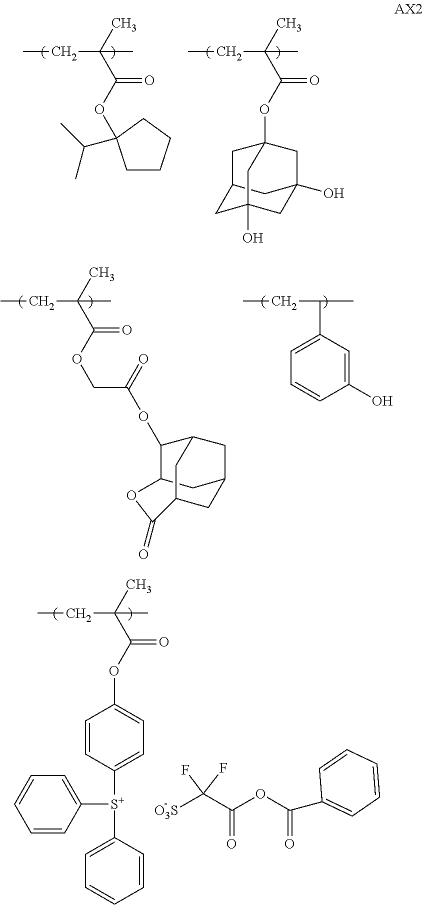

### Synthesis Example 3 [Synthesis of Resin AA1]

The monomer (a1-2-6), the monomer (a2-1-3), the monomer (a3-4-2) and the monomer (a2-2-4a) were used as monomers and mixed in a molar ratio [monomer (a1-2-6):monomer (a2-1-3):monomer (a3-4-2):monomer (a2-2-4a)] of 55:3:15:27. Furthermore, methyl isobutyl ketone was mixed into this monomer mixture in an amount of 1.5 times the total mass of all monomers. To the resulting mixture, azobisisobutyronitrile and azobis(2,4-dimethylvaleronitrile) were added as initiators in amounts of 1.2 mol % and 3.6 mol %, respectively, with respect to the total monomer amount, and the mixture was heated at 73° C. for about 5 hours. Then, the polymerization reaction liquid was poured into a large amount of n-heptane to precipitate the resin, which was then filtered and collected to provide a resin AA1 with a weight average molecular weight of about 5.6×10³ in a yield of 88%. This resin AA1 has the following structural units.

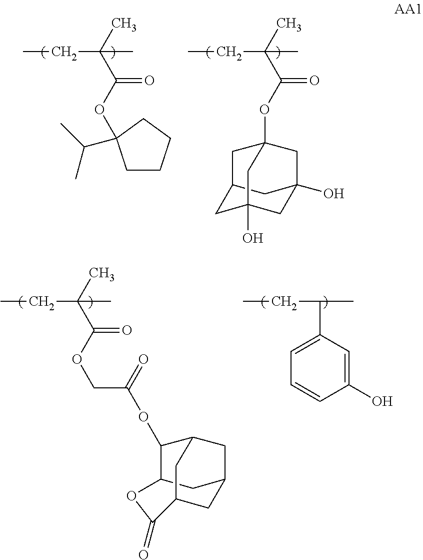

### <Preparation of Resist Composition>

As shown in Table 2, the following components were mixed and the resulting mixture was filtered through a fluororesin filter with a pore size of 0.2 μm to prepare a resist composition.

TABLE 2

| Resist | | Acid generator | | | |
|---|---|---|---|---|---|
| composition | Resin | (B) | Salt (I) | Quencher (C) | PB/PEB |
| Composition 1 | AA1 = 10 | — | I-1 = 1.5 parts | C1 = 0.35 parts | 100° C./130° C. |
| | parts | | | | |
| Composition 2 | AA1 = 10 | — | I-43 = 1.5 parts | C1 = 0.35 parts | 100° C./130° C. |
| | parts | | | | |
| Composition 3 | A1 = 10 parts | B1-25 = 0.5 parts | — | C1 = 0.35 parts | 100° C./130° C. |
| Composition 4 | A1 = 10 parts | — | I-1 = 0.5 parts | C1 = 0.35 parts | 100° C./130° C. |
| Composition 5 | A1 = 10 parts | — | — | C1 = 0.35 parts | 100° C./130° C. |
| Composition 6 | A2 = 10 parts | B1-25 = 0.5 parts | — | C1 = 0.35 parts | 100° C./130° C. |
| Composition 7 | A2 = 10 parts | — | I-3 = 0.5 parts | C1 = 0.35 parts | 100° C./130° C. |
| Composition 8 | A2 = 10 parts | — | — | C1 = 0.35 parts | 100° C./130° C. |
| Composition 9 | A3 = 10 parts | B1-25 = 0.5 parts | — | C1 = 0.35 parts | 100° C./130° C. |
| Composition 10 | A3 = 10 parts | — | I-23 = 0.5 parts | C1 = 0.35 parts | 100° C./130° C. |
| Composition 11 | A3 = 10 parts | — | — | C1 = 0.35 parts | 100° C./130° C. |
| Composition 12 | A4 = 10 parts | B1-25 = 0.5 parts | — | C1 = 0.35 parts | 100° C./130° C. |
| Composition 13 | A4 = 10 parts | — | I-28 = 0.5 parts | C1 = 0.35 parts | 100° C./130° C. |
| Composition 14 | A4 = 10 parts | — | — | C1 = 0.35 parts | 100° C./130° C. |
| Composition 15 | A5 = 10 parts | B1-25 = 0.5 parts | — | C1 = 0.35 parts | 100° C./130° C. |
| Composition 16 | A5 = 10 parts | — | I-43 = 0.5 parts | C1 = 0.35 parts | 100° C./130° C. |
| Composition 17 | A5 = 10 parts | — | — | C1 = 0.35 parts | 100° C./130° C. |
| Composition 18 | A6 = 10 parts | B1-25 = 0.5 parts | — | C1 = 0.35 parts | 100° C./130° C. |
| Composition 19 | A6 = 10 parts | — | I-603 = 0.5 parts | C1 = 0.35 parts | 100° C./130° C. |
| Composition 20 | A6 = 10 parts | — | — | C1 = 0.35 parts | 100° C./130° C. |
| Composition 21 | A7 = 10 parts | B1-25 = 0.5 parts | — | C1 = 0.35 parts | 100° C./130° C. |
| Composition 22 | A7 = 10 parts | — | I-7 = 0.5 parts | C1 = 0.35 parts | 100° C./130° C. |
| Composition 23 | A7 = 10 parts | — | — | C1 = 0.35 parts | 100° C./130° C. |
| Composition 24 | A8 = 10 parts | B1-25 = 0.5 parts | — | C1 = 0.35 parts | 100° C./130° C. |
| Composition 25 | A8 = 10 parts | — | I-1107 = 0.5 parts | C1 = 0.35 parts | 100° C./130° C. |
| Composition 26 | A8 = 10 parts | — | — | C1 = 0.35 parts | 100° C./130° C. |
| Composition 27 | A9 = 10 parts | B1-25 = 0.5 parts | — | C1 = 0.35 parts | 100° C./130° C. |
| Composition 28 | A9 = 10 parts | — | I-4507 = 0.5 parts | C1 = 0.35 parts | 100° C./130° C. |
| Composition 29 | A9 = 10 parts | — | — | C1 = 0.35 parts | 100° C./130° C. |
| Composition 30 | A10 = 10 | B1-25 = 0.5 parts | — | C1 = 0.35 parts | 100° C./130° C. |
| | parts | | | | |
| Composition 31 | A10 = 10 | — | I-10 = 0.5 parts | C1 = 0.35 parts | 100° C./130° C. |
| | parts | | | | |
| Composition 32 | A10 = 10 | — | — | C1 = 0.35 parts | 100° C./130° C. |
| | parts | | | | |
| Composition 33 | A11 = 10 | B1-25 = 0.5 parts | — | C1 = 0.35 parts | 100° C./130° C. |
| | parts | | | | |
| Composition 34 | A11 = 10 | — | I-5001 = 0.5 parts | C1 = 0.35 parts | 100° C./130° C. |
| | parts | | | | |
| Composition 35 | A11 = 10 | — | — | C1 = 0.35 parts | 100° C./130° C. |
| | parts | | | | |
| Composition 36 | A12 = 10 | B1-25 = 0.5 parts | — | C1 = 0.35 parts | 100° C./130° C. |
| | parts | | | | |
| Composition 37 | A12 = 10 | — | I-5151 = 0.5 parts | C1 = 0.35 parts | 100° C./130° C. |
| | parts | | | | |
| Composition 38 | A12 = 10 | — | — | C1 = 0.35 parts | 100° C./130° C. |
| | parts | | | | |
| Composition 39 | A13 = 10 | B1-25 = 0.5 parts | — | C1 = 0.35 parts | 100° C./130° C. |
| | parts | | | | |
| Composition 40 | A13 = 10 | — | I-5201 = 0.5 parts | C1 = 0.35 parts | 100° C./130° C. |
| | parts | | | | |
| Composition 41 | A13 = 10 | — | — | C1 = 0.35 parts | 100° C./130° C. |
| | parts | | | | |
| Composition 42 | A14 = 10 | B1-25 = 0.5 parts | — | C1 = 0.35 parts | 100° C./130° C. |
| | parts | | | | |
| Composition 43 | A14 = 10 | — | I-5301 = 0.5 parts | C1 = 0.35 parts | 100° C./130° C. |
| | parts | | | | |
| Composition 44 | A14 = 10 | — | — | C1 = 0.35 parts | 100° C./130° C. |
| | parts | | | | |
| Composition 45 | A15 = 10 | B1-25 = 0.5 parts | — | C1 = 0.35 parts | 100° C./130° C. |
| | parts | | | | |
| Composition 46 | A15 = 10 | — | I-5451 = 0.5 parts | C1 = 0.35 parts | 100° C./130° C. |
| | parts | | | | |
| Composition 47 | A15 = 10 | — | — | C1 = 0.35 parts | 100° C./130° C. |
| | parts | | | | |
| Composition 48 | A16 = 10 | B1-25 = 0.5 parts | — | C1 = 0.35 parts | 100° C./130° C. |
| | parts | | | | |
| Composition 49 | A16 = 10 | — | I-5551 = 0.5 parts | C1 = 0.35 parts | 100° C./130° C. |
| | parts | | | | |
| Composition 50 | A16 = 10 | — | — | C1 = 0.35 parts | 100° C./130° C. |
| | parts | | | | |
| Composition 51 | A17 = 10 | B1-25 = 0.5 parts | — | C1 = 0.35 parts | 100° C./130° C. |
| | parts | | | | |
| Composition 52 | A17 = 10 | — | I-5601 = 0.5 parts | C1 = 0.35 parts | 100° C./130° C. |
| | parts | | | | |
| Composition 53 | A17 = 10 | — | — | C1 = 0.35 parts | 100° C./130° C. |
| | parts | | | | |
| Composition 54 | A18 = 10 | B1-25 = 0.5 parts | — | C1 = 0.35 parts | 100° C./130° C. |
| | parts | | | | |
| Composition 55 | A18 = 10 | — | I-5701 = 0.5 parts | C1 = 0.35 parts | 100° C./130° C. |
| | parts | | | | |
| Composition 56 | A18 = 10 | — | — | C1 = 0.35 parts | 100° C./130° C. |
| | parts | | | | |
| Composition 57 | A19 = 10 | — | I-32 = 0.5 parts | C1 = 0.35 parts | 100° C./130° C. |
| | parts | | | | |
| Composition 58 | A19 = 10 | — | — | C1 = 0.35 parts | 100° C./130° C. |
| | parts | | | | |
| Composition 59 | A20 = 10 | — | I-40 = 0.5 parts | C1 = 0.35 parts | 100° C./130° C. |
| | parts | | | | |
| Composition 60 | A20 = 10 | — | — | C1 = 0.35 parts | 100° C./130° C. |
| | parts | | | | |
| Composition 61 | A21 = 10 | — | I-6001 = 0.5 parts | C1 = 0.35 parts | 100° C./130° C. |
| | parts | | | | |
| Composition 62 | A21 = 10 | — | — | C1 = 0.35 parts | 100° C./130° C. |
| | parts | | | | |
| Composition 63 | A22 = 10 | — | I-6051 = 0.5 parts | C1 = 0.35 parts | 100° C./130° C. |
| | parts | | | | |
| Composition 64 | A22 = 10 | — | — | C1 = 0.35 parts | 100° C./130° C. |
| | parts | | | | |
| Composition 65 | A23 = 10 | — | I-57 = 0.5 parts | C1 = 0.35 parts | 100° C./130° C. |
| | parts | | | | |
| Composition 66 | A23 = 10 | — | — | C1 = 0.35 parts | 100° C./130° C. |
| | parts | | | | |
| Composition 67 | A24 = 10 | — | I-6061 = 0.5 parts | C1 = 0.35 parts | 100° C./130° C. |
| | parts | | | | |
| Composition 68 | A24 = 10 | — | — | C1 = 0.35 parts | 100° C./130° C. |
| | parts | | | | |
| Composition 69 | A25 = 10 | — | I-6099 = 0.5 parts | C1 = 0.35 parts | 100° C./130° C. |
| | parts | | | | |
| Composition 70 | A25 = 10 | — | — | C1 = 0.35 parts | 100° C./130° C. |
| | parts | | | | |
| Comparative | AA1 = 10 | IX-1 = 1.5 parts | — | C1 = 0.35 parts | 100° C./130° C. |
| Composition 1 | parts | | | | |
| Comparative | AX1 = 10 | B1-25 = 0.5 parts | — | C1 = 0.35 parts | 100° C./130° C. |
| Composition 2 | parts | | | | |
| Comparative | AX1 = 10 | IX-1 = 0.5 parts | — | C1 = 0.35 parts | 100° C./130° C. |
| Composition 3 | parts | | | | |
| Comparative | AX1 = 10 | — | — | C1 = 0.35 parts | 100° C./130° C. |
| Composition 4 | parts | | | | |
| Comparative | AA1 = 10 | IX-2 = 1.5 parts | — | C1 = 0.35 parts | 100° C./130° C. |
| Composition 5 | parts | | | | |
| Comparative | AX2 = 10 | B1-25 = 0.5 parts | — | C1 = 0.35 parts | 100° C./130° C. |
| Composition 6 | parts | | | | |
| Comparative | AX2 = 10 | IX-2 = 0.5 parts | — | C1 = 0.35 parts | 100° C./130° C. |
| Composition 7 | parts | | | | |
| Comparative | AX2 = 10 | — | — | C1 = 0.35 parts | 100° C./130° C. |
| Composition 8 | parts | | | | |

### <Resin>

A1 to A25, AA1, AX1 and AX2: Resin A1 to Resin A25, Resin AA1, Resin AX1 and Resin AX2

### <Salt (I)>

I-1: salt represented by formula (I-1)
I-3: salt represented by formula (I-3)
I-7: salt represented by formula (I-7)
I-10: salt represented by formula (I-10)
I-23: salt represented by formula (I-23)
I-28: salt represented by formula (I-28)
I-32: salt represented by formula (I-32)
I-40: salt represented by formula (I-40)
I-43: salt represented by formula (I-43)
I-57: salt represented by formula (I-57)
I-603: salt represented by formula (I-603)
I-1107: salt represented by formula (I-1107)
I-4507: salt represented by formula (I-4507)
I-5001: salt represented by formula (I-5001)
I-5151: salt represented by formula (I-5151)
I-5201: salt represented by formula (I-5201)
I-5301: salt represented by formula (I-5301)
I-5451: salt represented by formula (I-5451)
I-5551: salt represented by formula (I-5551)
I-5601: salt represented by formula (I-5601)
I-5701: salt represented by formula (I-5701)
I-6001: salt represented by formula (I-6001)
I-6051: salt represented by formula (I-6051)
I-6061: salt represented by formula (I-6061)
I-6099: salt represented by formula (I-6099)

### <Acid Generating Agent (B)>

B1-25: salt represented by formula (B1-25); synthesized by the method disclosed in Japanese Patent Laid-Open No. 2011-126869

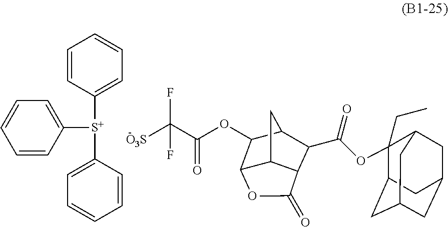

IX-1: synthesized by the method disclosed in Japanese Patent Laid-Open No. 2011-037836
IX-2: synthesized by the method disclosed in Japanese Patent Laid-Open No. 2010-132852

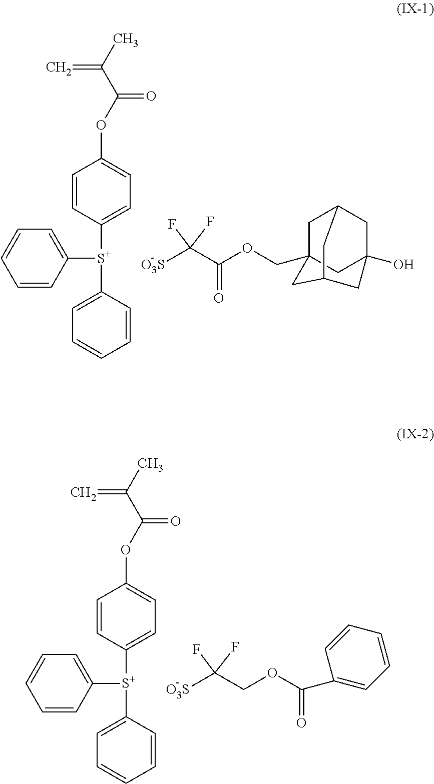

### <Quencher (C)>

C1: synthesized by the method disclosed in Japanese Patent Laid-Open No. 2011-39502

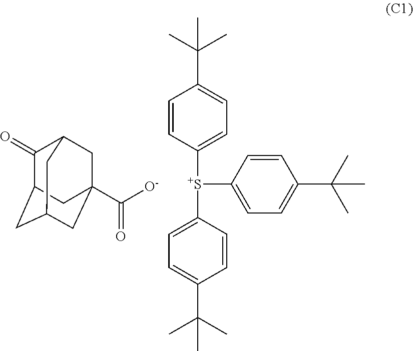 [SMILES: O=C1C2CC3CC1CC(C(=O)[O-])(C3)C2 ; CC(C)(C)c1ccc([S+](c2ccc(C(C)(C)C)cc2)c2ccc(C(C)(C)C)cc2)cc1]

### <Solvent>

Propylene glycol monomethyl ether acetate 400 parts
Propylene glycol monomethyl ether 100 parts
γ-butyrolactone 5 parts

### (Electron Beam Exposure Evaluation of Resist Composition: Organic Solvent Development)

A 6-inch silicon wafer was treated with hexamethyldisilazane on a direct hot plate at 90° C. for 60 seconds. The resist composition was spin-coated onto this silicon wafer such that the thickness of the composition layer was 0.04 μm. Then, the wafer was pre-baked on the direct hot plate for 60 seconds at the temperature shown in the “PB” column of Table 2 to form a composition layer. An electron beam drawing machine (ELS-F125 125 keV, manufactured by ELIONIX INC.) was used to perform direct drawing of a contact hole pattern (hole pitch 40 nm/hole diameter 17 nm) for the composition layer formed on the wafer by gradually changing the exposure dose.

After exposure, post-exposure baking was performed on a hot plate for 60 seconds at the temperature shown in the “PEB” column of Table 2. The composition layer on the silicon wafer was then developed by the dynamic dispense method for 20 seconds at 23° C. using butyl acetate (manufactured by Tokyo Chemical Industry Co., Ltd.) as the developer, resulting in a resist pattern.

In the resist pattern obtained after development, the exposure dose that resulted in a hole diameter of 17 nm was defined as the effective sensitivity.

### <CD Uniformity (CDU) Evaluation>

At the effective sensitivity, the hole diameter of the pattern formed with a hole diameter of 17 nm was measured 24 times per hole, and the average value thereof was taken as the average hole diameter of one hole. The average hole diameter of the pattern formed with a hole diameter of 17 nm was measured at 400 locations within the same wafer, and the standard deviation was determined from this population.

The results are shown in Table 3. The values in the table indicate the standard deviation (nm).

TABLE 3

| | Resist composition | CDU |
|---|---|---|
| Example 51 | Composition 1 | 2.44 |
| Example 52 | Composition 2 | 2.41 |
| Example 53 | Composition 3 | 2.59 |
| Example 54 | Composition 4 | 2.58 |
| Example 55 | Composition 5 | 2.61 |
| Example 56 | Composition 6 | 2.55 |
| Example 57 | Composition 7 | 2.53 |
| Example 58 | Composition 8 | 2.58 |
| Example 59 | Composition 9 | 2.64 |
| Example 60 | Composition 10 | 2.62 |
| Example 61 | Composition 11 | 2.66 |
| Example 62 | Composition 12 | 2.62 |
| Example 63 | Composition 13 | 2.61 |
| Example 64 | Composition 14 | 2.65 |
| Example 65 | Composition 15 | 2.52 |
| Example 66 | Composition 16 | 2.49 |
| Example 67 | Composition 17 | 2.54 |
| Example 68 | Composition 18 | 2.54 |
| Example 69 | Composition 19 | 2.52 |
| Example 70 | Composition 20 | 2.57 |
| Example 71 | Composition 21 | 2.54 |
| Example 72 | Composition 22 | 2.51 |
| Example 73 | Composition 23 | 2.55 |
| Example 74 | Composition 24 | 2.42 |
| Example 75 | Composition 25 | 2.38 |
| Example 76 | Composition 26 | 2.45 |
| Example 77 | Composition 27 | 2.33 |
| Example 78 | Composition 28 | 2.30 |
| Example 79 | Composition 29 | 2.35 |
| Example 80 | Composition 30 | 2.48 |
| Example 81 | Composition 31 | 2.45 |
| Example 82 | Composition 32 | 2.50 |
| Example 83 | Composition 33 | 2.58 |
| Example 84 | Composition 34 | 2.56 |
| Example 85 | Composition 35 | 2.60 |
| Example 86 | Composition 36 | 2.46 |
| Example 87 | Composition 37 | 2.40 |
| Example 88 | Composition 38 | 2.47 |
| Example 89 | Composition 39 | 2.56 |
| Example 90 | Composition 40 | 2.54 |
| Example 91 | Composition 41 | 2.59 |
| Example 92 | Composition 42 | 2.64 |
| Example 93 | Composition 43 | 2.39 |
| Example 94 | Composition 44 | 2.45 |
| Example 95 | Composition 45 | 2.59 |
| Example 96 | Composition 46 | 2.56 |
| Example 97 | Composition 47 | 2.65 |
| Example 98 | Composition 48 | 2.44 |
| Example 99 | Composition 49 | 2.41 |
| Example 100 | Composition 50 | 2.45 |
| Example 101 | Composition 51 | 2.40 |
| Example 102 | Composition 52 | 2.38 |
| Example 103 | Composition 53 | 2.42 |
| Example 104 | Composition 54 | 2.45 |
| Example 105 | Composition 55 | 2.42 |
| Example 106 | Composition 56 | 2.46 |
| Example 107 | Composition 57 | 2.45 |
| Example 108 | Composition 58 | 2.49 |
| Example 109 | Composition 59 | 2.39 |
| Example 110 | Composition 60 | 2.42 |
| Example 111 | Composition 61 | 2.40 |
| Example 112 | Composition 62 | 2.46 |
| Example 113 | Composition 63 | 2.32 |
| Example 114 | Composition 64 | 2.34 |
| Example 115 | Composition 65 | 2.53 |
| Example 116 | Composition 66 | 2.56 |
| Example 117 | Composition 67 | 2.23 |
| Example 118 | Composition 68 | 2.26 |
| Example 119 | Composition 69 | 2.19 |
| Example 120 | Composition 70 | 2.22 |
| Comparative Example 1 | Comparative Composition 1 | 2.80 |
| Comparative Example 2 | Comparative Composition 2 | 2.79 |
| Comparative Example 3 | Comparative Composition 3 | 2.78 |
| Comparative Example 4 | Comparative Composition 4 | 2.85 |
| Comparative Example 5 | Comparative Composition 5 | 2.93 |
| Comparative Example 6 | Comparative Composition 6 | 2.95 |
| Comparative Example 7 | Comparative Composition 7 | 2.98 |
| Comparative Example 8 | Comparative Composition 8 | 3.03 |

Compared with the comparative compositions 1 to 8, the standard deviation in the compositions 1 to 70 was smaller, and the CD uniformity (CDU) evaluation was better.

### INDUSTRIAL APPLICABILITY

The salt of the present invention and the resist composition containing the salt have good CD uniformity (CDU) and are useful for semiconductor microfabrication.

1. A resist composition comprising a salt represented by formula (I) or a resin that includes a structural unit represented by formula (IP): 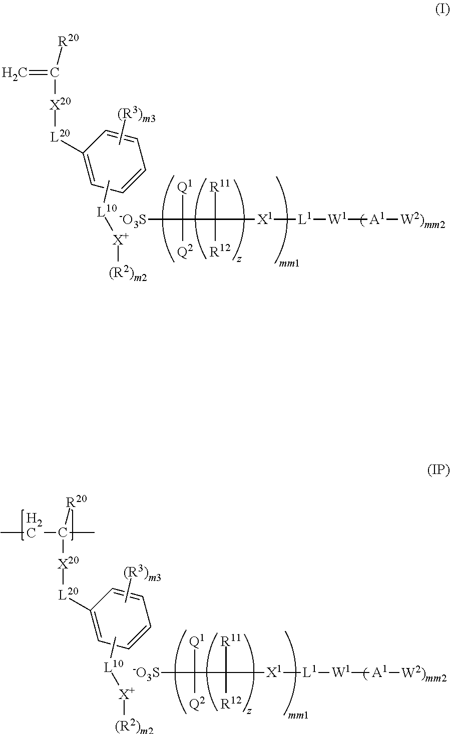
wherein
Q¹, Q², R¹¹, and R¹² each independently represent a hydrogen atom, a fluorine atom, an alkyl group having 1 to 6 carbon atoms, or a perfluoroalkyl group having 1 to 6 carbon atoms;
z represents an integer of 0 to 6, and when z is 2 or larger, a plurality of R¹¹ and R¹² may be the same or different from each other;
X¹ represents a single bond, —O—, —CO—, —S—, —SO—, —SO₂—, —NR⁷—, or a combined group of these groups;
mm1 represents 0 or 1;
L¹ represents a single bond, or a hydrocarbon group having 1 to 40 carbon atoms and optionally having a substituent, and —CH₂— contained in the hydrocarbon group is optionally replaced with —O—, —S—, —SO₂—, —SO—, —NR⁷— or —CO—;
R⁷ represents a hydrogen atom or an alkyl group having 1 to 6 carbon atoms;
W¹ represents a single bond, or a cyclic hydrocarbon group having 3 to 36 carbon atoms and optionally having a substituent, and —CH₂— contained in the cyclic hydrocarbon group is optionally replaced with —O—, —S—, —SO₂—, —SO—, —NR⁷— or —CO—;
A¹ represents a single bond, or a hydrocarbon group having 1 to 20 carbon atoms, the hydrocarbon group optionally has a substituent, and —CH₂— contained in the hydrocarbon group is optionally replaced with —O—, —S—, —SO₂—, —SO—, —NR⁷— or —CO—;
W² represents a cyclic hydrocarbon group having 3 to 36 carbon atoms and an iodine atom, —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom;
mm2 represents an integer of 1 to 5, and when mm2 is 2 or larger, a plurality of groups in parentheses may be the same or different from each other;
R² represents a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
m2 represents 1 or 2, and when m2 is 1, X is an iodine atom, and when m2 is 2, X is a sulfur atom and two R² optionally together form a ring containing a sulfur atom, where —CH₂— contained in the ring is optionally replaced with —O—, —S—, —SO₂—, —SO—, —NR⁷— or —CO—;
R³ represents a halogen atom, or a hydrocarbon group having 1 to 18 carbon atoms, the hydrocarbon group optionally has a substituent, and —CH₂— contained in the hydrocarbon group is optionally replaced with —O—, —S—, —SO₂—, —SO—, —NR⁷— or —CO—;
m3 represents an integer of 0 to 4, and when m3 is 2 or larger, a plurality of R³ may be the same or different from each other;
L¹⁰ and L²⁰ each independently represent a single bond, or a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
R²⁰ represents a hydrogen atom, a halogen atom, or an alkyl group having 1 to 6 carbon atoms and optionally having a halogen atom; and
X²⁰ represents a single bond, —O—, —CO—, —NR⁷—, —S—, —SO—, —SO₂—, a phenylene group optionally having a substituent, or a combined group of these groups.

2. The resist composition according to claim 1, wherein X²⁰ is a single bond or a group represented by any one of formulas (X²⁰-1) to (X²⁰-10): 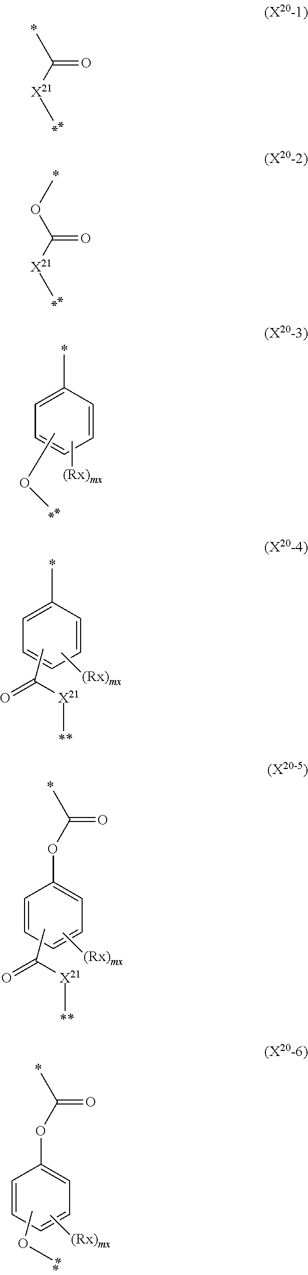 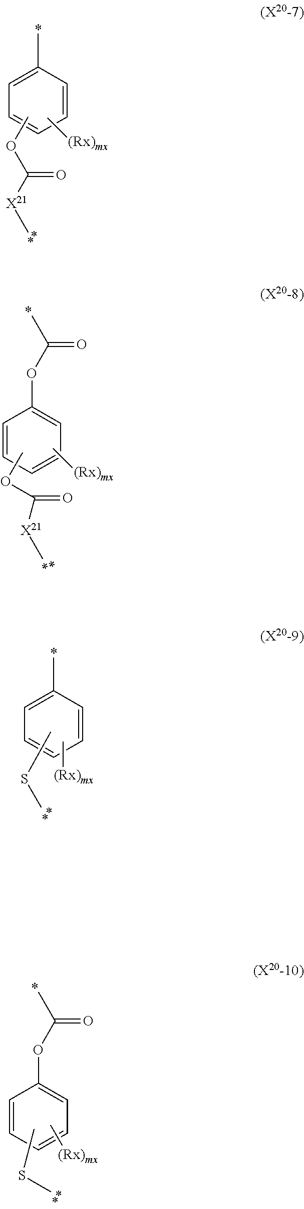
wherein
* and ** are bonding sites, and * represents a bonding site to a carbon atom to which R²⁰ is bonded;
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms;
mx represents an integer of 0 to 4; when mx is 2 or larger, a plurality of Rx may be the same or different from each other;
X²¹ represents —O— or —NR⁷—; and
R⁷ represents a hydrogen atom or an alkyl group having 1 to 6 carbon atoms.

3. The resist composition according to claim 1, wherein L¹ is a single bond, or a chain hydrocarbon group having 1 to 10 carbon atoms and optionally having a substituent, where —CH₂— contained in the chain hydrocarbon group is optionally replaced with —O—, —S—, —SO₂—, —SO—, —NR⁷— or —CO—; and
W¹ is a single bond, or a cyclic hydrocarbon group having 3 to 18 carbon atoms and optionally having a substituent, where —CH₂— contained in the cyclic hydrocarbon group is optionally replaced with —O—, —S—, —SO₂—, —SO—, —NR⁷— or —CO—.

4. The resist composition according to claim 1, wherein W² is a cyclic hydrocarbon group having 5 to 18 carbon atoms and an iodine atom, where the cyclic hydrocarbon group optionally has a substituent other than the iodine atom.

5. The resist composition according to claim 1, wherein the resist composition further comprises a resin that includes a structural unit (a1) having an acid labile group, or
the resin that includes a structural unit represented by formula (IP) further includes a structural unit (a1) having an acid labile group.

6. The resist composition according to claim 5, wherein the structural unit (a1) having an acid labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5), and a structural unit represented by formula (a1-6): 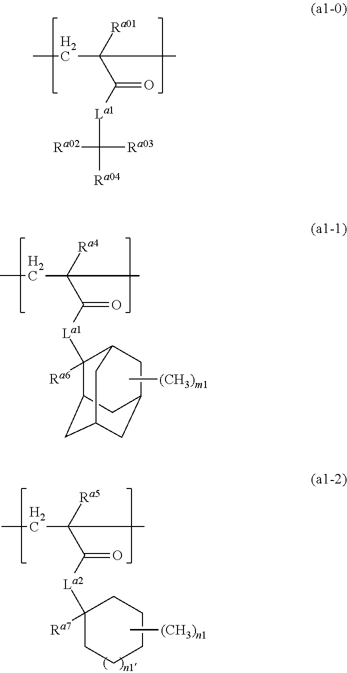
wherein
L^{a01}, L^{a1} and L^{a2} each independently represent —O— or *—O—(CH₂)ₖ₁—CO—O—, k1 represents an integer of 1 to 7, and * represents a bonding site to —CO—;
R^{a01}, R^{a4} and R^{a5} each independently represent a hydrogen atom, a halogen atom, or an alkyl group having 1 to 6 carbon atoms and optionally having a halogen atom;
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or a combined group of these groups, and the alkyl group, the alicyclic hydrocarbon group and the aromatic hydrocarbon group optionally have a halogen atom;
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or a group formed by combining these groups, and the alkyl group, the alkenyl group, the alicyclic hydrocarbon group, and the aromatic hydrocarbon group optionally have a halogen atom;
m1 represents an integer of 0 to 14;
n1 represents an integer of 0 to 14; and
n1′ represents an integer of 0 to 3, 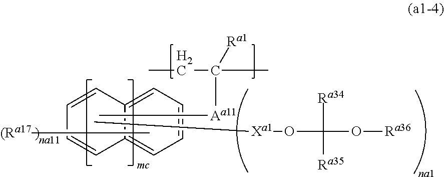
wherein
R^{a1} represents a hydrogen atom, a halogen atom, or an alkyl group having 1 to 6 carbon atoms and optionally having a halogen atom;
R^{a17} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group;
A^{a11} represents a single bond or an alkanediyl group having 1 to 12 carbon atoms, and —CH₂-contained in the alkanediyl group is optionally replaced with —O—, —CO— or —NR^{a18}—;
R^{a18} represents a hydrogen atom or an alkyl group having 1 to 6 carbon atoms;
X^{a1} represents a single bond or a carbonyl group;
R^{a34} and R^{a35} each independently represent a hydrogen atom or a hydrocarbon group having 1 to 12 carbon atoms, and R^{a36} represents a hydrocarbon group having 1 to 20 carbon atoms, or R^{a34} represents a hydrogen atom or a hydrocarbon group having 1 to 12 carbon atoms, and R^{a35} and R^{a36} are bonded to each other to form a divalent heterocyclic group having 3 to 20 carbon atoms together with —C—O— to which they are bonded, and —CH₂— contained in the hydrocarbon group and the divalent heterocyclic group is optionally replaced with —O— or —S—;
na1 represents an integer of 1 to 5, and when na1 is 2 or larger, a plurality of groups in parentheses may be the same or different from each other;
na11 represents an integer of 0 to 4, and when na11 is 2 or larger, a plurality of R^{a17} may be the same or different from each other; and
mc represents an integer of 0 to 2, 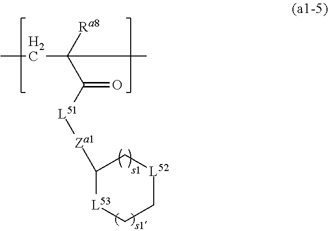
wherein
R^{a8} represents an alkyl group having 1 to 6 carbon atoms and optionally having a halogen atom, a hydrogen atom or a halogen atom;
Z^{a1} represents a single bond or *—(CH₂)ₕ₃—CO-L⁵⁴-, h3 represents an integer of 1 to 4, and * represents a bonding site to L⁵¹;
L⁵¹, L⁵², L⁵³ and L⁵⁴ each independently represent —O— or —S—;
s1 represents an integer of 1 to 3; and
s1′ represents an integer of 0 to 3, and 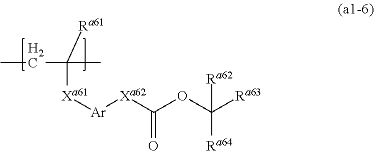
wherein
R^{a61} represents a hydrogen atom, a halogen atom, or an alkyl group having 1 to 6 carbon atoms and optionally having a halogen atom;
R^{a62}, R^{a63} and R^{a64} each independently represent an alkyl group having 1 to 6 carbon atoms or a cyclic hydrocarbon group having 3 to 18 carbon atoms and optionally having a substituent, or R^{a62} and R^{a63} are bonded to each other to form a ring having 3 to 20 carbon atoms together with a carbon atom to which they are bonded, and R⁶⁴ represents an alkyl group having 1 to 6 carbon atoms or a cyclic hydrocarbon group having 3 to 18 carbon atoms and optionally having a substituent;
X^{a61} represents a single bond, —CO—O—*, or —CO—NR^{a65}—** represents a bonding site to Ar, and R^{a65} represents a hydrogen atom or an alkyl group having 1 to 6 carbon atoms;
X^{a62} represents a single bond, *—O-L^{a61}- or *—CO—O-L^{a62}-, * represents a bonding site to Ar, and L^{a61} and L^{a62} each independently represent an alkanediyl group having 1 to 4 carbon atoms; and
Ar represents an aromatic hydrocarbon group having 6 to 20 carbon atoms and optionally having a substituent.

7. The resist composition according to claim 1, wherein the resist composition further comprises a resin that includes a structural unit represented by formula (a2-A), or
the resin that includes a structural unit represented by formula (IP) further includes a structural unit represented by formula (a2-A): 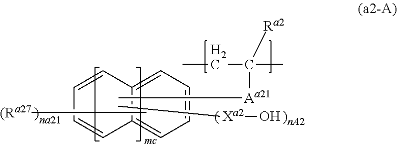
wherein
R^{a2} represents a hydrogen atom, a halogen atom, or an alkyl group having 1 to 6 carbon atoms and optionally having a halogen atom;
R^{a27} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group;
A^{a21} represents a single bond or an alkanediyl group having 1 to 12 carbon atoms, and —CH₂-contained in the alkanediyl group is optionally replaced with —O—, —CO— or —NR^{a28};
R^{a28} represents a hydrogen atom or an alkyl group having 1 to 6 carbon atoms;
X^{a2} represents a single bond or a carbonyl group;
nA2 represents an integer of 1 to 5, and when nA2 is 2 or larger, a plurality of groups in parentheses may be the same or different from each other;
na21 represents an integer of 0 to 4, and when na21 is 2 or larger, a plurality of R²⁷ may be the same or different from each other; and
mc represents an integer of 0 to 2.

8. The resist composition according to claim 1, further comprising a salt that generates an acid weaker in acidity than an acid generated from the salt represented by formula (I) or the resin that includes a structural unit represented by formula (IP).

9. A method for producing a resist pattern, comprising:
(1) a step of applying the resist composition according to claim 1 onto a substrate;
(2) a step of drying the applied resist composition to form a composition layer;
(3) a step of exposing the composition layer;
(4) a step of heating the exposed composition layer; and
(5) a step of developing the composition layer after heating.

10. A salt represented by formula (I): 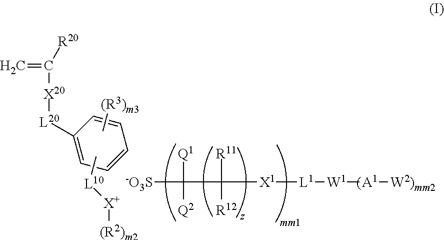
wherein
Q¹, Q², R¹¹, and R¹² each independently represent a hydrogen atom, a fluorine atom, an alkyl group having 1 to 6 carbon atoms, or a perfluoroalkyl group having 1 to 6 carbon atoms;
z represents an integer of 0 to 6, and when z is 2 or larger, a plurality of R¹¹ and R¹² may be the same or different from each other;
X¹ represents a single bond, —O—, —CO—, —S—, —SO—, —SO₂—, —NR⁷—, or a combined group of these groups;
mm1 represents 0 or 1;
L¹ represents a single bond, or a hydrocarbon group having 1 to 40 carbon atoms and optionally having a substituent, and —CH₂— contained in the hydrocarbon group is optionally replaced with —O—, —S—, —SO₂—, —SO—, —NR⁷— or —CO—;
R⁷ represents a hydrogen atom or an alkyl group having 1 to 6 carbon atoms;
W¹ represents a single bond, or a cyclic hydrocarbon group having 3 to 36 carbon atoms and optionally having a substituent, and —CH₂— contained in the cyclic hydrocarbon group is optionally replaced with —O—, —S—, —SO₂—, —SO—, —NR⁷— or —CO—;
A¹ represents a single bond, or a hydrocarbon group having 1 to 20 carbon atoms, the hydrocarbon group optionally has a substituent, and —CH₂— contained in the hydrocarbon group is optionally replaced with —O—, —S—, —SO₂—, —SO—, —NR⁷— or —CO—;
W² represents a cyclic hydrocarbon group having 3 to 36 carbon atoms and an iodine atom, —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom;
mm2 represents an integer of 1 to 5, and when mm2 is 2 or larger, a plurality of groups in parentheses may be the same or different from each other;
R² represents a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
m2 represents 1 or 2, and when m2 is 1, X is an iodine atom, and when m2 is 2, X is a sulfur atom and two R² optionally together form a ring containing a sulfur atom, where —CH₂— contained in the ring is optionally replaced with —O—, —S—, —SO₂—, —SO—, —NR⁷— or —CO—;
R³ represents a halogen atom, or a hydrocarbon group having 1 to 18 carbon atoms, the hydrocarbon group optionally has a substituent, and —CH₂— contained in the hydrocarbon group is optionally replaced with —O—, —S—, —SO₂—, —SO—, —NR⁷— or —CO—;
m3 represents an integer of 0 to 4, and when m3 is 2 or larger, a plurality of R³ may be the same or different from each other;
L¹⁰ and L²⁰ each independently represent a single bond, or a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
R²⁰ represents a hydrogen atom, a halogen atom, or an alkyl group having 1 to 6 carbon atoms and optionally having a halogen atom; and
X²⁰ represents a single bond, —O—, —CO—, —NR⁷—, —S—, —SO—, —SO₂—, a phenylene group optionally having a substituent, or a combined group of these groups.

11. The salt according to claim 10, wherein X²⁰ is a single bond or a group represented by any one of formulas (X²⁰-1) to (X²⁰-10): 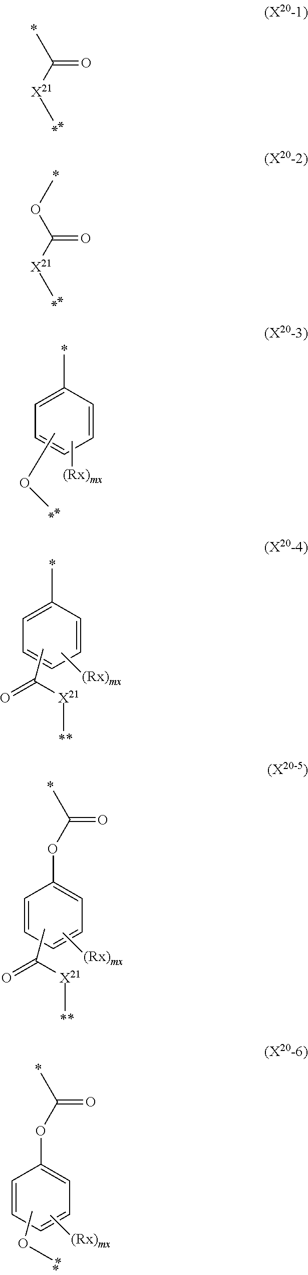 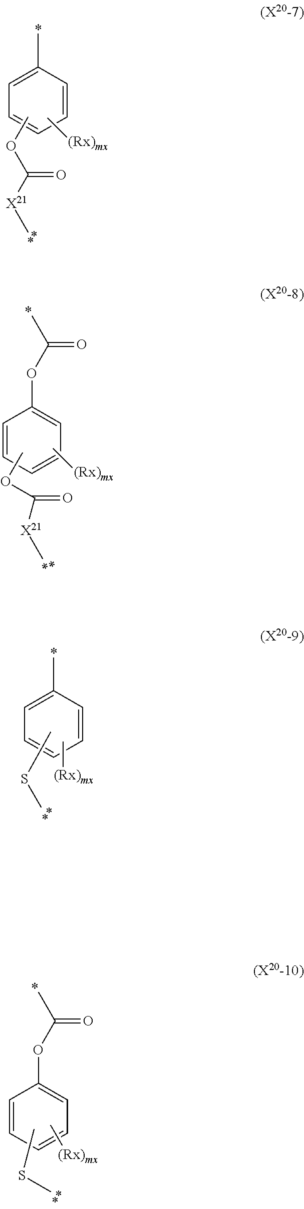
wherein
* and ** are bonding sites, and * represents a bonding site to a carbon atom to which R²⁰ is bonded;
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms;
mx represents an integer of 0 to 4; when mx is 2 or larger, a plurality of Rx may be the same or different from each other;
X²¹ represents —O— or —NR⁷—; and
R⁷ represents a hydrogen atom or an alkyl group having 1 to 6 carbon atoms.

12. The salt according to claim 10, wherein L¹ is a single bond, or a chain hydrocarbon group having 1 to 10 carbon atoms and optionally having a substituent, where —CH₂— contained in the chain hydrocarbon group is optionally replaced with —O—, —S—, —SO₂—, —SO—, —NR⁷— or —CO—; and
W¹ is a single bond, or a cyclic hydrocarbon group having 3 to 18 carbon atoms and optionally having a substituent, where —CH₂— contained in the cyclic hydrocarbon group is optionally replaced with —O—, —S—, —SO₂—, —SO—, —NR⁷— or —CO—.

13. The salt according to claim 10, wherein W² is a cyclic hydrocarbon group having 5 to 18 carbon atoms and an iodine atom, where the cyclic hydrocarbon group optionally has a substituent other than the iodine atom.

14. A resin comprising a structural unit represented by formula (IP): 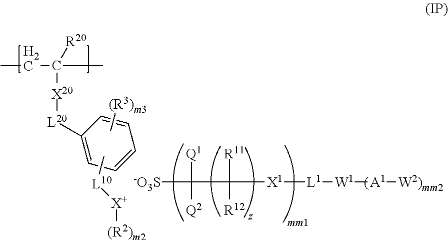
wherein
Q¹, Q², R¹¹, and R¹² each independently represent a hydrogen atom, a fluorine atom, an alkyl group having 1 to 6 carbon atoms, or a perfluoroalkyl group having 1 to 6 carbon atoms;
z represents an integer of 0 to 6, and when z is 2 or larger, a plurality of R¹¹ and R¹² may be the same or different from each other;
X¹ represents a single bond, —O—, —CO—, —S—, —SO—, —SO₂—, —NR⁷—, or a combined group of these groups;
mm1 represents 0 or 1;
L¹ represents a single bond, or a hydrocarbon group having 1 to 40 carbon atoms and optionally having a substituent, and —CH₂— contained in the hydrocarbon group is optionally replaced with —O—, —S—, —SO₂—, —SO—, —NR⁷— or —CO—;
R⁷ represents a hydrogen atom or an alkyl group having 1 to 6 carbon atoms;
W¹ represents a single bond, or a cyclic hydrocarbon group having 3 to 36 carbon atoms and optionally having a substituent, and —CH₂— contained in the cyclic hydrocarbon group is optionally replaced with —O—, —S—, —SO₂—, —SO—, —NR⁷— or —CO—;
A¹ represents a single bond, or a hydrocarbon group having 1 to 20 carbon atoms, the hydrocarbon group optionally has a substituent, and —CH₂— contained in the hydrocarbon group is optionally replaced with —O—, —S—, —SO₂—, —SO—, —NR⁷— or —CO—;
W² represents a cyclic hydrocarbon group having 3 to 36 carbon atoms and an iodine atom, —CH₂— contained in the cyclic hydrocarbon group is optionally replaced with —O—, —S—, —SO₂—, —SO—, —NR⁷— or —CO—, and the cyclic hydrocarbon group optionally has a substituent other than the iodine atom;
mm2 represents an integer of 1 to 5, and when mm2 is 2 or larger, a plurality of groups in parentheses may be the same or different from each other;
R² represents a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
m2 represents 1 or 2, and when m2 is 1, X is an iodine atom, and when m2 is 2, X is a sulfur atom and two R² optionally together form a ring containing a sulfur atom, where —CH₂— contained in the ring is optionally replaced with —O—, —S—, —SO₂—, —SO—, —NR⁷— or —CO—;
R³ represents a halogen atom, or a hydrocarbon group having 1 to 18 carbon atoms, the hydrocarbon group optionally has a substituent, and —CH₂— contained in the hydrocarbon group is optionally replaced with —O—, —S—, —SO₂—, —SO—, —NR⁷— or —CO—;
m3 represents an integer of 0 to 4, and when m3 is 2 or larger, a plurality of R³ may be the same or different from each other;
L¹⁰ and L²⁰ each independently represent a single bond, or a hydrocarbon group having 1 to 36 carbon atoms and optionally having a substituent, and —CH₂— contained in the hydrocarbon group is optionally replaced with —O—, —S—, —SO₂—, —SO—, —NR⁷— or —CO—;
R²⁰ represents a hydrogen atom, a halogen atom, or an alkyl group having 1 to 6 carbon atoms and optionally having a halogen atom; and
X²⁰ represents a single bond, —O—, —CO—, —NR⁷—, —S—, —SO—, —SO₂—, a phenylene group optionally having a substituent, or a combined group of these groups.
